(19) United States
(12) Patent Application Publication
(10) Pub. No.: US 2023/0212135 A1
(43) Pub. Date: Jul. 6, 2023

(54) LIGHT EMITTING ELEMENT, AMINE COMPOUND FOR THE SAME, AND DISPLAY DEVICE INCLUDING THE SAME

(71) Applicant: Samsung Display Co., Ltd., Yongin-si (KR)

(72) Inventor: Takao SAKUMA, Yokohama (JP)

(21) Appl. No.: 18/066,185

(22) Filed: Dec. 14, 2022

(30) Foreign Application Priority Data
Jan. 4, 2022 (KR) 10-2022-0001177

(51) Int. Cl. C07D 307/91; H10K 85/60; H10K 50/15; H10K 50/11

(52) U.S. Cl. CPC C07D 307/91; H01L 51/0072; H01L 51/0061; H01L 51/0052; H01L 51/5056; H01L 51/5012

(57) ABSTRACT

A light emitting element that includes a first electrode, a second electrode on the first electrode, an emission layer between the first electrode and the second electrode, and a hole transport region which is between the first electrode and the emission layer is provided. The emission layer includes an amine compound represented by Formula 1:

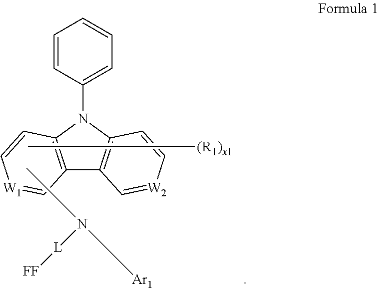

### CROSS-REFERENCE TO RELATED APPLICATION

This application claims priority to and the benefit of Korean Patent Application No. 10-2022-0001177, filed on Jan. 4, 2022, in the Korean Intellectual Property Office, the entire content of which is hereby incorporated by reference.

### BACKGROUND

### 1. Field

Aspects of one or more embodiments of the present disclosure herein relate to an amine compound, a light emitting element including the same, and for example, to a light emitting element including a novel amine compound in a hole transport region.

### 2. Description of Related Art

Recently, the development of an organic electroluminescence display device as an image display device is being actively conducted. The organic electroluminescence display device includes a self-luminescent light emitting element in which holes and electrons injected from a first electrode and a second electrode recombine in an emission layer, and thus a luminescent material of the emission layer emits light to implement display.

In the application of a light emitting element to a display device, there is a demand for a light emitting element having low driving voltage, high luminous efficiency, and a long service life, and development on materials for a light emitting element capable of stably attaining such characteristics is being continuously required (sought).

Development on materials for a hole transport region having improved electronic stability is being carried out in order to implement a light emitting element having low driving voltage.

### SUMMARY

An aspect of one or more embodiments of the present disclosure is directed toward an amine compound having an improvement in the stability of material.

Another aspect of one or more embodiments of the present disclosure is directed toward a light emitting element exhibiting low driving voltage and a display device including the same.

Additional aspects will be set forth in part in the description which follows and, in part, will be apparent from the description, or may be learned by practice of the presented embodiments of the disclosure.

An embodiment of the present disclosure provides a light emitting element including: a first electrode; a second electrode on the first electrode; an emission layer between the first electrode and the second electrode; and a hole transport region which is between the first electrode and the emission layer, and includes an amine compound represented by Formula 1:

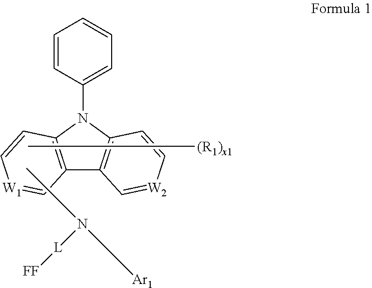

In Formula 1, Ar₁ may be a substituted or unsubstituted aryl group having 6 to 50 ring-forming carbon atoms, or a substituted or unsubstituted heteroaryl group having 2 to 50 ring-forming carbon atoms, and the embodiment in which Ar₁ is a substituted or unsubstituted carbazole group is excluded, L is a substituted or unsubstituted arylene group having 6 to 50 ring-forming carbon atoms, or a substituted or unsubstituted heteroarylene group having 2 to 50 ring-forming carbon atoms, R₁ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, x1 is an integer from 0 to 5, W₁ and W₂ are each independently CRₐ, or a carbon atom bonded to the nitrogen atom in Formula 1, Rₐ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, and FF is represented by Formula 2, and in Formula 2, “—*” is a position linked to L:

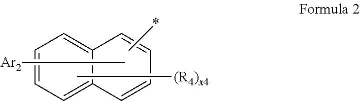

In Formula 2, Ar₂ may be a substituted or unsubstituted aryl group having 6 to 50 ring-forming carbon atoms, and the embodiment in which Ar₂ is an aryl group substituted with an amine group is excluded, R₄ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, x4 is an integer from 0 to 6, and in the embodiment when R₄ does not form a ring with an adjacent group, in Formula 1, each of W₁ and W₂ is CRₐ, and when Formula 2 is represented by

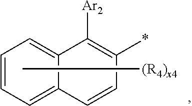

R₄ does not form a ring with an adjacent group, and Ar₁ is a substituted or unsubstituted fluorene group, and the embodiment in which the nitrogen atom in Formula 1 is linked to the second position of the substituted or unsubstituted fluorene group, Ar₁, is excluded.

In an embodiment, FF may be represented by Formula 2a:

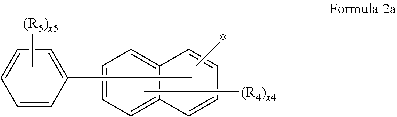

In Formula 2a, R₅ may be a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, x5 is an integer from 0 to 5, and R₄ and x4 are the same as defined in Formula 2.

In an embodiment, FF may be represented by any one selected from among Formula 2-1 to Formula 2-5:

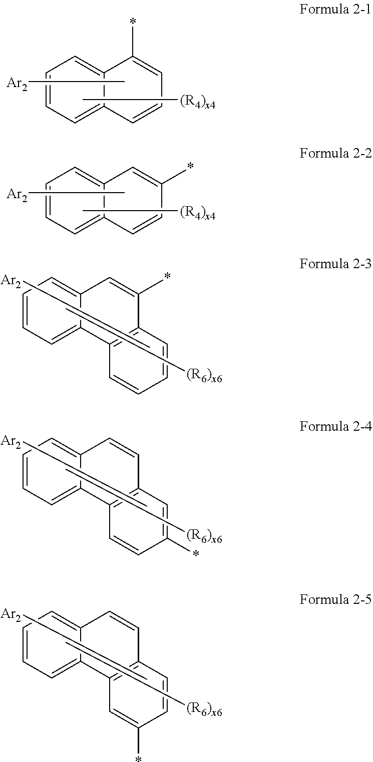

In Formula 2-3 to Formula 2-5, R₆ may be a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, and x6 is an integer from 0 to 8, and in Formula 2-1 to Formula 2-5, Ar₂, R₄, and x4 are the same as defined in Formula 2.

In an embodiment, FF may be represented by any one selected from among Formula 2-3a, Formula 2-4a, and Formula 2-5a:

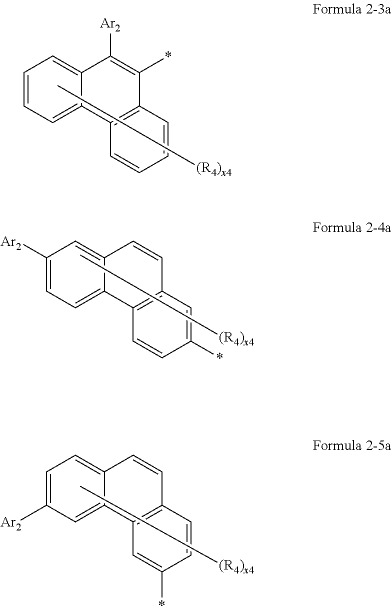

In Formula 2-3a, Formula 2-4a, and Formula 2-5a, Ar₂, R₄, and x4 are the same as defined in Formula 2.

In an embodiment, the amine compound represented by Formula 1 may be represented by any one selected from among Formula 3-1 to Formula 3-3:

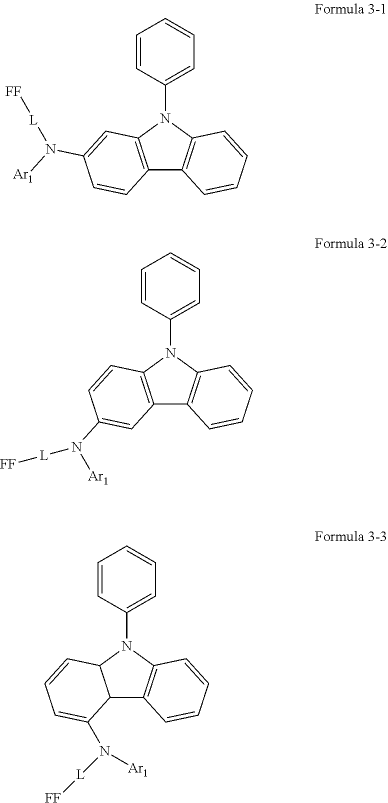

In Formula 3-1 to Formula 3-3, L, Ar₁, and FF are the same as defined in Formula 1.

In an embodiment, L may be a substituted or unsubstituted phenylene group, a substituted or unsubstituted biphenylene group, or a substituted or unsubstituted divalent dibenzofuran group.

In an embodiment, Ar₁ may be a substituted or unsubstituted phenyl group, a substituted or unsubstituted biphenyl group, a substituted or unsubstituted terphenyl group, a substituted or unsubstituted quaterphenyl group, a substituted or unsubstituted fluorenyl group, a substituted or unsubstituted naphthyl group, a substituted or unsubstituted phenanthryl group, a substituted or unsubstituted dibenzofuran group, or a substituted or unsubstituted dibenzothiophene group.

In an embodiment, the hole transport region may further include a compound represented by Formula H-1:

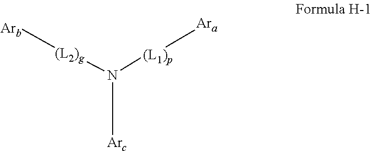

In Formula H-1, Arₐ and Ar_{b} may each independently be a substituted or unsubstituted aryl group having 6 to 30 ring-forming carbon atoms, or a substituted or unsubstituted heteroaryl group having 2 to 30 ring-forming carbon atoms, Ar_{c} is a substituted or unsubstituted aryl group having 6 to 30 ring-forming carbon atoms, L₁ and L₂ are each independently a direct linkage, a substituted or unsubstituted arylene group having 6 to 30 ring-forming carbon atoms, or a substituted or unsubstituted heteroarylene group having 2 to 30 ring-forming carbon atoms, and p and q are each independently an integer from 0 to 10.

In an embodiment, the emission layer may include a compound represented by Formula E-1:

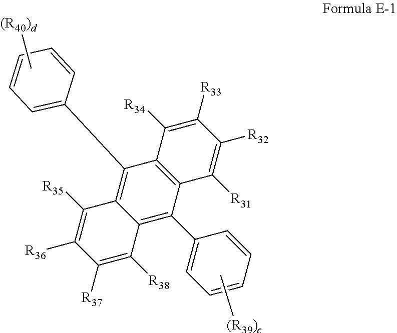

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring, and c and d are each independently an integer from 0 to 5.

In an embodiment, the hole transport region may include a hole injection layer on the first electrode, a hole transport layer on the hole injection layer, and an electron blocking layer on the hole transport layer, and the hole transport layer may include the amine compound represented by Formula 1.

In an embodiment, the amine compound may be a monoamine compound.

In an embodiment of the present disclosure, an amine compound may be represented by Formula 1:

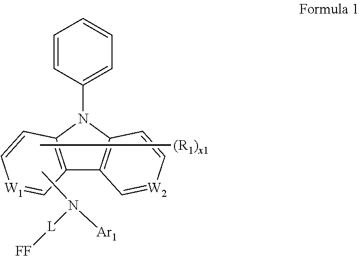

In Formula 1, Ar₁ may be a substituted or unsubstituted aryl group having 6 to 50 ring-forming carbon atoms, or a substituted or unsubstituted heteroaryl group having 2 to 50 ring-forming carbon atoms, and the embodiment in which Ar₁ is a substituted or unsubstituted carbazole group is excluded, L is a substituted or unsubstituted arylene group having 6 to 50 ring-forming carbon atoms, or a substituted or unsubstituted heteroarylene group having 2 to 50 ring-forming carbon atoms, R₁ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, x1 is an integer from 0 to 5, W₁ and W₂ are each independently CRₐ, or a carbon atom bonded to the nitrogen atom in Formula 1, Rₐ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, and FF is represented by Formula 2, and in Formula 2, “—*” is a position linked to L:

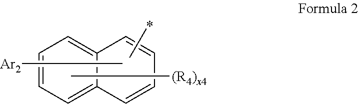

In Formula 2, Ar₂ may be a substituted or unsubstituted aryl group having 6 to 50 ring-forming carbon atoms, and the embodiment in which Ar₂ is an aryl group substituted with an amine group is excluded, R₄ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, x4 is an integer from 0 to 6, and in the embodiment when R₄ does not form a ring with an adjacent group, in Formula 1, each of W₁ and W₂ is CRₐ, and where, when Formula 2 is represented by

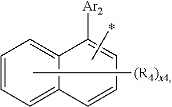

R₄ does not form a ring with an adjacent group, and Ar₁ is a substituted or unsubstituted fluorene group, and the embodiment in which the nitrogen atom in Formula 1 is linked to the second position of the substituted or unsubstituted fluorene group, Ar₁, is excluded.

In an embodiment of the present disclosure, a display device includes a plurality of light emitting elements, and each of the light emitting elements includes: a first electrode; a second electrode on the first electrode; an emission layer between the first electrode and the second electrode; and a hole transport region which is between the first electrode and the emission layer, and includes an amine compound represented by Formula 1:

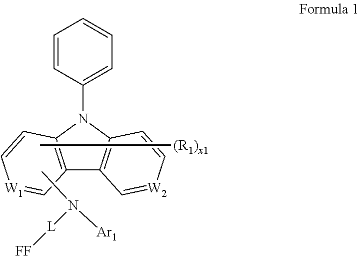

In Formula 1, Ar₁ may be a substituted or unsubstituted aryl group having 6 to 50 ring-forming carbon atoms, or a substituted or unsubstituted heteroaryl group having 2 to 50 ring-forming carbon atoms, and the embodiment in which Ar₁ is a substituted or unsubstituted carbazole group is excluded, L is a substituted or unsubstituted arylene group having 6 to 50 ring-forming carbon atoms, or a substituted or unsubstituted heteroarylene group having 2 to 50 ring-forming carbon atoms, R₁ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, x1 is an integer from 0 to 5, W₁ and W₂ are each independently CRₐ, or a carbon atom bonded to the nitrogen atom in Formula 1, Rₐ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, and FF is represented by Formula 2, and in Formula 2, “—*” is a position linked to L:

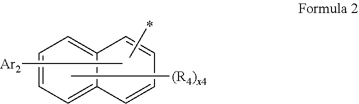

In Formula 2, Ar₂ may be a substituted or unsubstituted aryl group having 6 to 50 ring-forming carbon atoms, and the embodiment in which Ar₂ is an aryl group substituted with an amine group is excluded, R₄ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, x4 is an integer from 0 to 6, and in the embodiment when R₄ does not form a ring with an adjacent group, in Formula 1, each of W₁ and W₂ is CRₐ, and when Formula 2 is represented by

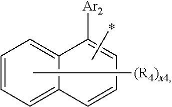

R₄ does not form a ring with an adjacent group, and Ar₁ is a substituted or unsubstituted fluorene group, and the embodiment in which the nitrogen atom in Formula 1 is linked to the second position of the substituted or unsubstituted fluorene group, Ar₁, is excluded.

In an embodiment, the plurality of light emitting elements may include a first light emitting element including a first emission layer that emits light having a first wavelength; a second light emitting element including a second emission layer that emits light having a second wavelength different from the first wavelength and is spaced apart from (separated from) the first emission layer on a plane; and a third light emitting element that emits light having a third wavelength different from the first wavelength and the second wavelength and is spaced apart from (separated from) the first emission layer and the second emission layer on a plane.

In an embodiment, the first wavelength may be longer than the second wavelength, and the second wavelength may be longer than the third wavelength.

### BRIEF DESCRIPTION OF THE DRAWINGS

The accompanying drawings are included to provide a further understanding of the present disclosure, and are incorporated in and constitute a part of this specification. The drawings illustrate example embodiments of the present disclosure and, together with the description, serve to explain principles of the present disclosure. In the drawings:

FIG. 1 is a plan view illustrating a display device according to an embodiment;

FIG. 2 is a cross-sectional view of a display device according to an embodiment;

FIG. 3 is a cross-sectional view schematically illustrating a light emitting element according to an embodiment;

FIG. 4 is a cross-sectional view schematically illustrating a light emitting element according to an embodiment;

FIG. 5 is a cross-sectional view schematically illustrating a light emitting element according to an embodiment;

FIG. 6 is a cross-sectional view schematically illustrating a light emitting element according to an embodiment;

FIG. 7 is a cross-sectional view of a display device according to an embodiment;

FIG. 8 is a cross-sectional view of a display device according to an embodiment;

FIG. 9 is a cross-sectional view illustrating a display device according to an embodiment; and

FIG. 10 is a cross-sectional view illustrating a display device according to an embodiment.

### DETAILED DESCRIPTION

The present disclosure may be modified in many alternate forms, and thus specific embodiments will be exemplified in the drawings and described in this text in more detail. It should be understood, however, that it is not intended to limit the present disclosure to the particular forms disclosed, but rather, is intended to cover all modifications, equivalents, and alternatives falling within the spirit and scope of the present disclosure.

When explaining each of drawings, like reference numerals are used for referring to like elements. In the accompanying drawings, the dimensions of each structure may be exaggeratingly illustrated for clarity of the present disclosure. It will be understood that, although the terms “first”, “second”, etc. may be used herein to describe one or more suitable components, these components should not be limited by these terms. These terms are only used to distinguish one component from another. For example, a first component could be termed a second component, and, similarly, a second component could be termed a first component, without departing from the scope of the present disclosure. As used herein, the singular forms, “a,” “an,” and “the” are intended to include the plural forms as well, unless the context clearly indicates otherwise.

In the present disclosure, it will be understood that the meaning of “comprise” or “have” specifies the presence of a feature, a fixed number, a step, a process, an element, a component, or a combination thereof disclosed in the disclosure, but does not exclude the possibility of presence or addition of one or more other features, fixed numbers, steps, processes, elements, components, or combination thereof.

In the present disclosure, when a layer, a film, a region, or a plate is referred to as being “above” or “in an upper portion” another layer, film, region, or plate, it can be not only directly on the layer, film, region, or plate, but intervening layers, films, regions, or plates may also be present. In contrast, when a part such as a layer, a film, a region, or a plate is referred to as being “under” or “below” another part, it can be directly under the other part, or an intervening part may also be present. In some embodiments, in the disclosure, it will be understood that when a part is referred to as being disposed “on” another part, it may be disposed on an upper portion of the another part, or disposed on a lower portion of the another part as well.

In the disclosure, the term “substituted or unsubstituted” may refer to substituted or unsubstituted with at least one substituent selected from the group including (e.g., consisting of) a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In some embodiments, each of the substituents exemplified above may be substituted or unsubstituted. For example, a biphenyl group may be interpreted as an aryl group or a phenyl group substituted with a phenyl group.

In the disclosure, the phrase “bonded to an adjacent group to form a ring” may indicate that one is bonded to an adjacent group to form a substituted or unsubstituted hydrocarbon ring, or a substituted or unsubstituted heterocycle. The hydrocarbon ring includes an aliphatic hydrocarbon ring and/or an aromatic hydrocarbon ring. The heterocycle includes an aliphatic heterocycle and/or an aromatic heterocycle. The hydrocarbon ring and the heterocycle may be monocyclic or polycyclic. In some embodiments, the rings formed by being bonded to each other may be connected to another ring to form a spiro structure.

In the disclosure, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In some embodiments, two methyl groups in 4,5-dimethylphenanthrene may be interpreted as “adjacent groups” to each other.

In the disclosure, examples of the halogen atom may include a fluorine atom, a chlorine atom, a bromine atom, or an iodine atom.

In the disclosure, the alkyl group may be a linear, branched or cyclic type or kind. The number of carbons in the alkyl group may be 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but the embodiment of the present disclosure is not limited thereto.

In the disclosure, an alkenyl group refers to a hydrocarbon group including at least one carbon double bond in the middle or terminal of an alkyl group having at least two carbon atoms. The alkenyl group may be a linear chain or a branched chain. The carbon number is not limited, but may be 2 to 30, 2 to 20 or 2 to 10. Examples of the alkenyl group include a vinyl group, a 1-butenyl group, a 1-pentenyl group, a 1,3-butadienyl aryl group, a styrenyl group, a styrylvinyl group, etc., without limitation.

The hydrocarbon ring group herein refers to any functional group or substituent derived from an aliphatic hydrocarbon ring. The hydrocarbon ring group may be a saturated hydrocarbon ring group having 5 to 20 ring-forming carbon atoms.

In the disclosure, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but the embodiment of the present disclosure is not limited thereto.

In the disclosure, the fluorenyl group may be substituted, and two substituents may be bonded to each other to form a spiro structure. Examples of embodiments in which the fluorenyl group is substituted are as follows. However, the embodiment of the present disclosure is not limited thereto.

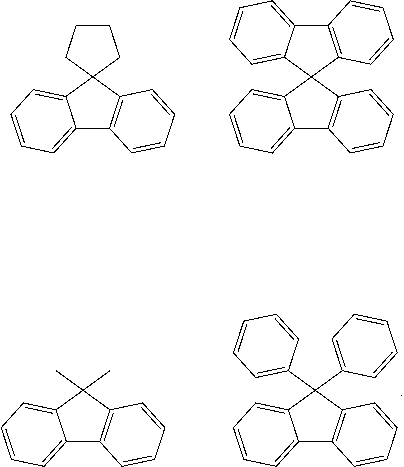

The heterocyclic group herein refers to any functional group or substituent derived from a ring including at least one of B, O, N, P, Si, or Se as a heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

In the disclosure, the heterocyclic group may include at least one of B, O, N, P, Si or S as a heteroatom. When the heterocyclic group includes two or more heteroatoms, the two or more heteroatoms may be the same or different. The heterocyclic group may be a monocyclic heterocyclic group or a polycyclic heterocyclic group and includes a heteroaryl group. The number of ring-forming carbon atoms in the heterocyclic group may be 2 to 30, 2 to 20, or 2 to 10.

In the disclosure, the aliphatic heterocyclic group may include one or more selected from among B, O, N, P, Si, and S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but the embodiment of the present disclosure is not limited thereto.

The heteroaryl group herein may include at least one of B, O, N, P, Si, or S as a heteroatom. When the heteroaryl group contains two or more heteroatoms, the two or more heteroatoms may be the same as or different from each other. The heteroaryl group may be a monocyclic heteroaryl group or polycyclic heteroaryl group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a triazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but the embodiment of the present disclosure is not limited thereto.

In the disclosure, the above description of the aryl group may be applied to an arylene group except that the arylene group is a divalent group. The above description of the heteroaryl group may be applied to a heteroarylene group except that the heteroarylene group is a divalent group.

In the disclosure, the silyl group includes an alkylsilyl group and/or an arylsilyl group. Examples of the silyl group may include trimethylsilyl, triethylsilyl, t-butyldimethylsilyl, vinyldimethylsilyl, propyldimethylsilyl, triphenylsilyl, diphenylsilyl, phenylsilyl, etc. However, an embodiment of the present disclosure is not limited thereto.

In the disclosure, a thio group may include an alkylthio group and/or an arylthio group. The thio group may refer to a sulfur atom that is bonded to the alkyl group or the aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but the embodiment of the present disclosure is not limited thereto.

In the disclosure, an oxy group may refer to an oxygen atom that is bonded to the alkyl group or the aryl group as defined above. The oxy group may include an alkoxy group and/or an aryl oxy group. The alkoxy group may be a linear chain, a branched chain or a ring chain. The number of carbon atoms in the alkoxy group is not limited, but may be, for example, 1 to 20 or 1 to 10. Examples of the oxy group include methoxy, ethoxy, n-propoxy, isopropoxy, butoxy, pentyloxy, hexyloxy, octyloxy, nonyloxy, decyloxy, benzyloxy, etc., but the embodiment of the present disclosure is not limited thereto.

In the disclosure, the alkenyl group may be linear or branched. The number of carbon atoms in the alkynyl group is not limited, but may be 2 to 30, 2 to 20, or 2 to 10. Examples of the alkenyl group include a vinyl group, a 1-butenyl group, a 1-pentenyl group, a 1,3-butadienyl aryl group, a styrenyl group, a styrylvinyl group, etc., without limitation.

In the disclosure, the number of carbon atoms in an amine group is not limited, but may be 1 to 30. The amine group may include an alkyl amine group and/or an aryl amine group. Examples of the amine group may include a methylamine group, a dimethylamine group, a phenylamine group, a diphenylamine group, a naphthylamine group, a 9-methyl-anthracenylamine group, a triphenylamine group, etc., but the embodiment of the present disclosure is not limited thereto.

In the disclosure, the alkyl group may be selected from among an alkylthio group, an alkylsulfoxy group, an alkylaryl group, an alkylamino group, an alkyl boron group, an alkyl silyl group, and an alkyl amine group is the same as the examples of the alkyl group described above.

In the disclosure, the aryl group may be selected from among an aryloxy group, an arylthio group, an arylsulfoxy group, an arylamino group, an arylboron group, an arylsilyl group, an arylamine group is the same as the examples of the aryl group described above.

In the disclosure, a direct linkage may refer to a single bond.

In some embodiments, in the disclosure, “ [symbol] ” and “—*” refer to a position to be connected.

Hereinafter, embodiments of the present disclosure will be described with reference to the accompanying drawings.

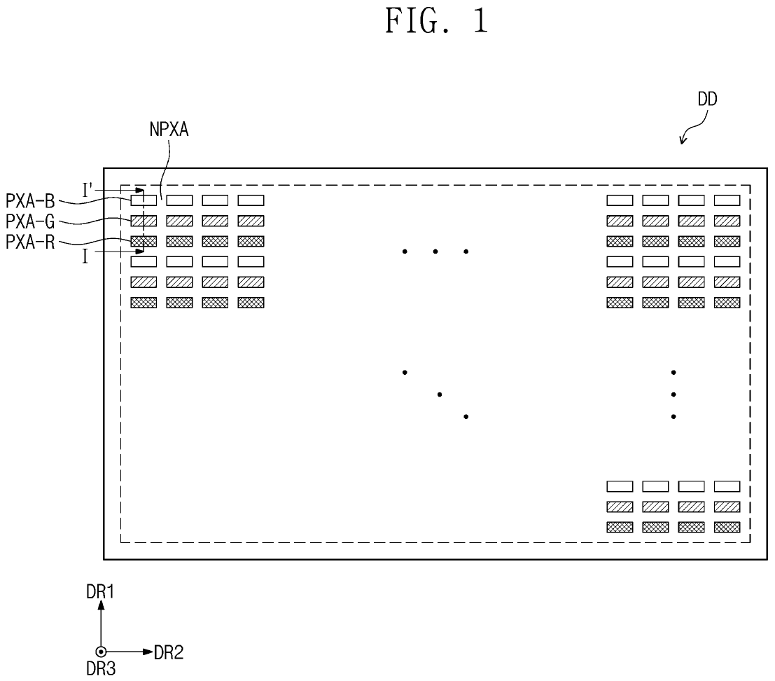

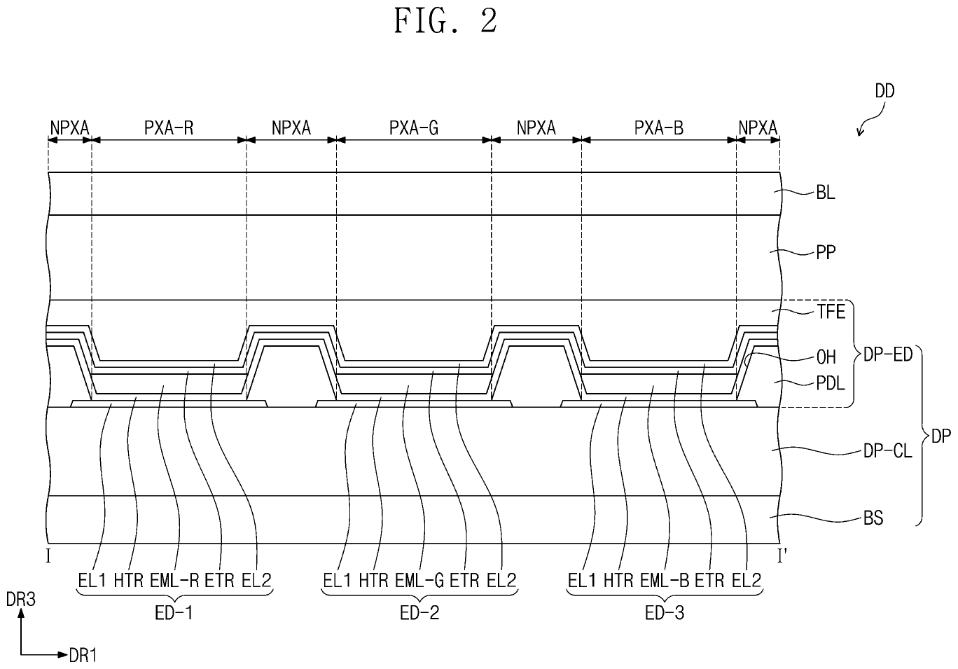

FIG. 1 is a plan view illustrating an embodiment of a display device DD. FIG. 2 is a cross-sectional view of the display device DD of the embodiment. FIG. 2 is a cross-sectional view illustrating a part taken along line I-I′ of FIG. 1.

The display device DD may include a display panel DP and an optical layer PP on the display panel DP. The display panel DP includes light emitting elements ED-1, ED-2, and ED-3. The display device DD may include a plurality of light emitting elements ED-1, ED-2, and ED-3.

The optical layer PP may be on the display panel DP to control reflected light in the display panel DP due to external light. The optical layer PP may include, for example, a polarization layer or a color filter layer. In some embodiments, the optical layer PP may not be provided in the display device DD of an embodiment.

A base substrate BL may be on the optical layer PP. The base substrate BL may be a member which provides a base surface on which the optical layer PP disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In some embodiments, the base substrate BL may not be provided.

The display device DD according to an embodiment may further include a filling layer. The filling layer may be between a display element layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display element layer DP-ED. The display element layer DP-ED may include a pixel defining film PDL, the light emitting elements ED-1, ED-2, and ED-3 disposed between portions of the pixel defining film PDL, and an encapsulation layer TFE on the light emitting elements ED-1, ED-2, and ED-3.

The base layer BS may be a member which provides a base surface on which the display element layer DP-ED is disposed. The base layer BS may be a glass substrate, a metal substrate, a plastic substrate, etc. However, the embodiment is not limited thereto, and the base layer BS may be an inorganic layer, an organic layer, or a composite material layer.

In an embodiment, the circuit layer DP-CL is on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light emitting elements ED-1, ED-2, and ED-3 of the display element layer DP-ED.

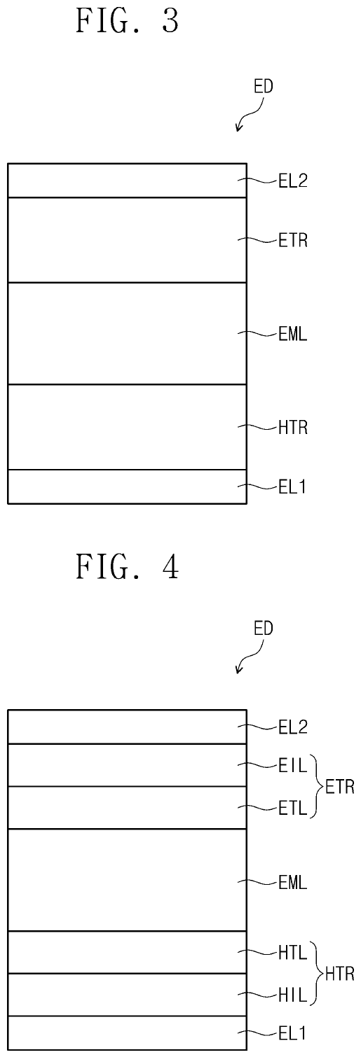

Each of the light emitting elements ED-1, ED-2, and ED-3 may have a structure of a light emitting element ED of an embodiment according to FIGS. 3 to 6, which will be described in more detail. Each of the light emitting elements ED-1, ED-2 and ED-3 may include a first electrode EL1, a hole transport region HTR, emission layers EML-R, EML-G and EML-B, an electron transport region ETR, and a second electrode EL2.

FIG. 2 illustrates an embodiment in which the emission layers EML-R, EML-G, and EML-B of the light emitting elements ED-1, ED-2, and ED-3 are disposed in openings OH defined in the pixel defining film PDL, and the hole transport region HTR, the electron transport region ETR, and the second electrode EL2 are provided as a common layer in the entire light emitting elements ED-1, ED-2, and ED-3. However, the embodiment of the present disclosure is not limited thereto. For example, the hole transport region HTR and the electron transport region ETR in an embodiment may be provided by being patterned inside the openings OH defined in the pixel defining film PDL. For example, the hole transport region HTR, the emission layers EML-R, EML-G, and EML-B, and the electron transport region ETR of the light emitting elements ED-1, ED-2, and ED-3 in an embodiment may be provided by being patterned in an inkjet printing method.

The encapsulation layer TFE may cover the light emitting elements ED-1, ED-2 and ED-3. The encapsulation layer TFE may seal the display element layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE according to an embodiment may include at least one inorganic film (hereinafter, an encapsulation-inorganic film). The encapsulation layer TFE according to an embodiment may also include at least one organic film (hereinafter, an encapsulation-organic film) and at least one encapsulation-inorganic film.

The encapsulation-inorganic film protects (reduces the moisture/oxygen) the display element layer DP-ED from moisture/oxygen, and the encapsulation-organic film protects (reduces foreign substances) the display element layer DP-ED from foreign substances such as dust particles. The encapsulation-inorganic film may include silicon nitride, silicon oxynitride, silicon oxide, titanium oxide, aluminum oxide, and/or the like, but the embodiment of the present disclosure is not limited thereto. The encapsulation-organic film may include an acrylic-based compound, an epoxy-based compound, or the like. The encapsulation-organic film may include a photopolymerizable organic material, but the embodiment of the present disclosure is not limited thereto.

The encapsulation layer TFE may be on the second electrode EL2 and may be disposed filling the opening OH.

Referring to FIGS. 1 and 2, the display device DD may include a non-light emitting region NPXA and light emitting regions PXA-R, PXA-G and PXA-B. The light emitting regions PXA-R, PXA-G and PXA-B may be regions in which light generated by the respective light emitting elements ED-1, ED-2 and ED-3 is emitted. The light emitting regions PXA-R, PXA-G, and PXA-B may be spaced apart from each other on a plane (e.g., in a plan view).

Each of the light emitting regions PXA-R, PXA-G, and PXA-B may be a region divided by the pixel defining film PDL. The non-light emitting regions NPXA may be regions between the adjacent light emitting regions PXA-R, PXA-G, and PXA-B, which correspond to portions of the pixel defining film PDL. In some embodiments, in the disclosure, the light emitting regions PXA-R, PXA-G, and PXA-B may respectively correspond to pixels. The pixel defining film PDL may divide the light emitting elements ED-1, ED-2, and ED-3. The emission layers EML-R, EML-G and EML-B of the light emitting elements ED-1, ED-2 and ED-3 may be disposed in openings OH defined in the pixel defining film PDL and separated from each other.

The light emitting regions PXA-R, PXA-G and PXA-B may be divided into a plurality of groups according to the color of light generated from the light emitting elements ED-1, ED-2 and ED-3. In the display device DD of an embodiment shown in FIGS. 1 and 2, three light emitting regions PXA-R, PXA-G, and PXA-B which emit red light, green light, and blue light, respectively are illustrated. For example, the display device DD of an embodiment may include the red light emitting region PXA-R, the green light emitting region PXA-G, and the blue light emitting region PXA-B that are separated from each other.

In the display device DD according to an embodiment, the plurality of light emitting elements ED-1, ED-2 and ED-3 may emit light beams having wavelengths different from each other. For example, in an embodiment, the display device DD may include a first light emitting element ED-1 that emits red light, a second light emitting element ED-2 that emits green light, and a third light emitting element ED-3 that emits blue light. For example, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, the embodiment of the present disclosure is not limited thereto, and the first to third light emitting elements ED-1, ED-2, and ED-3 may emit light beams in substantially the same wavelength range or at least one light emitting element may emit a light beam in a wavelength range different from the others. For example, the first to third light emitting elements ED-1, ED-2, and ED-3 may all emit blue light.

The light emitting regions PXA-R, PXA-G, and PXA-B in the display device DD according to an embodiment may be arranged in a stripe form. Referring to FIG. 1, the plurality of red light emitting regions PXA-R may be arranged with each other along a second direction axis DR2, the plurality of green light emitting regions PXA-G may be arranged with each other along a second direction axis DR2, and the plurality of blue light emitting regions PXA-B each may be arranged with each other along the second direction DR2. In some embodiments, the red light emitting region PXA-R, the green light emitting region PXA-G, and the blue light emitting region PXA-B may be alternately arranged in this order along the first direction DR1. (DR3 is a third direction which is normal or perpendicular to the plane defined by the first direction DR1 and the second direction DR2)

FIGS. 1 and 2 illustrate that all the light emitting regions PXA-R, PXA-G, and PXA-B have a substantially similar area, but the embodiment of the present disclosure is not limited thereto. Thus, the light emitting regions PXA-R, PXA-G, and PXA-B may have different areas from each other according to the wavelength range of the emitted light. In this embodiment, the areas of the light emitting regions PXA-R, PXA-G, and PXA-B may refer to areas when viewed in a plane defined by the first direction DR1 and the second direction DR2.

In some embodiments, an arrangement form of the light emitting regions PXA-R, PXA-G, and PXA-B is not limited to the feature illustrated in FIG. 1, and the order in which the red light emitting region PXA-R, the green light emitting region PXA-G, and the blue light emitting region PXA-B are arranged may be provided in one or more suitable combinations according to the characteristics of display quality required in the display device DD. For example, the arrangement form of the light emitting regions PXA-R, PXA-G, and PXA-B may be a PENTILE® arrangement form (for example, an RGBG matrix, an RGBG structure, or RGBG matrix structure) or a diamond arrangement form (Diamond Pixel™ arrangement form) (e.g., a display (e.g., an OLED display) containing red, blue, and green (RGB) light emitting regions arranged in the shape of diamonds). PENTILE® is a duly registered trademark of Samsung Display Co., Ltd. Diamond Pixel™ is a trademark of Samsung Display Co., Ltd.

In some embodiments, the areas of the light emitting regions PXA-R, PXA-G, and PXA-B may be different from each other. For example, in an embodiment, the area of the green light emitting region PXA-G may be smaller than that of the blue light emitting region PXA-B, but the embodiment of the present disclosure is not limited thereto.

Hereinafter, FIGS. 3 to 6 are cross-sectional views schematically illustrating light emitting elements according to embodiments. The light emitting elements ED according to embodiments each may include a first electrode EL1, a second electrode EL2 facing the first electrode EL1, and at least one functional layer between the first electrode EL1 and the second electrode EL2. Each of the light emitting elements ED of embodiments may include an amine compound of an embodiment, which will be described in more detail, in at least one functional layer.

Each of the light emitting elements ED may include, as at least one functional layer, a hole transport region HTR, an emission layer EML, and an electron transport region ETR that are sequentially stacked. Referring to FIG. 3, the light emitting element ED of an embodiment may include a first electrode EL1, a hole transport region HTR, an emission layer EML, an electron transport region ETR, and a second electrode EL2 that are sequentially stacked.

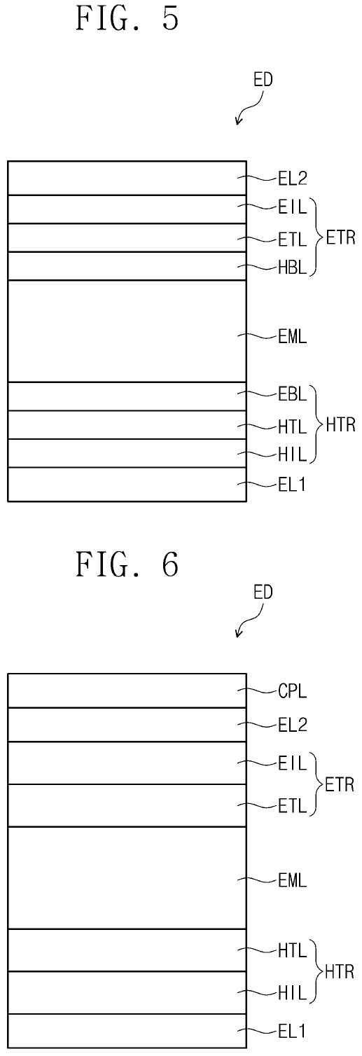

Compared with FIG. 3, FIG. 4 illustrates a cross-sectional view of a light emitting element ED of an embodiment, in which a hole transport region HTR includes a hole injection layer HIL and a hole transport layer HTL, and an electron transport region ETR includes an electron injection layer EIL and an electron transport layer ETL. In addition, compared with FIG. 3, FIG. 5 illustrates a cross-sectional view of a light emitting element ED of an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light emitting element ED of an embodiment including a capping layer CPL on a second electrode EL2.

The light emitting element ED of an embodiment may include an amine compound of an embodiment, which will be described in more detail, in the hole transport region HTR. In the light emitting element ED of an embodiment, at least one of a hole injection layer HIL, a hole transport layer HTL, or an electron blocking layer EBL in the hole transport region HTR may include the amine compound of an embodiment.

When the first electrode EL1 is the transmissive electrode, the first electrode EL1 may include a transparent metal oxide such as indium tin oxide (ITO), indium zinc oxide (IZO), zinc oxide (ZnO), or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, one or more compounds or mixtures thereof (e.g., a mixture of Ag and Mg). In some embodiments, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layer structure of ITO/Ag/ITO, but the embodiment of the present disclosure is not limited thereto. In addition, the embodiment of the present disclosure is not limited thereto, and the first electrode EL1 may include the above-described metal materials, combinations of at least two metal materials of the above-described metal materials, oxides of the above-described metal materials, and/or the like. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may have a single layer formed of a single material, a single layer formed of a plurality of different materials, or a multilayer structure including a plurality of layers formed of a plurality of different materials.

The hole transport region HTR may include at least one of the hole injection layer HIL, the hole transport layer HTL, or the electron blocking layer EBL. In some embodiments, the hole transport region HTR may include a plurality of stacked hole transport layers.

In some embodiments, the hole transport region HTR may have a single layer structure of the hole injection layer HIL or the hole transport layer HTL, or may have a single layer structure formed of a hole injection material and a hole transport material. In an embodiment, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, or a hole transport layer HTL/buffer layer are stacked in order from the first electrode EL1, but the embodiment of the present disclosure is not limited thereto.

The thickness of the hole transport region HTR may be, for example, from about 50 Å to about 15,000 Å. The hole transport region HTR may be formed using one or more suitable methods such as a vacuum deposition method, a spin coating method, a cast method, a Langmuir-Blodgett (LB) method, an inkjet printing method, a laser printing method, and/or a laser induced thermal imaging (LITI) method.

The light emitting element ED of an embodiment may include the amine compound represented by Formula 1 of an embodiment in the hole transport region HTR. The hole transport layer HTL in the light emitting element ED of an embodiment may include the amine compound represented by Formula 1 of an embodiment:

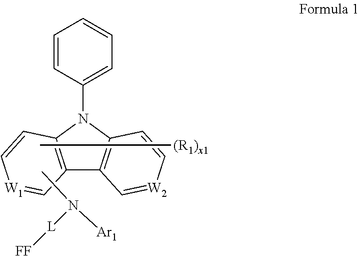

In Formula 1, Ar₁ is a substituted or unsubstituted aryl group having 6 to 50 ring-forming carbon atoms, or a substituted or unsubstituted heteroaryl group having 2 to 50 ring-forming carbon atoms, and the embodiment in which Ar₁ is a substituted or unsubstituted carbazole group is excluded. For example, Ar₁ may be a substituted or unsubstituted phenyl group, a substituted or unsubstituted biphenyl group, a substituted or unsubstituted terphenyl group, a substituted or unsubstituted quaterphenyl group, a substituted or unsubstituted fluorenyl group, a substituted or unsubstituted naphthyl group, a substituted or unsubstituted phenanthryl group, a substituted or unsubstituted dibenzofuran group, or a substituted or unsubstituted dibenzothiophene group. The light emitting element including the amine compound of the present disclosure in the emission layer satisfies the condition in which Ar₁ is not a substituted or unsubstituted carbazole group, and may exhibit an effect of reducing the driving voltage.

In an embodiment, An may include at least one selected from among Substituent SA1 to Substituent SA26 disclosed in Substituent Group SA. However, the embodiment of Ar₁ is not limited to Substituent SA1 to Substituent SA26.

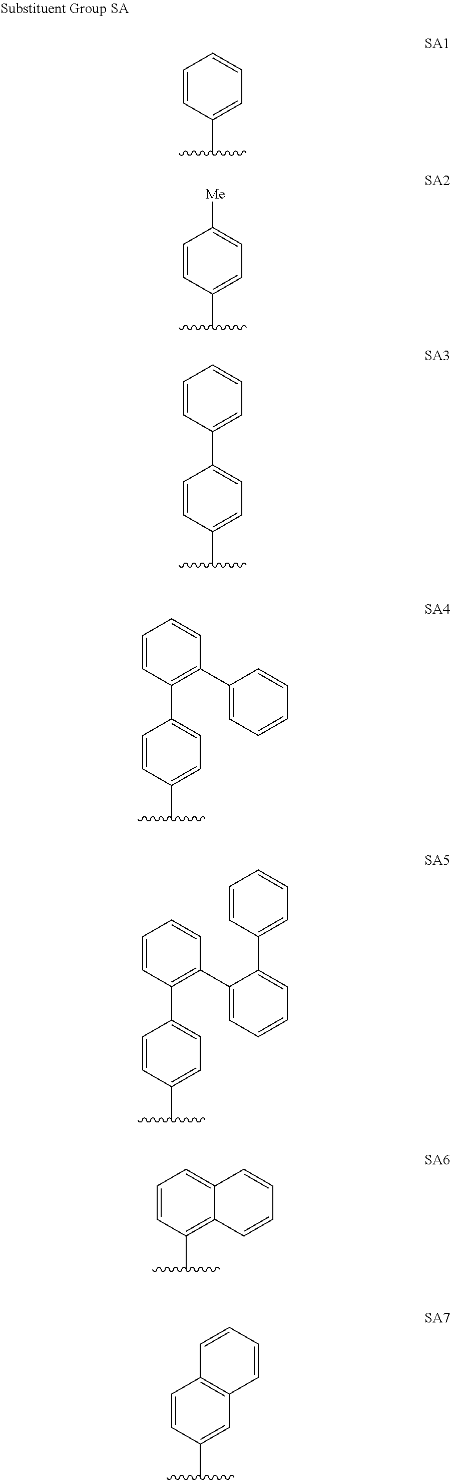

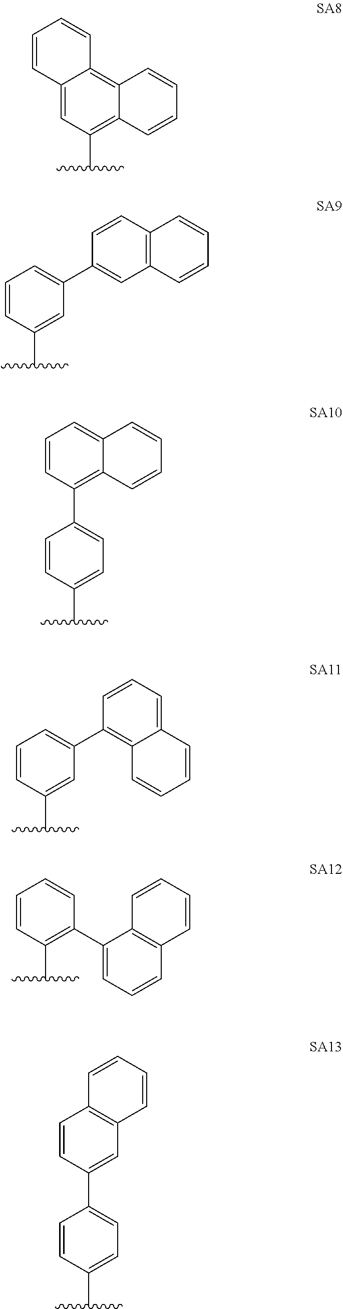 [SMILES: *c1cc2ccccc2c2ccccc12 ; *c1cccc(-c2ccc3ccccc3c2)c1 ; *c1ccc(-c2cccc3ccccc23)cc1 ; *c1cccc(-c2cccc3ccccc23)c1 ; *c1ccccc1-c1cccc2ccccc12 ; *c1ccc(-c2ccc3ccccc3c2)cc1]

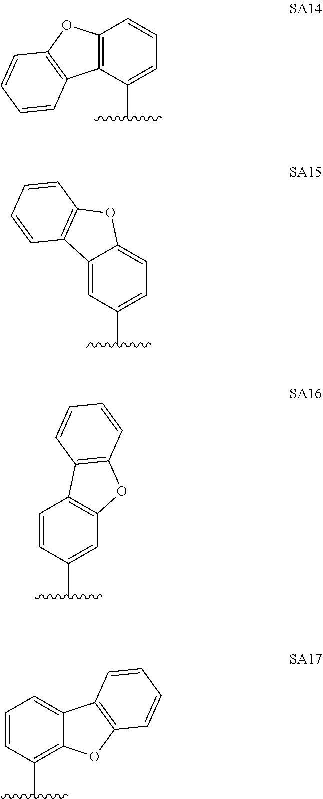 [SMILES: *c1cccc2oc3ccccc3c12 ; *c1ccc2oc3ccccc3c2c1 ; *c1ccc2c(c1)oc1ccccc12 ; *c1cccc2c1oc1ccccc12]

L may be a substituted or unsubstituted arylene group having 6 to 50 ring-forming carbon atoms, or a substituted or unsubstituted heteroarylene group having 2 to 50 ring-forming carbon atoms. For example, L may be a substituted or unsubstituted phenylene group, a substituted or unsubstituted biphenylene group, or a substituted or unsubstituted divalent dibenzofuran group.

In an embodiment, L may include at least one selected from among Substituent SL1 to Substituent SL5 disclosed in Substituent Group SL. However, the embodiment of L is not limited to Substituent SL1 to Substituent SL5.

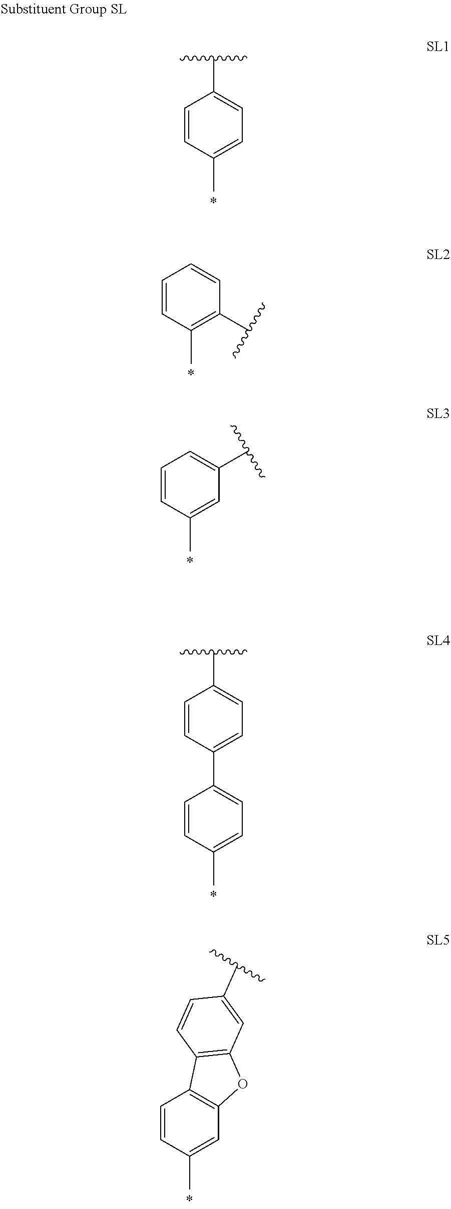

R₁ may be a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or may be bonded to an adjacent group to form a ring. For example, R₁ may be a hydrogen atom.

x1 is an integer from 0 to 5. For example, x1 may be 0. The embodiment in which x1 is 0 may be the same as the embodiment in which x1 is 5 and R₁ is a hydrogen atom. When x1 is 0, the amine compound represented by Formula 1 may not be substituted with R₁.

W₁ and W₂ may each independently be CRₐ or a carbon atom bonded to the nitrogen atom in Formula 1. For example, W₁ and W₂ may each independently be CRₐ or the nitrogen atom of the amine compound may be linked to any one selected from among W₁ and W₂.

However, when R₄ in Formula 2, which will be described in more detail, does not form a ring with an adjacent group, each of W₁ and W₂ in Formula 1 is CRₐ. For example, when R₄ in Formula 2, which will be described in more detail, does not form a ring with an adjacent group and FF has a substituted or unsubstituted naphthyl group structure, the nitrogen atom of the amine compound is not linked to W₁ and W₂. W₁ and W₂ may be carbon atoms corresponding to the third and sixth positions of the carbazole group, respectively.

For example, when R₄ in Formula 2 does not form a ring with an adjacent group, and thus FF has a substituted or unsubstituted naphthyl group structure, the nitrogen atom in Formula 1 may be linked to the second or fourth position of the carbazole group.

For example, when R₄ in Formula 2 does not form a ring with an adjacent group, and thus FF has a substituted or unsubstituted phenanthryl group structure, the nitrogen atom in Formula 1 may be linked to the second, third, or fourth position of the carbazole group. This is because when FF has a phenanthryl group structure, electron delocalization and resonance structure characteristics are enhanced compared with the embodiment of FF having a naphthyl group structure, and thus electronic effects between FF and the carbazole group may occur regardless of the linkage position of the nitrogen atom and the carbazole group in Formula 1.

Rₐ may be a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or may be bonded to an adjacent group to form a ring. For example, Rₐ may be a hydrogen atom.

FF is represented by Formula 2. In Formula 2, “—*” is a position linked to L.

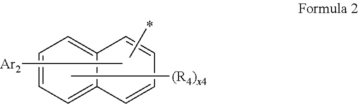

In Formula 2, Ar₂ may be a substituted or unsubstituted aryl group having 6 to 50 ring-forming carbon atoms, and the embodiment in which Ar₂ is an aryl group substituted with an amine group is excluded. For example, Ar₂ may be a substituted or unsubstituted phenyl group, and specifically, Ar₂ may be an unsubstituted phenyl group. However, embodiments are not limited thereto.

R₄ may be a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or may be bonded to an adjacent group to form a ring. For example, R₄ may be a hydrogen atom, or may be bonded to an adjacent group to form a phenanthryl group.

In some embodiments, when R₄ does not form a ring with an adjacent group, each of W₁ and W₂ in Formula 1 is CRₐ. The same as described in Formula 1 may be applied to this.

x4 is an integer from 0 to 6. For example, x4 may be 0 or 2. The embodiment in which x4 is 0 may be the same as the embodiment in which x4 is 6 and R₄s are hydrogen atoms. When x4 is 0, FF represented by Formula 2 may not be substituted with R₄. The embodiment in which x4 is 2 may be the embodiment in which R₄s are bonded to each other to form a ring, for example, two R₄s may be bonded to each other to form a phenanthryl group.

However, when Formula 2 is represented by

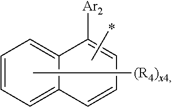

R₄ does not form a ring with an adjacent group, and Ar₁ is a substituted or unsubstituted fluorene group, and the embodiment in which the nitrogen atom in Formula 1 is linked to the second position of the substituted or unsubstituted fluorene group, Ar₁, is excluded. In this embodiment, in the amine compound, the carbazole group, the naphthyl group substituted with an aryl group, and the fluorene group are closer to each other, and thus the molecule is further twisted, and the resistance to redox is reduced, and thus there may occur a limitation in increasing driving voltage.

In an embodiment, FF represented by Formula 2 may be represented by Formula 2a:

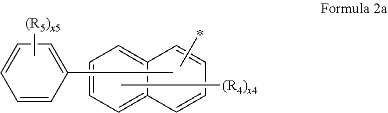

Formula 2a is the embodiment in which Are in Formula 2 is a substituted or unsubstituted phenyl group. For example, FF in Formula 1 may be a naphthyl group substituted with a phenyl group or a phenanthryl group substituted with a phenyl group. However, embodiments are not limited thereto.

In Formula 2a, R₅ may be a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or may be bonded to an adjacent group to form a ring. However, the embodiment in which R₅ is an amine group is excluded. For example, R₅ may be a hydrogen atom.

x5 may be an integer from 0 to 5. For example, x5 may be 0. The embodiment in which x5 is 0 may be the same as the embodiment in which x5 is 5 and R₅s are hydrogen atoms. When x5 is 0, FF represented by Formula 2a may not be substituted with R₅.

FF represented by Formula 2a may further include a substituent represented by R₄ in addition to a substituted or unsubstituted phenyl group.

R₄ and x4 are the same as defined in Formula 2.

In embodiment, FF represented by Formula 2 may be represented by any one selected from among Formula 2-1 to Formula 2-5:

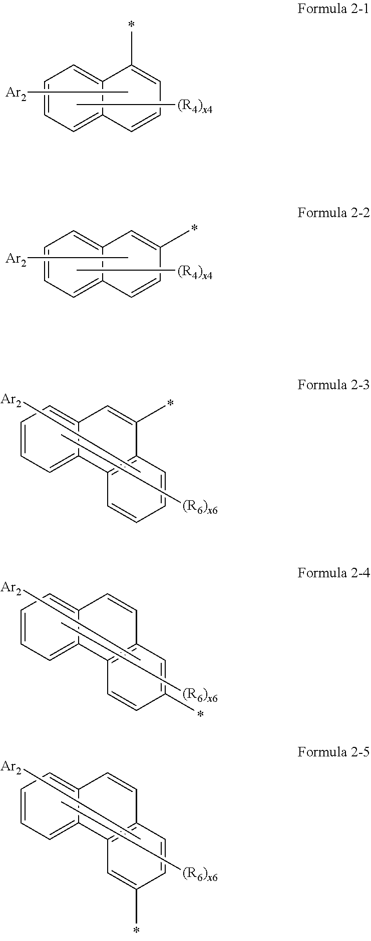

Formula 2-1 to Formula 2-5 are the embodiments in which the position of “—*” is specified in Formula 2. In some embodiments, Formula 2-3 and Formula 2-5 are the embodiments in which R₄ in Formula 2 is specified as R₆. For example, FF in Formula 1 may be a substituted or unsubstituted naphthyl group, or a substituted or unsubstituted phenanthryl group. However, embodiments are not limited thereto.

In Formula 2-1 to Formula 2-5, R₆ may be a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or may be bonded to an adjacent group to form a ring. For example, R₆ is a hydrogen atom.

x6 may be an integer from 0 to 8. For example, x6 may be 0. The embodiment in which x6 is 0 may be the same as the embodiment in which x6 is 8 and R₆ is a hydrogen atom. When x6 is 0, FF represented by Formula 2-3, Formula 2-4, or Formula 2-5 may not be substituted with R₆.

Ar₂, R₄, and x4 are the same as defined in Formula 2.

In an embodiment, FF may be represented by any one selected from among Formula 2-3a, Formula 2-4a, and Formula 2-5a:

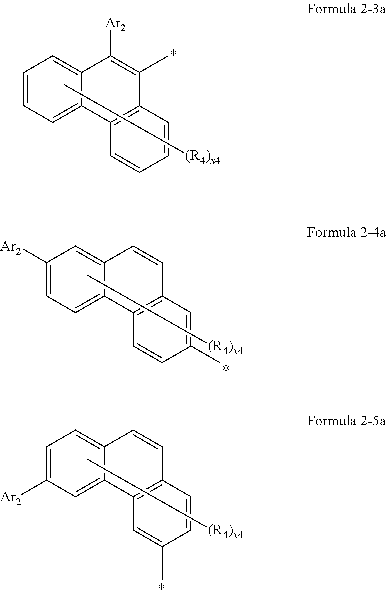

Formula 2-3a, Formula 2-4a, and Formula 2-5a are the embodiments in which the position linked to Ar₂ is specified in Formula 2-3, Formula 2-4, and Formula 2-5, respectively. However, embodiments are not limited thereto.

In Formula 2-3a to Formula 2-5a, Ar₂, R₄, and x4 are the same as defined in Formula 2.

In an embodiment, the amine compound represented by Formula 1 may be represented by any one selected from among Formula 3-1 to Formula 3-3:

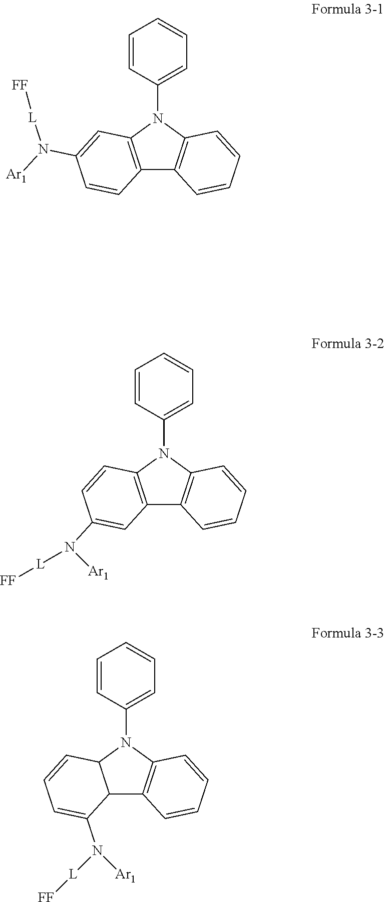

Formula 3-1 to Formula 3-3 are the embodiments in which the position in which a 9-phenyl-9H-carbazole group in Formula 1 is linked to the nitrogen atom of the amine compound is specified.

Formula 3-1 is the embodiment in which the nitrogen atom of the amine compound is linked to the second position of the 9-phenyl-9H-carbazole group.

Formula 3-2 is the embodiment in which the nitrogen atom of the amine compound is linked to the third position of the 9-phenyl-9H-carbazole group. In some embodiments, as described in Formula 1, when R₄ does not form a ring with an adjacent group, each of W₁ and W₂ in Formula 1 is CRₐ. Formula 3-2 is a structure in which the nitrogen atom is linked to W₁ in Formula 1, that is, R₄ in Formula 2 forms a ring with an adjacent group. For example, Formula 3-2 may be the embodiment in which R₄ in Formula 2 forms a ring with an adjacent group to form a phenanthryl group, and W₁ in Formula 1 is linked to the nitrogen atom.

Formula 3-3 is the embodiment in which the nitrogen atom of the amine compound is linked to the fourth position of the 9-phenyl-9H-carbazole group.

In Formula 3-1 to Formula 3-3, L, Ar₁, and FF are the same as defined in Formula 1.

The amine compound of the present disclosure may include an arylamine group in which Ar₁ and Ar₂ are linked to the central nitrogen atom. For example, the amine compound of the present disclosure may have an improvement in the ability to supply and transport holes due to the interaction between the arylamine group and the carbazole group by directly linking the carbazole group (9-phenyl-9H-carbazole group) to the nitrogen atom linked to a substituted or unsubstituted naphthyl group.

The amine compound represented by Formula 1 of an embodiment may be represented by one selected from among the compounds of Compound Group 1. The hole transport region HTR of the light emitting element ED of an embodiment may include at least one selected from among the amine compounds disclosed in Compound Group 1.

In some embodiments, “FF—*” in Compound Group 1 is linked to “*—L.” Each of

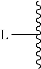

and

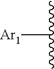

is linked to

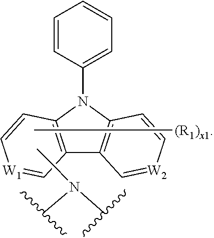

Compound Group 1

| Divi- sion | 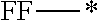 | 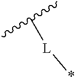 | 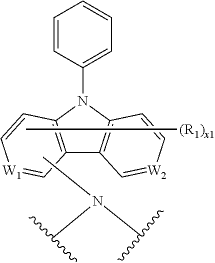 | 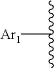 |
|---|---|---|---|---|
| A1 | 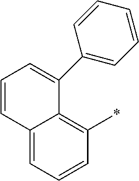 [SMILES: *c1cccc2cccc(-c3ccccc3)c12] | 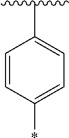 [SMILES: *c1ccc(*)cc1] | 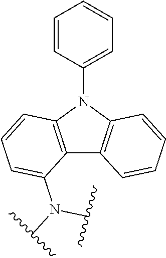 [SMILES: *N(*)c1cccc2c1c1ccccc1n2-c1ccccc1] | 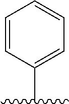 [SMILES: *c1ccccc1] |
| A2 | 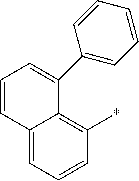 [SMILES: *c1cccc2cccc(-c3ccccc3)c12] | 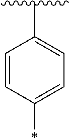 [SMILES: *c1ccc(*)cc1] | 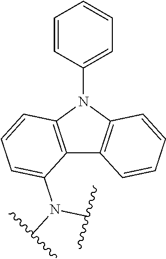 [SMILES: *N(*)c1cccc2c1c1ccccc1n2-c1ccccc1] | 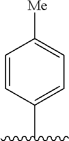 [SMILES: *c1ccc(C)cc1] |
| A3 | 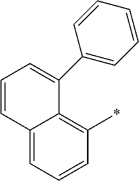 [SMILES: *c1cccc2cccc(-c3ccccc3)c12] | 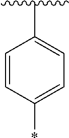 [SMILES: *c1ccc(*)cc1] | 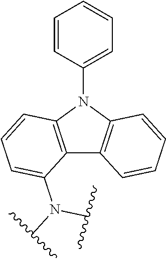 [SMILES: *N(*)c1cccc2c1c1ccccc1n2-c1ccccc1] | 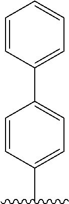 [SMILES: *c1ccc(-c2ccccc2)cc1] |
| A4 | 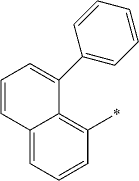 [SMILES: *c1cccc2cccc(-c3ccccc3)c12] | 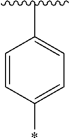 [SMILES: *c1ccc(*)cc1] | 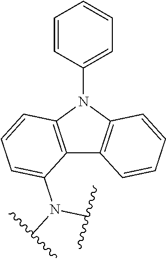 [SMILES: *N(*)c1cccc2c1c1ccccc1n2-c1ccccc1] | 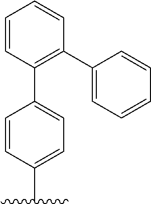 [SMILES: *c1ccc(-c2ccccc2-c2ccccc2)cc1] |
| A5 | 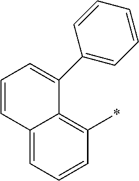 [SMILES: *c1cccc2cccc(-c3ccccc3)c12] | 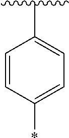 [SMILES: *c1ccc(*)cc1] | 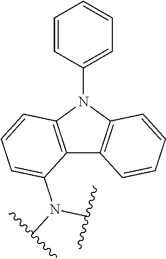 [SMILES: *N(*)c1cccc2c1c1ccccc1n2-c1ccccc1] | 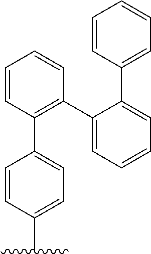 [SMILES: *c1ccc(-c2ccccc2-c2ccccc2-c2ccccc2)cc1] |
| A6 | 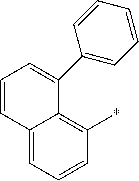 [SMILES: *c1cccc2cccc(-c3ccccc3)c12] | 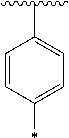 [SMILES: *c1ccc(*)cc1] | 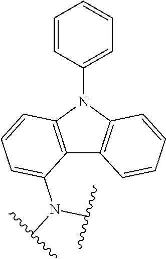 [SMILES: *N(*)c1cccc2c1c1ccccc1n2-c1ccccc1] | 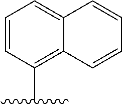 [SMILES: *c1cccc2ccccc12] |
| A7 | 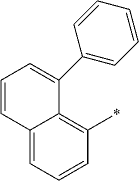 [SMILES: *c1cccc2cccc(-c3ccccc3)c12] | 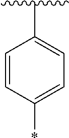 [SMILES: *c1ccc(*)cc1] | 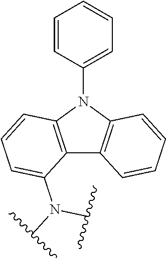 [SMILES: *N(*)c1cccc2c1c1ccccc1n2-c1ccccc1] | 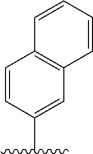 [SMILES: *c1ccc2ccccc2c1] |
| A8 | 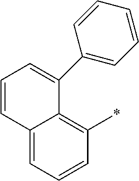 [SMILES: *c1cccc2cccc(-c3ccccc3)c12] | 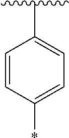 [SMILES: *c1ccc(*)cc1] | 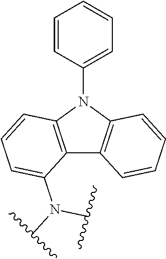 [SMILES: *N(*)c1cccc2c1c1ccccc1n2-c1ccccc1] | 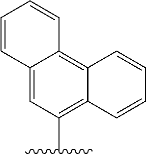 [SMILES: *c1cc2ccccc2c2ccccc12] |
| A9 | 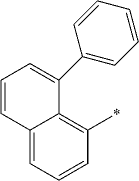 [SMILES: *c1cccc2cccc(-c3ccccc3)c12] | 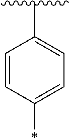 [SMILES: *c1ccc(*)cc1] | 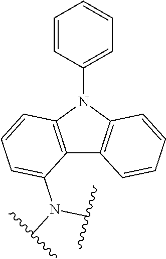 [SMILES: *N(*)c1cccc2c1c1ccccc1n2-c1ccccc1] | 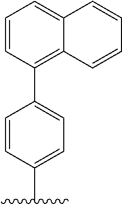 [SMILES: *c1ccc(-c2cccc3ccccc23)cc1] |
| A10 | 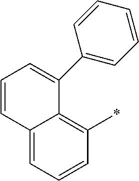 [SMILES: *c1cccc2cccc(-c3ccccc3)c12] | 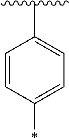 [SMILES: *c1ccc(*)cc1] | 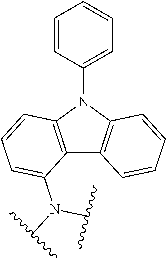 [SMILES: *N(*)c1cccc2c1c1ccccc1n2-c1ccccc1] | 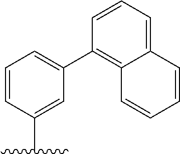 [SMILES: *c1cccc(-c2cccc3ccccc23)c1] |
| A11 | 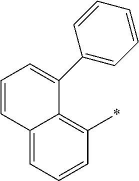 [SMILES: *c1cccc2cccc(-c3ccccc3)c12] | 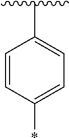 [SMILES: *c1ccc(*)cc1] | 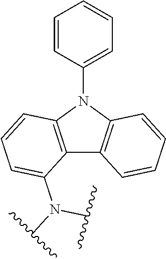 [SMILES: *N(*)c1cccc2c1c1ccccc1n2-c1ccccc1] | 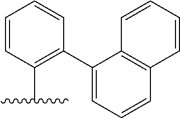 [SMILES: *c1ccccc1-c1cccc2ccccc12] |
| A12 | 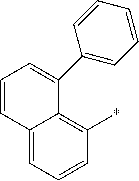 [SMILES: *c1cccc2cccc(-c3ccccc3)c12] | 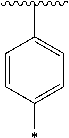 [SMILES: *c1ccc(*)cc1] | 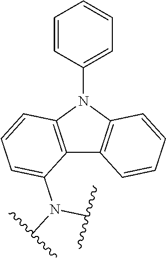 [SMILES: *N(*)c1cccc2c1c1ccccc1n2-c1ccccc1] | 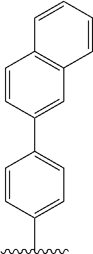 [SMILES: *c1ccc(-c2ccc3ccccc3c2)cc1] |
| A13 | 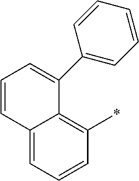 [SMILES: *c1cccc2cccc(-c3ccccc3)c12] | 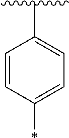 [SMILES: *c1ccc(*)cc1] | 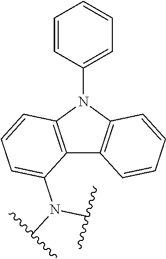 [SMILES: *N(*)c1cccc2c1c1ccccc1n2-c1ccccc1] | 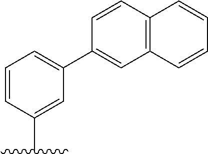 [SMILES: *c1cccc(-c2ccc3ccccc3c2)c1] |
| A14 | 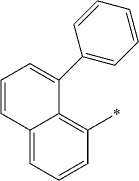 [SMILES: *c1cccc2cccc(-c3ccccc3)c12] | 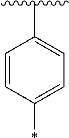 [SMILES: *c1ccc(*)cc1] | 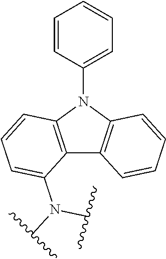 [SMILES: *N(*)c1cccc2c1c1ccccc1n2-c1ccccc1] | 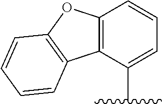 [SMILES: *c1cccc2oc3ccccc3c12] |
| A15 | 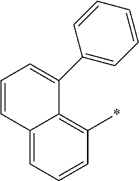 [SMILES: *c1cccc2cccc(-c3ccccc3)c12] | 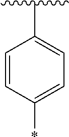 [SMILES: *c1ccc(*)cc1] | 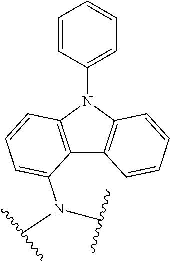 [SMILES: *N(*)c1cccc2c1c1ccccc1n2-c1ccccc1] | 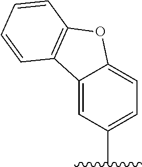 [SMILES: *c1ccc2oc3ccccc3c2c1] |
| A16 | 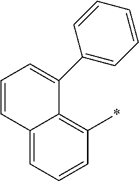 [SMILES: *c1cccc2cccc(-c3ccccc3)c12] | 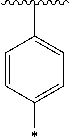 [SMILES: *c1ccc(*)cc1] | 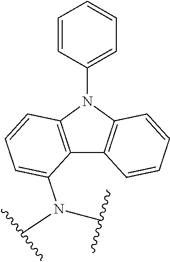 [SMILES: *N(*)c1cccc2c1c1ccccc1n2-c1ccccc1] | 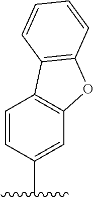 [SMILES: *c1ccc2c(c1)oc1ccccc12] |
| A17 | 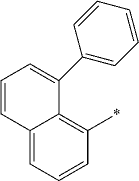 [SMILES: *c1cccc2cccc(-c3ccccc3)c12] | 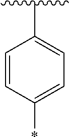 [SMILES: *c1ccc(*)cc1] | 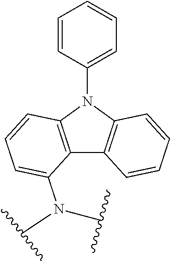 [SMILES: *N(*)c1cccc2c1c1ccccc1n2-c1ccccc1] | 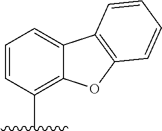 [SMILES: *c1cccc2c1oc1ccccc12] |
| A18 | 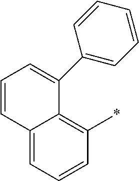 [SMILES: *c1cccc2cccc(-c3ccccc3)c12] | 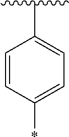 [SMILES: *c1ccc(*)cc1] | 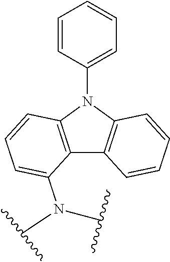 [SMILES: *N(*)c1cccc2c1c1ccccc1n2-c1ccccc1] | 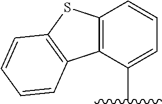 [SMILES: *c1cccc2sc3ccccc3c12] |
| A19 | 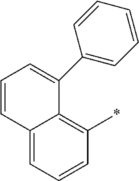 [SMILES: *c1cccc2cccc(-c3ccccc3)c12] | 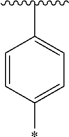 [SMILES: *c1ccc(*)cc1] | 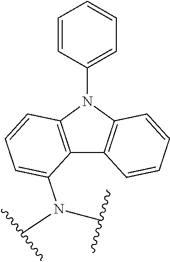 [SMILES: *N(*)c1cccc2c1c1ccccc1n2-c1ccccc1] | 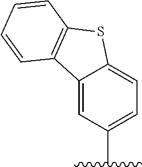 [SMILES: *c1ccc2sc3ccccc3c2c1] |
| A20 | 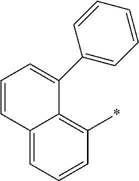 [SMILES: *c1cccc2cccc(-c3ccccc3)c12] | 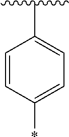 [SMILES: *c1ccc(*)cc1] | 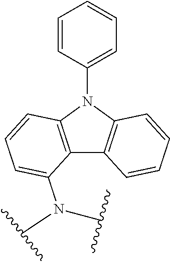 [SMILES: *N(*)c1cccc2c1c1ccccc1n2-c1ccccc1] | 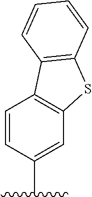 [SMILES: *c1ccc2c(c1)sc1ccccc12] |
| A21 | 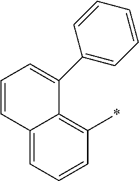 [SMILES: *c1cccc2cccc(-c3ccccc3)c12] | 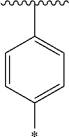 [SMILES: *c1ccc(*)cc1] | 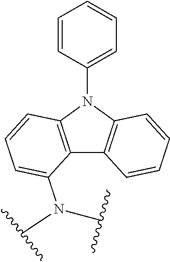 [SMILES: *N(*)c1cccc2c1c1ccccc1n2-c1ccccc1] | 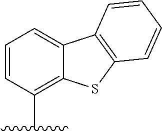 [SMILES: *c1cccc2c1sc1ccccc12] |
| A22 | 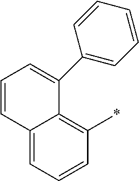 [SMILES: *c1cccc2cccc(-c3ccccc3)c12] | 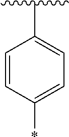 [SMILES: *c1ccc(*)cc1] | 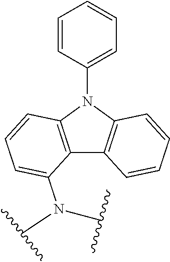 [SMILES: *N(*)c1cccc2c1c1ccccc1n2-c1ccccc1] | 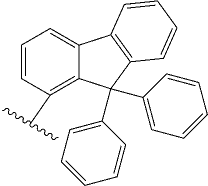 [SMILES: *c1cccc2c1C(c1ccccc1)(c1ccccc1)c1ccccc1-2] |
| A23 | 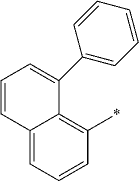 [SMILES: *c1cccc2cccc(-c3ccccc3)c12] | 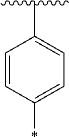 [SMILES: *c1ccc(*)cc1] | 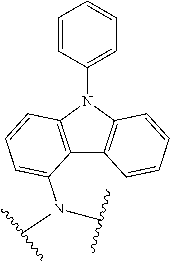 [SMILES: *N(*)c1cccc2c1c1ccccc1n2-c1ccccc1] | 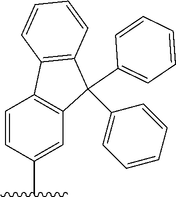 [SMILES: *c1ccc2c(c1)C(c1ccccc1)(c1ccccc1)c1ccccc1-2] |
| A24 | 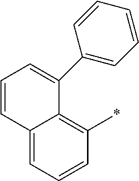 [SMILES: *c1cccc2cccc(-c3ccccc3)c12] | 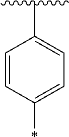 [SMILES: *c1ccc(*)cc1] | 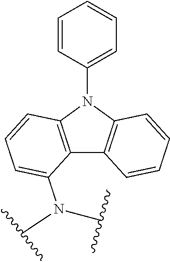 [SMILES: *N(*)c1cccc2c1c1ccccc1n2-c1ccccc1] | 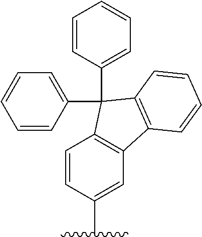 [SMILES: *c1ccc2c(c1)-c1ccccc1C2(c1ccccc1)c1ccccc1] |
| A25 | 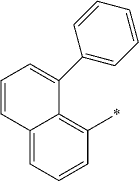 [SMILES: *c1cccc2cccc(-c3ccccc3)c12] | 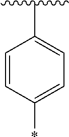 [SMILES: *c1ccc(*)cc1] | 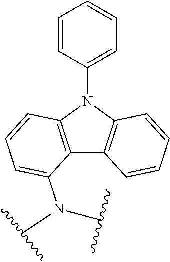 [SMILES: *N(*)c1cccc2c1c1ccccc1n2-c1ccccc1] | 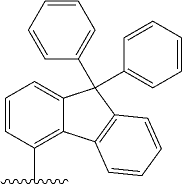 [SMILES: *c1cccc2c1-c1ccccc1C2(c1ccccc1)c1ccccc1] |
| A26 | 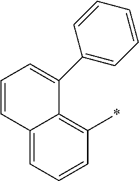 [SMILES: *c1cccc2cccc(-c3ccccc3)c12] | 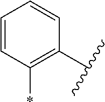 [SMILES: *c1ccccc1*] | 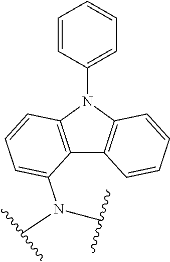 [SMILES: *N(*)c1cccc2c1c1ccccc1n2-c1ccccc1] | 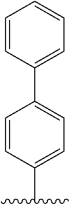 [SMILES: *c1ccc(-c2ccccc2)cc1] |
| A27 | 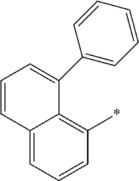 [SMILES: *c1cccc2cccc(-c3ccccc3)c12] | 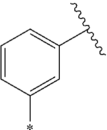 [SMILES: *c1cccc(*)c1] | 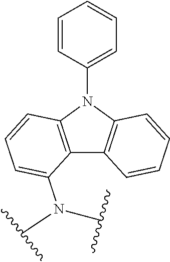 [SMILES: *N(*)c1cccc2c1c1ccccc1n2-c1ccccc1] | 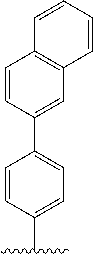 [SMILES: *c1ccc(-c2ccc3ccccc3c2)cc1] |
| A28 | 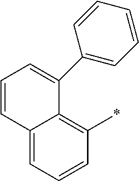 [SMILES: *c1cccc2cccc(-c3ccccc3)c12] | 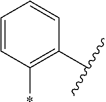 [SMILES: *c1ccccc1*] | 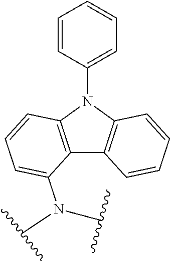 [SMILES: *N(*)c1cccc2c1c1ccccc1n2-c1ccccc1] | 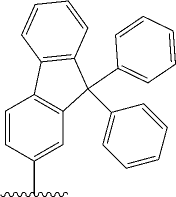 [SMILES: *c1ccc2c(c1)C(c1ccccc1)(c1ccccc1)c1ccccc1-2] |
| A29 | 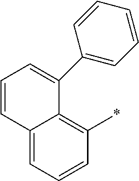 [SMILES: *c1cccc2cccc(-c3ccccc3)c12] | 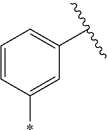 [SMILES: *c1cccc(*)c1] | 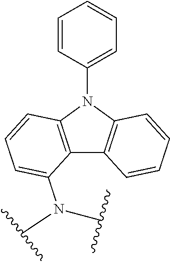 [SMILES: *N(*)c1cccc2c1c1ccccc1n2-c1ccccc1] | 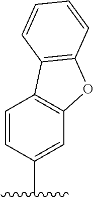 [SMILES: *c1ccc2c(c1)oc1ccccc12] |
| A30 | 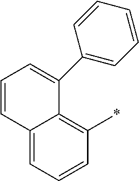 [SMILES: *c1cccc2cccc(-c3ccccc3)c12] | 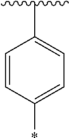 [SMILES: *c1ccc(*)cc1] | 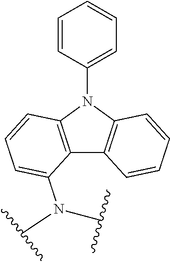 [SMILES: *N(*)c1cccc2c1c1ccccc1n2-c1ccccc1] | 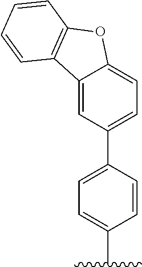 [SMILES: *c1ccc(-c2ccc3oc4ccccc4c3c2)cc1] |
| A31 | 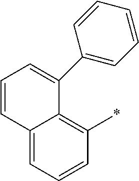 [SMILES: *c1cccc2cccc(-c3ccccc3)c12] | 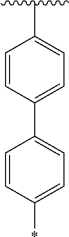 [SMILES: *c1ccc(-c2ccc(*)cc2)cc1] | 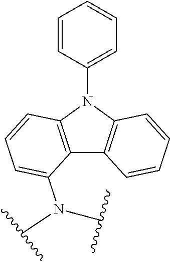 [SMILES: *N(*)c1cccc2c1c1ccccc1n2-c1ccccc1] | 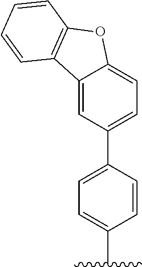 [SMILES: *c1ccc(-c2ccc3oc4ccccc4c3c2)cc1] |
| A32 | 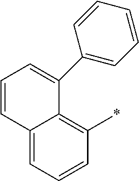 [SMILES: *c1cccc2cccc(-c3ccccc3)c12] | 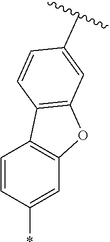 [SMILES: *c1ccc2c(c1)oc1cc(*)ccc12] | 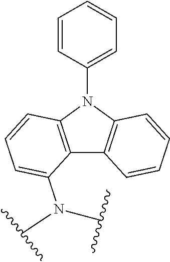 [SMILES: *N(*)c1cccc2c1c1ccccc1n2-c1ccccc1] | 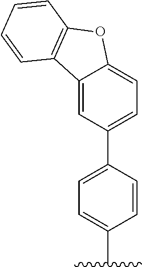 [SMILES: *c1ccc(-c2ccc3oc4ccccc4c3c2)cc1] |
| A33 | 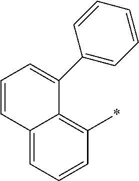 [SMILES: *c1cccc2cccc(-c3ccccc3)c12] | 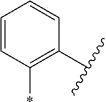 [SMILES: *c1ccccc1*] | 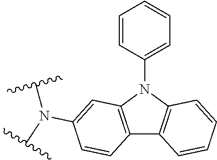 [SMILES: *N(*)c1ccc2c3ccccc3n(-c3ccccc3)c2c1] | 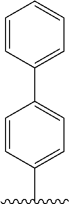 [SMILES: *c1ccc(-c2ccccc2)cc1] |
| A34 | 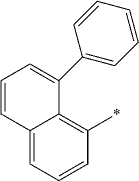 [SMILES: *c1cccc2cccc(-c3ccccc3)c12] | 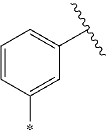 [SMILES: *c1cccc(*)c1] | 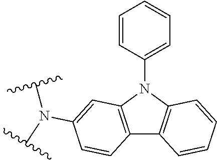 [SMILES: *N(*)c1ccc2c3ccccc3n(-c3ccccc3)c2c1] | 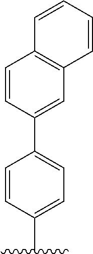 [SMILES: *c1ccc(-c2ccc3ccccc3c2)cc1] |
| A35 | 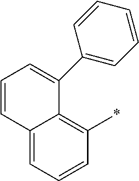 [SMILES: *c1cccc2cccc(-c3ccccc3)c12] | 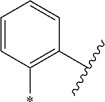 [SMILES: *c1ccccc1*] | 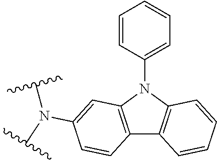 [SMILES: *N(*)c1ccc2c3ccccc3n(-c3ccccc3)c2c1] | 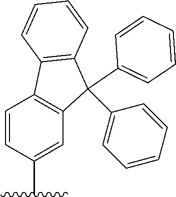 [SMILES: *c1ccc2c(c1)C(c1ccccc1)(c1ccccc1)c1ccccc1-2] |
| A36 | 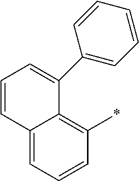 [SMILES: *c1cccc2cccc(-c3ccccc3)c12] | 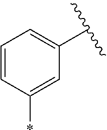 [SMILES: *c1cccc(*)c1] | 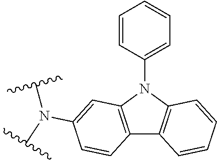 [SMILES: *N(*)c1ccc2c3ccccc3n(-c3ccccc3)c2c1] | 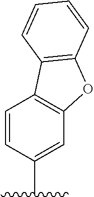 [SMILES: *c1ccc2c(c1)oc1ccccc12] |
| A37 | 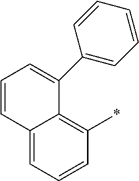 [SMILES: *c1cccc2cccc(-c3ccccc3)c12] | 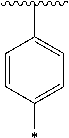 [SMILES: *c1ccc(*)cc1] | 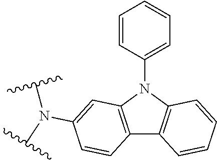 [SMILES: *N(*)c1ccc2c3ccccc3n(-c3ccccc3)c2c1] | 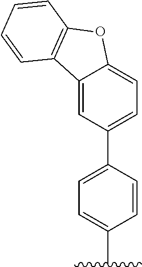 [SMILES: *c1ccc(-c2ccc3oc4ccccc4c3c2)cc1] |
| A38 | 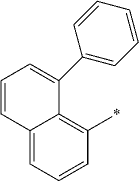 [SMILES: *c1cccc2cccc(-c3ccccc3)c12] | 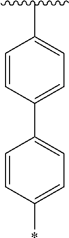 [SMILES: *c1ccc(-c2ccc(*)cc2)cc1] | 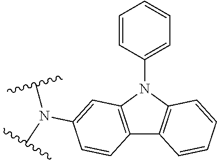 [SMILES: *N(*)c1ccc2c3ccccc3n(-c3ccccc3)c2c1] | 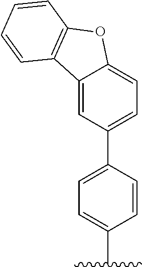 [SMILES: *c1ccc(-c2ccc3oc4ccccc4c3c2)cc1] |
| A39 | 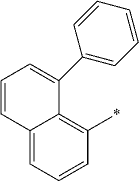 [SMILES: *c1cccc2cccc(-c3ccccc3)c12] | 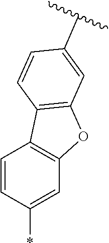 [SMILES: *c1ccc2c(c1)oc1cc(*)ccc12] | 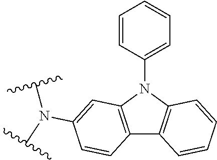 [SMILES: *N(*)c1ccc2c3ccccc3n(-c3ccccc3)c2c1] | 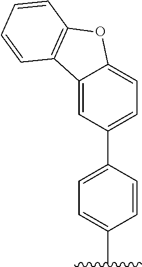 [SMILES: *c1ccc(-c2ccc3oc4ccccc4c3c2)cc1] |
| B1 | 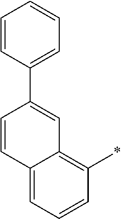 [SMILES: *c1cccc2ccc(-c3ccccc3)cc12] | 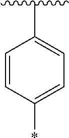 [SMILES: *c1ccc(*)cc1] | 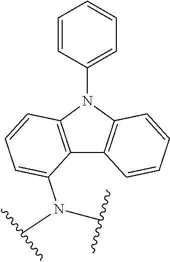 [SMILES: *N(*)c1cccc2c1c1ccccc1n2-c1ccccc1] | 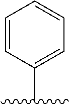 [SMILES: *c1ccccc1] |
| B2 | 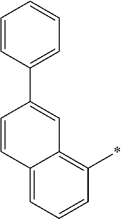 [SMILES: *c1cccc2ccc(-c3ccccc3)cc12] | 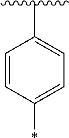 [SMILES: *c1ccc(*)cc1] | 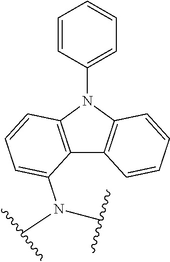 [SMILES: *N(*)c1cccc2c1c1ccccc1n2-c1ccccc1] | 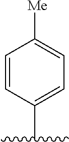 [SMILES: *c1ccc(C)cc1] |
| B3 | 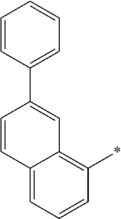 [SMILES: *c1cccc2ccc(-c3ccccc3)cc12] | 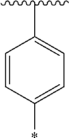 [SMILES: *c1ccc(*)cc1] | 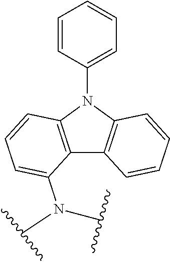 [SMILES: *N(*)c1cccc2c1c1ccccc1n2-c1ccccc1] | 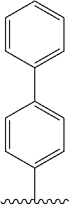 [SMILES: *c1ccc(-c2ccccc2)cc1] |
| B4 | 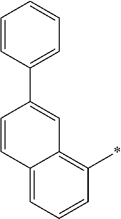 [SMILES: *c1cccc2ccc(-c3ccccc3)cc12] | 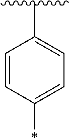 [SMILES: *c1ccc(*)cc1] | 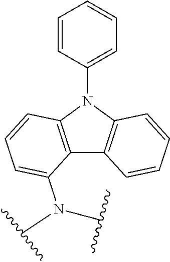 [SMILES: *N(*)c1cccc2c1c1ccccc1n2-c1ccccc1] | 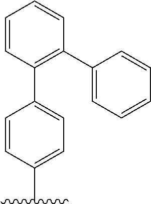 [SMILES: *c1ccc(-c2ccccc2-c2ccccc2)cc1] |
| B5 | 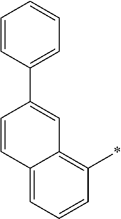 [SMILES: *c1cccc2ccc(-c3ccccc3)cc12] | 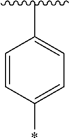 [SMILES: *c1ccc(*)cc1] | 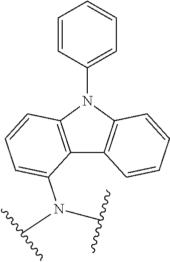 [SMILES: *N(*)c1cccc2c1c1ccccc1n2-c1ccccc1] | 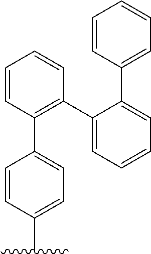 [SMILES: *c1ccc(-c2ccccc2-c2ccccc2-c2ccccc2)cc1] |
| B6 | 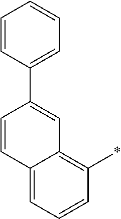 [SMILES: *c1cccc2ccc(-c3ccccc3)cc12] | 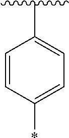 [SMILES: *c1ccc(*)cc1] | 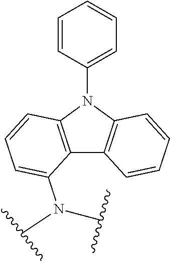 [SMILES: *N(*)c1cccc2c1c1ccccc1n2-c1ccccc1] | 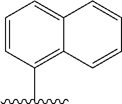 [SMILES: *c1cccc2ccccc12] |
| B7 | 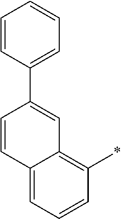 [SMILES: *c1cccc2ccc(-c3ccccc3)cc12] | 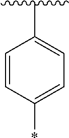 [SMILES: *c1ccc(*)cc1] | 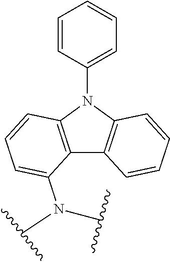 [SMILES: *N(*)c1cccc2c1c1ccccc1n2-c1ccccc1] | 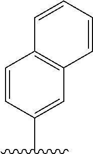 [SMILES: *c1ccc2ccccc2c1] |
| B8 | 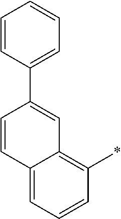 [SMILES: *c1cccc2ccc(-c3ccccc3)cc12] | 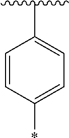 [SMILES: *c1ccc(*)cc1] | 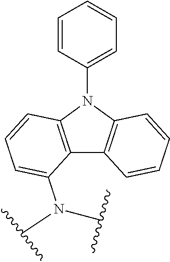 [SMILES: *N(*)c1cccc2c1c1ccccc1n2-c1ccccc1] | 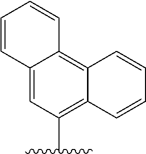 [SMILES: *c1cc2ccccc2c2ccccc12] |
| B9 | 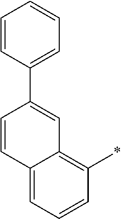 [SMILES: *c1cccc2ccc(-c3ccccc3)cc12] | 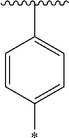 [SMILES: *c1ccc(*)cc1] | 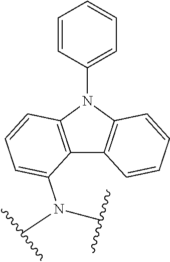 [SMILES: *N(*)c1cccc2c1c1ccccc1n2-c1ccccc1] | 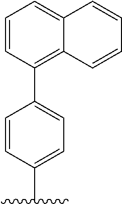 [SMILES: *c1ccc(-c2cccc3ccccc23)cc1] |
| B10 | 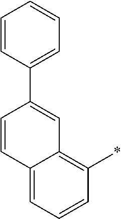 [SMILES: *c1cccc2ccc(-c3ccccc3)cc12] | 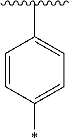 [SMILES: *c1ccc(*)cc1] | 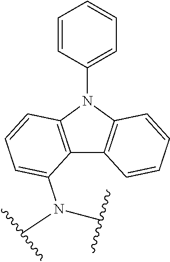 [SMILES: *N(*)c1cccc2c1c1ccccc1n2-c1ccccc1] | 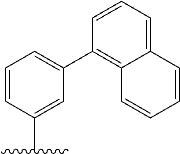 [SMILES: *c1cccc(-c2cccc3ccccc23)c1] |
| B11 | 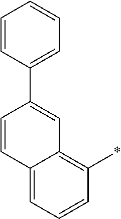 [SMILES: *c1cccc2ccc(-c3ccccc3)cc12] | 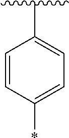 [SMILES: *c1ccc(*)cc1] | 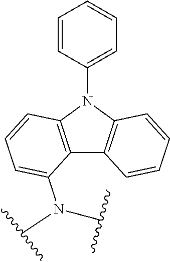 [SMILES: *N(*)c1cccc2c1c1ccccc1n2-c1ccccc1] | 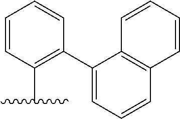 [SMILES: *c1ccccc1-c1cccc2ccccc12] |
| B12 | 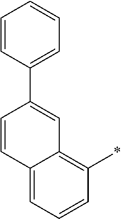 [SMILES: *c1cccc2ccc(-c3ccccc3)cc12] | 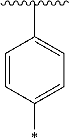 [SMILES: *c1ccc(*)cc1] | 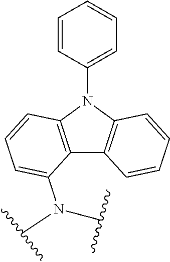 [SMILES: *N(*)c1cccc2c1c1ccccc1n2-c1ccccc1] | 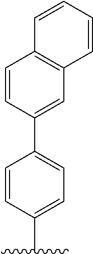 [SMILES: *c1ccc(-c2ccc3ccccc3c2)cc1] |
| B13 | 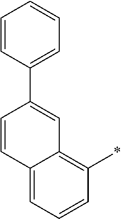 [SMILES: *c1cccc2ccc(-c3ccccc3)cc12] | 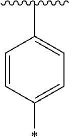 [SMILES: *c1ccc(*)cc1] | 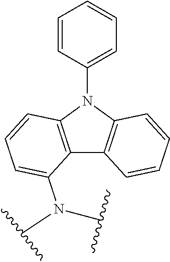 [SMILES: *N(*)c1cccc2c1c1ccccc1n2-c1ccccc1] | 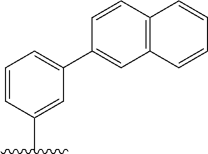 [SMILES: *c1cccc(-c2ccc3ccccc3c2)c1] |
| B14 | 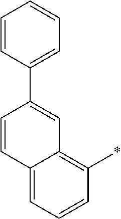 [SMILES: *c1cccc2ccc(-c3ccccc3)cc12] | 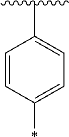 [SMILES: *c1ccc(*)cc1] | 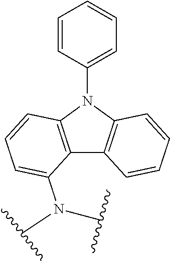 [SMILES: *N(*)c1cccc2c1c1ccccc1n2-c1ccccc1] | 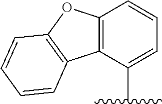 [SMILES: *c1cccc2oc3ccccc3c12] |
| B15 | 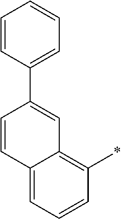 [SMILES: *c1cccc2ccc(-c3ccccc3)cc12] | 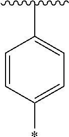 [SMILES: *c1ccc(*)cc1] | 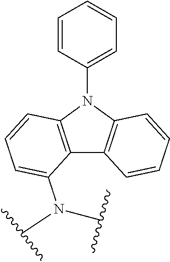 [SMILES: *N(*)c1cccc2c1c1ccccc1n2-c1ccccc1] | 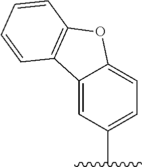 [SMILES: *c1ccc2oc3ccccc3c2c1] |
| B16 | 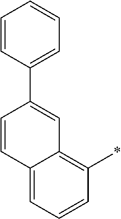 [SMILES: *c1cccc2ccc(-c3ccccc3)cc12] | 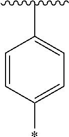 [SMILES: *c1ccc(*)cc1] | 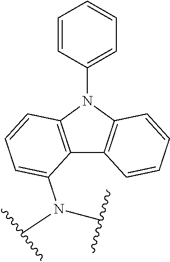 [SMILES: *N(*)c1cccc2c1c1ccccc1n2-c1ccccc1] | 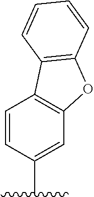 [SMILES: *c1ccc2c(c1)oc1ccccc12] |
| B17 | 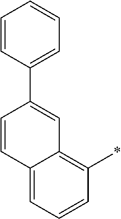 [SMILES: *c1cccc2ccc(-c3ccccc3)cc12] | 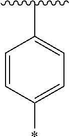 [SMILES: *c1ccc(*)cc1] | 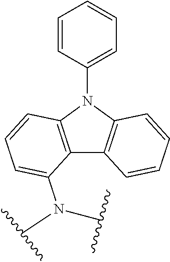 [SMILES: *N(*)c1cccc2c1c1ccccc1n2-c1ccccc1] | 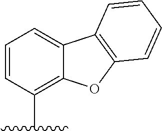 [SMILES: *c1cccc2c1oc1ccccc12] |
| B18 | 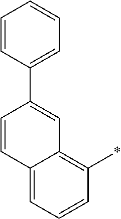 [SMILES: *c1cccc2ccc(-c3ccccc3)cc12] | 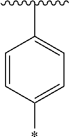 [SMILES: *c1ccc(*)cc1] | 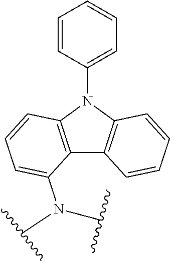 [SMILES: *N(*)c1cccc2c1c1ccccc1n2-c1ccccc1] | 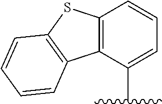 [SMILES: *c1cccc2sc3ccccc3c12] |
| B19 | 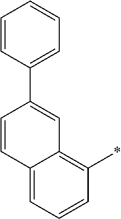 [SMILES: *c1cccc2ccc(-c3ccccc3)cc12] | 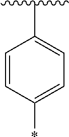 [SMILES: *c1ccc(*)cc1] | 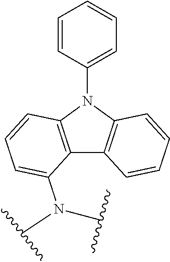 [SMILES: *N(*)c1cccc2c1c1ccccc1n2-c1ccccc1] | 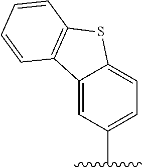 [SMILES: *c1ccc2sc3ccccc3c2c1] |
| B20 | 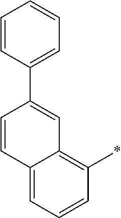 [SMILES: *c1cccc2ccc(-c3ccccc3)cc12] | 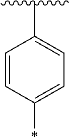 [SMILES: *c1ccc(*)cc1] | 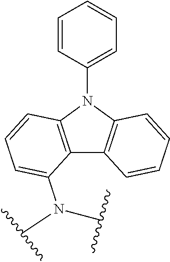 [SMILES: *N(*)c1cccc2c1c1ccccc1n2-c1ccccc1] | 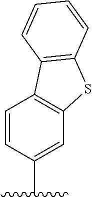 [SMILES: *c1ccc2c(c1)sc1ccccc12] |
| B21 | 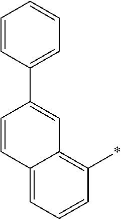 [SMILES: *c1cccc2ccc(-c3ccccc3)cc12] | 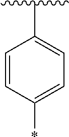 [SMILES: *c1ccc(*)cc1] | 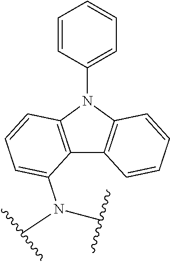 [SMILES: *N(*)c1cccc2c1c1ccccc1n2-c1ccccc1] | 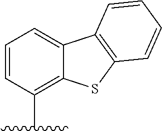 [SMILES: *c1cccc2c1sc1ccccc12] |
| B22 | 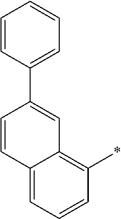 [SMILES: *c1cccc2ccc(-c3ccccc3)cc12] | 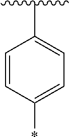 [SMILES: *c1ccc(*)cc1] | 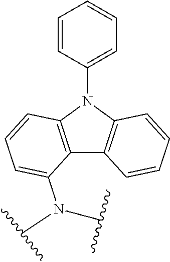 [SMILES: *N(*)c1cccc2c1c1ccccc1n2-c1ccccc1] | 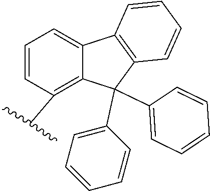 [SMILES: *c1cccc2c1C(c1ccccc1)(c1ccccc1)c1ccccc1-2] |
| B23 | 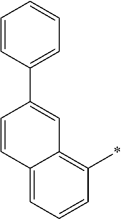 [SMILES: *c1cccc2ccc(-c3ccccc3)cc12] | 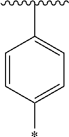 [SMILES: *c1ccc(*)cc1] | 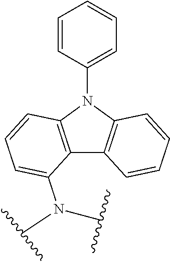 [SMILES: *N(*)c1cccc2c1c1ccccc1n2-c1ccccc1] | 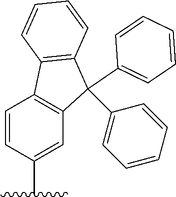 [SMILES: *c1ccc2c(c1)C(c1ccccc1)(c1ccccc1)c1ccccc1-2] |
| B24 | 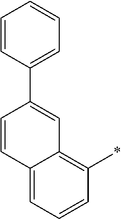 [SMILES: *c1cccc2ccc(-c3ccccc3)cc12] | 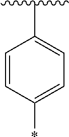 [SMILES: *c1ccc(*)cc1] | 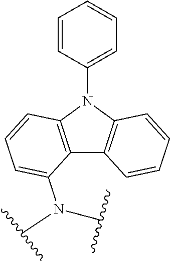 [SMILES: *N(*)c1cccc2c1c1ccccc1n2-c1ccccc1] | 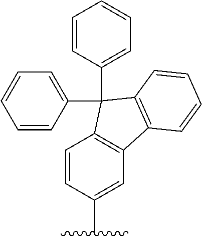 [SMILES: *c1ccc2c(c1)-c1ccccc1C2(c1ccccc1)c1ccccc1] |
| B25 | 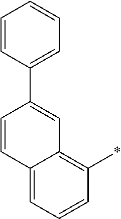 [SMILES: *c1cccc2ccc(-c3ccccc3)cc12] | 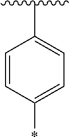 [SMILES: *c1ccc(*)cc1] | 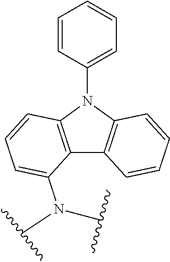 [SMILES: *N(*)c1cccc2c1c1ccccc1n2-c1ccccc1] | 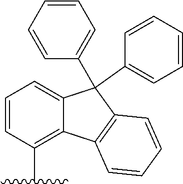 [SMILES: *c1cccc2c1-c1ccccc1C2(c1ccccc1)c1ccccc1] |
| B26 | 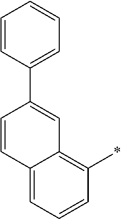 [SMILES: *c1cccc2ccc(-c3ccccc3)cc12] | 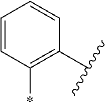 [SMILES: *c1ccccc1*] | 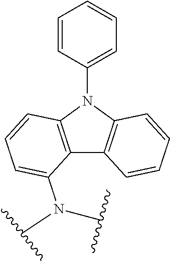 [SMILES: *N(*)c1cccc2c1c1ccccc1n2-c1ccccc1] | 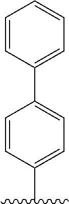 [SMILES: *c1ccc(-c2ccccc2)cc1] |
| B27 | 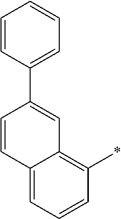 [SMILES: *c1cccc2ccc(-c3ccccc3)cc12] | 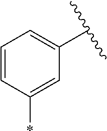 [SMILES: *c1cccc(*)c1] | 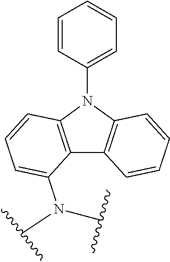 [SMILES: *N(*)c1cccc2c1c1ccccc1n2-c1ccccc1] | 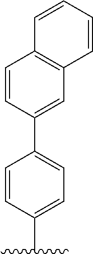 [SMILES: *c1ccc(-c2ccc3ccccc3c2)cc1] |
| B28 | 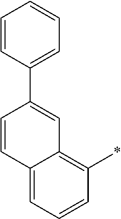 [SMILES: *c1cccc2ccc(-c3ccccc3)cc12] | 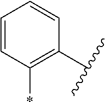 [SMILES: *c1ccccc1*] | 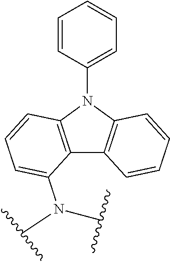 [SMILES: *N(*)c1cccc2c1c1ccccc1n2-c1ccccc1] | 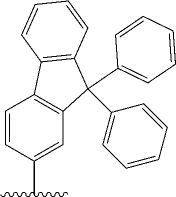 [SMILES: *c1ccc2c(c1)C(c1ccccc1)(c1ccccc1)c1ccccc1-2] |
| B29 | 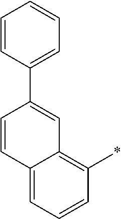 [SMILES: *c1cccc2ccc(-c3ccccc3)cc12] | 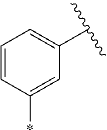 [SMILES: *c1cccc(*)c1] | 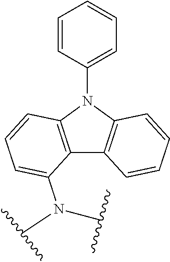 [SMILES: *N(*)c1cccc2c1c1ccccc1n2-c1ccccc1] | 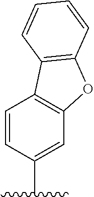 [SMILES: *c1ccc2c(c1)oc1ccccc12] |
| B30 | 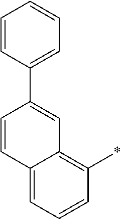 [SMILES: *c1cccc2ccc(-c3ccccc3)cc12] | 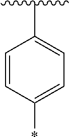 [SMILES: *c1ccc(*)cc1] | 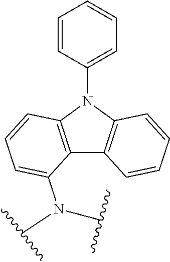 [SMILES: *N(*)c1cccc2c1c1ccccc1n2-c1ccccc1] | 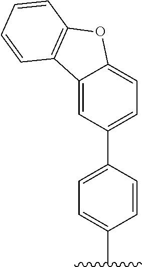 [SMILES: *c1ccc(-c2ccc3oc4ccccc4c3c2)cc1] |
| B31 | 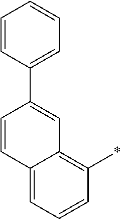 [SMILES: *c1cccc2ccc(-c3ccccc3)cc12] | 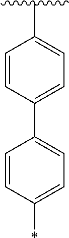 [SMILES: *c1ccc(-c2ccc(*)cc2)cc1] | 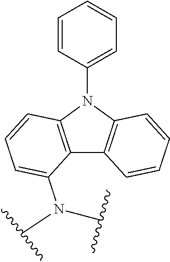 [SMILES: *N(*)c1cccc2c1c1ccccc1n2-c1ccccc1] | 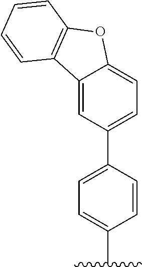 [SMILES: *c1ccc(-c2ccc3oc4ccccc4c3c2)cc1] |
| B32 | 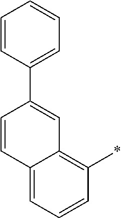 [SMILES: *c1cccc2ccc(-c3ccccc3)cc12] | 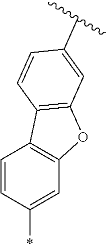 [SMILES: *c1ccc2c(c1)oc1cc(*)ccc12] | 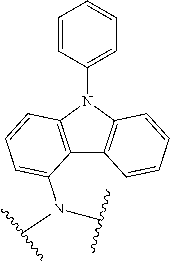 [SMILES: *N(*)c1cccc2c1c1ccccc1n2-c1ccccc1] | 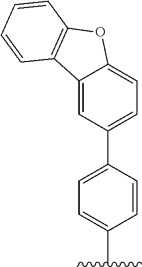 [SMILES: *c1ccc(-c2ccc3oc4ccccc4c3c2)cc1] |
| B33 | 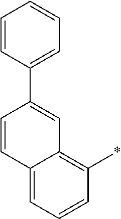 [SMILES: *c1cccc2ccc(-c3ccccc3)cc12] | 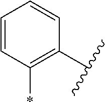 [SMILES: *c1ccccc1*] | 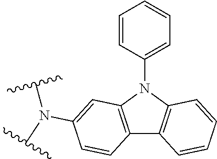 [SMILES: *N(*)c1ccc2c3ccccc3n(-c3ccccc3)c2c1] | 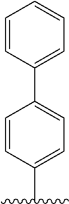 [SMILES: *c1ccc(-c2ccccc2)cc1] |
| B34 | 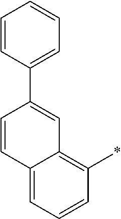 [SMILES: *c1cccc2ccc(-c3ccccc3)cc12] | 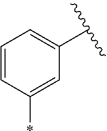 [SMILES: *c1cccc(*)c1] | 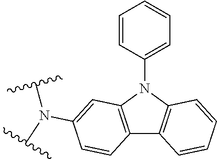 [SMILES: *N(*)c1ccc2c3ccccc3n(-c3ccccc3)c2c1] | 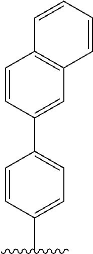 [SMILES: *c1ccc(-c2ccc3ccccc3c2)cc1] |
| B35 | 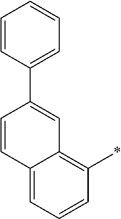 [SMILES: *c1cccc2ccc(-c3ccccc3)cc12] | 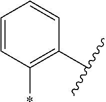 [SMILES: *c1ccccc1*] | 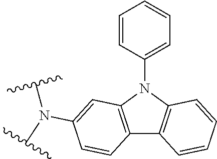 [SMILES: *N(*)c1ccc2c3ccccc3n(-c3ccccc3)c2c1] | 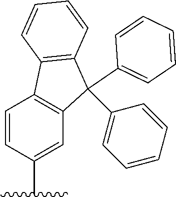 [SMILES: *c1ccc2c(c1)C(c1ccccc1)(c1ccccc1)c1ccccc1-2] |
| B36 | 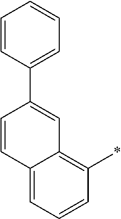 [SMILES: *c1cccc2ccc(-c3ccccc3)cc12] | 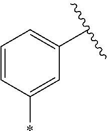 [SMILES: *c1cccc(*)c1] | 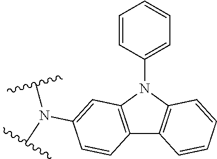 [SMILES: *N(*)c1ccc2c3ccccc3n(-c3ccccc3)c2c1] | 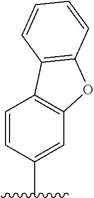 [SMILES: *c1ccc2c(c1)oc1ccccc12] |
| B37 | 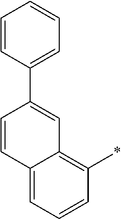 [SMILES: *c1cccc2ccc(-c3ccccc3)cc12] | 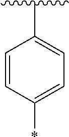 [SMILES: *c1ccc(*)cc1] | 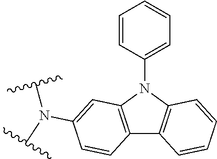 [SMILES: *N(*)c1ccc2c3ccccc3n(-c3ccccc3)c2c1] | 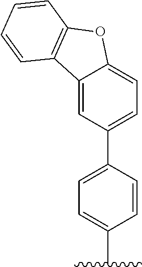 [SMILES: *c1ccc(-c2ccc3oc4ccccc4c3c2)cc1] |
| B38 | 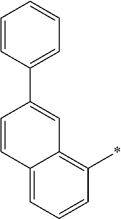 [SMILES: *c1cccc2ccc(-c3ccccc3)cc12] | 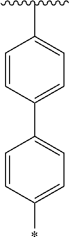 [SMILES: *c1ccc(-c2ccc(*)cc2)cc1] | 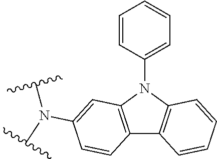 [SMILES: *N(*)c1ccc2c3ccccc3n(-c3ccccc3)c2c1] | 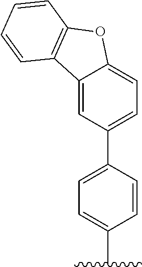 [SMILES: *c1ccc(-c2ccc3oc4ccccc4c3c2)cc1] |
| B39 | 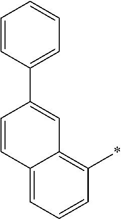 [SMILES: *c1cccc2ccc(-c3ccccc3)cc12] | 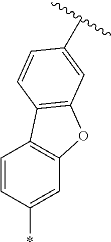 [SMILES: *c1ccc2c(c1)oc1cc(*)ccc12] | 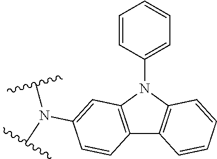 [SMILES: *N(*)c1ccc2c3ccccc3n(-c3ccccc3)c2c1] | 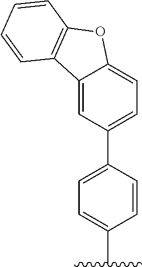 [SMILES: *c1ccc(-c2ccc3oc4ccccc4c3c2)cc1] |
| C1 | 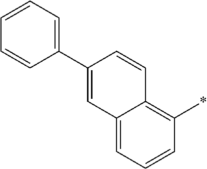 [SMILES: *c1cccc2cc(-c3ccccc3)ccc12] | 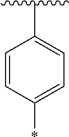 [SMILES: *c1ccc(*)cc1] | 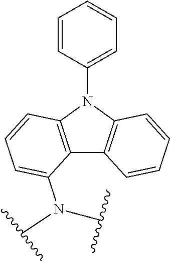 [SMILES: *N(*)c1cccc2c1c1ccccc1n2-c1ccccc1] | 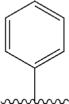 [SMILES: *c1ccccc1] |
| C2 | 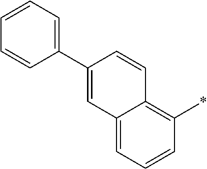 [SMILES: *c1cccc2cc(-c3ccccc3)ccc12] | 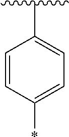 [SMILES: *c1ccc(*)cc1] | 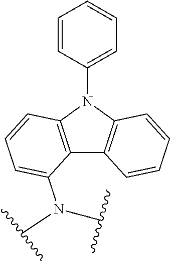 [SMILES: *N(*)c1cccc2c1c1ccccc1n2-c1ccccc1] | 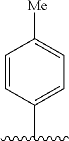 [SMILES: *c1ccc(C)cc1] |
| C3 | 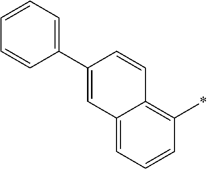 [SMILES: *c1cccc2cc(-c3ccccc3)ccc12] | 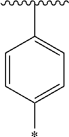 [SMILES: *c1ccc(*)cc1] | 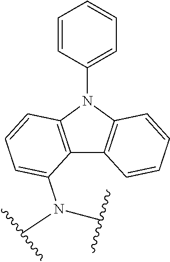 [SMILES: *N(*)c1cccc2c1c1ccccc1n2-c1ccccc1] | 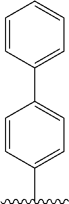 [SMILES: *c1ccc(-c2ccccc2)cc1] |
| C4 | 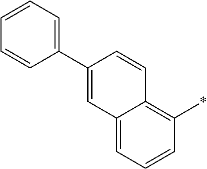 [SMILES: *c1cccc2cc(-c3ccccc3)ccc12] | 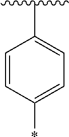 [SMILES: *c1ccc(*)cc1] | 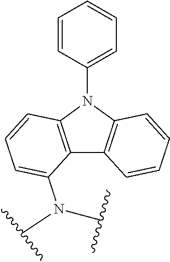 [SMILES: *N(*)c1cccc2c1c1ccccc1n2-c1ccccc1] | 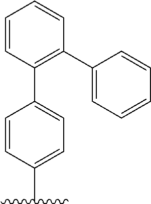 [SMILES: *c1ccc(-c2ccccc2-c2ccccc2)cc1] |
| C5 | 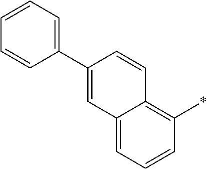 [SMILES: *c1cccc2cc(-c3ccccc3)ccc12] | 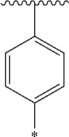 [SMILES: *c1ccc(*)cc1] | 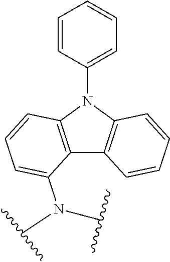 [SMILES: *N(*)c1cccc2c1c1ccccc1n2-c1ccccc1] | 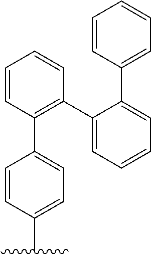 [SMILES: *c1ccc(-c2ccccc2-c2ccccc2-c2ccccc2)cc1] |
| C6 | 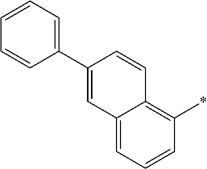 [SMILES: *c1cccc2cc(-c3ccccc3)ccc12] | 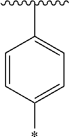 [SMILES: *c1ccc(*)cc1] | 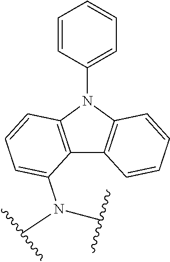 [SMILES: *N(*)c1cccc2c1c1ccccc1n2-c1ccccc1] | 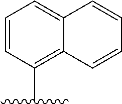 [SMILES: *c1cccc2ccccc12] |
| C7 | 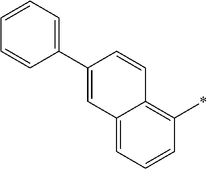 [SMILES: *c1cccc2cc(-c3ccccc3)ccc12] | 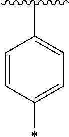 [SMILES: *c1ccc(*)cc1] | 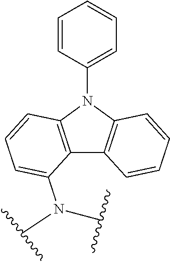 [SMILES: *N(*)c1cccc2c1c1ccccc1n2-c1ccccc1] | 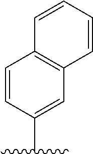 [SMILES: *c1ccc2ccccc2c1] |
| C8 | 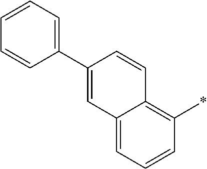 [SMILES: *c1cccc2cc(-c3ccccc3)ccc12] | 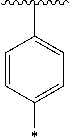 [SMILES: *c1ccc(*)cc1] | 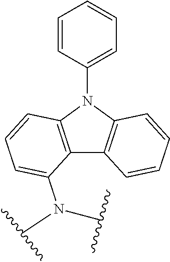 [SMILES: *N(*)c1cccc2c1c1ccccc1n2-c1ccccc1] | 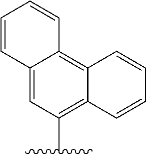 [SMILES: *c1cc2ccccc2c2ccccc12] |
| C9 | 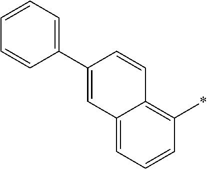 [SMILES: *c1cccc2cc(-c3ccccc3)ccc12] | 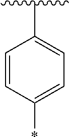 [SMILES: *c1ccc(*)cc1] | 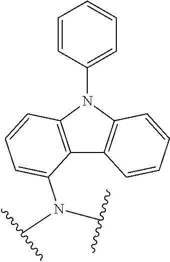 [SMILES: *N(*)c1cccc2c1c1ccccc1n2-c1ccccc1] | 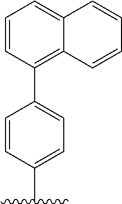 [SMILES: *c1ccc(-c2cccc3ccccc23)cc1] |
| C10 | 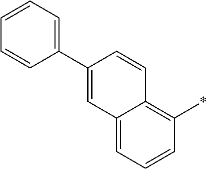 [SMILES: *c1cccc2cc(-c3ccccc3)ccc12] | 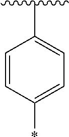 [SMILES: *c1ccc(*)cc1] | 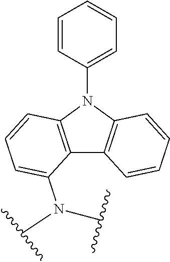 [SMILES: *N(*)c1cccc2c1c1ccccc1n2-c1ccccc1] | 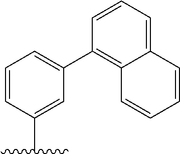 [SMILES: *c1cccc(-c2cccc3ccccc23)c1] |
| C11 | 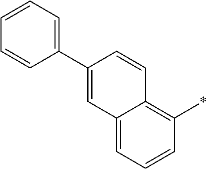 [SMILES: *c1cccc2cc(-c3ccccc3)ccc12] | 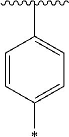 [SMILES: *c1ccc(*)cc1] | 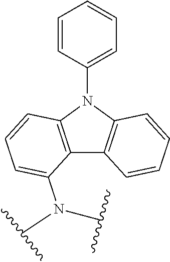 [SMILES: *N(*)c1cccc2c1c1ccccc1n2-c1ccccc1] | 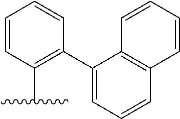 [SMILES: *c1ccccc1-c1cccc2ccccc12] |
| C12 | 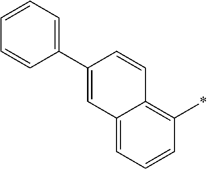 [SMILES: *c1cccc2cc(-c3ccccc3)ccc12] | 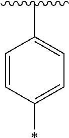 [SMILES: *c1ccc(*)cc1] | 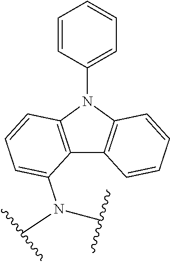 [SMILES: *N(*)c1cccc2c1c1ccccc1n2-c1ccccc1] | 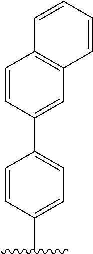 [SMILES: *c1ccc(-c2ccc3ccccc3c2)cc1] |
| C13 | 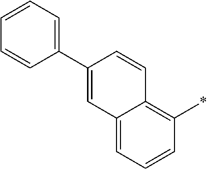 [SMILES: *c1cccc2cc(-c3ccccc3)ccc12] | 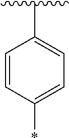 [SMILES: *c1ccc(*)cc1] | 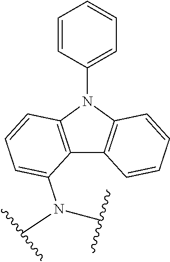 [SMILES: *N(*)c1cccc2c1c1ccccc1n2-c1ccccc1] | 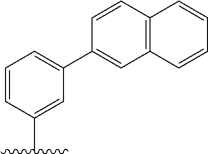 [SMILES: *c1cccc(-c2ccc3ccccc3c2)c1] |
| C14 | 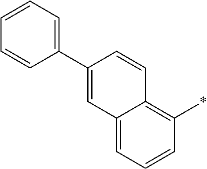 [SMILES: *c1cccc2cc(-c3ccccc3)ccc12] | 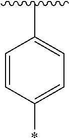 [SMILES: *c1ccc(*)cc1] | 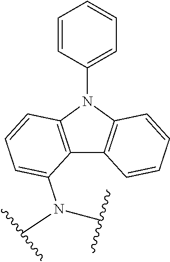 [SMILES: *N(*)c1cccc2c1c1ccccc1n2-c1ccccc1] | 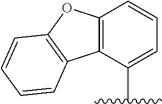 [SMILES: *c1cccc2oc3ccccc3c12] |
| C15 | 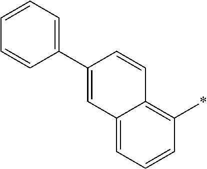 [SMILES: *c1cccc2cc(-c3ccccc3)ccc12] | 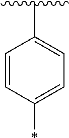 [SMILES: *c1ccc(*)cc1] | 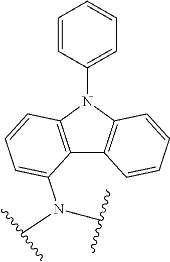 [SMILES: *N(*)c1cccc2c1c1ccccc1n2-c1ccccc1] | 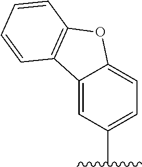 [SMILES: *c1ccc2oc3ccccc3c2c1] |
| C16 | 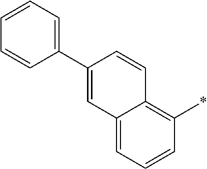 [SMILES: *c1cccc2cc(-c3ccccc3)ccc12] | 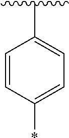 [SMILES: *c1ccc(*)cc1] | 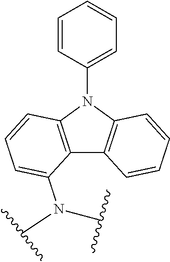 [SMILES: *N(*)c1cccc2c1c1ccccc1n2-c1ccccc1] | 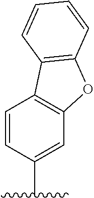 [SMILES: *c1ccc2c(c1)oc1ccccc12] |
| C17 | 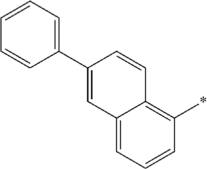 [SMILES: *c1cccc2cc(-c3ccccc3)ccc12] | 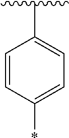 [SMILES: *c1ccc(*)cc1] | 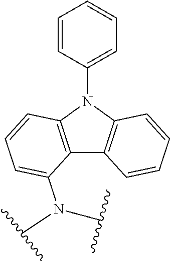 [SMILES: *N(*)c1cccc2c1c1ccccc1n2-c1ccccc1] | 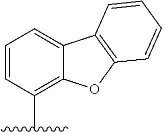 [SMILES: *c1cccc2c1oc1ccccc12] |
| C18 | 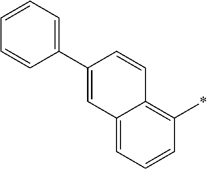 [SMILES: *c1cccc2cc(-c3ccccc3)ccc12] | 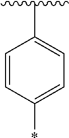 [SMILES: *c1ccc(*)cc1] | 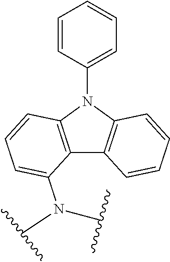 [SMILES: *N(*)c1cccc2c1c1ccccc1n2-c1ccccc1] | 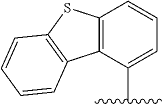 [SMILES: *c1cccc2sc3ccccc3c12] |
| C19 | 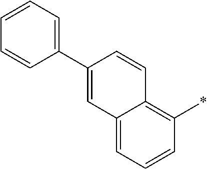 [SMILES: *c1cccc2cc(-c3ccccc3)ccc12] | 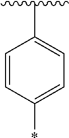 [SMILES: *c1ccc(*)cc1] | 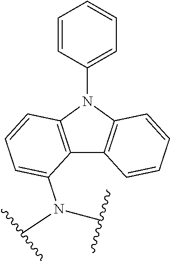 [SMILES: *N(*)c1cccc2c1c1ccccc1n2-c1ccccc1] | 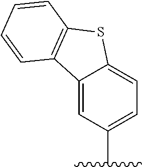 [SMILES: *c1ccc2sc3ccccc3c2c1] |
| C20 | 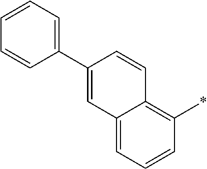 [SMILES: *c1cccc2cc(-c3ccccc3)ccc12] | 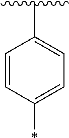 [SMILES: *c1ccc(*)cc1] | 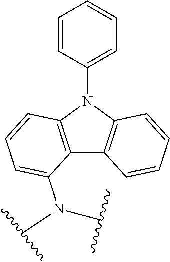 [SMILES: *N(*)c1cccc2c1c1ccccc1n2-c1ccccc1] | 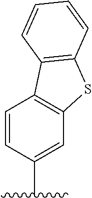 [SMILES: *c1ccc2c(c1)sc1ccccc12] |
| C21 | 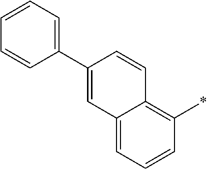 [SMILES: *c1cccc2cc(-c3ccccc3)ccc12] | 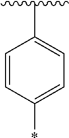 [SMILES: *c1ccc(*)cc1] | 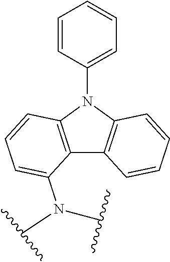 [SMILES: *N(*)c1cccc2c1c1ccccc1n2-c1ccccc1] | 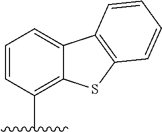 [SMILES: *c1cccc2c1sc1ccccc12] |
| C22 | 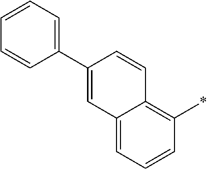 [SMILES: *c1cccc2cc(-c3ccccc3)ccc12] | 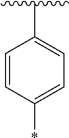 [SMILES: *c1ccc(*)cc1] | 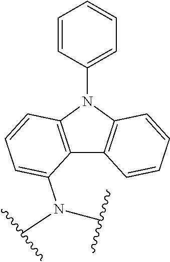 [SMILES: *N(*)c1cccc2c1c1ccccc1n2-c1ccccc1] | 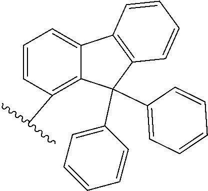 [SMILES: *c1cccc2c1C(c1ccccc1)(c1ccccc1)c1ccccc1-2] |
| C23 | 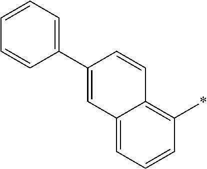 [SMILES: *c1cccc2cc(-c3ccccc3)ccc12] | 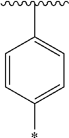 [SMILES: *c1ccc(*)cc1] | 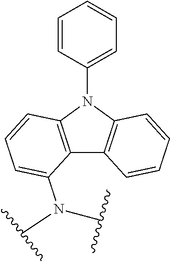 [SMILES: *N(*)c1cccc2c1c1ccccc1n2-c1ccccc1] | 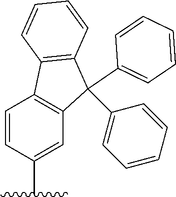 [SMILES: *c1ccc2c(c1)C(c1ccccc1)(c1ccccc1)c1ccccc1-2] |
| C24 | 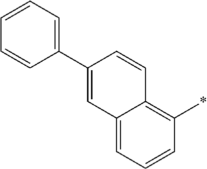 [SMILES: *c1cccc2cc(-c3ccccc3)ccc12] | 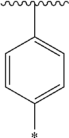 [SMILES: *c1ccc(*)cc1] | 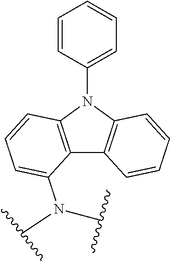 [SMILES: *N(*)c1cccc2c1c1ccccc1n2-c1ccccc1] | 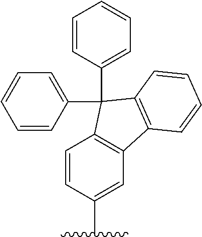 [SMILES: *c1ccc2c(c1)-c1ccccc1C2(c1ccccc1)c1ccccc1] |
| C25 | 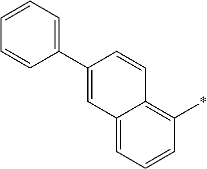 [SMILES: *c1cccc2cc(-c3ccccc3)ccc12] | 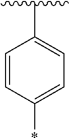 [SMILES: *c1ccc(*)cc1] | 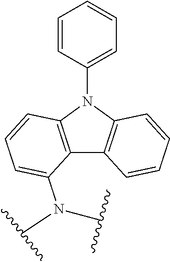 [SMILES: *N(*)c1cccc2c1c1ccccc1n2-c1ccccc1] | 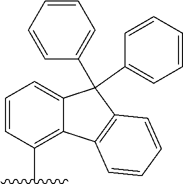 [SMILES: *c1cccc2c1-c1ccccc1C2(c1ccccc1)c1ccccc1] |
| C26 | 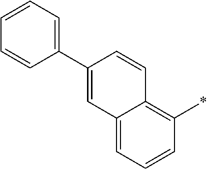 [SMILES: *c1cccc2cc(-c3ccccc3)ccc12] | 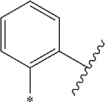 [SMILES: *c1ccccc1*] | 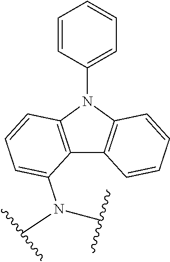 [SMILES: *N(*)c1cccc2c1c1ccccc1n2-c1ccccc1] | 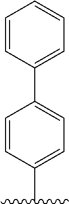 [SMILES: *c1ccc(-c2ccccc2)cc1] |
| C27 | 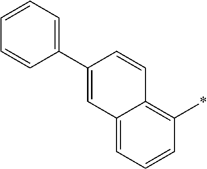 [SMILES: *c1cccc2cc(-c3ccccc3)ccc12] | 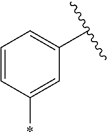 [SMILES: *c1cccc(*)c1] | 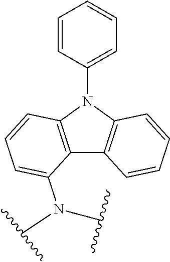 [SMILES: *N(*)c1cccc2c1c1ccccc1n2-c1ccccc1] | 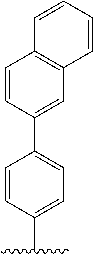 [SMILES: *c1ccc(-c2ccc3ccccc3c2)cc1] |
| C28 | 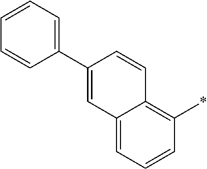 [SMILES: *c1cccc2cc(-c3ccccc3)ccc12] | 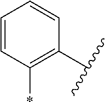 [SMILES: *c1ccccc1*] | 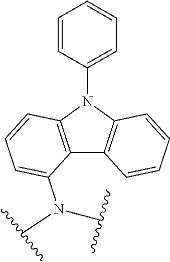 [SMILES: *N(*)c1cccc2c1c1ccccc1n2-c1ccccc1] | 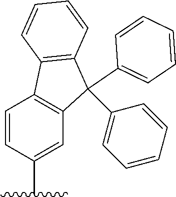 [SMILES: *c1ccc2c(c1)C(c1ccccc1)(c1ccccc1)c1ccccc1-2] |
| C29 | 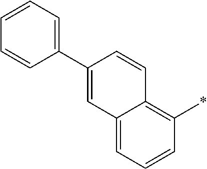 [SMILES: *c1cccc2cc(-c3ccccc3)ccc12] | 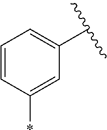 [SMILES: *c1cccc(*)c1] | 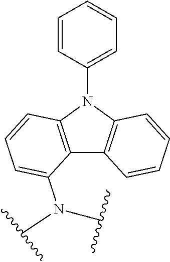 [SMILES: *N(*)c1cccc2c1c1ccccc1n2-c1ccccc1] | 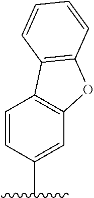 [SMILES: *c1ccc2c(c1)oc1ccccc12] |
| C30 | 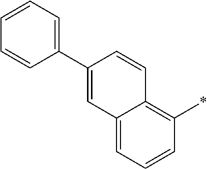 [SMILES: *c1cccc2cc(-c3ccccc3)ccc12] | 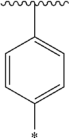 [SMILES: *c1ccc(*)cc1] | 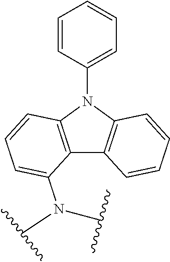 [SMILES: *N(*)c1cccc2c1c1ccccc1n2-c1ccccc1] | 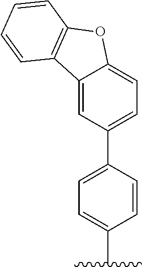 [SMILES: *c1ccc(-c2ccc3oc4ccccc4c3c2)cc1] |
| C31 | 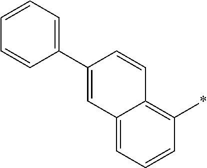 [SMILES: *c1cccc2cc(-c3ccccc3)ccc12] | 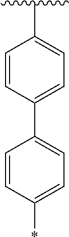 [SMILES: *c1ccc(-c2ccc(*)cc2)cc1] | 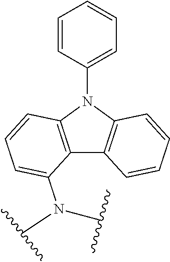 [SMILES: *N(*)c1cccc2c1c1ccccc1n2-c1ccccc1] | 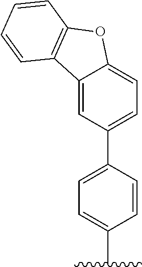 [SMILES: *c1ccc(-c2ccc3oc4ccccc4c3c2)cc1] |
| C32 | 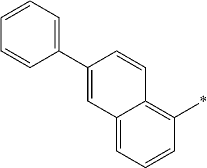 [SMILES: *c1cccc2cc(-c3ccccc3)ccc12] | 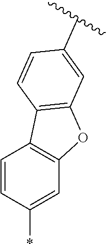 [SMILES: *c1ccc2c(c1)oc1cc(*)ccc12] | 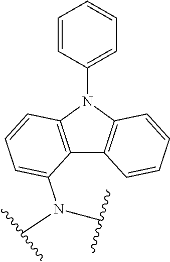 [SMILES: *N(*)c1cccc2c1c1ccccc1n2-c1ccccc1] | 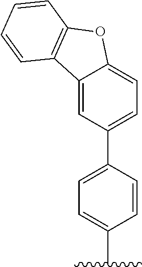 [SMILES: *c1ccc(-c2ccc3oc4ccccc4c3c2)cc1] |
| C33 | 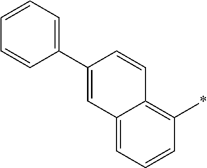 [SMILES: *c1cccc2cc(-c3ccccc3)ccc12] | 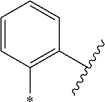 [SMILES: *c1ccccc1*] | 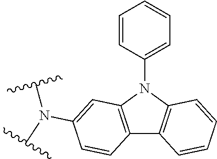 [SMILES: *N(*)c1ccc2c3ccccc3n(-c3ccccc3)c2c1] | 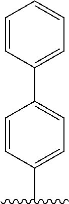 [SMILES: *c1ccc(-c2ccccc2)cc1] |
| C34 | 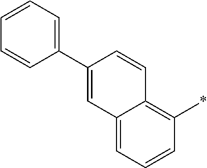 [SMILES: *c1cccc2cc(-c3ccccc3)ccc12] | 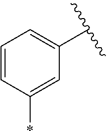 [SMILES: *c1cccc(*)c1] | 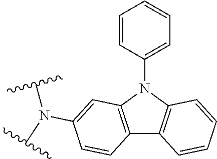 [SMILES: *N(*)c1ccc2c3ccccc3n(-c3ccccc3)c2c1] | 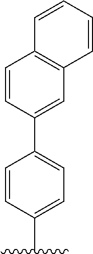 [SMILES: *c1ccc(-c2ccc3ccccc3c2)cc1] |
| C35 | 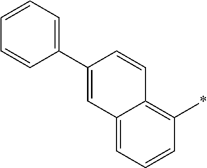 [SMILES: *c1cccc2cc(-c3ccccc3)ccc12] | 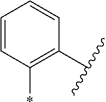 [SMILES: *c1ccccc1*] | 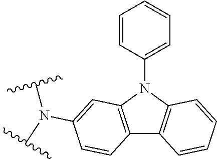 [SMILES: *N(*)c1ccc2c3ccccc3n(-c3ccccc3)c2c1] | 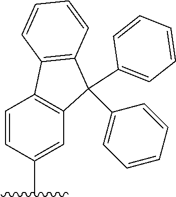 [SMILES: *c1ccc2c(c1)C(c1ccccc1)(c1ccccc1)c1ccccc1-2] |
| C36 | 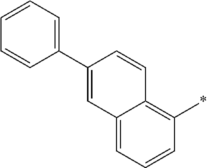 [SMILES: *c1cccc2cc(-c3ccccc3)ccc12] | 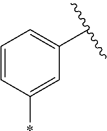 [SMILES: *c1cccc(*)c1] | 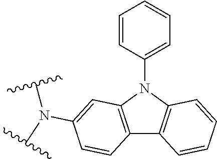 [SMILES: *N(*)c1ccc2c3ccccc3n(-c3ccccc3)c2c1] | 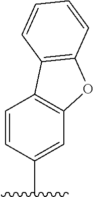 [SMILES: *c1ccc2c(c1)oc1ccccc12] |
| C37 | 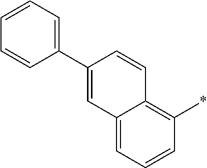 [SMILES: *c1cccc2cc(-c3ccccc3)ccc12] | 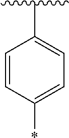 [SMILES: *c1ccc(*)cc1] | 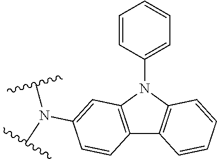 [SMILES: *N(*)c1ccc2c3ccccc3n(-c3ccccc3)c2c1] | 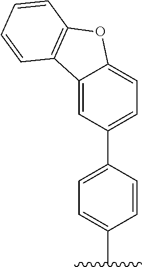 [SMILES: *c1ccc(-c2ccc3oc4ccccc4c3c2)cc1] |
| C38 | 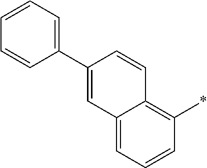 [SMILES: *c1cccc2cc(-c3ccccc3)ccc12] | 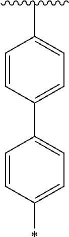 [SMILES: *c1ccc(-c2ccc(*)cc2)cc1] | 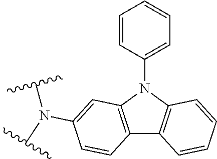 [SMILES: *N(*)c1ccc2c3ccccc3n(-c3ccccc3)c2c1] | 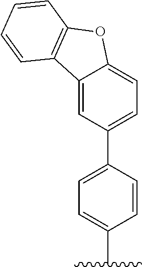 [SMILES: *c1ccc(-c2ccc3oc4ccccc4c3c2)cc1] |
| C39 | 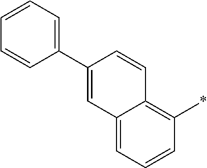 [SMILES: *c1cccc2cc(-c3ccccc3)ccc12] | 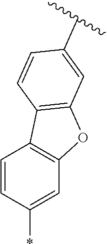 [SMILES: *c1ccc2c(c1)oc1cc(*)ccc12] | 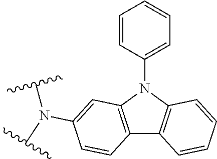 [SMILES: *N(*)c1ccc2c3ccccc3n(-c3ccccc3)c2c1] | 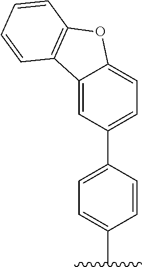 [SMILES: *c1ccc(-c2ccc3oc4ccccc4c3c2)cc1] |
| D1 | 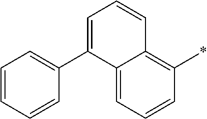 [SMILES: *c1cccc2c(-c3ccccc3)cccc12] | 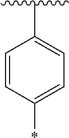 [SMILES: *c1ccc(*)cc1] | 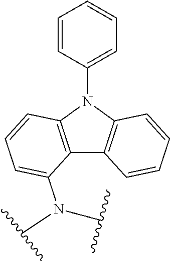 [SMILES: *N(*)c1cccc2c1c1ccccc1n2-c1ccccc1] | 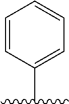 [SMILES: *c1ccccc1] |
| D2 | 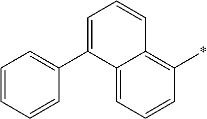 [SMILES: *c1cccc2c(-c3ccccc3)cccc12] | 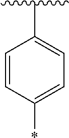 [SMILES: *c1ccc(*)cc1] | 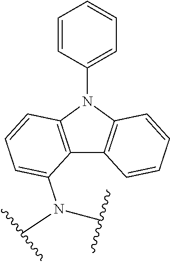 [SMILES: *N(*)c1cccc2c1c1ccccc1n2-c1ccccc1] | 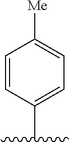 [SMILES: *c1ccc(C)cc1] |
| D3 | 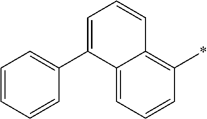 [SMILES: *c1cccc2c(-c3ccccc3)cccc12] | 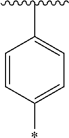 [SMILES: *c1ccc(*)cc1] | 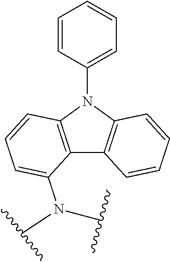 [SMILES: *N(*)c1cccc2c1c1ccccc1n2-c1ccccc1] | 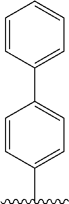 [SMILES: *c1ccc(-c2ccccc2)cc1] |
| D4 | 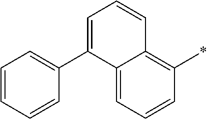 [SMILES: *c1cccc2c(-c3ccccc3)cccc12] | 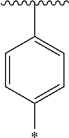 [SMILES: *c1ccc(*)cc1] | 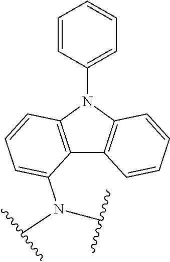 [SMILES: *N(*)c1cccc2c1c1ccccc1n2-c1ccccc1] | 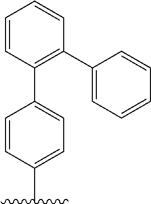 [SMILES: *c1ccc(-c2ccccc2-c2ccccc2)cc1] |
| D5 | 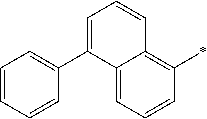 [SMILES: *c1cccc2c(-c3ccccc3)cccc12] | 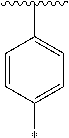 [SMILES: *c1ccc(*)cc1] | 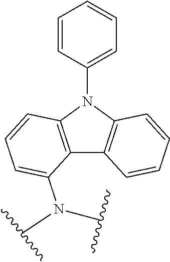 [SMILES: *N(*)c1cccc2c1c1ccccc1n2-c1ccccc1] | 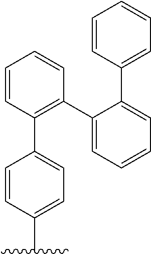 [SMILES: *c1ccc(-c2ccccc2-c2ccccc2-c2ccccc2)cc1] |
| D6 | 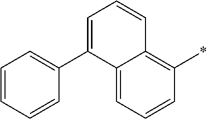 [SMILES: *c1cccc2c(-c3ccccc3)cccc12] | 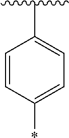 [SMILES: *c1ccc(*)cc1] | 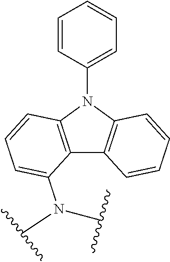 [SMILES: *N(*)c1cccc2c1c1ccccc1n2-c1ccccc1] | 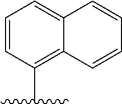 [SMILES: *c1cccc2ccccc12] |
| D7 | 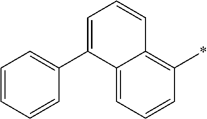 [SMILES: *c1cccc2c(-c3ccccc3)cccc12] | 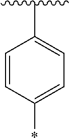 [SMILES: *c1ccc(*)cc1] | 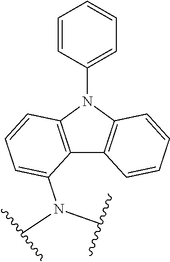 [SMILES: *N(*)c1cccc2c1c1ccccc1n2-c1ccccc1] | 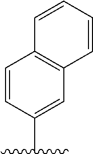 [SMILES: *c1ccc2ccccc2c1] |
| D8 | 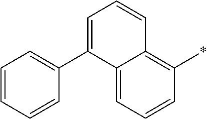 [SMILES: *c1cccc2c(-c3ccccc3)cccc12] | 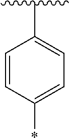 [SMILES: *c1ccc(*)cc1] | 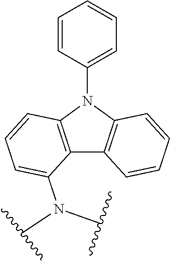 [SMILES: *N(*)c1cccc2c1c1ccccc1n2-c1ccccc1] | 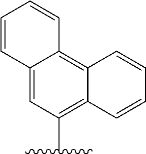 [SMILES: *c1cc2ccccc2c2ccccc12] |
| D9 | 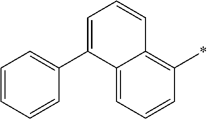 [SMILES: *c1cccc2c(-c3ccccc3)cccc12] | 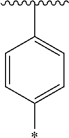 [SMILES: *c1ccc(*)cc1] | 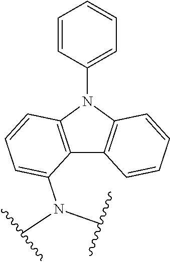 [SMILES: *N(*)c1cccc2c1c1ccccc1n2-c1ccccc1] | 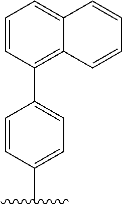 [SMILES: *c1ccc(-c2cccc3ccccc23)cc1] |
| D10 | 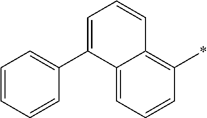 [SMILES: *c1cccc2c(-c3ccccc3)cccc12] | 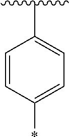 [SMILES: *c1ccc(*)cc1] | 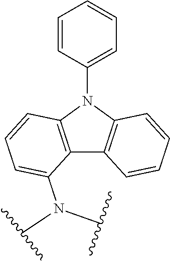 [SMILES: *N(*)c1cccc2c1c1ccccc1n2-c1ccccc1] | 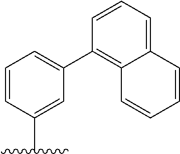 [SMILES: *c1cccc(-c2cccc3ccccc23)c1] |
| D11 | 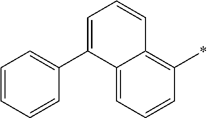 [SMILES: *c1cccc2c(-c3ccccc3)cccc12] | 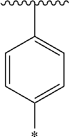 [SMILES: *c1ccc(*)cc1] | 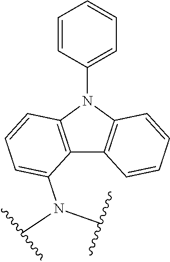 [SMILES: *N(*)c1cccc2c1c1ccccc1n2-c1ccccc1] | 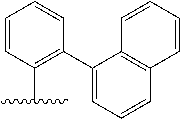 [SMILES: *c1ccccc1-c1cccc2ccccc12] |
| D12 | 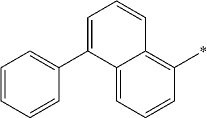 [SMILES: *c1cccc2c(-c3ccccc3)cccc12] | 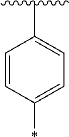 [SMILES: *c1ccc(*)cc1] | 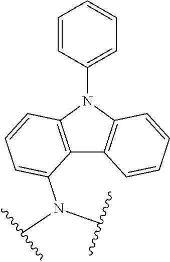 [SMILES: *N(*)c1cccc2c1c1ccccc1n2-c1ccccc1] | 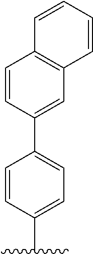 [SMILES: *c1ccc(-c2ccc3ccccc3c2)cc1] |
| D13 | 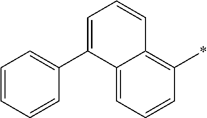 [SMILES: *c1cccc2c(-c3ccccc3)cccc12] | 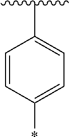 [SMILES: *c1ccc(*)cc1] | 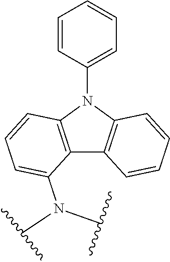 [SMILES: *N(*)c1cccc2c1c1ccccc1n2-c1ccccc1] | 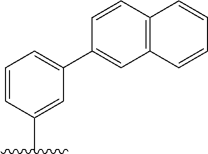 [SMILES: *c1cccc(-c2ccc3ccccc3c2)c1] |
| D14 | 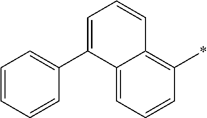 [SMILES: *c1cccc2c(-c3ccccc3)cccc12] | 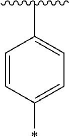 [SMILES: *c1ccc(*)cc1] | 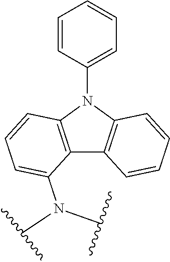 [SMILES: *N(*)c1cccc2c1c1ccccc1n2-c1ccccc1] | 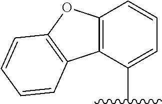 [SMILES: *c1cccc2oc3ccccc3c12] |
| D15 | 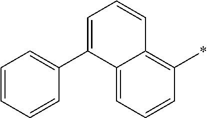 [SMILES: *c1cccc2c(-c3ccccc3)cccc12] | 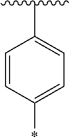 [SMILES: *c1ccc(*)cc1] | 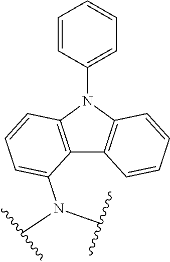 [SMILES: *N(*)c1cccc2c1c1ccccc1n2-c1ccccc1] | 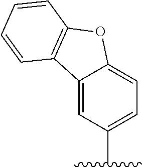 [SMILES: *c1ccc2oc3ccccc3c2c1] |
| D16 | 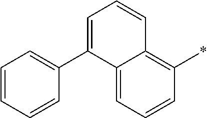 [SMILES: *c1cccc2c(-c3ccccc3)cccc12] | 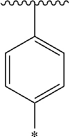 [SMILES: *c1ccc(*)cc1] | 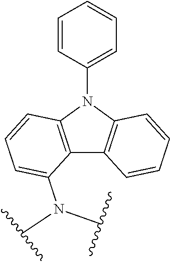 [SMILES: *N(*)c1cccc2c1c1ccccc1n2-c1ccccc1] | 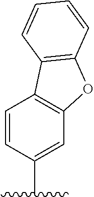 [SMILES: *c1ccc2c(c1)oc1ccccc12] |
| D17 | 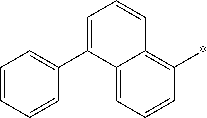 [SMILES: *c1cccc2c(-c3ccccc3)cccc12] | 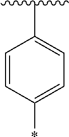 [SMILES: *c1ccc(*)cc1] | 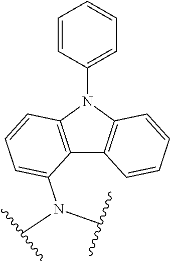 [SMILES: *N(*)c1cccc2c1c1ccccc1n2-c1ccccc1] | 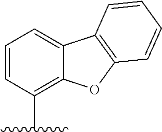 [SMILES: *c1cccc2c1oc1ccccc12] |
| D18 | 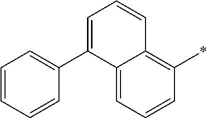 [SMILES: *c1cccc2c(-c3ccccc3)cccc12] | 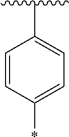 [SMILES: *c1ccc(*)cc1] | 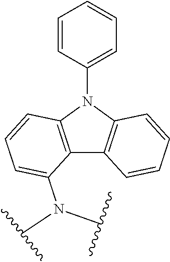 [SMILES: *N(*)c1cccc2c1c1ccccc1n2-c1ccccc1] | 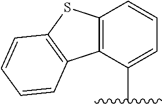 [SMILES: *c1cccc2sc3ccccc3c12] |
| D19 | 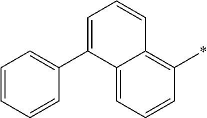 [SMILES: *c1cccc2c(-c3ccccc3)cccc12] | 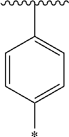 [SMILES: *c1ccc(*)cc1] | 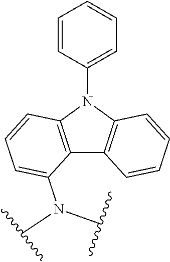 [SMILES: *N(*)c1cccc2c1c1ccccc1n2-c1ccccc1] | 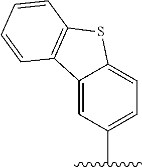 [SMILES: *c1ccc2sc3ccccc3c2c1] |
| D20 | 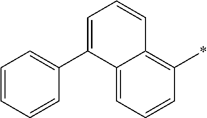 [SMILES: *c1cccc2c(-c3ccccc3)cccc12] | 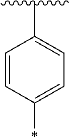 [SMILES: *c1ccc(*)cc1] | 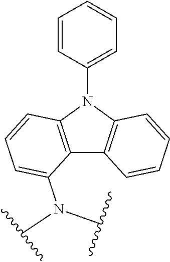 [SMILES: *N(*)c1cccc2c1c1ccccc1n2-c1ccccc1] | 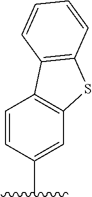 [SMILES: *c1ccc2c(c1)sc1ccccc12] |
| D21 | 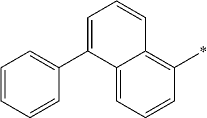 [SMILES: *c1cccc2c(-c3ccccc3)cccc12] | 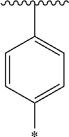 [SMILES: *c1ccc(*)cc1] | 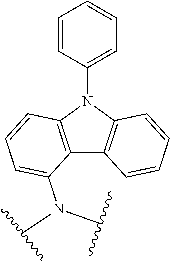 [SMILES: *N(*)c1cccc2c1c1ccccc1n2-c1ccccc1] | 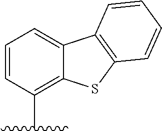 [SMILES: *c1cccc2c1sc1ccccc12] |
| D22 | 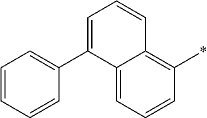 [SMILES: *c1cccc2c(-c3ccccc3)cccc12] | 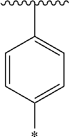 [SMILES: *c1ccc(*)cc1] | 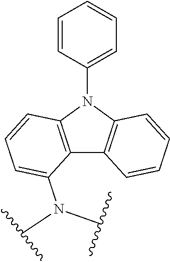 [SMILES: *N(*)c1cccc2c1c1ccccc1n2-c1ccccc1] | 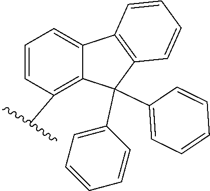 [SMILES: *c1cccc2c1C(c1ccccc1)(c1ccccc1)c1ccccc1-2] |
| D23 | 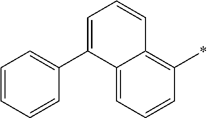 [SMILES: *c1cccc2c(-c3ccccc3)cccc12] | 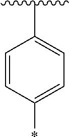 [SMILES: *c1ccc(*)cc1] | 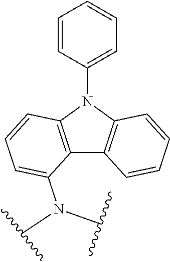 [SMILES: *N(*)c1cccc2c1c1ccccc1n2-c1ccccc1] | 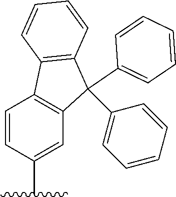 [SMILES: *c1ccc2c(c1)C(c1ccccc1)(c1ccccc1)c1ccccc1-2] |
| D24 | 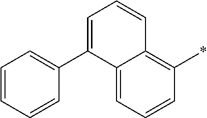 [SMILES: *c1cccc2c(-c3ccccc3)cccc12] | 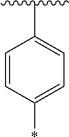 [SMILES: *c1ccc(*)cc1] | 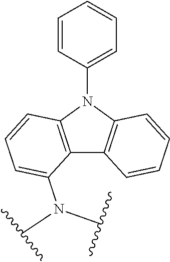 [SMILES: *N(*)c1cccc2c1c1ccccc1n2-c1ccccc1] | 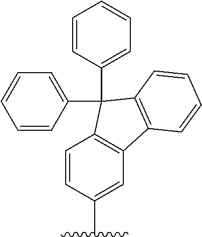 [SMILES: *c1ccc2c(c1)-c1ccccc1C2(c1ccccc1)c1ccccc1] |
| D25 | 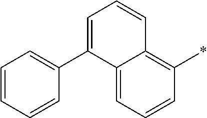 [SMILES: *c1cccc2c(-c3ccccc3)cccc12] | 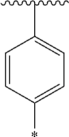 [SMILES: *c1ccc(*)cc1] | 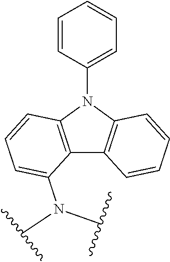 [SMILES: *N(*)c1cccc2c1c1ccccc1n2-c1ccccc1] | 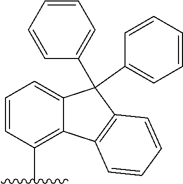 [SMILES: *c1cccc2c1-c1ccccc1C2(c1ccccc1)c1ccccc1] |
| D26 | 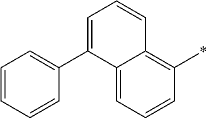 [SMILES: *c1cccc2c(-c3ccccc3)cccc12] | 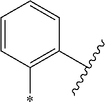 [SMILES: *c1ccccc1*] | 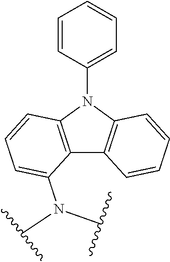 [SMILES: *N(*)c1cccc2c1c1ccccc1n2-c1ccccc1] | 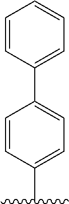 [SMILES: *c1ccc(-c2ccccc2)cc1] |
| D27 | 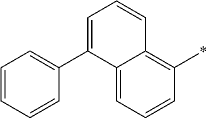 [SMILES: *c1cccc2c(-c3ccccc3)cccc12] | 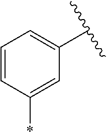 [SMILES: *c1cccc(*)c1] | 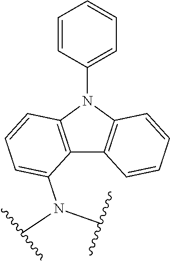 [SMILES: *N(*)c1cccc2c1c1ccccc1n2-c1ccccc1] | 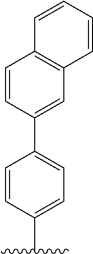 [SMILES: *c1ccc(-c2ccc3ccccc3c2)cc1] |
| D28 | 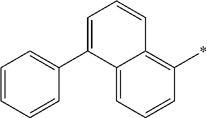 [SMILES: *c1cccc2c(-c3ccccc3)cccc12] | 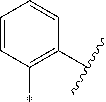 [SMILES: *c1ccccc1*] | 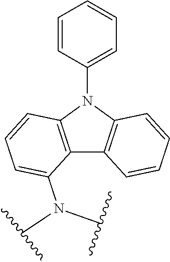 [SMILES: *N(*)c1cccc2c1c1ccccc1n2-c1ccccc1] | 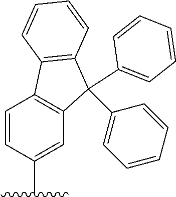 [SMILES: *c1ccc2c(c1)C(c1ccccc1)(c1ccccc1)c1ccccc1-2] |
| D29 | 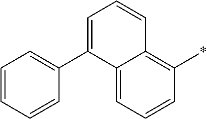 [SMILES: *c1cccc2c(-c3ccccc3)cccc12] | 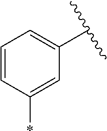 [SMILES: *c1cccc(*)c1] | 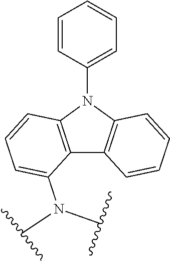 [SMILES: *N(*)c1cccc2c1c1ccccc1n2-c1ccccc1] | 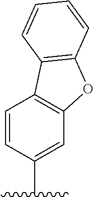 [SMILES: *c1ccc2c(c1)oc1ccccc12] |
| D30 | 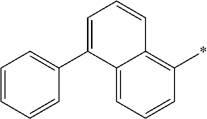 [SMILES: *c1cccc2c(-c3ccccc3)cccc12] | 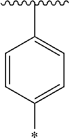 [SMILES: *c1ccc(*)cc1] | 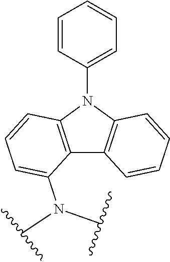 [SMILES: *N(*)c1cccc2c1c1ccccc1n2-c1ccccc1] | 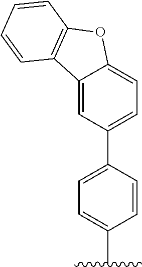 [SMILES: *c1ccc(-c2ccc3oc4ccccc4c3c2)cc1] |
| D31 | 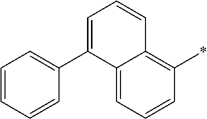 [SMILES: *c1cccc2c(-c3ccccc3)cccc12] | 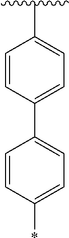 [SMILES: *c1ccc(-c2ccc(*)cc2)cc1] | 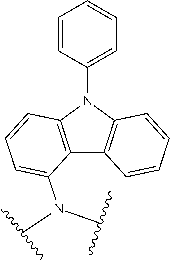 [SMILES: *N(*)c1cccc2c1c1ccccc1n2-c1ccccc1] | 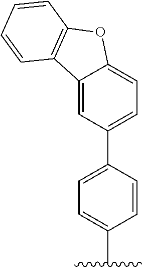 [SMILES: *c1ccc(-c2ccc3oc4ccccc4c3c2)cc1] |
| D32 | 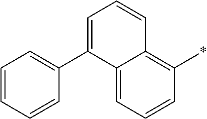 [SMILES: *c1cccc2c(-c3ccccc3)cccc12] | 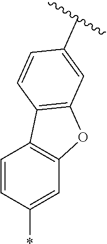 [SMILES: *c1ccc2c(c1)oc1cc(*)ccc12] | 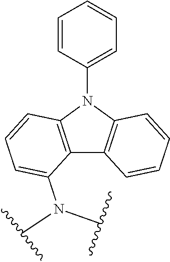 [SMILES: *N(*)c1cccc2c1c1ccccc1n2-c1ccccc1] | 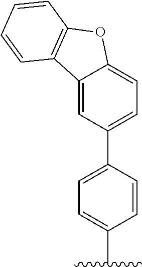 [SMILES: *c1ccc(-c2ccc3oc4ccccc4c3c2)cc1] |
| D33 | 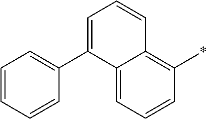 [SMILES: *c1cccc2c(-c3ccccc3)cccc12] | 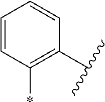 [SMILES: *c1ccccc1*] | 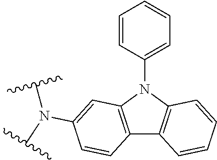 [SMILES: *N(*)c1ccc2c3ccccc3n(-c3ccccc3)c2c1] | 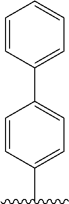 [SMILES: *c1ccc(-c2ccccc2)cc1] |
| D34 | 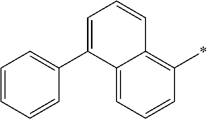 [SMILES: *c1cccc2c(-c3ccccc3)cccc12] | 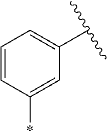 [SMILES: *c1cccc(*)c1] | 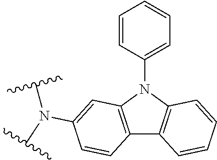 [SMILES: *N(*)c1ccc2c3ccccc3n(-c3ccccc3)c2c1] | 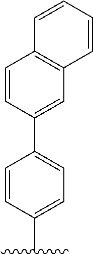 [SMILES: *c1ccc(-c2ccc3ccccc3c2)cc1] |
| D35 | 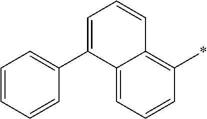 [SMILES: *c1cccc2c(-c3ccccc3)cccc12] | 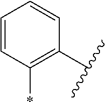 [SMILES: *c1ccccc1*] | 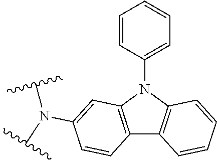 [SMILES: *N(*)c1ccc2c3ccccc3n(-c3ccccc3)c2c1] | 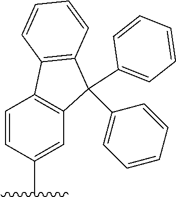 [SMILES: *c1ccc2c(c1)C(c1ccccc1)(c1ccccc1)c1ccccc1-2] |
| D36 | 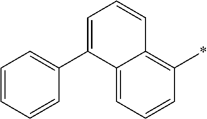 [SMILES: *c1cccc2c(-c3ccccc3)cccc12] | 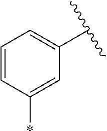 [SMILES: *c1cccc(*)c1] | 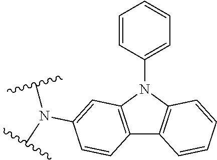 [SMILES: *N(*)c1ccc2c3ccccc3n(-c3ccccc3)c2c1] | 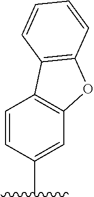 [SMILES: *c1ccc2c(c1)oc1ccccc12] |
| D37 | 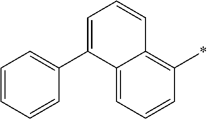 [SMILES: *c1cccc2c(-c3ccccc3)cccc12] | 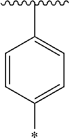 [SMILES: *c1ccc(*)cc1] | 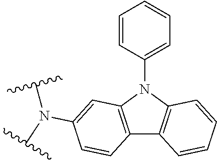 [SMILES: *N(*)c1ccc2c3ccccc3n(-c3ccccc3)c2c1] | 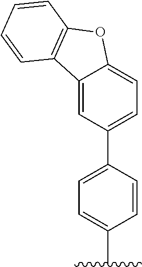 [SMILES: *c1ccc(-c2ccc3oc4ccccc4c3c2)cc1] |
| D38 | 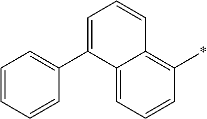 [SMILES: *c1cccc2c(-c3ccccc3)cccc12] | 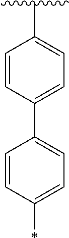 [SMILES: *c1ccc(-c2ccc(*)cc2)cc1] | 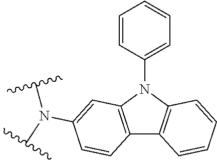 [SMILES: *N(*)c1ccc2c3ccccc3n(-c3ccccc3)c2c1] | 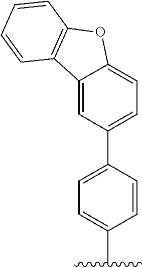 [SMILES: *c1ccc(-c2ccc3oc4ccccc4c3c2)cc1] |
| D39 | 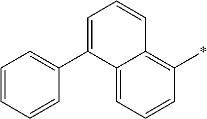 [SMILES: *c1cccc2c(-c3ccccc3)cccc12] | 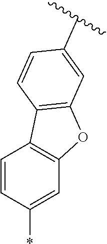 [SMILES: *c1ccc2c(c1)oc1cc(*)ccc12] | 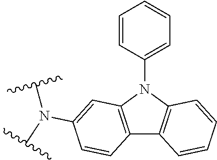 [SMILES: *N(*)c1ccc2c3ccccc3n(-c3ccccc3)c2c1] | 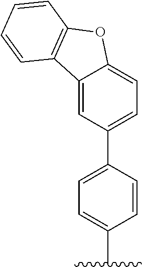 [SMILES: *c1ccc(-c2ccc3oc4ccccc4c3c2)cc1] |
| E1 | 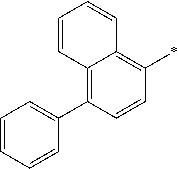 [SMILES: *c1ccc(-c2ccccc2)c2ccccc12] | 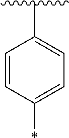 [SMILES: *c1ccc(*)cc1] | 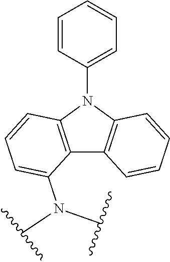 [SMILES: *N(*)c1cccc2c1c1ccccc1n2-c1ccccc1] | 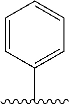 [SMILES: *c1ccccc1] |
| E2 | 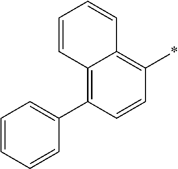 [SMILES: *c1ccc(-c2ccccc2)c2ccccc12] | 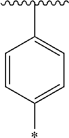 [SMILES: *c1ccc(*)cc1] | 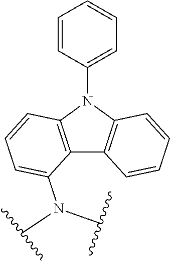 [SMILES: *N(*)c1cccc2c1c1ccccc1n2-c1ccccc1] | 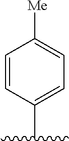 [SMILES: *c1ccc(C)cc1] |
| E3 | 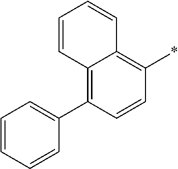 [SMILES: *c1ccc(-c2ccccc2)c2ccccc12] | 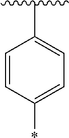 [SMILES: *c1ccc(*)cc1] | 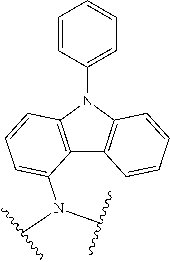 [SMILES: *N(*)c1cccc2c1c1ccccc1n2-c1ccccc1] | 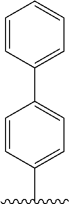 [SMILES: *c1ccc(-c2ccccc2)cc1] |
| E4 | 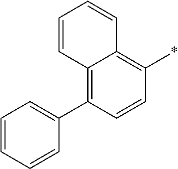 [SMILES: *c1ccc(-c2ccccc2)c2ccccc12] | 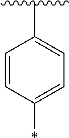 [SMILES: *c1ccc(*)cc1] | 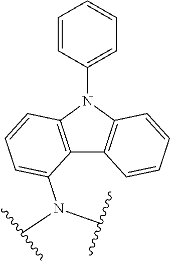 [SMILES: *N(*)c1cccc2c1c1ccccc1n2-c1ccccc1] | 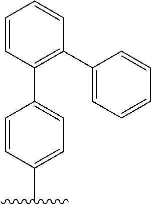 [SMILES: *c1ccc(-c2ccccc2-c2ccccc2)cc1] |
| E5 | 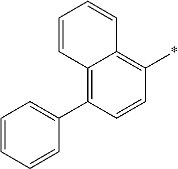 [SMILES: *c1ccc(-c2ccccc2)c2ccccc12] | 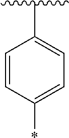 [SMILES: *c1ccc(*)cc1] | 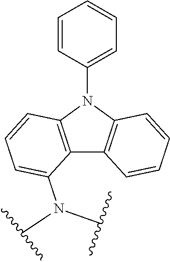 [SMILES: *N(*)c1cccc2c1c1ccccc1n2-c1ccccc1] | 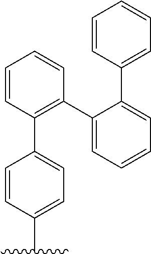 [SMILES: *c1ccc(-c2ccccc2-c2ccccc2-c2ccccc2)cc1] |
| E6 | 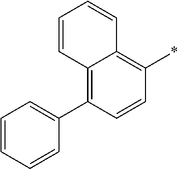 [SMILES: *c1ccc(-c2ccccc2)c2ccccc12] | 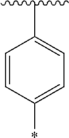 [SMILES: *c1ccc(*)cc1] | 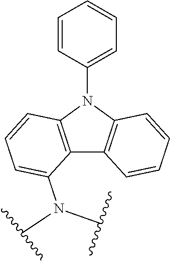 [SMILES: *N(*)c1cccc2c1c1ccccc1n2-c1ccccc1] | 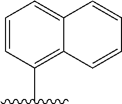 [SMILES: *c1cccc2ccccc12] |
| E7 | 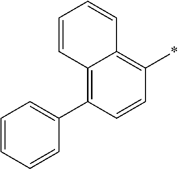 [SMILES: *c1ccc(-c2ccccc2)c2ccccc12] | 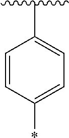 [SMILES: *c1ccc(*)cc1] | 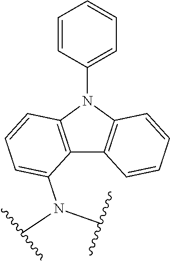 [SMILES: *N(*)c1cccc2c1c1ccccc1n2-c1ccccc1] | 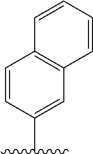 [SMILES: *c1ccc2ccccc2c1] |
| E8 | 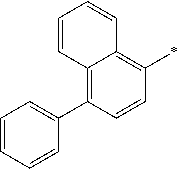 [SMILES: *c1ccc(-c2ccccc2)c2ccccc12] | 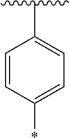 [SMILES: *c1ccc(*)cc1] | 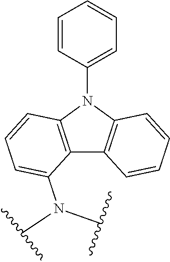 [SMILES: *N(*)c1cccc2c1c1ccccc1n2-c1ccccc1] | 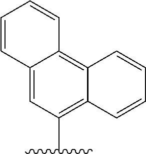 [SMILES: *c1cc2ccccc2c2ccccc12] |
| E9 | 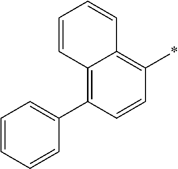 [SMILES: *c1ccc(-c2ccccc2)c2ccccc12] | 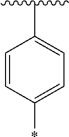 [SMILES: *c1ccc(*)cc1] | 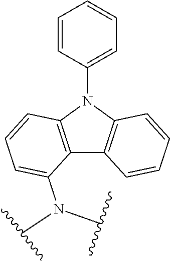 [SMILES: *N(*)c1cccc2c1c1ccccc1n2-c1ccccc1] | 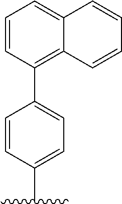 [SMILES: *c1ccc(-c2cccc3ccccc23)cc1] |
| E10 | 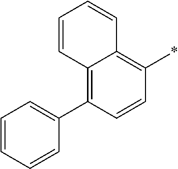 [SMILES: *c1ccc(-c2ccccc2)c2ccccc12] | 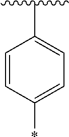 [SMILES: *c1ccc(*)cc1] | 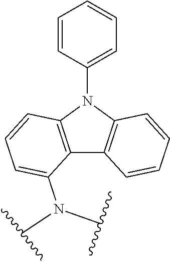 [SMILES: *N(*)c1cccc2c1c1ccccc1n2-c1ccccc1] | 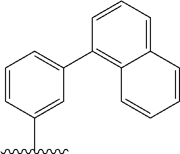 [SMILES: *c1cccc(-c2cccc3ccccc23)c1] |
| E11 | 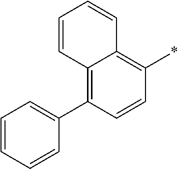 [SMILES: *c1ccc(-c2ccccc2)c2ccccc12] | 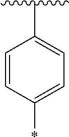 [SMILES: *c1ccc(*)cc1] | 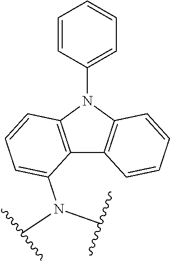 [SMILES: *N(*)c1cccc2c1c1ccccc1n2-c1ccccc1] | 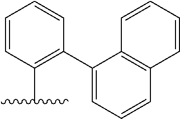 [SMILES: *c1ccccc1-c1cccc2ccccc12] |
| E12 | 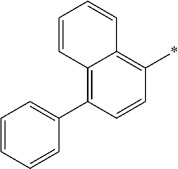 [SMILES: *c1ccc(-c2ccccc2)c2ccccc12] | 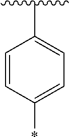 [SMILES: *c1ccc(*)cc1] | 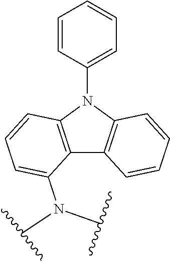 [SMILES: *N(*)c1cccc2c1c1ccccc1n2-c1ccccc1] | 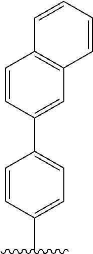 [SMILES: *c1ccc(-c2ccc3ccccc3c2)cc1] |
| E13 | 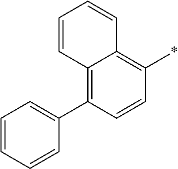 [SMILES: *c1ccc(-c2ccccc2)c2ccccc12] | 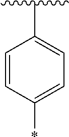 [SMILES: *c1ccc(*)cc1] | 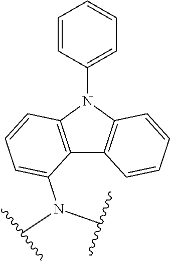 [SMILES: *N(*)c1cccc2c1c1ccccc1n2-c1ccccc1] | 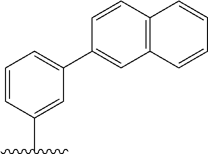 [SMILES: *c1cccc(-c2ccc3ccccc3c2)c1] |
| E14 | 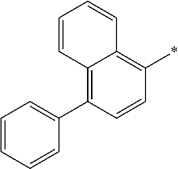 [SMILES: *c1ccc(-c2ccccc2)c2ccccc12] | 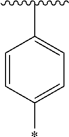 [SMILES: *c1ccc(*)cc1] | 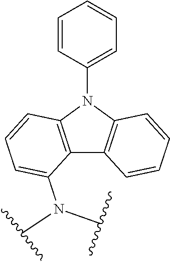 [SMILES: *N(*)c1cccc2c1c1ccccc1n2-c1ccccc1] | 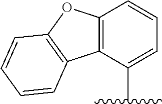 [SMILES: *c1cccc2oc3ccccc3c12] |
| E15 | 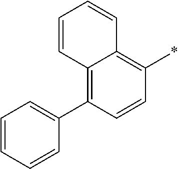 [SMILES: *c1ccc(-c2ccccc2)c2ccccc12] | 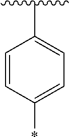 [SMILES: *c1ccc(*)cc1] | 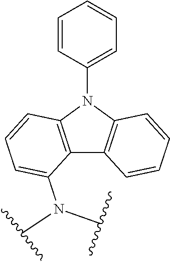 [SMILES: *N(*)c1cccc2c1c1ccccc1n2-c1ccccc1] | 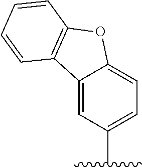 [SMILES: *c1ccc2oc3ccccc3c2c1] |
| E16 | 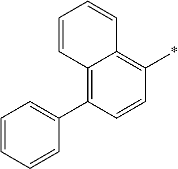 [SMILES: *c1ccc(-c2ccccc2)c2ccccc12] | 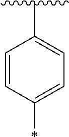 [SMILES: *c1ccc(*)cc1] | 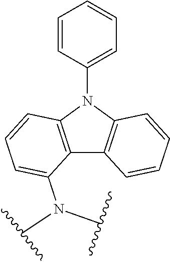 [SMILES: *N(*)c1cccc2c1c1ccccc1n2-c1ccccc1] | 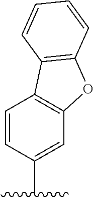 [SMILES: *c1ccc2c(c1)oc1ccccc12] |
| E17 | 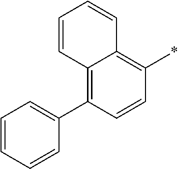 [SMILES: *c1ccc(-c2ccccc2)c2ccccc12] | 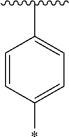 [SMILES: *c1ccc(*)cc1] | 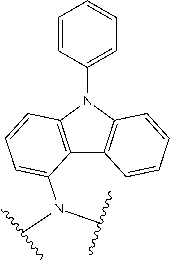 [SMILES: *N(*)c1cccc2c1c1ccccc1n2-c1ccccc1] | 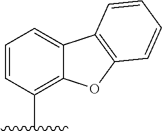 [SMILES: *c1cccc2c1oc1ccccc12] |
| E18 | 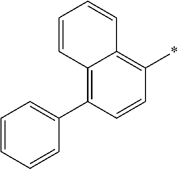 [SMILES: *c1ccc(-c2ccccc2)c2ccccc12] | 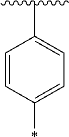 [SMILES: *c1ccc(*)cc1] | 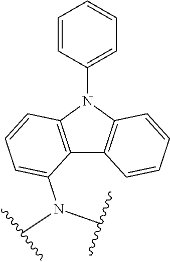 [SMILES: *N(*)c1cccc2c1c1ccccc1n2-c1ccccc1] | 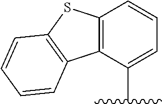 [SMILES: *c1cccc2sc3ccccc3c12] |
| E19 | 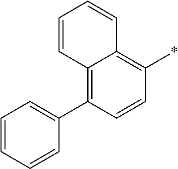 [SMILES: *c1ccc(-c2ccccc2)c2ccccc12] | 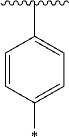 [SMILES: *c1ccc(*)cc1] | 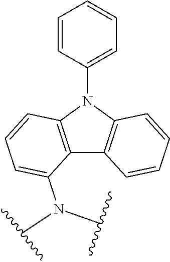 [SMILES: *N(*)c1cccc2c1c1ccccc1n2-c1ccccc1] | 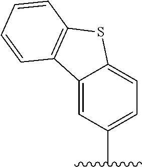 [SMILES: *c1ccc2sc3ccccc3c2c1] |
| E20 | 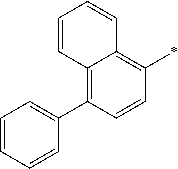 [SMILES: *c1ccc(-c2ccccc2)c2ccccc12] | 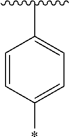 [SMILES: *c1ccc(*)cc1] | 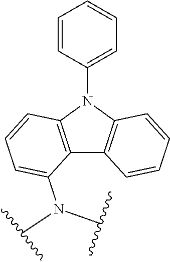 [SMILES: *N(*)c1cccc2c1c1ccccc1n2-c1ccccc1] | 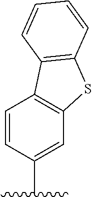 [SMILES: *c1ccc2c(c1)sc1ccccc12] |
| E21 | 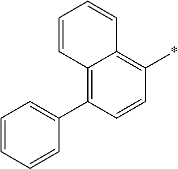 [SMILES: *c1ccc(-c2ccccc2)c2ccccc12] | 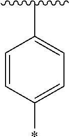 [SMILES: *c1ccc(*)cc1] | 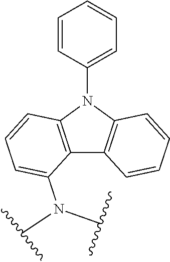 [SMILES: *N(*)c1cccc2c1c1ccccc1n2-c1ccccc1] | 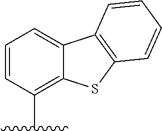 [SMILES: *c1cccc2c1sc1ccccc12] |
| E22 | 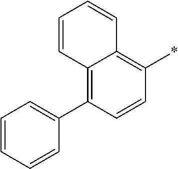 [SMILES: *c1ccc(-c2ccccc2)c2ccccc12] | 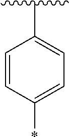 [SMILES: *c1ccc(*)cc1] | 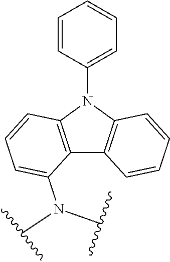 [SMILES: *N(*)c1cccc2c1c1ccccc1n2-c1ccccc1] | 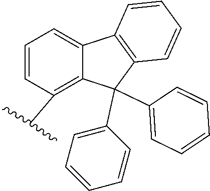 [SMILES: *c1cccc2c1C(c1ccccc1)(c1ccccc1)c1ccccc1-2] |
| E23 | 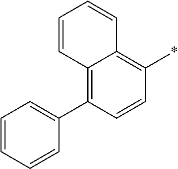 [SMILES: *c1ccc(-c2ccccc2)c2ccccc12] | 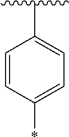 [SMILES: *c1ccc(*)cc1] | 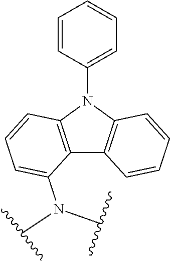 [SMILES: *N(*)c1cccc2c1c1ccccc1n2-c1ccccc1] | 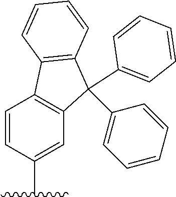 [SMILES: *c1ccc2c(c1)C(c1ccccc1)(c1ccccc1)c1ccccc1-2] |
| E24 | 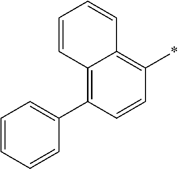 [SMILES: *c1ccc(-c2ccccc2)c2ccccc12] | 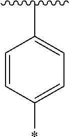 [SMILES: *c1ccc(*)cc1] | 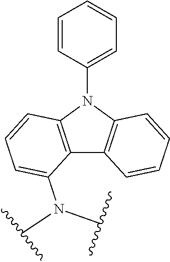 [SMILES: *N(*)c1cccc2c1c1ccccc1n2-c1ccccc1] | 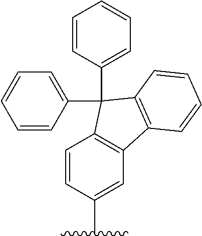 [SMILES: *c1ccc2c(c1)-c1ccccc1C2(c1ccccc1)c1ccccc1] |
| E25 | 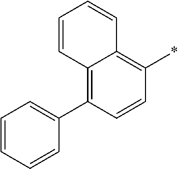 [SMILES: *c1ccc(-c2ccccc2)c2ccccc12] | 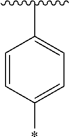 [SMILES: *c1ccc(*)cc1] | 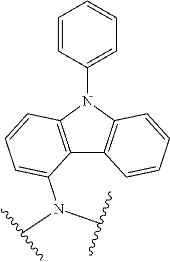 [SMILES: *N(*)c1cccc2c1c1ccccc1n2-c1ccccc1] | 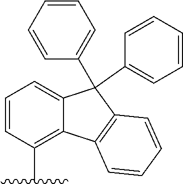 [SMILES: *c1cccc2c1-c1ccccc1C2(c1ccccc1)c1ccccc1] |
| E26 | 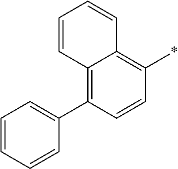 [SMILES: *c1ccc(-c2ccccc2)c2ccccc12] | 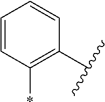 [SMILES: *c1ccccc1*] | 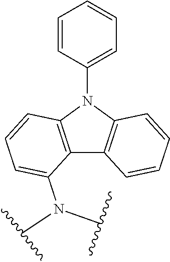 [SMILES: *N(*)c1cccc2c1c1ccccc1n2-c1ccccc1] | 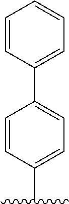 [SMILES: *c1ccc(-c2ccccc2)cc1] |
| E27 | 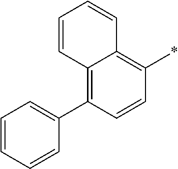 [SMILES: *c1ccc(-c2ccccc2)c2ccccc12] | 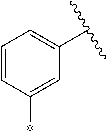 [SMILES: *c1cccc(*)c1] | 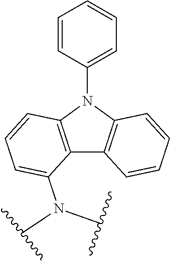 [SMILES: *N(*)c1cccc2c1c1ccccc1n2-c1ccccc1] | 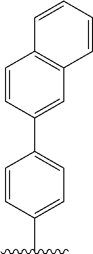 [SMILES: *c1ccc(-c2ccc3ccccc3c2)cc1] |
| E28 | 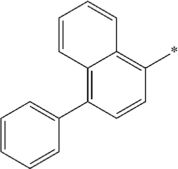 [SMILES: *c1ccc(-c2ccccc2)c2ccccc12] | 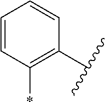 [SMILES: *c1ccccc1*] | 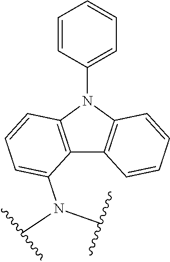 [SMILES: *N(*)c1cccc2c1c1ccccc1n2-c1ccccc1] | 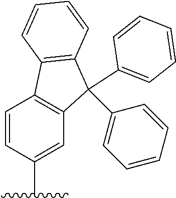 [SMILES: *c1ccc2c(c1)C(c1ccccc1)(c1ccccc1)c1ccccc1-2] |
| E29 | 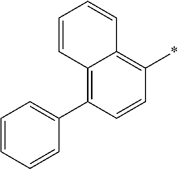 [SMILES: *c1ccc(-c2ccccc2)c2ccccc12] | 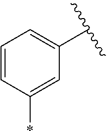 [SMILES: *c1cccc(*)c1] | 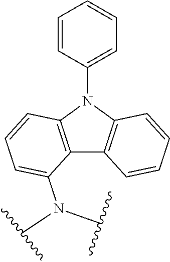 [SMILES: *N(*)c1cccc2c1c1ccccc1n2-c1ccccc1] | 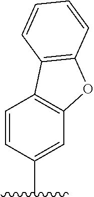 [SMILES: *c1ccc2c(c1)oc1ccccc12] |
| E30 | 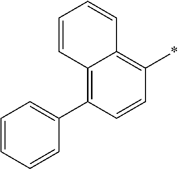 [SMILES: *c1ccc(-c2ccccc2)c2ccccc12] | 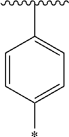 [SMILES: *c1ccc(*)cc1] | 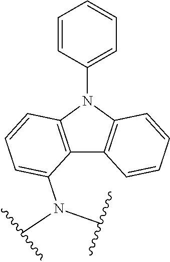 [SMILES: *N(*)c1cccc2c1c1ccccc1n2-c1ccccc1] | 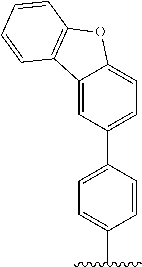 [SMILES: *c1ccc(-c2ccc3oc4ccccc4c3c2)cc1] |
| E31 | 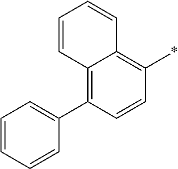 [SMILES: *c1ccc(-c2ccccc2)c2ccccc12] | 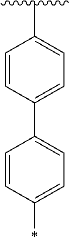 [SMILES: *c1ccc(-c2ccc(*)cc2)cc1] | 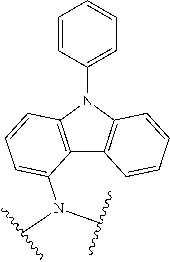 [SMILES: *N(*)c1cccc2c1c1ccccc1n2-c1ccccc1] | 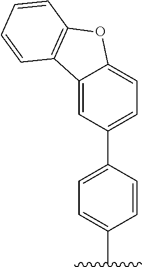 [SMILES: *c1ccc(-c2ccc3oc4ccccc4c3c2)cc1] |
| E32 | 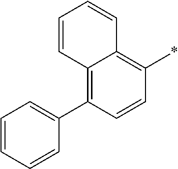 [SMILES: *c1ccc(-c2ccccc2)c2ccccc12] | 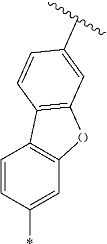 [SMILES: *c1ccc2c(c1)oc1cc(*)ccc12] | 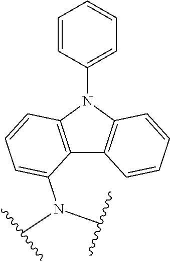 [SMILES: *N(*)c1cccc2c1c1ccccc1n2-c1ccccc1] | 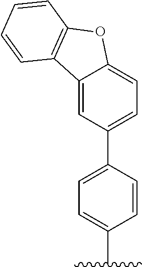 [SMILES: *c1ccc(-c2ccc3oc4ccccc4c3c2)cc1] |
| E33 | 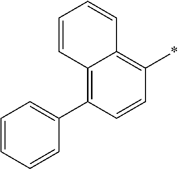 [SMILES: *c1ccc(-c2ccccc2)c2ccccc12] | 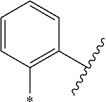 [SMILES: *c1ccccc1*] | 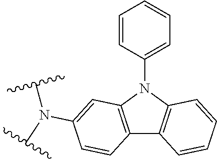 [SMILES: *N(*)c1ccc2c3ccccc3n(-c3ccccc3)c2c1] | 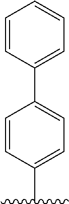 [SMILES: *c1ccc(-c2ccccc2)cc1] |
| E34 | 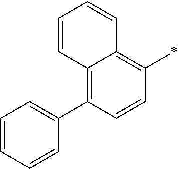 [SMILES: *c1ccc(-c2ccccc2)c2ccccc12] | 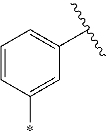 [SMILES: *c1cccc(*)c1] | 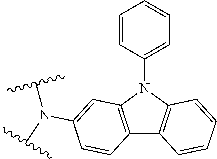 [SMILES: *N(*)c1ccc2c3ccccc3n(-c3ccccc3)c2c1] | 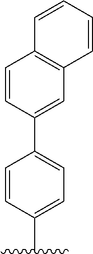 [SMILES: *c1ccc(-c2ccc3ccccc3c2)cc1] |
| E35 | 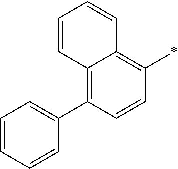 [SMILES: *c1ccc(-c2ccccc2)c2ccccc12] | 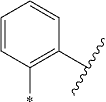 [SMILES: *c1ccccc1*] | 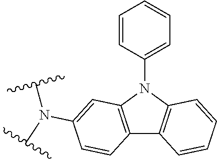 [SMILES: *N(*)c1ccc2c3ccccc3n(-c3ccccc3)c2c1] | 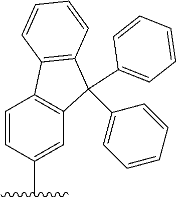 [SMILES: *c1ccc2c(c1)C(c1ccccc1)(c1ccccc1)c1ccccc1-2] |
| E36 | 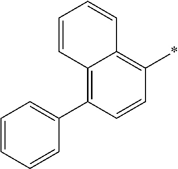 [SMILES: *c1ccc(-c2ccccc2)c2ccccc12] | 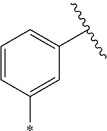 [SMILES: *c1cccc(*)c1] | 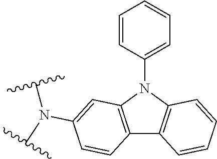 [SMILES: *N(*)c1ccc2c3ccccc3n(-c3ccccc3)c2c1] | 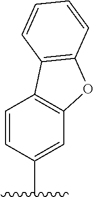 [SMILES: *c1ccc2c(c1)oc1ccccc12] |
| E37 | 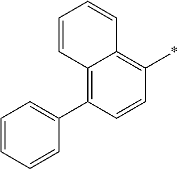 [SMILES: *c1ccc(-c2ccccc2)c2ccccc12] | 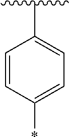 [SMILES: *c1ccc(*)cc1] | 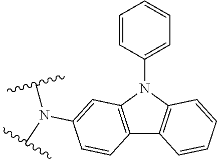 [SMILES: *N(*)c1ccc2c3ccccc3n(-c3ccccc3)c2c1] | 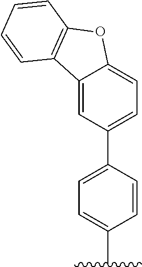 [SMILES: *c1ccc(-c2ccc3oc4ccccc4c3c2)cc1] |
| E38 | 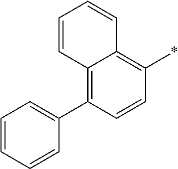 [SMILES: *c1ccc(-c2ccccc2)c2ccccc12] | 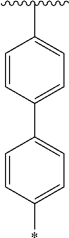 [SMILES: *c1ccc(-c2ccc(*)cc2)cc1] | 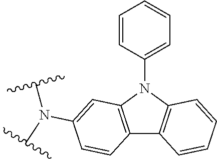 [SMILES: *N(*)c1ccc2c3ccccc3n(-c3ccccc3)c2c1] | 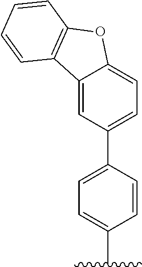 [SMILES: *c1ccc(-c2ccc3oc4ccccc4c3c2)cc1] |
| E39 | 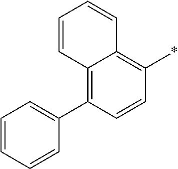 [SMILES: *c1ccc(-c2ccccc2)c2ccccc12] | 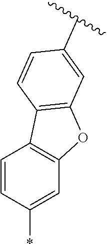 [SMILES: *c1ccc2c(c1)oc1cc(*)ccc12] | 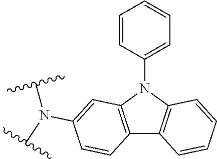 [SMILES: *N(*)c1ccc2c3ccccc3n(-c3ccccc3)c2c1] | 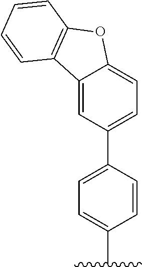 [SMILES: *c1ccc(-c2ccc3oc4ccccc4c3c2)cc1] |
| F1 | 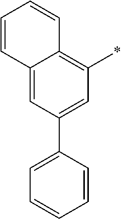 [SMILES: *c1cc(-c2ccccc2)cc2ccccc12] | 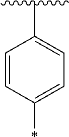 [SMILES: *c1ccc(*)cc1] | 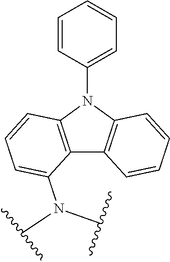 [SMILES: *N(*)c1cccc2c1c1ccccc1n2-c1ccccc1] | 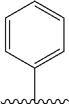 [SMILES: *c1ccccc1] |
| F2 | 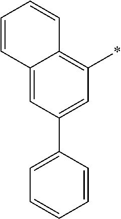 [SMILES: *c1cc(-c2ccccc2)cc2ccccc12] | 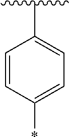 [SMILES: *c1ccc(*)cc1] | 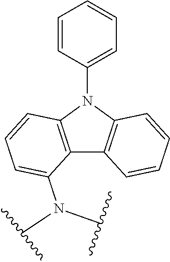 [SMILES: *N(*)c1cccc2c1c1ccccc1n2-c1ccccc1] | 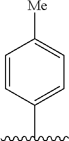 [SMILES: *c1ccc(C)cc1] |
| F3 | 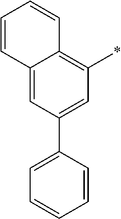 [SMILES: *c1cc(-c2ccccc2)cc2ccccc12] | 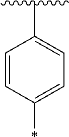 [SMILES: *c1ccc(*)cc1] | 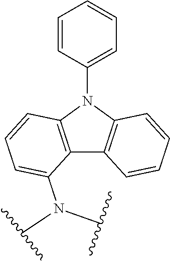 [SMILES: *N(*)c1cccc2c1c1ccccc1n2-c1ccccc1] | 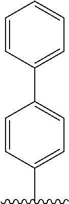 [SMILES: *c1ccc(-c2ccccc2)cc1] |
| F4 | 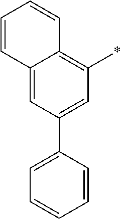 [SMILES: *c1cc(-c2ccccc2)cc2ccccc12] | 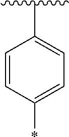 [SMILES: *c1ccc(*)cc1] | 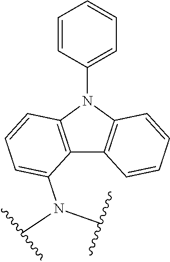 [SMILES: *N(*)c1cccc2c1c1ccccc1n2-c1ccccc1] | 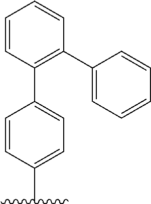 [SMILES: *c1ccc(-c2ccccc2-c2ccccc2)cc1] |
| F5 | 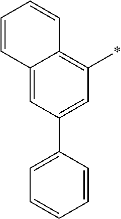 [SMILES: *c1cc(-c2ccccc2)cc2ccccc12] | 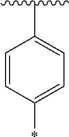 [SMILES: *c1ccc(*)cc1] | 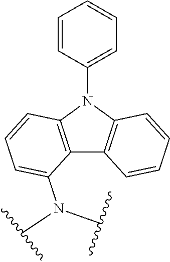 [SMILES: *N(*)c1cccc2c1c1ccccc1n2-c1ccccc1] | 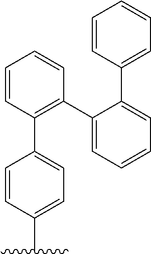 [SMILES: *c1ccc(-c2ccccc2-c2ccccc2-c2ccccc2)cc1] |
| F6 | 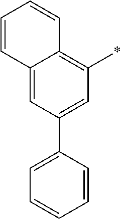 [SMILES: *c1cc(-c2ccccc2)cc2ccccc12] | 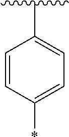 [SMILES: *c1ccc(*)cc1] | 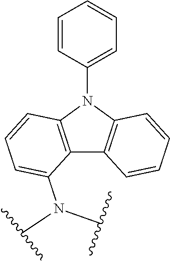 [SMILES: *N(*)c1cccc2c1c1ccccc1n2-c1ccccc1] | 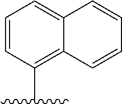 [SMILES: *c1cccc2ccccc12] |
| F7 | 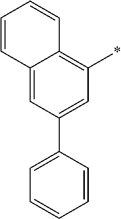 [SMILES: *c1cc(-c2ccccc2)cc2ccccc12] | 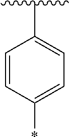 [SMILES: *c1ccc(*)cc1] | 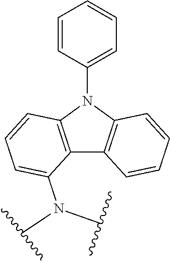 [SMILES: *N(*)c1cccc2c1c1ccccc1n2-c1ccccc1] | 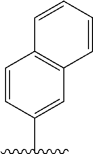 [SMILES: *c1ccc2ccccc2c1] |
| F8 | 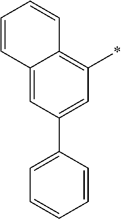 [SMILES: *c1cc(-c2ccccc2)cc2ccccc12] | 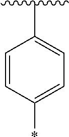 [SMILES: *c1ccc(*)cc1] | 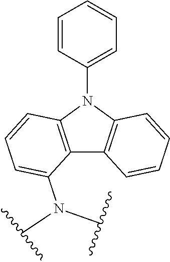 [SMILES: *N(*)c1cccc2c1c1ccccc1n2-c1ccccc1] | 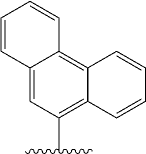 [SMILES: *c1cc2ccccc2c2ccccc12] |
| F9 | 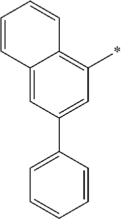 [SMILES: *c1cc(-c2ccccc2)cc2ccccc12] | 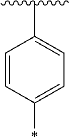 [SMILES: *c1ccc(*)cc1] | 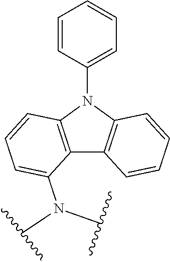 [SMILES: *N(*)c1cccc2c1c1ccccc1n2-c1ccccc1] | 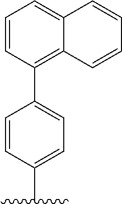 [SMILES: *c1ccc(-c2cccc3ccccc23)cc1] |
| F10 | 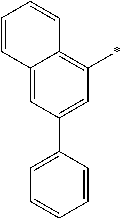 [SMILES: *c1cc(-c2ccccc2)cc2ccccc12] | 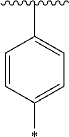 [SMILES: *c1ccc(*)cc1] | 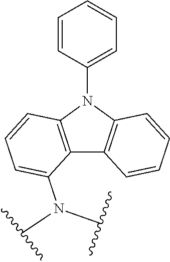 [SMILES: *N(*)c1cccc2c1c1ccccc1n2-c1ccccc1] | 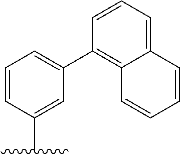 [SMILES: *c1cccc(-c2cccc3ccccc23)c1] |
| F11 | 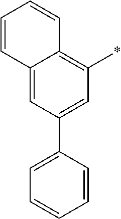 [SMILES: *c1cc(-c2ccccc2)cc2ccccc12] | 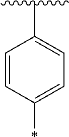 [SMILES: *c1ccc(*)cc1] | 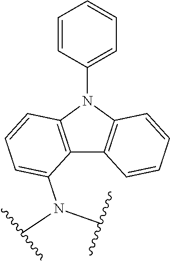 [SMILES: *N(*)c1cccc2c1c1ccccc1n2-c1ccccc1] | 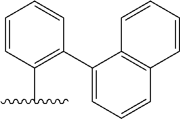 [SMILES: *c1ccccc1-c1cccc2ccccc12] |
| F12 | 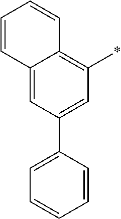 [SMILES: *c1cc(-c2ccccc2)cc2ccccc12] | 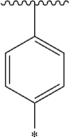 [SMILES: *c1ccc(*)cc1] | 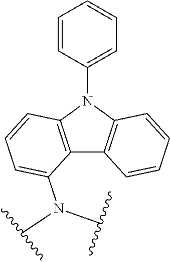 [SMILES: *N(*)c1cccc2c1c1ccccc1n2-c1ccccc1] | 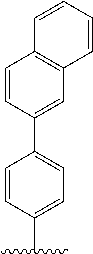 [SMILES: *c1ccc(-c2ccc3ccccc3c2)cc1] |
| F13 | 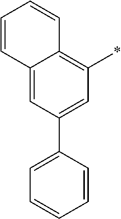 [SMILES: *c1cc(-c2ccccc2)cc2ccccc12] | 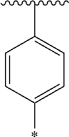 [SMILES: *c1ccc(*)cc1] | 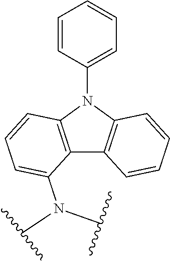 [SMILES: *N(*)c1cccc2c1c1ccccc1n2-c1ccccc1] | 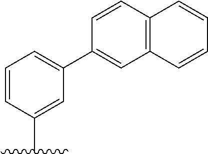 [SMILES: *c1cccc(-c2ccc3ccccc3c2)c1] |
| F14 | 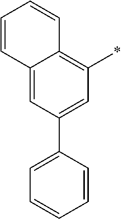 [SMILES: *c1cc(-c2ccccc2)cc2ccccc12] | 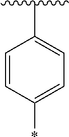 [SMILES: *c1ccc(*)cc1] | 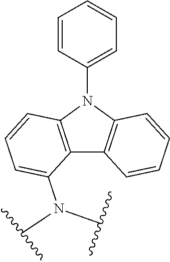 [SMILES: *N(*)c1cccc2c1c1ccccc1n2-c1ccccc1] | 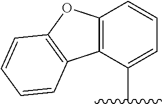 [SMILES: *c1cccc2oc3ccccc3c12] |
| F15 | 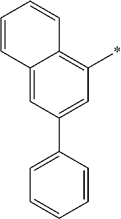 [SMILES: *c1cc(-c2ccccc2)cc2ccccc12] | 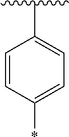 [SMILES: *c1ccc(*)cc1] | 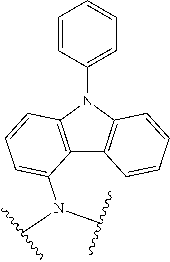 [SMILES: *N(*)c1cccc2c1c1ccccc1n2-c1ccccc1] | 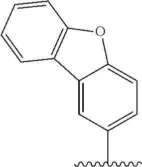 [SMILES: *c1ccc2oc3ccccc3c2c1] |
| F16 | 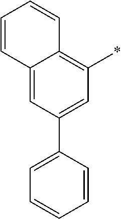 [SMILES: *c1cc(-c2ccccc2)cc2ccccc12] | 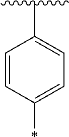 [SMILES: *c1ccc(*)cc1] | 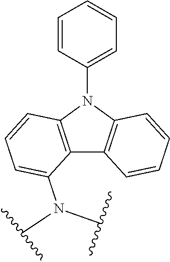 [SMILES: *N(*)c1cccc2c1c1ccccc1n2-c1ccccc1] | 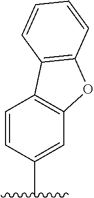 [SMILES: *c1ccc2c(c1)oc1ccccc12] |
| F17 | 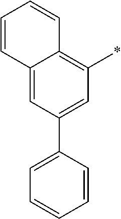 [SMILES: *c1cc(-c2ccccc2)cc2ccccc12] | 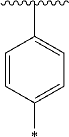 [SMILES: *c1ccc(*)cc1] | 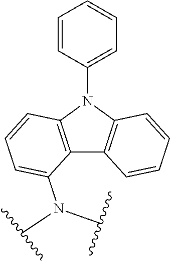 [SMILES: *N(*)c1cccc2c1c1ccccc1n2-c1ccccc1] | 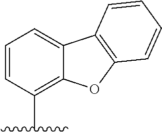 [SMILES: *c1cccc2c1oc1ccccc12] |
| F18 | 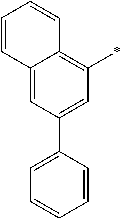 [SMILES: *c1cc(-c2ccccc2)cc2ccccc12] | 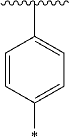 [SMILES: *c1ccc(*)cc1] | 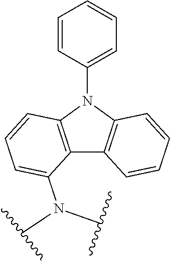 [SMILES: *N(*)c1cccc2c1c1ccccc1n2-c1ccccc1] | 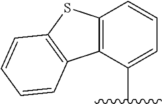 [SMILES: *c1cccc2sc3ccccc3c12] |
| F19 | 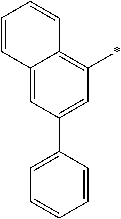 [SMILES: *c1cc(-c2ccccc2)cc2ccccc12] | 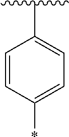 [SMILES: *c1ccc(*)cc1] | 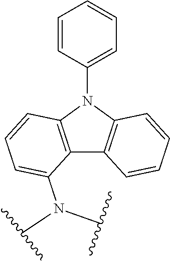 [SMILES: *N(*)c1cccc2c1c1ccccc1n2-c1ccccc1] | 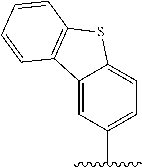 [SMILES: *c1ccc2sc3ccccc3c2c1] |
| F20 | 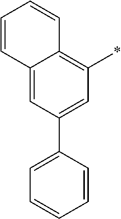 [SMILES: *c1cc(-c2ccccc2)cc2ccccc12] | 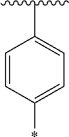 [SMILES: *c1ccc(*)cc1] | 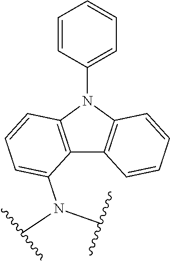 [SMILES: *N(*)c1cccc2c1c1ccccc1n2-c1ccccc1] | 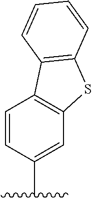 [SMILES: *c1ccc2c(c1)sc1ccccc12] |
| F21 | 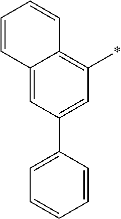 [SMILES: *c1cc(-c2ccccc2)cc2ccccc12] | 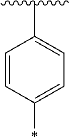 [SMILES: *c1ccc(*)cc1] | 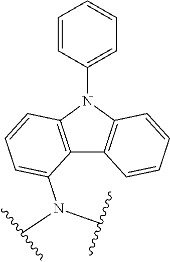 [SMILES: *N(*)c1cccc2c1c1ccccc1n2-c1ccccc1] | 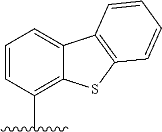 [SMILES: *c1cccc2c1sc1ccccc12] |
| F22 | 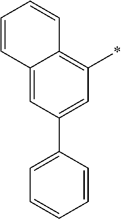 [SMILES: *c1cc(-c2ccccc2)cc2ccccc12] | 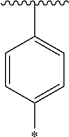 [SMILES: *c1ccc(*)cc1] | 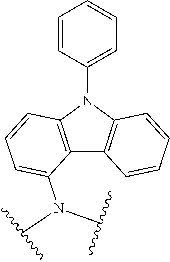 [SMILES: *N(*)c1cccc2c1c1ccccc1n2-c1ccccc1] | 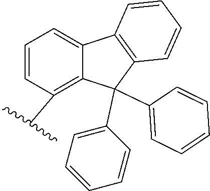 [SMILES: *c1cccc2c1C(c1ccccc1)(c1ccccc1)c1ccccc1-2] |
| F23 | 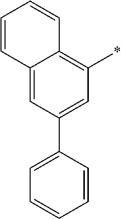 [SMILES: *c1cc(-c2ccccc2)cc2ccccc12] | 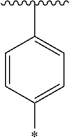 [SMILES: *c1ccc(*)cc1] | 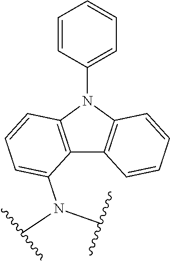 [SMILES: *N(*)c1cccc2c1c1ccccc1n2-c1ccccc1] | 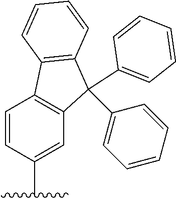 [SMILES: *c1ccc2c(c1)C(c1ccccc1)(c1ccccc1)c1ccccc1-2] |
| F24 | 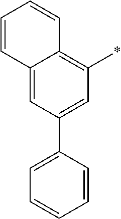 [SMILES: *c1cc(-c2ccccc2)cc2ccccc12] | 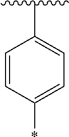 [SMILES: *c1ccc(*)cc1] | 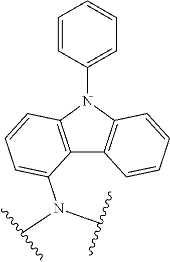 [SMILES: *N(*)c1cccc2c1c1ccccc1n2-c1ccccc1] | 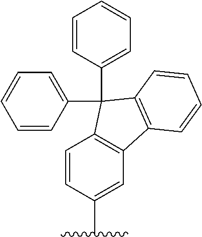 [SMILES: *c1ccc2c(c1)-c1ccccc1C2(c1ccccc1)c1ccccc1] |
| F25 | 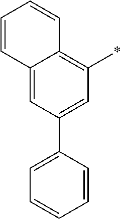 [SMILES: *c1cc(-c2ccccc2)cc2ccccc12] | 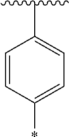 [SMILES: *c1ccc(*)cc1] | 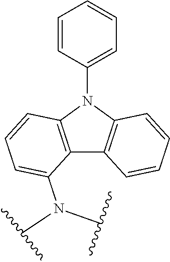 [SMILES: *N(*)c1cccc2c1c1ccccc1n2-c1ccccc1] | 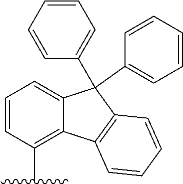 [SMILES: *c1cccc2c1-c1ccccc1C2(c1ccccc1)c1ccccc1] |
| F26 | 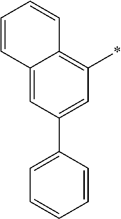 [SMILES: *c1cc(-c2ccccc2)cc2ccccc12] | 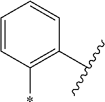 [SMILES: *c1ccccc1*] | 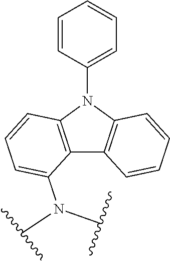 [SMILES: *N(*)c1cccc2c1c1ccccc1n2-c1ccccc1] | 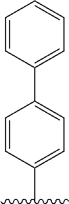 [SMILES: *c1ccc(-c2ccccc2)cc1] |
| F27 | 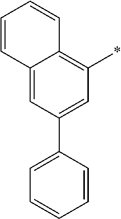 [SMILES: *c1cc(-c2ccccc2)cc2ccccc12] | 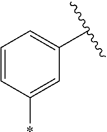 [SMILES: *c1cccc(*)c1] | 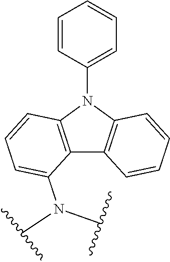 [SMILES: *N(*)c1cccc2c1c1ccccc1n2-c1ccccc1] | 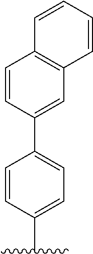 [SMILES: *c1ccc(-c2ccc3ccccc3c2)cc1] |
| F28 | 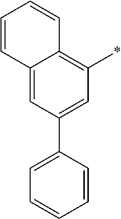 [SMILES: *c1cc(-c2ccccc2)cc2ccccc12] | 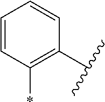 [SMILES: *c1ccccc1*] | 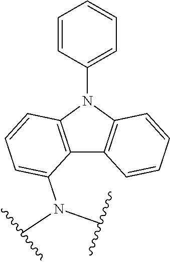 [SMILES: *N(*)c1cccc2c1c1ccccc1n2-c1ccccc1] | 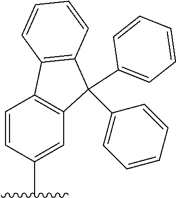 [SMILES: *c1ccc2c(c1)C(c1ccccc1)(c1ccccc1)c1ccccc1-2] |
| F29 | 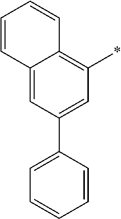 [SMILES: *c1cc(-c2ccccc2)cc2ccccc12] | 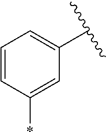 [SMILES: *c1cccc(*)c1] | 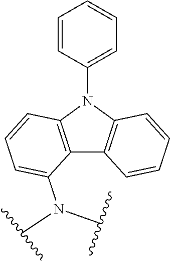 [SMILES: *N(*)c1cccc2c1c1ccccc1n2-c1ccccc1] | 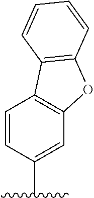 [SMILES: *c1ccc2c(c1)oc1ccccc12] |
| F30 | 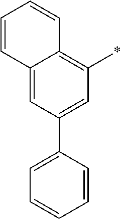 [SMILES: *c1cc(-c2ccccc2)cc2ccccc12] | 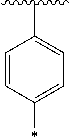 [SMILES: *c1ccc(*)cc1] | 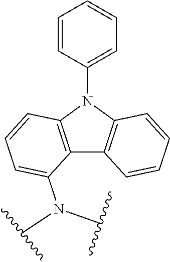 [SMILES: *N(*)c1cccc2c1c1ccccc1n2-c1ccccc1] | 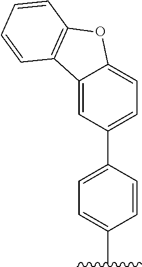 [SMILES: *c1ccc(-c2ccc3oc4ccccc4c3c2)cc1] |
| F31 | 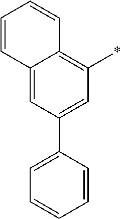 [SMILES: *c1cc(-c2ccccc2)cc2ccccc12] | 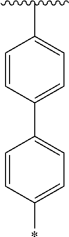 [SMILES: *c1ccc(-c2ccc(*)cc2)cc1] | 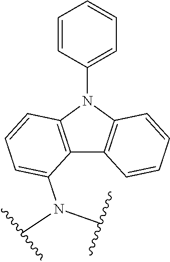 [SMILES: *N(*)c1cccc2c1c1ccccc1n2-c1ccccc1] | 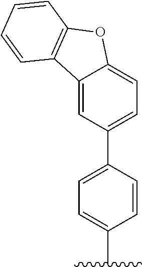 [SMILES: *c1ccc(-c2ccc3oc4ccccc4c3c2)cc1] |
| F32 | 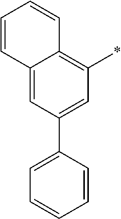 [SMILES: *c1cc(-c2ccccc2)cc2ccccc12] | 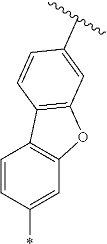 [SMILES: *c1ccc2c(c1)oc1cc(*)ccc12] | 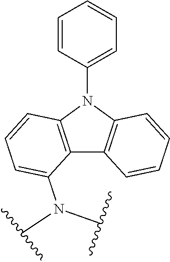 [SMILES: *N(*)c1cccc2c1c1ccccc1n2-c1ccccc1] | 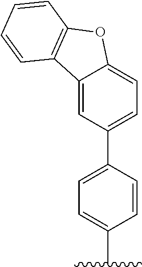 [SMILES: *c1ccc(-c2ccc3oc4ccccc4c3c2)cc1] |
| F33 | 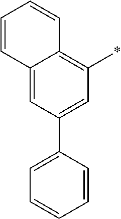 [SMILES: *c1cc(-c2ccccc2)cc2ccccc12] | 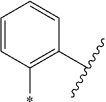 [SMILES: *c1ccccc1*] | 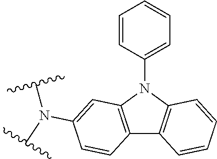 [SMILES: *N(*)c1ccc2c3ccccc3n(-c3ccccc3)c2c1] | 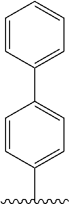 [SMILES: *c1ccc(-c2ccccc2)cc1] |
| F34 | 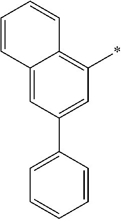 [SMILES: *c1cc(-c2ccccc2)cc2ccccc12] | 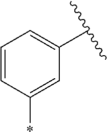 [SMILES: *c1cccc(*)c1] | 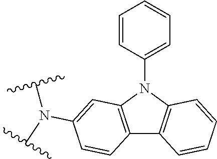 [SMILES: *N(*)c1ccc2c3ccccc3n(-c3ccccc3)c2c1] | 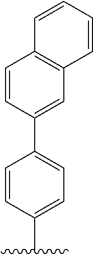 [SMILES: *c1ccc(-c2ccc3ccccc3c2)cc1] |
| F35 | 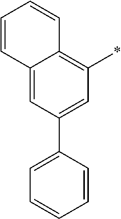 [SMILES: *c1cc(-c2ccccc2)cc2ccccc12] | 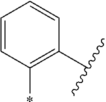 [SMILES: *c1ccccc1*] | 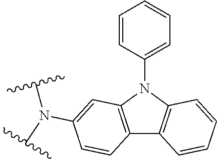 [SMILES: *N(*)c1ccc2c3ccccc3n(-c3ccccc3)c2c1] | 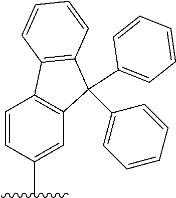 [SMILES: *c1ccc2c(c1)C(c1ccccc1)(c1ccccc1)c1ccccc1-2] |
| F36 | 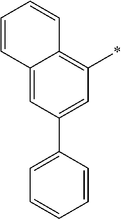 [SMILES: *c1cc(-c2ccccc2)cc2ccccc12] | 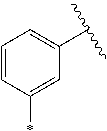 [SMILES: *c1cccc(*)c1] | 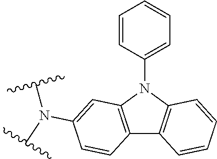 [SMILES: *N(*)c1ccc2c3ccccc3n(-c3ccccc3)c2c1] | 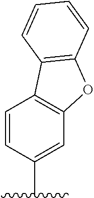 [SMILES: *c1ccc2c(c1)oc1ccccc12] |
| F37 | 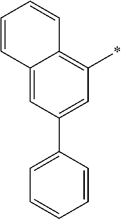 [SMILES: *c1cc(-c2ccccc2)cc2ccccc12] | 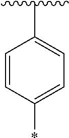 [SMILES: *c1ccc(*)cc1] | 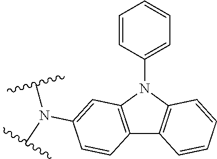 [SMILES: *N(*)c1ccc2c3ccccc3n(-c3ccccc3)c2c1] | 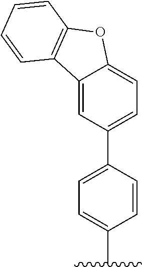 [SMILES: *c1ccc(-c2ccc3oc4ccccc4c3c2)cc1] |
| F38 | 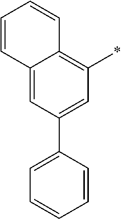 [SMILES: *c1cc(-c2ccccc2)cc2ccccc12] | 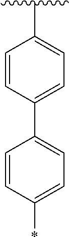 [SMILES: *c1ccc(-c2ccc(*)cc2)cc1] | 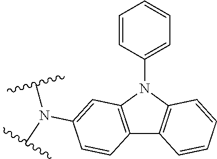 [SMILES: *N(*)c1ccc2c3ccccc3n(-c3ccccc3)c2c1] | 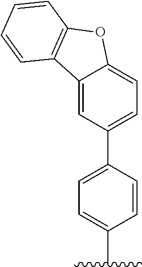 [SMILES: *c1ccc(-c2ccc3oc4ccccc4c3c2)cc1] |
| F39 | 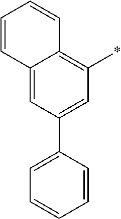 [SMILES: *c1cc(-c2ccccc2)cc2ccccc12] | 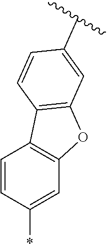 [SMILES: *c1ccc2c(c1)oc1cc(*)ccc12] | 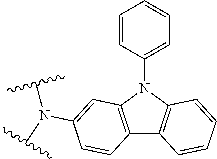 [SMILES: *N(*)c1ccc2c3ccccc3n(-c3ccccc3)c2c1] | 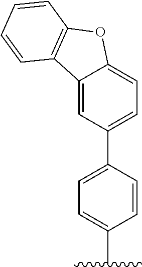 [SMILES: *c1ccc(-c2ccc3oc4ccccc4c3c2)cc1] |
| G1 | 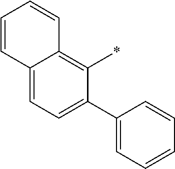 [SMILES: *c1c(-c2ccccc2)ccc2ccccc12] | 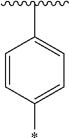 [SMILES: *c1ccc(*)cc1] | 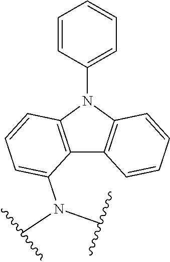 [SMILES: *N(*)c1cccc2c1c1ccccc1n2-c1ccccc1] | 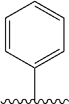 [SMILES: *c1ccccc1] |
| G2 | 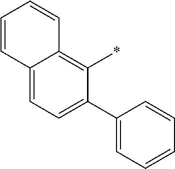 [SMILES: *c1c(-c2ccccc2)ccc2ccccc12] | 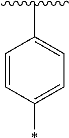 [SMILES: *c1ccc(*)cc1] | 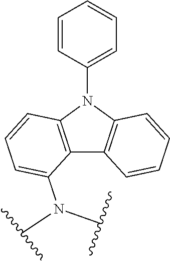 [SMILES: *N(*)c1cccc2c1c1ccccc1n2-c1ccccc1] | 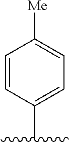 [SMILES: *c1ccc(C)cc1] |
| G3 | 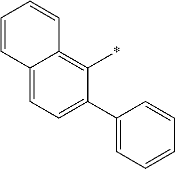 [SMILES: *c1c(-c2ccccc2)ccc2ccccc12] | 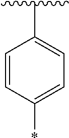 [SMILES: *c1ccc(*)cc1] | 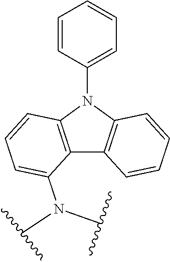 [SMILES: *N(*)c1cccc2c1c1ccccc1n2-c1ccccc1] | 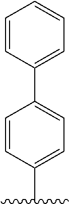 [SMILES: *c1ccc(-c2ccccc2)cc1] |
| G4 | 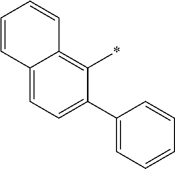 [SMILES: *c1c(-c2ccccc2)ccc2ccccc12] | 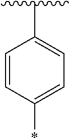 [SMILES: *c1ccc(*)cc1] | 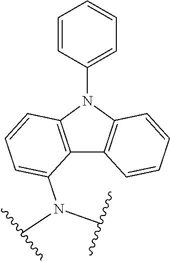 [SMILES: *N(*)c1cccc2c1c1ccccc1n2-c1ccccc1] | 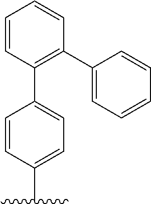 [SMILES: *c1ccc(-c2ccccc2-c2ccccc2)cc1] |
| G5 | 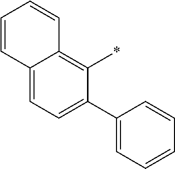 [SMILES: *c1c(-c2ccccc2)ccc2ccccc12] | 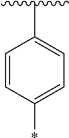 [SMILES: *c1ccc(*)cc1] | 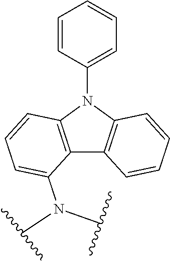 [SMILES: *N(*)c1cccc2c1c1ccccc1n2-c1ccccc1] | 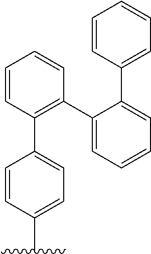 [SMILES: *c1ccc(-c2ccccc2-c2ccccc2-c2ccccc2)cc1] |
| G6 | 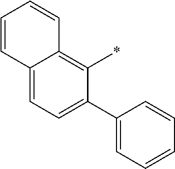 [SMILES: *c1c(-c2ccccc2)ccc2ccccc12] | 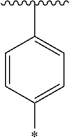 [SMILES: *c1ccc(*)cc1] | 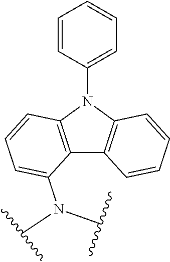 [SMILES: *N(*)c1cccc2c1c1ccccc1n2-c1ccccc1] | 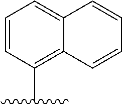 [SMILES: *c1cccc2ccccc12] |
| G7 | 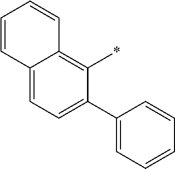 [SMILES: *c1c(-c2ccccc2)ccc2ccccc12] | 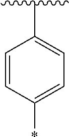 [SMILES: *c1ccc(*)cc1] | 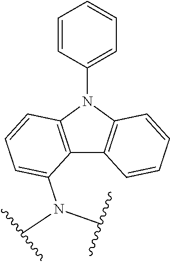 [SMILES: *N(*)c1cccc2c1c1ccccc1n2-c1ccccc1] | 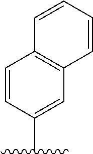 [SMILES: *c1ccc2ccccc2c1] |
| G8 | 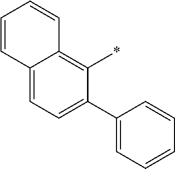 [SMILES: *c1c(-c2ccccc2)ccc2ccccc12] | 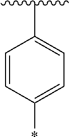 [SMILES: *c1ccc(*)cc1] | 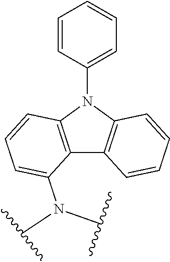 [SMILES: *N(*)c1cccc2c1c1ccccc1n2-c1ccccc1] | 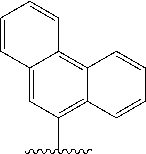 [SMILES: *c1cc2ccccc2c2ccccc12] |
| G9 | 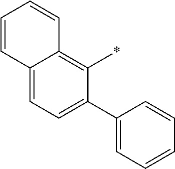 [SMILES: *c1c(-c2ccccc2)ccc2ccccc12] | 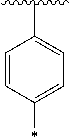 [SMILES: *c1ccc(*)cc1] | 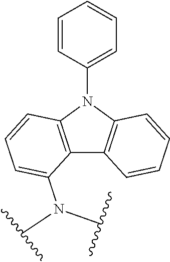 [SMILES: *N(*)c1cccc2c1c1ccccc1n2-c1ccccc1] | 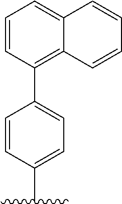 [SMILES: *c1ccc(-c2cccc3ccccc23)cc1] |
| G10 | 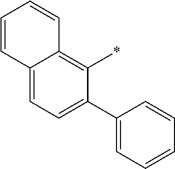 [SMILES: *c1c(-c2ccccc2)ccc2ccccc12] | 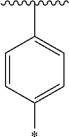 [SMILES: *c1ccc(*)cc1] | 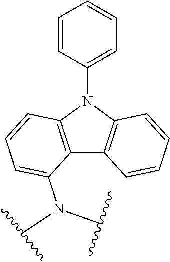 [SMILES: *N(*)c1cccc2c1c1ccccc1n2-c1ccccc1] | 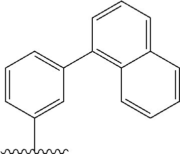 [SMILES: *c1cccc(-c2cccc3ccccc23)c1] |
| G11 | 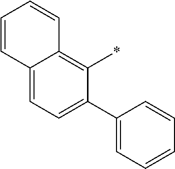 [SMILES: *c1c(-c2ccccc2)ccc2ccccc12] | 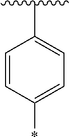 [SMILES: *c1ccc(*)cc1] | 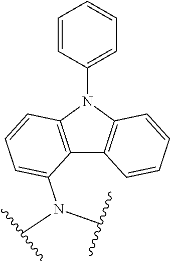 [SMILES: *N(*)c1cccc2c1c1ccccc1n2-c1ccccc1] | 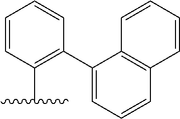 [SMILES: *c1ccccc1-c1cccc2ccccc12] |
| G12 | 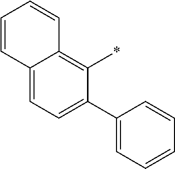 [SMILES: *c1c(-c2ccccc2)ccc2ccccc12] | 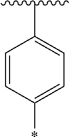 [SMILES: *c1ccc(*)cc1] | 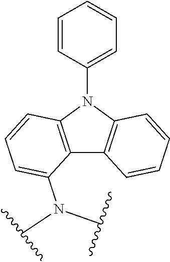 [SMILES: *N(*)c1cccc2c1c1ccccc1n2-c1ccccc1] | 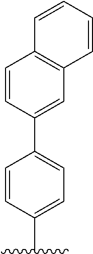 [SMILES: *c1ccc(-c2ccc3ccccc3c2)cc1] |
| G13 | 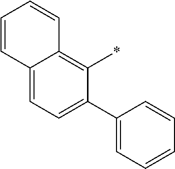 [SMILES: *c1c(-c2ccccc2)ccc2ccccc12] | 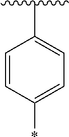 [SMILES: *c1ccc(*)cc1] | 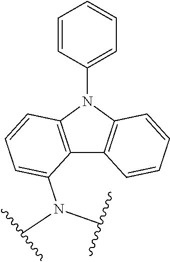 [SMILES: *N(*)c1cccc2c1c1ccccc1n2-c1ccccc1] | 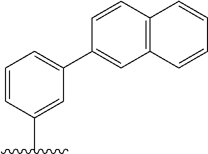 [SMILES: *c1cccc(-c2ccc3ccccc3c2)c1] |
| G14 | 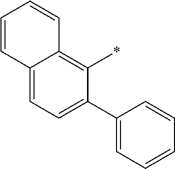 [SMILES: *c1c(-c2ccccc2)ccc2ccccc12] | 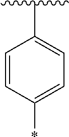 [SMILES: *c1ccc(*)cc1] | 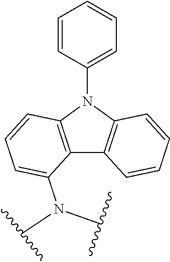 [SMILES: *N(*)c1cccc2c1c1ccccc1n2-c1ccccc1] | 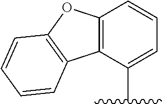 [SMILES: *c1cccc2oc3ccccc3c12] |
| G15 | 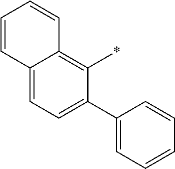 [SMILES: *c1c(-c2ccccc2)ccc2ccccc12] | 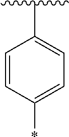 [SMILES: *c1ccc(*)cc1] | 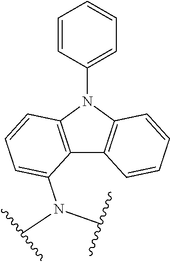 [SMILES: *N(*)c1cccc2c1c1ccccc1n2-c1ccccc1] | 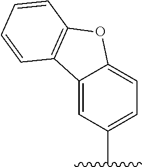 [SMILES: *c1ccc2oc3ccccc3c2c1] |
| G16 | 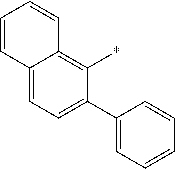 [SMILES: *c1c(-c2ccccc2)ccc2ccccc12] | 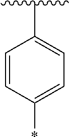 [SMILES: *c1ccc(*)cc1] | 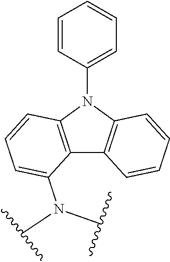 [SMILES: *N(*)c1cccc2c1c1ccccc1n2-c1ccccc1] | 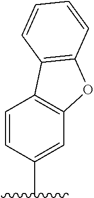 [SMILES: *c1ccc2c(c1)oc1ccccc12] |
| G17 | 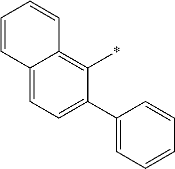 [SMILES: *c1c(-c2ccccc2)ccc2ccccc12] | 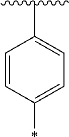 [SMILES: *c1ccc(*)cc1] | 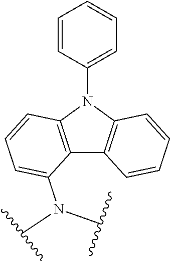 [SMILES: *N(*)c1cccc2c1c1ccccc1n2-c1ccccc1] | 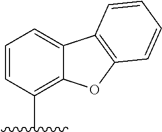 [SMILES: *c1cccc2c1oc1ccccc12] |
| G18 | 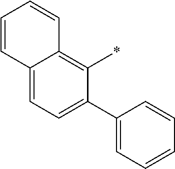 [SMILES: *c1c(-c2ccccc2)ccc2ccccc12] | 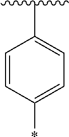 [SMILES: *c1ccc(*)cc1] | 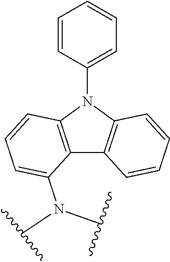 [SMILES: *N(*)c1cccc2c1c1ccccc1n2-c1ccccc1] | 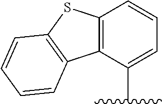 [SMILES: *c1cccc2sc3ccccc3c12] |
| G19 | 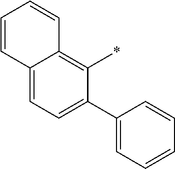 [SMILES: *c1c(-c2ccccc2)ccc2ccccc12] | 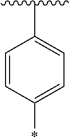 [SMILES: *c1ccc(*)cc1] | 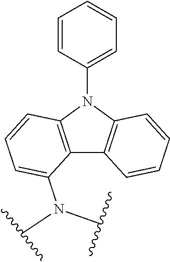 [SMILES: *N(*)c1cccc2c1c1ccccc1n2-c1ccccc1] | 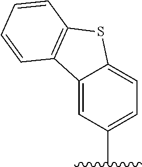 [SMILES: *c1ccc2sc3ccccc3c2c1] |
| G20 | 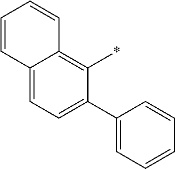 [SMILES: *c1c(-c2ccccc2)ccc2ccccc12] | 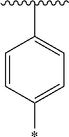 [SMILES: *c1ccc(*)cc1] | 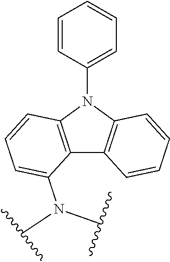 [SMILES: *N(*)c1cccc2c1c1ccccc1n2-c1ccccc1] | 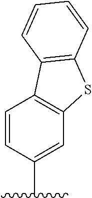 [SMILES: *c1ccc2c(c1)sc1ccccc12] |
| G21 | 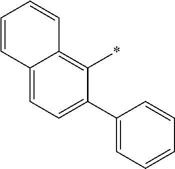 [SMILES: *c1c(-c2ccccc2)ccc2ccccc12] | 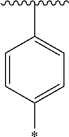 [SMILES: *c1ccc(*)cc1] | 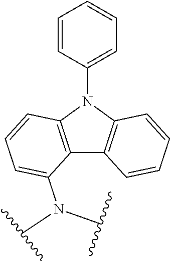 [SMILES: *N(*)c1cccc2c1c1ccccc1n2-c1ccccc1] | 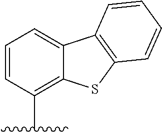 [SMILES: *c1cccc2c1sc1ccccc12] |
| G22 | 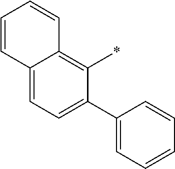 [SMILES: *c1c(-c2ccccc2)ccc2ccccc12] | 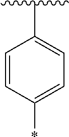 [SMILES: *c1ccc(*)cc1] | 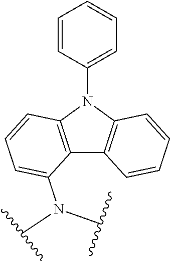 [SMILES: *N(*)c1cccc2c1c1ccccc1n2-c1ccccc1] | 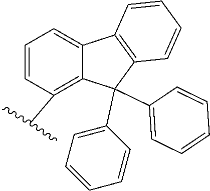 [SMILES: *c1cccc2c1C(c1ccccc1)(c1ccccc1)c1ccccc1-2] |
| G23 | 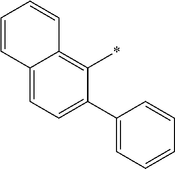 [SMILES: *c1c(-c2ccccc2)ccc2ccccc12] | 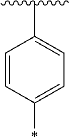 [SMILES: *c1ccc(*)cc1] | 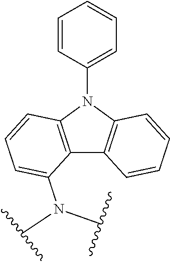 [SMILES: *N(*)c1cccc2c1c1ccccc1n2-c1ccccc1] | 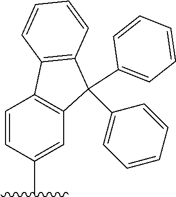 [SMILES: *c1ccc2c(c1)C(c1ccccc1)(c1ccccc1)c1ccccc1-2] |
| G24 | 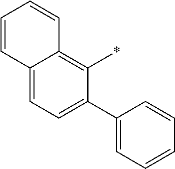 [SMILES: *c1c(-c2ccccc2)ccc2ccccc12] | 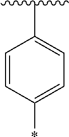 [SMILES: *c1ccc(*)cc1] | 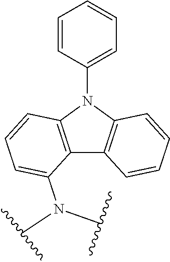 [SMILES: *N(*)c1cccc2c1c1ccccc1n2-c1ccccc1] | 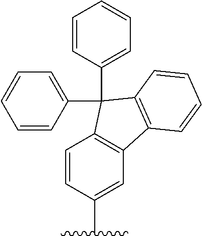 [SMILES: *c1ccc2c(c1)-c1ccccc1C2(c1ccccc1)c1ccccc1] |
| G25 | 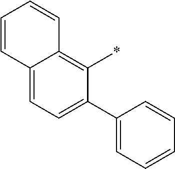 [SMILES: *c1c(-c2ccccc2)ccc2ccccc12] | 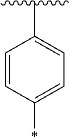 [SMILES: *c1ccc(*)cc1] | 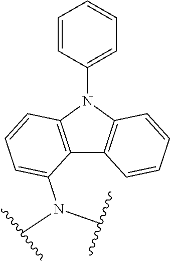 [SMILES: *N(*)c1cccc2c1c1ccccc1n2-c1ccccc1] | 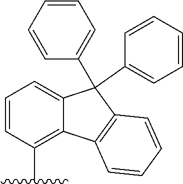 [SMILES: *c1cccc2c1-c1ccccc1C2(c1ccccc1)c1ccccc1] |
| G26 | 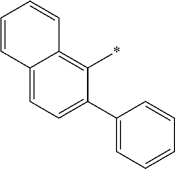 [SMILES: *c1c(-c2ccccc2)ccc2ccccc12] | 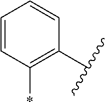 [SMILES: *c1ccccc1*] | 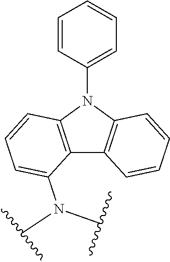 [SMILES: *N(*)c1cccc2c1c1ccccc1n2-c1ccccc1] | 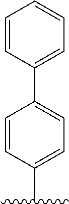 [SMILES: *c1ccc(-c2ccccc2)cc1] |
| G27 | 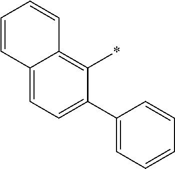 [SMILES: *c1c(-c2ccccc2)ccc2ccccc12] | 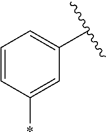 [SMILES: *c1cccc(*)c1] | 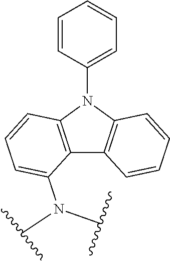 [SMILES: *N(*)c1cccc2c1c1ccccc1n2-c1ccccc1] | 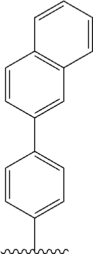 [SMILES: *c1ccc(-c2ccc3ccccc3c2)cc1] |
| G28 | 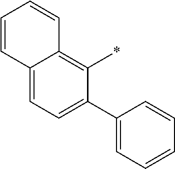 [SMILES: *c1c(-c2ccccc2)ccc2ccccc12] | 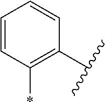 [SMILES: *c1ccccc1*] | 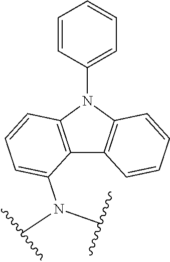 [SMILES: *N(*)c1cccc2c1c1ccccc1n2-c1ccccc1] | 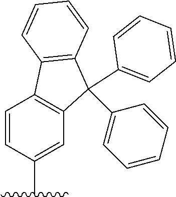 [SMILES: *c1ccc2c(c1)C(c1ccccc1)(c1ccccc1)c1ccccc1-2] |
| G29 | 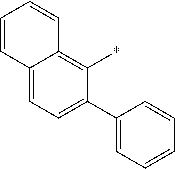 [SMILES: *c1c(-c2ccccc2)ccc2ccccc12] | 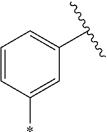 [SMILES: *c1cccc(*)c1] | 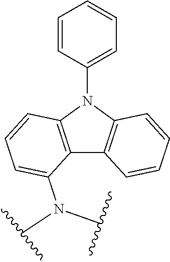 [SMILES: *N(*)c1cccc2c1c1ccccc1n2-c1ccccc1] | 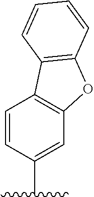 [SMILES: *c1ccc2c(c1)oc1ccccc12] |
| G30 | 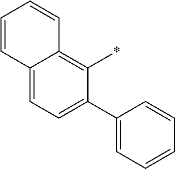 [SMILES: *c1c(-c2ccccc2)ccc2ccccc12] | 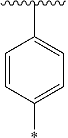 [SMILES: *c1ccc(*)cc1] | 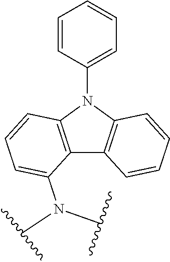 [SMILES: *N(*)c1cccc2c1c1ccccc1n2-c1ccccc1] | 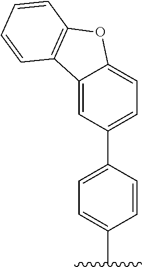 [SMILES: *c1ccc(-c2ccc3oc4ccccc4c3c2)cc1] |
| G31 | 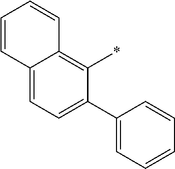 [SMILES: *c1c(-c2ccccc2)ccc2ccccc12] | 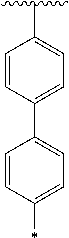 [SMILES: *c1ccc(-c2ccc(*)cc2)cc1] | 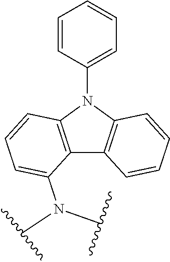 [SMILES: *N(*)c1cccc2c1c1ccccc1n2-c1ccccc1] | 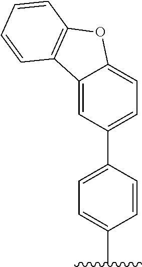 [SMILES: *c1ccc(-c2ccc3oc4ccccc4c3c2)cc1] |
| G32 | 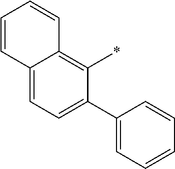 [SMILES: *c1c(-c2ccccc2)ccc2ccccc12] | 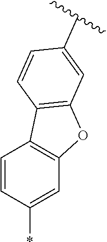 [SMILES: *c1ccc2c(c1)oc1cc(*)ccc12] | 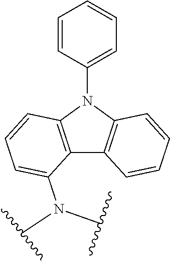 [SMILES: *N(*)c1cccc2c1c1ccccc1n2-c1ccccc1] | 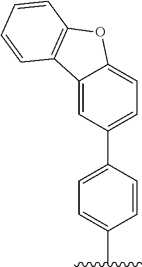 [SMILES: *c1ccc(-c2ccc3oc4ccccc4c3c2)cc1] |
| G33 | 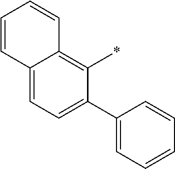 [SMILES: *c1c(-c2ccccc2)ccc2ccccc12] | 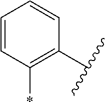 [SMILES: *c1ccccc1*] | 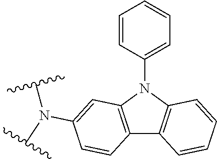 [SMILES: *N(*)c1ccc2c3ccccc3n(-c3ccccc3)c2c1] | 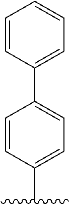 [SMILES: *c1ccc(-c2ccccc2)cc1] |
| G34 | 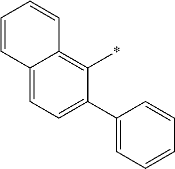 [SMILES: *c1c(-c2ccccc2)ccc2ccccc12] | 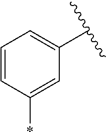 [SMILES: *c1cccc(*)c1] | 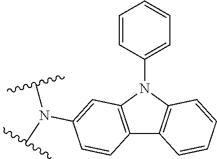 [SMILES: *N(*)c1ccc2c3ccccc3n(-c3ccccc3)c2c1] | 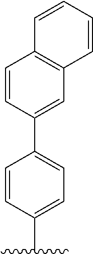 [SMILES: *c1ccc(-c2ccc3ccccc3c2)cc1] |
| G35 | 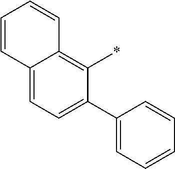 [SMILES: *c1c(-c2ccccc2)ccc2ccccc12] | 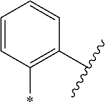 [SMILES: *c1ccccc1*] | 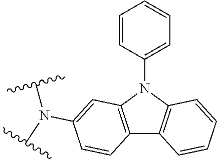 [SMILES: *N(*)c1ccc2c3ccccc3n(-c3ccccc3)c2c1] | 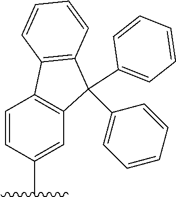 [SMILES: *c1ccc2c(c1)C(c1ccccc1)(c1ccccc1)c1ccccc1-2] |
| G36 | 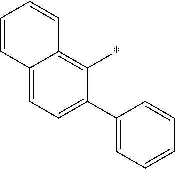 [SMILES: *c1c(-c2ccccc2)ccc2ccccc12] | 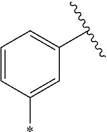 [SMILES: *c1cccc(*)c1] | 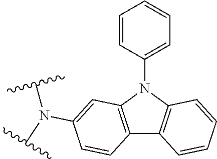 [SMILES: *N(*)c1ccc2c3ccccc3n(-c3ccccc3)c2c1] | 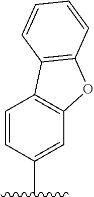 [SMILES: *c1ccc2c(c1)oc1ccccc12] |
| G37 | 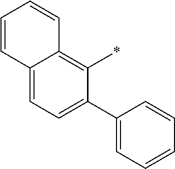 [SMILES: *c1c(-c2ccccc2)ccc2ccccc12] | 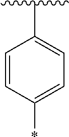 [SMILES: *c1ccc(*)cc1] | 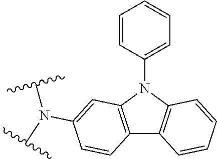 [SMILES: *N(*)c1ccc2c3ccccc3n(-c3ccccc3)c2c1] | 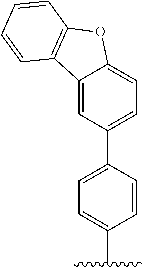 [SMILES: *c1ccc(-c2ccc3oc4ccccc4c3c2)cc1] |
| G38 | 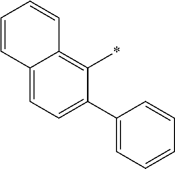 [SMILES: *c1c(-c2ccccc2)ccc2ccccc12] | 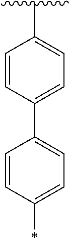 [SMILES: *c1ccc(-c2ccc(*)cc2)cc1] | 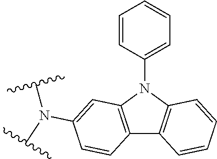 [SMILES: *N(*)c1ccc2c3ccccc3n(-c3ccccc3)c2c1] | 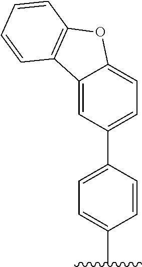 [SMILES: *c1ccc(-c2ccc3oc4ccccc4c3c2)cc1] |
| G39 | 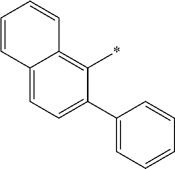 [SMILES: *c1c(-c2ccccc2)ccc2ccccc12] | 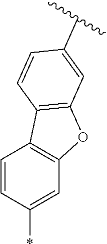 [SMILES: *c1ccc2c(c1)oc1cc(*)ccc12] | 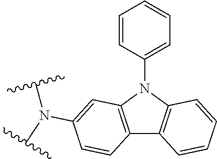 [SMILES: *N(*)c1ccc2c3ccccc3n(-c3ccccc3)c2c1] | 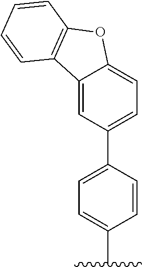 [SMILES: *c1ccc(-c2ccc3oc4ccccc4c3c2)cc1] |
| H1 | 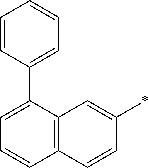 [SMILES: *c1ccc2cccc(-c3ccccc3)c2c1] | 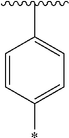 [SMILES: *c1ccc(*)cc1] | 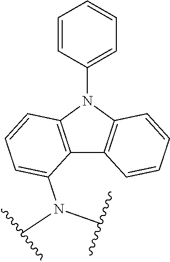 [SMILES: *N(*)c1cccc2c1c1ccccc1n2-c1ccccc1] | 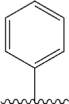 [SMILES: *c1ccccc1] |
| H2 | 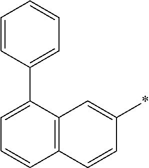 [SMILES: *c1ccc2cccc(-c3ccccc3)c2c1] | 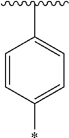 [SMILES: *c1ccc(*)cc1] | 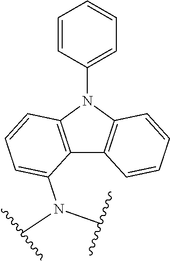 [SMILES: *N(*)c1cccc2c1c1ccccc1n2-c1ccccc1] | 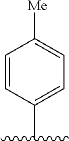 [SMILES: *c1ccc(C)cc1] |
| H3 | 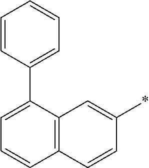 [SMILES: *c1ccc2cccc(-c3ccccc3)c2c1] | 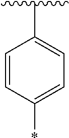 [SMILES: *c1ccc(*)cc1] | 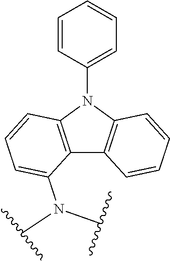 [SMILES: *N(*)c1cccc2c1c1ccccc1n2-c1ccccc1] | 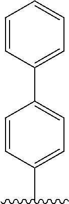 [SMILES: *c1ccc(-c2ccccc2)cc1] |
| H4 | 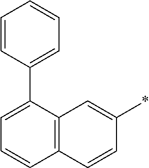 [SMILES: *c1ccc2cccc(-c3ccccc3)c2c1] | 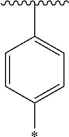 [SMILES: *c1ccc(*)cc1] | 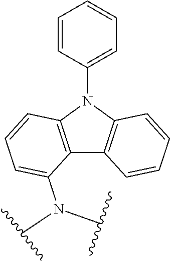 [SMILES: *N(*)c1cccc2c1c1ccccc1n2-c1ccccc1] | 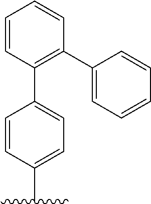 [SMILES: *c1ccc(-c2ccccc2-c2ccccc2)cc1] |
| H5 | 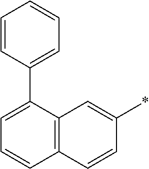 [SMILES: *c1ccc2cccc(-c3ccccc3)c2c1] | 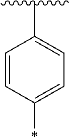 [SMILES: *c1ccc(*)cc1] | 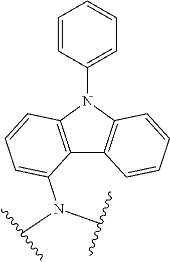 [SMILES: *N(*)c1cccc2c1c1ccccc1n2-c1ccccc1] | 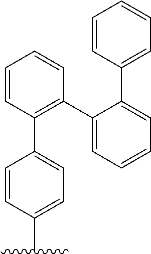 [SMILES: *c1ccc(-c2ccccc2-c2ccccc2-c2ccccc2)cc1] |
| H6 | 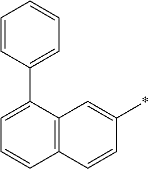 [SMILES: *c1ccc2cccc(-c3ccccc3)c2c1] | 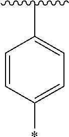 [SMILES: *c1ccc(*)cc1] | 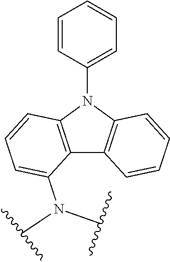 [SMILES: *N(*)c1cccc2c1c1ccccc1n2-c1ccccc1] | 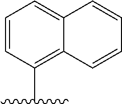 [SMILES: *c1cccc2ccccc12] |
| H7 | 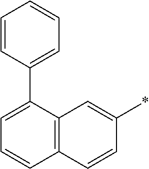 [SMILES: *c1ccc2cccc(-c3ccccc3)c2c1] | 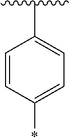 [SMILES: *c1ccc(*)cc1] | 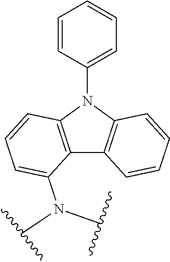 [SMILES: *N(*)c1cccc2c1c1ccccc1n2-c1ccccc1] | 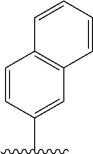 [SMILES: *c1ccc2ccccc2c1] |
| H8 | 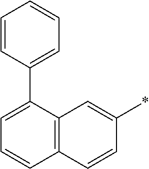 [SMILES: *c1ccc2cccc(-c3ccccc3)c2c1] | 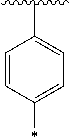 [SMILES: *c1ccc(*)cc1] | 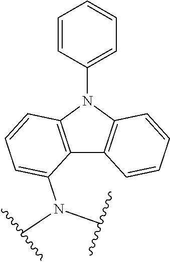 [SMILES: *N(*)c1cccc2c1c1ccccc1n2-c1ccccc1] | 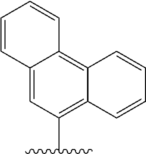 [SMILES: *c1cc2ccccc2c2ccccc12] |
| H9 | 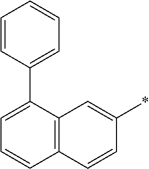 [SMILES: *c1ccc2cccc(-c3ccccc3)c2c1] | 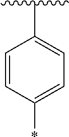 [SMILES: *c1ccc(*)cc1] | 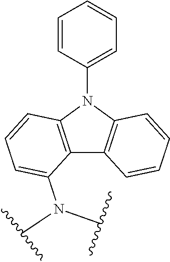 [SMILES: *N(*)c1cccc2c1c1ccccc1n2-c1ccccc1] | 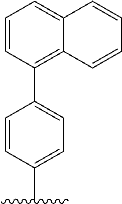 [SMILES: *c1ccc(-c2cccc3ccccc23)cc1] |
| H10 | 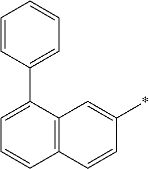 [SMILES: *c1ccc2cccc(-c3ccccc3)c2c1] | 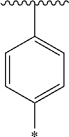 [SMILES: *c1ccc(*)cc1] | 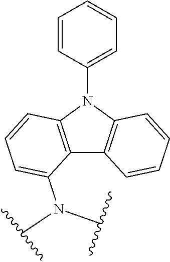 [SMILES: *N(*)c1cccc2c1c1ccccc1n2-c1ccccc1] | 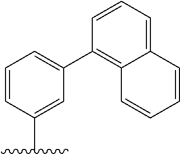 [SMILES: *c1cccc(-c2cccc3ccccc23)c1] |
| H11 | 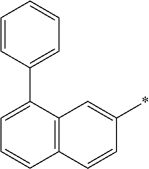 [SMILES: *c1ccc2cccc(-c3ccccc3)c2c1] | 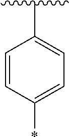 [SMILES: *c1ccc(*)cc1] | 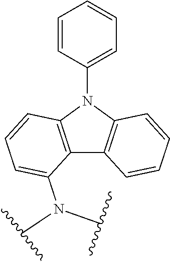 [SMILES: *N(*)c1cccc2c1c1ccccc1n2-c1ccccc1] | 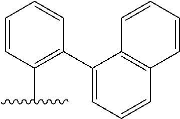 [SMILES: *c1ccccc1-c1cccc2ccccc12] |
| H12 | 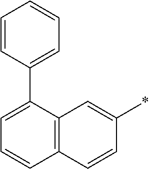 [SMILES: *c1ccc2cccc(-c3ccccc3)c2c1] | 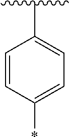 [SMILES: *c1ccc(*)cc1] | 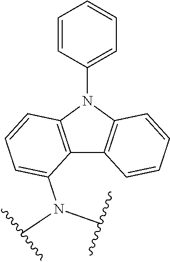 [SMILES: *N(*)c1cccc2c1c1ccccc1n2-c1ccccc1] | 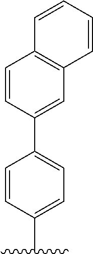 [SMILES: *c1ccc(-c2ccc3ccccc3c2)cc1] |
| H13 | 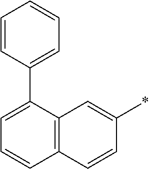 [SMILES: *c1ccc2cccc(-c3ccccc3)c2c1] | 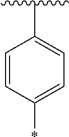 [SMILES: *c1ccc(*)cc1] | 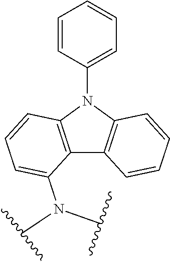 [SMILES: *N(*)c1cccc2c1c1ccccc1n2-c1ccccc1] | 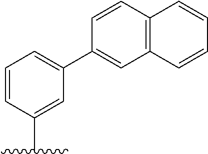 [SMILES: *c1cccc(-c2ccc3ccccc3c2)c1] |
| H14 | 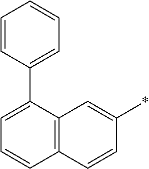 [SMILES: *c1ccc2cccc(-c3ccccc3)c2c1] | 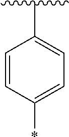 [SMILES: *c1ccc(*)cc1] | 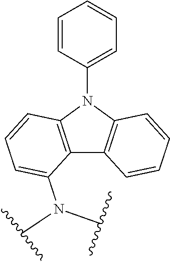 [SMILES: *N(*)c1cccc2c1c1ccccc1n2-c1ccccc1] | 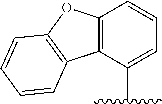 [SMILES: *c1cccc2oc3ccccc3c12] |
| H15 | 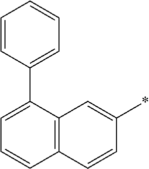 [SMILES: *c1ccc2cccc(-c3ccccc3)c2c1] | 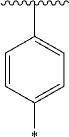 [SMILES: *c1ccc(*)cc1] | 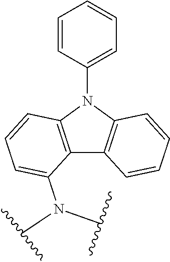 [SMILES: *N(*)c1cccc2c1c1ccccc1n2-c1ccccc1] | 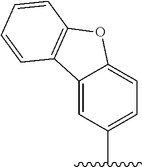 [SMILES: *c1ccc2oc3ccccc3c2c1] |
| H16 | 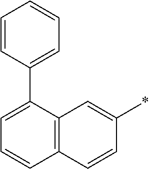 [SMILES: *c1ccc2cccc(-c3ccccc3)c2c1] | 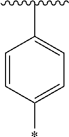 [SMILES: *c1ccc(*)cc1] | 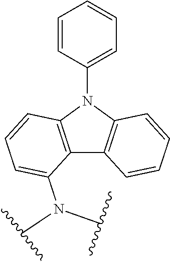 [SMILES: *N(*)c1cccc2c1c1ccccc1n2-c1ccccc1] | 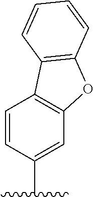 [SMILES: *c1ccc2c(c1)oc1ccccc12] |
| H17 | 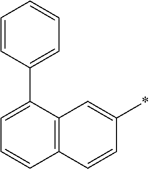 [SMILES: *c1ccc2cccc(-c3ccccc3)c2c1] | 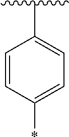 [SMILES: *c1ccc(*)cc1] | 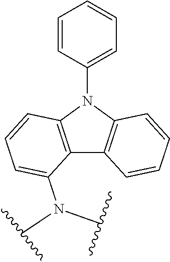 [SMILES: *N(*)c1cccc2c1c1ccccc1n2-c1ccccc1] | 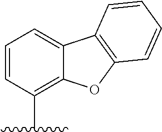 [SMILES: *c1cccc2c1oc1ccccc12] |
| H18 | 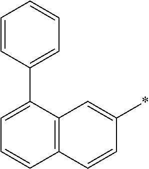 [SMILES: *c1ccc2cccc(-c3ccccc3)c2c1] | 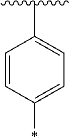 [SMILES: *c1ccc(*)cc1] | 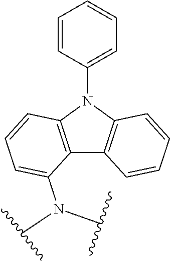 [SMILES: *N(*)c1cccc2c1c1ccccc1n2-c1ccccc1] | 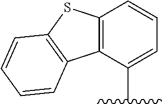 [SMILES: *c1cccc2sc3ccccc3c12] |
| H19 | 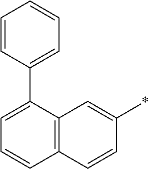 [SMILES: *c1ccc2cccc(-c3ccccc3)c2c1] | 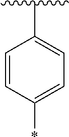 [SMILES: *c1ccc(*)cc1] | 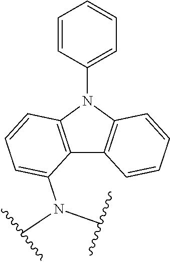 [SMILES: *N(*)c1cccc2c1c1ccccc1n2-c1ccccc1] | 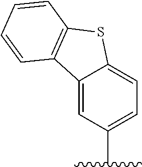 [SMILES: *c1ccc2sc3ccccc3c2c1] |
| H20 | 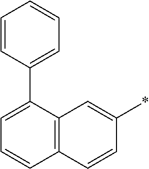 [SMILES: *c1ccc2cccc(-c3ccccc3)c2c1] | 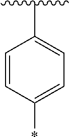 [SMILES: *c1ccc(*)cc1] | 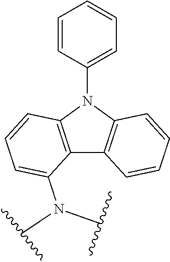 [SMILES: *N(*)c1cccc2c1c1ccccc1n2-c1ccccc1] | 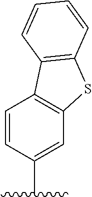 [SMILES: *c1ccc2c(c1)sc1ccccc12] |
| H21 | 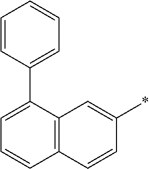 [SMILES: *c1ccc2cccc(-c3ccccc3)c2c1] | 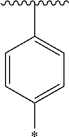 [SMILES: *c1ccc(*)cc1] | 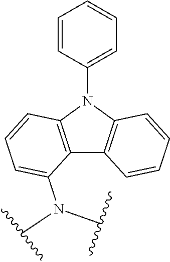 [SMILES: *N(*)c1cccc2c1c1ccccc1n2-c1ccccc1] | 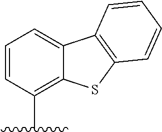 [SMILES: *c1cccc2c1sc1ccccc12] |
| H22 | 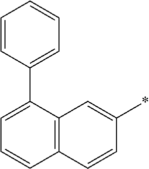 [SMILES: *c1ccc2cccc(-c3ccccc3)c2c1] | 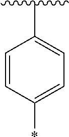 [SMILES: *c1ccc(*)cc1] | 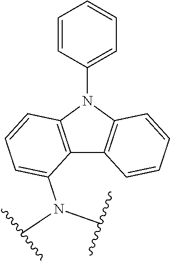 [SMILES: *N(*)c1cccc2c1c1ccccc1n2-c1ccccc1] | 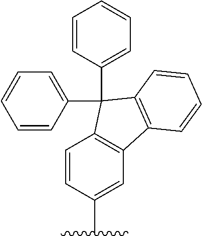 [SMILES: *c1ccc2c(c1)-c1ccccc1C2(c1ccccc1)c1ccccc1] |
| H23 | 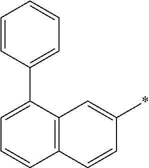 [SMILES: *c1ccc2cccc(-c3ccccc3)c2c1] | 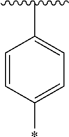 [SMILES: *c1ccc(*)cc1] | 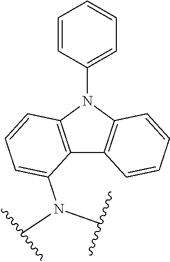 [SMILES: *N(*)c1cccc2c1c1ccccc1n2-c1ccccc1] | 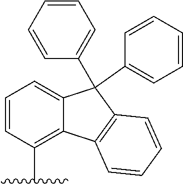 [SMILES: *c1cccc2c1-c1ccccc1C2(c1ccccc1)c1ccccc1] |
| H24 | 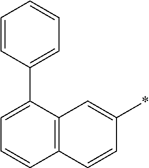 [SMILES: *c1ccc2cccc(-c3ccccc3)c2c1] | 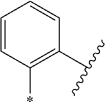 [SMILES: *c1ccccc1*] | 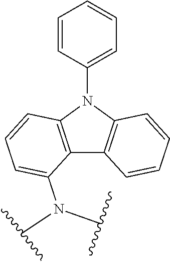 [SMILES: *N(*)c1cccc2c1c1ccccc1n2-c1ccccc1] | 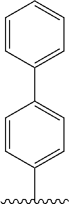 [SMILES: *c1ccc(-c2ccccc2)cc1] |
| H25 | 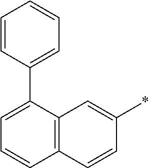 [SMILES: *c1ccc2cccc(-c3ccccc3)c2c1] | 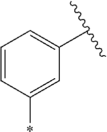 [SMILES: *c1cccc(*)c1] | 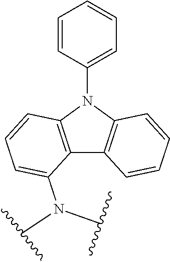 [SMILES: *N(*)c1cccc2c1c1ccccc1n2-c1ccccc1] | 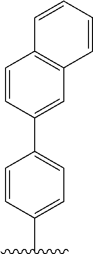 [SMILES: *c1ccc(-c2ccc3ccccc3c2)cc1] |
| H26 | 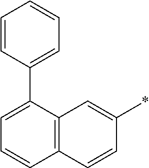 [SMILES: *c1ccc2cccc(-c3ccccc3)c2c1] | 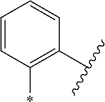 [SMILES: *c1ccccc1*] | 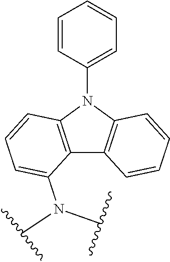 [SMILES: *N(*)c1cccc2c1c1ccccc1n2-c1ccccc1] | 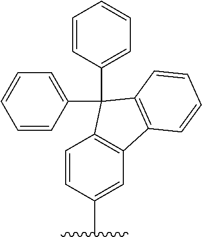 [SMILES: *c1ccc2c(c1)-c1ccccc1C2(c1ccccc1)c1ccccc1] |
| H27 | 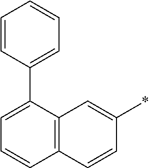 [SMILES: *c1ccc2cccc(-c3ccccc3)c2c1] | 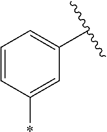 [SMILES: *c1cccc(*)c1] | 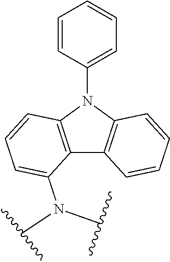 [SMILES: *N(*)c1cccc2c1c1ccccc1n2-c1ccccc1] | 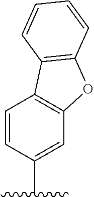 [SMILES: *c1ccc2c(c1)oc1ccccc12] |
| H28 | 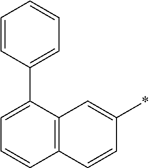 [SMILES: *c1ccc2cccc(-c3ccccc3)c2c1] | 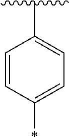 [SMILES: *c1ccc(*)cc1] | 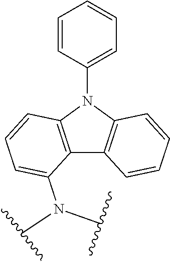 [SMILES: *N(*)c1cccc2c1c1ccccc1n2-c1ccccc1] | 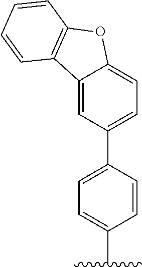 [SMILES: *c1ccc(-c2ccc3oc4ccccc4c3c2)cc1] |
| H29 | 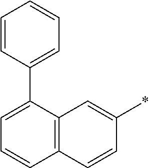 [SMILES: *c1ccc2cccc(-c3ccccc3)c2c1] | 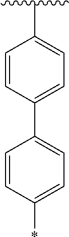 [SMILES: *c1ccc(-c2ccc(*)cc2)cc1] | 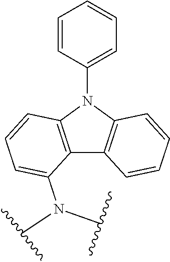 [SMILES: *N(*)c1cccc2c1c1ccccc1n2-c1ccccc1] | 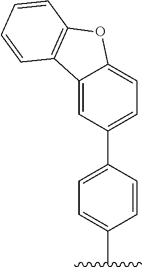 [SMILES: *c1ccc(-c2ccc3oc4ccccc4c3c2)cc1] |
| H30 | 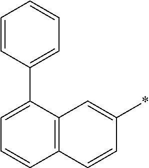 [SMILES: *c1ccc2cccc(-c3ccccc3)c2c1] | 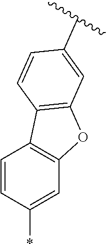 [SMILES: *c1ccc2c(c1)oc1cc(*)ccc12] | 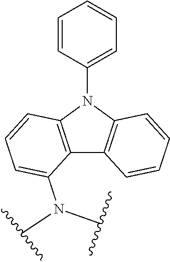 [SMILES: *N(*)c1cccc2c1c1ccccc1n2-c1ccccc1] | 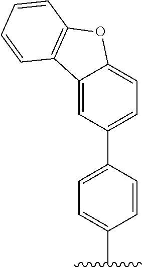 [SMILES: *c1ccc(-c2ccc3oc4ccccc4c3c2)cc1] |
| H31 | 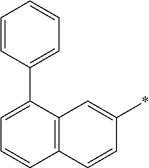 [SMILES: *c1ccc2cccc(-c3ccccc3)c2c1] | 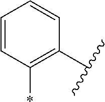 [SMILES: *c1ccccc1*] | 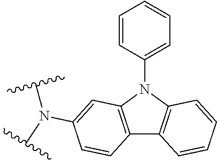 [SMILES: *N(*)c1ccc2c3ccccc3n(-c3ccccc3)c2c1] | 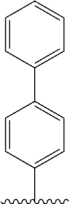 [SMILES: *c1ccc(-c2ccccc2)cc1] |
| H32 | 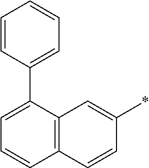 [SMILES: *c1ccc2cccc(-c3ccccc3)c2c1] | 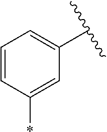 [SMILES: *c1cccc(*)c1] | 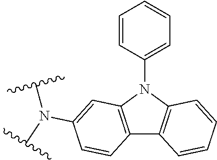 [SMILES: *N(*)c1ccc2c3ccccc3n(-c3ccccc3)c2c1] | 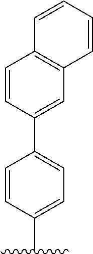 [SMILES: *c1ccc(-c2ccc3ccccc3c2)cc1] |
| H33 | 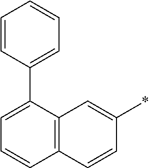 [SMILES: *c1ccc2cccc(-c3ccccc3)c2c1] | 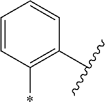 [SMILES: *c1ccccc1*] | 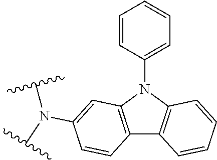 [SMILES: *N(*)c1ccc2c3ccccc3n(-c3ccccc3)c2c1] | 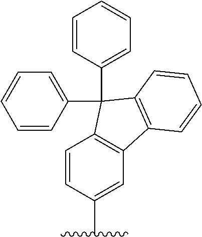 [SMILES: *c1ccc2c(c1)-c1ccccc1C2(c1ccccc1)c1ccccc1] |
| H34 | 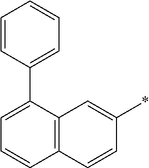 [SMILES: *c1ccc2cccc(-c3ccccc3)c2c1] | 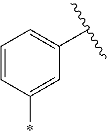 [SMILES: *c1cccc(*)c1] | 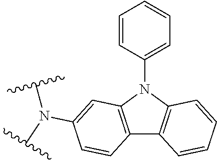 [SMILES: *N(*)c1ccc2c3ccccc3n(-c3ccccc3)c2c1] | 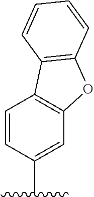 [SMILES: *c1ccc2c(c1)oc1ccccc12] |
| H35 | 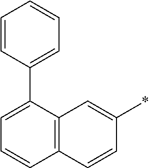 [SMILES: *c1ccc2cccc(-c3ccccc3)c2c1] | 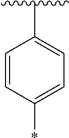 [SMILES: *c1ccc(*)cc1] | 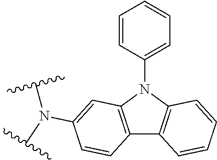 [SMILES: *N(*)c1ccc2c3ccccc3n(-c3ccccc3)c2c1] | 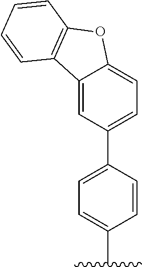 [SMILES: *c1ccc(-c2ccc3oc4ccccc4c3c2)cc1] |
| H36 | 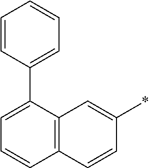 [SMILES: *c1ccc2cccc(-c3ccccc3)c2c1] | 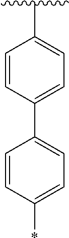 [SMILES: *c1ccc(-c2ccc(*)cc2)cc1] | 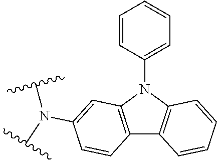 [SMILES: *N(*)c1ccc2c3ccccc3n(-c3ccccc3)c2c1] | 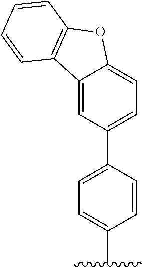 [SMILES: *c1ccc(-c2ccc3oc4ccccc4c3c2)cc1] |
| H37 | 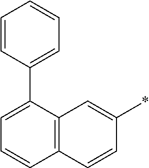 [SMILES: *c1ccc2cccc(-c3ccccc3)c2c1] | 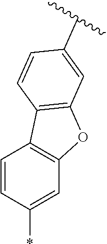 [SMILES: *c1ccc2c(c1)oc1cc(*)ccc12] | 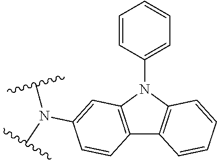 [SMILES: *N(*)c1ccc2c3ccccc3n(-c3ccccc3)c2c1] | 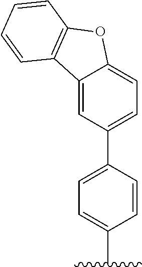 [SMILES: *c1ccc(-c2ccc3oc4ccccc4c3c2)cc1] |
| I1 | 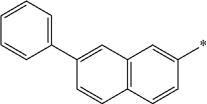 [SMILES: *c1ccc2ccc(-c3ccccc3)cc2c1] | 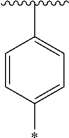 [SMILES: *c1ccc(*)cc1] | 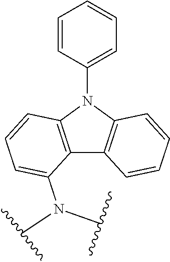 [SMILES: *N(*)c1cccc2c1c1ccccc1n2-c1ccccc1] | 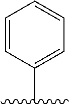 [SMILES: *c1ccccc1] |
| I2 | 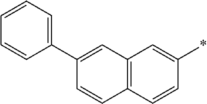 [SMILES: *c1ccc2ccc(-c3ccccc3)cc2c1] | 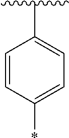 [SMILES: *c1ccc(*)cc1] | 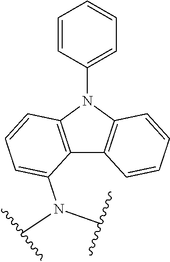 [SMILES: *N(*)c1cccc2c1c1ccccc1n2-c1ccccc1] | 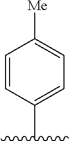 [SMILES: *c1ccc(C)cc1] |
| I3 | 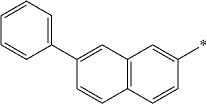 [SMILES: *c1ccc2ccc(-c3ccccc3)cc2c1] | 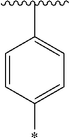 [SMILES: *c1ccc(*)cc1] | 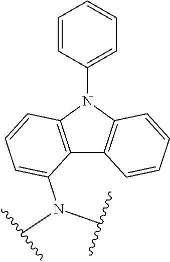 [SMILES: *N(*)c1cccc2c1c1ccccc1n2-c1ccccc1] | 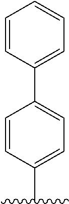 [SMILES: *c1ccc(-c2ccccc2)cc1] |
| I4 | 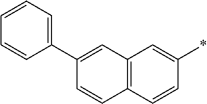 [SMILES: *c1ccc2ccc(-c3ccccc3)cc2c1] | 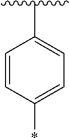 [SMILES: *c1ccc(*)cc1] | 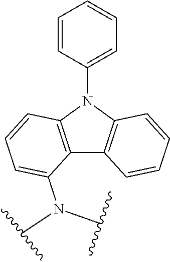 [SMILES: *N(*)c1cccc2c1c1ccccc1n2-c1ccccc1] | 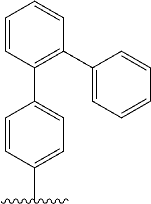 [SMILES: *c1ccc(-c2ccccc2-c2ccccc2)cc1] |
| I5 | 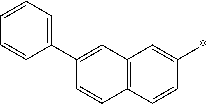 [SMILES: *c1ccc2ccc(-c3ccccc3)cc2c1] | 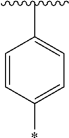 [SMILES: *c1ccc(*)cc1] | 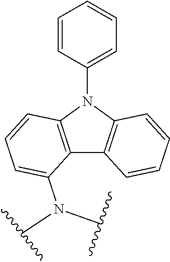 [SMILES: *N(*)c1cccc2c1c1ccccc1n2-c1ccccc1] | 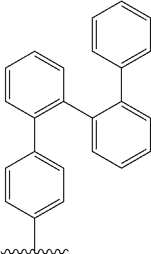 [SMILES: *c1ccc(-c2ccccc2-c2ccccc2-c2ccccc2)cc1] |
| I6 | 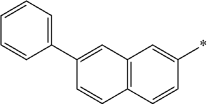 [SMILES: *c1ccc2ccc(-c3ccccc3)cc2c1] | 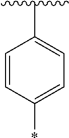 [SMILES: *c1ccc(*)cc1] | 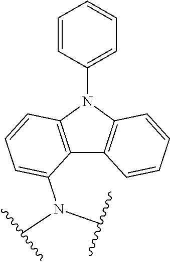 [SMILES: *N(*)c1cccc2c1c1ccccc1n2-c1ccccc1] | 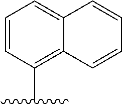 [SMILES: *c1cccc2ccccc12] |
| I7 | 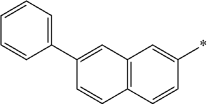 [SMILES: *c1ccc2ccc(-c3ccccc3)cc2c1] | 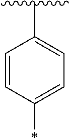 [SMILES: *c1ccc(*)cc1] | 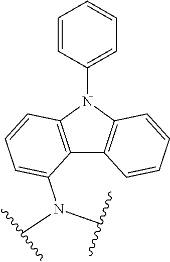 [SMILES: *N(*)c1cccc2c1c1ccccc1n2-c1ccccc1] | 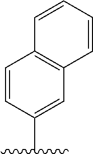 [SMILES: *c1ccc2ccccc2c1] |
| I8 | 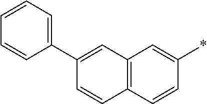 [SMILES: *c1ccc2ccc(-c3ccccc3)cc2c1] | 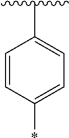 [SMILES: *c1ccc(*)cc1] | 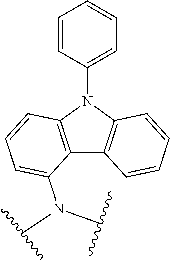 [SMILES: *N(*)c1cccc2c1c1ccccc1n2-c1ccccc1] | 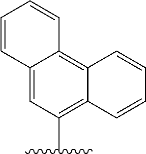 [SMILES: *c1cc2ccccc2c2ccccc12] |
| I9 | 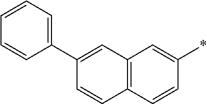 [SMILES: *c1ccc2ccc(-c3ccccc3)cc2c1] | 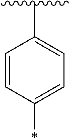 [SMILES: *c1ccc(*)cc1] | 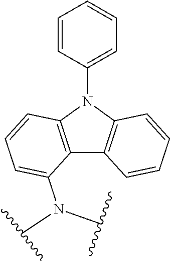 [SMILES: *N(*)c1cccc2c1c1ccccc1n2-c1ccccc1] | 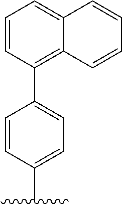 [SMILES: *c1ccc(-c2cccc3ccccc23)cc1] |
| I10 | 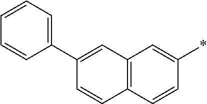 [SMILES: *c1ccc2ccc(-c3ccccc3)cc2c1] | 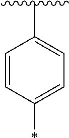 [SMILES: *c1ccc(*)cc1] | 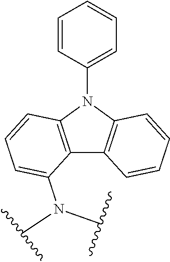 [SMILES: *N(*)c1cccc2c1c1ccccc1n2-c1ccccc1] | 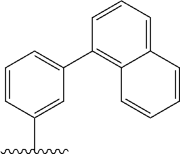 [SMILES: *c1cccc(-c2cccc3ccccc23)c1] |
| I11 | 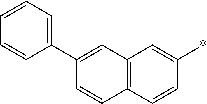 [SMILES: *c1ccc2ccc(-c3ccccc3)cc2c1] | 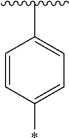 [SMILES: *c1ccc(*)cc1] | 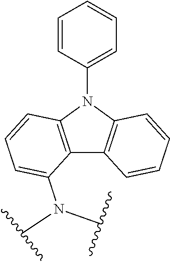 [SMILES: *N(*)c1cccc2c1c1ccccc1n2-c1ccccc1] | 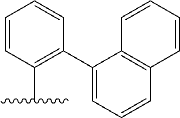 [SMILES: *c1ccccc1-c1cccc2ccccc12] |
| I12 | 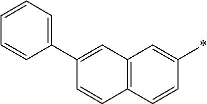 [SMILES: *c1ccc2ccc(-c3ccccc3)cc2c1] | 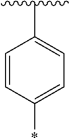 [SMILES: *c1ccc(*)cc1] | 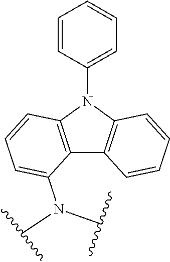 [SMILES: *N(*)c1cccc2c1c1ccccc1n2-c1ccccc1] | 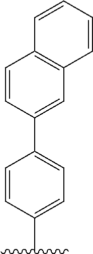 [SMILES: *c1ccc(-c2ccc3ccccc3c2)cc1] |
| I13 | 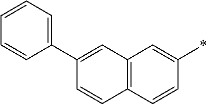 [SMILES: *c1ccc2ccc(-c3ccccc3)cc2c1] | 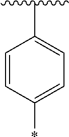 [SMILES: *c1ccc(*)cc1] | 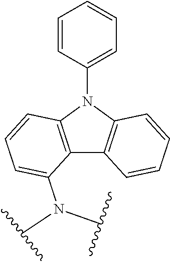 [SMILES: *N(*)c1cccc2c1c1ccccc1n2-c1ccccc1] | 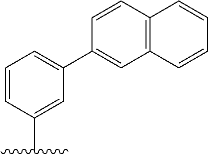 [SMILES: *c1cccc(-c2ccc3ccccc3c2)c1] |
| I14 | 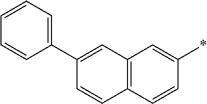 [SMILES: *c1ccc2ccc(-c3ccccc3)cc2c1] | 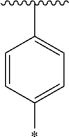 [SMILES: *c1ccc(*)cc1] | 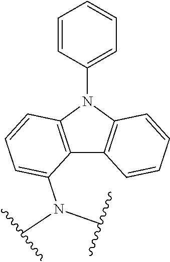 [SMILES: *N(*)c1cccc2c1c1ccccc1n2-c1ccccc1] | 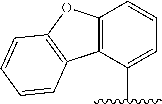 [SMILES: *c1cccc2oc3ccccc3c12] |
| I15 | 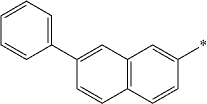 [SMILES: *c1ccc2ccc(-c3ccccc3)cc2c1] | 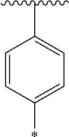 [SMILES: *c1ccc(*)cc1] | 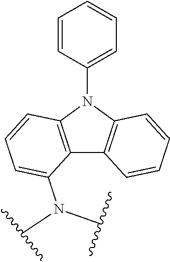 [SMILES: *N(*)c1cccc2c1c1ccccc1n2-c1ccccc1] | 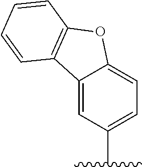 [SMILES: *c1ccc2oc3ccccc3c2c1] |
| I16 | 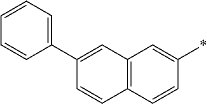 [SMILES: *c1ccc2ccc(-c3ccccc3)cc2c1] | 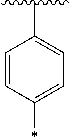 [SMILES: *c1ccc(*)cc1] | 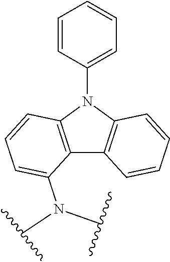 [SMILES: *N(*)c1cccc2c1c1ccccc1n2-c1ccccc1] | 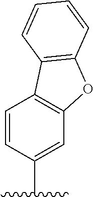 [SMILES: *c1ccc2c(c1)oc1ccccc12] |
| I17 | 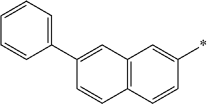 [SMILES: *c1ccc2ccc(-c3ccccc3)cc2c1] | 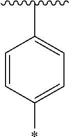 [SMILES: *c1ccc(*)cc1] | 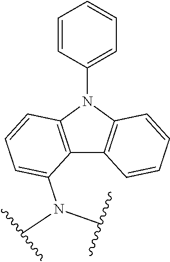 [SMILES: *N(*)c1cccc2c1c1ccccc1n2-c1ccccc1] | 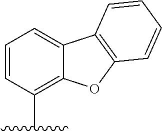 [SMILES: *c1cccc2c1oc1ccccc12] |
| I18 | 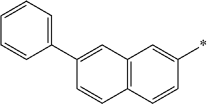 [SMILES: *c1ccc2ccc(-c3ccccc3)cc2c1] | 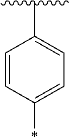 [SMILES: *c1ccc(*)cc1] | 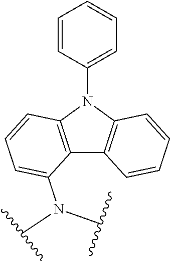 [SMILES: *N(*)c1cccc2c1c1ccccc1n2-c1ccccc1] | 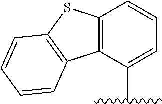 [SMILES: *c1cccc2sc3ccccc3c12] |
| I19 | 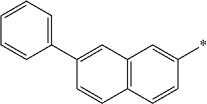 [SMILES: *c1ccc2ccc(-c3ccccc3)cc2c1] | 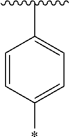 [SMILES: *c1ccc(*)cc1] | 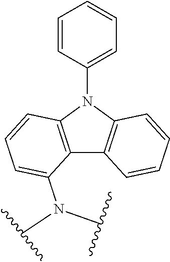 [SMILES: *N(*)c1cccc2c1c1ccccc1n2-c1ccccc1] | 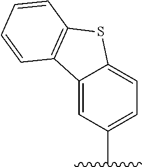 [SMILES: *c1ccc2sc3ccccc3c2c1] |
| I20 | 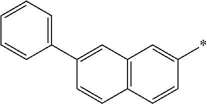 [SMILES: *c1ccc2ccc(-c3ccccc3)cc2c1] | 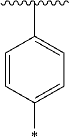 [SMILES: *c1ccc(*)cc1] | 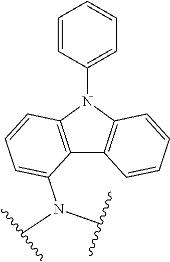 [SMILES: *N(*)c1cccc2c1c1ccccc1n2-c1ccccc1] | 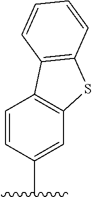 [SMILES: *c1ccc2c(c1)sc1ccccc12] |
| I21 | 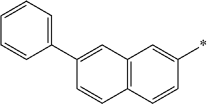 [SMILES: *c1ccc2ccc(-c3ccccc3)cc2c1] | 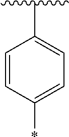 [SMILES: *c1ccc(*)cc1] | 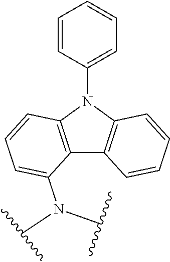 [SMILES: *N(*)c1cccc2c1c1ccccc1n2-c1ccccc1] | 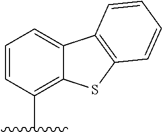 [SMILES: *c1cccc2c1sc1ccccc12] |
| I22 | 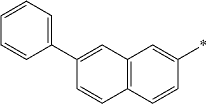 [SMILES: *c1ccc2ccc(-c3ccccc3)cc2c1] | 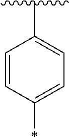 [SMILES: *c1ccc(*)cc1] | 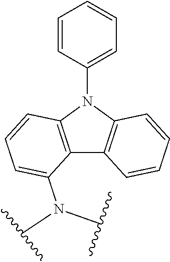 [SMILES: *N(*)c1cccc2c1c1ccccc1n2-c1ccccc1] | 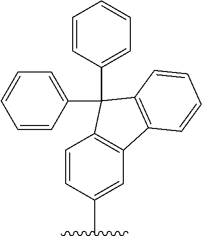 [SMILES: *c1ccc2c(c1)-c1ccccc1C2(c1ccccc1)c1ccccc1] |
| I23 | 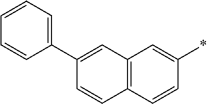 [SMILES: *c1ccc2ccc(-c3ccccc3)cc2c1] | 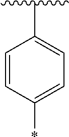 [SMILES: *c1ccc(*)cc1] | 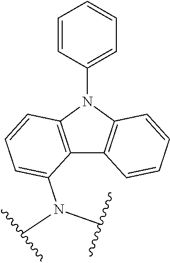 [SMILES: *N(*)c1cccc2c1c1ccccc1n2-c1ccccc1] | 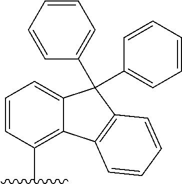 [SMILES: *c1cccc2c1-c1ccccc1C2(c1ccccc1)c1ccccc1] |
| I24 | 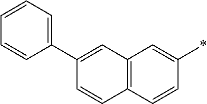 [SMILES: *c1ccc2ccc(-c3ccccc3)cc2c1] | 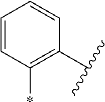 [SMILES: *c1ccccc1*] | 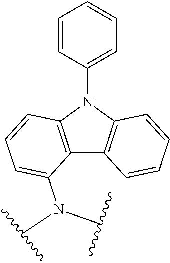 [SMILES: *N(*)c1cccc2c1c1ccccc1n2-c1ccccc1] | 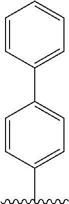 [SMILES: *c1ccc(-c2ccccc2)cc1] |
| I25 | 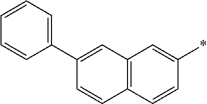 [SMILES: *c1ccc2ccc(-c3ccccc3)cc2c1] | 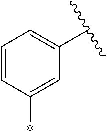 [SMILES: *c1cccc(*)c1] | 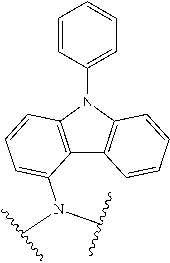 [SMILES: *N(*)c1cccc2c1c1ccccc1n2-c1ccccc1] | 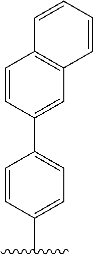 [SMILES: *c1ccc(-c2ccc3ccccc3c2)cc1] |
| I26 | 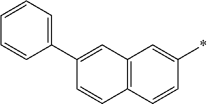 [SMILES: *c1ccc2ccc(-c3ccccc3)cc2c1] | 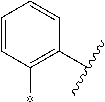 [SMILES: *c1ccccc1*] | 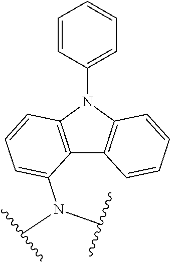 [SMILES: *N(*)c1cccc2c1c1ccccc1n2-c1ccccc1] | 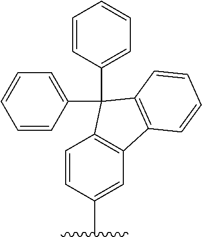 [SMILES: *c1ccc2c(c1)-c1ccccc1C2(c1ccccc1)c1ccccc1] |
| I27 | 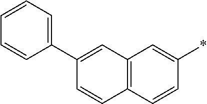 [SMILES: *c1ccc2ccc(-c3ccccc3)cc2c1] | 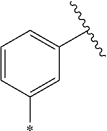 [SMILES: *c1cccc(*)c1] | 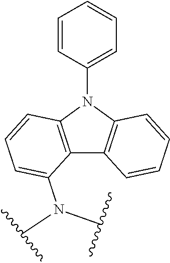 [SMILES: *N(*)c1cccc2c1c1ccccc1n2-c1ccccc1] | 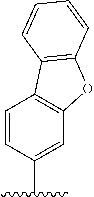 [SMILES: *c1ccc2c(c1)oc1ccccc12] |
| I28 | 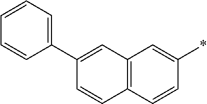 [SMILES: *c1ccc2ccc(-c3ccccc3)cc2c1] | 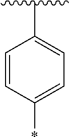 [SMILES: *c1ccc(*)cc1] | 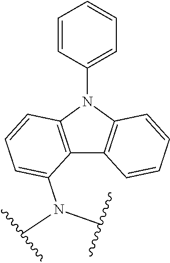 [SMILES: *N(*)c1cccc2c1c1ccccc1n2-c1ccccc1] | 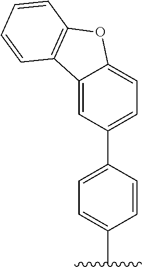 [SMILES: *c1ccc(-c2ccc3oc4ccccc4c3c2)cc1] |
| I29 | 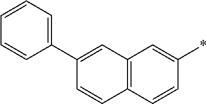 [SMILES: *c1ccc2ccc(-c3ccccc3)cc2c1] | 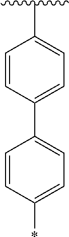 [SMILES: *c1ccc(-c2ccc(*)cc2)cc1] | 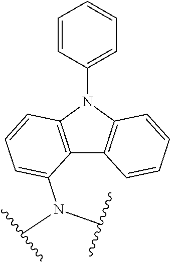 [SMILES: *N(*)c1cccc2c1c1ccccc1n2-c1ccccc1] | 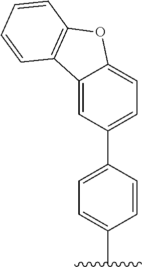 [SMILES: *c1ccc(-c2ccc3oc4ccccc4c3c2)cc1] |
| I30 | 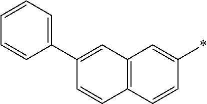 [SMILES: *c1ccc2ccc(-c3ccccc3)cc2c1] | 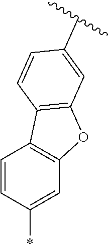 [SMILES: *c1ccc2c(c1)oc1cc(*)ccc12] | 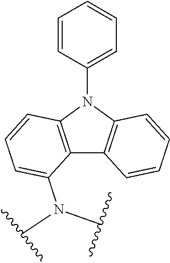 [SMILES: *N(*)c1cccc2c1c1ccccc1n2-c1ccccc1] | 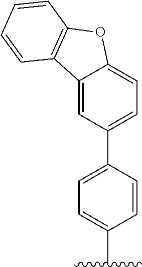 [SMILES: *c1ccc(-c2ccc3oc4ccccc4c3c2)cc1] |
| I31 | 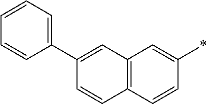 [SMILES: *c1ccc2ccc(-c3ccccc3)cc2c1] | 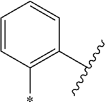 [SMILES: *c1ccccc1*] | 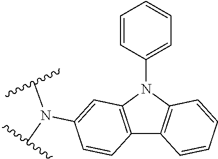 [SMILES: *N(*)c1ccc2c3ccccc3n(-c3ccccc3)c2c1] | 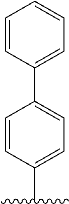 [SMILES: *c1ccc(-c2ccccc2)cc1] |
| I32 | 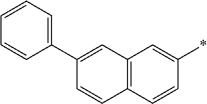 [SMILES: *c1ccc2ccc(-c3ccccc3)cc2c1] | 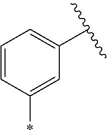 [SMILES: *c1cccc(*)c1] | 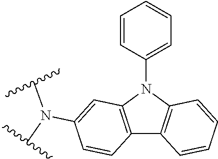 [SMILES: *N(*)c1ccc2c3ccccc3n(-c3ccccc3)c2c1] | 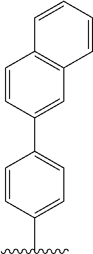 [SMILES: *c1ccc(-c2ccc3ccccc3c2)cc1] |
| I33 | 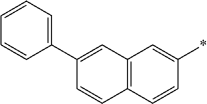 [SMILES: *c1ccc2ccc(-c3ccccc3)cc2c1] | 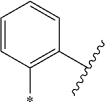 [SMILES: *c1ccccc1*] | 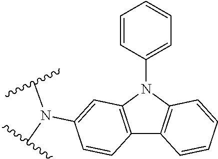 [SMILES: *N(*)c1ccc2c3ccccc3n(-c3ccccc3)c2c1] | 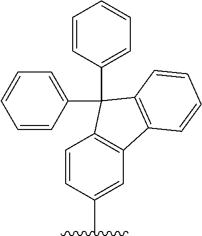 [SMILES: *c1ccc2c(c1)-c1ccccc1C2(c1ccccc1)c1ccccc1] |
| I34 | 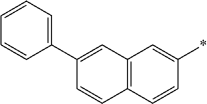 [SMILES: *c1ccc2ccc(-c3ccccc3)cc2c1] | 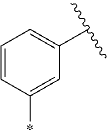 [SMILES: *c1cccc(*)c1] | 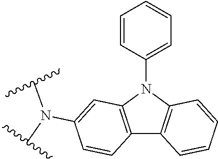 [SMILES: *N(*)c1ccc2c3ccccc3n(-c3ccccc3)c2c1] | 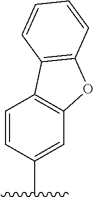 [SMILES: *c1ccc2c(c1)oc1ccccc12] |
| I35 | 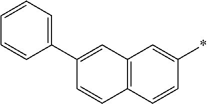 [SMILES: *c1ccc2ccc(-c3ccccc3)cc2c1] | 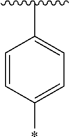 [SMILES: *c1ccc(*)cc1] | 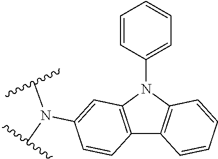 [SMILES: *N(*)c1ccc2c3ccccc3n(-c3ccccc3)c2c1] | 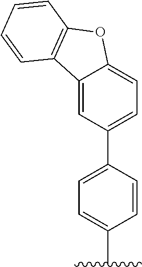 [SMILES: *c1ccc(-c2ccc3oc4ccccc4c3c2)cc1] |
| I36 | 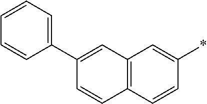 [SMILES: *c1ccc2ccc(-c3ccccc3)cc2c1] | 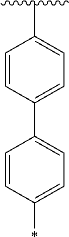 [SMILES: *c1ccc(-c2ccc(*)cc2)cc1] | 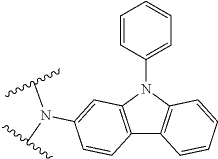 [SMILES: *N(*)c1ccc2c3ccccc3n(-c3ccccc3)c2c1] | 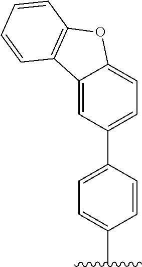 [SMILES: *c1ccc(-c2ccc3oc4ccccc4c3c2)cc1] |
| I37 | 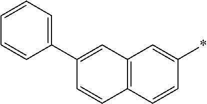 [SMILES: *c1ccc2ccc(-c3ccccc3)cc2c1] | 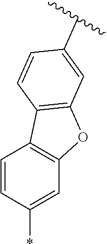 [SMILES: *c1ccc2c(c1)oc1cc(*)ccc12] | 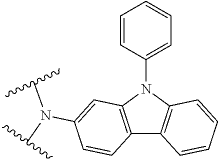 [SMILES: *N(*)c1ccc2c3ccccc3n(-c3ccccc3)c2c1] | 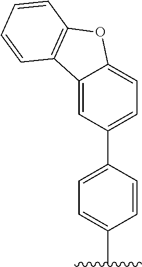 [SMILES: *c1ccc(-c2ccc3oc4ccccc4c3c2)cc1] |
| J1 | 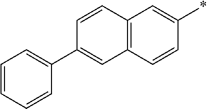 [SMILES: *c1ccc2cc(-c3ccccc3)ccc2c1] | 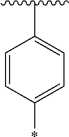 [SMILES: *c1ccc(*)cc1] | 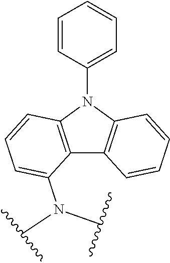 [SMILES: *N(*)c1cccc2c1c1ccccc1n2-c1ccccc1] | 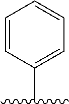 [SMILES: *c1ccccc1] |
| J2 | 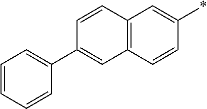 [SMILES: *c1ccc2cc(-c3ccccc3)ccc2c1] | 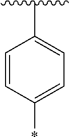 [SMILES: *c1ccc(*)cc1] | 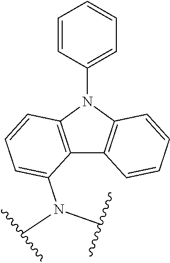 [SMILES: *N(*)c1cccc2c1c1ccccc1n2-c1ccccc1] | 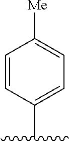 [SMILES: *c1ccc(C)cc1] |
| J3 | 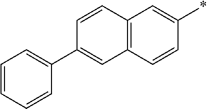 [SMILES: *c1ccc2cc(-c3ccccc3)ccc2c1] | 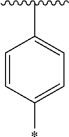 [SMILES: *c1ccc(*)cc1] | 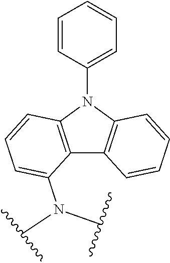 [SMILES: *N(*)c1cccc2c1c1ccccc1n2-c1ccccc1] | 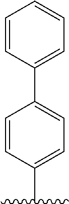 [SMILES: *c1ccc(-c2ccccc2)cc1] |
| J4 | 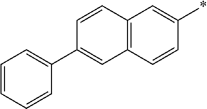 [SMILES: *c1ccc2cc(-c3ccccc3)ccc2c1] | 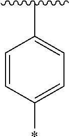 [SMILES: *c1ccc(*)cc1] | 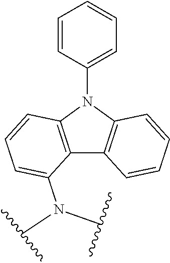 [SMILES: *N(*)c1cccc2c1c1ccccc1n2-c1ccccc1] | 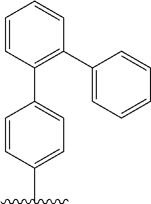 [SMILES: *c1ccc(-c2ccccc2-c2ccccc2)cc1] |
| J5 | 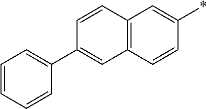 [SMILES: *c1ccc2cc(-c3ccccc3)ccc2c1] | 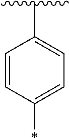 [SMILES: *c1ccc(*)cc1] | 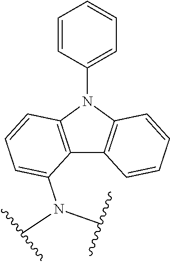 [SMILES: *N(*)c1cccc2c1c1ccccc1n2-c1ccccc1] | 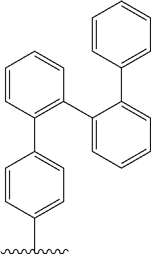 [SMILES: *c1ccc(-c2ccccc2-c2ccccc2-c2ccccc2)cc1] |
| J6 | 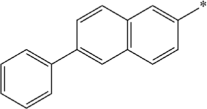 [SMILES: *c1ccc2cc(-c3ccccc3)ccc2c1] | 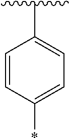 [SMILES: *c1ccc(*)cc1] | 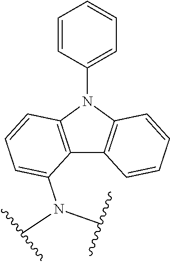 [SMILES: *N(*)c1cccc2c1c1ccccc1n2-c1ccccc1] | 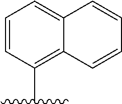 [SMILES: *c1cccc2ccccc12] |
| J7 | 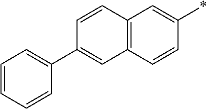 [SMILES: *c1ccc2cc(-c3ccccc3)ccc2c1] | 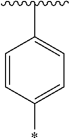 [SMILES: *c1ccc(*)cc1] | 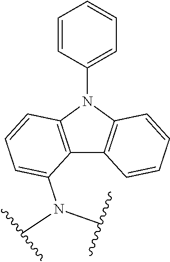 [SMILES: *N(*)c1cccc2c1c1ccccc1n2-c1ccccc1] | 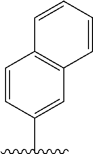 [SMILES: *c1ccc2ccccc2c1] |
| J8 | 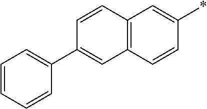 [SMILES: *c1ccc2cc(-c3ccccc3)ccc2c1] | 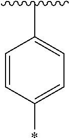 [SMILES: *c1ccc(*)cc1] | 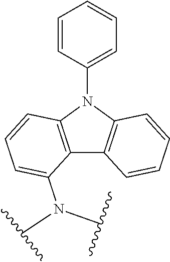 [SMILES: *N(*)c1cccc2c1c1ccccc1n2-c1ccccc1] | 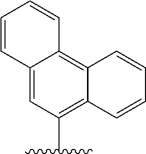 [SMILES: *c1cc2ccccc2c2ccccc12] |
| J9 | 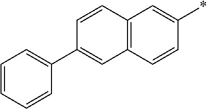 [SMILES: *c1ccc2cc(-c3ccccc3)ccc2c1] | 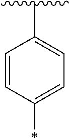 [SMILES: *c1ccc(*)cc1] | 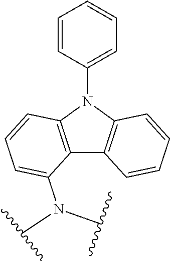 [SMILES: *N(*)c1cccc2c1c1ccccc1n2-c1ccccc1] | 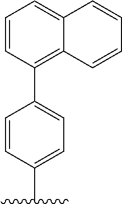 [SMILES: *c1ccc(-c2cccc3ccccc23)cc1] |
| J10 | 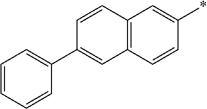 [SMILES: *c1ccc2cc(-c3ccccc3)ccc2c1] | 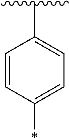 [SMILES: *c1ccc(*)cc1] | 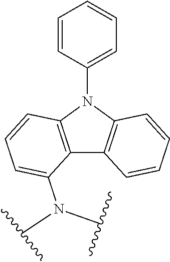 [SMILES: *N(*)c1cccc2c1c1ccccc1n2-c1ccccc1] | 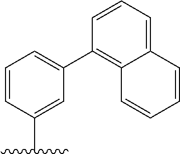 [SMILES: *c1cccc(-c2cccc3ccccc23)c1] |
| J11 | 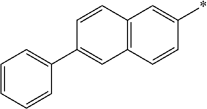 [SMILES: *c1ccc2cc(-c3ccccc3)ccc2c1] | 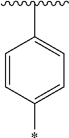 [SMILES: *c1ccc(*)cc1] | 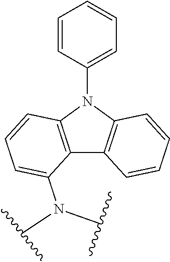 [SMILES: *N(*)c1cccc2c1c1ccccc1n2-c1ccccc1] | 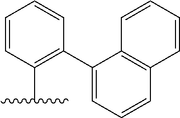 [SMILES: *c1ccccc1-c1cccc2ccccc12] |
| J12 | 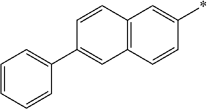 [SMILES: *c1ccc2cc(-c3ccccc3)ccc2c1] | 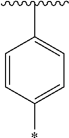 [SMILES: *c1ccc(*)cc1] | 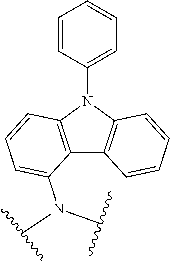 [SMILES: *N(*)c1cccc2c1c1ccccc1n2-c1ccccc1] | 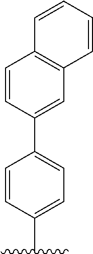 [SMILES: *c1ccc(-c2ccc3ccccc3c2)cc1] |
| J13 | 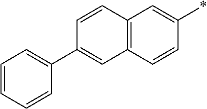 [SMILES: *c1ccc2cc(-c3ccccc3)ccc2c1] | 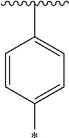 [SMILES: *c1ccc(*)cc1] | 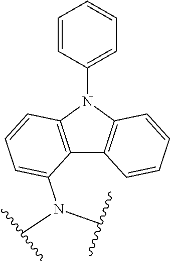 [SMILES: *N(*)c1cccc2c1c1ccccc1n2-c1ccccc1] | 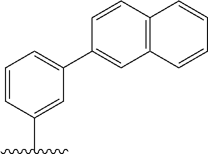 [SMILES: *c1cccc(-c2ccc3ccccc3c2)c1] |
| J14 | 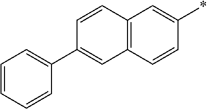 [SMILES: *c1ccc2cc(-c3ccccc3)ccc2c1] | 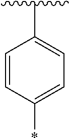 [SMILES: *c1ccc(*)cc1] | 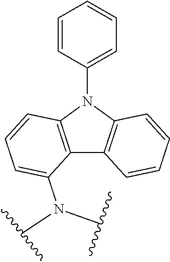 [SMILES: *N(*)c1cccc2c1c1ccccc1n2-c1ccccc1] | 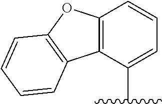 [SMILES: *c1cccc2oc3ccccc3c12] |
| J15 | 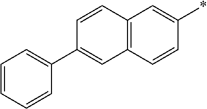 [SMILES: *c1ccc2cc(-c3ccccc3)ccc2c1] | 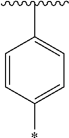 [SMILES: *c1ccc(*)cc1] | 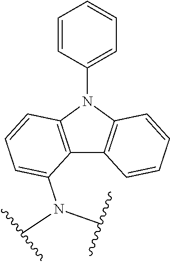 [SMILES: *N(*)c1cccc2c1c1ccccc1n2-c1ccccc1] | 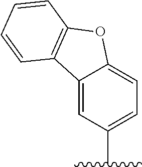 [SMILES: *c1ccc2oc3ccccc3c2c1] |
| J16 | 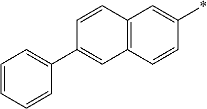 [SMILES: *c1ccc2cc(-c3ccccc3)ccc2c1] | 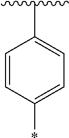 [SMILES: *c1ccc(*)cc1] | 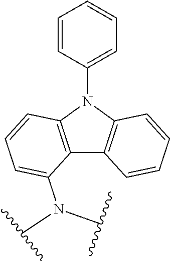 [SMILES: *N(*)c1cccc2c1c1ccccc1n2-c1ccccc1] | 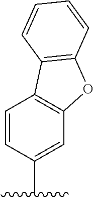 [SMILES: *c1ccc2c(c1)oc1ccccc12] |
| J17 | 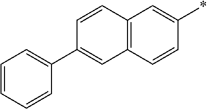 [SMILES: *c1ccc2cc(-c3ccccc3)ccc2c1] | 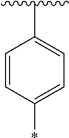 [SMILES: *c1ccc(*)cc1] | 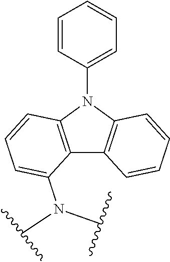 [SMILES: *N(*)c1cccc2c1c1ccccc1n2-c1ccccc1] | 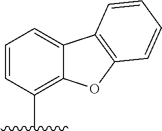 [SMILES: *c1cccc2c1oc1ccccc12] |
| J18 | 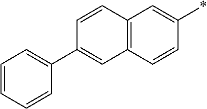 [SMILES: *c1ccc2cc(-c3ccccc3)ccc2c1] | 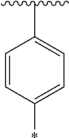 [SMILES: *c1ccc(*)cc1] | 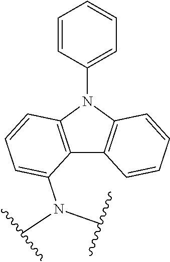 [SMILES: *N(*)c1cccc2c1c1ccccc1n2-c1ccccc1] | 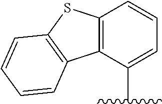 [SMILES: *c1cccc2sc3ccccc3c12] |
| J19 | 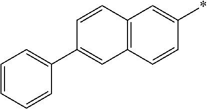 [SMILES: *c1ccc2cc(-c3ccccc3)ccc2c1] | 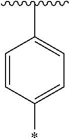 [SMILES: *c1ccc(*)cc1] | 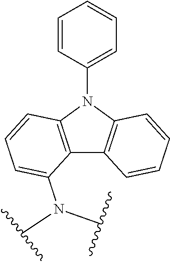 [SMILES: *N(*)c1cccc2c1c1ccccc1n2-c1ccccc1] | 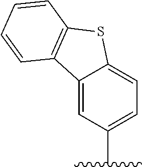 [SMILES: *c1ccc2sc3ccccc3c2c1] |
| J20 | 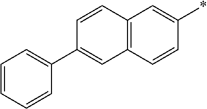 [SMILES: *c1ccc2cc(-c3ccccc3)ccc2c1] | 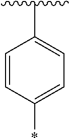 [SMILES: *c1ccc(*)cc1] | 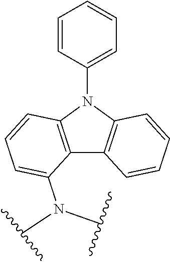 [SMILES: *N(*)c1cccc2c1c1ccccc1n2-c1ccccc1] | 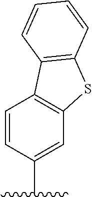 [SMILES: *c1ccc2c(c1)sc1ccccc12] |
| J21 | 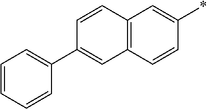 [SMILES: *c1ccc2cc(-c3ccccc3)ccc2c1] | 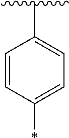 [SMILES: *c1ccc(*)cc1] | 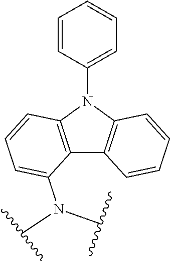 [SMILES: *N(*)c1cccc2c1c1ccccc1n2-c1ccccc1] | 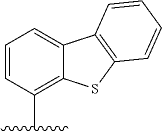 [SMILES: *c1cccc2c1sc1ccccc12] |
| J22 | 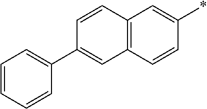 [SMILES: *c1ccc2cc(-c3ccccc3)ccc2c1] | 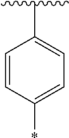 [SMILES: *c1ccc(*)cc1] | 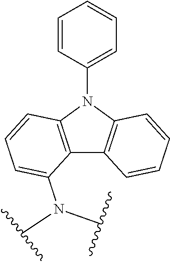 [SMILES: *N(*)c1cccc2c1c1ccccc1n2-c1ccccc1] | 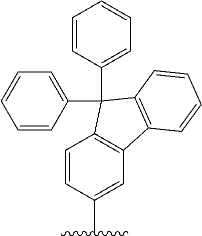 [SMILES: *c1ccc2c(c1)-c1ccccc1C2(c1ccccc1)c1ccccc1] |
| J23 | 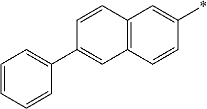 [SMILES: *c1ccc2cc(-c3ccccc3)ccc2c1] | 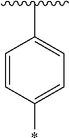 [SMILES: *c1ccc(*)cc1] | 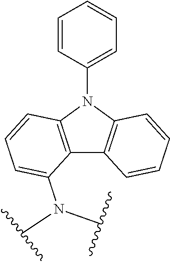 [SMILES: *N(*)c1cccc2c1c1ccccc1n2-c1ccccc1] | 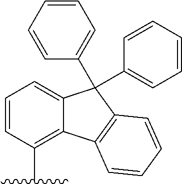 [SMILES: *c1cccc2c1-c1ccccc1C2(c1ccccc1)c1ccccc1] |
| J24 | 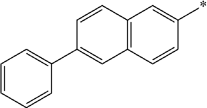 [SMILES: *c1ccc2cc(-c3ccccc3)ccc2c1] | 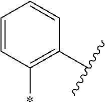 [SMILES: *c1ccccc1*] | 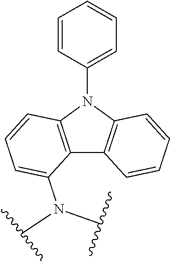 [SMILES: *N(*)c1cccc2c1c1ccccc1n2-c1ccccc1] | 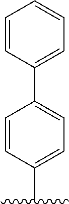 [SMILES: *c1ccc(-c2ccccc2)cc1] |
| J25 | 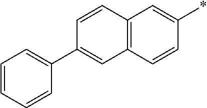 [SMILES: *c1ccc2cc(-c3ccccc3)ccc2c1] | 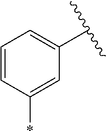 [SMILES: *c1cccc(*)c1] | 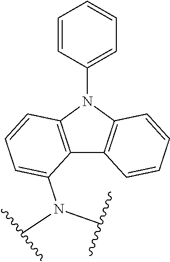 [SMILES: *N(*)c1cccc2c1c1ccccc1n2-c1ccccc1] | 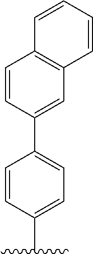 [SMILES: *c1ccc(-c2ccc3ccccc3c2)cc1] |
| J26 | 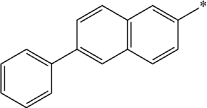 [SMILES: *c1ccc2cc(-c3ccccc3)ccc2c1] | 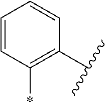 [SMILES: *c1ccccc1*] | 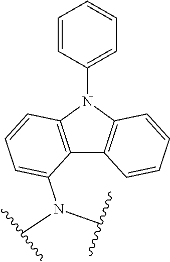 [SMILES: *N(*)c1cccc2c1c1ccccc1n2-c1ccccc1] | 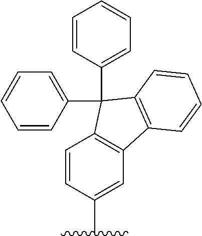 [SMILES: *c1ccc2c(c1)-c1ccccc1C2(c1ccccc1)c1ccccc1] |
| J27 | 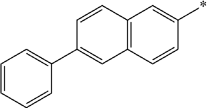 [SMILES: *c1ccc2cc(-c3ccccc3)ccc2c1] | 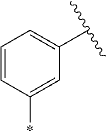 [SMILES: *c1cccc(*)c1] | 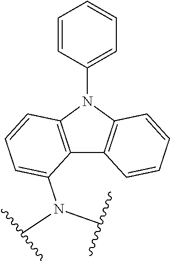 [SMILES: *N(*)c1cccc2c1c1ccccc1n2-c1ccccc1] | 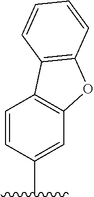 [SMILES: *c1ccc2c(c1)oc1ccccc12] |
| J28 | 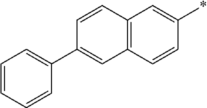 [SMILES: *c1ccc2cc(-c3ccccc3)ccc2c1] | 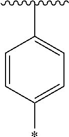 [SMILES: *c1ccc(*)cc1] | 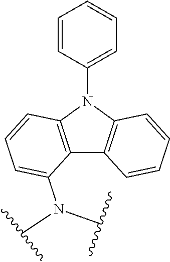 [SMILES: *N(*)c1cccc2c1c1ccccc1n2-c1ccccc1] | 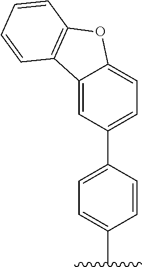 [SMILES: *c1ccc(-c2ccc3oc4ccccc4c3c2)cc1] |
| J29 | 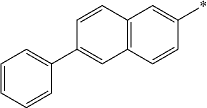 [SMILES: *c1ccc2cc(-c3ccccc3)ccc2c1] | 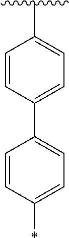 [SMILES: *c1ccc(-c2ccc(*)cc2)cc1] | 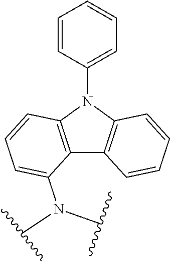 [SMILES: *N(*)c1cccc2c1c1ccccc1n2-c1ccccc1] | 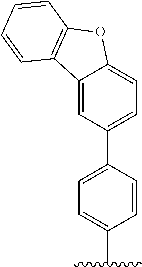 [SMILES: *c1ccc(-c2ccc3oc4ccccc4c3c2)cc1] |
| J30 | 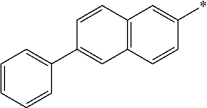 [SMILES: *c1ccc2cc(-c3ccccc3)ccc2c1] | 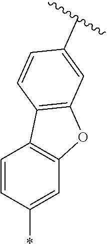 [SMILES: *c1ccc2c(c1)oc1cc(*)ccc12] | 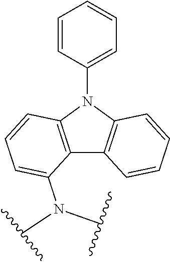 [SMILES: *N(*)c1cccc2c1c1ccccc1n2-c1ccccc1] | 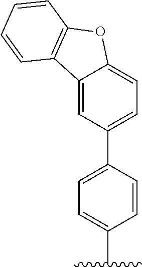 [SMILES: *c1ccc(-c2ccc3oc4ccccc4c3c2)cc1] |
| J31 | 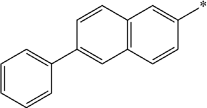 [SMILES: *c1ccc2cc(-c3ccccc3)ccc2c1] | 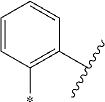 [SMILES: *c1ccccc1*] | 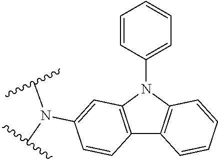 [SMILES: *N(*)c1ccc2c3ccccc3n(-c3ccccc3)c2c1] | 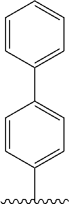 [SMILES: *c1ccc(-c2ccccc2)cc1] |
| J32 | 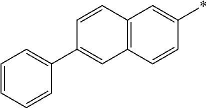 [SMILES: *c1ccc2cc(-c3ccccc3)ccc2c1] | 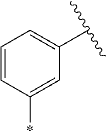 [SMILES: *c1cccc(*)c1] | 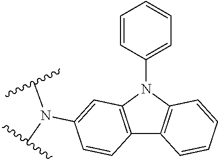 [SMILES: *N(*)c1ccc2c3ccccc3n(-c3ccccc3)c2c1] | 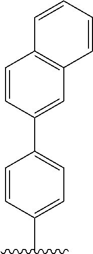 [SMILES: *c1ccc(-c2ccc3ccccc3c2)cc1] |
| J33 | 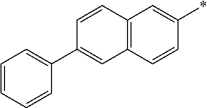 [SMILES: *c1ccc2cc(-c3ccccc3)ccc2c1] | 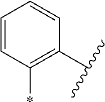 [SMILES: *c1ccccc1*] | 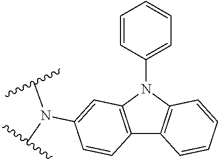 [SMILES: *N(*)c1ccc2c3ccccc3n(-c3ccccc3)c2c1] | 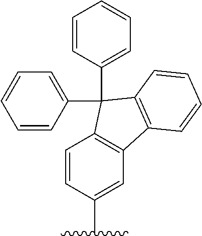 [SMILES: *c1ccc2c(c1)-c1ccccc1C2(c1ccccc1)c1ccccc1] |
| J34 | 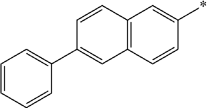 [SMILES: *c1ccc2cc(-c3ccccc3)ccc2c1] | 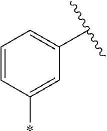 [SMILES: *c1cccc(*)c1] | 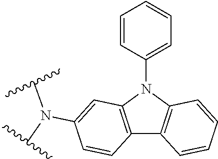 [SMILES: *N(*)c1ccc2c3ccccc3n(-c3ccccc3)c2c1] | 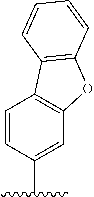 [SMILES: *c1ccc2c(c1)oc1ccccc12] |
| J35 | 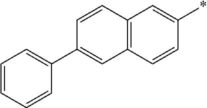 [SMILES: *c1ccc2cc(-c3ccccc3)ccc2c1] | 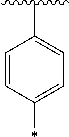 [SMILES: *c1ccc(*)cc1] | 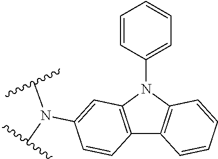 [SMILES: *N(*)c1ccc2c3ccccc3n(-c3ccccc3)c2c1] | 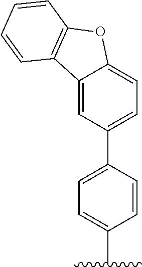 [SMILES: *c1ccc(-c2ccc3oc4ccccc4c3c2)cc1] |
| J36 | 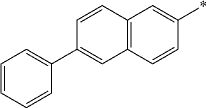 [SMILES: *c1ccc2cc(-c3ccccc3)ccc2c1] | 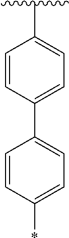 [SMILES: *c1ccc(-c2ccc(*)cc2)cc1] | 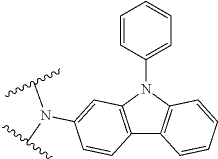 [SMILES: *N(*)c1ccc2c3ccccc3n(-c3ccccc3)c2c1] | 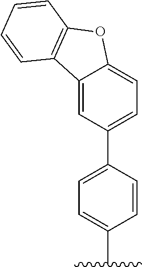 [SMILES: *c1ccc(-c2ccc3oc4ccccc4c3c2)cc1] |
| J37 | 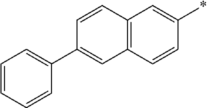 [SMILES: *c1ccc2cc(-c3ccccc3)ccc2c1] | 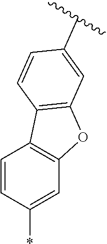 [SMILES: *c1ccc2c(c1)oc1cc(*)ccc12] | 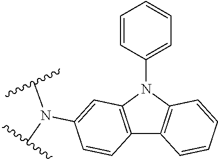 [SMILES: *N(*)c1ccc2c3ccccc3n(-c3ccccc3)c2c1] | 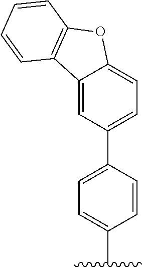 [SMILES: *c1ccc(-c2ccc3oc4ccccc4c3c2)cc1] |
| K1 | 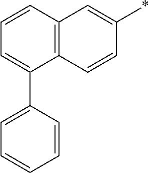 [SMILES: *c1ccc2c(-c3ccccc3)cccc2c1] | 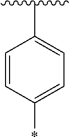 [SMILES: *c1ccc(*)cc1] | 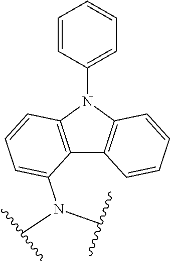 [SMILES: *N(*)c1cccc2c1c1ccccc1n2-c1ccccc1] | 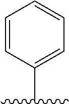 [SMILES: *c1ccccc1] |
| K2 | 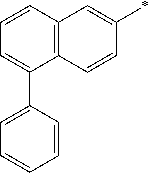 [SMILES: *c1ccc2c(-c3ccccc3)cccc2c1] | 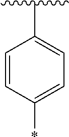 [SMILES: *c1ccc(*)cc1] | 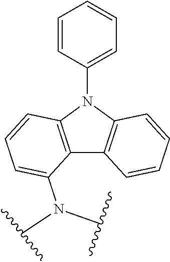 [SMILES: *N(*)c1cccc2c1c1ccccc1n2-c1ccccc1] | 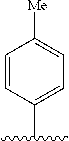 [SMILES: *c1ccc(C)cc1] |
| K3 | 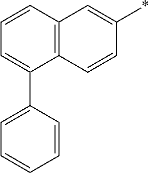 [SMILES: *c1ccc2c(-c3ccccc3)cccc2c1] | 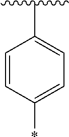 [SMILES: *c1ccc(*)cc1] | 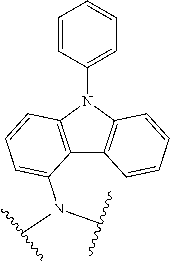 [SMILES: *N(*)c1cccc2c1c1ccccc1n2-c1ccccc1] | 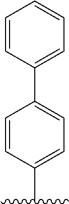 [SMILES: *c1ccc(-c2ccccc2)cc1] |
| K4 | 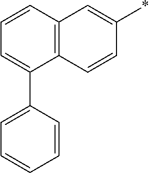 [SMILES: *c1ccc2c(-c3ccccc3)cccc2c1] | 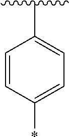 [SMILES: *c1ccc(*)cc1] | 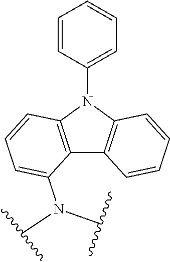 [SMILES: *N(*)c1cccc2c1c1ccccc1n2-c1ccccc1] | 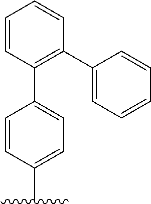 [SMILES: *c1ccc(-c2ccccc2-c2ccccc2)cc1] |
| K5 | 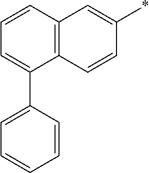 [SMILES: *c1ccc2c(-c3ccccc3)cccc2c1] | 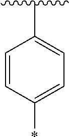 [SMILES: *c1ccc(*)cc1] | 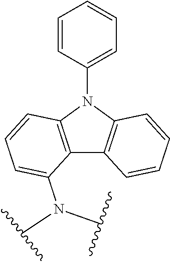 [SMILES: *N(*)c1cccc2c1c1ccccc1n2-c1ccccc1] | 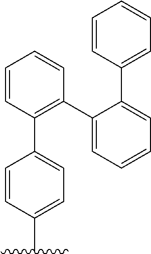 [SMILES: *c1ccc(-c2ccccc2-c2ccccc2-c2ccccc2)cc1] |
| K6 | 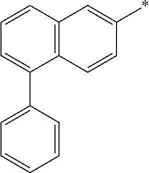 [SMILES: *c1ccc2c(-c3ccccc3)cccc2c1] | 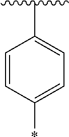 [SMILES: *c1ccc(*)cc1] | 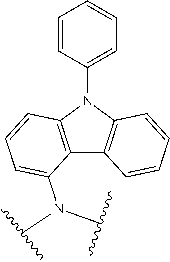 [SMILES: *N(*)c1cccc2c1c1ccccc1n2-c1ccccc1] | 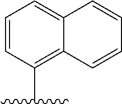 [SMILES: *c1cccc2ccccc12] |
| K7 | 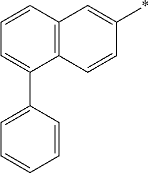 [SMILES: *c1ccc2c(-c3ccccc3)cccc2c1] | 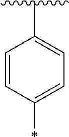 [SMILES: *c1ccc(*)cc1] | 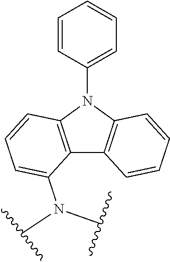 [SMILES: *N(*)c1cccc2c1c1ccccc1n2-c1ccccc1] | 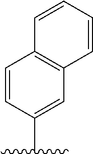 [SMILES: *c1ccc2ccccc2c1] |
| K8 | 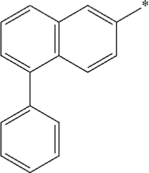 [SMILES: *c1ccc2c(-c3ccccc3)cccc2c1] | 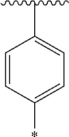 [SMILES: *c1ccc(*)cc1] | 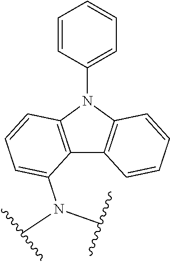 [SMILES: *N(*)c1cccc2c1c1ccccc1n2-c1ccccc1] | 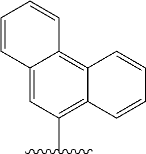 [SMILES: *c1cc2ccccc2c2ccccc12] |
| K9 | 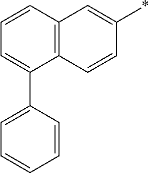 [SMILES: *c1ccc2c(-c3ccccc3)cccc2c1] | 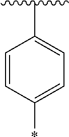 [SMILES: *c1ccc(*)cc1] | 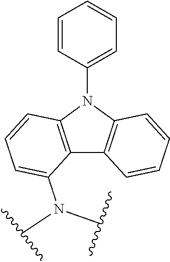 [SMILES: *N(*)c1cccc2c1c1ccccc1n2-c1ccccc1] | 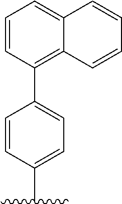 [SMILES: *c1ccc(-c2cccc3ccccc23)cc1] |
| K10 | 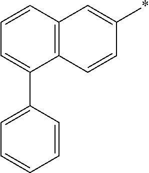 [SMILES: *c1ccc2c(-c3ccccc3)cccc2c1] | 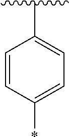 [SMILES: *c1ccc(*)cc1] | 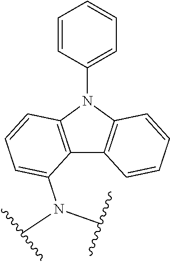 [SMILES: *N(*)c1cccc2c1c1ccccc1n2-c1ccccc1] | 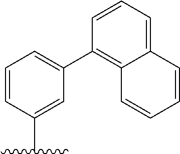 [SMILES: *c1cccc(-c2cccc3ccccc23)c1] |
| K11 | 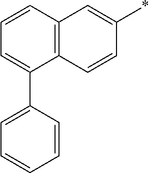 [SMILES: *c1ccc2c(-c3ccccc3)cccc2c1] | 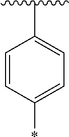 [SMILES: *c1ccc(*)cc1] | 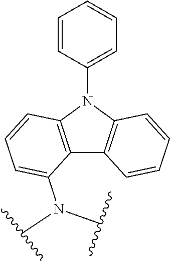 [SMILES: *N(*)c1cccc2c1c1ccccc1n2-c1ccccc1] | 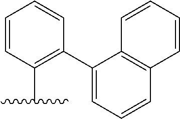 [SMILES: *c1ccccc1-c1cccc2ccccc12] |
| K12 | 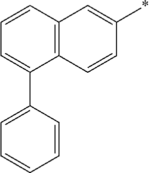 [SMILES: *c1ccc2c(-c3ccccc3)cccc2c1] | 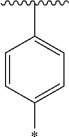 [SMILES: *c1ccc(*)cc1] | 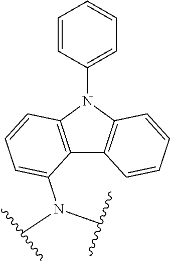 [SMILES: *N(*)c1cccc2c1c1ccccc1n2-c1ccccc1] | 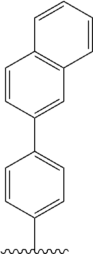 [SMILES: *c1ccc(-c2ccc3ccccc3c2)cc1] |
| K13 | 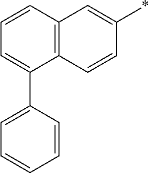 [SMILES: *c1ccc2c(-c3ccccc3)cccc2c1] | 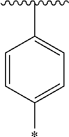 [SMILES: *c1ccc(*)cc1] | 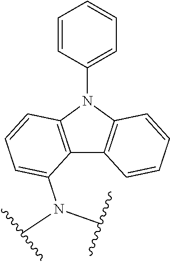 [SMILES: *N(*)c1cccc2c1c1ccccc1n2-c1ccccc1] | 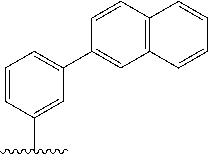 [SMILES: *c1cccc(-c2ccc3ccccc3c2)c1] |
| K14 | 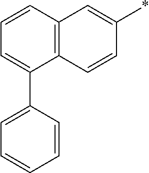 [SMILES: *c1ccc2c(-c3ccccc3)cccc2c1] | 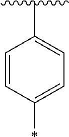 [SMILES: *c1ccc(*)cc1] | 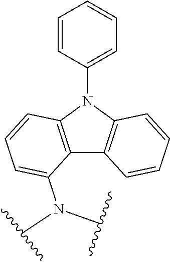 [SMILES: *N(*)c1cccc2c1c1ccccc1n2-c1ccccc1] | 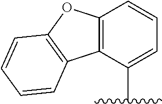 [SMILES: *c1cccc2oc3ccccc3c12] |
| K15 | 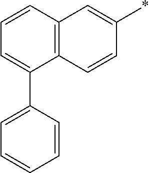 [SMILES: *c1ccc2c(-c3ccccc3)cccc2c1] | 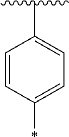 [SMILES: *c1ccc(*)cc1] | 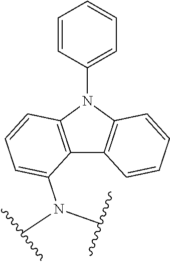 [SMILES: *N(*)c1cccc2c1c1ccccc1n2-c1ccccc1] | 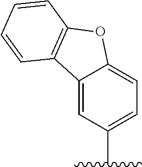 [SMILES: *c1ccc2oc3ccccc3c2c1] |
| K16 | 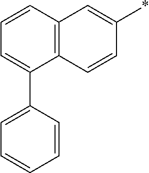 [SMILES: *c1ccc2c(-c3ccccc3)cccc2c1] | 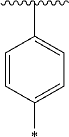 [SMILES: *c1ccc(*)cc1] | 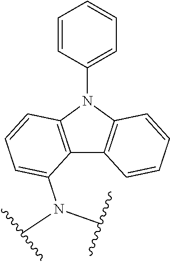 [SMILES: *N(*)c1cccc2c1c1ccccc1n2-c1ccccc1] | 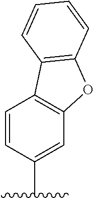 [SMILES: *c1ccc2c(c1)oc1ccccc12] |
| K17 | 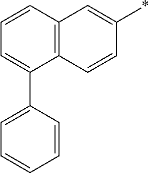 [SMILES: *c1ccc2c(-c3ccccc3)cccc2c1] | 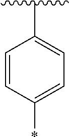 [SMILES: *c1ccc(*)cc1] | 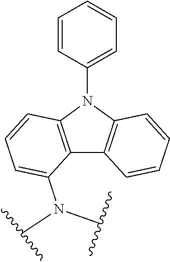 [SMILES: *N(*)c1cccc2c1c1ccccc1n2-c1ccccc1] | 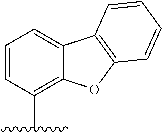 [SMILES: *c1cccc2c1oc1ccccc12] |
| K18 | 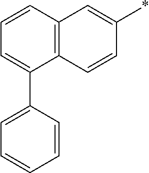 [SMILES: *c1ccc2c(-c3ccccc3)cccc2c1] | 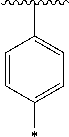 [SMILES: *c1ccc(*)cc1] | 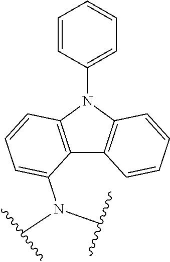 [SMILES: *N(*)c1cccc2c1c1ccccc1n2-c1ccccc1] | 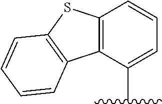 [SMILES: *c1cccc2sc3ccccc3c12] |
| K19 | 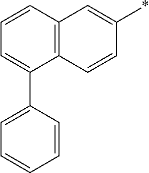 [SMILES: *c1ccc2c(-c3ccccc3)cccc2c1] | 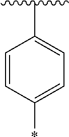 [SMILES: *c1ccc(*)cc1] | 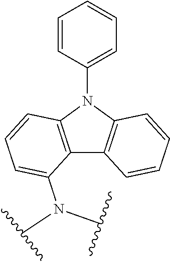 [SMILES: *N(*)c1cccc2c1c1ccccc1n2-c1ccccc1] | 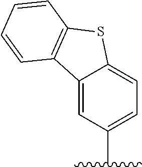 [SMILES: *c1ccc2sc3ccccc3c2c1] |
| K20 | 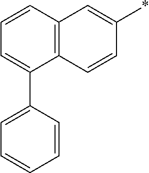 [SMILES: *c1ccc2c(-c3ccccc3)cccc2c1] | 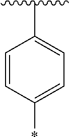 [SMILES: *c1ccc(*)cc1] | 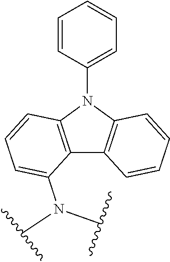 [SMILES: *N(*)c1cccc2c1c1ccccc1n2-c1ccccc1] | 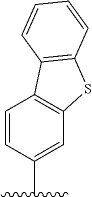 [SMILES: *c1ccc2c(c1)sc1ccccc12] |
| K21 | 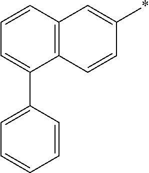 [SMILES: *c1ccc2c(-c3ccccc3)cccc2c1] | 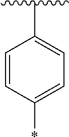 [SMILES: *c1ccc(*)cc1] | 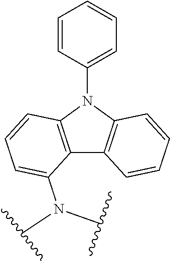 [SMILES: *N(*)c1cccc2c1c1ccccc1n2-c1ccccc1] | 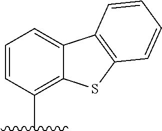 [SMILES: *c1cccc2c1sc1ccccc12] |
| K22 | 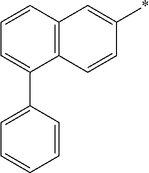 [SMILES: *c1ccc2c(-c3ccccc3)cccc2c1] | 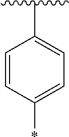 [SMILES: *c1ccc(*)cc1] | 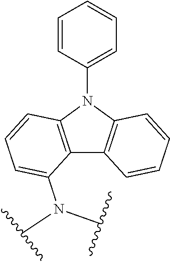 [SMILES: *N(*)c1cccc2c1c1ccccc1n2-c1ccccc1] | 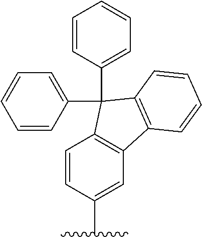 [SMILES: *c1ccc2c(c1)-c1ccccc1C2(c1ccccc1)c1ccccc1] |
| K23 | 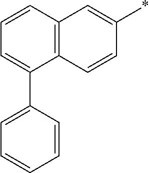 [SMILES: *c1ccc2c(-c3ccccc3)cccc2c1] | 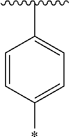 [SMILES: *c1ccc(*)cc1] | 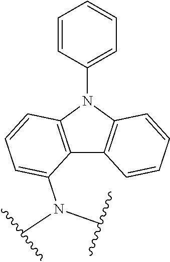 [SMILES: *N(*)c1cccc2c1c1ccccc1n2-c1ccccc1] | 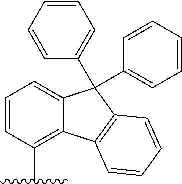 [SMILES: *c1cccc2c1-c1ccccc1C2(c1ccccc1)c1ccccc1] |
| K24 | 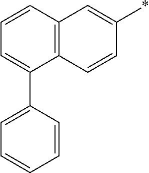 [SMILES: *c1ccc2c(-c3ccccc3)cccc2c1] | 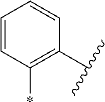 [SMILES: *c1ccccc1*] | 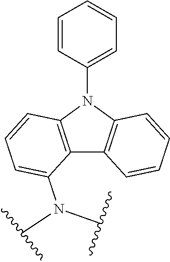 [SMILES: *N(*)c1cccc2c1c1ccccc1n2-c1ccccc1] | 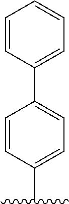 [SMILES: *c1ccc(-c2ccccc2)cc1] |
| K25 | 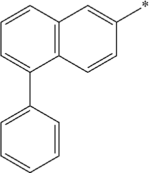 [SMILES: *c1ccc2c(-c3ccccc3)cccc2c1] | 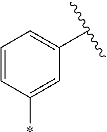 [SMILES: *c1cccc(*)c1] | 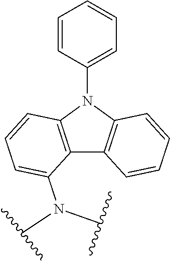 [SMILES: *N(*)c1cccc2c1c1ccccc1n2-c1ccccc1] | 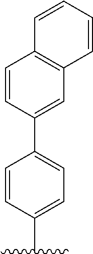 [SMILES: *c1ccc(-c2ccc3ccccc3c2)cc1] |
| K26 | 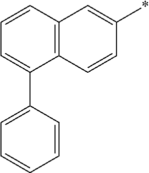 [SMILES: *c1ccc2c(-c3ccccc3)cccc2c1] | 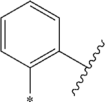 [SMILES: *c1ccccc1*] | 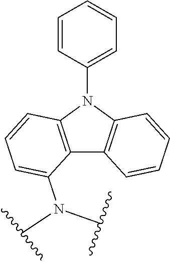 [SMILES: *N(*)c1cccc2c1c1ccccc1n2-c1ccccc1] | 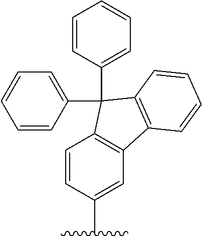 [SMILES: *c1ccc2c(c1)-c1ccccc1C2(c1ccccc1)c1ccccc1] |
| K27 | 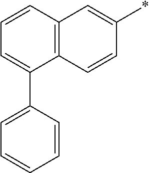 [SMILES: *c1ccc2c(-c3ccccc3)cccc2c1] | 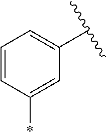 [SMILES: *c1cccc(*)c1] | 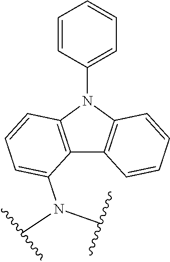 [SMILES: *N(*)c1cccc2c1c1ccccc1n2-c1ccccc1] | 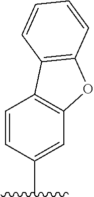 [SMILES: *c1ccc2c(c1)oc1ccccc12] |
| K28 | 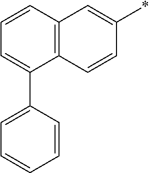 [SMILES: *c1ccc2c(-c3ccccc3)cccc2c1] | 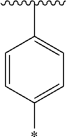 [SMILES: *c1ccc(*)cc1] | 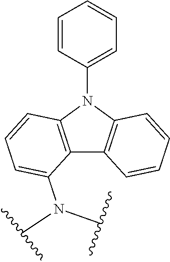 [SMILES: *N(*)c1cccc2c1c1ccccc1n2-c1ccccc1] | 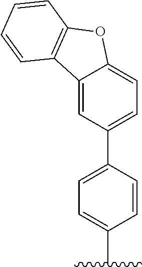 [SMILES: *c1ccc(-c2ccc3oc4ccccc4c3c2)cc1] |
| K29 | 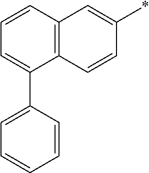 [SMILES: *c1ccc2c(-c3ccccc3)cccc2c1] | 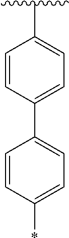 [SMILES: *c1ccc(-c2ccc(*)cc2)cc1] | 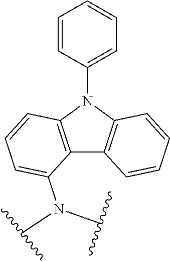 [SMILES: *N(*)c1cccc2c1c1ccccc1n2-c1ccccc1] | 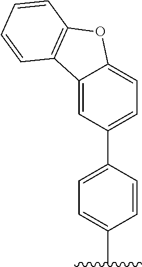 [SMILES: *c1ccc(-c2ccc3oc4ccccc4c3c2)cc1] |
| K30 | 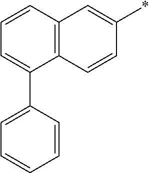 [SMILES: *c1ccc2c(-c3ccccc3)cccc2c1] | 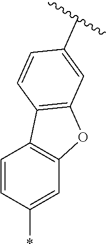 [SMILES: *c1ccc2c(c1)oc1cc(*)ccc12] | 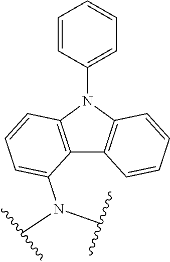 [SMILES: *N(*)c1cccc2c1c1ccccc1n2-c1ccccc1] | 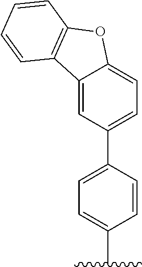 [SMILES: *c1ccc(-c2ccc3oc4ccccc4c3c2)cc1] |
| K31 | 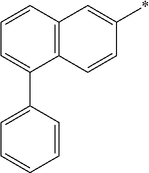 [SMILES: *c1ccc2c(-c3ccccc3)cccc2c1] | 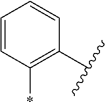 [SMILES: *c1ccccc1*] | 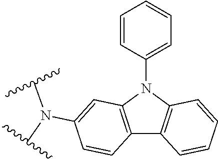 [SMILES: *N(*)c1ccc2c3ccccc3n(-c3ccccc3)c2c1] | 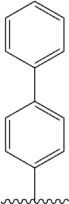 [SMILES: *c1ccc(-c2ccccc2)cc1] |
| K32 | 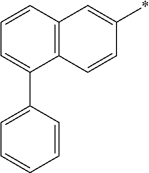 [SMILES: *c1ccc2c(-c3ccccc3)cccc2c1] | 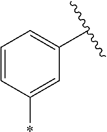 [SMILES: *c1cccc(*)c1] | 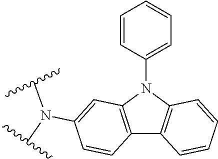 [SMILES: *N(*)c1ccc2c3ccccc3n(-c3ccccc3)c2c1] | 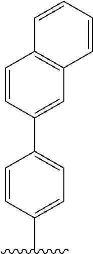 [SMILES: *c1ccc(-c2ccc3ccccc3c2)cc1] |
| K33 | 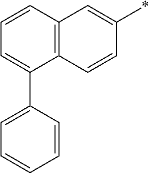 [SMILES: *c1ccc2c(-c3ccccc3)cccc2c1] | 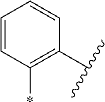 [SMILES: *c1ccccc1*] | 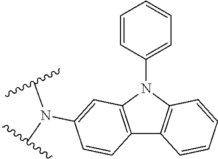 [SMILES: *N(*)c1ccc2c3ccccc3n(-c3ccccc3)c2c1] | 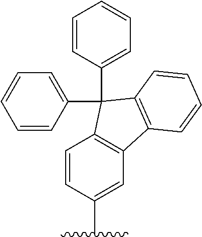 [SMILES: *c1ccc2c(c1)-c1ccccc1C2(c1ccccc1)c1ccccc1] |
| K34 | 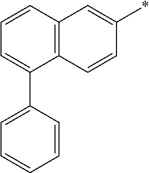 [SMILES: *c1ccc2c(-c3ccccc3)cccc2c1] | 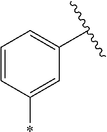 [SMILES: *c1cccc(*)c1] | 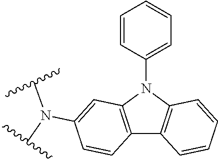 [SMILES: *N(*)c1ccc2c3ccccc3n(-c3ccccc3)c2c1] | 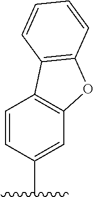 [SMILES: *c1ccc2c(c1)oc1ccccc12] |
| K35 | 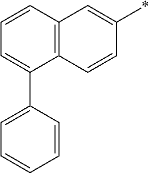 [SMILES: *c1ccc2c(-c3ccccc3)cccc2c1] | 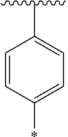 [SMILES: *c1ccc(*)cc1] | 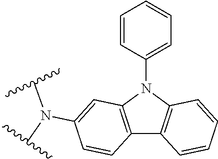 [SMILES: *N(*)c1ccc2c3ccccc3n(-c3ccccc3)c2c1] | 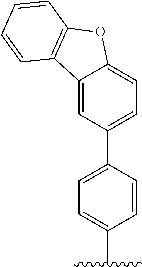 [SMILES: *c1ccc(-c2ccc3oc4ccccc4c3c2)cc1] |
| K36 | 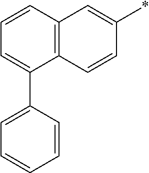 [SMILES: *c1ccc2c(-c3ccccc3)cccc2c1] | 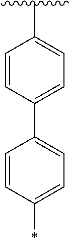 [SMILES: *c1ccc(-c2ccc(*)cc2)cc1] | 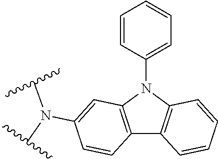 [SMILES: *N(*)c1ccc2c3ccccc3n(-c3ccccc3)c2c1] | 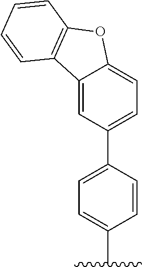 [SMILES: *c1ccc(-c2ccc3oc4ccccc4c3c2)cc1] |
| K37 | 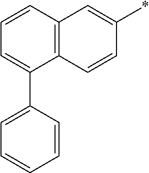 [SMILES: *c1ccc2c(-c3ccccc3)cccc2c1] | 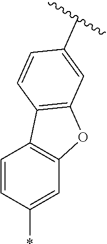 [SMILES: *c1ccc2c(c1)oc1cc(*)ccc12] | 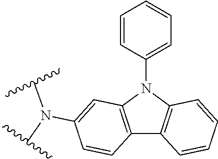 [SMILES: *N(*)c1ccc2c3ccccc3n(-c3ccccc3)c2c1] | 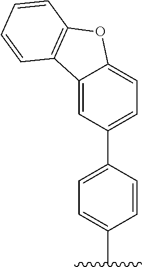 [SMILES: *c1ccc(-c2ccc3oc4ccccc4c3c2)cc1] |
| M1 | 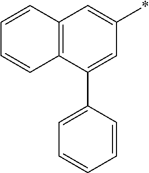 [SMILES: *c1cc(-c2ccccc2)c2ccccc2c1] | 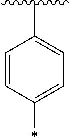 [SMILES: *c1ccc(*)cc1] | 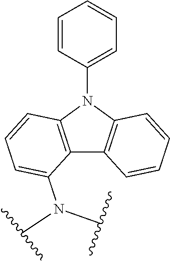 [SMILES: *N(*)c1cccc2c1c1ccccc1n2-c1ccccc1] | 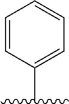 [SMILES: *c1ccccc1] |
| M2 | 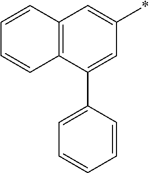 [SMILES: *c1cc(-c2ccccc2)c2ccccc2c1] | 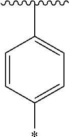 [SMILES: *c1ccc(*)cc1] | 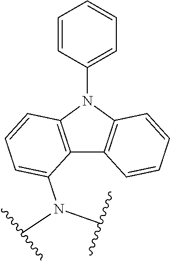 [SMILES: *N(*)c1cccc2c1c1ccccc1n2-c1ccccc1] | 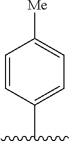 [SMILES: *c1ccc(C)cc1] |
| M3 | 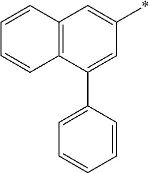 [SMILES: *c1cc(-c2ccccc2)c2ccccc2c1] | 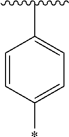 [SMILES: *c1ccc(*)cc1] | 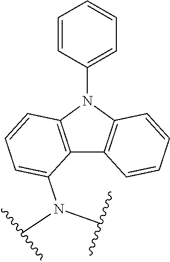 [SMILES: *N(*)c1cccc2c1c1ccccc1n2-c1ccccc1] | 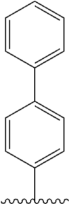 [SMILES: *c1ccc(-c2ccccc2)cc1] |
| M4 | 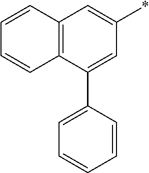 [SMILES: *c1cc(-c2ccccc2)c2ccccc2c1] | 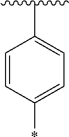 [SMILES: *c1ccc(*)cc1] | 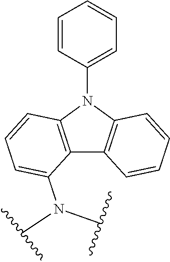 [SMILES: *N(*)c1cccc2c1c1ccccc1n2-c1ccccc1] | 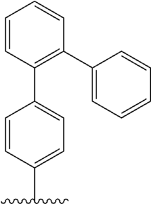 [SMILES: *c1ccc(-c2ccccc2-c2ccccc2)cc1] |
| M5 | 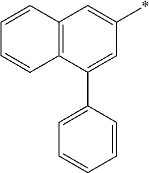 [SMILES: *c1cc(-c2ccccc2)c2ccccc2c1] | 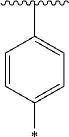 [SMILES: *c1ccc(*)cc1] | 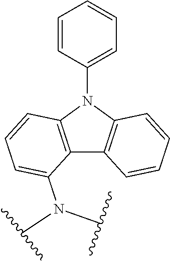 [SMILES: *N(*)c1cccc2c1c1ccccc1n2-c1ccccc1] | 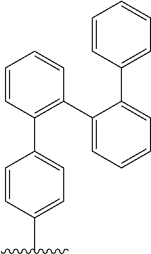 [SMILES: *c1ccc(-c2ccccc2-c2ccccc2-c2ccccc2)cc1] |
| M6 | 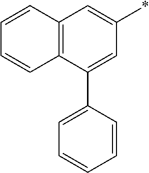 [SMILES: *c1cc(-c2ccccc2)c2ccccc2c1] | 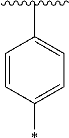 [SMILES: *c1ccc(*)cc1] | 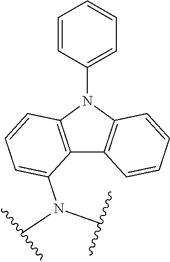 [SMILES: *N(*)c1cccc2c1c1ccccc1n2-c1ccccc1] | 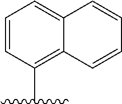 [SMILES: *c1cccc2ccccc12] |
| M7 | 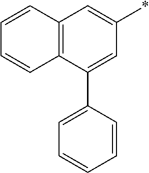 [SMILES: *c1cc(-c2ccccc2)c2ccccc2c1] | 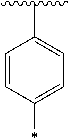 [SMILES: *c1ccc(*)cc1] | 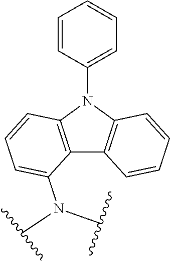 [SMILES: *N(*)c1cccc2c1c1ccccc1n2-c1ccccc1] | 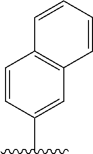 [SMILES: *c1ccc2ccccc2c1] |
| M8 | 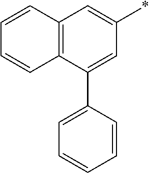 [SMILES: *c1cc(-c2ccccc2)c2ccccc2c1] | 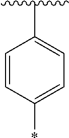 [SMILES: *c1ccc(*)cc1] | 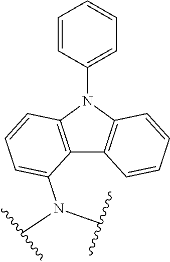 [SMILES: *N(*)c1cccc2c1c1ccccc1n2-c1ccccc1] | 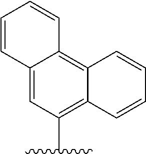 [SMILES: *c1cc2ccccc2c2ccccc12] |
| M9 | 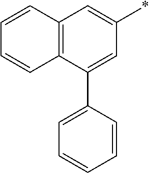 [SMILES: *c1cc(-c2ccccc2)c2ccccc2c1] | 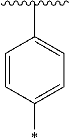 [SMILES: *c1ccc(*)cc1] | 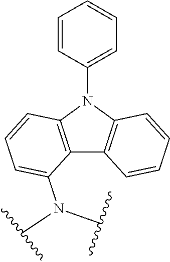 [SMILES: *N(*)c1cccc2c1c1ccccc1n2-c1ccccc1] | 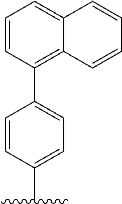 [SMILES: *c1ccc(-c2cccc3ccccc23)cc1] |
| M10 | 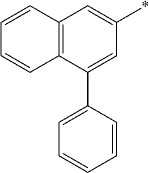 [SMILES: *c1cc(-c2ccccc2)c2ccccc2c1] | 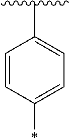 [SMILES: *c1ccc(*)cc1] | 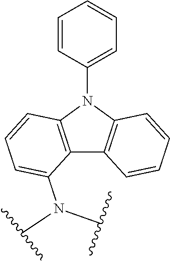 [SMILES: *N(*)c1cccc2c1c1ccccc1n2-c1ccccc1] | 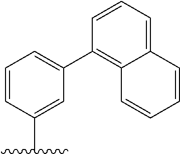 [SMILES: *c1cccc(-c2cccc3ccccc23)c1] |
| M11 | 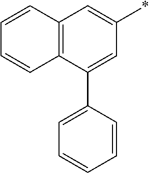 [SMILES: *c1cc(-c2ccccc2)c2ccccc2c1] | 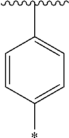 [SMILES: *c1ccc(*)cc1] | 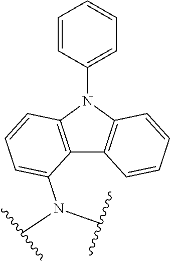 [SMILES: *N(*)c1cccc2c1c1ccccc1n2-c1ccccc1] | 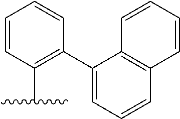 [SMILES: *c1ccccc1-c1cccc2ccccc12] |
| M12 | 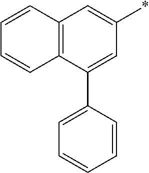 [SMILES: *c1cc(-c2ccccc2)c2ccccc2c1] | 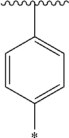 [SMILES: *c1ccc(*)cc1] | 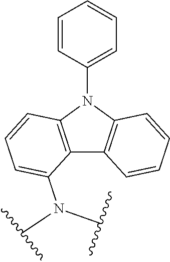 [SMILES: *N(*)c1cccc2c1c1ccccc1n2-c1ccccc1] | 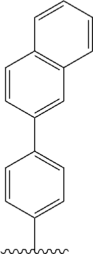 [SMILES: *c1ccc(-c2ccc3ccccc3c2)cc1] |
| M13 | 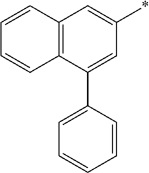 [SMILES: *c1cc(-c2ccccc2)c2ccccc2c1] | 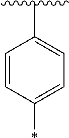 [SMILES: *c1ccc(*)cc1] | 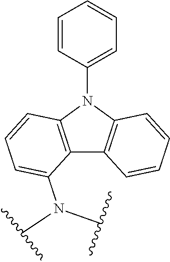 [SMILES: *N(*)c1cccc2c1c1ccccc1n2-c1ccccc1] | 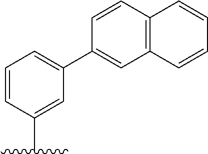 [SMILES: *c1cccc(-c2ccc3ccccc3c2)c1] |
| M14 | 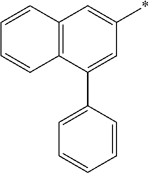 [SMILES: *c1cc(-c2ccccc2)c2ccccc2c1] | 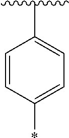 [SMILES: *c1ccc(*)cc1] | 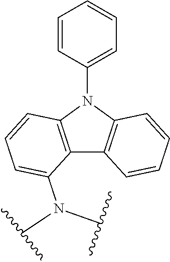 [SMILES: *N(*)c1cccc2c1c1ccccc1n2-c1ccccc1] | 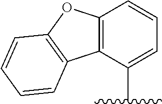 [SMILES: *c1cccc2oc3ccccc3c12] |
| M15 | 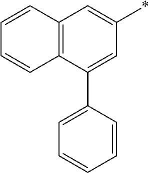 [SMILES: *c1cc(-c2ccccc2)c2ccccc2c1] | 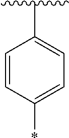 [SMILES: *c1ccc(*)cc1] | 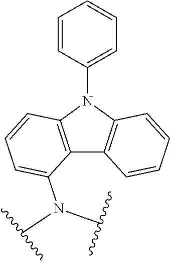 [SMILES: *N(*)c1cccc2c1c1ccccc1n2-c1ccccc1] | 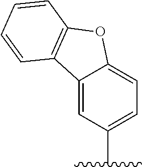 [SMILES: *c1ccc2oc3ccccc3c2c1] |
| M16 | 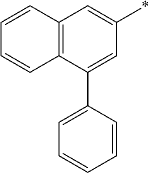 [SMILES: *c1cc(-c2ccccc2)c2ccccc2c1] | 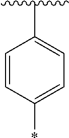 [SMILES: *c1ccc(*)cc1] | 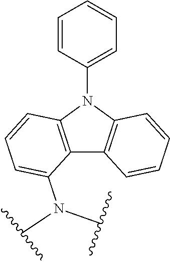 [SMILES: *N(*)c1cccc2c1c1ccccc1n2-c1ccccc1] | 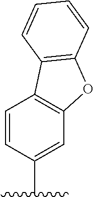 [SMILES: *c1ccc2c(c1)oc1ccccc12] |
| M17 | 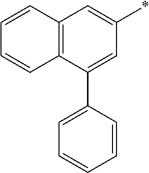 [SMILES: *c1cc(-c2ccccc2)c2ccccc2c1] | 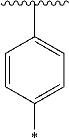 [SMILES: *c1ccc(*)cc1] | 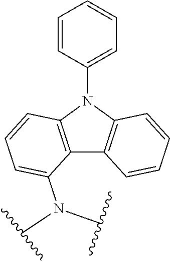 [SMILES: *N(*)c1cccc2c1c1ccccc1n2-c1ccccc1] | 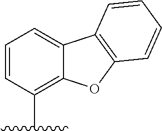 [SMILES: *c1cccc2c1oc1ccccc12] |
| M18 | 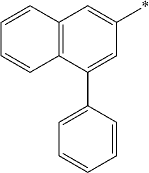 [SMILES: *c1cc(-c2ccccc2)c2ccccc2c1] | 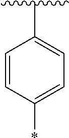 [SMILES: *c1ccc(*)cc1] | 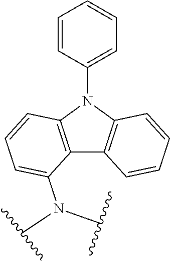 [SMILES: *N(*)c1cccc2c1c1ccccc1n2-c1ccccc1] | 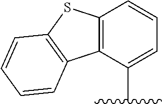 [SMILES: *c1cccc2sc3ccccc3c12] |
| M19 | 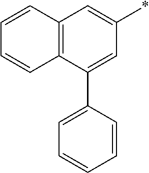 [SMILES: *c1cc(-c2ccccc2)c2ccccc2c1] | 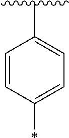 [SMILES: *c1ccc(*)cc1] | 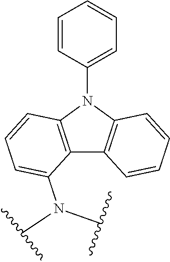 [SMILES: *N(*)c1cccc2c1c1ccccc1n2-c1ccccc1] | 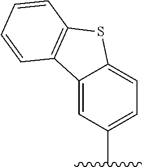 [SMILES: *c1ccc2sc3ccccc3c2c1] |
| M20 | 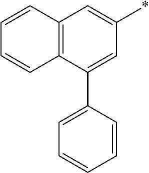 [SMILES: *c1cc(-c2ccccc2)c2ccccc2c1] | 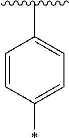 [SMILES: *c1ccc(*)cc1] | 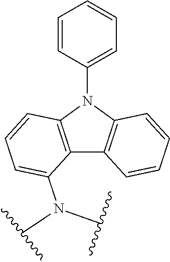 [SMILES: *N(*)c1cccc2c1c1ccccc1n2-c1ccccc1] | 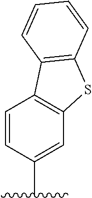 [SMILES: *c1ccc2c(c1)sc1ccccc12] |
| M21 | 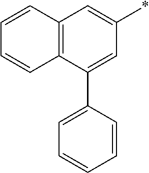 [SMILES: *c1cc(-c2ccccc2)c2ccccc2c1] | 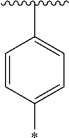 [SMILES: *c1ccc(*)cc1] | 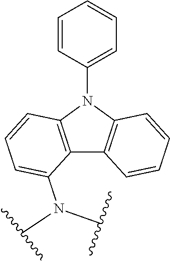 [SMILES: *N(*)c1cccc2c1c1ccccc1n2-c1ccccc1] | 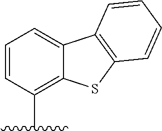 [SMILES: *c1cccc2c1sc1ccccc12] |
| M22 | 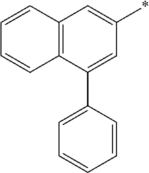 [SMILES: *c1cc(-c2ccccc2)c2ccccc2c1] | 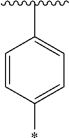 [SMILES: *c1ccc(*)cc1] | 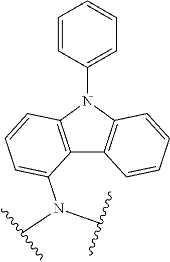 [SMILES: *N(*)c1cccc2c1c1ccccc1n2-c1ccccc1] | 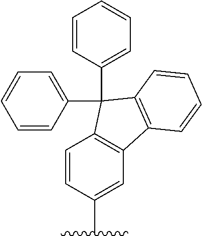 [SMILES: *c1ccc2c(c1)-c1ccccc1C2(c1ccccc1)c1ccccc1] |
| M23 | 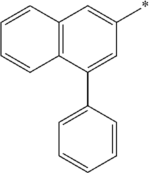 [SMILES: *c1cc(-c2ccccc2)c2ccccc2c1] | 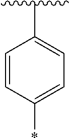 [SMILES: *c1ccc(*)cc1] | 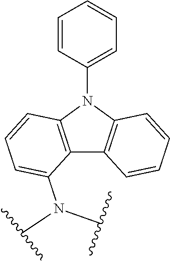 [SMILES: *N(*)c1cccc2c1c1ccccc1n2-c1ccccc1] | 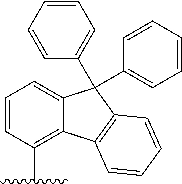 [SMILES: *c1cccc2c1-c1ccccc1C2(c1ccccc1)c1ccccc1] |
| M24 | 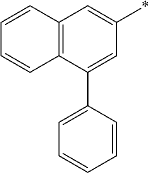 [SMILES: *c1cc(-c2ccccc2)c2ccccc2c1] | 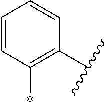 [SMILES: *c1ccccc1*] | 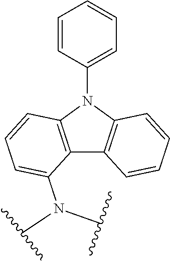 [SMILES: *N(*)c1cccc2c1c1ccccc1n2-c1ccccc1] | 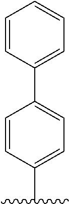 [SMILES: *c1ccc(-c2ccccc2)cc1] |
| M25 | 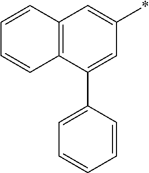 [SMILES: *c1cc(-c2ccccc2)c2ccccc2c1] | 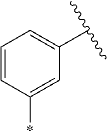 [SMILES: *c1cccc(*)c1] | 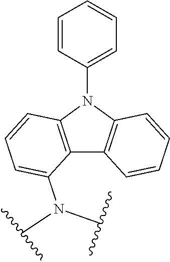 [SMILES: *N(*)c1cccc2c1c1ccccc1n2-c1ccccc1] | 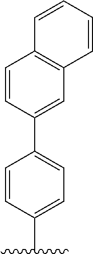 [SMILES: *c1ccc(-c2ccc3ccccc3c2)cc1] |
| M26 | 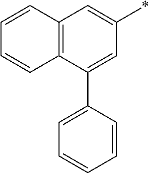 [SMILES: *c1cc(-c2ccccc2)c2ccccc2c1] | 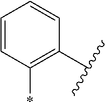 [SMILES: *c1ccccc1*] | 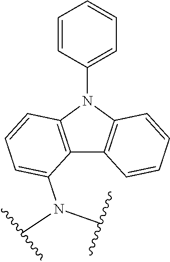 [SMILES: *N(*)c1cccc2c1c1ccccc1n2-c1ccccc1] | 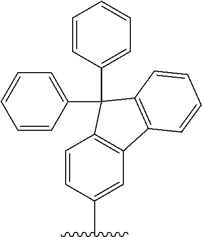 [SMILES: *c1ccc2c(c1)-c1ccccc1C2(c1ccccc1)c1ccccc1] |
| M27 | 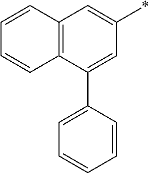 [SMILES: *c1cc(-c2ccccc2)c2ccccc2c1] | 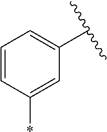 [SMILES: *c1cccc(*)c1] | 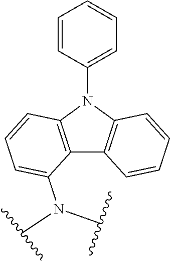 [SMILES: *N(*)c1cccc2c1c1ccccc1n2-c1ccccc1] | 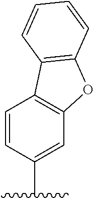 [SMILES: *c1ccc2c(c1)oc1ccccc12] |
| M28 | 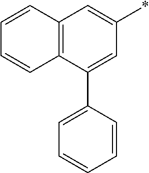 [SMILES: *c1cc(-c2ccccc2)c2ccccc2c1] | 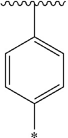 [SMILES: *c1ccc(*)cc1] | 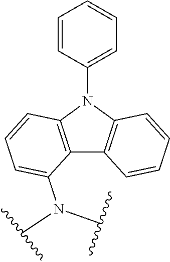 [SMILES: *N(*)c1cccc2c1c1ccccc1n2-c1ccccc1] | 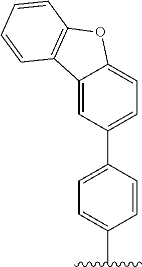 [SMILES: *c1ccc(-c2ccc3oc4ccccc4c3c2)cc1] |
| M29 | 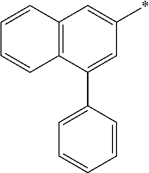 [SMILES: *c1cc(-c2ccccc2)c2ccccc2c1] | 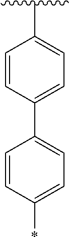 [SMILES: *c1ccc(-c2ccc(*)cc2)cc1] | 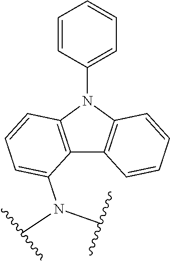 [SMILES: *N(*)c1cccc2c1c1ccccc1n2-c1ccccc1] | 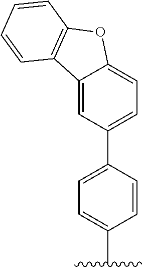 [SMILES: *c1ccc(-c2ccc3oc4ccccc4c3c2)cc1] |
| M31 | 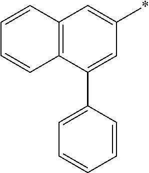 [SMILES: *c1cc(-c2ccccc2)c2ccccc2c1] | 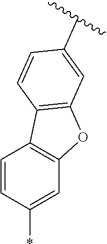 [SMILES: *c1ccc2c(c1)oc1cc(*)ccc12] | 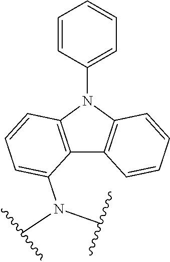 [SMILES: *N(*)c1cccc2c1c1ccccc1n2-c1ccccc1] | 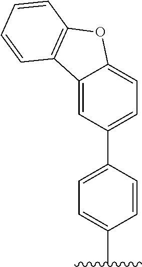 [SMILES: *c1ccc(-c2ccc3oc4ccccc4c3c2)cc1] |
| M31 | 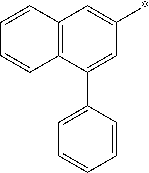 [SMILES: *c1cc(-c2ccccc2)c2ccccc2c1] | 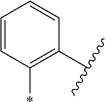 [SMILES: *c1ccccc1*] | 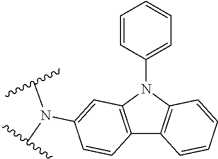 [SMILES: *N(*)c1ccc2c3ccccc3n(-c3ccccc3)c2c1] | 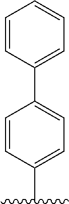 [SMILES: *c1ccc(-c2ccccc2)cc1] |
| M32 | 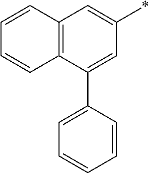 [SMILES: *c1cc(-c2ccccc2)c2ccccc2c1] | 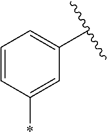 [SMILES: *c1cccc(*)c1] | 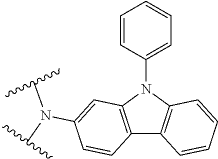 [SMILES: *N(*)c1ccc2c3ccccc3n(-c3ccccc3)c2c1] | 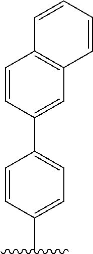 [SMILES: *c1ccc(-c2ccc3ccccc3c2)cc1] |
| M33 | 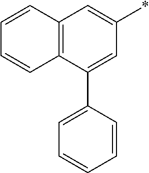 [SMILES: *c1cc(-c2ccccc2)c2ccccc2c1] | 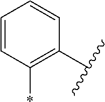 [SMILES: *c1ccccc1*] | 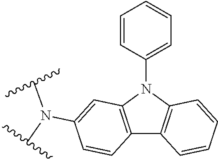 [SMILES: *N(*)c1ccc2c3ccccc3n(-c3ccccc3)c2c1] | 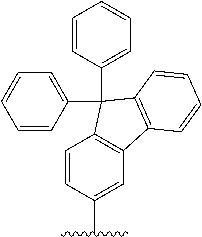 [SMILES: *c1ccc2c(c1)-c1ccccc1C2(c1ccccc1)c1ccccc1] |
| M34 | 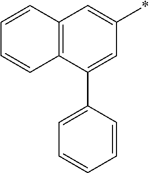 [SMILES: *c1cc(-c2ccccc2)c2ccccc2c1] | 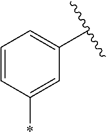 [SMILES: *c1cccc(*)c1] | 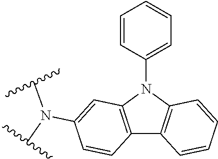 [SMILES: *N(*)c1ccc2c3ccccc3n(-c3ccccc3)c2c1] | 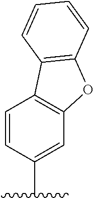 [SMILES: *c1ccc2c(c1)oc1ccccc12] |
| M35 | 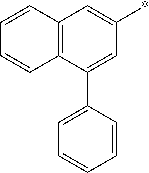 [SMILES: *c1cc(-c2ccccc2)c2ccccc2c1] | 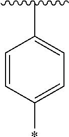 [SMILES: *c1ccc(*)cc1] | 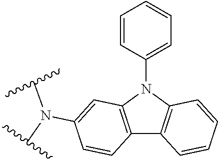 [SMILES: *N(*)c1ccc2c3ccccc3n(-c3ccccc3)c2c1] | 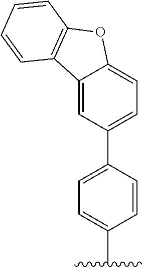 [SMILES: *c1ccc(-c2ccc3oc4ccccc4c3c2)cc1] |
| M36 | 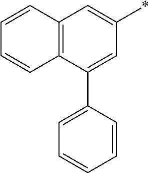 [SMILES: *c1cc(-c2ccccc2)c2ccccc2c1] | 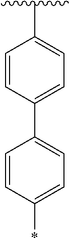 [SMILES: *c1ccc(-c2ccc(*)cc2)cc1] | 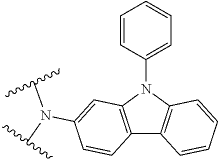 [SMILES: *N(*)c1ccc2c3ccccc3n(-c3ccccc3)c2c1] | 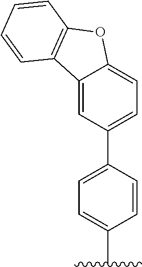 [SMILES: *c1ccc(-c2ccc3oc4ccccc4c3c2)cc1] |
| M37 | 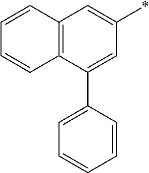 [SMILES: *c1cc(-c2ccccc2)c2ccccc2c1] | 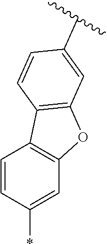 [SMILES: *c1ccc2c(c1)oc1cc(*)ccc12] | 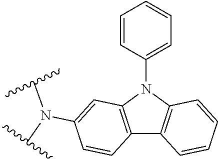 [SMILES: *N(*)c1ccc2c3ccccc3n(-c3ccccc3)c2c1] | 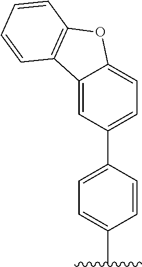 [SMILES: *c1ccc(-c2ccc3oc4ccccc4c3c2)cc1] |
| N1 | 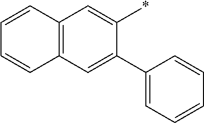 [SMILES: *c1cc2ccccc2cc1-c1ccccc1] | 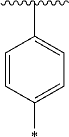 [SMILES: *c1ccc(*)cc1] | 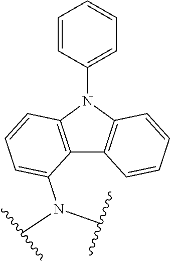 [SMILES: *N(*)c1cccc2c1c1ccccc1n2-c1ccccc1] | 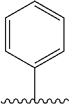 [SMILES: *c1ccccc1] |
| N2 | 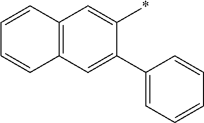 [SMILES: *c1cc2ccccc2cc1-c1ccccc1] | 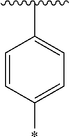 [SMILES: *c1ccc(*)cc1] | 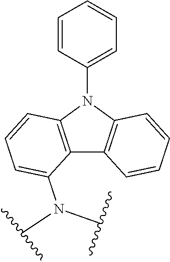 [SMILES: *N(*)c1cccc2c1c1ccccc1n2-c1ccccc1] | 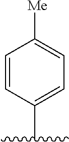 [SMILES: *c1ccc(C)cc1] |
| N3 | 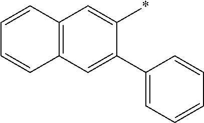 [SMILES: *c1cc2ccccc2cc1-c1ccccc1] | 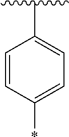 [SMILES: *c1ccc(*)cc1] | 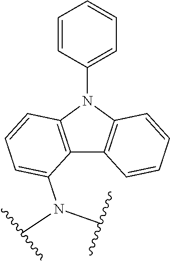 [SMILES: *N(*)c1cccc2c1c1ccccc1n2-c1ccccc1] | 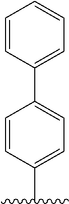 [SMILES: *c1ccc(-c2ccccc2)cc1] |
| N4 | 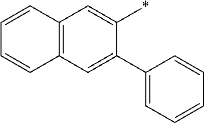 [SMILES: *c1cc2ccccc2cc1-c1ccccc1] | 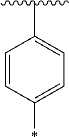 [SMILES: *c1ccc(*)cc1] | 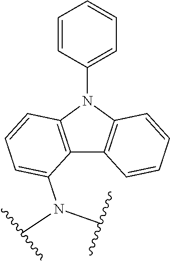 [SMILES: *N(*)c1cccc2c1c1ccccc1n2-c1ccccc1] | 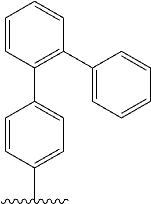 [SMILES: *c1ccc(-c2ccccc2-c2ccccc2)cc1] |
| N5 | 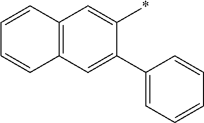 [SMILES: *c1cc2ccccc2cc1-c1ccccc1] | 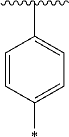 [SMILES: *c1ccc(*)cc1] | 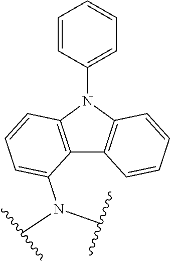 [SMILES: *N(*)c1cccc2c1c1ccccc1n2-c1ccccc1] | 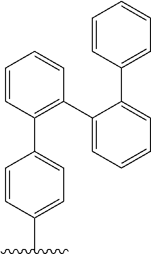 [SMILES: *c1ccc(-c2ccccc2-c2ccccc2-c2ccccc2)cc1] |
| N6 | 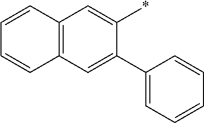 [SMILES: *c1cc2ccccc2cc1-c1ccccc1] | 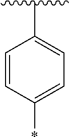 [SMILES: *c1ccc(*)cc1] | 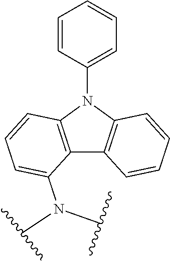 [SMILES: *N(*)c1cccc2c1c1ccccc1n2-c1ccccc1] | 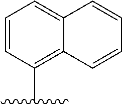 [SMILES: *c1cccc2ccccc12] |
| N7 | 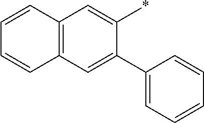 [SMILES: *c1cc2ccccc2cc1-c1ccccc1] | 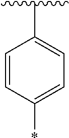 [SMILES: *c1ccc(*)cc1] | 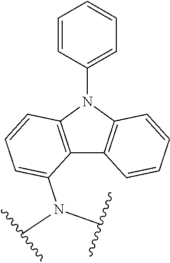 [SMILES: *N(*)c1cccc2c1c1ccccc1n2-c1ccccc1] | 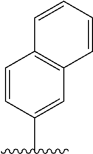 [SMILES: *c1ccc2ccccc2c1] |
| N8 | 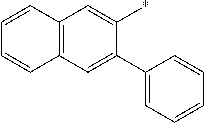 [SMILES: *c1cc2ccccc2cc1-c1ccccc1] | 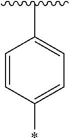 [SMILES: *c1ccc(*)cc1] | 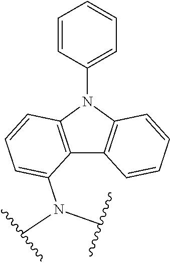 [SMILES: *N(*)c1cccc2c1c1ccccc1n2-c1ccccc1] | 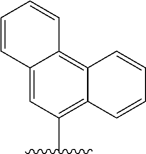 [SMILES: *c1cc2ccccc2c2ccccc12] |
| N9 | 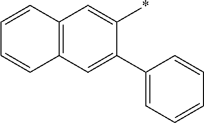 [SMILES: *c1cc2ccccc2cc1-c1ccccc1] | 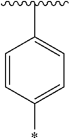 [SMILES: *c1ccc(*)cc1] | 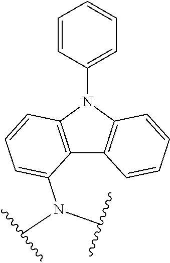 [SMILES: *N(*)c1cccc2c1c1ccccc1n2-c1ccccc1] | 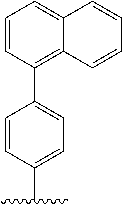 [SMILES: *c1ccc(-c2cccc3ccccc23)cc1] |
| N10 | 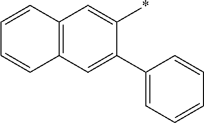 [SMILES: *c1cc2ccccc2cc1-c1ccccc1] | 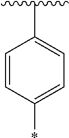 [SMILES: *c1ccc(*)cc1] | 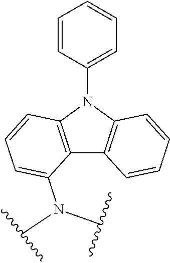 [SMILES: *N(*)c1cccc2c1c1ccccc1n2-c1ccccc1] | 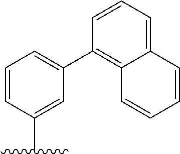 [SMILES: *c1cccc(-c2cccc3ccccc23)c1] |
| N11 | 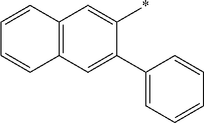 [SMILES: *c1cc2ccccc2cc1-c1ccccc1] | 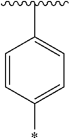 [SMILES: *c1ccc(*)cc1] | 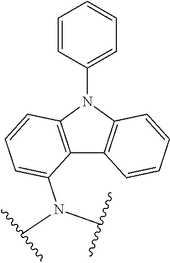 [SMILES: *N(*)c1cccc2c1c1ccccc1n2-c1ccccc1] | 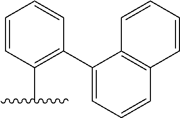 [SMILES: *c1ccccc1-c1cccc2ccccc12] |
| N12 | 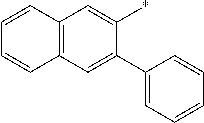 [SMILES: *c1cc2ccccc2cc1-c1ccccc1] | 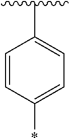 [SMILES: *c1ccc(*)cc1] | 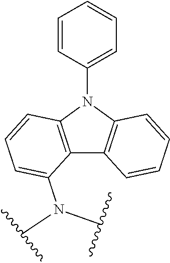 [SMILES: *N(*)c1cccc2c1c1ccccc1n2-c1ccccc1] | 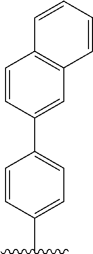 [SMILES: *c1ccc(-c2ccc3ccccc3c2)cc1] |
| N13 | 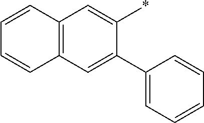 [SMILES: *c1cc2ccccc2cc1-c1ccccc1] | 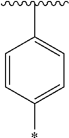 [SMILES: *c1ccc(*)cc1] | 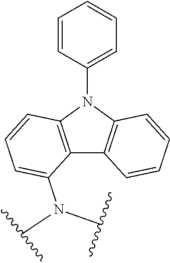 [SMILES: *N(*)c1cccc2c1c1ccccc1n2-c1ccccc1] | 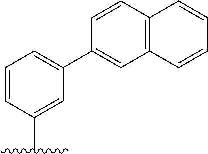 [SMILES: *c1cccc(-c2ccc3ccccc3c2)c1] |
| N14 | 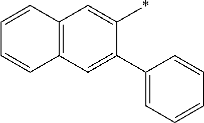 [SMILES: *c1cc2ccccc2cc1-c1ccccc1] | 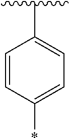 [SMILES: *c1ccc(*)cc1] | 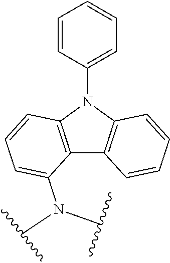 [SMILES: *N(*)c1cccc2c1c1ccccc1n2-c1ccccc1] | 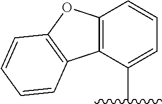 [SMILES: *c1cccc2oc3ccccc3c12] |
| N15 | 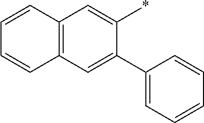 [SMILES: *c1cc2ccccc2cc1-c1ccccc1] | 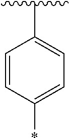 [SMILES: *c1ccc(*)cc1] | 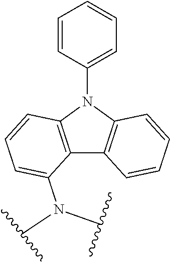 [SMILES: *N(*)c1cccc2c1c1ccccc1n2-c1ccccc1] | 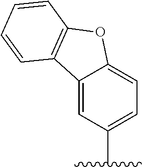 [SMILES: *c1ccc2oc3ccccc3c2c1] |
| N16 | 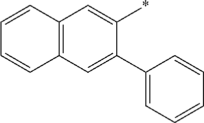 [SMILES: *c1cc2ccccc2cc1-c1ccccc1] | 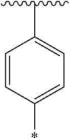 [SMILES: *c1ccc(*)cc1] | 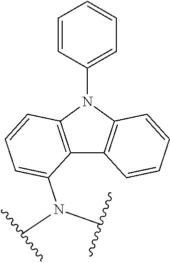 [SMILES: *N(*)c1cccc2c1c1ccccc1n2-c1ccccc1] | 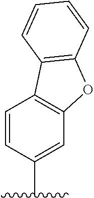 [SMILES: *c1ccc2c(c1)oc1ccccc12] |
| N17 | 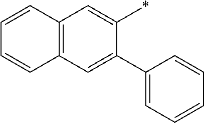 [SMILES: *c1cc2ccccc2cc1-c1ccccc1] | 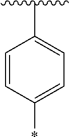 [SMILES: *c1ccc(*)cc1] | 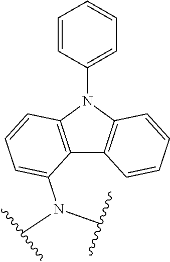 [SMILES: *N(*)c1cccc2c1c1ccccc1n2-c1ccccc1] | 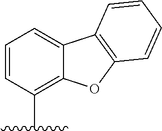 [SMILES: *c1cccc2c1oc1ccccc12] |
| N18 | 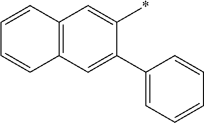 [SMILES: *c1cc2ccccc2cc1-c1ccccc1] | 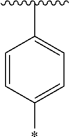 [SMILES: *c1ccc(*)cc1] | 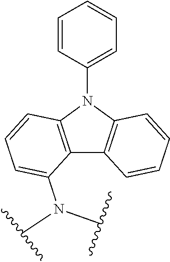 [SMILES: *N(*)c1cccc2c1c1ccccc1n2-c1ccccc1] | 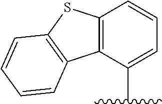 [SMILES: *c1cccc2sc3ccccc3c12] |
| N19 | 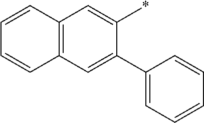 [SMILES: *c1cc2ccccc2cc1-c1ccccc1] | 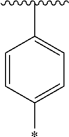 [SMILES: *c1ccc(*)cc1] | 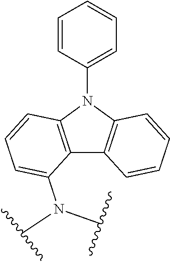 [SMILES: *N(*)c1cccc2c1c1ccccc1n2-c1ccccc1] | 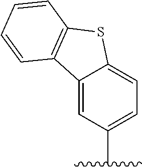 [SMILES: *c1ccc2sc3ccccc3c2c1] |
| N20 | 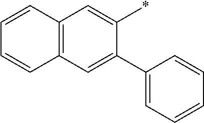 [SMILES: *c1cc2ccccc2cc1-c1ccccc1] | 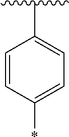 [SMILES: *c1ccc(*)cc1] | 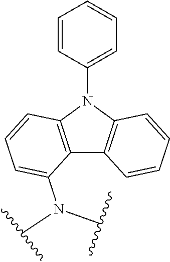 [SMILES: *N(*)c1cccc2c1c1ccccc1n2-c1ccccc1] | 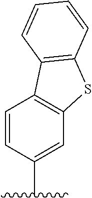 [SMILES: *c1ccc2c(c1)sc1ccccc12] |
| N21 | 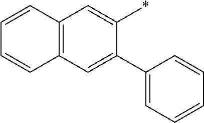 [SMILES: *c1cc2ccccc2cc1-c1ccccc1] | 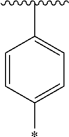 [SMILES: *c1ccc(*)cc1] | 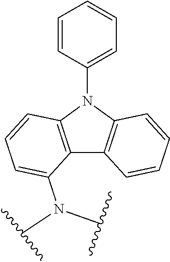 [SMILES: *N(*)c1cccc2c1c1ccccc1n2-c1ccccc1] | 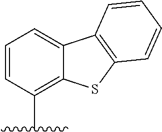 [SMILES: *c1cccc2c1sc1ccccc12] |
| N22 | 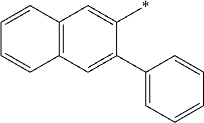 [SMILES: *c1cc2ccccc2cc1-c1ccccc1] | 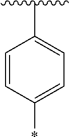 [SMILES: *c1ccc(*)cc1] | 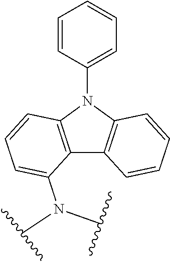 [SMILES: *N(*)c1cccc2c1c1ccccc1n2-c1ccccc1] | 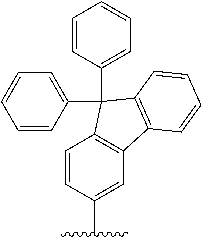 [SMILES: *c1ccc2c(c1)-c1ccccc1C2(c1ccccc1)c1ccccc1] |
| N23 | 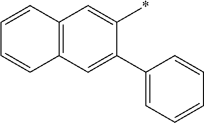 [SMILES: *c1cc2ccccc2cc1-c1ccccc1] | 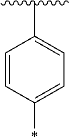 [SMILES: *c1ccc(*)cc1] | 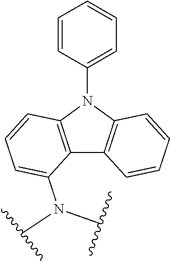 [SMILES: *N(*)c1cccc2c1c1ccccc1n2-c1ccccc1] | 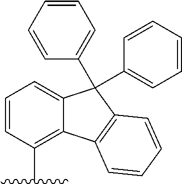 [SMILES: *c1cccc2c1-c1ccccc1C2(c1ccccc1)c1ccccc1] |
| N24 | 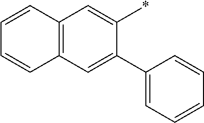 [SMILES: *c1cc2ccccc2cc1-c1ccccc1] | 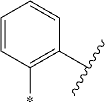 [SMILES: *c1ccccc1*] | 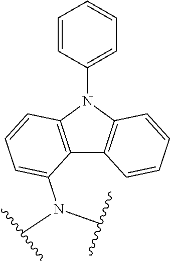 [SMILES: *N(*)c1cccc2c1c1ccccc1n2-c1ccccc1] | 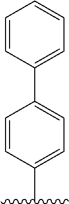 [SMILES: *c1ccc(-c2ccccc2)cc1] |
| N25 | 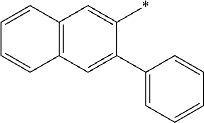 [SMILES: *c1cc2ccccc2cc1-c1ccccc1] | 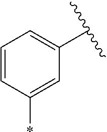 [SMILES: *c1cccc(*)c1] | 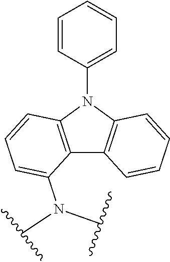 [SMILES: *N(*)c1cccc2c1c1ccccc1n2-c1ccccc1] | 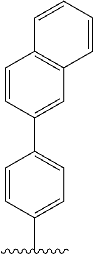 [SMILES: *c1ccc(-c2ccc3ccccc3c2)cc1] |
| N26 | 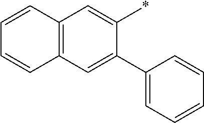 [SMILES: *c1cc2ccccc2cc1-c1ccccc1] | 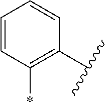 [SMILES: *c1ccccc1*] | 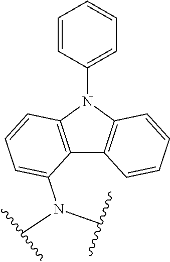 [SMILES: *N(*)c1cccc2c1c1ccccc1n2-c1ccccc1] | 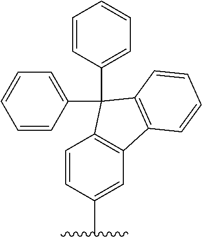 [SMILES: *c1ccc2c(c1)-c1ccccc1C2(c1ccccc1)c1ccccc1] |
| N27 | 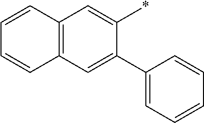 [SMILES: *c1cc2ccccc2cc1-c1ccccc1] | 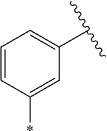 [SMILES: *c1cccc(*)c1] | 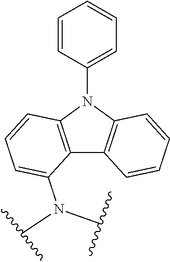 [SMILES: *N(*)c1cccc2c1c1ccccc1n2-c1ccccc1] | 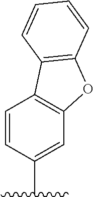 [SMILES: *c1ccc2c(c1)oc1ccccc12] |
| N28 | 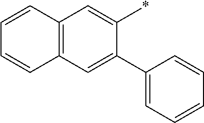 [SMILES: *c1cc2ccccc2cc1-c1ccccc1] | 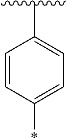 [SMILES: *c1ccc(*)cc1] | 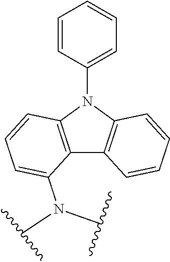 [SMILES: *N(*)c1cccc2c1c1ccccc1n2-c1ccccc1] | 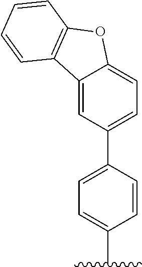 [SMILES: *c1ccc(-c2ccc3oc4ccccc4c3c2)cc1] |
| N29 | 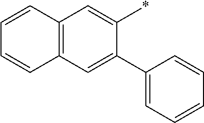 [SMILES: *c1cc2ccccc2cc1-c1ccccc1] | 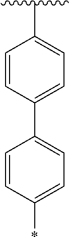 [SMILES: *c1ccc(-c2ccc(*)cc2)cc1] | 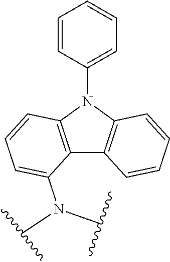 [SMILES: *N(*)c1cccc2c1c1ccccc1n2-c1ccccc1] | 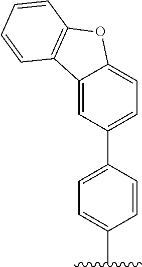 [SMILES: *c1ccc(-c2ccc3oc4ccccc4c3c2)cc1] |
| N30 | 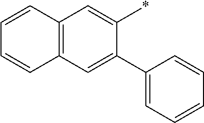 [SMILES: *c1cc2ccccc2cc1-c1ccccc1] | 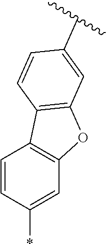 [SMILES: *c1ccc2c(c1)oc1cc(*)ccc12] | 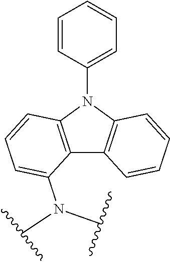 [SMILES: *N(*)c1cccc2c1c1ccccc1n2-c1ccccc1] | 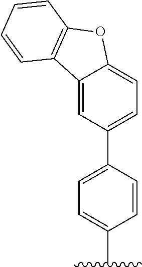 [SMILES: *c1ccc(-c2ccc3oc4ccccc4c3c2)cc1] |
| N31 | 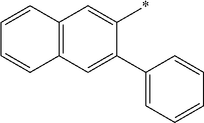 [SMILES: *c1cc2ccccc2cc1-c1ccccc1] | 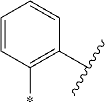 [SMILES: *c1ccccc1*] | 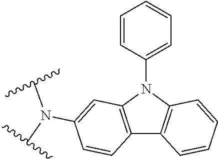 [SMILES: *N(*)c1ccc2c3ccccc3n(-c3ccccc3)c2c1] | 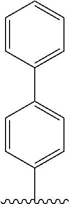 [SMILES: *c1ccc(-c2ccccc2)cc1] |
| N32 | 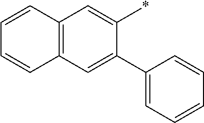 [SMILES: *c1cc2ccccc2cc1-c1ccccc1] | 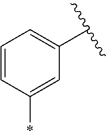 [SMILES: *c1cccc(*)c1] | 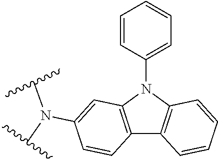 [SMILES: *N(*)c1ccc2c3ccccc3n(-c3ccccc3)c2c1] | 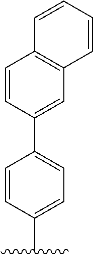 [SMILES: *c1ccc(-c2ccc3ccccc3c2)cc1] |
| N33 | 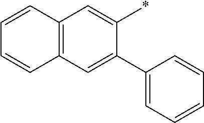 [SMILES: *c1cc2ccccc2cc1-c1ccccc1] | 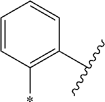 [SMILES: *c1ccccc1*] | 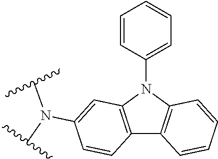 [SMILES: *N(*)c1ccc2c3ccccc3n(-c3ccccc3)c2c1] | 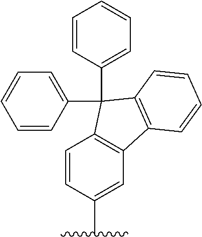 [SMILES: *c1ccc2c(c1)-c1ccccc1C2(c1ccccc1)c1ccccc1] |
| N34 | 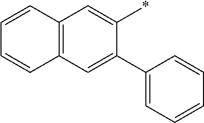 [SMILES: *c1cc2ccccc2cc1-c1ccccc1] | 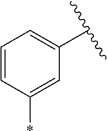 [SMILES: *c1cccc(*)c1] | 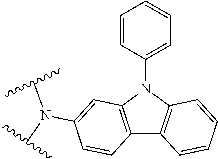 [SMILES: *N(*)c1ccc2c3ccccc3n(-c3ccccc3)c2c1] | 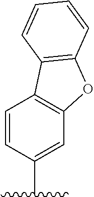 [SMILES: *c1ccc2c(c1)oc1ccccc12] |
| N35 | 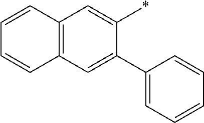 [SMILES: *c1cc2ccccc2cc1-c1ccccc1] | 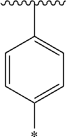 [SMILES: *c1ccc(*)cc1] | 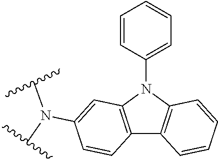 [SMILES: *N(*)c1ccc2c3ccccc3n(-c3ccccc3)c2c1] | 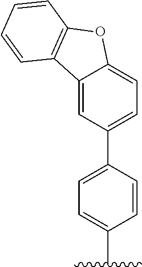 [SMILES: *c1ccc(-c2ccc3oc4ccccc4c3c2)cc1] |
| N36 | 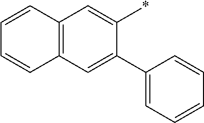 [SMILES: *c1cc2ccccc2cc1-c1ccccc1] | 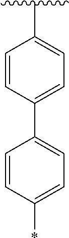 [SMILES: *c1ccc(-c2ccc(*)cc2)cc1] | 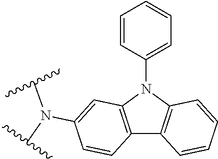 [SMILES: *N(*)c1ccc2c3ccccc3n(-c3ccccc3)c2c1] | 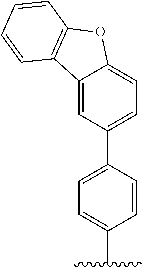 [SMILES: *c1ccc(-c2ccc3oc4ccccc4c3c2)cc1] |
| N37 | 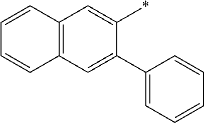 [SMILES: *c1cc2ccccc2cc1-c1ccccc1] | 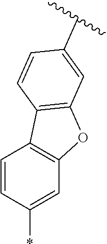 [SMILES: *c1ccc2c(c1)oc1cc(*)ccc12] | 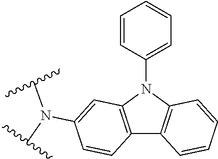 [SMILES: *N(*)c1ccc2c3ccccc3n(-c3ccccc3)c2c1] | 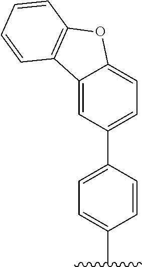 [SMILES: *c1ccc(-c2ccc3oc4ccccc4c3c2)cc1] |
| O1 | 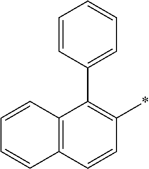 [SMILES: *c1ccc2ccccc2c1-c1ccccc1] | 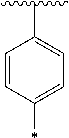 [SMILES: *c1ccc(*)cc1] | 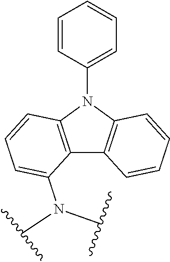 [SMILES: *N(*)c1cccc2c1c1ccccc1n2-c1ccccc1] | 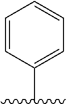 [SMILES: *c1ccccc1] |
| O2 | 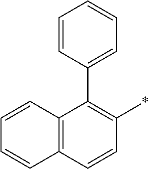 [SMILES: *c1ccc2ccccc2c1-c1ccccc1] | 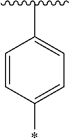 [SMILES: *c1ccc(*)cc1] | 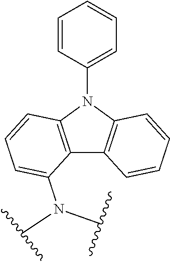 [SMILES: *N(*)c1cccc2c1c1ccccc1n2-c1ccccc1] | 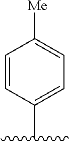 [SMILES: *c1ccc(C)cc1] |
| O3 | 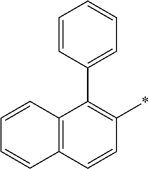 [SMILES: *c1ccc2ccccc2c1-c1ccccc1] | 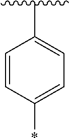 [SMILES: *c1ccc(*)cc1] | 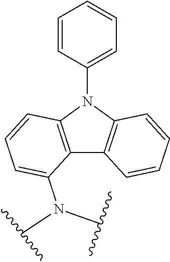 [SMILES: *N(*)c1cccc2c1c1ccccc1n2-c1ccccc1] | 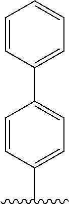 [SMILES: *c1ccc(-c2ccccc2)cc1] |
| O4 | 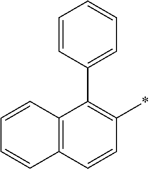 [SMILES: *c1ccc2ccccc2c1-c1ccccc1] | 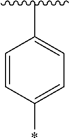 [SMILES: *c1ccc(*)cc1] | 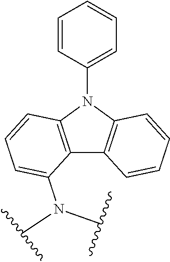 [SMILES: *N(*)c1cccc2c1c1ccccc1n2-c1ccccc1] | 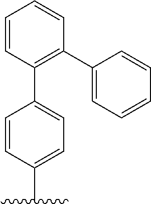 [SMILES: *c1ccc(-c2ccccc2-c2ccccc2)cc1] |
| O5 | 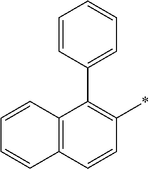 [SMILES: *c1ccc2ccccc2c1-c1ccccc1] | 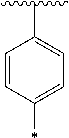 [SMILES: *c1ccc(*)cc1] | 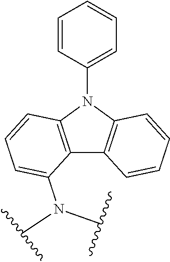 [SMILES: *N(*)c1cccc2c1c1ccccc1n2-c1ccccc1] | 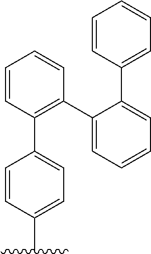 [SMILES: *c1ccc(-c2ccccc2-c2ccccc2-c2ccccc2)cc1] |
| O6 | 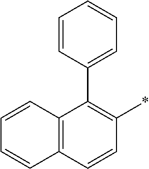 [SMILES: *c1ccc2ccccc2c1-c1ccccc1] | 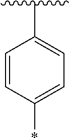 [SMILES: *c1ccc(*)cc1] | 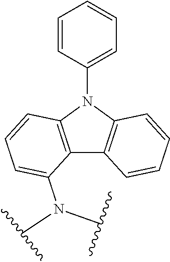 [SMILES: *N(*)c1cccc2c1c1ccccc1n2-c1ccccc1] | 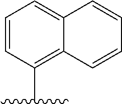 [SMILES: *c1cccc2ccccc12] |
| O7 | 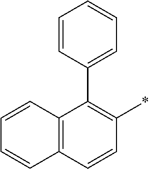 [SMILES: *c1ccc2ccccc2c1-c1ccccc1] | 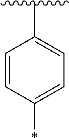 [SMILES: *c1ccc(*)cc1] | 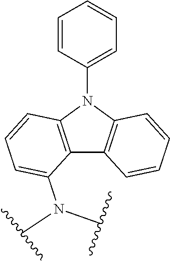 [SMILES: *N(*)c1cccc2c1c1ccccc1n2-c1ccccc1] | 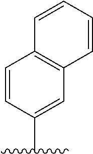 [SMILES: *c1ccc2ccccc2c1] |
| O8 | 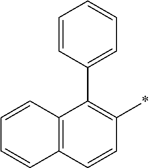 [SMILES: *c1ccc2ccccc2c1-c1ccccc1] | 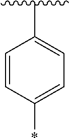 [SMILES: *c1ccc(*)cc1] | 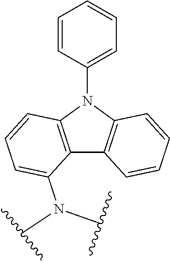 [SMILES: *N(*)c1cccc2c1c1ccccc1n2-c1ccccc1] | 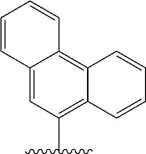 [SMILES: *c1cc2ccccc2c2ccccc12] |
| O9 | 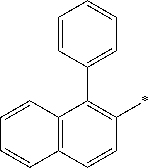 [SMILES: *c1ccc2ccccc2c1-c1ccccc1] | 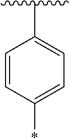 [SMILES: *c1ccc(*)cc1] | 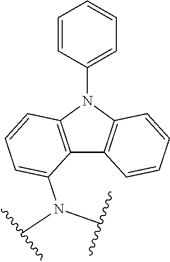 [SMILES: *N(*)c1cccc2c1c1ccccc1n2-c1ccccc1] | 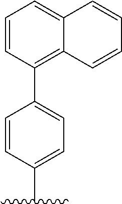 [SMILES: *c1ccc(-c2cccc3ccccc23)cc1] |
| O10 | 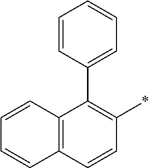 [SMILES: *c1ccc2ccccc2c1-c1ccccc1] | 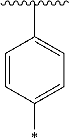 [SMILES: *c1ccc(*)cc1] | 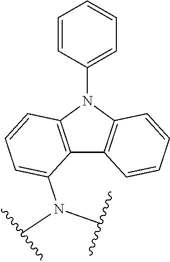 [SMILES: *N(*)c1cccc2c1c1ccccc1n2-c1ccccc1] | 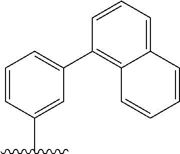 [SMILES: *c1cccc(-c2cccc3ccccc23)c1] |
| O11 | 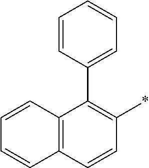 [SMILES: *c1ccc2ccccc2c1-c1ccccc1] | 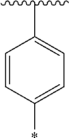 [SMILES: *c1ccc(*)cc1] | 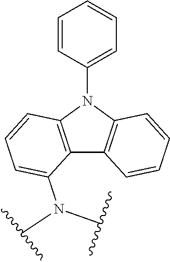 [SMILES: *N(*)c1cccc2c1c1ccccc1n2-c1ccccc1] | 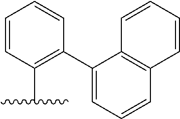 [SMILES: *c1ccccc1-c1cccc2ccccc12] |
| O12 | 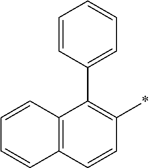 [SMILES: *c1ccc2ccccc2c1-c1ccccc1] | 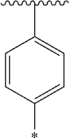 [SMILES: *c1ccc(*)cc1] | 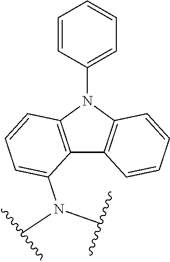 [SMILES: *N(*)c1cccc2c1c1ccccc1n2-c1ccccc1] | 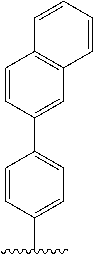 [SMILES: *c1ccc(-c2ccc3ccccc3c2)cc1] |
| O13 | 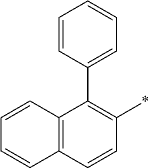 [SMILES: *c1ccc2ccccc2c1-c1ccccc1] | 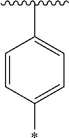 [SMILES: *c1ccc(*)cc1] | 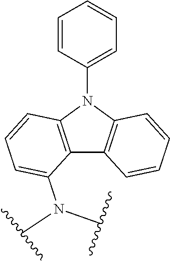 [SMILES: *N(*)c1cccc2c1c1ccccc1n2-c1ccccc1] | 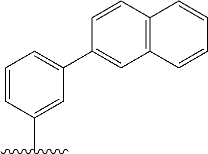 [SMILES: *c1cccc(-c2ccc3ccccc3c2)c1] |
| O14 | 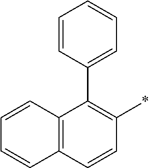 [SMILES: *c1ccc2ccccc2c1-c1ccccc1] | 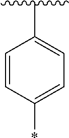 [SMILES: *c1ccc(*)cc1] | 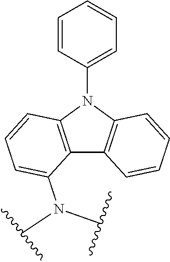 [SMILES: *N(*)c1cccc2c1c1ccccc1n2-c1ccccc1] | 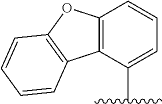 [SMILES: *c1cccc2oc3ccccc3c12] |
| O15 | 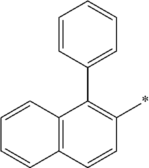 [SMILES: *c1ccc2ccccc2c1-c1ccccc1] | 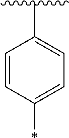 [SMILES: *c1ccc(*)cc1] | 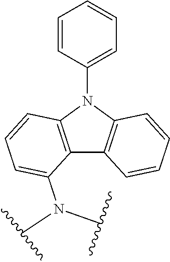 [SMILES: *N(*)c1cccc2c1c1ccccc1n2-c1ccccc1] | 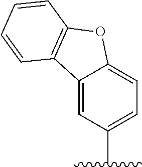 [SMILES: *c1ccc2oc3ccccc3c2c1] |
| O16 | 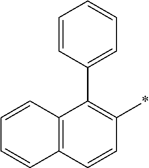 [SMILES: *c1ccc2ccccc2c1-c1ccccc1] | 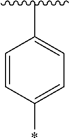 [SMILES: *c1ccc(*)cc1] | 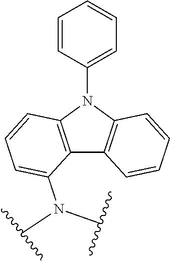 [SMILES: *N(*)c1cccc2c1c1ccccc1n2-c1ccccc1] | 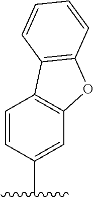 [SMILES: *c1ccc2c(c1)oc1ccccc12] |
| O17 | 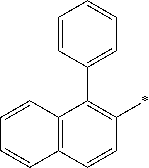 [SMILES: *c1ccc2ccccc2c1-c1ccccc1] | 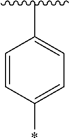 [SMILES: *c1ccc(*)cc1] | 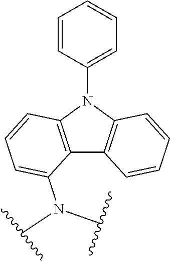 [SMILES: *N(*)c1cccc2c1c1ccccc1n2-c1ccccc1] | 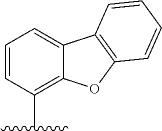 [SMILES: *c1cccc2c1oc1ccccc12] |
| O18 | 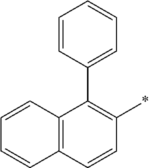 [SMILES: *c1ccc2ccccc2c1-c1ccccc1] | 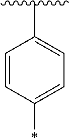 [SMILES: *c1ccc(*)cc1] | 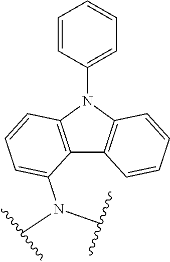 [SMILES: *N(*)c1cccc2c1c1ccccc1n2-c1ccccc1] | 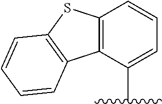 [SMILES: *c1cccc2sc3ccccc3c12] |
| O19 | 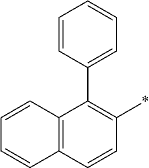 [SMILES: *c1ccc2ccccc2c1-c1ccccc1] | 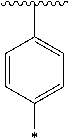 [SMILES: *c1ccc(*)cc1] | 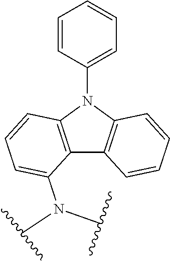 [SMILES: *N(*)c1cccc2c1c1ccccc1n2-c1ccccc1] | 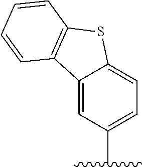 [SMILES: *c1ccc2sc3ccccc3c2c1] |
| O20 | 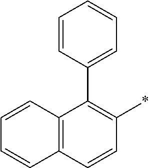 [SMILES: *c1ccc2ccccc2c1-c1ccccc1] | 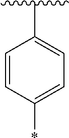 [SMILES: *c1ccc(*)cc1] | 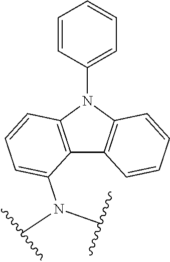 [SMILES: *N(*)c1cccc2c1c1ccccc1n2-c1ccccc1] | 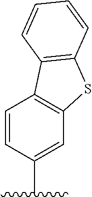 [SMILES: *c1ccc2c(c1)sc1ccccc12] |
| O21 | 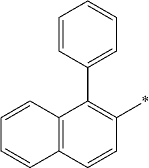 [SMILES: *c1ccc2ccccc2c1-c1ccccc1] | 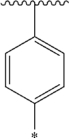 [SMILES: *c1ccc(*)cc1] | 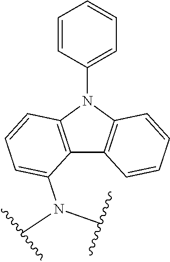 [SMILES: *N(*)c1cccc2c1c1ccccc1n2-c1ccccc1] | 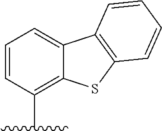 [SMILES: *c1cccc2c1sc1ccccc12] |
| O22 | 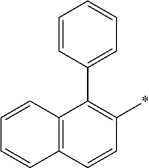 [SMILES: *c1ccc2ccccc2c1-c1ccccc1] | 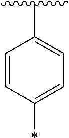 [SMILES: *c1ccc(*)cc1] | 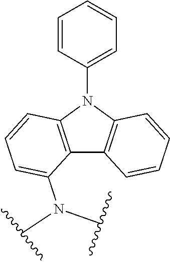 [SMILES: *N(*)c1cccc2c1c1ccccc1n2-c1ccccc1] | 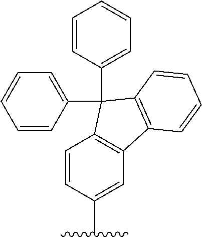 [SMILES: *c1ccc2c(c1)-c1ccccc1C2(c1ccccc1)c1ccccc1] |
| O23 | 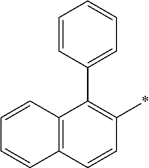 [SMILES: *c1ccc2ccccc2c1-c1ccccc1] | 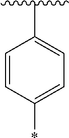 [SMILES: *c1ccc(*)cc1] | 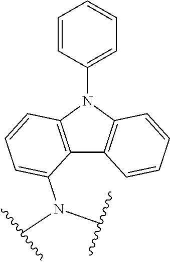 [SMILES: *N(*)c1cccc2c1c1ccccc1n2-c1ccccc1] | 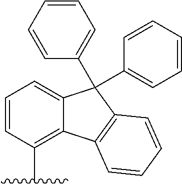 [SMILES: *c1cccc2c1-c1ccccc1C2(c1ccccc1)c1ccccc1] |
| O24 | 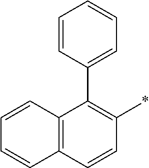 [SMILES: *c1ccc2ccccc2c1-c1ccccc1] | 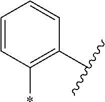 [SMILES: *c1ccccc1*] | 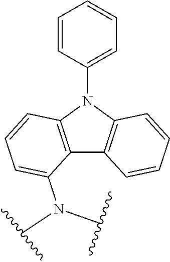 [SMILES: *N(*)c1cccc2c1c1ccccc1n2-c1ccccc1] | 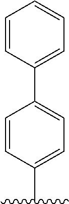 [SMILES: *c1ccc(-c2ccccc2)cc1] |
| O25 | 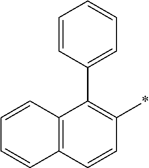 [SMILES: *c1ccc2ccccc2c1-c1ccccc1] | 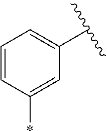 [SMILES: *c1cccc(*)c1] | 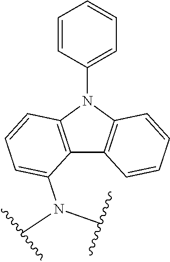 [SMILES: *N(*)c1cccc2c1c1ccccc1n2-c1ccccc1] | 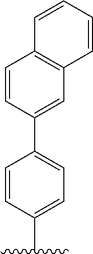 [SMILES: *c1ccc(-c2ccc3ccccc3c2)cc1] |
| O26 | 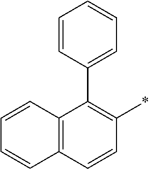 [SMILES: *c1ccc2ccccc2c1-c1ccccc1] | 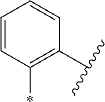 [SMILES: *c1ccccc1*] | 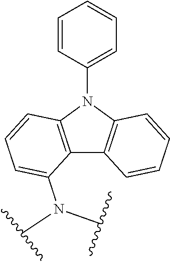 [SMILES: *N(*)c1cccc2c1c1ccccc1n2-c1ccccc1] | 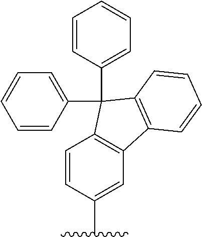 [SMILES: *c1ccc2c(c1)-c1ccccc1C2(c1ccccc1)c1ccccc1] |
| O27 | 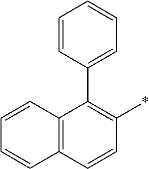 [SMILES: *c1ccc2ccccc2c1-c1ccccc1] | 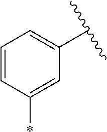 [SMILES: *c1cccc(*)c1] | 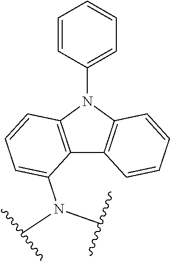 [SMILES: *N(*)c1cccc2c1c1ccccc1n2-c1ccccc1] | 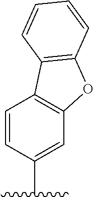 [SMILES: *c1ccc2c(c1)oc1ccccc12] |
| O28 | 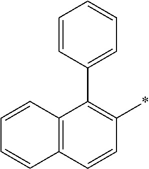 [SMILES: *c1ccc2ccccc2c1-c1ccccc1] | 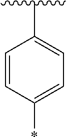 [SMILES: *c1ccc(*)cc1] | 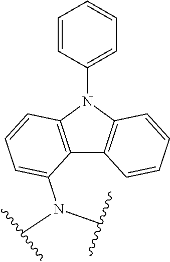 [SMILES: *N(*)c1cccc2c1c1ccccc1n2-c1ccccc1] | 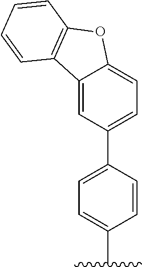 [SMILES: *c1ccc(-c2ccc3oc4ccccc4c3c2)cc1] |
| O29 | 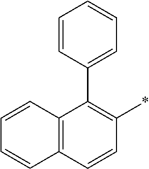 [SMILES: *c1ccc2ccccc2c1-c1ccccc1] | 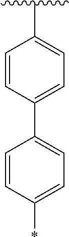 [SMILES: *c1ccc(-c2ccc(*)cc2)cc1] | 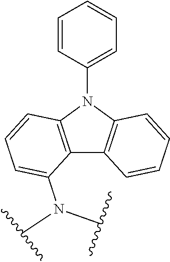 [SMILES: *N(*)c1cccc2c1c1ccccc1n2-c1ccccc1] | 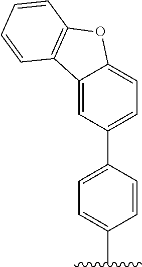 [SMILES: *c1ccc(-c2ccc3oc4ccccc4c3c2)cc1] |
| O30 | 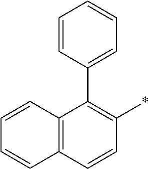 [SMILES: *c1ccc2ccccc2c1-c1ccccc1] | 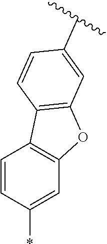 [SMILES: *c1ccc2c(c1)oc1cc(*)ccc12] | 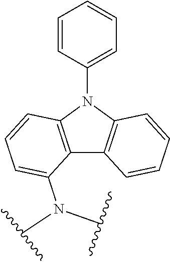 [SMILES: *N(*)c1cccc2c1c1ccccc1n2-c1ccccc1] | 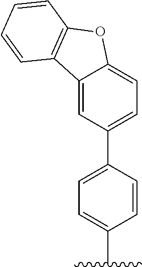 [SMILES: *c1ccc(-c2ccc3oc4ccccc4c3c2)cc1] |
| O31 | 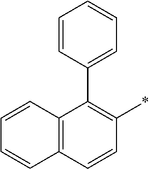 [SMILES: *c1ccc2ccccc2c1-c1ccccc1] | 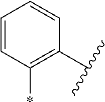 [SMILES: *c1ccccc1*] | 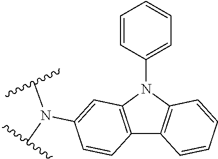 [SMILES: *N(*)c1ccc2c3ccccc3n(-c3ccccc3)c2c1] | 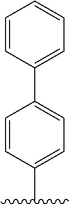 [SMILES: *c1ccc(-c2ccccc2)cc1] |
| O32 | 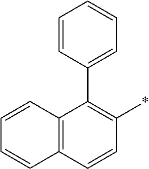 [SMILES: *c1ccc2ccccc2c1-c1ccccc1] | 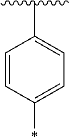 [SMILES: *c1ccc(*)cc1] | 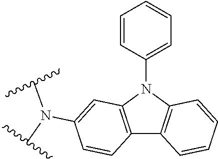 [SMILES: *N(*)c1ccc2c3ccccc3n(-c3ccccc3)c2c1] | 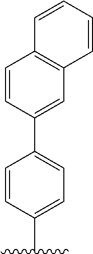 [SMILES: *c1ccc(-c2ccc3ccccc3c2)cc1] |
| O33 | 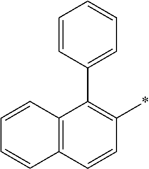 [SMILES: *c1ccc2ccccc2c1-c1ccccc1] | 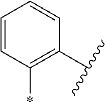 [SMILES: *c1ccccc1*] | 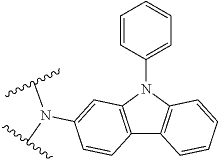 [SMILES: *N(*)c1ccc2c3ccccc3n(-c3ccccc3)c2c1] | 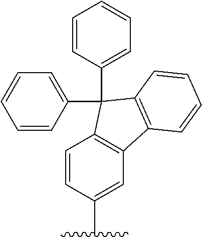 [SMILES: *c1ccc2c(c1)-c1ccccc1C2(c1ccccc1)c1ccccc1] |
| O34 | 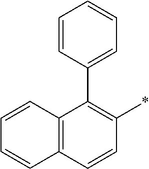 [SMILES: *c1ccc2ccccc2c1-c1ccccc1] | 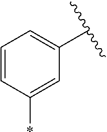 [SMILES: *c1cccc(*)c1] | 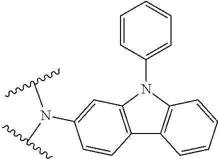 [SMILES: *N(*)c1ccc2c3ccccc3n(-c3ccccc3)c2c1] | 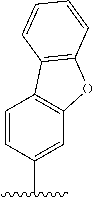 [SMILES: *c1ccc2c(c1)oc1ccccc12] |
| O35 | 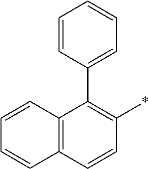 [SMILES: *c1ccc2ccccc2c1-c1ccccc1] | 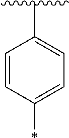 [SMILES: *c1ccc(*)cc1] | 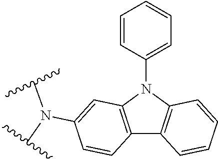 [SMILES: *N(*)c1ccc2c3ccccc3n(-c3ccccc3)c2c1] | 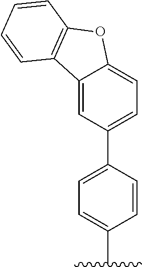 [SMILES: *c1ccc(-c2ccc3oc4ccccc4c3c2)cc1] |
| O36 | 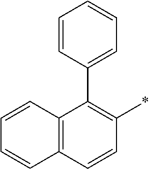 [SMILES: *c1ccc2ccccc2c1-c1ccccc1] | 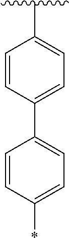 [SMILES: *c1ccc(-c2ccc(*)cc2)cc1] | 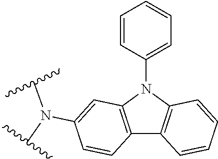 [SMILES: *N(*)c1ccc2c3ccccc3n(-c3ccccc3)c2c1] | 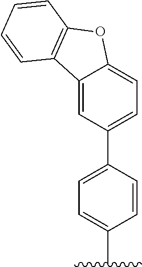 [SMILES: *c1ccc(-c2ccc3oc4ccccc4c3c2)cc1] |
| O37 | 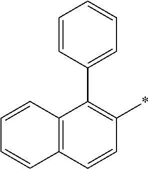 [SMILES: *c1ccc2ccccc2c1-c1ccccc1] | 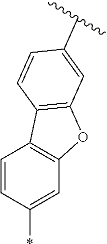 [SMILES: *c1ccc2c(c1)oc1cc(*)ccc12] | 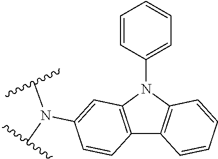 [SMILES: *N(*)c1ccc2c3ccccc3n(-c3ccccc3)c2c1] | 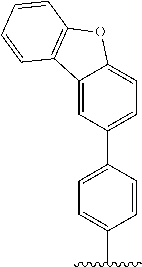 [SMILES: *c1ccc(-c2ccc3oc4ccccc4c3c2)cc1] |
| P1 | 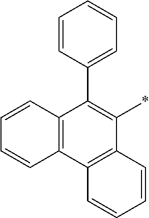 [SMILES: *c1c(-c2ccccc2)c2ccccc2c2ccccc12] | 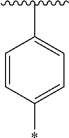 [SMILES: *c1ccc(*)cc1] | 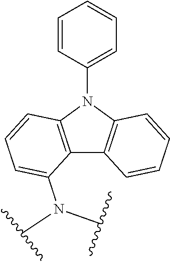 [SMILES: *N(*)c1cccc2c1c1ccccc1n2-c1ccccc1] | 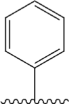 [SMILES: *c1ccccc1] |
| P2 | 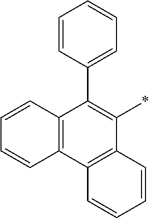 [SMILES: *c1c(-c2ccccc2)c2ccccc2c2ccccc12] | 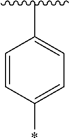 [SMILES: *c1ccc(*)cc1] | 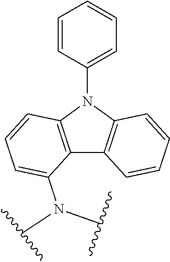 [SMILES: *N(*)c1cccc2c1c1ccccc1n2-c1ccccc1] | 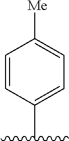 [SMILES: *c1ccc(C)cc1] |
| P3 | 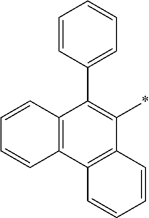 [SMILES: *c1c(-c2ccccc2)c2ccccc2c2ccccc12] | 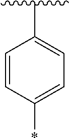 [SMILES: *c1ccc(*)cc1] | 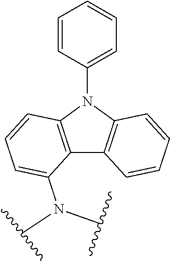 [SMILES: *N(*)c1cccc2c1c1ccccc1n2-c1ccccc1] | 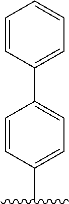 [SMILES: *c1ccc(-c2ccccc2)cc1] |
| P4 | 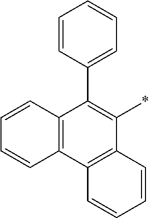 [SMILES: *c1c(-c2ccccc2)c2ccccc2c2ccccc12] | 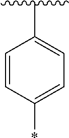 [SMILES: *c1ccc(*)cc1] | 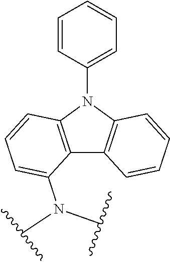 [SMILES: *N(*)c1cccc2c1c1ccccc1n2-c1ccccc1] | 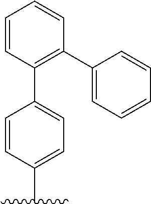 [SMILES: *c1ccc(-c2ccccc2-c2ccccc2)cc1] |
| P5 | 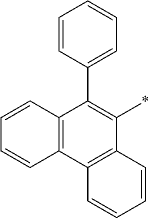 [SMILES: *c1c(-c2ccccc2)c2ccccc2c2ccccc12] | 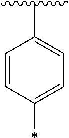 [SMILES: *c1ccc(*)cc1] | 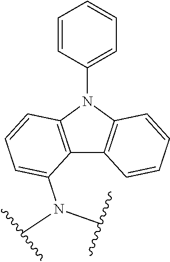 [SMILES: *N(*)c1cccc2c1c1ccccc1n2-c1ccccc1] | 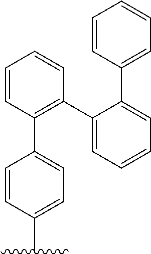 [SMILES: *c1ccc(-c2ccccc2-c2ccccc2-c2ccccc2)cc1] |
| P6 | 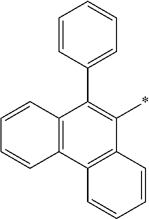 [SMILES: *c1c(-c2ccccc2)c2ccccc2c2ccccc12] | 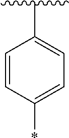 [SMILES: *c1ccc(*)cc1] | 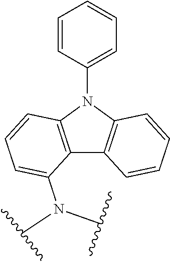 [SMILES: *N(*)c1cccc2c1c1ccccc1n2-c1ccccc1] | 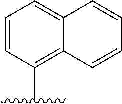 [SMILES: *c1cccc2ccccc12] |
| P7 | 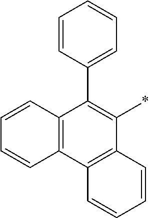 [SMILES: *c1c(-c2ccccc2)c2ccccc2c2ccccc12] | 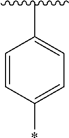 [SMILES: *c1ccc(*)cc1] | 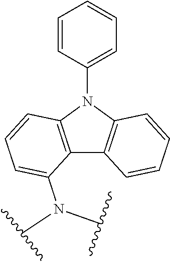 [SMILES: *N(*)c1cccc2c1c1ccccc1n2-c1ccccc1] | 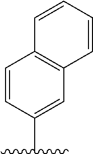 [SMILES: *c1ccc2ccccc2c1] |
| P8 | 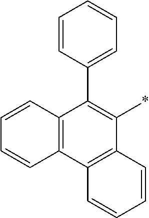 [SMILES: *c1c(-c2ccccc2)c2ccccc2c2ccccc12] | 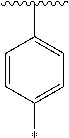 [SMILES: *c1ccc(*)cc1] | 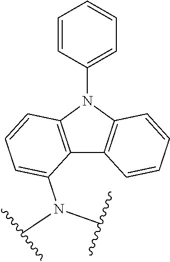 [SMILES: *N(*)c1cccc2c1c1ccccc1n2-c1ccccc1] | 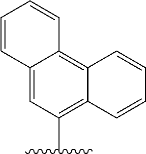 [SMILES: *c1cc2ccccc2c2ccccc12] |
| P9 | 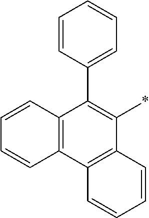 [SMILES: *c1c(-c2ccccc2)c2ccccc2c2ccccc12] | 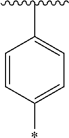 [SMILES: *c1ccc(*)cc1] | 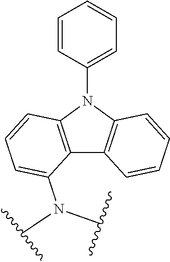 [SMILES: *N(*)c1cccc2c1c1ccccc1n2-c1ccccc1] | 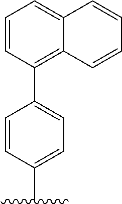 [SMILES: *c1ccc(-c2cccc3ccccc23)cc1] |
| P10 | 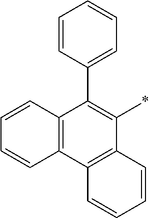 [SMILES: *c1c(-c2ccccc2)c2ccccc2c2ccccc12] | 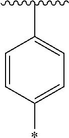 [SMILES: *c1ccc(*)cc1] | 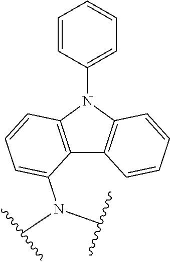 [SMILES: *N(*)c1cccc2c1c1ccccc1n2-c1ccccc1] | 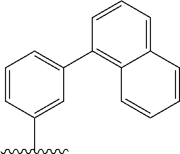 [SMILES: *c1cccc(-c2cccc3ccccc23)c1] |
| P11 | 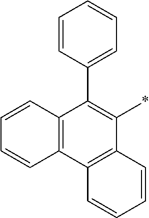 [SMILES: *c1c(-c2ccccc2)c2ccccc2c2ccccc12] | 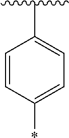 [SMILES: *c1ccc(*)cc1] | 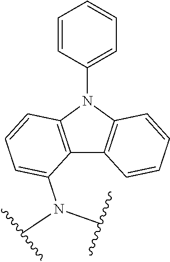 [SMILES: *N(*)c1cccc2c1c1ccccc1n2-c1ccccc1] | 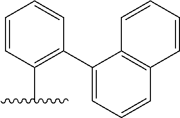 [SMILES: *c1ccccc1-c1cccc2ccccc12] |
| P12 | 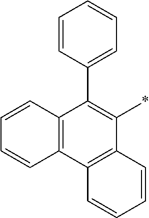 [SMILES: *c1c(-c2ccccc2)c2ccccc2c2ccccc12] | 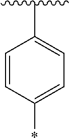 [SMILES: *c1ccc(*)cc1] | 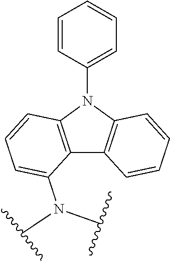 [SMILES: *N(*)c1cccc2c1c1ccccc1n2-c1ccccc1] | 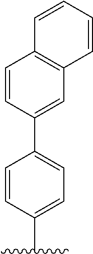 [SMILES: *c1ccc(-c2ccc3ccccc3c2)cc1] |
| P13 | 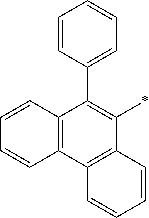 [SMILES: *c1c(-c2ccccc2)c2ccccc2c2ccccc12] | 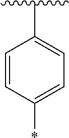 [SMILES: *c1ccc(*)cc1] | 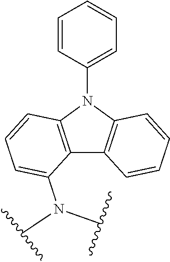 [SMILES: *N(*)c1cccc2c1c1ccccc1n2-c1ccccc1] | 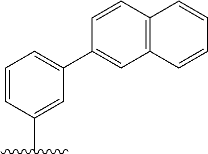 [SMILES: *c1cccc(-c2ccc3ccccc3c2)c1] |
| P14 | 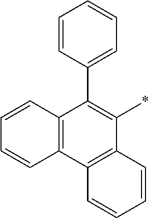 [SMILES: *c1c(-c2ccccc2)c2ccccc2c2ccccc12] | 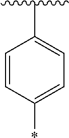 [SMILES: *c1ccc(*)cc1] | 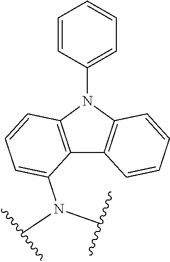 [SMILES: *N(*)c1cccc2c1c1ccccc1n2-c1ccccc1] | 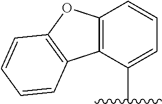 [SMILES: *c1cccc2oc3ccccc3c12] |
| P15 | 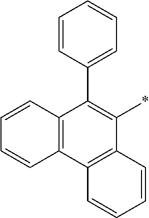 [SMILES: *c1c(-c2ccccc2)c2ccccc2c2ccccc12] | 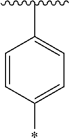 [SMILES: *c1ccc(*)cc1] | 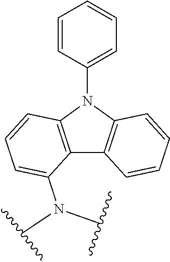 [SMILES: *N(*)c1cccc2c1c1ccccc1n2-c1ccccc1] | 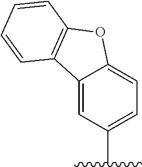 [SMILES: *c1ccc2oc3ccccc3c2c1] |
| P16 | 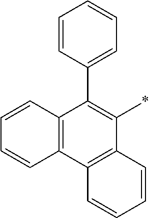 [SMILES: *c1c(-c2ccccc2)c2ccccc2c2ccccc12] | 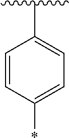 [SMILES: *c1ccc(*)cc1] | 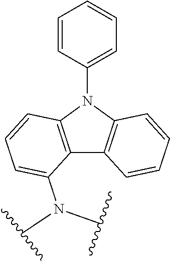 [SMILES: *N(*)c1cccc2c1c1ccccc1n2-c1ccccc1] | 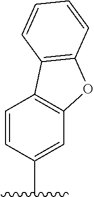 [SMILES: *c1ccc2c(c1)oc1ccccc12] |
| P17 | 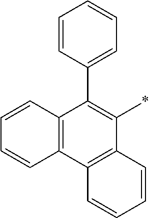 [SMILES: *c1c(-c2ccccc2)c2ccccc2c2ccccc12] | 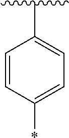 [SMILES: *c1ccc(*)cc1] | 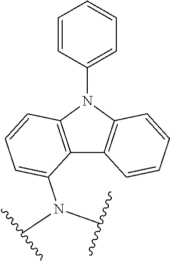 [SMILES: *N(*)c1cccc2c1c1ccccc1n2-c1ccccc1] | 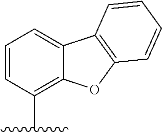 [SMILES: *c1cccc2c1oc1ccccc12] |
| P18 | 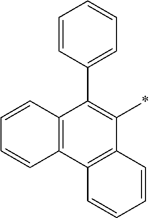 [SMILES: *c1c(-c2ccccc2)c2ccccc2c2ccccc12] | 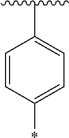 [SMILES: *c1ccc(*)cc1] | 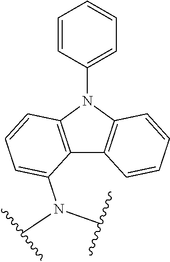 [SMILES: *N(*)c1cccc2c1c1ccccc1n2-c1ccccc1] | 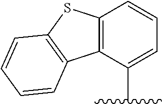 [SMILES: *c1cccc2sc3ccccc3c12] |
| P19 | 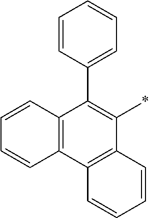 [SMILES: *c1c(-c2ccccc2)c2ccccc2c2ccccc12] | 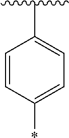 [SMILES: *c1ccc(*)cc1] | 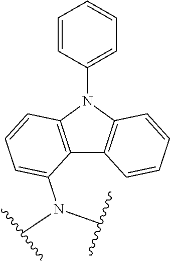 [SMILES: *N(*)c1cccc2c1c1ccccc1n2-c1ccccc1] | 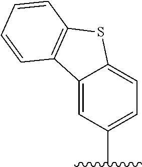 [SMILES: *c1ccc2sc3ccccc3c2c1] |
| P20 | 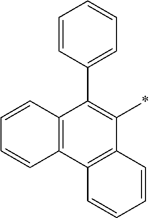 [SMILES: *c1c(-c2ccccc2)c2ccccc2c2ccccc12] | 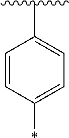 [SMILES: *c1ccc(*)cc1] | 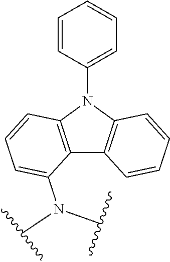 [SMILES: *N(*)c1cccc2c1c1ccccc1n2-c1ccccc1] | 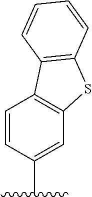 [SMILES: *c1ccc2c(c1)sc1ccccc12] |
| P21 | 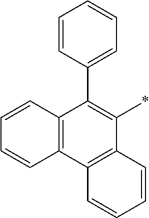 [SMILES: *c1c(-c2ccccc2)c2ccccc2c2ccccc12] | 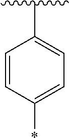 [SMILES: *c1ccc(*)cc1] | 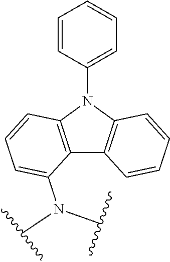 [SMILES: *N(*)c1cccc2c1c1ccccc1n2-c1ccccc1] | 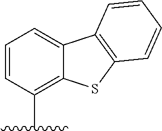 [SMILES: *c1cccc2c1sc1ccccc12] |
| P22 | 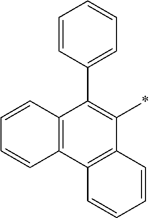 [SMILES: *c1c(-c2ccccc2)c2ccccc2c2ccccc12] | 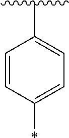 [SMILES: *c1ccc(*)cc1] | 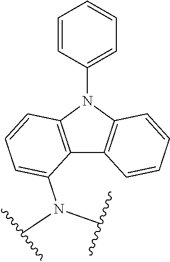 [SMILES: *N(*)c1cccc2c1c1ccccc1n2-c1ccccc1] | 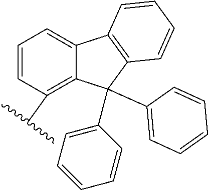 [SMILES: *c1cccc2c1C(c1ccccc1)(c1ccccc1)c1ccccc1-2] |
| P23 | 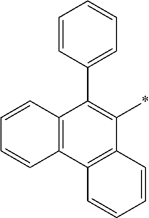 [SMILES: *c1c(-c2ccccc2)c2ccccc2c2ccccc12] | 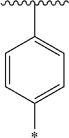 [SMILES: *c1ccc(*)cc1] | 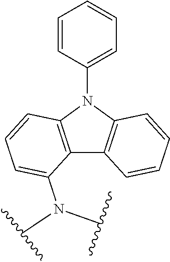 [SMILES: *N(*)c1cccc2c1c1ccccc1n2-c1ccccc1] | 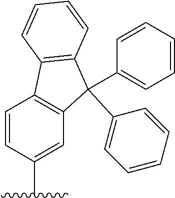 [SMILES: *c1ccc2c(c1)C(c1ccccc1)(c1ccccc1)c1ccccc1-2] |
| P24 | 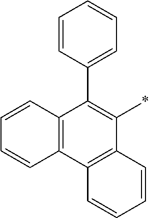 [SMILES: *c1c(-c2ccccc2)c2ccccc2c2ccccc12] | 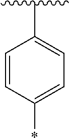 [SMILES: *c1ccc(*)cc1] | 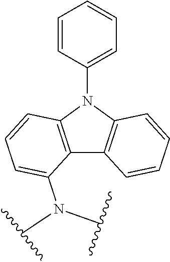 [SMILES: *N(*)c1cccc2c1c1ccccc1n2-c1ccccc1] | 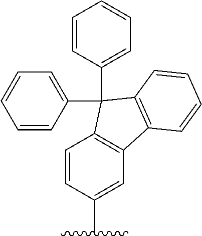 [SMILES: *c1ccc2c(c1)-c1ccccc1C2(c1ccccc1)c1ccccc1] |
| P25 | 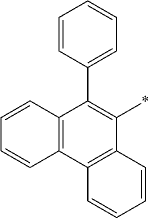 [SMILES: *c1c(-c2ccccc2)c2ccccc2c2ccccc12] | 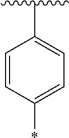 [SMILES: *c1ccc(*)cc1] | 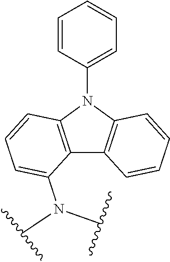 [SMILES: *N(*)c1cccc2c1c1ccccc1n2-c1ccccc1] | 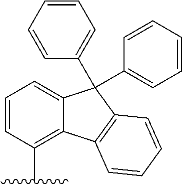 [SMILES: *c1cccc2c1-c1ccccc1C2(c1ccccc1)c1ccccc1] |
| P26 | 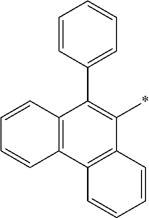 [SMILES: *c1c(-c2ccccc2)c2ccccc2c2ccccc12] | 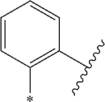 [SMILES: *c1ccccc1*] | 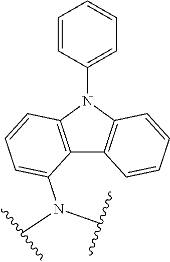 [SMILES: *N(*)c1cccc2c1c1ccccc1n2-c1ccccc1] | 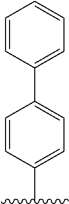 [SMILES: *c1ccc(-c2ccccc2)cc1] |
| P27 | 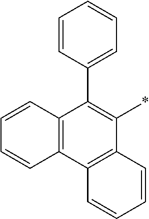 [SMILES: *c1c(-c2ccccc2)c2ccccc2c2ccccc12] | 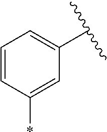 [SMILES: *c1cccc(*)c1] | 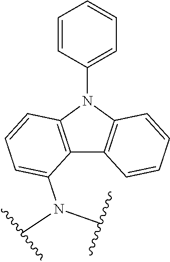 [SMILES: *N(*)c1cccc2c1c1ccccc1n2-c1ccccc1] | 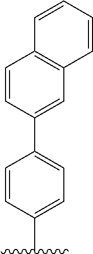 [SMILES: *c1ccc(-c2ccc3ccccc3c2)cc1] |
| P28 | 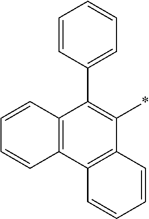 [SMILES: *c1c(-c2ccccc2)c2ccccc2c2ccccc12] | 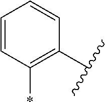 [SMILES: *c1ccccc1*] | 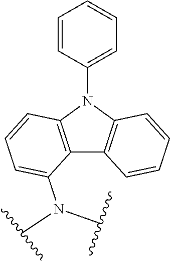 [SMILES: *N(*)c1cccc2c1c1ccccc1n2-c1ccccc1] | 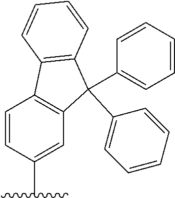 [SMILES: *c1ccc2c(c1)C(c1ccccc1)(c1ccccc1)c1ccccc1-2] |
| P29 | 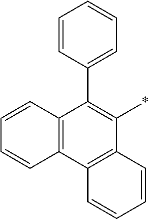 [SMILES: *c1c(-c2ccccc2)c2ccccc2c2ccccc12] | 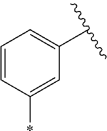 [SMILES: *c1cccc(*)c1] | 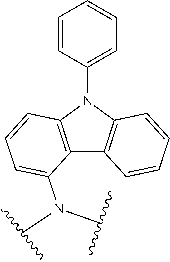 [SMILES: *N(*)c1cccc2c1c1ccccc1n2-c1ccccc1] | 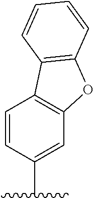 [SMILES: *c1ccc2c(c1)oc1ccccc12] |
| P30 | 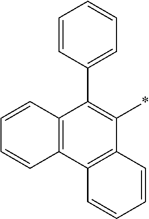 [SMILES: *c1c(-c2ccccc2)c2ccccc2c2ccccc12] | 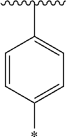 [SMILES: *c1ccc(*)cc1] | 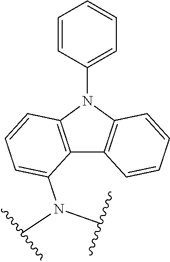 [SMILES: *N(*)c1cccc2c1c1ccccc1n2-c1ccccc1] | 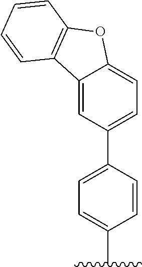 [SMILES: *c1ccc(-c2ccc3oc4ccccc4c3c2)cc1] |
| P31 | 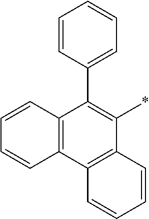 [SMILES: *c1c(-c2ccccc2)c2ccccc2c2ccccc12] | 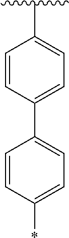 [SMILES: *c1ccc(-c2ccc(*)cc2)cc1] | 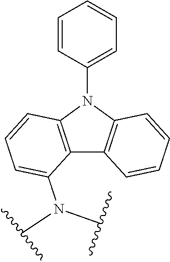 [SMILES: *N(*)c1cccc2c1c1ccccc1n2-c1ccccc1] | 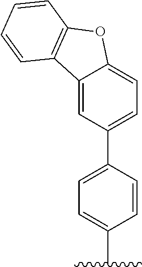 [SMILES: *c1ccc(-c2ccc3oc4ccccc4c3c2)cc1] |
| P32 | 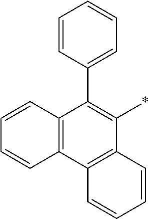 [SMILES: *c1c(-c2ccccc2)c2ccccc2c2ccccc12] | 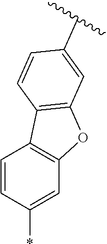 [SMILES: *c1ccc2c(c1)oc1cc(*)ccc12] | 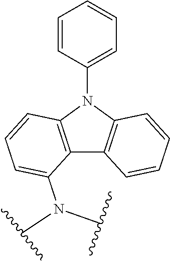 [SMILES: *N(*)c1cccc2c1c1ccccc1n2-c1ccccc1] | 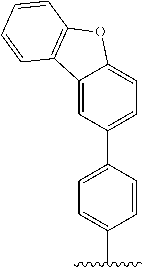 [SMILES: *c1ccc(-c2ccc3oc4ccccc4c3c2)cc1] |
| P33 | 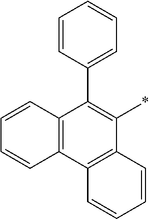 [SMILES: *c1c(-c2ccccc2)c2ccccc2c2ccccc12] | 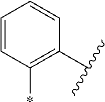 [SMILES: *c1ccccc1*] | 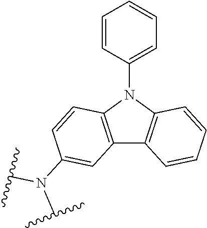 [SMILES: *N(*)c1ccc2c(c1)c1ccccc1n2-c1ccccc1] | 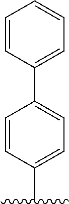 [SMILES: *c1ccc(-c2ccccc2)cc1] |
| P34 | 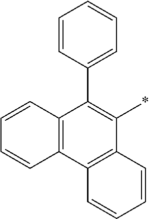 [SMILES: *c1c(-c2ccccc2)c2ccccc2c2ccccc12] | 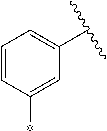 [SMILES: *c1cccc(*)c1] | 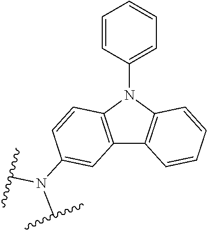 [SMILES: *N(*)c1ccc2c(c1)c1ccccc1n2-c1ccccc1] | 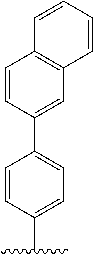 [SMILES: *c1ccc(-c2ccc3ccccc3c2)cc1] |
| P35 | 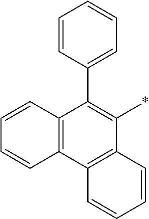 [SMILES: *c1c(-c2ccccc2)c2ccccc2c2ccccc12] | 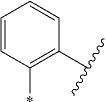 [SMILES: *c1ccccc1*] | 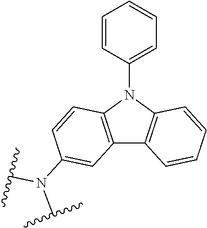 [SMILES: *N(*)c1ccc2c(c1)c1ccccc1n2-c1ccccc1] | 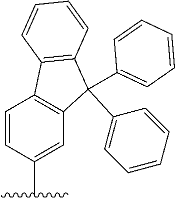 [SMILES: *c1ccc2c(c1)C(c1ccccc1)(c1ccccc1)c1ccccc1-2] |
| P36 | 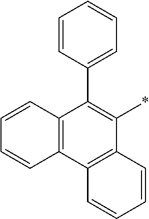 [SMILES: *c1c(-c2ccccc2)c2ccccc2c2ccccc12] | 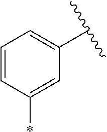 [SMILES: *c1cccc(*)c1] | 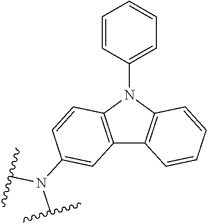 [SMILES: *N(*)c1ccc2c(c1)c1ccccc1n2-c1ccccc1] | 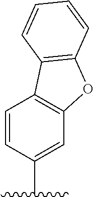 [SMILES: *c1ccc2c(c1)oc1ccccc12] |
| P37 | 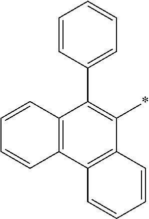 [SMILES: *c1c(-c2ccccc2)c2ccccc2c2ccccc12] | 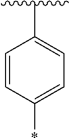 [SMILES: *c1ccc(*)cc1] | 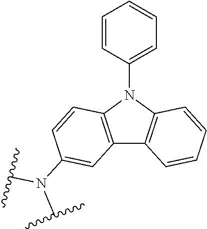 [SMILES: *N(*)c1ccc2c(c1)c1ccccc1n2-c1ccccc1] | 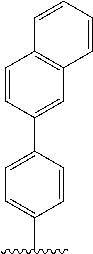 [SMILES: *c1ccc(-c2ccc3ccccc3c2)cc1] |
| P38 | 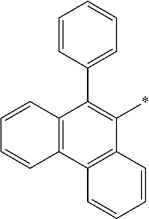 [SMILES: *c1c(-c2ccccc2)c2ccccc2c2ccccc12] | 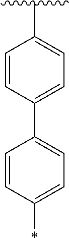 [SMILES: *c1ccc(-c2ccc(*)cc2)cc1] | 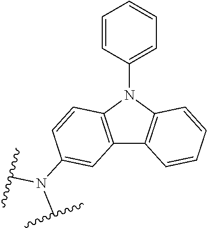 [SMILES: *N(*)c1ccc2c(c1)c1ccccc1n2-c1ccccc1] | 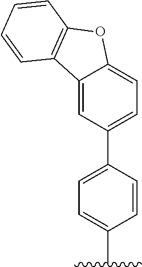 [SMILES: *c1ccc(-c2ccc3oc4ccccc4c3c2)cc1] |
| P39 | 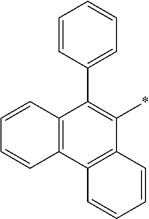 [SMILES: *c1c(-c2ccccc2)c2ccccc2c2ccccc12] | 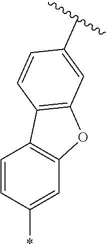 [SMILES: *c1ccc2c(c1)oc1cc(*)ccc12] | 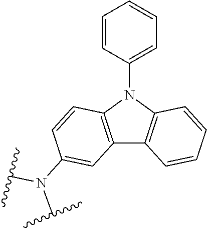 [SMILES: *N(*)c1ccc2c(c1)c1ccccc1n2-c1ccccc1] | 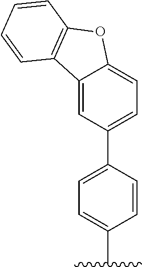 [SMILES: *c1ccc(-c2ccc3oc4ccccc4c3c2)cc1] |
| P40 | 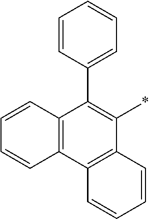 [SMILES: *c1c(-c2ccccc2)c2ccccc2c2ccccc12] | 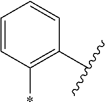 [SMILES: *c1ccccc1*] | 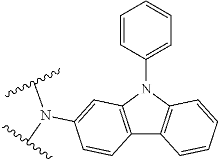 [SMILES: *N(*)c1ccc2c3ccccc3n(-c3ccccc3)c2c1] | 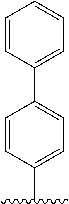 [SMILES: *c1ccc(-c2ccccc2)cc1] |
| P41 | 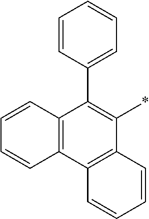 [SMILES: *c1c(-c2ccccc2)c2ccccc2c2ccccc12] | 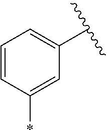 [SMILES: *c1cccc(*)c1] | 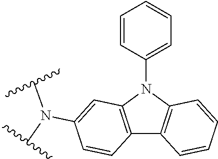 [SMILES: *N(*)c1ccc2c3ccccc3n(-c3ccccc3)c2c1] | 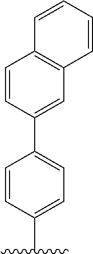 [SMILES: *c1ccc(-c2ccc3ccccc3c2)cc1] |
| P42 | 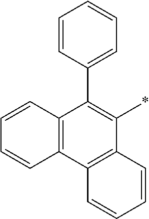 [SMILES: *c1c(-c2ccccc2)c2ccccc2c2ccccc12] | 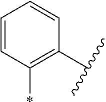 [SMILES: *c1ccccc1*] | 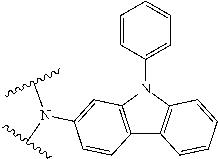 [SMILES: *N(*)c1ccc2c3ccccc3n(-c3ccccc3)c2c1] | 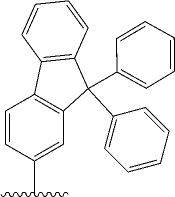 [SMILES: *c1ccc2c(c1)C(c1ccccc1)(c1ccccc1)c1ccccc1-2] |
| P43 | 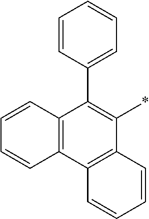 [SMILES: *c1c(-c2ccccc2)c2ccccc2c2ccccc12] | 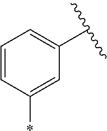 [SMILES: *c1cccc(*)c1] | 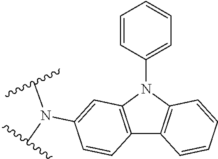 [SMILES: *N(*)c1ccc2c3ccccc3n(-c3ccccc3)c2c1] | 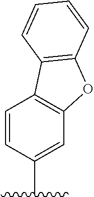 [SMILES: *c1ccc2c(c1)oc1ccccc12] |
| P44 | 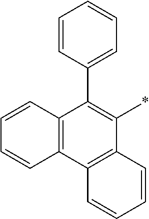 [SMILES: *c1c(-c2ccccc2)c2ccccc2c2ccccc12] | 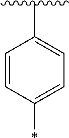 [SMILES: *c1ccc(*)cc1] | 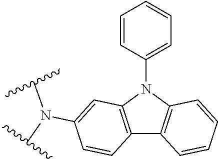 [SMILES: *N(*)c1ccc2c3ccccc3n(-c3ccccc3)c2c1] | 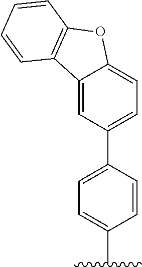 [SMILES: *c1ccc(-c2ccc3oc4ccccc4c3c2)cc1] |
| P45 | 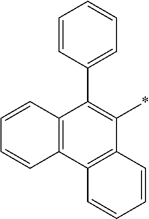 [SMILES: *c1c(-c2ccccc2)c2ccccc2c2ccccc12] | 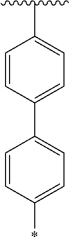 [SMILES: *c1ccc(-c2ccc(*)cc2)cc1] | 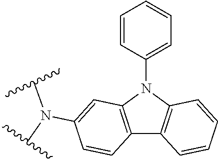 [SMILES: *N(*)c1ccc2c3ccccc3n(-c3ccccc3)c2c1] | 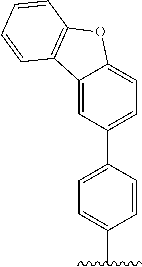 [SMILES: *c1ccc(-c2ccc3oc4ccccc4c3c2)cc1] |
| P46 | 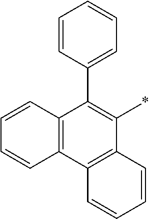 [SMILES: *c1c(-c2ccccc2)c2ccccc2c2ccccc12] | 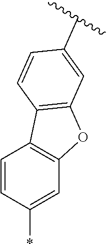 [SMILES: *c1ccc2c(c1)oc1cc(*)ccc12] | 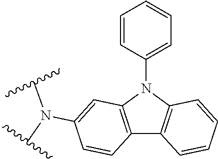 [SMILES: *N(*)c1ccc2c3ccccc3n(-c3ccccc3)c2c1] | 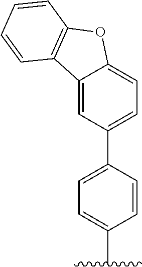 [SMILES: *c1ccc(-c2ccc3oc4ccccc4c3c2)cc1] |
| Q1 | 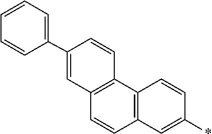 [SMILES: *c1ccc2c(ccc3cc(-c4ccccc4)ccc32)c1] | 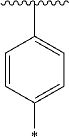 [SMILES: *c1ccc(*)cc1] | 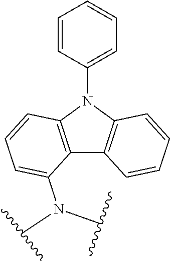 [SMILES: *N(*)c1cccc2c1c1ccccc1n2-c1ccccc1] | 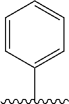 [SMILES: *c1ccccc1] |
| Q2 | 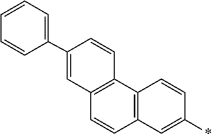 [SMILES: *c1ccc2c(ccc3cc(-c4ccccc4)ccc32)c1] | 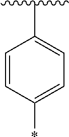 [SMILES: *c1ccc(*)cc1] | 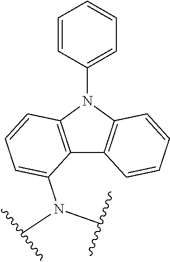 [SMILES: *N(*)c1cccc2c1c1ccccc1n2-c1ccccc1] | 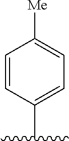 [SMILES: *c1ccc(C)cc1] |
| Q3 | 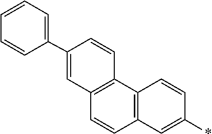 [SMILES: *c1ccc2c(ccc3cc(-c4ccccc4)ccc32)c1] | 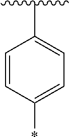 [SMILES: *c1ccc(*)cc1] | 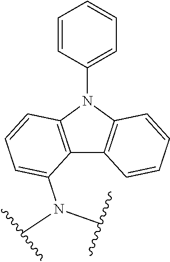 [SMILES: *N(*)c1cccc2c1c1ccccc1n2-c1ccccc1] | 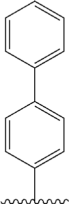 [SMILES: *c1ccc(-c2ccccc2)cc1] |
| Q4 | 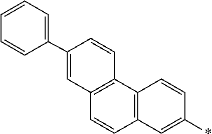 [SMILES: *c1ccc2c(ccc3cc(-c4ccccc4)ccc32)c1] | 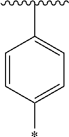 [SMILES: *c1ccc(*)cc1] | 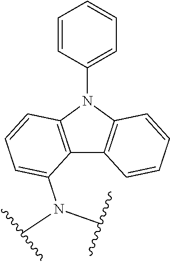 [SMILES: *N(*)c1cccc2c1c1ccccc1n2-c1ccccc1] | 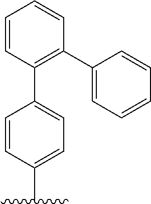 [SMILES: *c1ccc(-c2ccccc2-c2ccccc2)cc1] |
| Q5 | 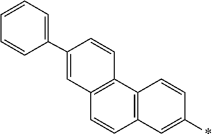 [SMILES: *c1ccc2c(ccc3cc(-c4ccccc4)ccc32)c1] | 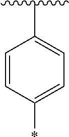 [SMILES: *c1ccc(*)cc1] | 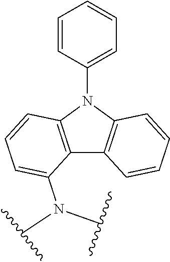 [SMILES: *N(*)c1cccc2c1c1ccccc1n2-c1ccccc1] | 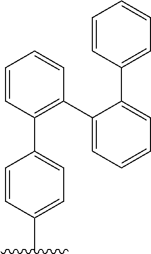 [SMILES: *c1ccc(-c2ccccc2-c2ccccc2-c2ccccc2)cc1] |
| Q6 | 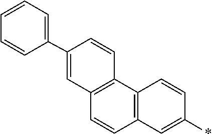 [SMILES: *c1ccc2c(ccc3cc(-c4ccccc4)ccc32)c1] | 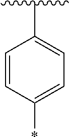 [SMILES: *c1ccc(*)cc1] | 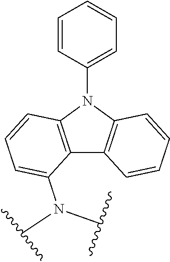 [SMILES: *N(*)c1cccc2c1c1ccccc1n2-c1ccccc1] | 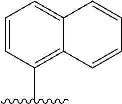 [SMILES: *c1cccc2ccccc12] |
| Q7 | 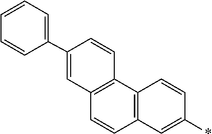 [SMILES: *c1ccc2c(ccc3cc(-c4ccccc4)ccc32)c1] | 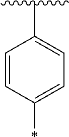 [SMILES: *c1ccc(*)cc1] | 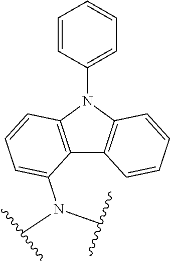 [SMILES: *N(*)c1cccc2c1c1ccccc1n2-c1ccccc1] | 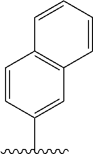 [SMILES: *c1ccc2ccccc2c1] |
| Q8 | 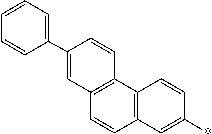 [SMILES: *c1ccc2c(ccc3cc(-c4ccccc4)ccc32)c1] | 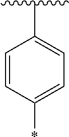 [SMILES: *c1ccc(*)cc1] | 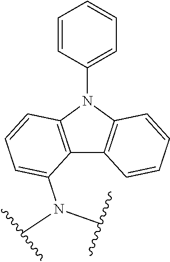 [SMILES: *N(*)c1cccc2c1c1ccccc1n2-c1ccccc1] | 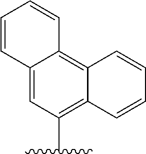 [SMILES: *c1cc2ccccc2c2ccccc12] |
| Q9 | 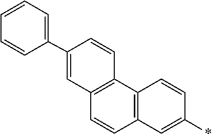 [SMILES: *c1ccc2c(ccc3cc(-c4ccccc4)ccc32)c1] | 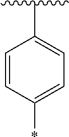 [SMILES: *c1ccc(*)cc1] | 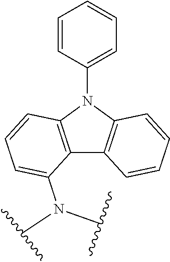 [SMILES: *N(*)c1cccc2c1c1ccccc1n2-c1ccccc1] | 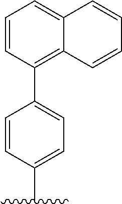 [SMILES: *c1ccc(-c2cccc3ccccc23)cc1] |
| Q10 | 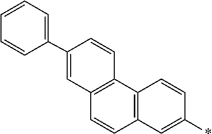 [SMILES: *c1ccc2c(ccc3cc(-c4ccccc4)ccc32)c1] | 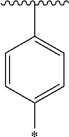 [SMILES: *c1ccc(*)cc1] | 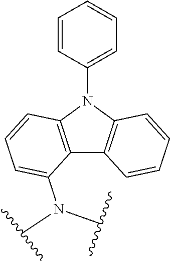 [SMILES: *N(*)c1cccc2c1c1ccccc1n2-c1ccccc1] | 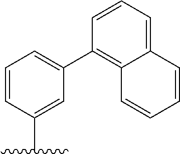 [SMILES: *c1cccc(-c2cccc3ccccc23)c1] |
| Q11 | 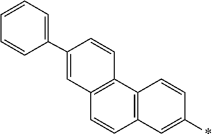 [SMILES: *c1ccc2c(ccc3cc(-c4ccccc4)ccc32)c1] | 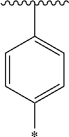 [SMILES: *c1ccc(*)cc1] | 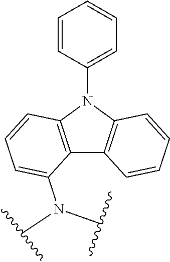 [SMILES: *N(*)c1cccc2c1c1ccccc1n2-c1ccccc1] | 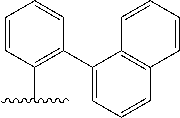 [SMILES: *c1ccccc1-c1cccc2ccccc12] |
| Q12 | 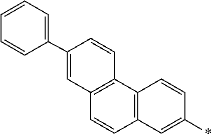 [SMILES: *c1ccc2c(ccc3cc(-c4ccccc4)ccc32)c1] | 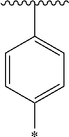 [SMILES: *c1ccc(*)cc1] | 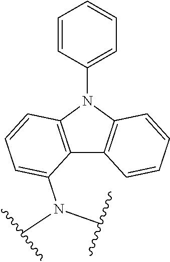 [SMILES: *N(*)c1cccc2c1c1ccccc1n2-c1ccccc1] | 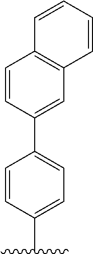 [SMILES: *c1ccc(-c2ccc3ccccc3c2)cc1] |
| Q13 | 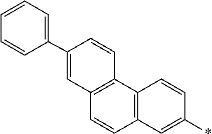 [SMILES: *c1ccc2c(ccc3cc(-c4ccccc4)ccc32)c1] | 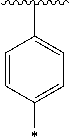 [SMILES: *c1ccc(*)cc1] | 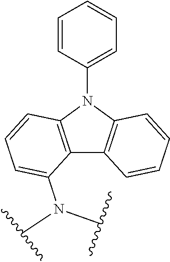 [SMILES: *N(*)c1cccc2c1c1ccccc1n2-c1ccccc1] | 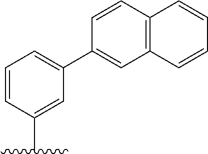 [SMILES: *c1cccc(-c2ccc3ccccc3c2)c1] |
| Q14 | 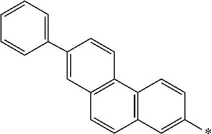 [SMILES: *c1ccc2c(ccc3cc(-c4ccccc4)ccc32)c1] | 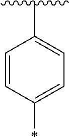 [SMILES: *c1ccc(*)cc1] | 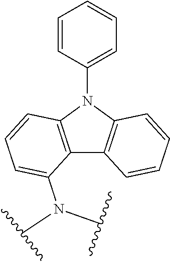 [SMILES: *N(*)c1cccc2c1c1ccccc1n2-c1ccccc1] | 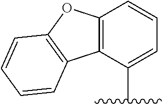 [SMILES: *c1cccc2oc3ccccc3c12] |
| Q15 | 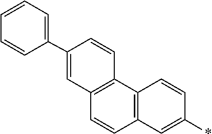 [SMILES: *c1ccc2c(ccc3cc(-c4ccccc4)ccc32)c1] | 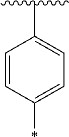 [SMILES: *c1ccc(*)cc1] | 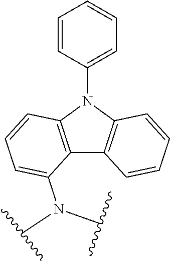 [SMILES: *N(*)c1cccc2c1c1ccccc1n2-c1ccccc1] | 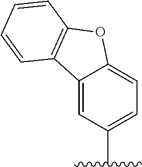 [SMILES: *c1ccc2oc3ccccc3c2c1] |
| Q16 | 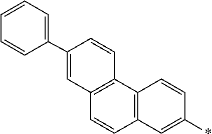 [SMILES: *c1ccc2c(ccc3cc(-c4ccccc4)ccc32)c1] | 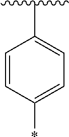 [SMILES: *c1ccc(*)cc1] | 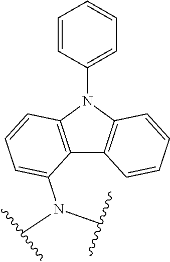 [SMILES: *N(*)c1cccc2c1c1ccccc1n2-c1ccccc1] | 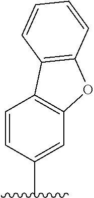 [SMILES: *c1ccc2c(c1)oc1ccccc12] |
| Q17 | 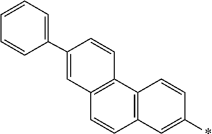 [SMILES: *c1ccc2c(ccc3cc(-c4ccccc4)ccc32)c1] | 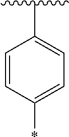 [SMILES: *c1ccc(*)cc1] | 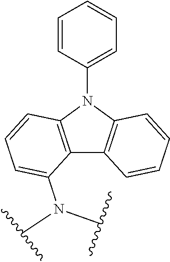 [SMILES: *N(*)c1cccc2c1c1ccccc1n2-c1ccccc1] | 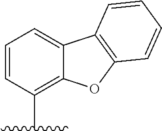 [SMILES: *c1cccc2c1oc1ccccc12] |
| Q18 | 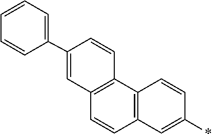 [SMILES: *c1ccc2c(ccc3cc(-c4ccccc4)ccc32)c1] | 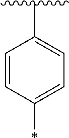 [SMILES: *c1ccc(*)cc1] | 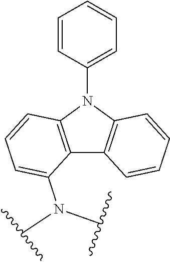 [SMILES: *N(*)c1cccc2c1c1ccccc1n2-c1ccccc1] | 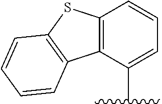 [SMILES: *c1cccc2sc3ccccc3c12] |
| Q19 | 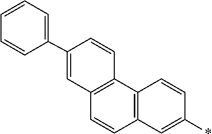 [SMILES: *c1ccc2c(ccc3cc(-c4ccccc4)ccc32)c1] | 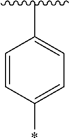 [SMILES: *c1ccc(*)cc1] | 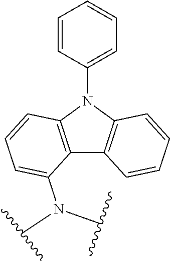 [SMILES: *N(*)c1cccc2c1c1ccccc1n2-c1ccccc1] | 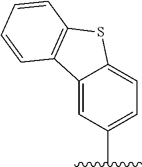 [SMILES: *c1ccc2sc3ccccc3c2c1] |
| Q20 | 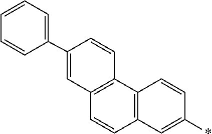 [SMILES: *c1ccc2c(ccc3cc(-c4ccccc4)ccc32)c1] | 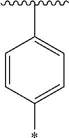 [SMILES: *c1ccc(*)cc1] | 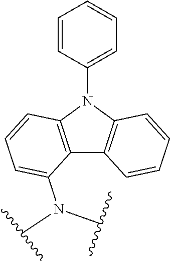 [SMILES: *N(*)c1cccc2c1c1ccccc1n2-c1ccccc1] | 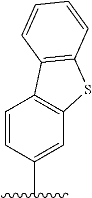 [SMILES: *c1ccc2c(c1)sc1ccccc12] |
| Q21 | 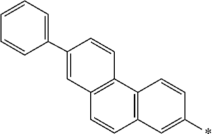 [SMILES: *c1ccc2c(ccc3cc(-c4ccccc4)ccc32)c1] | 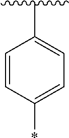 [SMILES: *c1ccc(*)cc1] | 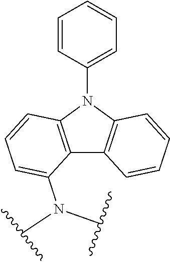 [SMILES: *N(*)c1cccc2c1c1ccccc1n2-c1ccccc1] | 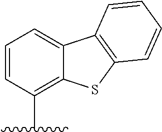 [SMILES: *c1cccc2c1sc1ccccc12] |
| Q22 | 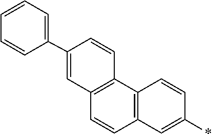 [SMILES: *c1ccc2c(ccc3cc(-c4ccccc4)ccc32)c1] | 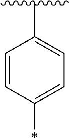 [SMILES: *c1ccc(*)cc1] | 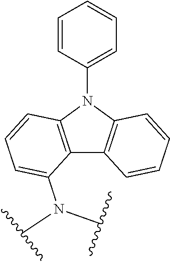 [SMILES: *N(*)c1cccc2c1c1ccccc1n2-c1ccccc1] | 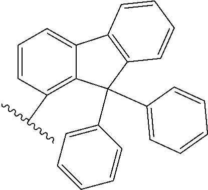 [SMILES: *c1cccc2c1C(c1ccccc1)(c1ccccc1)c1ccccc1-2] |
| Q23 | 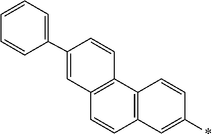 [SMILES: *c1ccc2c(ccc3cc(-c4ccccc4)ccc32)c1] | 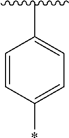 [SMILES: *c1ccc(*)cc1] | 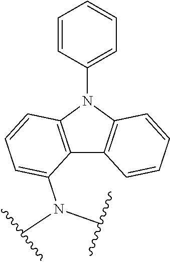 [SMILES: *N(*)c1cccc2c1c1ccccc1n2-c1ccccc1] | 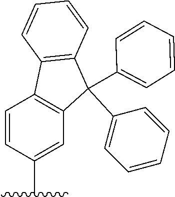 [SMILES: *c1ccc2c(c1)C(c1ccccc1)(c1ccccc1)c1ccccc1-2] |
| Q24 | 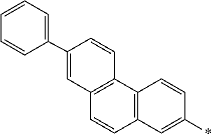 [SMILES: *c1ccc2c(ccc3cc(-c4ccccc4)ccc32)c1] | 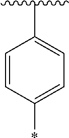 [SMILES: *c1ccc(*)cc1] | 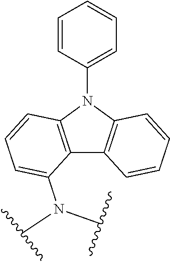 [SMILES: *N(*)c1cccc2c1c1ccccc1n2-c1ccccc1] | 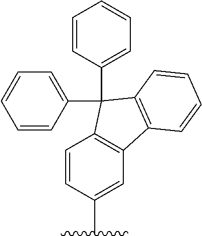 [SMILES: *c1ccc2c(c1)-c1ccccc1C2(c1ccccc1)c1ccccc1] |
| Q25 | 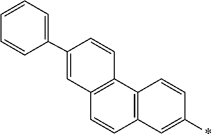 [SMILES: *c1ccc2c(ccc3cc(-c4ccccc4)ccc32)c1] | 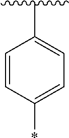 [SMILES: *c1ccc(*)cc1] | 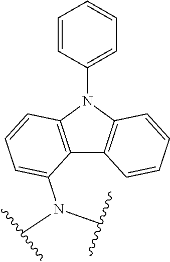 [SMILES: *N(*)c1cccc2c1c1ccccc1n2-c1ccccc1] | 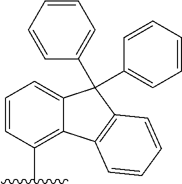 [SMILES: *c1cccc2c1-c1ccccc1C2(c1ccccc1)c1ccccc1] |
| Q26 | 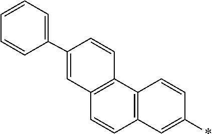 [SMILES: *c1ccc2c(ccc3cc(-c4ccccc4)ccc32)c1] | 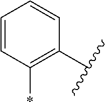 [SMILES: *c1ccccc1*] | 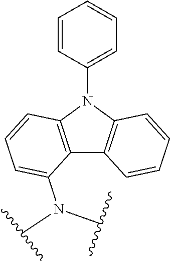 [SMILES: *N(*)c1cccc2c1c1ccccc1n2-c1ccccc1] | 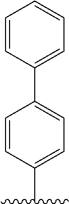 [SMILES: *c1ccc(-c2ccccc2)cc1] |
| Q27 | 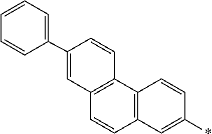 [SMILES: *c1ccc2c(ccc3cc(-c4ccccc4)ccc32)c1] | 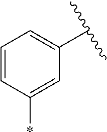 [SMILES: *c1cccc(*)c1] | 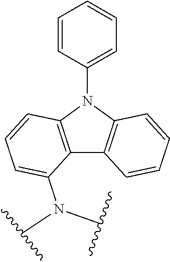 [SMILES: *N(*)c1cccc2c1c1ccccc1n2-c1ccccc1] | 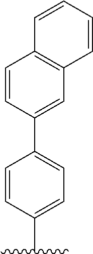 [SMILES: *c1ccc(-c2ccc3ccccc3c2)cc1] |
| Q28 | 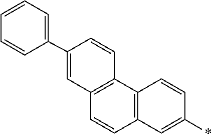 [SMILES: *c1ccc2c(ccc3cc(-c4ccccc4)ccc32)c1] | 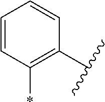 [SMILES: *c1ccccc1*] | 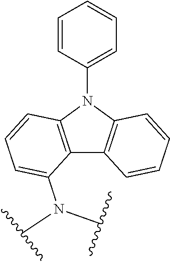 [SMILES: *N(*)c1cccc2c1c1ccccc1n2-c1ccccc1] | 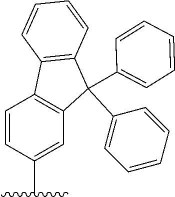 [SMILES: *c1ccc2c(c1)C(c1ccccc1)(c1ccccc1)c1ccccc1-2] |
| Q29 | 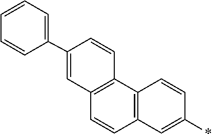 [SMILES: *c1ccc2c(ccc3cc(-c4ccccc4)ccc32)c1] | 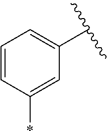 [SMILES: *c1cccc(*)c1] | 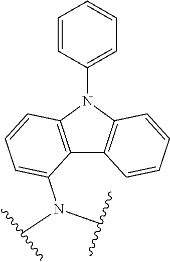 [SMILES: *N(*)c1cccc2c1c1ccccc1n2-c1ccccc1] | 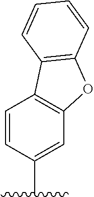 [SMILES: *c1ccc2c(c1)oc1ccccc12] |
| Q30 | 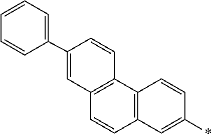 [SMILES: *c1ccc2c(ccc3cc(-c4ccccc4)ccc32)c1] | 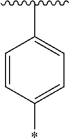 [SMILES: *c1ccc(*)cc1] | 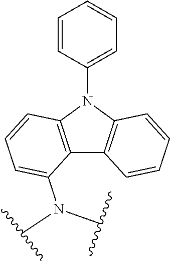 [SMILES: *N(*)c1cccc2c1c1ccccc1n2-c1ccccc1] | 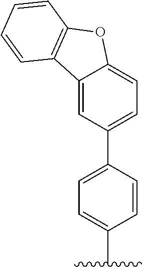 [SMILES: *c1ccc(-c2ccc3oc4ccccc4c3c2)cc1] |
| Q31 | 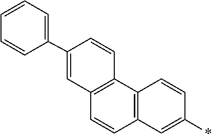 [SMILES: *c1ccc2c(ccc3cc(-c4ccccc4)ccc32)c1] | 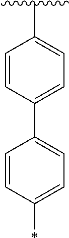 [SMILES: *c1ccc(-c2ccc(*)cc2)cc1] | 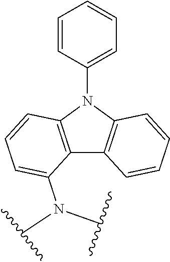 [SMILES: *N(*)c1cccc2c1c1ccccc1n2-c1ccccc1] | 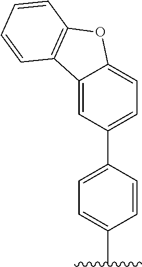 [SMILES: *c1ccc(-c2ccc3oc4ccccc4c3c2)cc1] |
| Q32 | 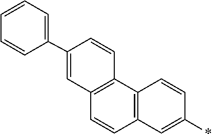 [SMILES: *c1ccc2c(ccc3cc(-c4ccccc4)ccc32)c1] | 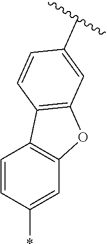 [SMILES: *c1ccc2c(c1)oc1cc(*)ccc12] | 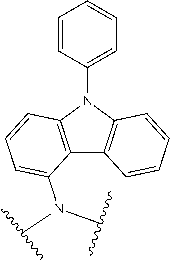 [SMILES: *N(*)c1cccc2c1c1ccccc1n2-c1ccccc1] | 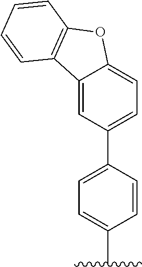 [SMILES: *c1ccc(-c2ccc3oc4ccccc4c3c2)cc1] |
| Q33 | 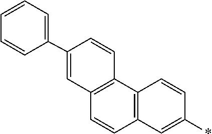 [SMILES: *c1ccc2c(ccc3cc(-c4ccccc4)ccc32)c1] | 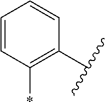 [SMILES: *c1ccccc1*] | 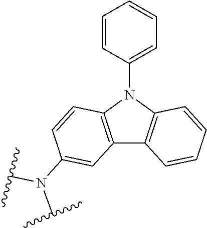 [SMILES: *N(*)c1ccc2c(c1)c1ccccc1n2-c1ccccc1] | 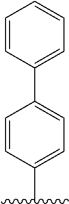 [SMILES: *c1ccc(-c2ccccc2)cc1] |
| Q34 | 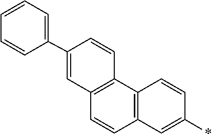 [SMILES: *c1ccc2c(ccc3cc(-c4ccccc4)ccc32)c1] | 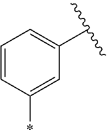 [SMILES: *c1cccc(*)c1] | 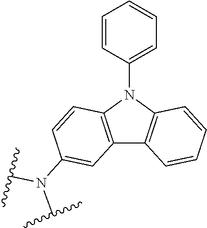 [SMILES: *N(*)c1ccc2c(c1)c1ccccc1n2-c1ccccc1] | 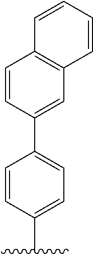 [SMILES: *c1ccc(-c2ccc3ccccc3c2)cc1] |
| Q35 | 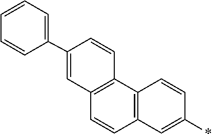 [SMILES: *c1ccc2c(ccc3cc(-c4ccccc4)ccc32)c1] | 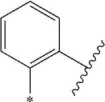 [SMILES: *c1ccccc1*] | 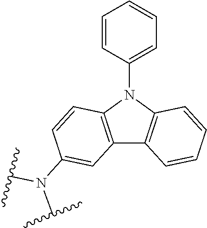 [SMILES: *N(*)c1ccc2c(c1)c1ccccc1n2-c1ccccc1] | 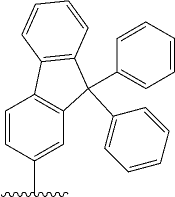 [SMILES: *c1ccc2c(c1)C(c1ccccc1)(c1ccccc1)c1ccccc1-2] |
| Q36 | 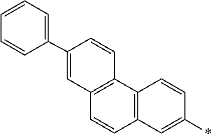 [SMILES: *c1ccc2c(ccc3cc(-c4ccccc4)ccc32)c1] | 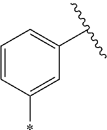 [SMILES: *c1cccc(*)c1] | 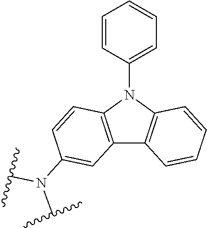 [SMILES: *N(*)c1ccc2c(c1)c1ccccc1n2-c1ccccc1] | 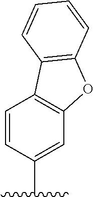 [SMILES: *c1ccc2c(c1)oc1ccccc12] |
| Q37 | 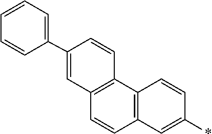 [SMILES: *c1ccc2c(ccc3cc(-c4ccccc4)ccc32)c1] | 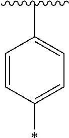 [SMILES: *c1ccc(*)cc1] | 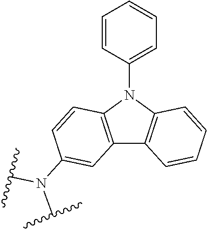 [SMILES: *N(*)c1ccc2c(c1)c1ccccc1n2-c1ccccc1] | 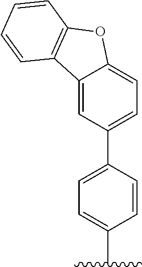 [SMILES: *c1ccc(-c2ccc3oc4ccccc4c3c2)cc1] |
| Q38 | 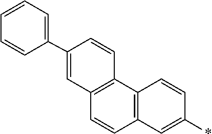 [SMILES: *c1ccc2c(ccc3cc(-c4ccccc4)ccc32)c1] | 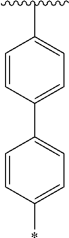 [SMILES: *c1ccc(-c2ccc(*)cc2)cc1] | 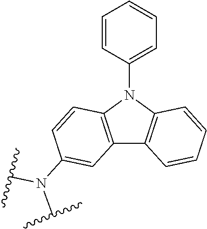 [SMILES: *N(*)c1ccc2c(c1)c1ccccc1n2-c1ccccc1] | 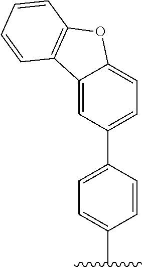 [SMILES: *c1ccc(-c2ccc3oc4ccccc4c3c2)cc1] |
| Q39 | 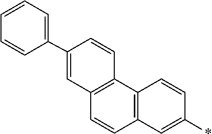 [SMILES: *c1ccc2c(ccc3cc(-c4ccccc4)ccc32)c1] | 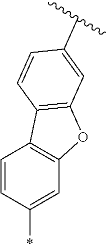 [SMILES: *c1ccc2c(c1)oc1cc(*)ccc12] | 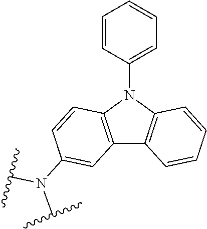 [SMILES: *N(*)c1ccc2c(c1)c1ccccc1n2-c1ccccc1] | 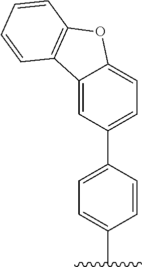 [SMILES: *c1ccc(-c2ccc3oc4ccccc4c3c2)cc1] |
| Q40 | 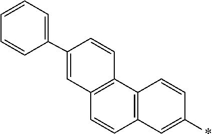 [SMILES: *c1ccc2c(ccc3cc(-c4ccccc4)ccc32)c1] | 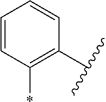 [SMILES: *c1ccccc1*] | 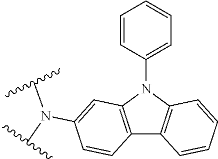 [SMILES: *N(*)c1ccc2c3ccccc3n(-c3ccccc3)c2c1] | 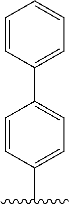 [SMILES: *c1ccc(-c2ccccc2)cc1] |
| Q41 | 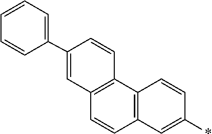 [SMILES: *c1ccc2c(ccc3cc(-c4ccccc4)ccc32)c1] | 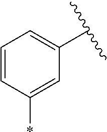 [SMILES: *c1cccc(*)c1] | 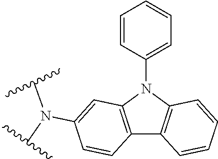 [SMILES: *N(*)c1ccc2c3ccccc3n(-c3ccccc3)c2c1] | 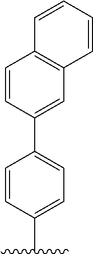 [SMILES: *c1ccc(-c2ccc3ccccc3c2)cc1] |
| Q42 | 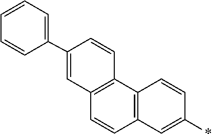 [SMILES: *c1ccc2c(ccc3cc(-c4ccccc4)ccc32)c1] | 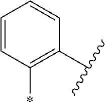 [SMILES: *c1ccccc1*] | 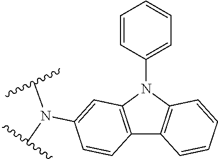 [SMILES: *N(*)c1ccc2c3ccccc3n(-c3ccccc3)c2c1] | 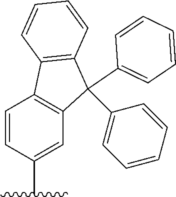 [SMILES: *c1ccc2c(c1)C(c1ccccc1)(c1ccccc1)c1ccccc1-2] |
| Q43 | 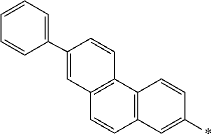 [SMILES: *c1ccc2c(ccc3cc(-c4ccccc4)ccc32)c1] | 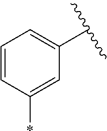 [SMILES: *c1cccc(*)c1] | 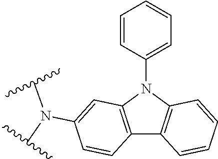 [SMILES: *N(*)c1ccc2c3ccccc3n(-c3ccccc3)c2c1] | 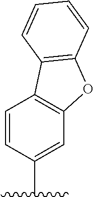 [SMILES: *c1ccc2c(c1)oc1ccccc12] |
| Q44 | 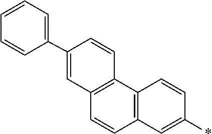 [SMILES: *c1ccc2c(ccc3cc(-c4ccccc4)ccc32)c1] | 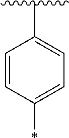 [SMILES: *c1ccc(*)cc1] | 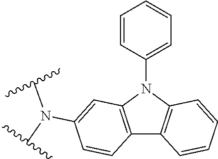 [SMILES: *N(*)c1ccc2c3ccccc3n(-c3ccccc3)c2c1] | 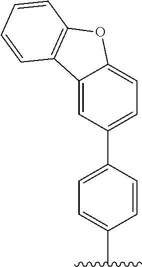 [SMILES: *c1ccc(-c2ccc3oc4ccccc4c3c2)cc1] |
| Q45 | 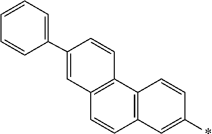 [SMILES: *c1ccc2c(ccc3cc(-c4ccccc4)ccc32)c1] | 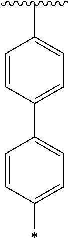 [SMILES: *c1ccc(-c2ccc(*)cc2)cc1] | 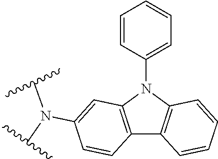 [SMILES: *N(*)c1ccc2c3ccccc3n(-c3ccccc3)c2c1] | 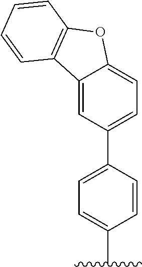 [SMILES: *c1ccc(-c2ccc3oc4ccccc4c3c2)cc1] |
| Q46 | 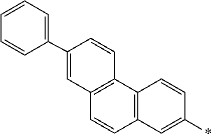 [SMILES: *c1ccc2c(ccc3cc(-c4ccccc4)ccc32)c1] | 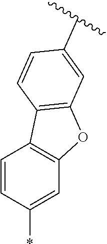 [SMILES: *c1ccc2c(c1)oc1cc(*)ccc12] | 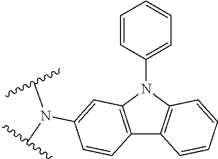 [SMILES: *N(*)c1ccc2c3ccccc3n(-c3ccccc3)c2c1] | 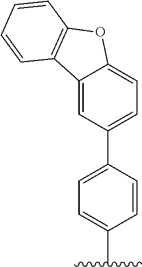 [SMILES: *c1ccc(-c2ccc3oc4ccccc4c3c2)cc1] |
| S1 | 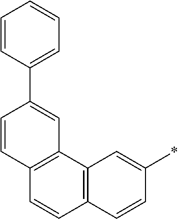 [SMILES: *c1ccc2ccc3ccc(-c4ccccc4)cc3c2c1] | 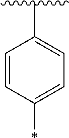 [SMILES: *c1ccc(*)cc1] | 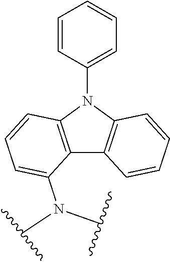 [SMILES: *N(*)c1cccc2c1c1ccccc1n2-c1ccccc1] | 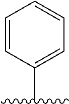 [SMILES: *c1ccccc1] |
| S2 | 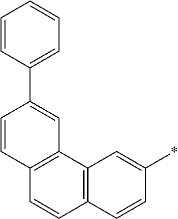 [SMILES: *c1ccc2ccc3ccc(-c4ccccc4)cc3c2c1] | 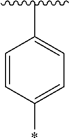 [SMILES: *c1ccc(*)cc1] | 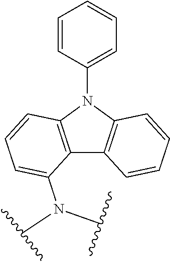 [SMILES: *N(*)c1cccc2c1c1ccccc1n2-c1ccccc1] | 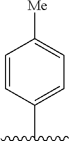 [SMILES: *c1ccc(C)cc1] |
| S3 | 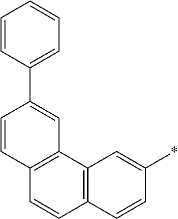 [SMILES: *c1ccc2ccc3ccc(-c4ccccc4)cc3c2c1] | 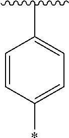 [SMILES: *c1ccc(*)cc1] | 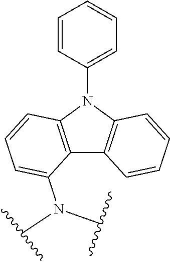 [SMILES: *N(*)c1cccc2c1c1ccccc1n2-c1ccccc1] | 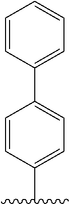 [SMILES: *c1ccc(-c2ccccc2)cc1] |
| S4 | 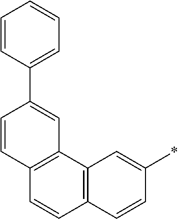 [SMILES: *c1ccc2ccc3ccc(-c4ccccc4)cc3c2c1] | 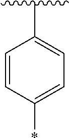 [SMILES: *c1ccc(*)cc1] | 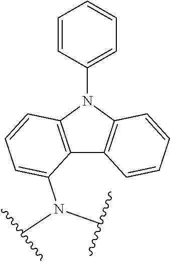 [SMILES: *N(*)c1cccc2c1c1ccccc1n2-c1ccccc1] | 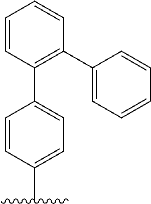 [SMILES: *c1ccc(-c2ccccc2-c2ccccc2)cc1] |
| S5 | 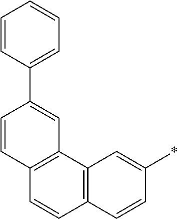 [SMILES: *c1ccc2ccc3ccc(-c4ccccc4)cc3c2c1] | 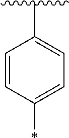 [SMILES: *c1ccc(*)cc1] | 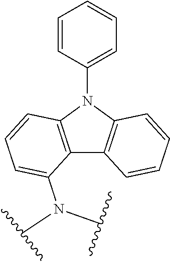 [SMILES: *N(*)c1cccc2c1c1ccccc1n2-c1ccccc1] | 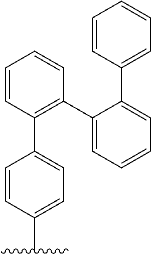 [SMILES: *c1ccc(-c2ccccc2-c2ccccc2-c2ccccc2)cc1] |
| S6 | 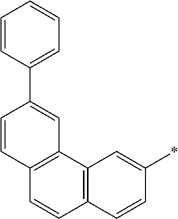 [SMILES: *c1ccc2ccc3ccc(-c4ccccc4)cc3c2c1] | 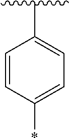 [SMILES: *c1ccc(*)cc1] | 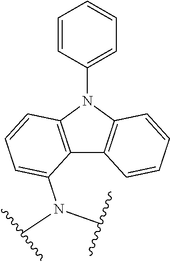 [SMILES: *N(*)c1cccc2c1c1ccccc1n2-c1ccccc1] | 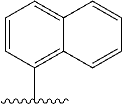 [SMILES: *c1cccc2ccccc12] |
| S7 | 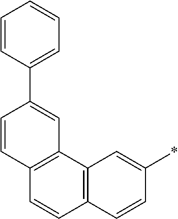 [SMILES: *c1ccc2ccc3ccc(-c4ccccc4)cc3c2c1] | 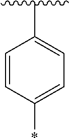 [SMILES: *c1ccc(*)cc1] | 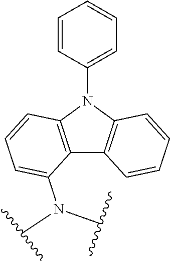 [SMILES: *N(*)c1cccc2c1c1ccccc1n2-c1ccccc1] | 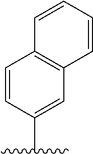 [SMILES: *c1ccc2ccccc2c1] |
| S8 | 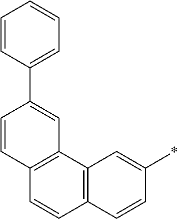 [SMILES: *c1ccc2ccc3ccc(-c4ccccc4)cc3c2c1] | 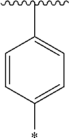 [SMILES: *c1ccc(*)cc1] | 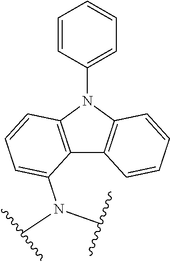 [SMILES: *N(*)c1cccc2c1c1ccccc1n2-c1ccccc1] | 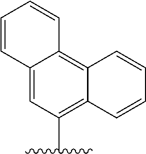 [SMILES: *c1cc2ccccc2c2ccccc12] |
| S9 | 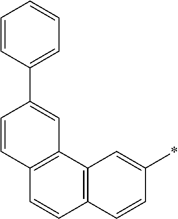 [SMILES: *c1ccc2ccc3ccc(-c4ccccc4)cc3c2c1] | 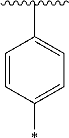 [SMILES: *c1ccc(*)cc1] | 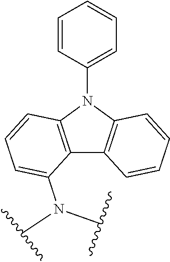 [SMILES: *N(*)c1cccc2c1c1ccccc1n2-c1ccccc1] | 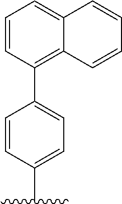 [SMILES: *c1ccc(-c2cccc3ccccc23)cc1] |
| S10 | 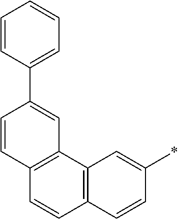 [SMILES: *c1ccc2ccc3ccc(-c4ccccc4)cc3c2c1] | 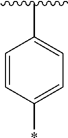 [SMILES: *c1ccc(*)cc1] | 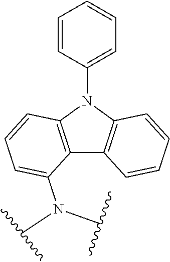 [SMILES: *N(*)c1cccc2c1c1ccccc1n2-c1ccccc1] | 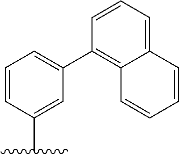 [SMILES: *c1cccc(-c2cccc3ccccc23)c1] |
| S11 | 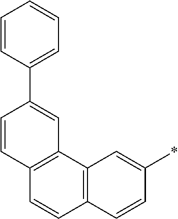 [SMILES: *c1ccc2ccc3ccc(-c4ccccc4)cc3c2c1] | 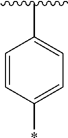 [SMILES: *c1ccc(*)cc1] | 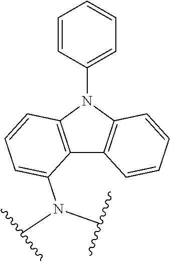 [SMILES: *N(*)c1cccc2c1c1ccccc1n2-c1ccccc1] | 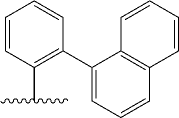 [SMILES: *c1ccccc1-c1cccc2ccccc12] |
| S12 | 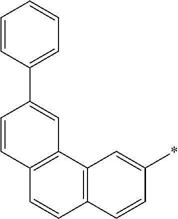 [SMILES: *c1ccc2ccc3ccc(-c4ccccc4)cc3c2c1] | 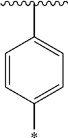 [SMILES: *c1ccc(*)cc1] | 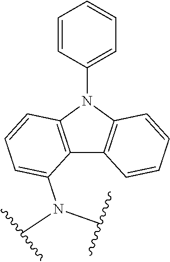 [SMILES: *N(*)c1cccc2c1c1ccccc1n2-c1ccccc1] | 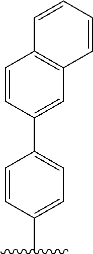 [SMILES: *c1ccc(-c2ccc3ccccc3c2)cc1] |
| S13 | 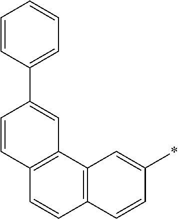 [SMILES: *c1ccc2ccc3ccc(-c4ccccc4)cc3c2c1] | 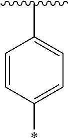 [SMILES: *c1ccc(*)cc1] | 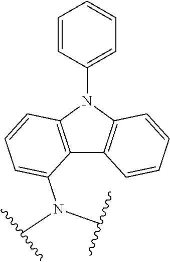 [SMILES: *N(*)c1cccc2c1c1ccccc1n2-c1ccccc1] | 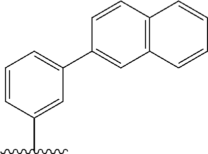 [SMILES: *c1cccc(-c2ccc3ccccc3c2)c1] |
| S14 | 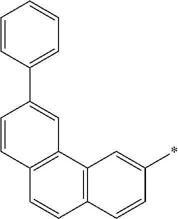 [SMILES: *c1ccc2ccc3ccc(-c4ccccc4)cc3c2c1] | 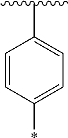 [SMILES: *c1ccc(*)cc1] | 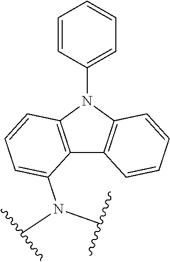 [SMILES: *N(*)c1cccc2c1c1ccccc1n2-c1ccccc1] | 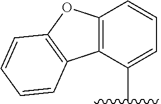 [SMILES: *c1cccc2oc3ccccc3c12] |
| S15 | 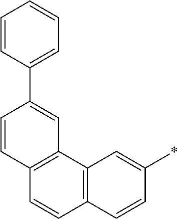 [SMILES: *c1ccc2ccc3ccc(-c4ccccc4)cc3c2c1] | 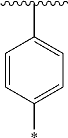 [SMILES: *c1ccc(*)cc1] | 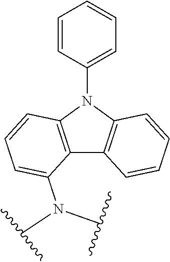 [SMILES: *N(*)c1cccc2c1c1ccccc1n2-c1ccccc1] | 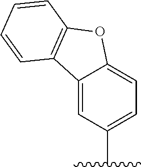 [SMILES: *c1ccc2oc3ccccc3c2c1] |
| S16 | 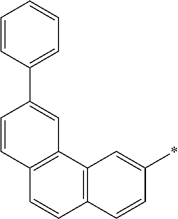 [SMILES: *c1ccc2ccc3ccc(-c4ccccc4)cc3c2c1] | 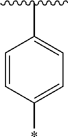 [SMILES: *c1ccc(*)cc1] | 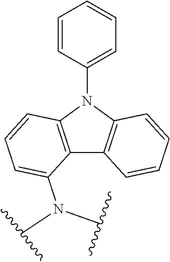 [SMILES: *N(*)c1cccc2c1c1ccccc1n2-c1ccccc1] | 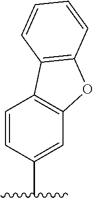 [SMILES: *c1ccc2c(c1)oc1ccccc12] |
| S17 | 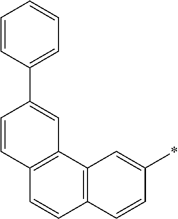 [SMILES: *c1ccc2ccc3ccc(-c4ccccc4)cc3c2c1] | 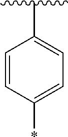 [SMILES: *c1ccc(*)cc1] | 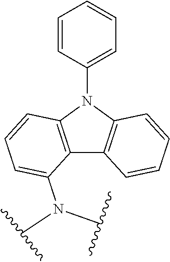 [SMILES: *N(*)c1cccc2c1c1ccccc1n2-c1ccccc1] | 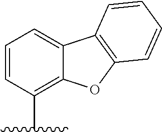 [SMILES: *c1cccc2c1oc1ccccc12] |
| S18 | 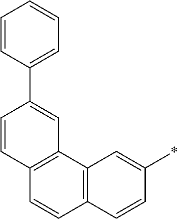 [SMILES: *c1ccc2ccc3ccc(-c4ccccc4)cc3c2c1] | 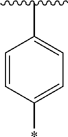 [SMILES: *c1ccc(*)cc1] | 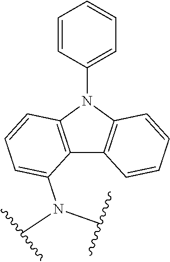 [SMILES: *N(*)c1cccc2c1c1ccccc1n2-c1ccccc1] | 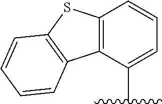 [SMILES: *c1cccc2sc3ccccc3c12] |
| S19 | 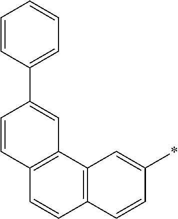 [SMILES: *c1ccc2ccc3ccc(-c4ccccc4)cc3c2c1] | 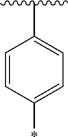 [SMILES: *c1ccc(*)cc1] | 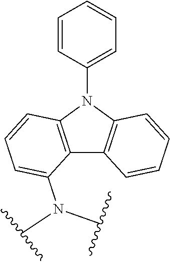 [SMILES: *N(*)c1cccc2c1c1ccccc1n2-c1ccccc1] | 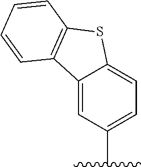 [SMILES: *c1ccc2sc3ccccc3c2c1] |
| S20 | 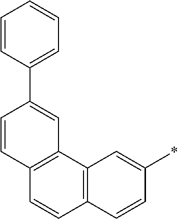 [SMILES: *c1ccc2ccc3ccc(-c4ccccc4)cc3c2c1] | 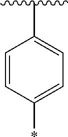 [SMILES: *c1ccc(*)cc1] | 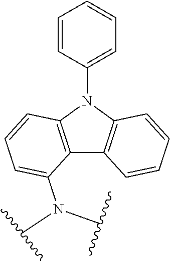 [SMILES: *N(*)c1cccc2c1c1ccccc1n2-c1ccccc1] | 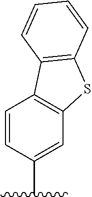 [SMILES: *c1ccc2c(c1)sc1ccccc12] |
| S21 | 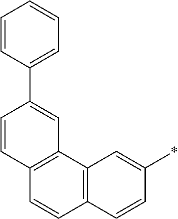 [SMILES: *c1ccc2ccc3ccc(-c4ccccc4)cc3c2c1] | 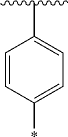 [SMILES: *c1ccc(*)cc1] | 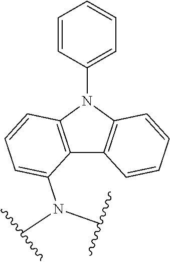 [SMILES: *N(*)c1cccc2c1c1ccccc1n2-c1ccccc1] | 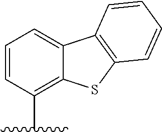 [SMILES: *c1cccc2c1sc1ccccc12] |
| S22 | 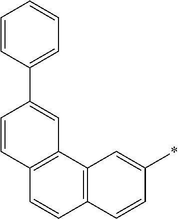 [SMILES: *c1ccc2ccc3ccc(-c4ccccc4)cc3c2c1] | 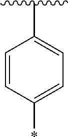 [SMILES: *c1ccc(*)cc1] | 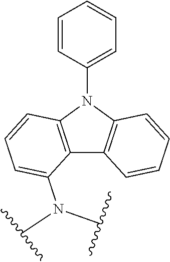 [SMILES: *N(*)c1cccc2c1c1ccccc1n2-c1ccccc1] | 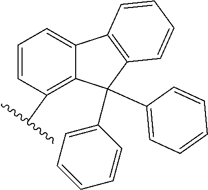 [SMILES: *c1cccc2c1C(c1ccccc1)(c1ccccc1)c1ccccc1-2] |
| S23 | 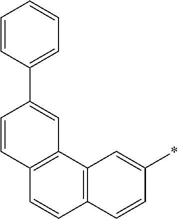 [SMILES: *c1ccc2ccc3ccc(-c4ccccc4)cc3c2c1] | 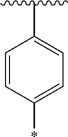 [SMILES: *c1ccc(*)cc1] | 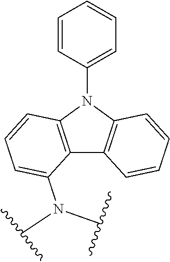 [SMILES: *N(*)c1cccc2c1c1ccccc1n2-c1ccccc1] | 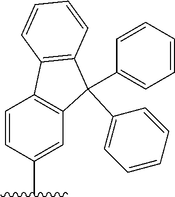 [SMILES: *c1ccc2c(c1)C(c1ccccc1)(c1ccccc1)c1ccccc1-2] |
| S24 | 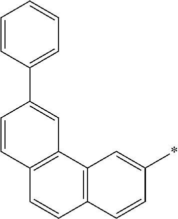 [SMILES: *c1ccc2ccc3ccc(-c4ccccc4)cc3c2c1] | 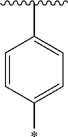 [SMILES: *c1ccc(*)cc1] | 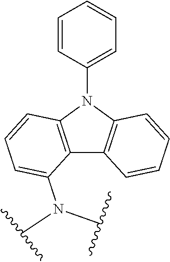 [SMILES: *N(*)c1cccc2c1c1ccccc1n2-c1ccccc1] | 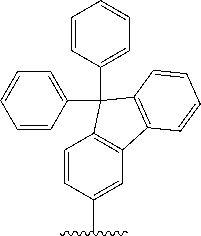 [SMILES: *c1ccc2c(c1)-c1ccccc1C2(c1ccccc1)c1ccccc1] |
| S25 | 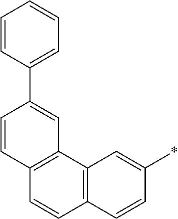 [SMILES: *c1ccc2ccc3ccc(-c4ccccc4)cc3c2c1] | 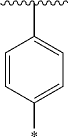 [SMILES: *c1ccc(*)cc1] | 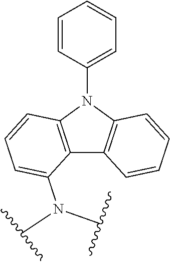 [SMILES: *N(*)c1cccc2c1c1ccccc1n2-c1ccccc1] | 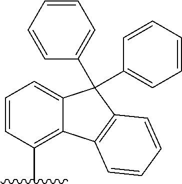 [SMILES: *c1cccc2c1-c1ccccc1C2(c1ccccc1)c1ccccc1] |
| S26 | 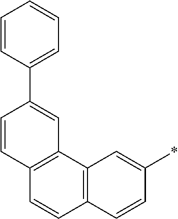 [SMILES: *c1ccc2ccc3ccc(-c4ccccc4)cc3c2c1] | 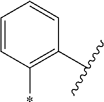 [SMILES: *c1ccccc1*] | 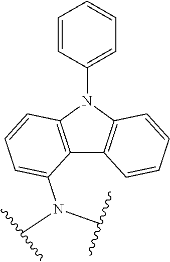 [SMILES: *N(*)c1cccc2c1c1ccccc1n2-c1ccccc1] | 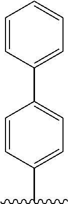 [SMILES: *c1ccc(-c2ccccc2)cc1] |
| S27 | 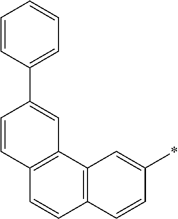 [SMILES: *c1ccc2ccc3ccc(-c4ccccc4)cc3c2c1] | 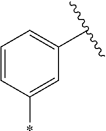 [SMILES: *c1cccc(*)c1] | 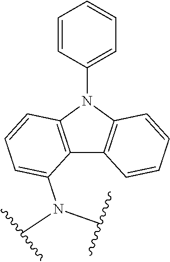 [SMILES: *N(*)c1cccc2c1c1ccccc1n2-c1ccccc1] | 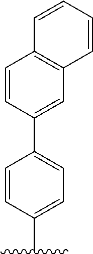 [SMILES: *c1ccc(-c2ccc3ccccc3c2)cc1] |
| S28 | 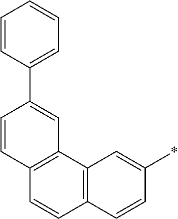 [SMILES: *c1ccc2ccc3ccc(-c4ccccc4)cc3c2c1] | 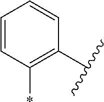 [SMILES: *c1ccccc1*] | 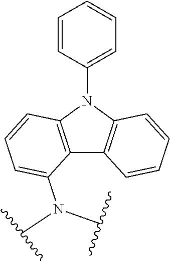 [SMILES: *N(*)c1cccc2c1c1ccccc1n2-c1ccccc1] | 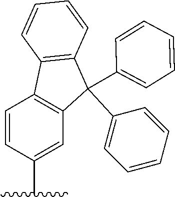 [SMILES: *c1ccc2c(c1)C(c1ccccc1)(c1ccccc1)c1ccccc1-2] |
| S29 | 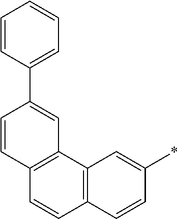 [SMILES: *c1ccc2ccc3ccc(-c4ccccc4)cc3c2c1] | 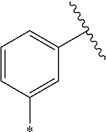 [SMILES: *c1cccc(*)c1] | 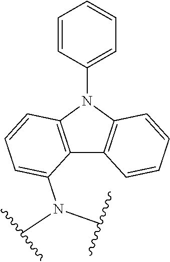 [SMILES: *N(*)c1cccc2c1c1ccccc1n2-c1ccccc1] | 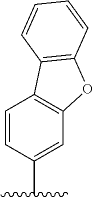 [SMILES: *c1ccc2c(c1)oc1ccccc12] |
| S30 | 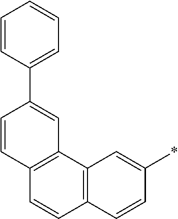 [SMILES: *c1ccc2ccc3ccc(-c4ccccc4)cc3c2c1] | 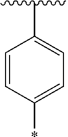 [SMILES: *c1ccc(*)cc1] | 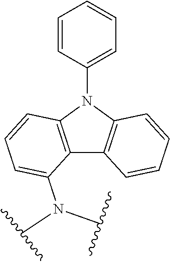 [SMILES: *N(*)c1cccc2c1c1ccccc1n2-c1ccccc1] | 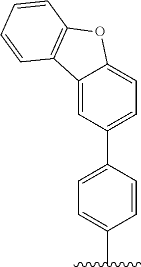 [SMILES: *c1ccc(-c2ccc3oc4ccccc4c3c2)cc1] |
| S31 | 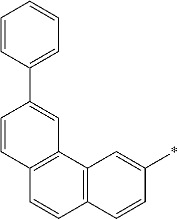 [SMILES: *c1ccc2ccc3ccc(-c4ccccc4)cc3c2c1] | 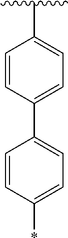 [SMILES: *c1ccc(-c2ccc(*)cc2)cc1] | 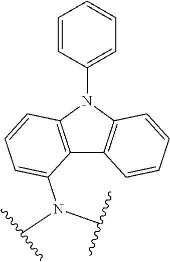 [SMILES: *N(*)c1cccc2c1c1ccccc1n2-c1ccccc1] | 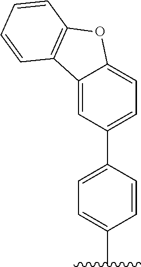 [SMILES: *c1ccc(-c2ccc3oc4ccccc4c3c2)cc1] |
| S32 | 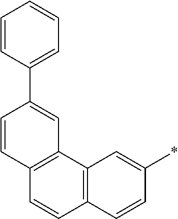 [SMILES: *c1ccc2ccc3ccc(-c4ccccc4)cc3c2c1] | 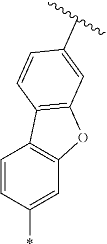 [SMILES: *c1ccc2c(c1)oc1cc(*)ccc12] | 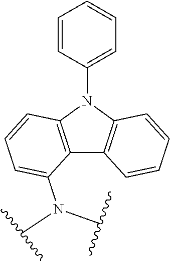 [SMILES: *N(*)c1cccc2c1c1ccccc1n2-c1ccccc1] | 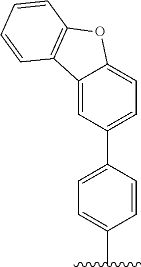 [SMILES: *c1ccc(-c2ccc3oc4ccccc4c3c2)cc1] |
| S33 | 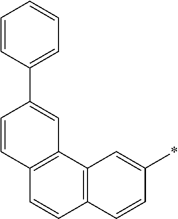 [SMILES: *c1ccc2ccc3ccc(-c4ccccc4)cc3c2c1] | 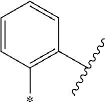 [SMILES: *c1ccccc1*] | 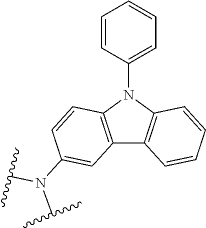 [SMILES: *N(*)c1ccc2c(c1)c1ccccc1n2-c1ccccc1] | 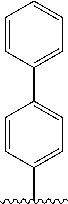 [SMILES: *c1ccc(-c2ccccc2)cc1] |
| S34 | 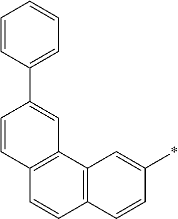 [SMILES: *c1ccc2ccc3ccc(-c4ccccc4)cc3c2c1] | 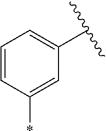 [SMILES: *c1cccc(*)c1] | 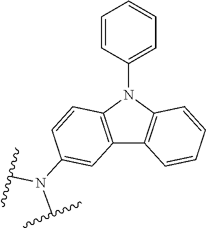 [SMILES: *N(*)c1ccc2c(c1)c1ccccc1n2-c1ccccc1] | 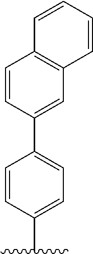 [SMILES: *c1ccc(-c2ccc3ccccc3c2)cc1] |
| S35 | 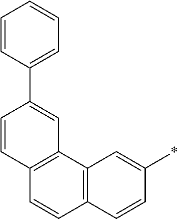 [SMILES: *c1ccc2ccc3ccc(-c4ccccc4)cc3c2c1] | 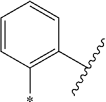 [SMILES: *c1ccccc1*] | 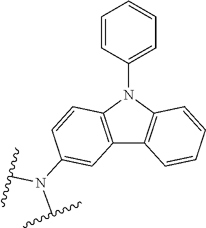 [SMILES: *N(*)c1ccc2c(c1)c1ccccc1n2-c1ccccc1] | 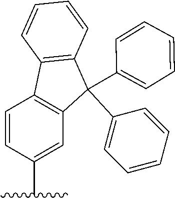 [SMILES: *c1ccc2c(c1)C(c1ccccc1)(c1ccccc1)c1ccccc1-2] |
| S36 | 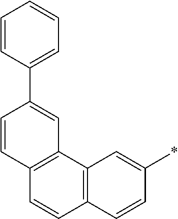 [SMILES: *c1ccc2ccc3ccc(-c4ccccc4)cc3c2c1] | 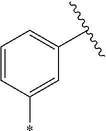 [SMILES: *c1cccc(*)c1] | 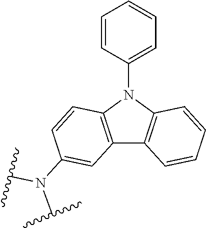 [SMILES: *N(*)c1ccc2c(c1)c1ccccc1n2-c1ccccc1] | 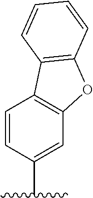 [SMILES: *c1ccc2c(c1)oc1ccccc12] |
| S37 | 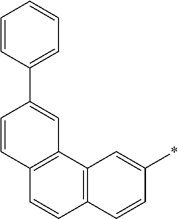 [SMILES: *c1ccc2ccc3ccc(-c4ccccc4)cc3c2c1] | 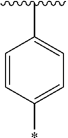 [SMILES: *c1ccc(*)cc1] | 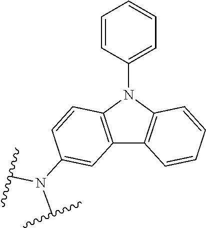 [SMILES: *N(*)c1ccc2c(c1)c1ccccc1n2-c1ccccc1] | 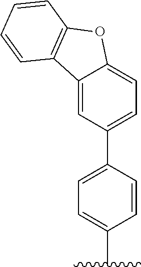 [SMILES: *c1ccc(-c2ccc3oc4ccccc4c3c2)cc1] |
| S38 | 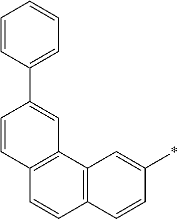 [SMILES: *c1ccc2ccc3ccc(-c4ccccc4)cc3c2c1] | 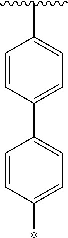 [SMILES: *c1ccc(-c2ccc(*)cc2)cc1] | 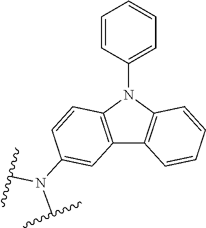 [SMILES: *N(*)c1ccc2c(c1)c1ccccc1n2-c1ccccc1] | 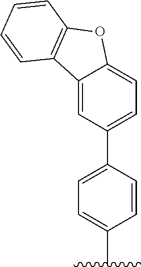 [SMILES: *c1ccc(-c2ccc3oc4ccccc4c3c2)cc1] |
| S39 | 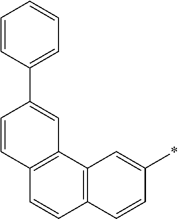 [SMILES: *c1ccc2ccc3ccc(-c4ccccc4)cc3c2c1] | 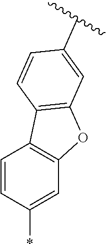 [SMILES: *c1ccc2c(c1)oc1cc(*)ccc12] | 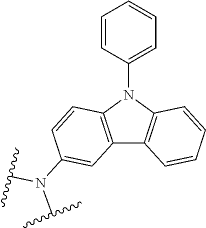 [SMILES: *N(*)c1ccc2c(c1)c1ccccc1n2-c1ccccc1] | 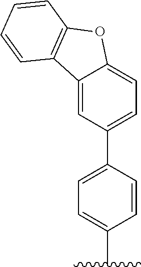 [SMILES: *c1ccc(-c2ccc3oc4ccccc4c3c2)cc1] |
| S40 | 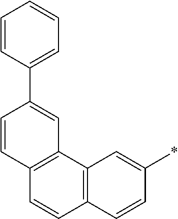 [SMILES: *c1ccc2ccc3ccc(-c4ccccc4)cc3c2c1] | 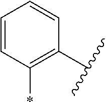 [SMILES: *c1ccccc1*] | 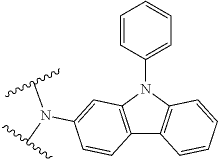 [SMILES: *N(*)c1ccc2c3ccccc3n(-c3ccccc3)c2c1] | 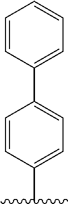 [SMILES: *c1ccc(-c2ccccc2)cc1] |
| S41 | 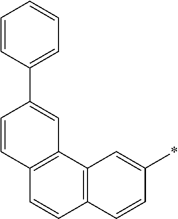 [SMILES: *c1ccc2ccc3ccc(-c4ccccc4)cc3c2c1] | 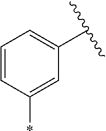 [SMILES: *c1cccc(*)c1] | 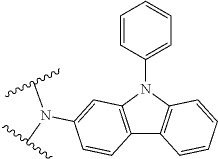 [SMILES: *N(*)c1ccc2c3ccccc3n(-c3ccccc3)c2c1] | 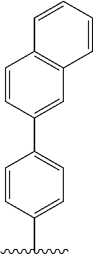 [SMILES: *c1ccc(-c2ccc3ccccc3c2)cc1] |
| S42 | 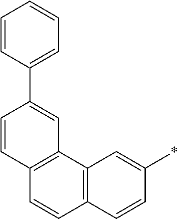 [SMILES: *c1ccc2ccc3ccc(-c4ccccc4)cc3c2c1] | 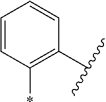 [SMILES: *c1ccccc1*] | 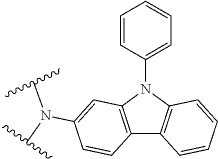 [SMILES: *N(*)c1ccc2c3ccccc3n(-c3ccccc3)c2c1] | 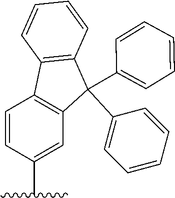 [SMILES: *c1ccc2c(c1)C(c1ccccc1)(c1ccccc1)c1ccccc1-2] |
| S43 | 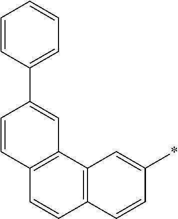 [SMILES: *c1ccc2ccc3ccc(-c4ccccc4)cc3c2c1] | 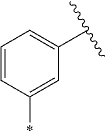 [SMILES: *c1cccc(*)c1] | 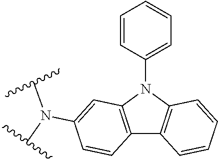 [SMILES: *N(*)c1ccc2c3ccccc3n(-c3ccccc3)c2c1] | 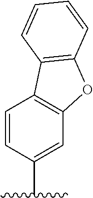 [SMILES: *c1ccc2c(c1)oc1ccccc12] |
| S44 | 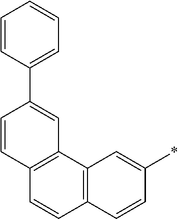 [SMILES: *c1ccc2ccc3ccc(-c4ccccc4)cc3c2c1] | 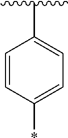 [SMILES: *c1ccc(*)cc1] | 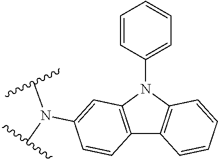 [SMILES: *N(*)c1ccc2c3ccccc3n(-c3ccccc3)c2c1] | 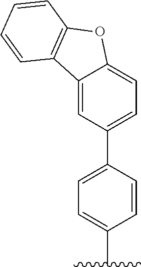 [SMILES: *c1ccc(-c2ccc3oc4ccccc4c3c2)cc1] |
| S45 | 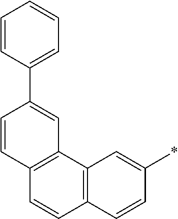 [SMILES: *c1ccc2ccc3ccc(-c4ccccc4)cc3c2c1] | 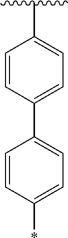 [SMILES: *c1ccc(-c2ccc(*)cc2)cc1] | 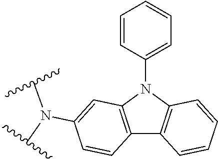 [SMILES: *N(*)c1ccc2c3ccccc3n(-c3ccccc3)c2c1] | 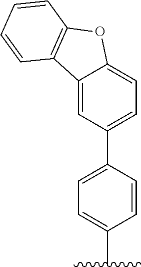 [SMILES: *c1ccc(-c2ccc3oc4ccccc4c3c2)cc1] |
| S46 | 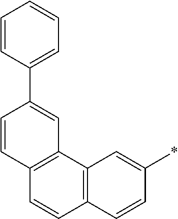 [SMILES: *c1ccc2ccc3ccc(-c4ccccc4)cc3c2c1] | 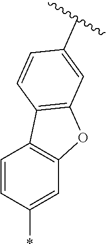 [SMILES: *c1ccc2c(c1)oc1cc(*)ccc12] | 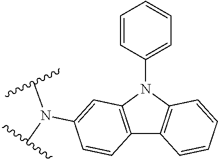 [SMILES: *N(*)c1ccc2c3ccccc3n(-c3ccccc3)c2c1] | 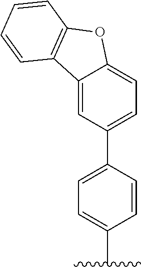 [SMILES: *c1ccc(-c2ccc3oc4ccccc4c3c2)cc1] |

The amine compound of the present disclosure includes a carbazole group directly linked to the central nitrogen atom, and a naphthyl group that is linked to the central nitrogen atom and is substituted with an aryl group, and thus the hole transport ability of the molecule may be improved. In some embodiments, the amine compound further includes, as a substituent, a fluorene group that causes a steric effect, and thus the stability of the molecule may be increased.

The hole transport region HTR included in the light emitting element ED of the present disclosure includes the above-described amine compound, and thus the hole transport ability may be improved and the driving voltage of the element may be reduced.

The hole transport region HTR in the light emitting element ED of an embodiment may further include a compound represented by Formula H-1:

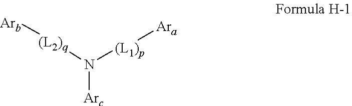

In Formula H-1, Arₐ and Ar_{b} may be each independently a substituted or unsubstituted aryl group having 6 to 30 ring-forming carbon atoms, or a substituted or unsubstituted heteroaryl group having 2 to 30 ring-forming carbon atoms.

Ar_{c} may be a substituted or unsubstituted aryl group having 6 to 30 ring-forming carbon atoms.

L₁ and L₂ may be each independently a direct linkage, a substituted or unsubstituted arylene group having 6 to 30 ring-forming carbon atoms, or a substituted or unsubstituted heteroarylene group having 2 to 30 ring-forming carbon atoms.

p and q may be each independently an integer from 0 to 10. In some embodiments, when p or q is an integer of 2 or greater, a plurality of L₁s and L₂s may be each independently a substituted or unsubstituted arylene group having 6 to 30 ring-forming carbon atoms, or a substituted or unsubstituted heteroarylene group having 2 to 30 ring-forming carbon atoms.

The compound represented by Formula H-1 may be a monoamine compound. In some embodiments, the compound represented by Formula H-1 may be a diamine compound in which at least one selected from among Arₐ to Ar_{c} includes an amine group as a substituent. In some embodiments, the compound represented by Formula H-1 may be a carbazole-based compound including a substituted or unsubstituted carbazole group in at least one of Arₐ or Ar_{b}, or a fluorene-based compound including a substituted or unsubstituted fluorene group in at least one of Arₐ or Ar_{b}.

The compound represented by Formula H-1 may be represented by any one selected from among the compounds of Compound Group H. However, the compounds listed in Compound Group H are merely examples, and the compounds represented by Formula H-1 are not limited to those represented by Compound Group H:

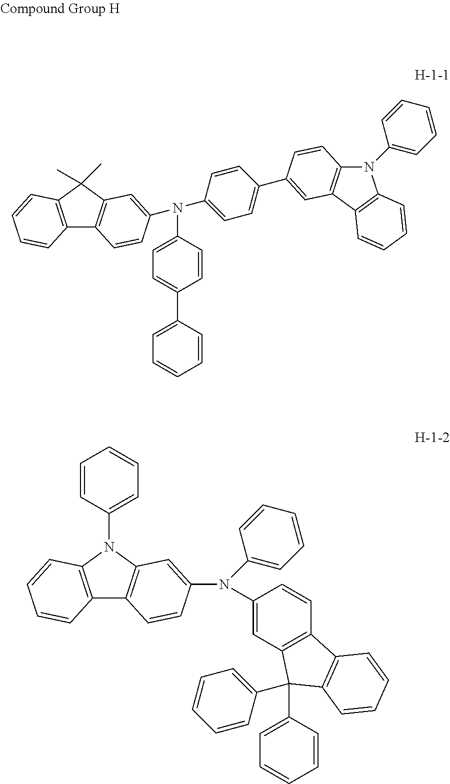 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1]

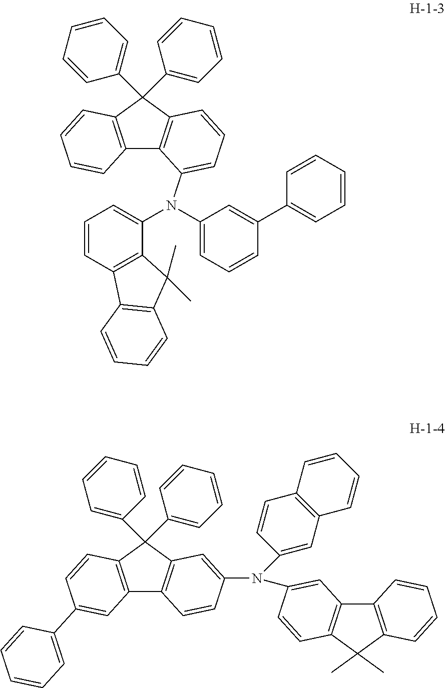 [SMILES: CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

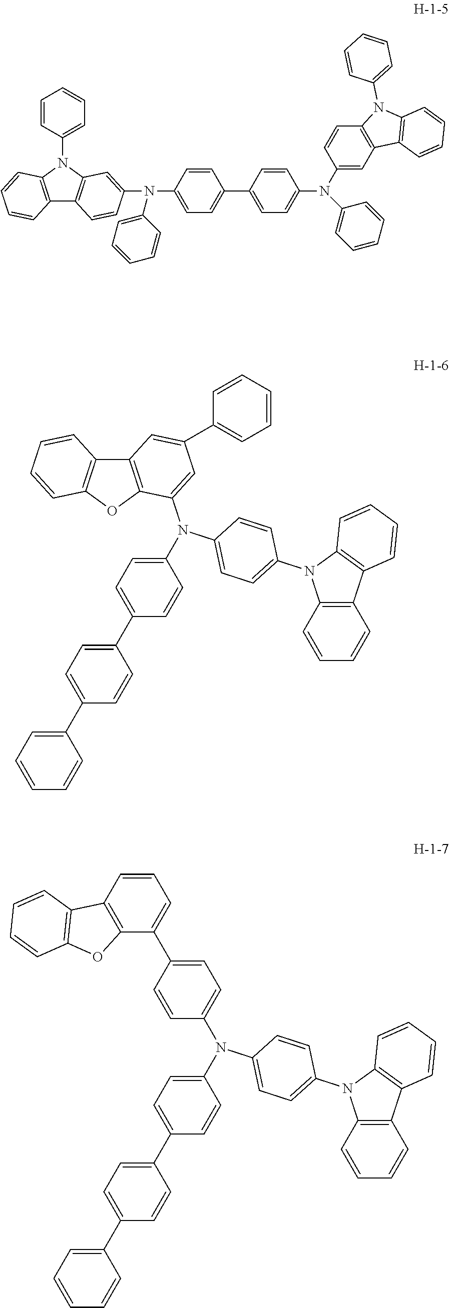 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

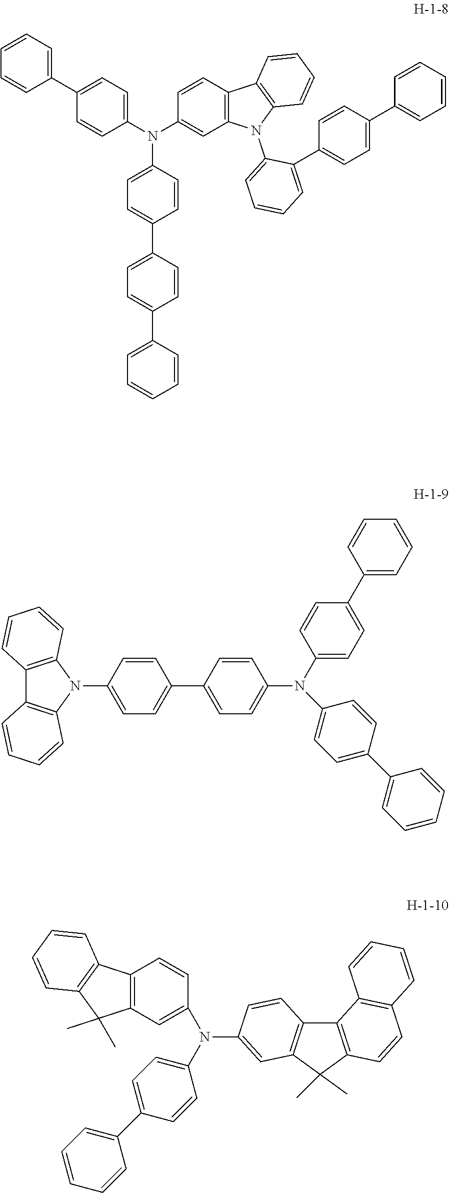 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

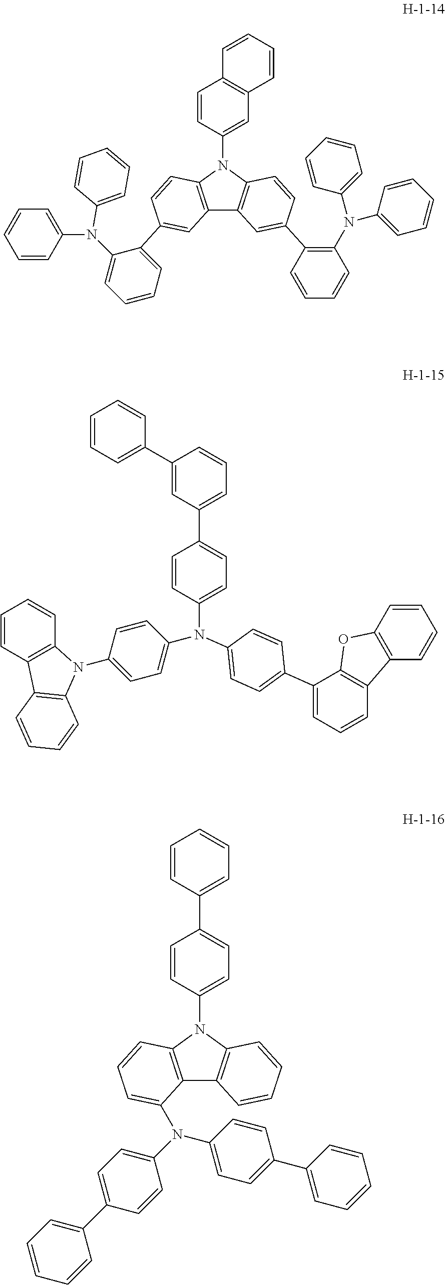 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

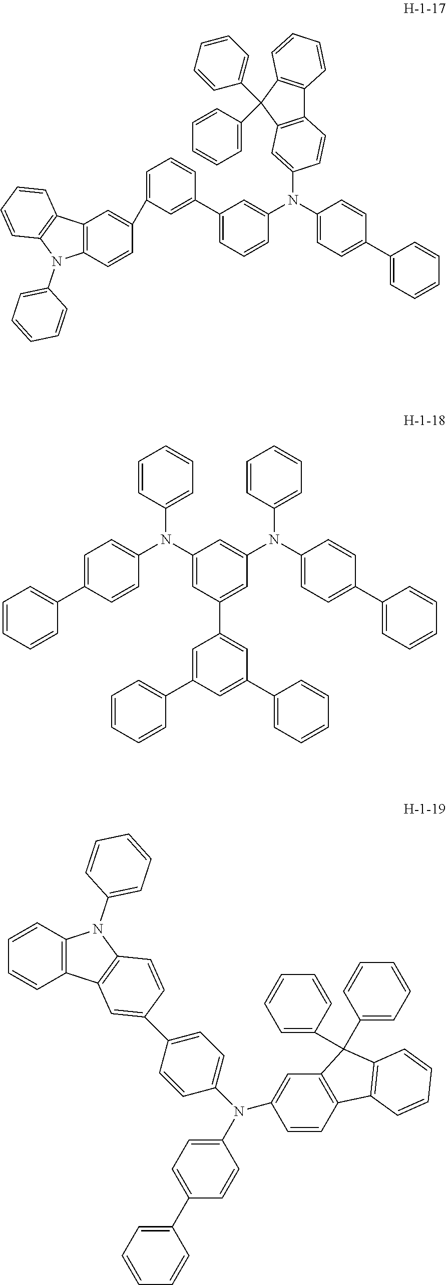 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In some embodiments, the hole transport region HTR may further include a generally used/generally available hole transport material.

For example, the hole transport region HTR may include a phthalocyanine compound such as copper phthalocyanine; N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

The hole transport region HTR may include a carbazole-based derivative such as N-phenyl carbazole or polyvinyl carbazole, a fluorene-based derivative, a triphenylamine-based derivative such as N,N′-bis(3-methylphenyl)-N,N′-diphenyl-[1,1-biphenyl]-4,4′-diamine (TPD) or 4,4′,4″-tris(N-carbazolyl)triphenylamine (TCTA), N,N′-di(naphthalene-I-yl)-N,N′-diphenyl-benzidine (NPB), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 1,3-bis(N-carbazolyl)benzene (mCP), etc.

In some embodiments, the hole transport region HTR may further include 9-(4-tert-butylphenyl)-3,6-bis(triphenylsilyl)-9H-carbazole (CzSi), 9-phenyl-9H-3,9′-bicarbazole (CCP), 1,3-bis(N-carbazolyl)benzene (mCP), 1,3-bis(1,8-dimethyl-9H-carbazol-9-yl)benzene (mDCP), etc.

The hole transport region HTR may include the above-described compounds of the hole transport region in at least one of a hole injection layer HIL, a hole transport layer HTL, or an electron blocking layer EBL.

The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 250 Å to about 1,000 Å. For example, when the hole transport region HTR includes the electron blocking layer EBL, the electron blocking layer EBL may have a thickness of about 10 Å to about 1,000 Å. When the thicknesses of the hole transport region HTR, the hole injection layer HIL, the hole transport layer HTL and the electron blocking layer EBL satisfy the above-described ranges, satisfactory (suitable) hole transport properties may be achieved without a substantial increase in driving voltage.

The hole transport region HTR may further include a charge generating material to increase conductivity in addition to the above-described materials. The charge generating material may be dispersed substantially uniformly or non-uniformly in the hole transport region HTR. The charge generating material may be, for example, a p-dopant. The p-dopant may include at least one of a halogenated metal compound, a quinone derivative, a metal oxide, or a cyano group-containing compound, but the embodiment of the present disclosure is not limited thereto. For example, the p-dopant may include a metal halide compound such as CuI or RbI, a quinone derivative such as tetracyanoquinodimethane (TCNQ) or 2,3,5,6-tetrafluoro-7,7, 8,8-tetracyanoquinodimethane (F4-TCNQ), a metal oxide such as tungsten oxide or molybdenum oxide, a cyano group-containing compound such as dipyrazino[2,3-f: 2′,3′-h] quinoxaline-2,3,6,7,10,11-hexacarbonitrile (HATCN) or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but the embodiment of the present disclosure is not limited thereto.

As described above, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the emission layer EML and may thus increase light emission efficiency. A material that may be contained in the hole transport region HTR may be used as a material to be contained in the buffer layer. The electron blocking layer EBL is a layer that serves to prevent (reduce) the electron injection from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed of a single material, a single layer formed of a plurality of different materials, or a multilayer structure having a plurality of layers formed of a plurality of different materials.

The emission layer EML in the light emitting element ED of an embodiment may emit blue light. The light emitting element ED of an embodiment may include the above-described amine compound of an embodiment in the hole transport region HTR, thereby exhibiting a low driving voltage characteristic in the blue light emitting region. However, the embodiment of the present disclosure is not limited thereto.

In the light emitting element ED of an embodiment, the emission layer EML may include an anthracene derivative, a pyrene derivative, a fluoranthene derivative, a chrysene derivative, a dehydrobenzanthracene derivative, or a triphenylene derivative. For example, the emission layer EML may include the anthracene derivative or the pyrene derivative.

In each of the light emitting elements ED of embodiments illustrated in FIGS. 3 to 6, the emission layer EML may include a host and a dopant, and the emission layer EML may include a compound represented by Formula E-1. The compound represented by Formula E-1 may be used as a fluorescent host material.

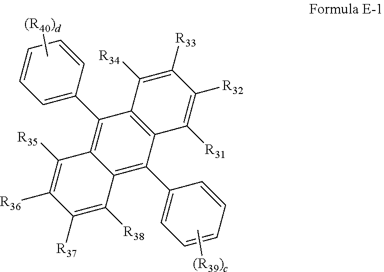

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In some embodiments, R₃₁ to R₄₀ may be bonded to an adjacent group to form a saturated hydrocarbon ring or an unsaturated hydrocarbon ring, a saturated heterocycle, or an unsaturated heterocycle.

In Formula E-1, c and d may each independently be an integer from 0 to 5.

Formula E-1 may be represented by any one selected from among Compound E1 to Compound E19:

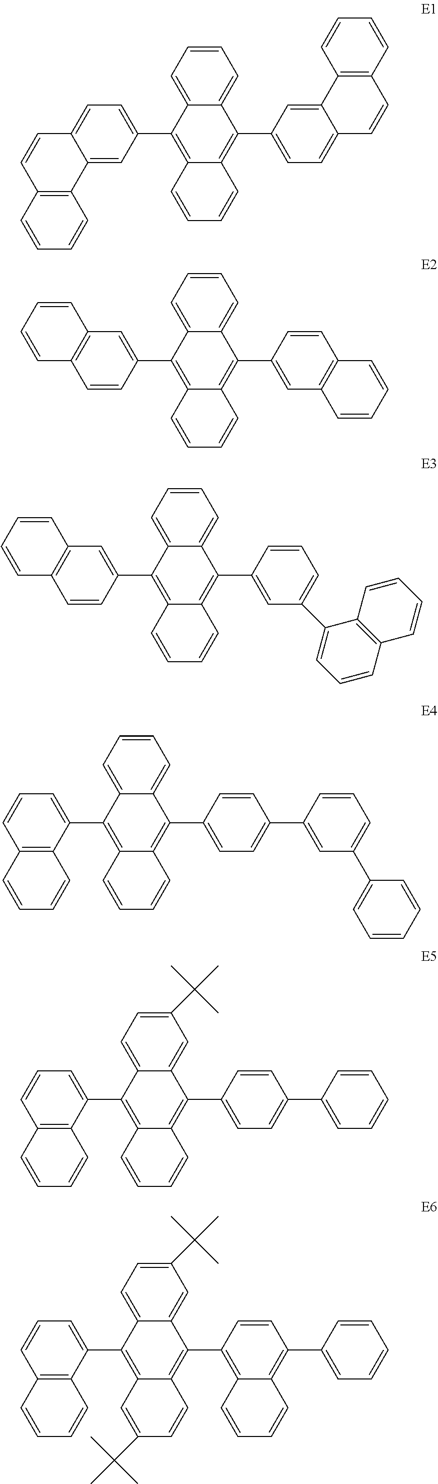

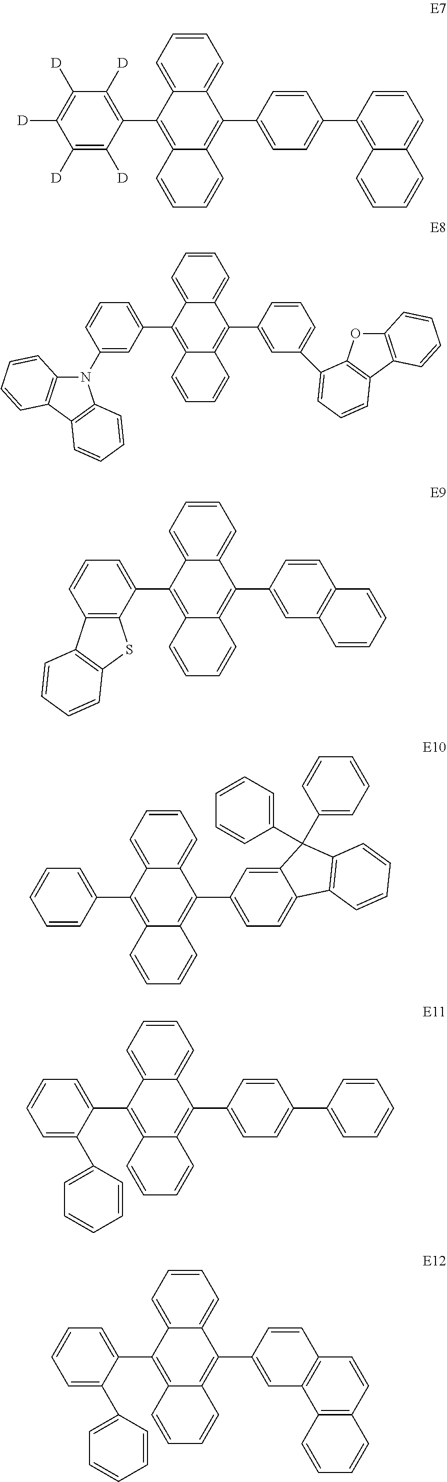

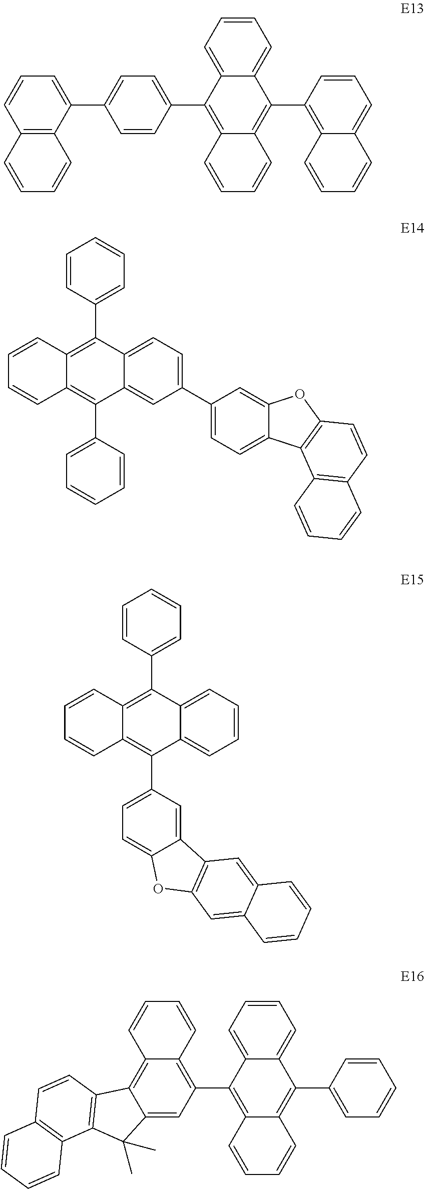

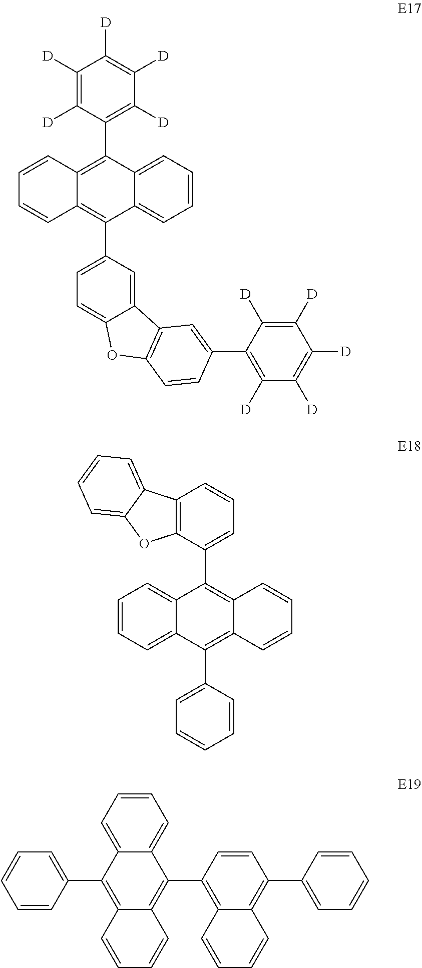

In an embodiment, the emission layer EML may include a compound represented by Formula E-2a or Formula E-2b. The compound represented by Formula E-2a or Formula E-2b may be used as a phosphorescent host material.

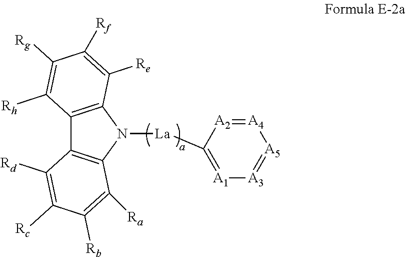

In Formula E-2a, a may be an integer from 0 to 10, Lₐ may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or more, a plurality of Las may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may each independently be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Rₐ to R may be bonded to an adjacent group to form a hydrocarbon ring or a heterocycle containing N, O, S, etc. as a ring-forming atom.

In some embodiments, in Formula E-2a, two or three selected from among A₁ to A₅ may be N, and the rest (those that are not N) may be CRᵢ.

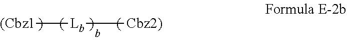

In Formula E-2b, Cbz1 and Cbz2 may each independently be an unsubstituted carbazole group, or a carbazole group substituted with an aryl group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, b may be an integer from 0 to 10, and when b is an integer of 2 or more, a plurality of L_{b}s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one selected from among the compounds of Compound Group E-2. However, the compounds listed in Compound Group E-2 are merely examples, and the compound represented by Formula E-2a or Formula E-2b is not limited to those represented in Compound Group E-2.

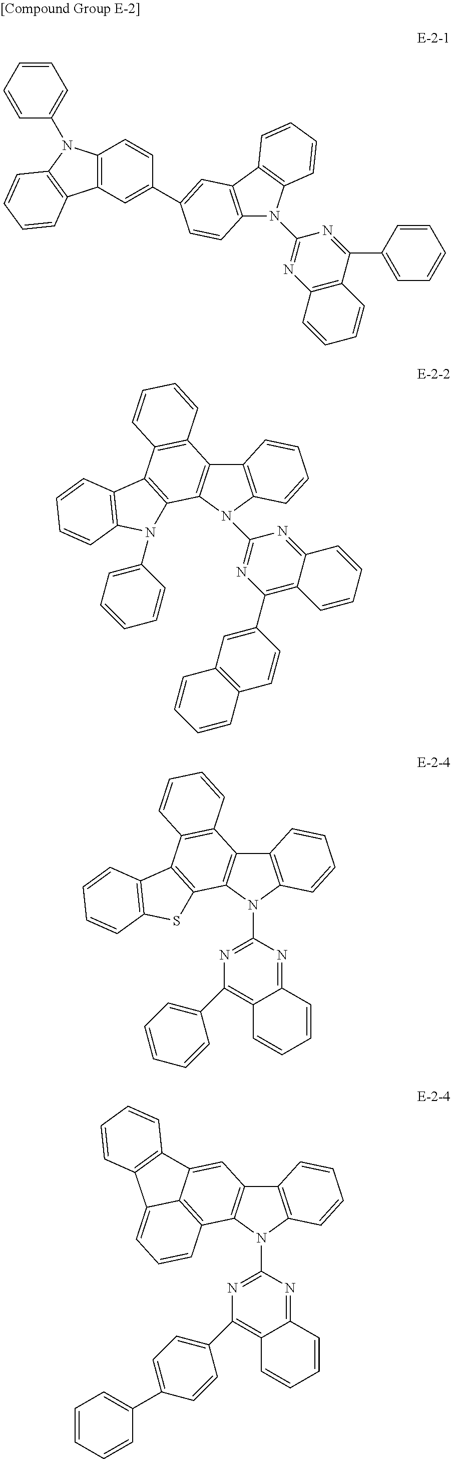

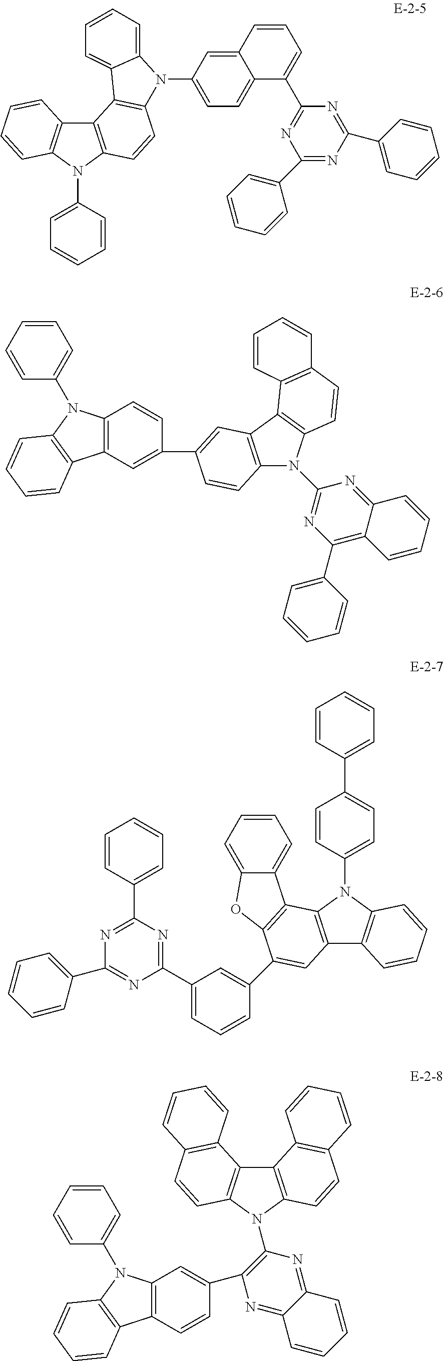 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

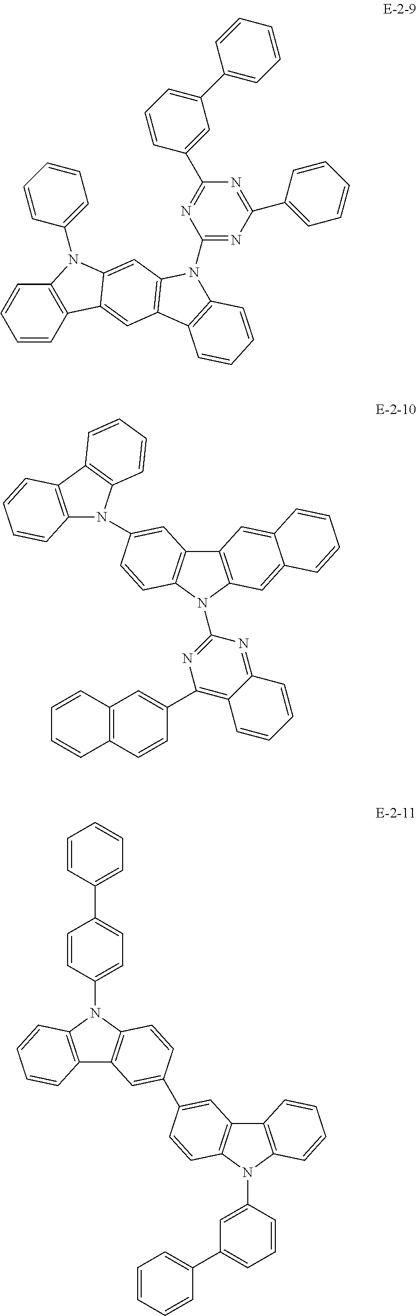 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

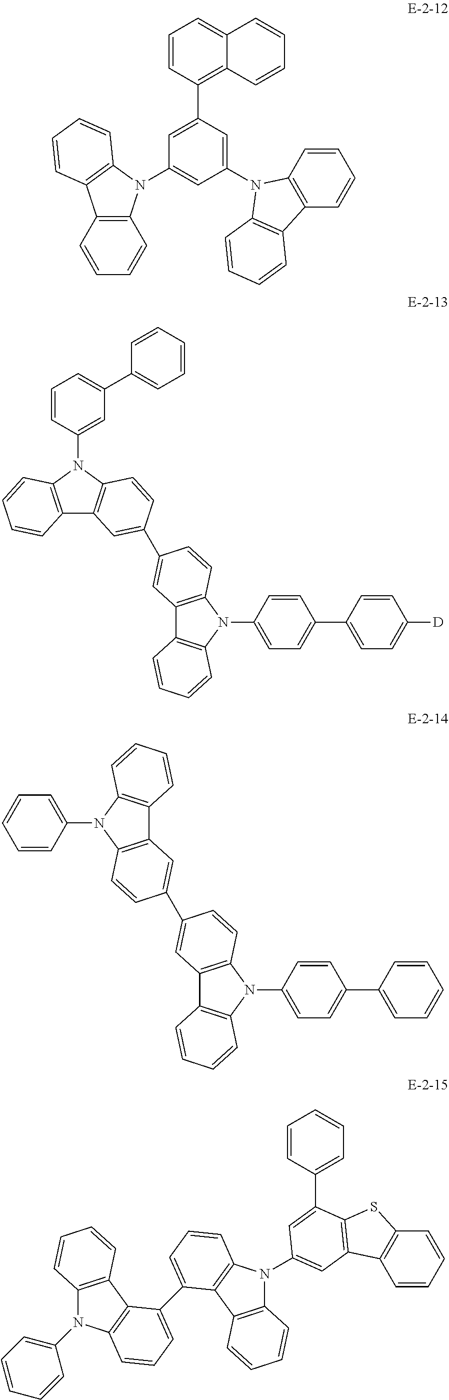 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

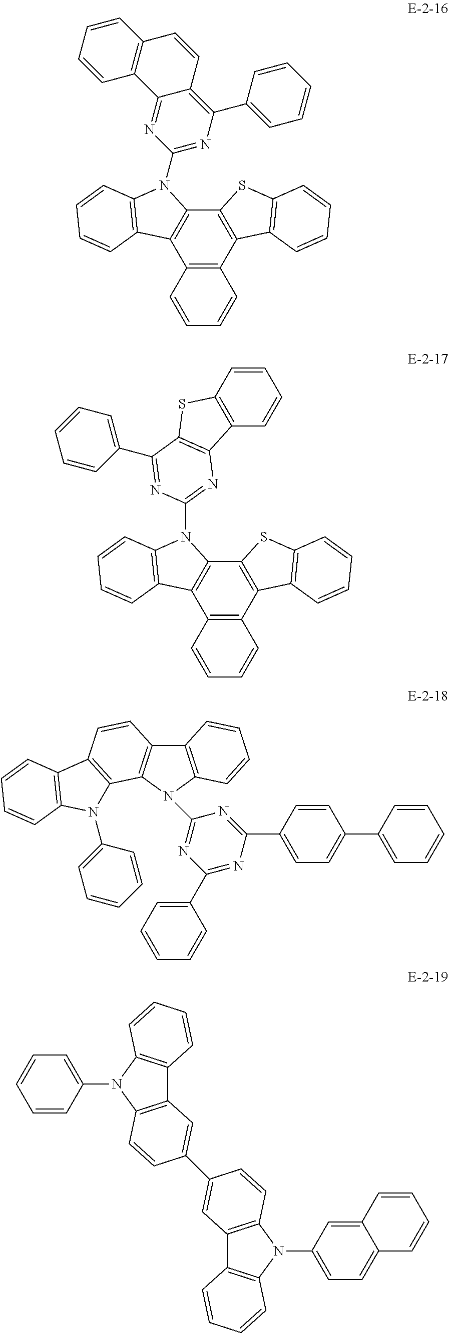

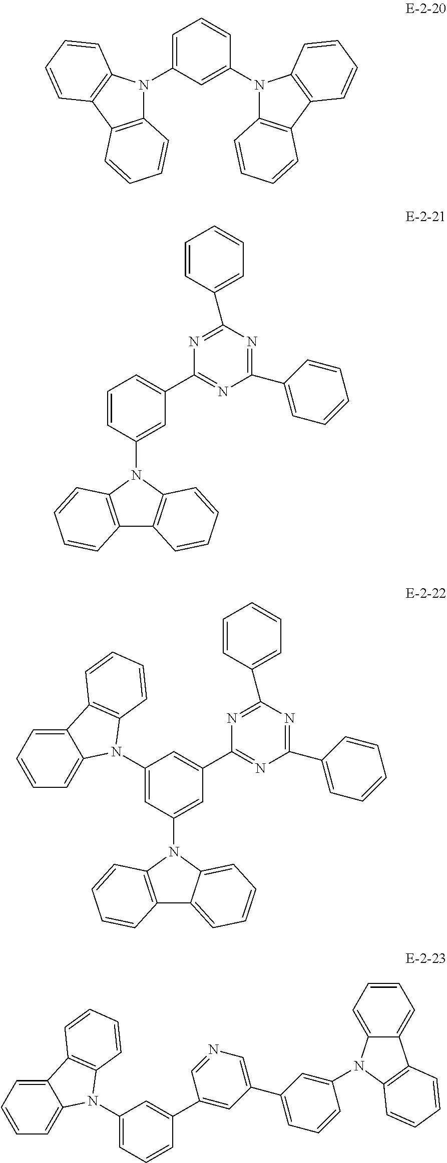 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

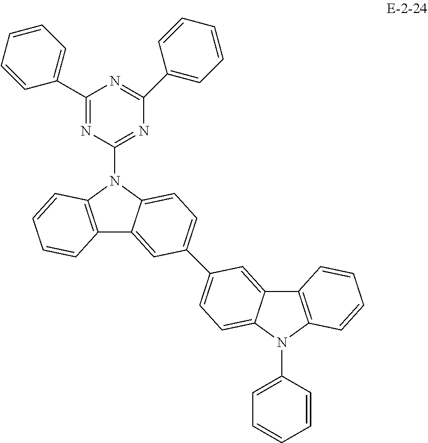 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a material generally used/generally available in the art as a host material. For example, the emission layer EML may include, as a host material, at least one of bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, the embodiment of the present disclosure is not limited thereto, for example, tris(8-hydroxyquinolino)aluminum (Alq₃), 9,10-di(naphthalene yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as a host material.

The emission layer EML may include a compound represented by Formula M-a or Formula M-b. The compound represented by Formula M-a or Formula M-b may be used as a phosphorescent dopant material.

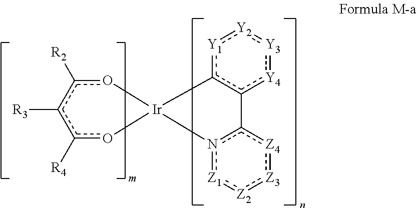

In Formula M-a, Y₁ to Y₄ and Z₁ to Z₄ may each independently be CR₁ or N, R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula M-a, m may be 0 or 1, and n may be 2 or 3. In Formula M-a, when m is 0, n is 3, and when m is 1, n is 2.

The compound represented by Formula M-a may be used as a phosphorescent dopant.

The compound represented by Formula M-a may be represented by any one selected from among Compound M-a1 to Compound M-a25. However, Compounds M-a1 to M-a25 are merely examples, and the compound represented by Formula M-a is not limited to those represented by Compounds M-a1 to M-a25.

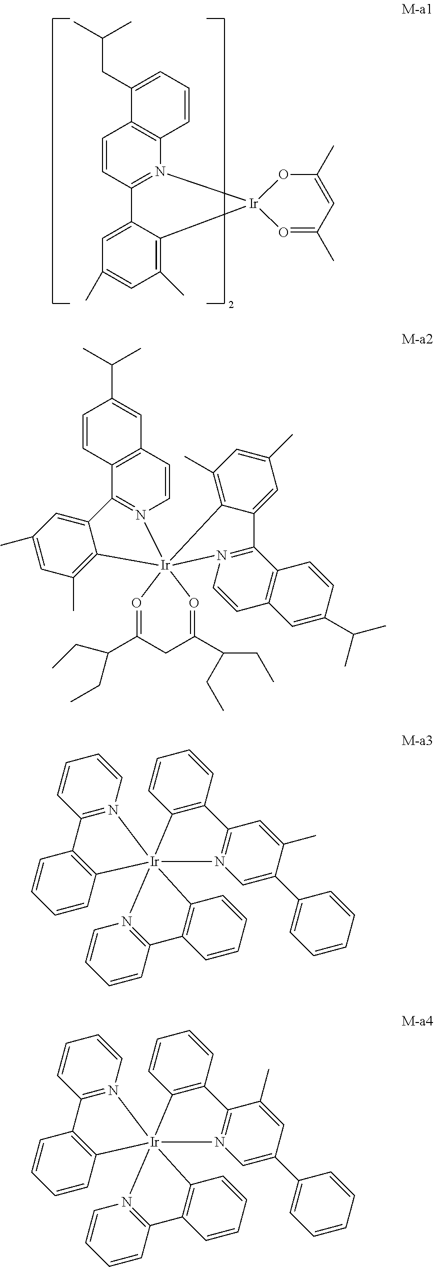

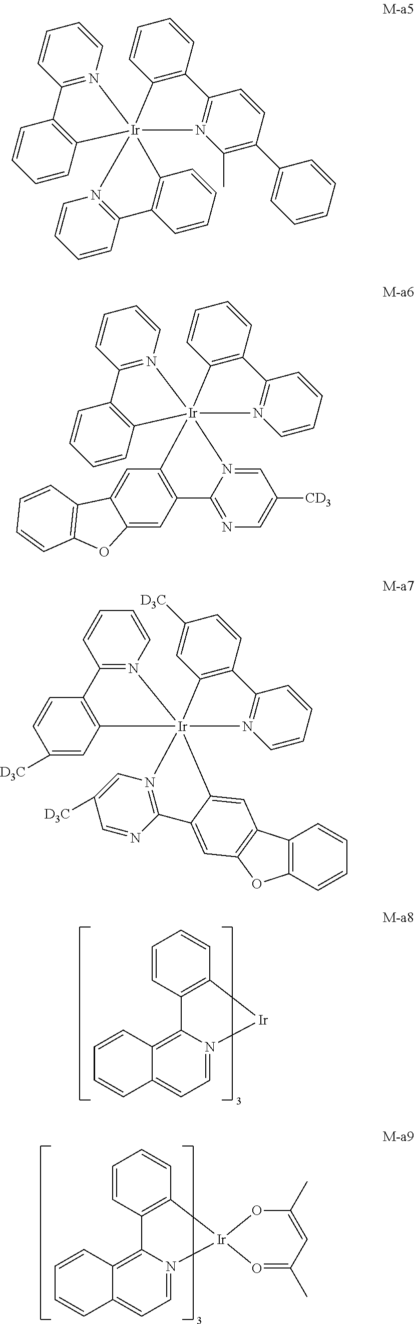

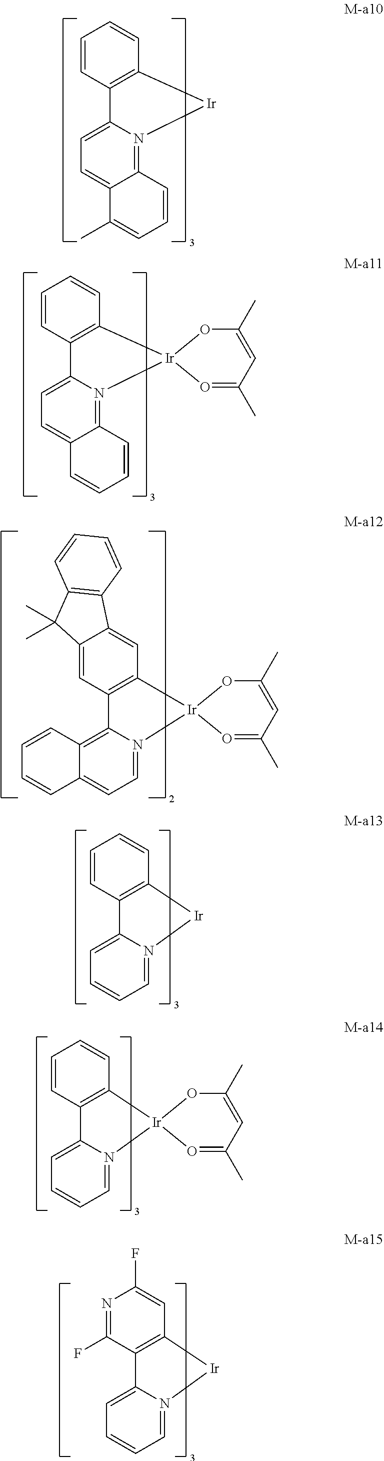

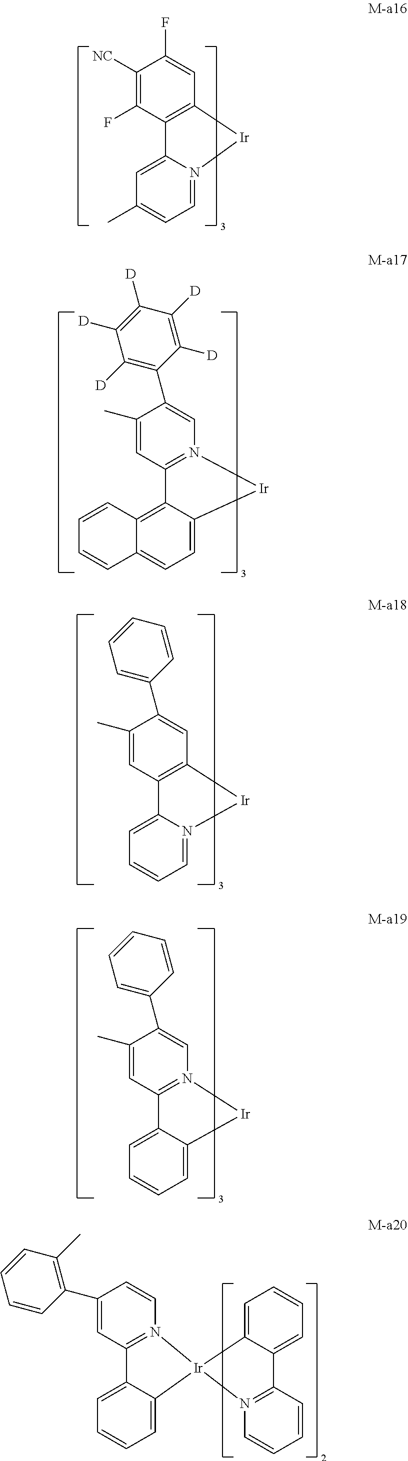

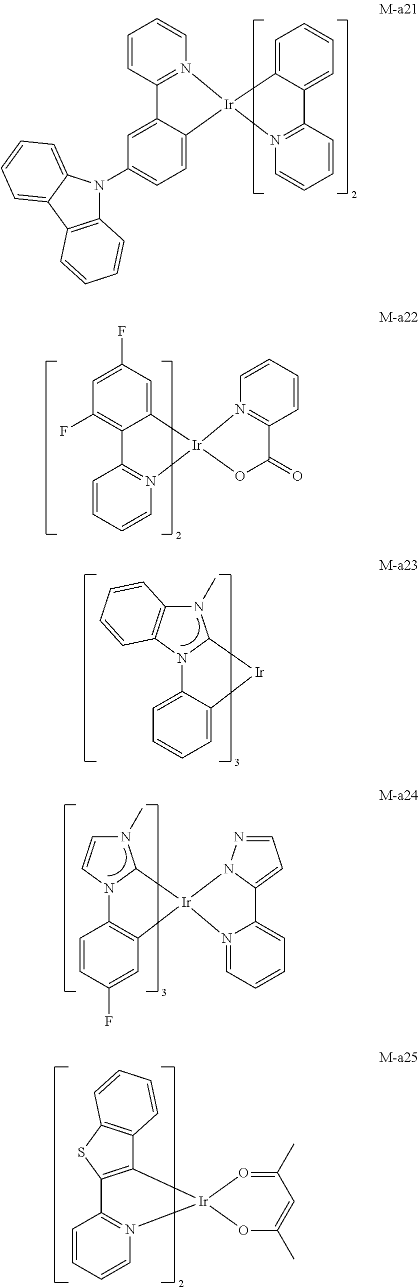

Compound M-a1 and Compound M-a2 may be used as a red dopant material, and Compound M-a3 to Compound M-a7 may be used as a green dopant material.

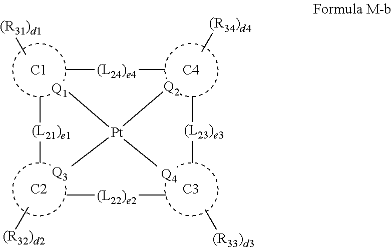

In Formula M-b, Q₁ to Q₄ may each independently be C or N, and C1 to C4 may each independently be a substituted or unsubstituted hydrocarbon ring having 5 to 30 ring-forming carbon atoms, or a substituted or unsubstituted heterocycle having 2 to 30 ring-forming carbon atoms. L₂₁ to L₂₄ may each independently be a direct linkage,

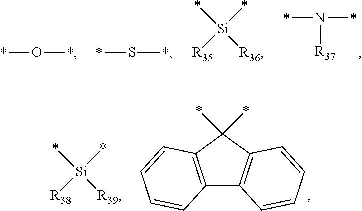

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring, and d1 to d4 may each independently be an integer from 0 to 4.

The compound represented by Formula M-b may be used as a blue phosphorescent dopant or a green phosphorescent dopant.

The compound represented by Formula M-b may be represented by any one selected from among Compounds M-b-1 to M-b-12. However, the following compounds are merely examples, and the compounds represented by Formula M-b are not limited to Compounds M-b-1 to M-b-12:

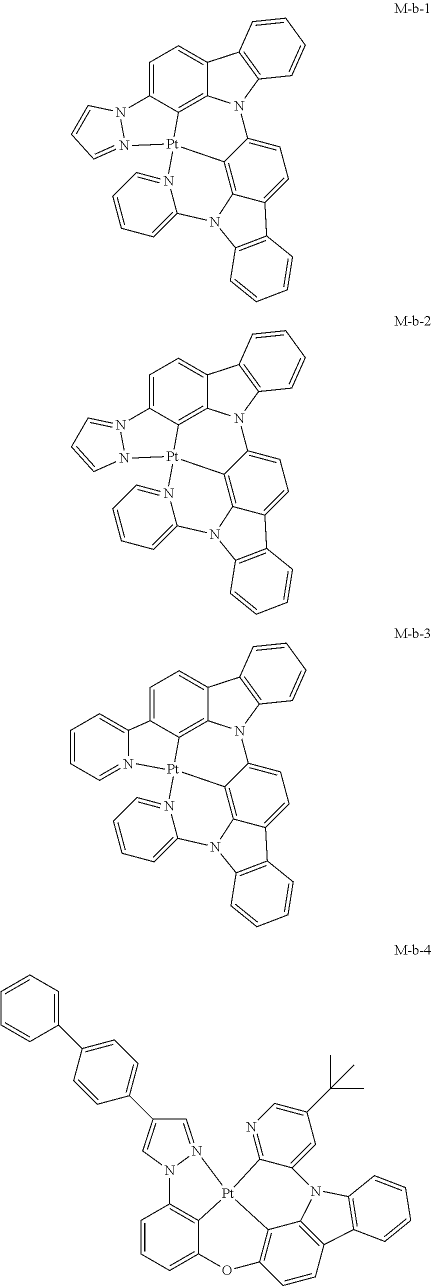

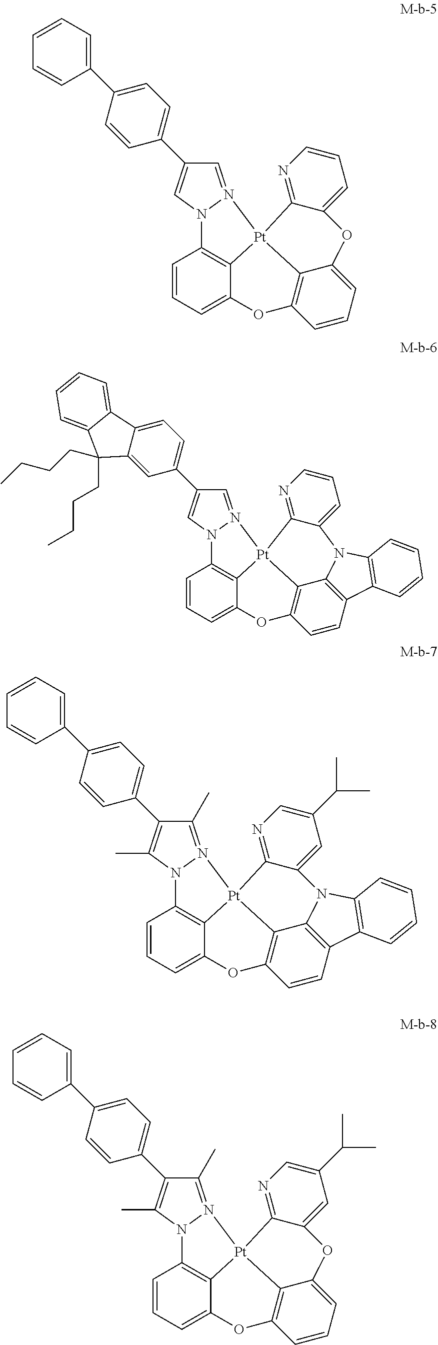

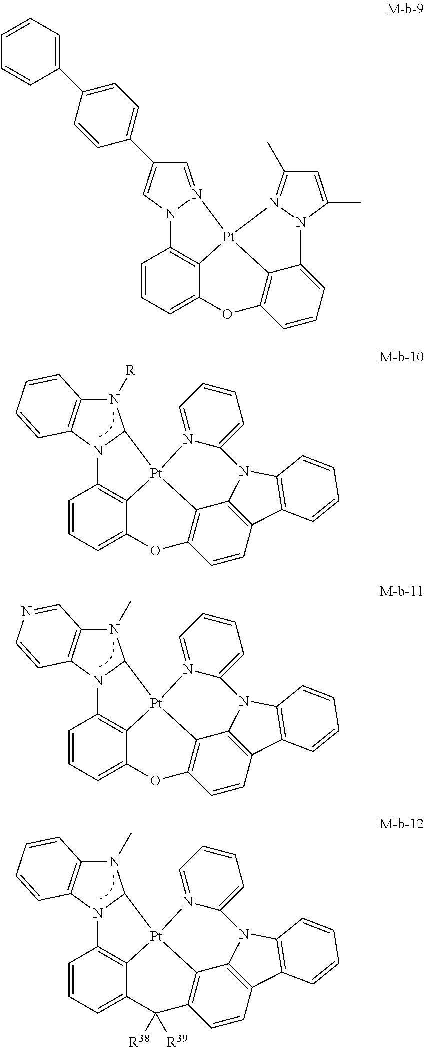

In the compounds, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may include a compound represented by any one among Formula F-a to Formula F-c. The compound represented by Formula F-a or Formula F-c may be used as a fluorescence dopant material.

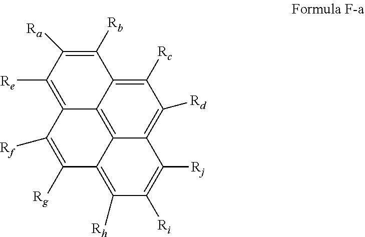

In Formula F-a, two selected from among Rₐ to Rⱼ may each independently be substituted with *—NAr₁Ar₂. The others, which are not substituted with *—NAr₁Ar₂, among Rₐ to Rⱼ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In *—NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

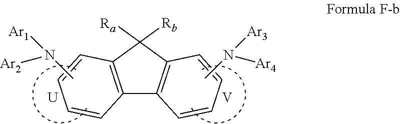

In Formula F-b, Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms.

In Formula F-b, the number of rings represented by U and V may each independently be 0 or 1. For example, in Formula F-b, when the number of U or V is 1, one ring constitutes a fused ring at a portion indicated by U or V, and when the number of U or V is 0, a ring indicated by U or V does not exist. For example, when the number of U is 0 and the number of V is 1, or when the number of U is 1 and the number of V is 0, the fused ring having a fluorene core in Formula F-b may be a cyclic compound having four rings. In some embodiments, when each number of U and V is 0, the fused ring in Formula F-b may be a cyclic compound having three rings. In some embodiments, when each number of U and V is 1, the fused ring having a fluorene core in Formula F-b may be a cyclic compound having five rings.

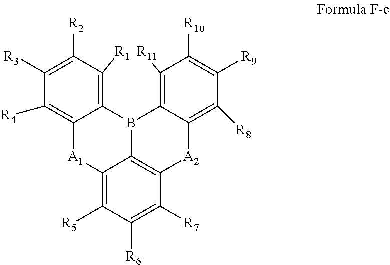

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring.

In Formula F-c, A₁ and A₂ may each independently be bonded to substituents of an adjacent ring to form a condensed ring. For example, when A₁ and A₂ may each independently be NRₘ, A₁ may be bonded to R₄ or R₅ to form a ring. In some embodiments, A₂ may be bonded to R₇ or R₈ to form a ring.

In an embodiment, the emission layer EML may include, as a generally used/generally available dopant material, a styryl derivative (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), and N-(4-((E)-2-(6-((E) (diphenylamino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi), perylene and a derivative thereof (e.g., 2,5,8,11-tetra-t-butylperylene (TBP)), pyrene and a derivative thereof (e.g., 1,1-dipyrene, 1,4-dipyrenylbenzene, 1,4-bis(N,N-diphenylamino)pyrene), etc.

The emission layer EML may include a generally used/generally available phosphorescent dopant material. For example, a metal complex containing iridium (Ir), platinum (Pt), osmium (Os), aurum (Au), titanium (Ti), zirconium (Zr), hafnium (Hf), europium (Eu), terbium (Tb), or thulium (Tm) may be used as a phosphorescent dopant. For example, iridium(III) bis(4,6-difluorophenylpyridinato-N,C2′)picolinate (Flrpic), bis(2,4-difluorophenylpyridinato)-tetrakis(1-pyrazolyl)borate iridium(III) (Fir₆), or platinum octaethyl porphyrin (PtOEP) may be used as a phosphorescent dopant. However, the embodiment of the present disclosure is not limited thereto.

In some embodiments, the emission layer EML may include a hole transport host and an electron transport host. In some embodiments, the emission layer EML may include an auxiliary dopant and a light emitting dopant. In some embodiments, a phosphorescent dopant material or a thermally delayed fluorescent dopant material may be included as the auxiliary dopant. For example, the emission layer EML in an embodiment may include the hole transport host, the electron transport host, the auxiliary dopant, and the light emitting dopant.

In some embodiments, in the emission layer EML, an exciplex may be formed by the hole transport host and the electron transport host. In this embodiment, a triplet energy of the exciplex formed by the hole transport host and the electron transport host may correspond to T1 that is a gap between a lowest unoccupied molecular orbital (LUMO) energy level of the electron transport host and a HOMO energy level of the hole transport host.

In an embodiment, the triplet energy (T1) of the exciplex formed by the hole transport host and the electron transport host may be about 2.4 eV to about 3.0 eV. In some embodiments, the triplet energy of the exciplex may be a value smaller than an energy gap of each host material. Therefore, the exciplex may have a triplet energy of about 3.0 eV or less that is an energy gap between the hole transport host and the electron transport host.

In some embodiments, at least one emission layer EML may include a quantum dot material. The core of the quantum dot may be selected from among a Group II-VI compound, a Group III-VI compound, a Group I-III-IV compound, a Group III-V compound, a Group III-II-V compound, a Group IV-VI compound, a Group IV element, a Group IV compound, and one or more combinations thereof.

The Group II-VI compound may be selected from the group including (e.g., consisting of) a binary compound selected from the group including (e.g., consisting of) CdSe, CdTe, CdS, ZnS, ZnSe, ZnTe, ZnO, HgS, HgSe, HgTe, MgSe, MgS, and one or more compounds or mixtures thereof, a ternary compound selected from the group including (e.g., consisting of) CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and one or more compounds or mixtures thereof, and a quaternary compound selected from the group including (e.g., consisting of) HgZnTeS, CdZnSeS, CdZnSeTe, CdZnSTe, CdHgSeS, CdHgSeTe, CdHgSTe, HgZnSeS, HgZnSeTe, HgZnSTe, and one or more compounds or mixtures thereof.

The Group III-VI compound may include a binary compound such as In₂S₃ or In₂Se₃, a ternary compound such as InGaS₃ or InGaSe₃, or one or more combinations thereof.

The Group I-III-VI compound may be selected from a ternary compound selected from the group including (e.g., consisting of) AgInS, AgInS₂, CuInS, CuInS₂, AgGaS₂, CuGaS₂ CuGaO₂, AgGaO₂, AgAlO₂, and one or more compounds or mixtures thereof, and/or a quaternary compound such as AgInGaS₂ or CuInGaS₂ (the quaternary compound may be used alone or in combination with any of the foregoing compounds or mixtures; and the quaternary compound may also be combined with other quaternary compounds).

The Group III-V compound may be selected from the group including (e.g., consisting of) a binary compound selected from the group including (e.g., consisting of) GaN, GaP, GaAs, GaSb, AlN, AlP, AlAs, AlSb, InN, InP, InAs, InSb, and one or more compounds or mixtures, a ternary compound selected from the group including (e.g., consisting of) GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and one or more compounds or mixtures, and a quaternary compound selected from the group including (e.g., consisting of) GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and one or more compounds or mixtures thereof. In some embodiments, the Group III-V compound may further include a Group II metal. For example, InZnP, etc. may be selected as a Group III-II-V compound.

The Group IV-VI compound may be selected from the group including (e.g., consisting of) a binary compound selected from the group including (e.g., consisting of) SnS, SnSe, SnTe, PbS, PbSe, PbTe, and one or more compounds or mixtures thereof, a ternary compound selected from the group including (e.g., consisting of) SnSeS, SnSeTe, SnSTe, PbSeS, PbSeTe, PbSTe, SnPbS, SnPbSe, SnPbTe, and one or more compounds or mixtures thereof, and a quaternary compound selected from the group including (e.g., consisting of) SnPbSSe, SnPbSeTe, SnPbSTe, and one or more compounds or mixtures thereof. The Group IV element may be selected from the group including (e.g., consisting of) Si, Ge, and one or more elements or mixtures thereof. The Group IV compound may be a binary compound selected from the group including (e.g., consisting of) SiC, SiGe, and one or more compounds or mixtures thereof.

In this embodiment, a binary compound, a ternary compound, or a quaternary compound may be present in a particle form and the particle being with a uniform concentration distribution, or may be present in the same particle with a partially different concentration distribution. In some embodiments, a core/shell structure in which one quantum dot surrounds another quantum dot may also be possible. The core/shell structure may have a concentration gradient in which the concentration of elements present in the shell decreases toward the core.

In some embodiments, the quantum dot may have the above-described core/shell structure including a core containing nanocrystals and a shell surrounding the core. The shell of the quantum dot may serve as a protection layer to prevent or reduce the chemical deformation of the core so as to maintain semiconductor properties, and/or a charging layer to impart electrophoresis properties to the quantum dot. The shell may be a single layer or a multilayer. An example of the shell of the quantum dot may include a metal or non-metal oxide, a semiconductor compound, or one or more combinations thereof.

For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and NiO, or a ternary compound such as MgAl₂O₄, CoFe₂O₄, NiFe₂O₄, and/or CoMn₂O₄, but the present disclosure is not limited thereto.

In some embodiments, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the embodiment of the present disclosure is not limited thereto.

The quantum dot may have a full width of half maximum (FWHM) of a light emitting wavelength spectrum of about 45 nm or less, about 40 nm or less, or about 30 nm or less, and color purity or color reproducibility may be improved in the above range. In some embodiments, light emitted through such a quantum dot is emitted in all directions, and thus a wide viewing angle may be improved (increased).

In some embodiments, although the form of a quantum dot is not limited as long as it is a form commonly used in the art, more specifically, a quantum dot in the form of a substantially spherical, pyramidal, multi-arm, or cubic nanoparticles, nanotubes, nanowires, nanofibers, nanoparticles, etc. may be used.

The quantum dot may control the color of emitted light according to the particle size thereof. Accordingly, the quantum dot may have one or more suitable light emission colors such as blue, red, and green.

In each of the light emitting elements ED of embodiments illustrated in FIGS. 3 to 6, the electron transport region ETR is provided on the emission layer EML. The electron transport region ETR may include at least one of the hole blocking layer HBL, the electron transport layer ETL, or the electron injection layer EIL, but the embodiment of the present disclosure is not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in order from the emission layer EML, but the embodiment of the present disclosure is not limited thereto. The electron transport region ETR may have a thickness, for example, from about 1,000 Å to about 1,500 Å.

The electron transport region ETR may be formed by using one or more suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1:

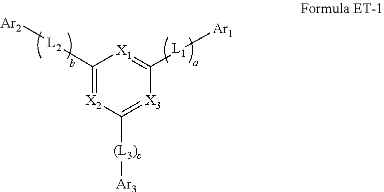

In Formula ET-1, at least one selected from among X₁ to X₃ may be N, and the rest (those that are not N) are CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer from 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a to c are an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the embodiment of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-Bis[3,5-di(pyridin-3-yl)phenyl]benzene (BmPyPhB), or one or more compounds or mixtures thereof.

The electron transport region ETR may include at least one selected from among Compound ET1 to Compound ET36:

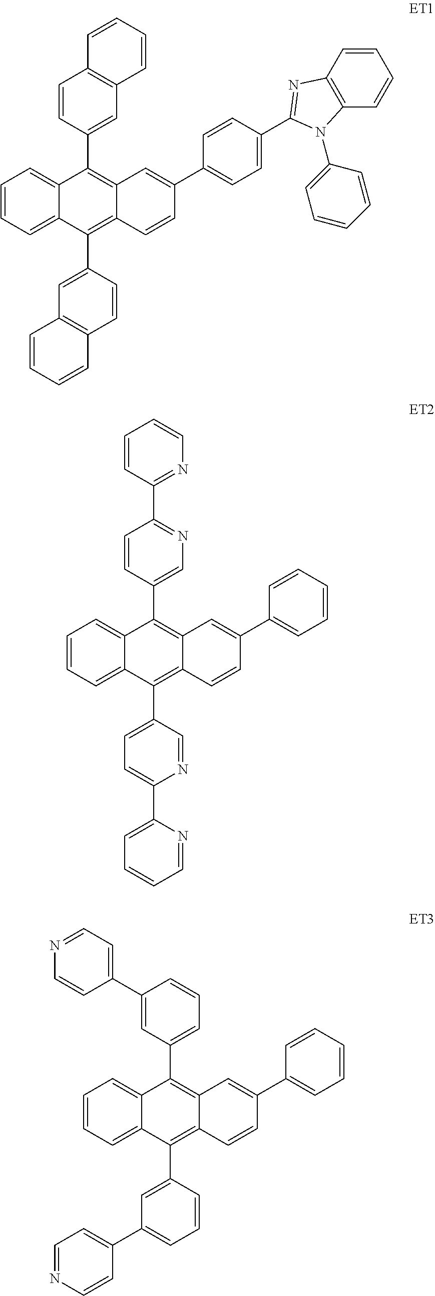 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

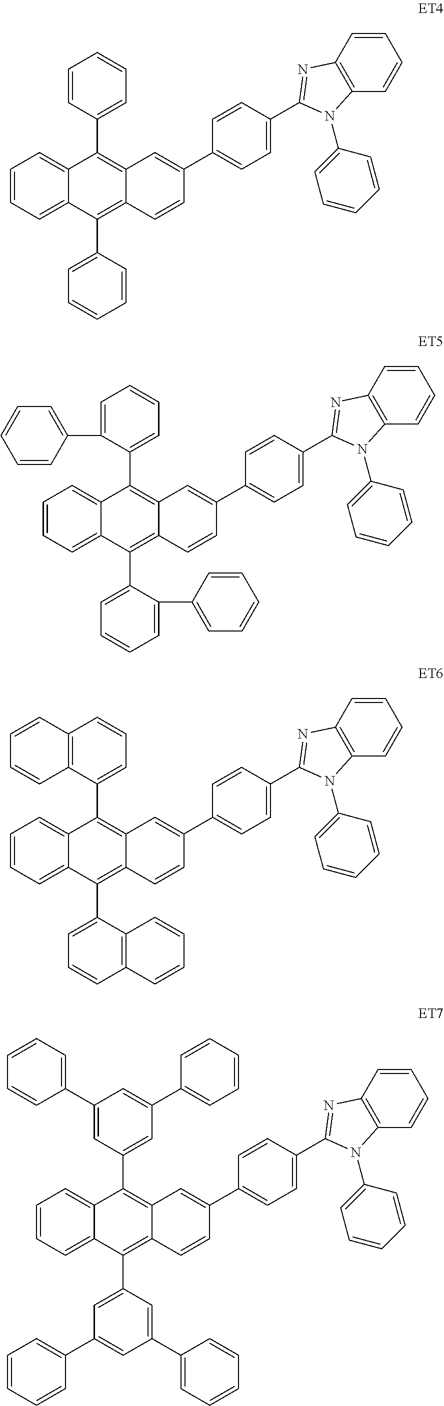 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

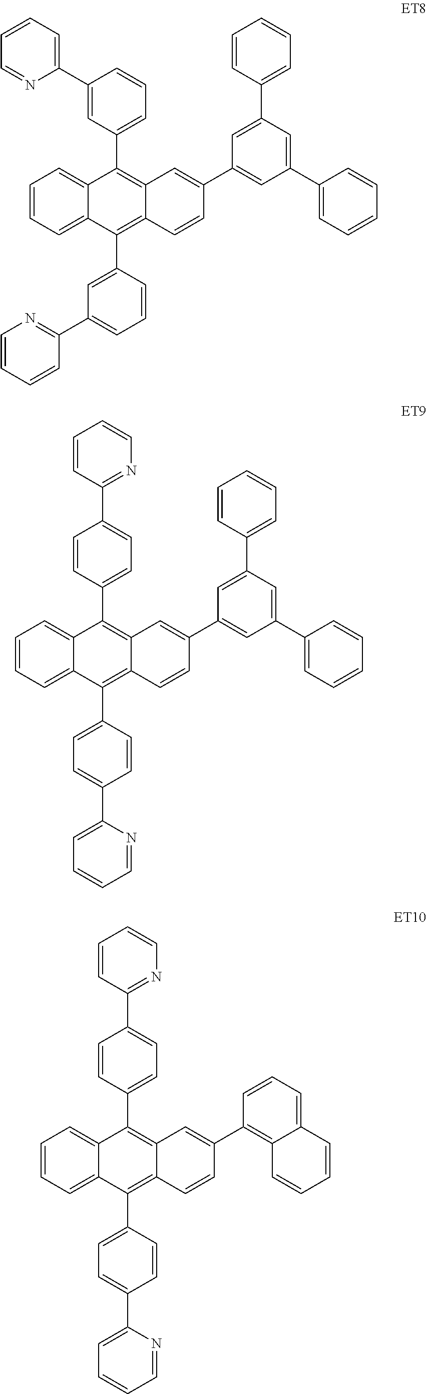 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

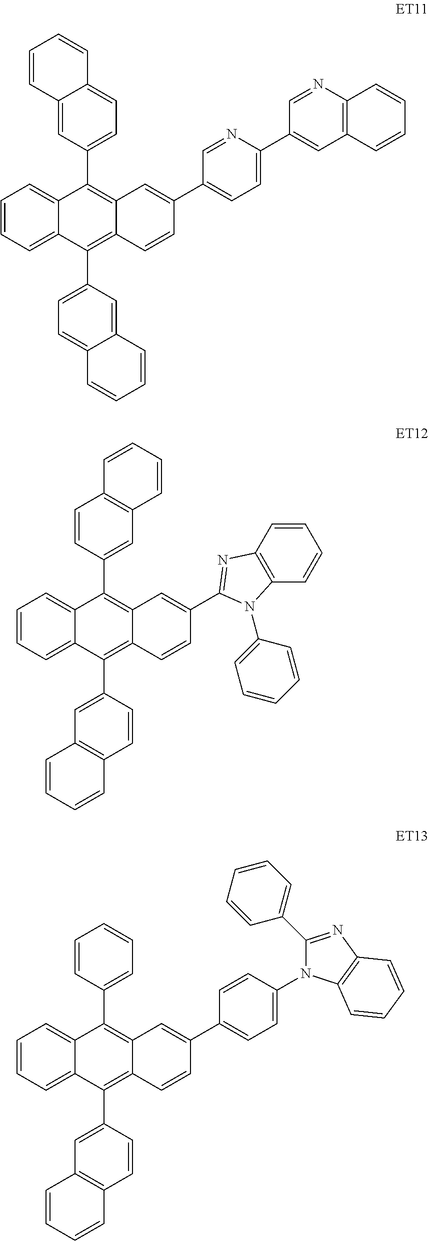

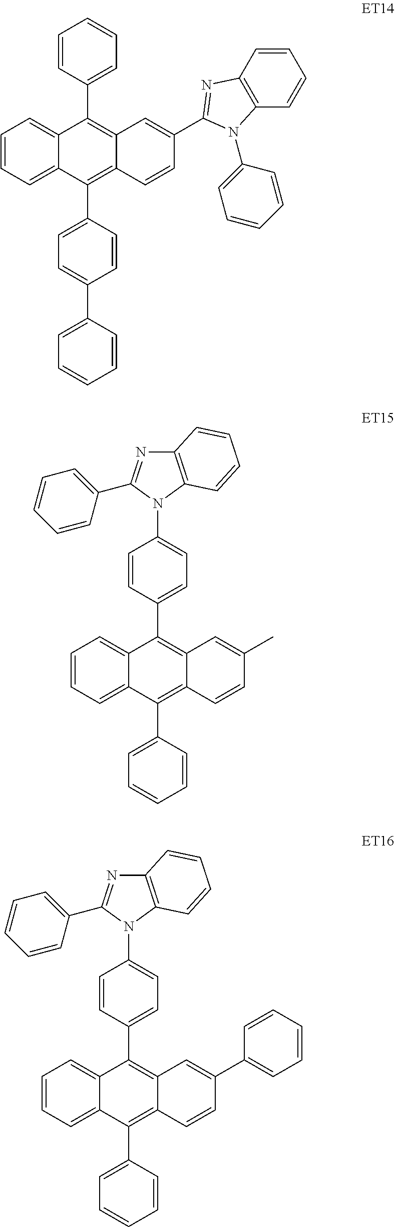 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

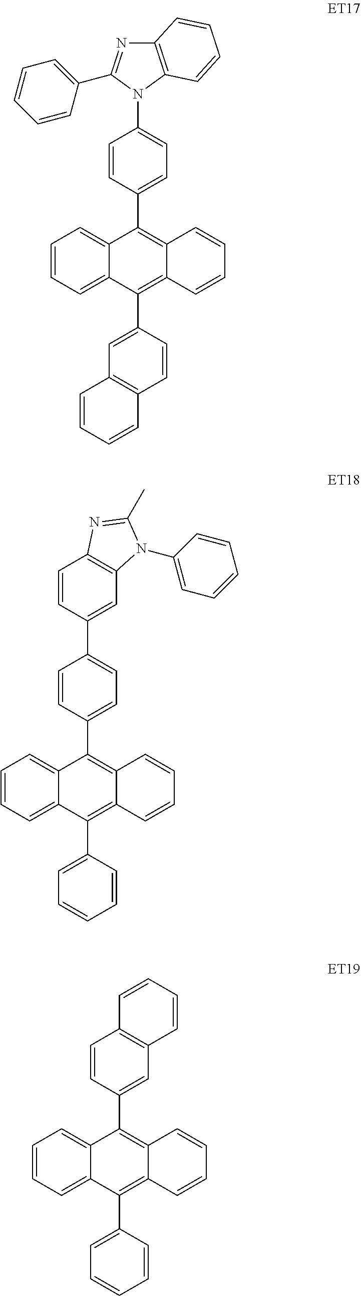

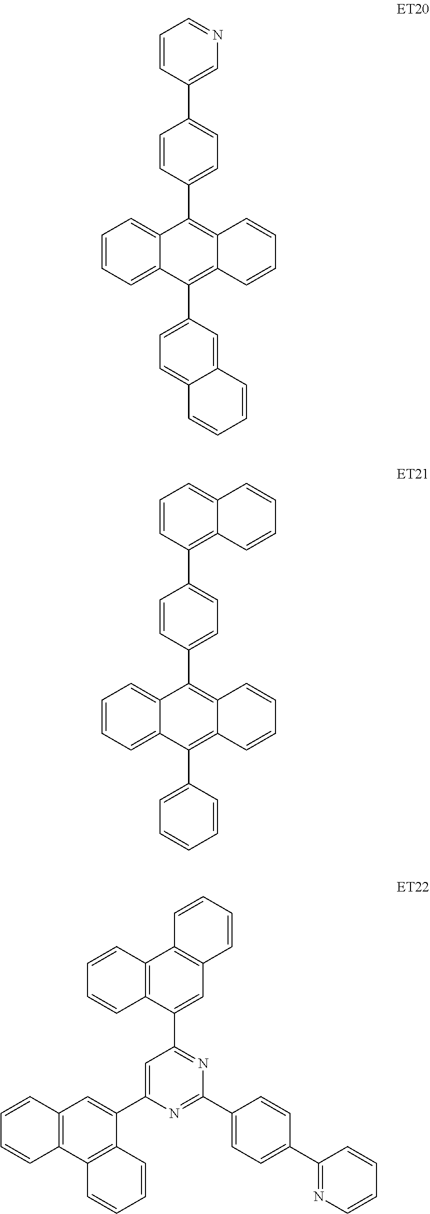

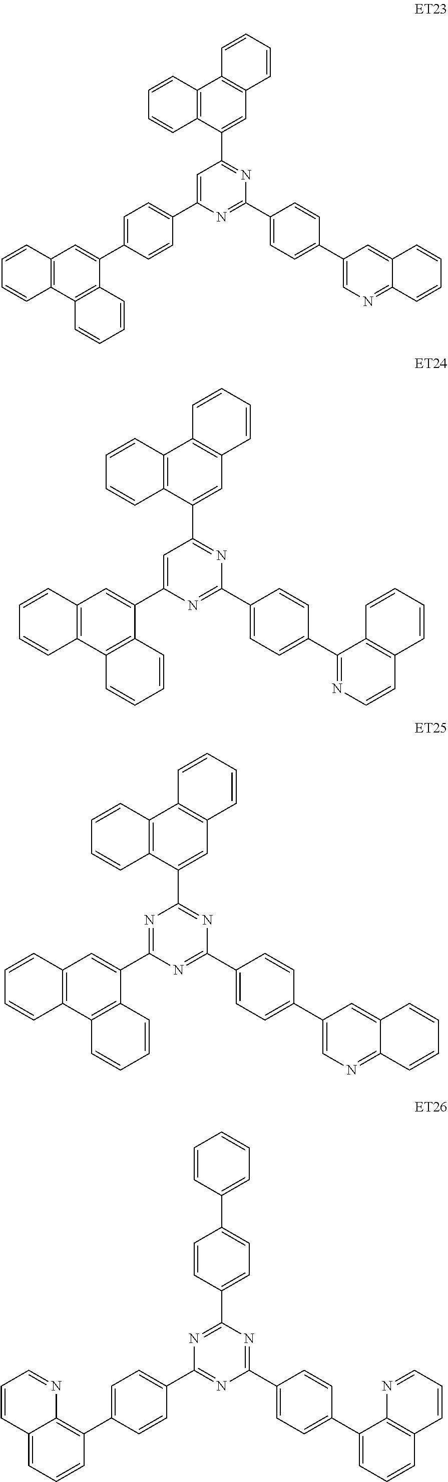 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1]

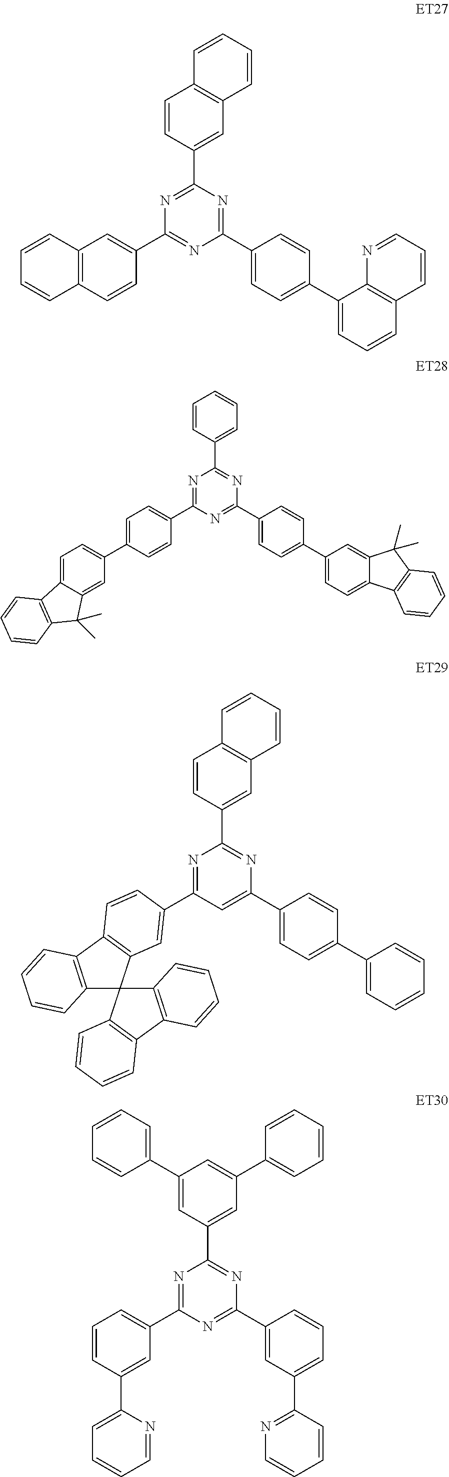

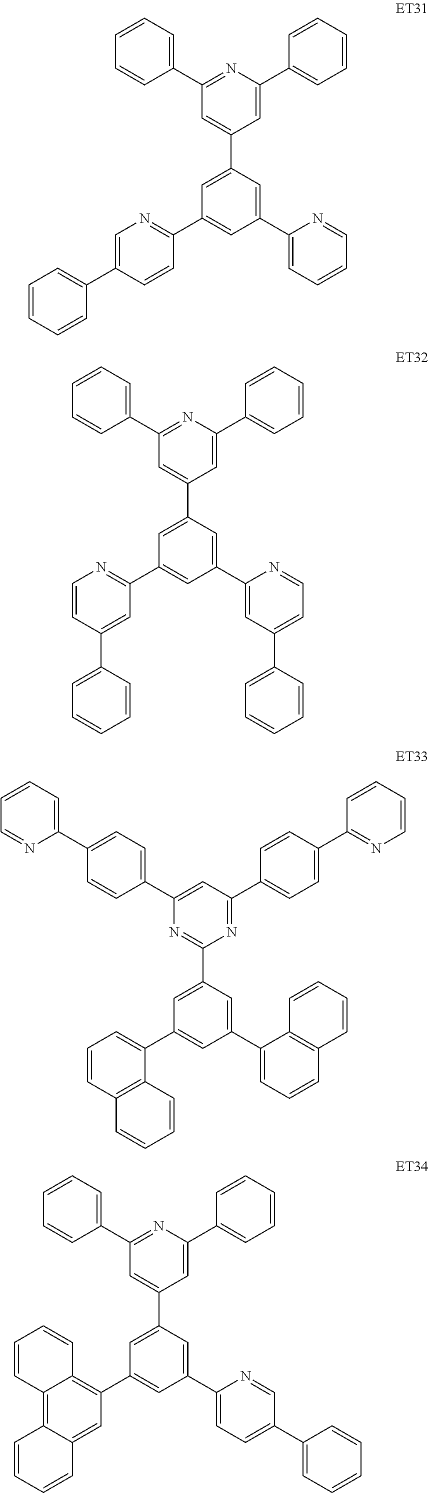 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

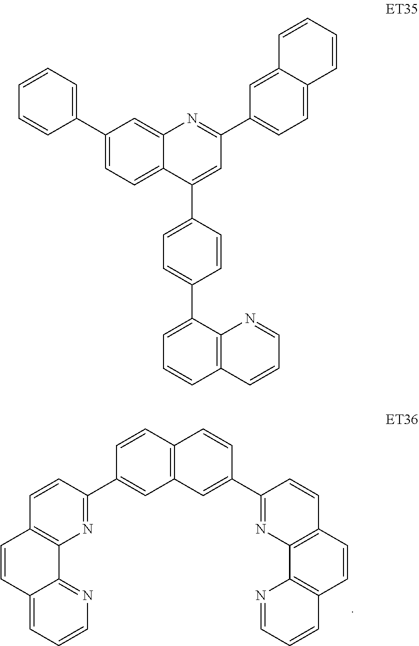

In some embodiments, the electron transport regions ETR may include a metal halide such as LiF, NaCl, CsF, RbCl, RbI, CuI, or KI, a lanthanide metal such as Yb, and a co-deposited material of the metal halide and the lanthanide metal. For example, the electron transport region ETR may include KI:Yb, RbI:Yb, LiF:Yb, etc. as a co-deposited material. In some embodiments, the electron transport region ETR may be formed using a metal oxide such as Li₂O or BaO, or 8-hydroxyl-lithium quinolate (Liq), etc., but the embodiment of the present disclosure is not limited thereto. The electron transport region ETR may also be formed of a mixture material of an electron transport material and an insulating organometallic salt. The organometallic salt may be a material having an energy band gap of about 4 eV or more. For example, the organometallic salt may include, for example, a metal acetate, a metal benzoate, a metal acetoacetate, a metal acetylacetonate, or a metal stearate.

The electron transport region ETR may further include at least one of 2,9-dimethyl-4,7-diphenyl-1,10-phenanthroline (BCP), or 4,7-diphenyl-1,10-phenanthroline (Bphen) in addition to the above-described materials, but the embodiment of the present disclosure is not limited thereto.

The electron transport region ETR may include the above-described compounds of the hole transport region in at least one of the electron injection layer EIL, the electron transport layer ETL, or the hole blocking layer HBL.

When the electron transport region ETR includes the electron transport layer ETL, the electron transport layer ETL may have a thickness of about 100 Å to about 1,000 Å, for example, about 150 Å to about 500 Å. When the thickness of the electron transport layer ETL satisfies the aforementioned range, satisfactory (suitable) electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. When the thickness of the electron injection layer EIL satisfies the above-described range, satisfactory (suitable) electron injection characteristics may be obtained without a substantial increase in driving voltage.

The second electrode EL2 is provided on the electron transport region ETR. The second electrode EL2 may be a common electrode. The second electrode EL2 may be a cathode or an anode, but the embodiment of the present disclosure is not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, or one or more compounds or mixtures thereof (e.g., AgMg, AgYb, or MgAg). In some embodiments, the second electrode EL2 may have a multilayer structure including a reflective film or a transflective film formed of the above-described materials, and a transparent conductive film formed of ITO, IZO, ZnO, ITZO, etc. For example, the second electrode EL2 may include the above-described metal materials, combinations of at least two metal materials of the above-described metal materials, oxides of the above-described metal materials, and/or the like.

In some embodiments, the second electrode EL2 may be connected with an auxiliary electrode. When the second electrode EL2 is connected with the auxiliary electrode, the resistance of the second electrode EL2 may be decreased.

In some embodiments, a capping layer CPL may further be disposed on the second electrode EL2 of the light emitting element ED of an embodiment. The capping layer CPL may include a multilayer or a single layer. In an embodiment, the capping layer CPL may include the above-described amine compound of an embodiment.

In an embodiment, the capping layer CPL may be an organic layer or an inorganic layer. For example, when the capping layer CPL contains an inorganic material, the inorganic material may include an alkaline metal compound (for example, LiF), an alkaline earth metal compound (for example, MgF₂), SiON, SiNₓ, SiOy, etc.

For example, when the capping layer CPL contains an organic material, the organic material may include α-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol sol-9-yl)triphenylamine (TCTA), etc., or may include an epoxy resin, or an acrylate such as a methacrylate. However, the embodiment of the present disclosure is not limited thereto, and the capping layer CPL may include at least one selected from among Compounds P1 to P5:

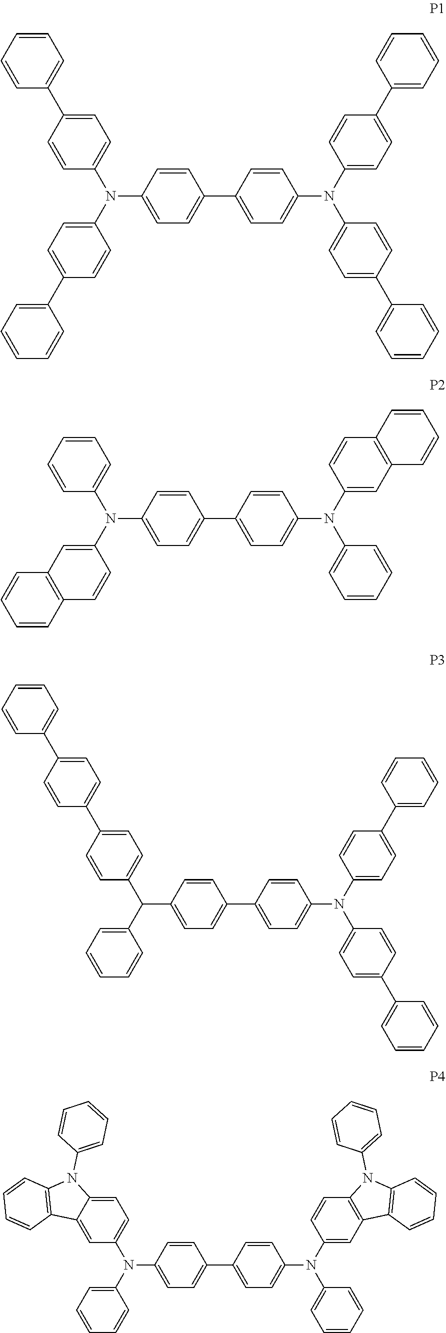 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(C(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

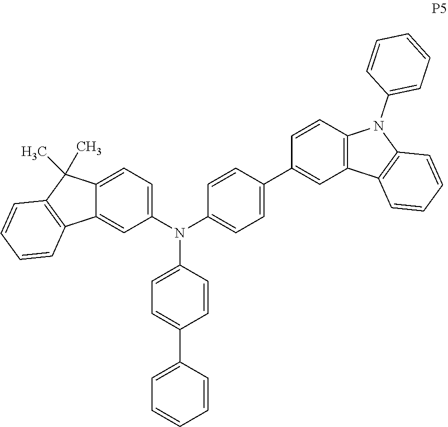 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL may be about 1.6 or more with respect to light in a wavelength range of about 550 nm to about 660 nm.

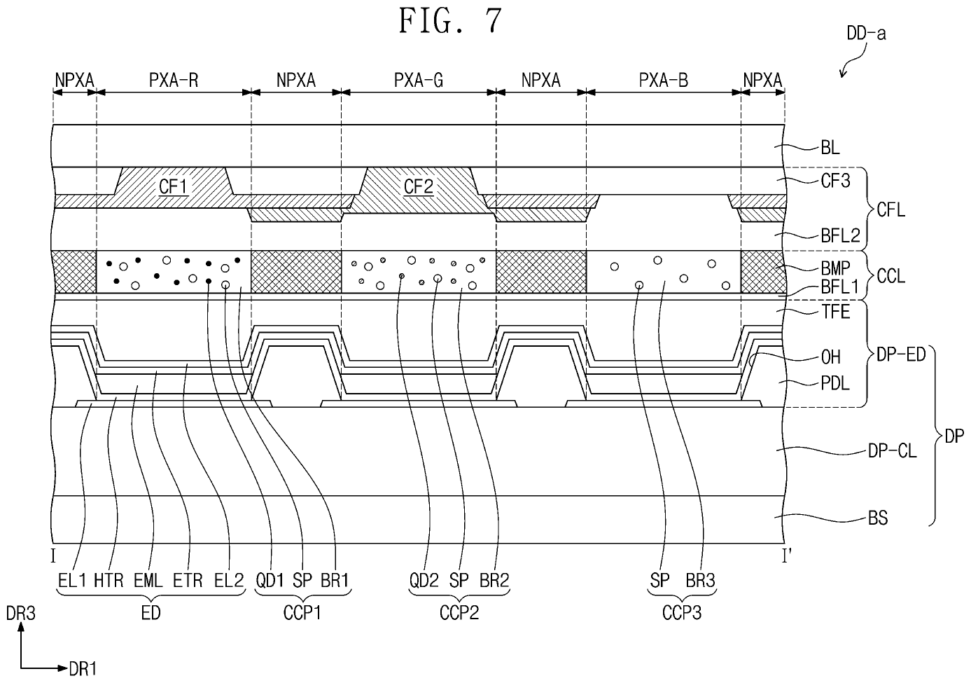

Each of FIGS. 7 to 10 is a cross-sectional view of a display device according to an embodiment of the present disclosure. Hereinafter, in describing the display devices of embodiments with reference to FIGS. 7 to 10, the duplicated features which have been described in FIGS. 1 to 6 may not be described again, but their differences will be primarily described.

Referring to FIG. 7, the display device DD-a according to an embodiment may include a display panel DP including a display element layer DP-ED, a light control layer CCL on the display panel DP, and a color filter layer CFL.

In an embodiment illustrated in FIG. 7, the display panel DP may include a base layer BS, a circuit layer DP-CL provided on the base layer BS, and the display element layer DP-ED, and the display element layer DP-ED may include a light emitting element ED.

The light emitting element ED may include a first electrode EL1, a hole transport region HTR on the first electrode EL1, an emission layer EML on the hole transport region HTR, an electron transport region ETR on the emission layer EML, and a second electrode EL2 on the electron transport region ETR. In some embodiments, the structures of the light emitting elements of FIGS. 3 to 6 as described above may be equally applied to the structure of the light emitting element ED illustrated in FIG. 7.

The hole transport region HTR of the light emitting element ED included in the display device DD-a according to an embodiment may include the above-described amine compound of an embodiment.

Referring to FIG. 7, the emission layer EML may be in an opening OH defined in a pixel defining film PDL. For example, the emission layer EML which is divided by the pixel defining film PDL and provided corresponding to each light emitting regions PXA-R, PXA-G, and PXA-B may emit light in substantially the same wavelength range. In the display device DD of an embodiment, the emission layer EML may emit blue light. In some embodiments, the emission layer EML may be provided as a common layer in the entire light emitting regions PXA-R, PXA-G, and PXA-B.

The light control layer CCL may be on the display panel DP. The light control layer CCL may include a light conversion body. The light conversion body may be a quantum dot, a phosphor, and/or the like. The light conversion body may emit provided light by converting the wavelength thereof. For example, the light control layer CCL may be a layer containing the quantum dot or a layer containing the phosphor.

The light control layer CCL may include a plurality of light control parts CCP1, CCP2 and CCP3. The light control parts CCP1, CCP2, and CCP3 may be spaced apart from each other.

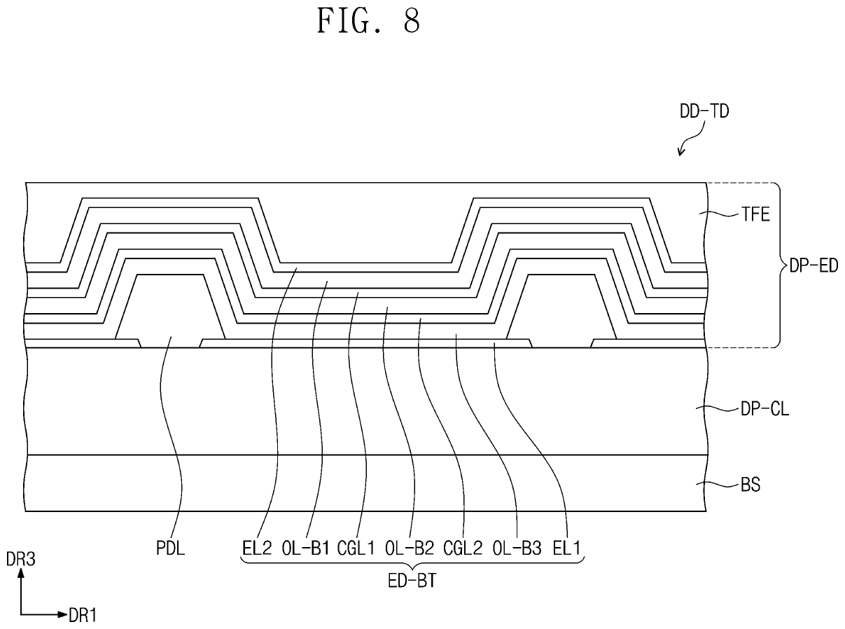

Referring to FIG. 7, divided patterns BMP may be between the light control parts CCP1, CCP2 and CCP3 which are spaced apart from each other, but the embodiment of the present disclosure is not limited thereto. FIG. 8 illustrates that the divided patterns BMP do not overlap the light control parts CCP1, CCP2 and CCP3, but at least a portion of the edges of the light control parts CCP1, CCP2 and CCP3 may overlap the divided patterns BMP.

The light control layer CCL may include a first light control part CCP1 containing a first quantum dot QD1 which converts first color light provided from the light emitting element ED into second color light, a second light control part CCP2 containing a second quantum dot QD2 which converts the first color light into third color light, and a third light control part CCP3 which transmits the first color light.

In an embodiment, the first light control part CCP1 may provide red light that is the second color light, and the second light control part CCP2 may provide green light that is the third color light. The third light control part CCP3 may provide blue light by transmitting the blue light that is the first color light provided from the light emitting element ED. For example, the first quantum dot QD1 may be a red quantum dot, and the second quantum dot QD2 may be a green quantum dot. The same description used above may be applied with respect to the quantum dots QD1 and QD2.

In some embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but include the scatterer SP.

The scatterer SP may be inorganic particles. For example, the scatterer SP may include at least one of TiO₂, ZnO, Al₂O₃, SiO₂, or hollow sphere silica. The scatterer SP may include any one selected from among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 each may include base resins BR1, BR2, and BR3 in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3. The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may be acrylic-based resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 may be the same as or different from each other.

The light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may be on the light control parts CCP1, CCP2, and CCP3 to block or reduce the light control parts CCP1, CCP2 and CCP3 from being exposed to moisture/oxygen. In some embodiments, the barrier layer BFL1 may cover the light control parts CCP1, CCP2, and CCP3. In some embodiments, a barrier layer BFL2 may be provided between the light control parts CCP1, CCP2, and CCP3 and filters CF-B, CF-G, and CF-R.

The barrier layers BFL1 and BFL2 may include at least one inorganic layer. For example, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, etc. In some embodiments, the barrier layers BFL1 and BFL2 may further include an organic film. The barrier layers BFL1 and BFL2 may be formed of a single layer or a plurality of layers.

In the display device DD of an embodiment, the color filter layer CFL may be on the light control layer CCL. For example, the color filter layer CFL may be directly on the light control layer CCL. In this embodiment, the barrier layer BFL2 may not be provided.

The color filter layer CFL may include a light shielding unit BM and filters CF-B, CF-G, and CF-R. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and a pigment or dye. The first filter CF1 may include a red pigment or dye, the second filter CF2 may include a green pigment or dye, and the third filter CF3 may include a blue pigment or dye. However, the embodiment of the present disclosure is not limited thereto, and the third filter CF3 may not include a pigment or dye. The third filter CF3 may include a polymeric photosensitive resin and may not include (e.g., may exclude) a pigment or dye. The third filter CF3 may be transparent. The third filter CF3 may be formed of a transparent photosensitive resin.

Furthermore, in an embodiment, the first filter CF1 and the second filter CF2 may be a yellow filter. The first filter CF1 and the second filter CF2 may not be separated but be provided as one filter.

The light shielding part BM may be a black matrix. The light shielding part BM may include an organic light shielding material or an inorganic light shielding material containing a black pigment or dye. The light shielding part BM may prevent light leakage, and may separate boundaries between the adjacent filters CF1, CF2, and CF3. In some embodiments, the light shielding part BM may be formed of a blue filter.

The first to third filters CF1, CF2, and CF3 may be disposed corresponding to the red light emitting region PXA-R, the green light emitting region PXA-G, and the blue light emitting region PXA-B, respectively.

A base substrate BL may be on the color filter layer CFL. The base substrate BL may be a member which provides a base surface in which the color filter layer CFL, the light control layer CCL, and/or the like are disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In some embodiments, the base substrate BL may not be provided.

FIG. 8 is a cross-sectional view illustrating a portion of a display device according to an embodiment of the present disclosure. FIG. 8 illustrates a cross-sectional view of a part corresponding to the display panel DP of FIG. 7. In the display device DD-TD of an embodiment, the light emitting element ED-BT may include a plurality of light emitting structures OL-B1, OL-B2, and OL-B3. The light emitting element ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. The light emitting structures OL-B1, OL-B2, and OL-B3 each may include an emission layer EML (FIG. 7) and a hole transport region HTR and an electron transport region ETR disposed with the emission layer EML (FIG. 7) located therebetween.

For example, the light emitting element ED-BT included in the display device DD-TD of an embodiment may be a light emitting element having a tandem structure and including a plurality of emission layers.

In an embodiment illustrated in FIG. 8, all light beams respectively emitted from the light emitting structures OL-B1, OL-B2, and OL-B3 may be blue light. However, the embodiment of the present disclosure is not limited thereto, and the light beams respectively emitted from the light emitting structures OL-B1, OL-B2, and OL-B3 may have wavelength ranges different from each other. For example, the light emitting element ED-BT including the plurality of light emitting structures OL-B1, OL-B2, and OL-B3 which emit light beams having wavelength ranges different from each other may emit white light.

Charge generation layers CGL1 and CGL2 may be respectively disposed between two of the neighboring light emitting structures OL-B1, OL-B2, and OL-B3. The charge generation layers CGL1 and CGL2 may include a p-type or kind charge generation layer and/or an n-type or kind charge generation layer.

At least one of the light emitting structures OL-B1, OL-B2, or OL-B3 included in the display device DD-TD of an embodiment may contain the above-described amine compound of an embodiment.

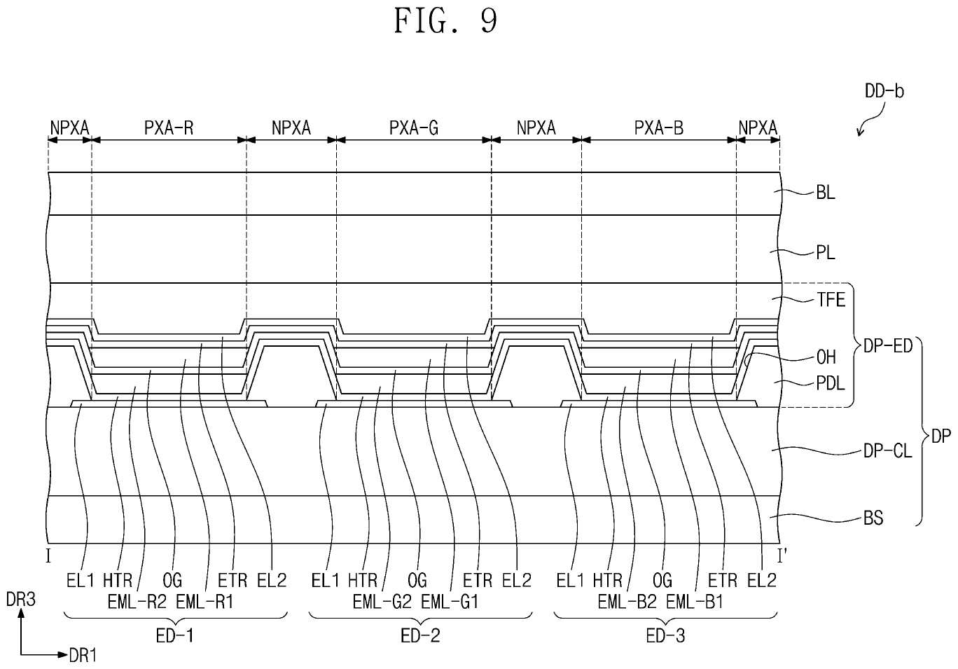

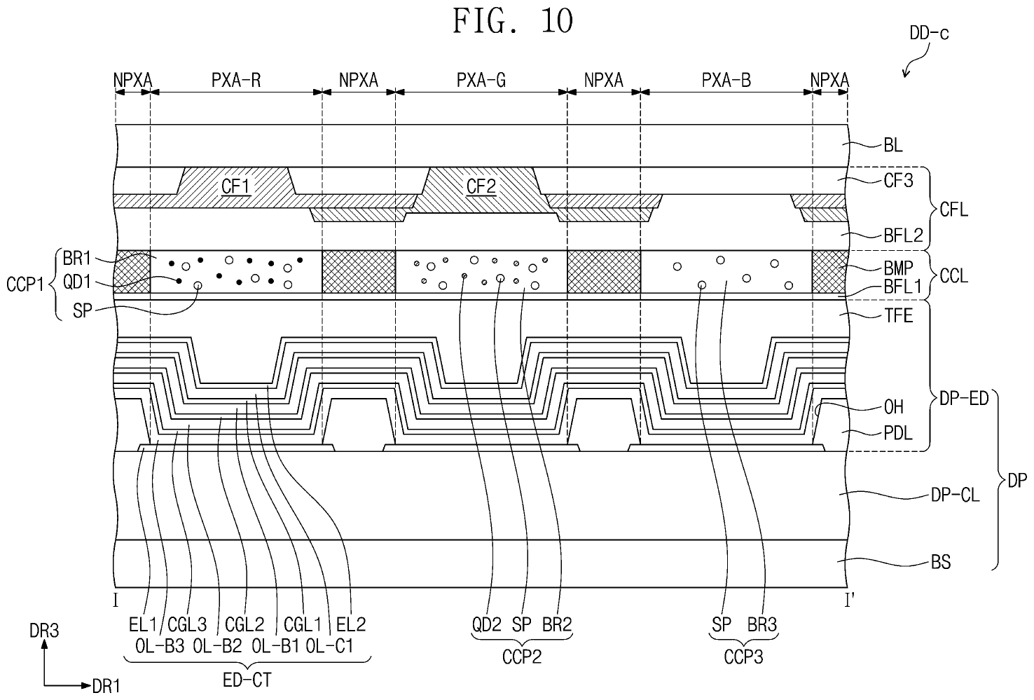

Referring to FIG. 9, the display device DD-b according to an embodiment may include light emitting elements ED-1, ED-2, and ED-3 in which two emission layers are stacked. Compared with the display device DD of an embodiment illustrated in FIG. 2, an embodiment illustrated in FIG. 9 has a difference in that the first to third light emitting elements ED-1, ED-2, and ED-3 each include two emission layers stacked in the thickness direction. In each of the first to third light emitting elements ED-1, ED-2, and ED-3, the two emission layers may emit light in substantially the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. An emission auxiliary part OG may be between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. For example, the emission auxiliary part OG may include an electron transport region, a charge generation layer, and a hole transport region that are sequentially stacked. The emission auxiliary part OG may be provided as a common layer in the whole of the first to third light emitting elements ED-1, ED-2, and ED-3. However, the embodiment of the present disclosure is not limited thereto, and the emission auxiliary part OG may be provided by being patterned within the openings OH defined in the pixel defining film PDL.

The first red emission layer EML-R1, the first green emission layer EML-G1, and the first blue emission layer EML-B1 may be between the hole transport region HTR and the emission auxiliary part OG. The second red emission layer EML-R2, the second green emission layer EML-G2, and the second blue emission layer EML-B2 may be between the emission auxiliary part OG and the electron transport region ETR.

For example, the first light emitting element ED-1 may include the first electrode EL1, the hole transport region HTR, the second red emission layer EML-R2, the emission auxiliary part OG, the first red emission layer EML-R1, the electron transport region ETR, and the second electrode EL2 that are sequentially stacked. The second light emitting element ED-2 may include the first electrode EL1, the hole transport region HTR, the second green emission layer EML-G2, the emission auxiliary part OG, the first green emission layer EML-G1, the electron transport region ETR, and the second electrode EL2 that are sequentially stacked. The third light emitting element ED-3 may include the first electrode EL1, the hole transport region HTR, the second blue emission layer EML-B2, the emission auxiliary part OG, the first blue emission layer EML-B1, the electron transport region ETR, and the second electrode EL2 that are sequentially stacked.

In some embodiments, an optical auxiliary layer PL may be on the display element layer DP-ED. The optical auxiliary layer PL may include a polarizing layer. The optical auxiliary layer PL may be on the display panel DP and control reflected light in the display panel DP due to external light. In an embodiment, the optical auxiliary layer PL in the display device may not be provided.

Unlike FIGS. 8 and 9, FIG. 10 illustrates that a display device DD-c includes four light emitting structures OL-B1, OL-B2, OL-B3, and OL-C1. A light emitting element ED-CT may include a first electrode EL1 and a second electrode EL2 which face each other, and first to fourth light emitting structures OL-B1, OL-B2, OL-B3, and OL-C1 that are sequentially stacked in the thickness direction between the first electrode EL1 and the second electrode EL2. Charge generation layers CGL1, CGL2, and CGL3 may be between the first to fourth light emitting structures OL-B1, OL-B2, OL-B3, and OL-C1. Among the four light emitting structures, the first to third light emitting structures OL-B1, OL-B2, and OL-B3 may emit blue light, and the fourth light emitting structure OL-C1 may emit green light. However, the embodiment of the present disclosure is not limited thereto, and the first to fourth light emitting structures OL-B1, OL-B2, OL-B3, and OL-C1 may emit light beams in different wavelength regions.

The charge generation layers CGL1, CGL2, and CGL3 between adjacent light emitting structures OL-B1, OL-B2, OL-B3, and OL-C1 may include a p-type or kind charge generation layer and/or an n-type or kind charge generation layer.

At least one among the light emitting structures OL-B1, OL-B2, OL-B3, and OL-C1 included in the display device DD-c of an embodiment may include the above-described amine compound of an embodiment.

The light emitting element ED according to an embodiment of the present disclosure may include the above-described amine compound of an embodiment in at least one functional layer between the first electrode EL1 and the second electrode EL2, thereby exhibiting a low driving voltage characteristic. The light emitting element ED according to an embodiment may include the above-described amine compound of an embodiment in at least one of the hole transport region HTR, the emission layer EML, or the electron transport region ETR between the first electrode EL1 and the second electrode EL2, or in a capping layer CPL.

For example, the amine compound according to an embodiment may be included in the hole transport region HTR of the light emitting element ED of an embodiment, and the light emitting element of an embodiment may exhibit a low driving voltage characteristic.

The above-described amine compound of an embodiment includes a 9-phenyl-9H-carbazole group directly linked to the nitrogen atom, a substituted or unsubstituted naphthyl group linked via a linker, and an aryl group or heteroaryl group directly linked to the nitrogen atom, and thus may reduce the driving voltage.

Hereinafter, with reference to Examples and Comparative Examples, an amine compound according to an embodiment of the present disclosure and a light emitting element of an embodiment of the present disclosure will be described in more detail. In addition, Examples described below are merely examples to assist the understanding of the present disclosure, and the scope of the present disclosure is not limited thereto.

### EXAMPLES

### 1. Synthesis of Amine Compound

First, a synthetic method of an amine compound according to the present embodiment will be described in more detail by illustrating the synthetic methods of Compounds A9, A12, F12, H9, H12, N12, O22, O23, O32, P12, P23, and P37. Also, in the following descriptions, the synthetic method of the amine compound is provided as an example, but the synthetic method according to an embodiment of the present disclosure is not limited to Examples below.

Compounds A9, A12, F12, H9, H12, N12, O22, O23, O32, P12, P23, and P37 that are Example Compounds were synthesized by using Compounds X1 to X20 as follows:

### (1) Synthesis of Compound X3

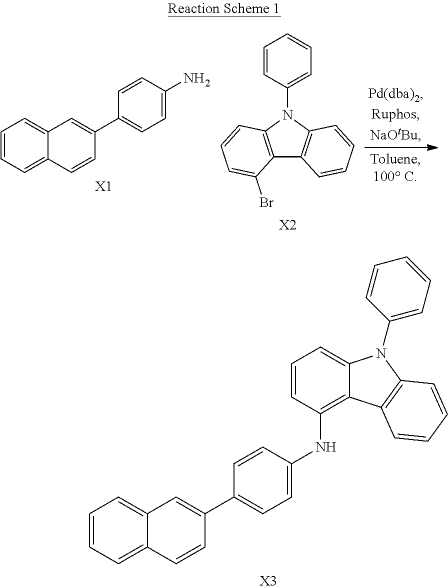

Toluene (200 mL) was added to Compound X1 (2.2 g, 10 mmol), Compound X2 (3.2 g, 10 mmol), NaOtBu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound X3 (4.1 g, 9.0 mmol, yield 90%, MS 460.19).

### (2) Synthesis of Compound X5

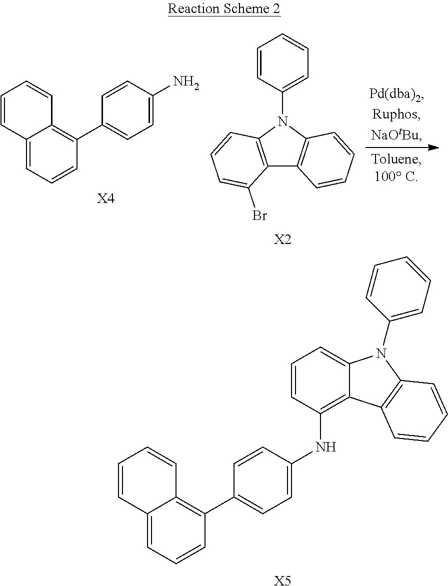

Toluene (200 mL) was added to Compound X4 (2.2 g, 10 mmol), Compound X2 (3.2 g, 10 mmol), NaOtBu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound X5 (3.7 g, 8.0 mmol, yield 80%, MS 460.19).

### (3) Synthesis of Compound X7

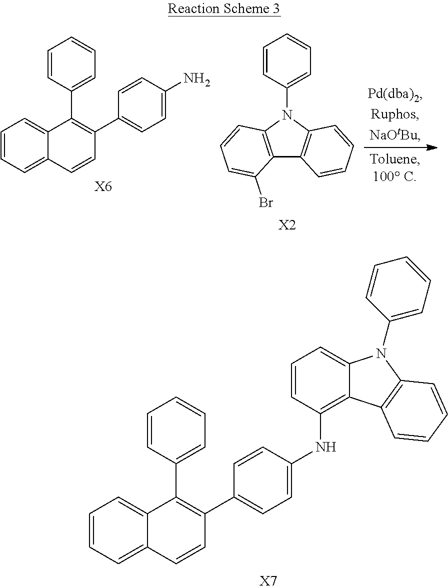

Toluene (200 mL) was added to Compound X6 (3.0 g, 10 mmol), Compound X2 (3.2 g, 10 mmol), NaO^{t}Bu (0.96 g, 10 mmol), RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound X7 (3.5 g, 8.0 mmol, yield 65%, MS 536.23).

### (4) Synthesis of Compound X9

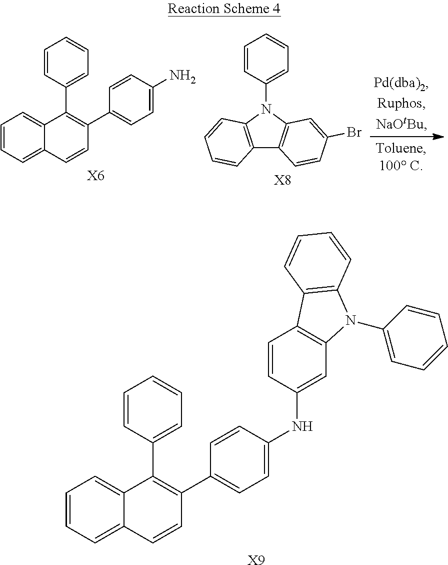

Toluene (200 mL) was added to Compound X6 (3.0 g, 10 mmol), Compound X8 (3.2 g, 10 mmol), NaOtBu (0.96 g, 10 mmol), RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound X9 (3.3 g, 6.1 mmol, yield 61%, MS 536.23).

### (5) Synthesis of Compound X11

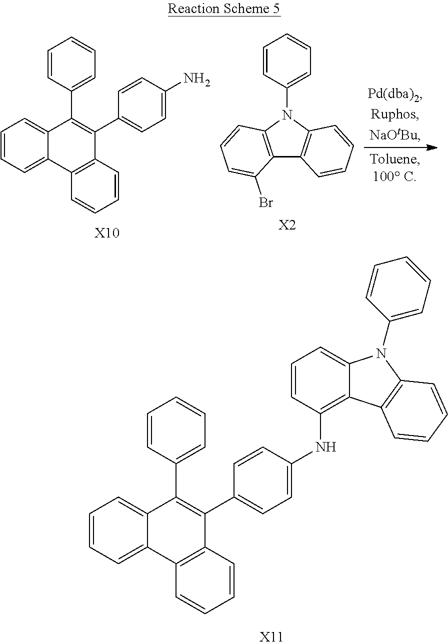

Toluene (200 mL) was added to Compound X10 (3.5 g, 10 mmol), Compound X2 (3.2 g, 10 mmol), NaOtBu (0.96 g, 10 mmol), RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound X11 (3.4 g, 5.8 mmol, yield 58%, MS 586.24).

### (6) Synthesis of Compound X13

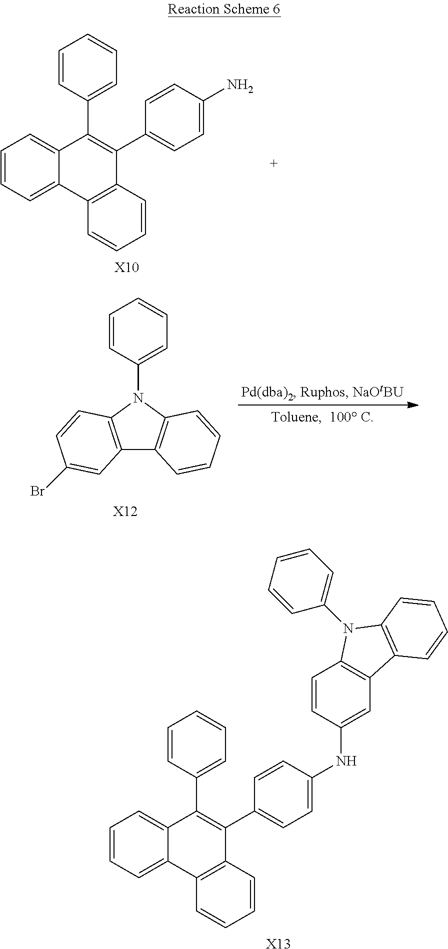

Toluene (200 mL) was added to Compound X10 (3.5 g, 10 mmol), Compound X12 (3.2 g, 10 mmol), NaOtBu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound X13 (3.7 g, 6.3 mmol, yield 63%, MS 586.24).

### (7) Synthesis of Compound A₉

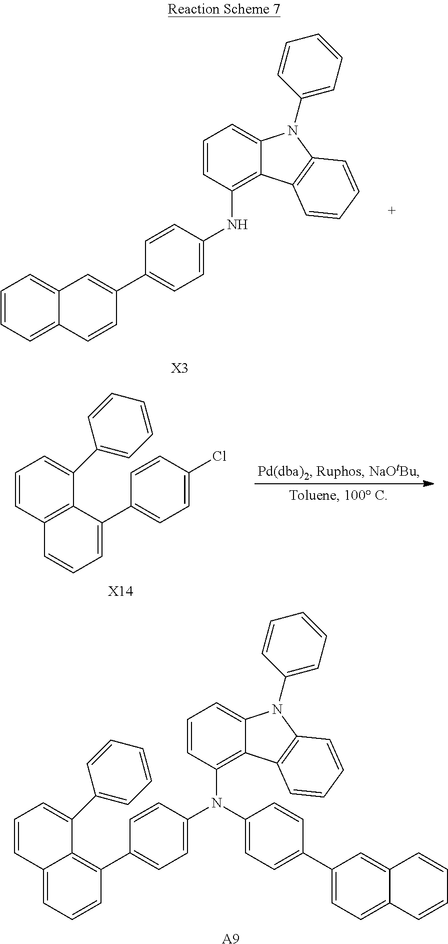

Toluene (200 mL) was added to Compound X3 (4.6 g, 10 mmol), Compound X14 (3.1 g, 10 mmol), NaOtBu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound A9 (5.5 g, 7.5 mmol, yield 75%, MS 738.30).

### (8) Synthesis of Compound A12

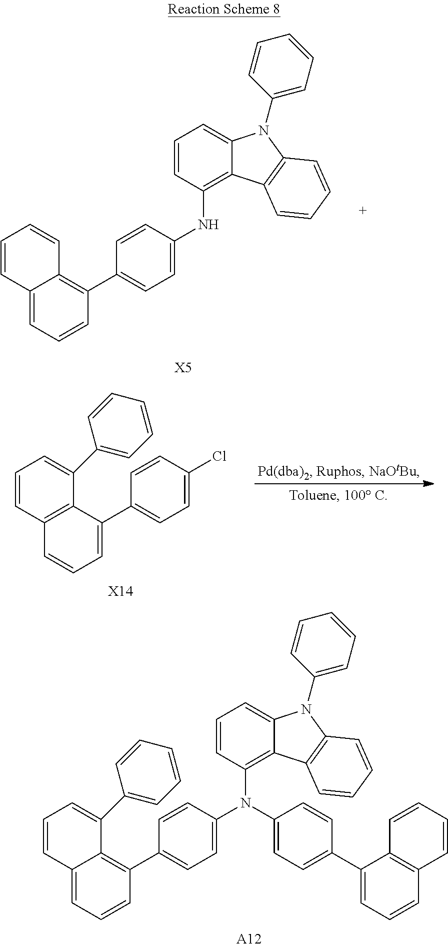

Toluene (200 mL) was added to Compound X5 (4.6 g, 10 mmol), Compound X14 (3.1 g, 10 mmol), NaOtBu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound A12 (5.2 g, 7.1 mmol, yield 71%, MS 738.30).

### (9) Synthesis of Compound F12

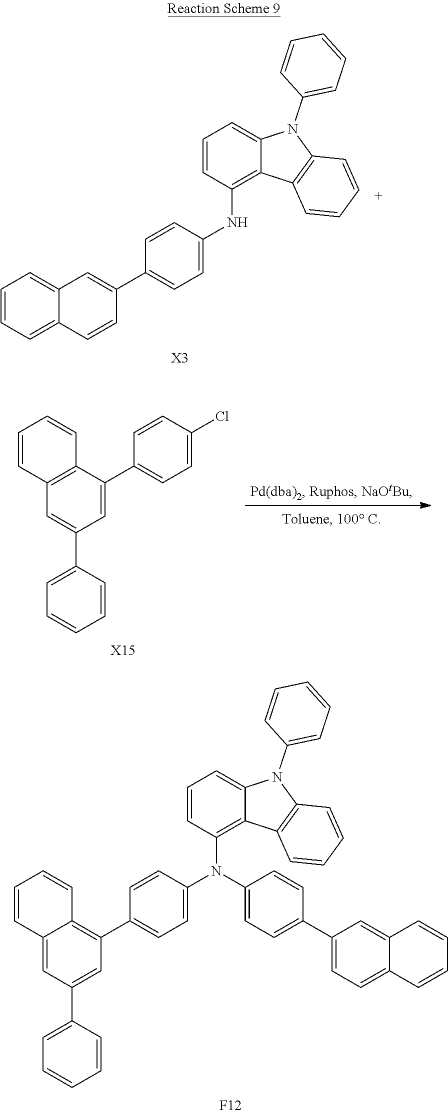

Toluene (200 mL) was added to Compound X3 (4.6 g, 10 mmol), Compound X15 (3.1 g, 10 mmol), NaOtBu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound F12 (5.2 g, 7.0 mmol, yield 70%, MS 738.30).

### (10) Synthesis of Compound O22

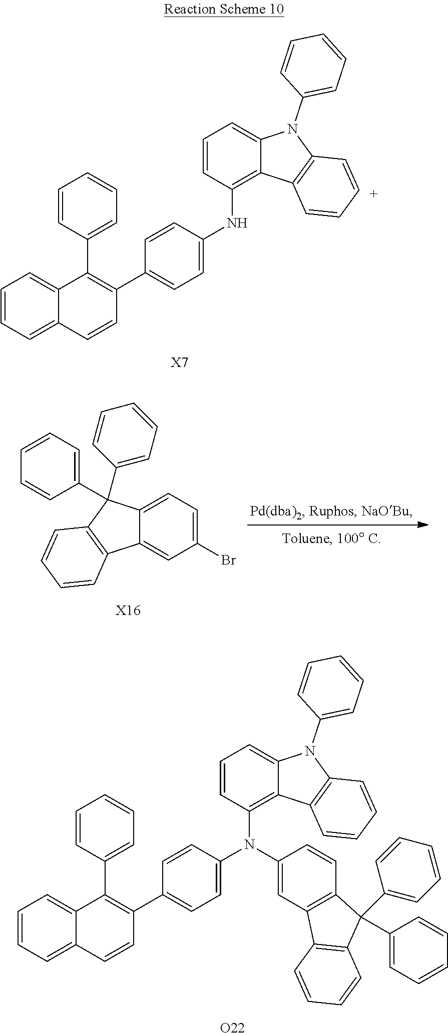

Toluene (200 mL) was added to Compound X7 (5.4 g, 10 mmol), Compound X16 (4.0 g, 10 mmol), NaOtBu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound O22 (5.5 g, 6.5 mmol, yield 65%, MS 852.35).

### (11) Synthesis of Compound O23

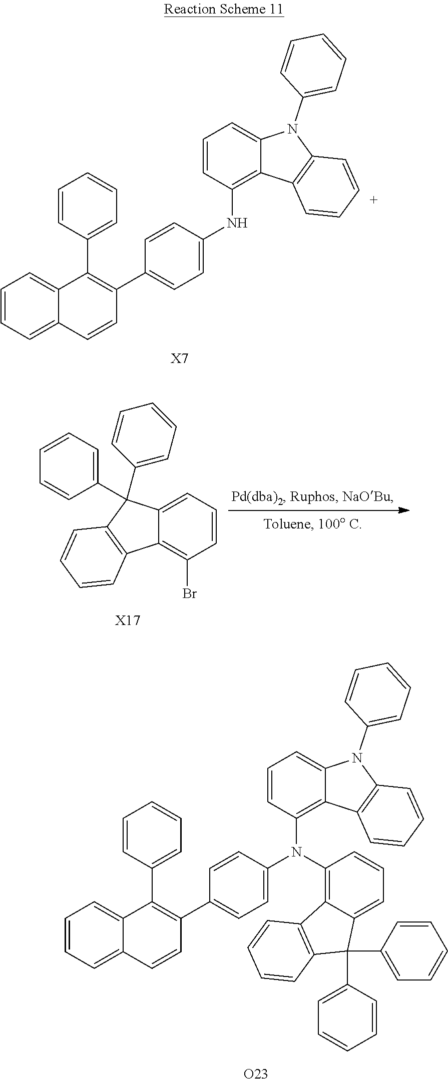

Toluene (200 mL) was added to Compound X7 (5.4 g, 10 mmol), Compound X17 (4.0 g, 10 mmol), Na^{t}Bu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound O23 (6.0 g, 7.1 mmol, yield 71%, MS 852.35).

### (12) Synthesis of Compound O32

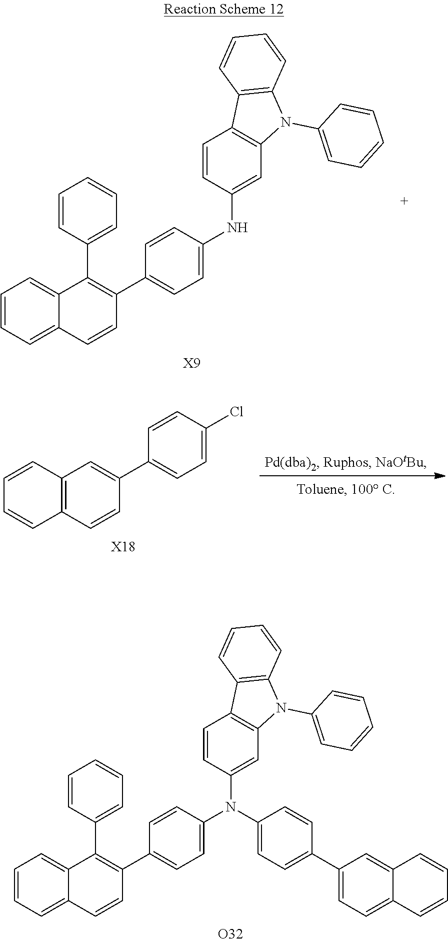

Toluene (200 mL) was added to Compound X9 (5.4 g, 10 mmol), Compound X18 (2.4 g, 10 mmol), NaO^{t}Bu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound O32 (6.0 g, 8.1 mmol, yield 81%, MS 738.30).

### (13) Synthesis of Compound H9

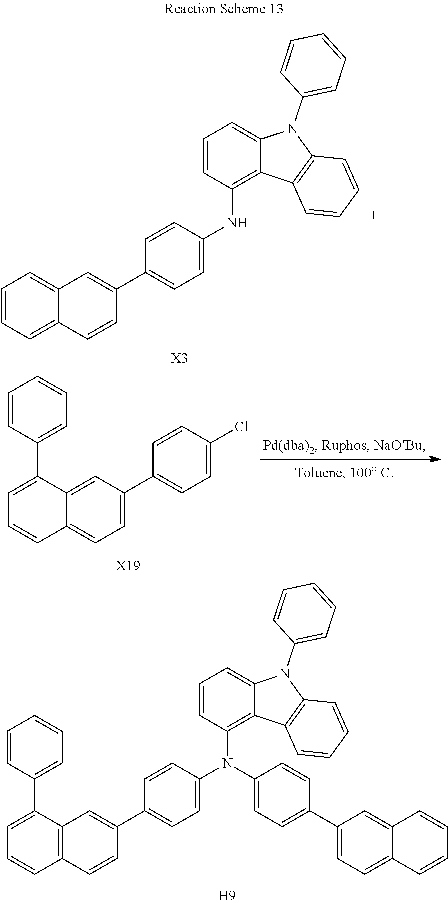

Toluene (200 mL) was added to Compound X3 (4.6 g, 10 mmol), Compound X19 (3.1 g, 10 mmol), NaO^{t}Bu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound H9 (5.5 g, 7.5 mmol, yield 75%, MS 738.30).

### (14) Synthesis of Compound H12

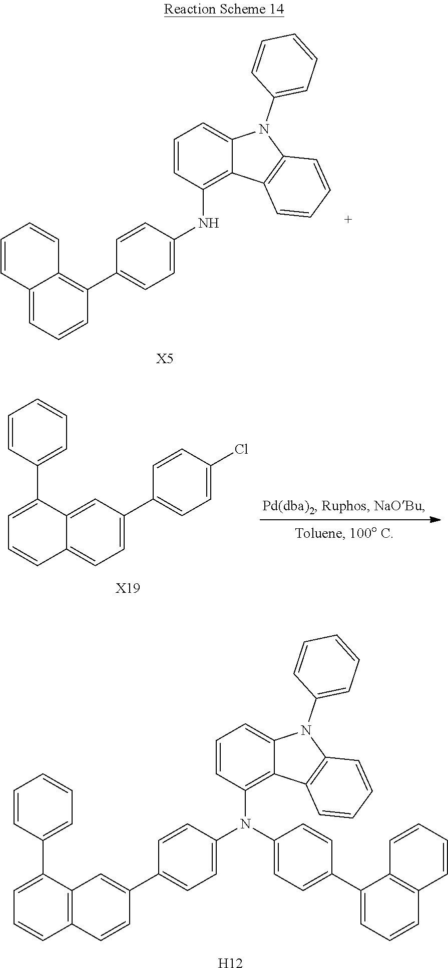

Toluene (200 mL) was added to Compound X5 (4.6 g, 10 mmol), Compound X19 (3.1 g, 10 mmol), NaOtBu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound H12 (5.4 g, 7.3 mmol, yield 73%, MS 738.30).

### (15) Synthesis of Compound N12

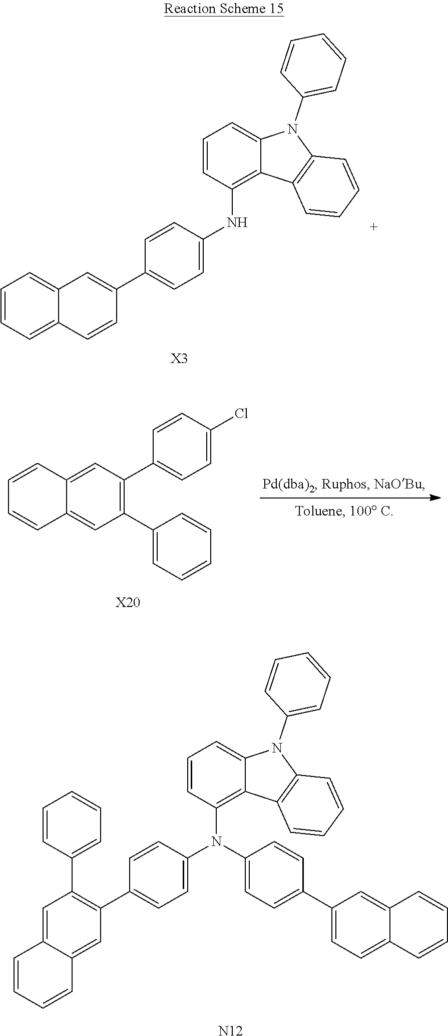

Toluene (200 mL) was added to Compound X3 (4.6 g, 10 mmol), Compound X20 (3.1 g, 10 mmol), NaO^{t}Bu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound N12 (5.1 g, 6.9 mmol, yield 69%, MS 738.30).

### (16) Synthesis of Compound P12

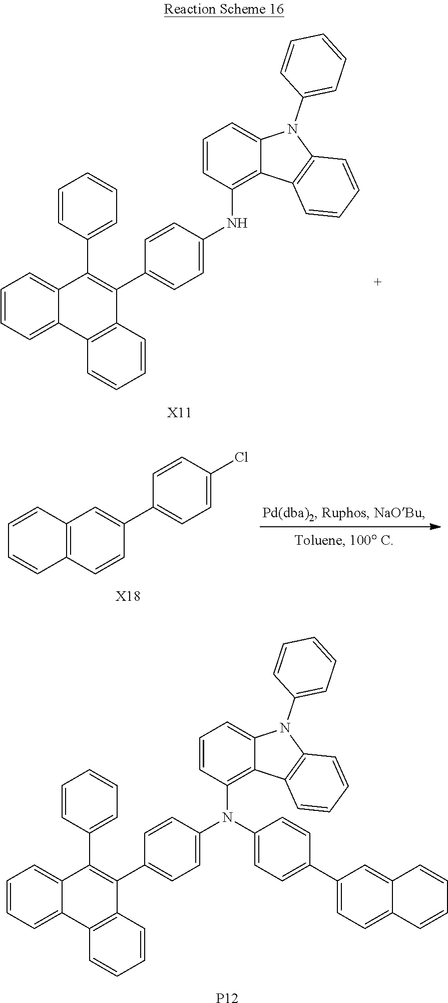

Toluene (200 mL) was added to Compound X11 (5.9 g, 10 mmol), Compound X18 (2.4 g, 10 mmol), NaO^{t}Bu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound P12 (5.8 g, 7.4 mmol, yield 74%, MS 788.30).

### (17) Synthesis of Compound P23

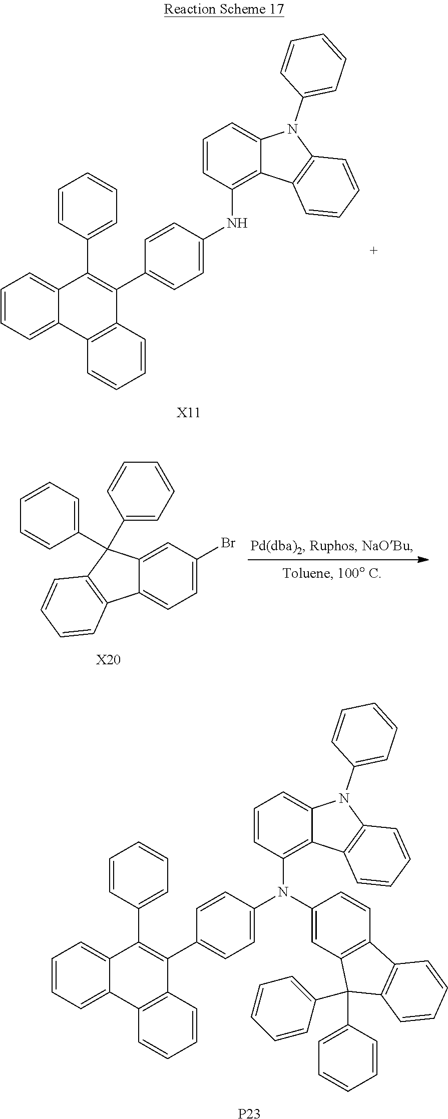

Toluene (200 mL) was added to Compound X11 (5.9 g, 10 mmol), Compound X20 (4.0 g, 10 mmol), NaO^{t}Bu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound P23 (7.6 g, 8.4 mmol, yield 84%, MS 902.37).

### (18) Synthesis of Compound P37

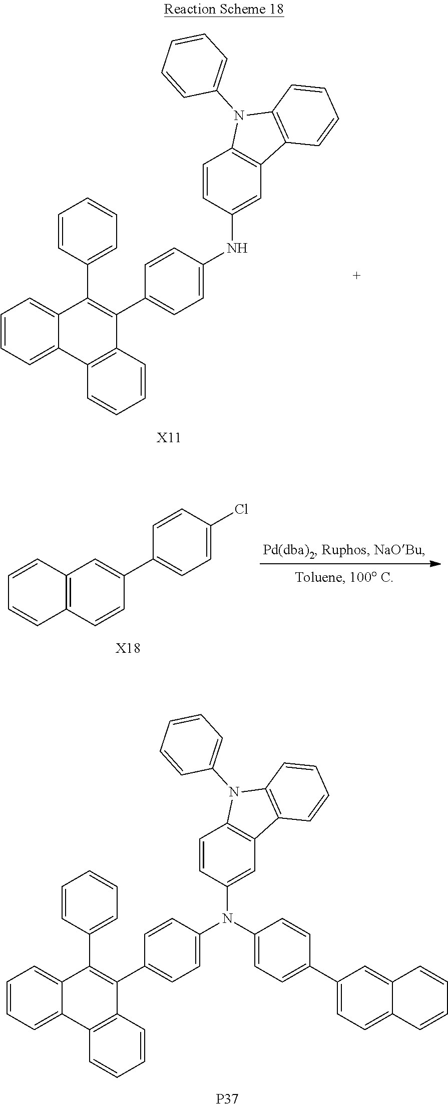

Toluene (200 mL) was added to Compound X11 (5.9 g, 10 mmol), Compound X18 (2.4 g, 10 mmol), NaO^{t}Bu (0.96 g, 10 mmol), and RuPhos (0.46 g, 1 mmol), and the resulting mixture was degassed. In an argon atmosphere, bis(dibenzylideneacetone)palladium (0.29 g, 0.5 mmol) was added thereto, and the resultant mixture was heated and stirred at about 100° C. for about 6 hours. The reaction solution was allowed to cool to room temperature, extracted with toluene, washed with H₂O and brine, and dried over Na₂SO₄. The obtained solution was concentrated and purified by column chromatography to obtain Compound P37 (5.3 g, 6.7 mmol, yield 67%, MS 788.30).

### 2. Manufacture and Evaluation of Light Emitting Element

Evaluation of the light emitting elements including compounds of Examples and Comparative Examples in a hole transport layer was performed as follows. The method for manufacturing the light emitting element for the evaluation of the element is described below.

### (1) Manufacture of Light Emitting Elements

A glass substrate on which a 150 nm-thick ITO had been patterned was ultrasonically washed by using isopropyl alcohol and pure water for about 5 minutes each. After ultrasonically washed, the glass substrate was irradiated with UV rays for about 30 minutes and treated with ozone. 2-TNATA was deposited to form a 60 nm-thick hole injection layer.

Then, Example Compound or Comparative Example Compound was deposited to form a 30 nm-thick hole transport layer. Thereafter, ADN and TBP were co-deposited in a weight ratio of about 97:3 to form a 25 nm-thick emission layer. Then, Alq₃ was deposited to form a 25 nm-thick electron transport layer, and LiF was deposited to form a 1 nm-thick electron injection layer.

Next, Al was deposited to form a 100 nm-thick second electrode.

In the Examples, the hole injection layer, the hole transport layer, the emission layer, the electron transport layer, the electron injection layer, and the second electrode were formed by using a vacuum deposition apparatus.

Example Compounds and Comparative Example Compounds used to manufacture the light emitting elements are as follows:

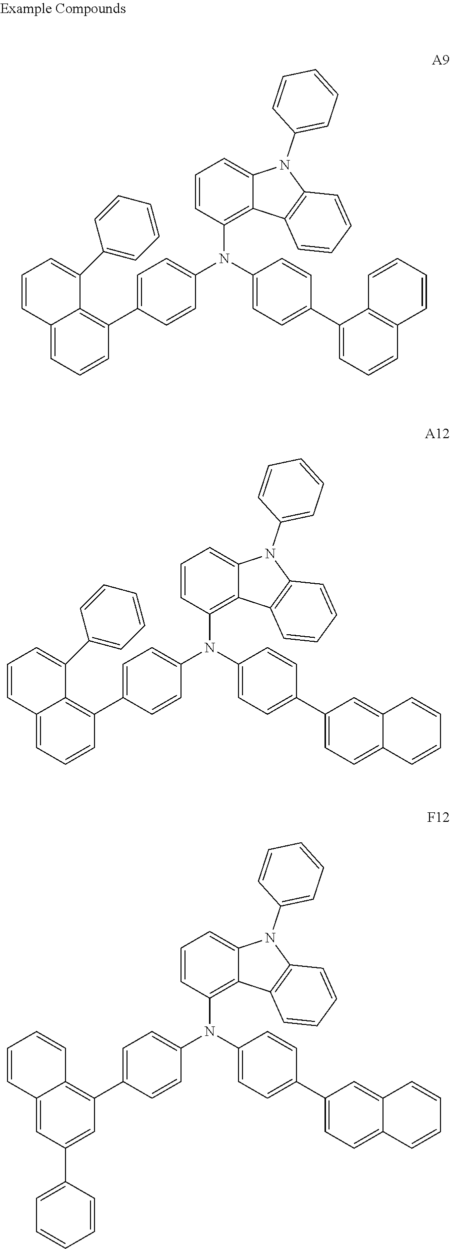 [SMILES: c1ccc(-c2cccc3cccc(-c4ccc(N(c5ccc(-c6cccc7ccccc67)cc5)c5cccc6c5c5ccccc5n6-c5ccccc5)cc4)c23)cc1 ; c1ccc(-c2cccc3cccc(-c4ccc(N(c5ccc(-c6ccc7ccccc7c6)cc5)c5cccc6c5c5ccccc5n6-c5ccccc5)cc4)c23)cc1 ; c1ccc(-c2cc(-c3ccc(N(c4ccc(-c5ccc6ccccc6c5)cc4)c4cccc5c4c4ccccc4n5-c4ccccc4)cc3)c3ccccc3c2)cc1]

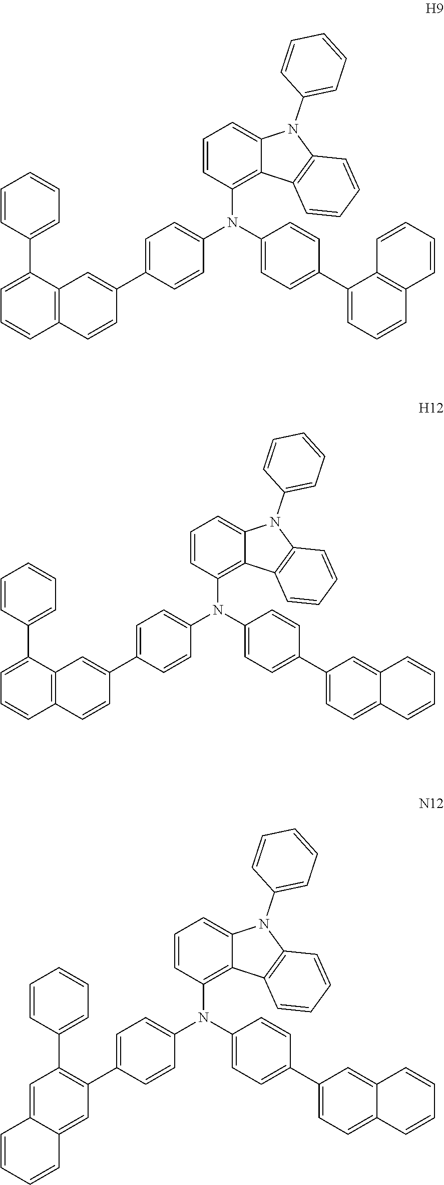 [SMILES: c1ccc(-c2cccc3ccc(-c4ccc(N(c5ccc(-c6cccc7ccccc67)cc5)c5cccc6c5c5ccccc5n6-c5ccccc5)cc4)cc23)cc1 ; c1ccc(-c2cccc3ccc(-c4ccc(N(c5ccc(-c6ccc7ccccc7c6)cc5)c5cccc6c5c5ccccc5n6-c5ccccc5)cc4)cc23)cc1 ; c1ccc(-c2cc3ccccc3cc2-c2ccc(N(c3ccc(-c4ccc5ccccc5c4)cc3)c3cccc4c3c3ccccc3n4-c3ccccc3)cc2)cc1]

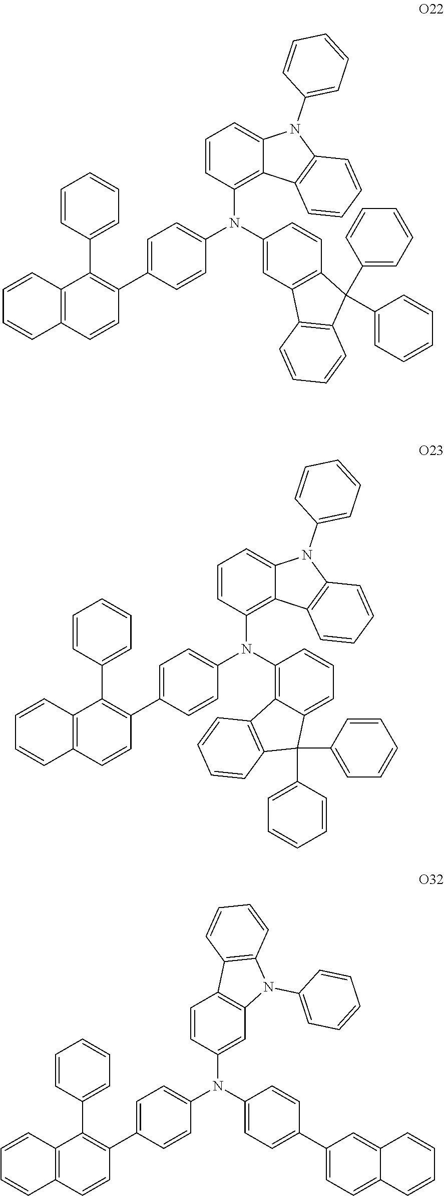 [SMILES: c1ccc(-c2c(-c3ccc(N(c4ccc5c(c4)-c4ccccc4C5(c4ccccc4)c4ccccc4)c4cccc5c4c4ccccc4n5-c4ccccc4)cc3)ccc3ccccc23)cc1 ; c1ccc(-c2c(-c3ccc(N(c4cccc5c4-c4ccccc4C5(c4ccccc4)c4ccccc4)c4cccc5c4c4ccccc4n5-c4ccccc4)cc3)ccc3ccccc23)cc1 ; c1ccc(-c2c(-c3ccc(N(c4ccc(-c5ccc6ccccc6c5)cc4)c4ccc5c6ccccc6n(-c6ccccc6)c5c4)cc3)ccc3ccccc23)cc1]

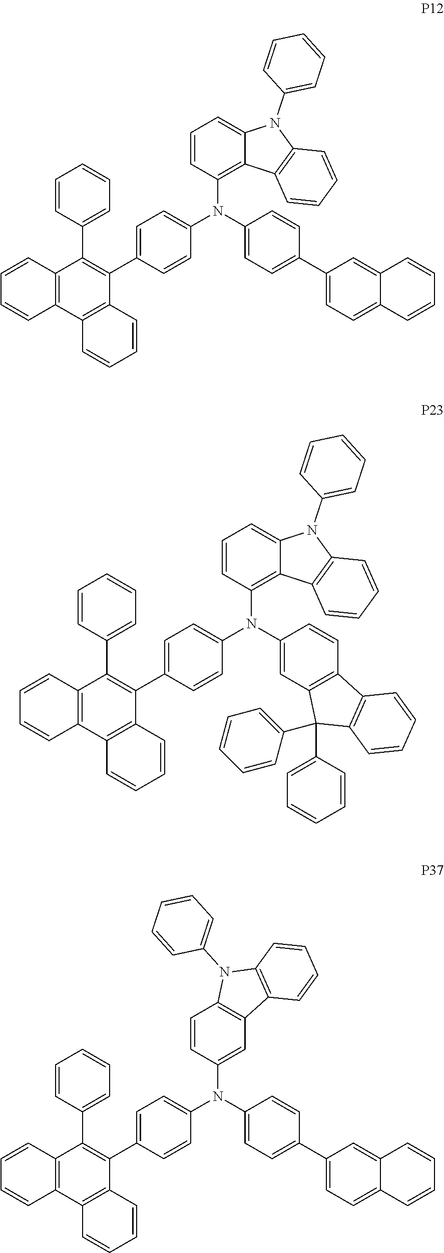 [SMILES: c1ccc(-c2c(-c3ccc(N(c4ccc(-c5ccc6ccccc6c5)cc4)c4cccc5c4c4ccccc4n5-c4ccccc4)cc3)c3ccccc3c3ccccc23)cc1 ; c1ccc(-c2c(-c3ccc(N(c4ccc5c(c4)C(c4ccccc4)(c4ccccc4)c4ccccc4-5)c4cccc5c4c4ccccc4n5-c4ccccc4)cc3)c3ccccc3c3ccccc23)cc1 ; c1ccc(-c2c(-c3ccc(N(c4ccc(-c5ccc6ccccc6c5)cc4)c4ccc5c(c4)c4ccccc4n5-c4ccccc4)cc3)c3ccccc3c3ccccc23)cc1]

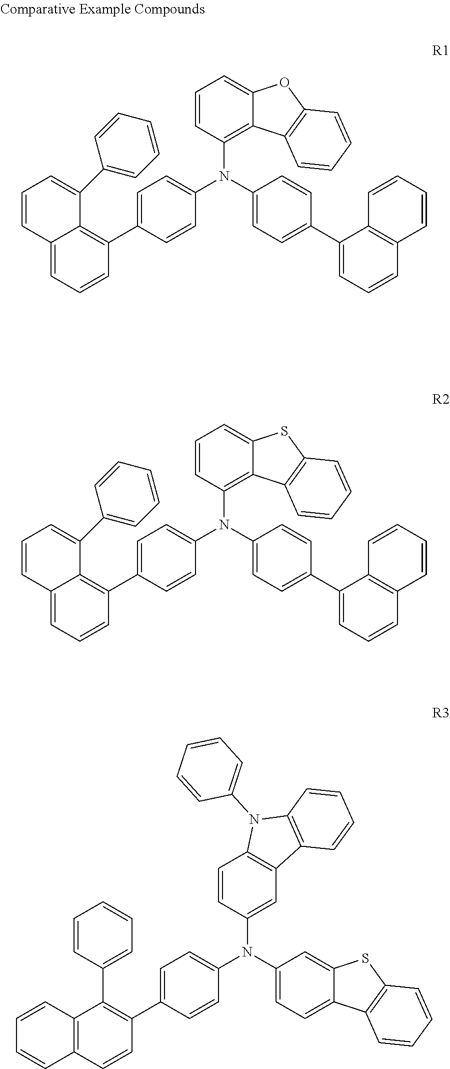 [SMILES: c1ccc(-c2cccc3cccc(-c4ccc(N(c5ccc(-c6cccc7ccccc67)cc5)c5cccc6oc7ccccc7c56)cc4)c23)cc1 ; c1ccc(-c2cccc3cccc(-c4ccc(N(c5ccc(-c6cccc7ccccc67)cc5)c5cccc6sc7ccccc7c56)cc4)c23)cc1 ; c1ccc(-c2c(-c3ccc(N(c4ccc5c(c4)sc4ccccc45)c4ccc5c(c4)c4ccccc4n5-c4ccccc4)cc3)ccc3ccccc23)cc1]

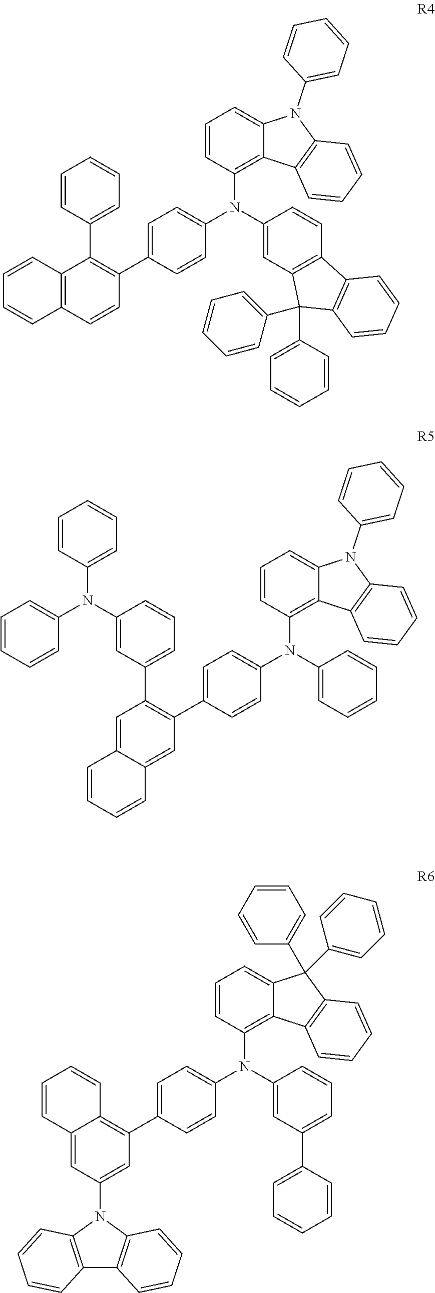 [SMILES: c1ccc(-c2c(-c3ccc(N(c4ccc5c(c4)C(c4ccccc4)(c4ccccc4)c4ccccc4-5)c4cccc5c4c4ccccc4n5-c4ccccc4)cc3)ccc3ccccc23)cc1 ; c1ccc(N(c2ccccc2)c2cccc(-c3cc4ccccc4cc3-c3ccc(N(c4ccccc4)c4cccc5c4c4ccccc4n5-c4ccccc4)cc3)c2)cc1 ; c1ccc(-c2cccc(N(c3ccc(-c4cc(-n5c6ccccc6c6ccccc65)cc5ccccc45)cc3)c3cccc4c3-c3ccccc3C4(c3ccccc3)c3ccccc3)c2)cc1]

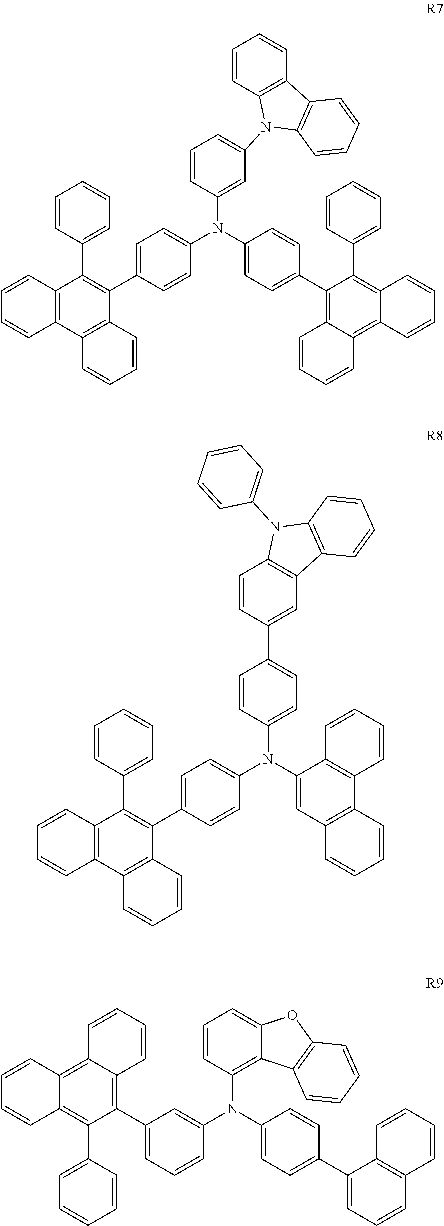 [SMILES: c1ccc(-c2c(-c3ccc(N(c4ccc(-c5c(-c6ccccc6)c6ccccc6c6ccccc56)cc4)c4cccc(-n5c6ccccc6c6ccccc65)c4)cc3)c3ccccc3c3ccccc23)cc1 ; c1ccc(-c2c(-c3ccc(N(c4ccc(-c5ccc6c(c5)c5ccccc5n6-c5ccccc5)cc4)c4cc5ccccc5c5ccccc45)cc3)c3ccccc3c3ccccc23)cc1 ; c1ccc(-c2c(-c3cccc(N(c4ccc(-c5cccc6ccccc56)cc4)c4cccc5oc6ccccc6c45)c3)c3ccccc3c3ccccc23)cc1]

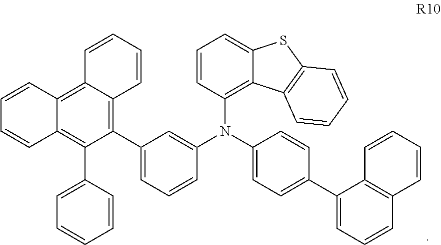 [SMILES: c1ccc(-c2c(-c3cccc(N(c4ccc(-c5cccc6ccccc56)cc4)c4cccc5sc6ccccc6c45)c3)c3ccccc3c3ccccc23)cc1]

### (2) Evaluation of Light Emitting Elements

Evaluation results of driving voltages of the light emitting elements of Examples 1 to 12, and Comparative Examples 1 to 10 are listed in Table 1. The driving voltages (%) of the light emitting elements listed in Table 1 are relative values, which are represented for comparison when it is assumed that the driving voltage of Comparative Example 1 is 100%.

TABLE 1

| | | Driving |
|---|---|---|
| | | voltage |
| Element examples | Hole transport layer material | (%) |
| Example 1 | Example Compound A9 | 94 |
| Example 2 | Example Compound A12 | 93 |
| Example 3 | Example Compound F12 | 93 |
| Example 4 | Example Compound H9 | 95 |
| Example 5 | Example Compound H12 | 95 |
| Example 6 | Example Compound N12 | 93 |
| Example 7 | Example Compound O22 | 94 |
| Example 8 | Example Compound O23 | 96 |
| Example 9 | Example Compound O32 | 94 |
| Example 10 | Example Compound P12 | 94 |
| Example 11 | Example Compound P23 | 96 |
| Example 12 | Example Compound P37 | 94 |
| Comparative Example 1 | Comparative Example Compound R1 | 100 |
| Comparative Example 2 | Comparative Example Compound R2 | 103 |
| Comparative Example 3 | Comparative Example Compound R3 | 102 |
| Comparative Example 4 | Comparative Example Compound R4 | 101 |
| Comparative Example 5 | Comparative Example Compound R5 | 99 |
| Comparative Example 6 | Comparative Example Compound R6 | 103 |
| Comparative Example 7 | Comparative Example Compound R7 | 106 |
| Comparative Example 8 | Comparative Example Compound R8 | 104 |
| Comparative Example 9 | Comparative Example Compound R9 | 105 |
| Comparative Example 10 | Comparative Example Compound R10 | 107 |

Referring to Table 1, it may be confirmed that the light emitting elements of Examples 1 to 12 have a decrease in driving voltage (%) and an improvement in element characteristics compared with the light emitting elements of Comparative Examples 1 to 10.

For example, when Example Compounds A9 and A12 are compared with Comparative Example Compounds R1 and R2, Comparative Example Compounds R1 and R2 each include a dibenzofuran group or a dibenzothiophene group, and a naphthyl group substituted with an aryl group and Example Compounds A9 and A12 each include a carbazole group and a naphthyl group substituted with an aryl group. For example, it is believed/postulated that because the amine compounds of the Examples each include the carbazole group linked to the nitrogen atom and the naphthyl group substituted with an aryl group, electronic characteristics are improved and the driving voltage is reduced.

When Example Compound F12 is compared with Comparative Example Compound R7, it is believed/postulated that because the amine compound of the Example includes a carbazole group and a naphthyl group substituted with an aryl group, each of which is linked to the nitrogen atom, and the carbazole group is directly linked to the nitrogen atom, the driving voltage is reduced. It is believed/postulated that because Comparative Example Compound R7 includes a carbazole group linked to the nitrogen atom via a phenylene group, a linker, the driving voltage is increased compared with the Example Compounds of the present disclosure.

Referring to Example Compounds A9, A12, H9, H12, and O32, it is confirmed that the electronic effect generated by including a carbazole group and a naphthyl group substituted with an aryl group as a skeleton in the amine compounds of Examples is substantially equally generated in the embodiments in which the nitrogen atom is substituted at any position selected from among the α-position and β-position of the naphthyl group.

When Example Compounds H9 and O32 are compared with Comparative Example Compound R3, it may be confirmed that the electronic effect is generated when the amine compounds of the Examples each include, as substituents, a carbazole group and a naphthyl group substituted with an aryl group and the nitrogen atom is linked to the second position or fourth position of the carbazole group. It is believed/postulated that because the Comparative Example Compound includes, as substituents, a carbazole group and a naphthyl group substituted with an aryl group and the nitrogen atom is linked to the third position of the carbazole group, the driving voltage of the element is increased. This is believed/postulated to be due to the effect of the linkage position between the carbazole group and the nitrogen atom.

When Example Compound N12 is compared with Comparative Example Compound R5, it is believed/postulated that because the amine compound of the Example includes, as substituents, a carbazole group and a naphthyl group substituted with an aryl group, each of which is linked to the nitrogen atom, and this satisfies a monoamine structure, the driving voltage is reduced. For Comparative Example Compound R5, it is believed/postulated that because the amine compound includes a carbazole group and a naphthyl group substituted with an aryl group, but includes a naphthyl group substituted with a diphenyl amine group, and has a diamine compound structure, the driving voltage is increased compared with the Example Compounds of the present disclosure.

When Example Compounds O22 and O23 are compared with Comparative Example Compound R4, it is believed/postulated that the amine compounds of the Examples have a reduction in driving voltage and an improvement in element characteristics because when the nitrogen atom is substituted at the fourth position of the carbazole group and is also linked to the third or fourth position of the fluorene group, a steric effect due to the fluorene group is generated. In contrast, for Comparative Example Compound R4, the nitrogen atom is substituted at the fourth position of the carbazole group, but the nitrogen atom is linked to the second position of the fluorene group. In this embodiment, in the amine compound, the carbazole group, the naphthyl group substituted with an aryl group, and the fluorene group are closer to each other, and thus the molecule is further twisted, and the resistance to redox is reduced, and thus the driving voltage may be increased.

When Example Compounds P12, P23, and P37 are compared with Comparative Example Compounds R8 to R10, it is confirmed that even when the amine compounds of the Examples each include a carbazole group directly linked to the nitrogen atom and a phenanthryl group substituted with an aryl group, the phenanthryl group being linked to the nitrogen atom via a linker, the effect of reducing the driving voltage is generated. This is believed to be caused by the electronic effect between the carbazole group and the phenanthryl group substituted with an aryl group. It is believed that Comparative Examples Compounds R8 to R10 represent a structure in which a phenanthryl group substituted with an aryl group is linked to the nitrogen atom of an amine group, but do not represent a carbazole group linked to the nitrogen atom, or the carbazole group is not directly linked to the nitrogen atom, thereby increasing the driving voltage as compared with the Example Compounds of the present disclosure.

For example, when Example Compounds P12, P23, and P37 are compared with Comparative Example Compounds R7 and R8, it may be confirmed that when the amine compounds of the Examples each include a carbazole group and a phenanthryl group, each of which is linked to the nitrogen atom, the effect of suppressing the driving voltage is generated regardless of the substitution position of the nitrogen atom and the carbazole group. It may be postulated that this is because the phenanthryl group has electrons more easily delocalized than the naphthyl group to thus enhance a resonance structure, and that the electronic effect between the carbazole group and the phenanthryl group is increased more than the electronic effect between the carbazole group and the naphthyl group. However, it is believed that this is limited to the embodiment in which a carbazole group is directly linked to the nitrogen atom. For Comparative Example Compounds R7 and R₈, it is believed that because the amine compounds each include a carbazole group and a phenanthryl group, each of which is linked to the nitrogen atom, but the carbazole group is not directly linked to the nitrogen atom, the driving voltage is increased compared with the Example Compounds of present disclosure.

With regard to this, referring to the aforementioned Example Compounds O22 and O23 and Comparative Example Compound R4, when the amine compound of the present disclosure includes a carbazole group, a naphthyl group, and a fluorene group, each of which is linked to the nitrogen atom of the amine group, the effect of suppressing the driving voltage is optionally generated according to a linkage position between the nitrogen atom and the fluorene group. For example, when a naphthyl group and a fluorene group, each of which is linked to the nitrogen atom of the amine group, are included, the effect of reducing the driving voltage is generated when the nitrogen atom is linked to the third or fourth position of the fluorene group, and in contrast, the effect of suppressing the driving voltage is not generated when the nitrogen atom is linked to the second position of the fluorene group. It is believed that this is because the bulky molecule, such as a fluorene group, has an effect on the intermolecular orientation in the amine compound.

However, referring to Example Compounds P12, P23, and P37, when the amine compound of the present disclosure includes a carbazole group, a phenanthryl group, and a fluorene group, each of which is linked to the nitrogen atom of the amine group, it is believed that because the phenanthryl group has enhanced delocalization and resonance structure of electrons compared with the naphthyl group, and thus stacking between the carbazole group and the phenanthryl group is more effective than stacking between the carbazole group and the naphthyl group, and the effect on the intermolecular orientation in the amine compound is reduced, and thus the electronic effect between the carbazole group and the phenanthryl group is generated without being limited to the linkage position of the nitrogen atom and the fluorene group, thereby reducing the driving voltage of the element.

The amine compound of the present disclosure has a monoamine compound structure, and includes a first substituent that is a carbazole group, a second substituent that is a naphthyl group substituted with an aryl group, and a third substituent that is an aryl group or a heteroaryl group, each of which is linked to the nitrogen atom. The carbazole group, which is the first substituent, is directly bonded to the nitrogen atom to thus easily supply and transport holes between the first substituent and the second substituent, thereby improving the material stability and hole transport property of the amine compound. In some embodiments, the second substituent may be a naphthyl group, or may have a phenanthryl group structure, and when the second substituent is a phenanthryl group, the electronic interaction between the carbazole group and the second substituent may be further increased.

The light emitting element of the present disclosure includes the amine compound in the hole transport region, and thus the hole transport property may be enhanced and the driving voltage may be reduced, thereby improving element characteristics.

The display device of the present disclosure includes the light emitting element including the amine compound, and thus the driving voltage of the display device may be reduced.

The amine compound of an embodiment may have an improvement in the stability of material, and thus may be used as a material for implementing the light emitting element having low driving voltage.

The light emitting element of an embodiment and the display device including the same may exhibit low driving voltage characteristics.

The use of “may” when describing embodiments of the present disclosure refers to “one or more embodiments of the present disclosure.”

As used herein, the term “substantially,” “about,” and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this disclosure is intended to include all higher numerical limitations subsumed therein. Accordingly, Applicant reserves the right to amend this disclosure, including the claims, to expressly recite any sub-range subsumed within the ranges expressly recited herein.

The light emitting device or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the embodiments of the present disclosure have been described, it is understood that the present disclosure should not be limited to these embodiments, but one or more suitable changes and modifications can be made by one ordinary skilled in the art within the spirit and scope of the present present disclosure as defined by the following claims and equivalents thereof.

What is claimed is:

1. A light emitting element comprising:
a first electrode;
a second electrode on the first electrode;
an emission layer between the first electrode and the second electrode; and
a hole transport region which is between the first electrode and the emission layer and comprises an amine compound represented by Formula 1: 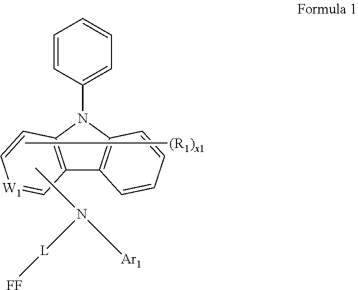
wherein, in Formula 1,
Ar₁ is a substituted or unsubstituted aryl group having 6 to 50 ring-forming carbon atoms, or a substituted or unsubstituted heteroaryl group having 2 to 50 ring-forming carbon atoms, and Ar₁ is not a substituted or unsubstituted carbazole group,
L is a substituted or unsubstituted arylene group having 6 to 50 ring-forming carbon atoms, or a substituted or unsubstituted heteroarylene group having 2 to 50 ring-forming carbon atoms,
R₁ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or are bonded to an adjacent group to form a ring,
x1 is an integer from 0 to 5,
W₁ and W₂ are each independently CRₐ or a carbon atom bonded to the nitrogen atom in Formula 1,
Rₐ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring,
FF is represented by Formula 2, and
in Formula 2, —* is a position linked to L, and 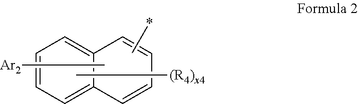
wherein, in Formula 2,
Ar₂ is a substituted or unsubstituted aryl group having 6 to 50 ring-forming carbon atoms, and Ar₂ is not an aryl group substituted with an amine group,
R₄ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, and
x4 is an integer from 0 to 6, and
wherein, when R₄ does not form a ring with an adjacent group, each of W₁ and W₂ in Formula 1 is CRₐ, and 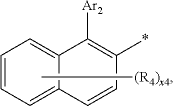
wherein, when Formula 2 is represented by R₄ does not form a ring with an adjacent group, and Ar₁ is a substituted or unsubstituted fluorene group, and the nitrogen atom in Formula 1 is not linked to the second position of the substituted or unsubstituted fluorene group, Ar₁.

2. The light emitting element of claim 1, wherein FF is represented by Formula 2a: 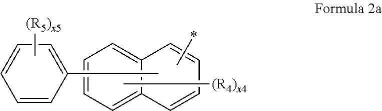
wherein, in Formula 2a,
R₅ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring,
x5 is an integer from 0 to 5, and
R₄ and x4 are the same as defined in Formula 2.

3. The light emitting element of claim 1, wherein FF is represented by any one selected from among Formula 2-1 to Formula 2-5: 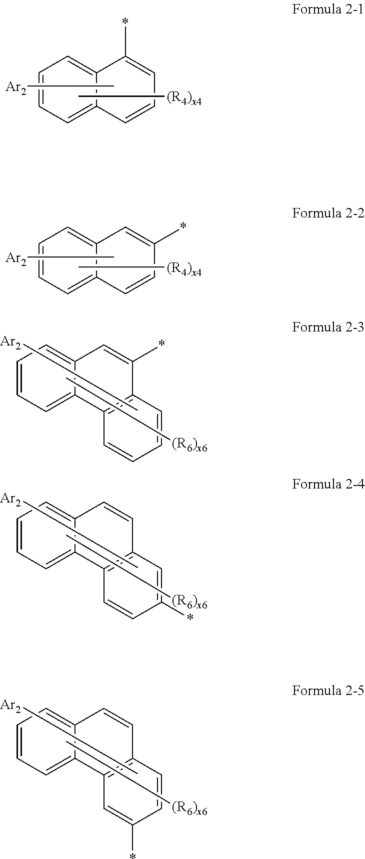
wherein, in Formula 2-3 to Formula 2-5,
R₆ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, and
x6 is an integer from 0 to 8, and
wherein Formula 2-1 to Formula 2-5, Ar₂, R₄, and x4 are the same as defined in Formula 2.

4. The light emitting element of claim 3, wherein FF is represented by any one selected from among Formula 2-3a, Formula 2-4a, and Formula 2-5a: 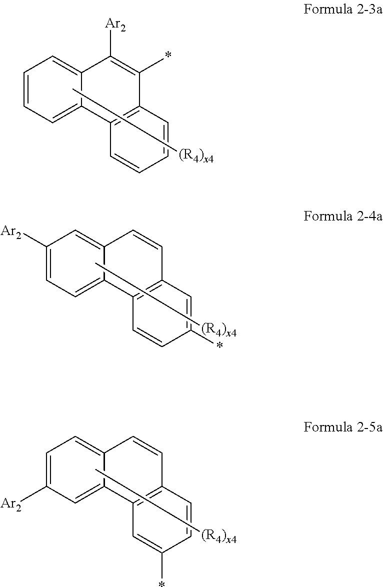
wherein, in Formula 2-3a, Formula 2-4a, and Formula 2-5a, Ar₂, R₄, and x4 are the same as defined in Formula 2.

5. The light emitting element of claim 1, wherein the amine compound represented by Formula 1 is represented by any one selected from among Formula 3-1 to Formula 3-3: 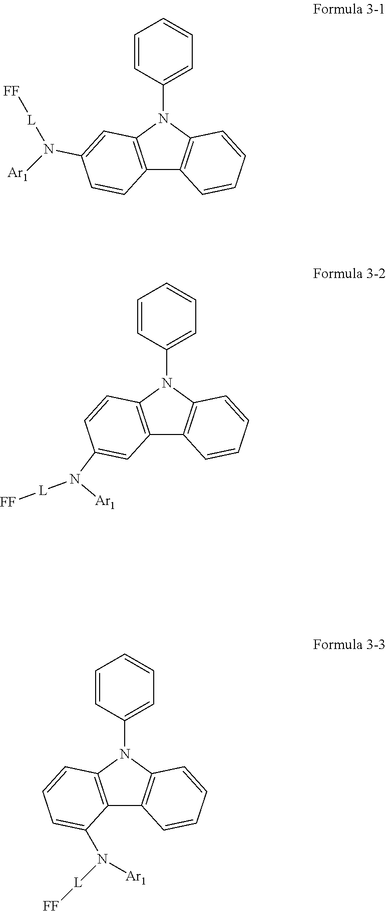
wherein, in Formula 3-1 to Formula 3-3, L, Ar₁, and FF are the same as defined in Formula 1.

6. The light emitting element of claim 1, wherein L is a substituted or unsubstituted phenylene group, a substituted or unsubstituted biphenylene group, or a substituted or unsubstituted divalent dibenzofuran group.

7. The light emitting element of claim 1, wherein Ar₁ is a substituted or unsubstituted phenyl group, a substituted or unsubstituted biphenyl group, a substituted or unsubstituted terphenyl group, a substituted or unsubstituted quaterphenyl group, a substituted or unsubstituted fluorenyl group, a substituted or unsubstituted naphthyl group, a substituted or unsubstituted phenanthryl group, a substituted or unsubstituted dibenzofuran group, or a substituted or unsubstituted dibenzothiophene group.

8. The light emitting element of claim 1, wherein the hole transport region further comprises a compound represented by Formula H-1: 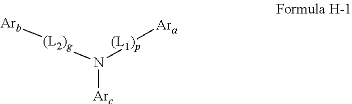
wherein, in Formula H-1,
Arₐ and Ar_{b} are each independently a substituted or unsubstituted aryl group having 6 to 30 ring-forming carbon atoms, or a substituted or unsubstituted heteroaryl group having 2 to 30 ring-forming carbon atoms,
Ar_{c} is a substituted or unsubstituted aryl group having 6 to 30 ring-forming carbon atoms,
L₁ and L₂ are each independently a direct linkage, a substituted or unsubstituted arylene group having 6 to 30 ring-forming carbon atoms, or a substituted or unsubstituted heteroarylene group having 2 to 30 ring-forming carbon atoms, and
p and q are each independently an integer from 0 to 10.

9. The light emitting element of claim 1, wherein the emission layer comprises a compound represented by Formula E-1: 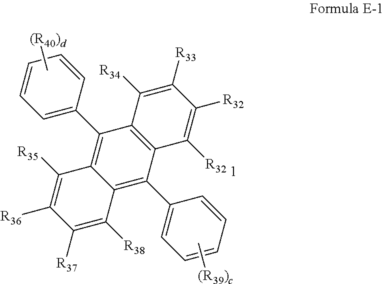
wherein, in Formula E-1,
R₃₁ to R₄₀ are each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring, and
c and d are each independently an integer from 0 to 5.

10. The light emitting element of claim 1, wherein the hole transport region comprises a hole injection layer on the first electrode, a hole transport layer on the hole injection layer, and an electron blocking layer on the hole transport layer, and
the hole transport layer comprises the amine compound represented by Formula 1.

11. The light emitting element of claim 1, wherein the amine compound is a monoamine compound.

12. The light emitting element of claim 1, wherein the amine compound represented by Formula 1 comprises at least one selected from among compounds represented by Compound Group 1, FF—* in Compound Group 1 is linked to *—L, and each of 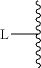
and 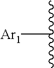
is linked to 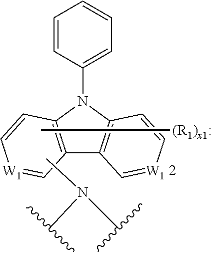
Compound Group 1

| Divi- sion | 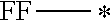 | 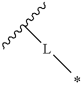 | 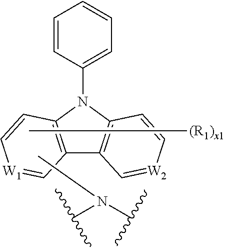 | 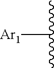 |
|---|---|---|---|---|
| A1 | 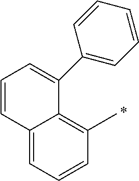 [SMILES: Cc1cccc2cccc(-c3ccccc3)c12] | 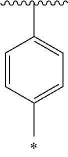 [SMILES: *c1ccc(C)cc1] | 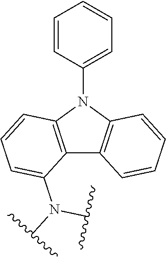 [SMILES: *N(*)c1cccc2c1c1ccccc1n2-c1ccccc1] | 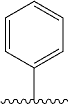 [SMILES: *c1ccccc1] |
| A2 | 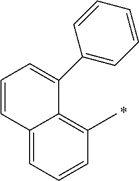 [SMILES: Cc1cccc2cccc(-c3ccccc3)c12] | 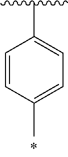 [SMILES: *c1ccc(C)cc1] | 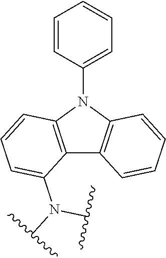 [SMILES: *N(*)c1cccc2c1c1ccccc1n2-c1ccccc1] | 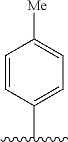 [SMILES: *c1ccc(C)cc1] |
| A3 | 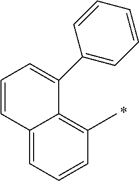 [SMILES: Cc1cccc2cccc(-c3ccccc3)c12] | 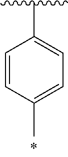 [SMILES: *c1ccc(C)cc1] | 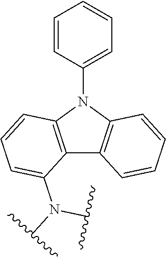 [SMILES: *N(*)c1cccc2c1c1ccccc1n2-c1ccccc1] | 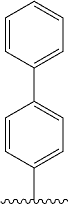 [SMILES: *c1ccc(-c2ccccc2)cc1] |
| A4 | 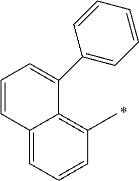 [SMILES: Cc1cccc2cccc(-c3ccccc3)c12] | 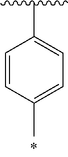 [SMILES: *c1ccc(C)cc1] | 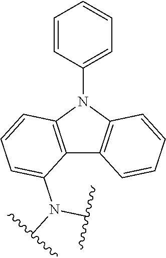 [SMILES: *N(*)c1cccc2c1c1ccccc1n2-c1ccccc1] | 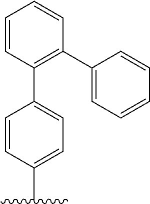 [SMILES: *c1ccc(-c2ccccc2-c2ccccc2)cc1] |
| A5 | 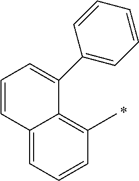 [SMILES: Cc1cccc2cccc(-c3ccccc3)c12] | 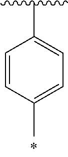 [SMILES: *c1ccc(C)cc1] | 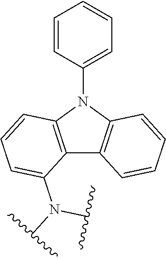 [SMILES: *N(*)c1cccc2c1c1ccccc1n2-c1ccccc1] | 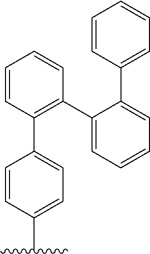 [SMILES: *c1ccc(-c2ccccc2-c2ccccc2-c2ccccc2)cc1] |
| A6 | 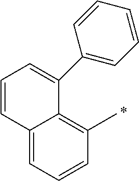 [SMILES: Cc1cccc2cccc(-c3ccccc3)c12] | 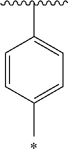 [SMILES: *c1ccc(C)cc1] | 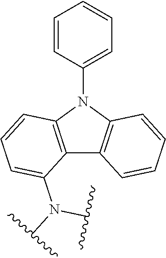 [SMILES: *N(*)c1cccc2c1c1ccccc1n2-c1ccccc1] | 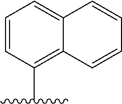 [SMILES: *c1cccc2ccccc12] |
| A7 | 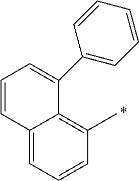 [SMILES: Cc1cccc2cccc(-c3ccccc3)c12] | 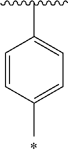 [SMILES: *c1ccc(C)cc1] | 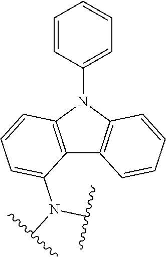 [SMILES: *N(*)c1cccc2c1c1ccccc1n2-c1ccccc1] | 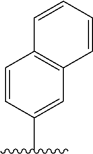 [SMILES: *c1ccc2ccccc2c1] |
| A8 | 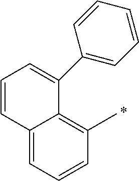 [SMILES: Cc1cccc2cccc(-c3ccccc3)c12] | 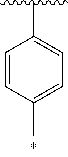 [SMILES: *c1ccc(C)cc1] | 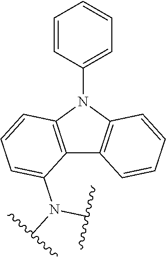 [SMILES: *N(*)c1cccc2c1c1ccccc1n2-c1ccccc1] | 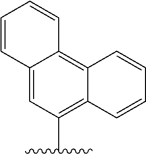 [SMILES: *c1cc2ccccc2c2ccccc12] |
| A9 | 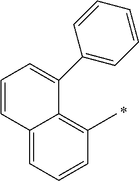 [SMILES: Cc1cccc2cccc(-c3ccccc3)c12] | 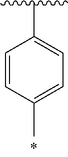 [SMILES: *c1ccc(C)cc1] | 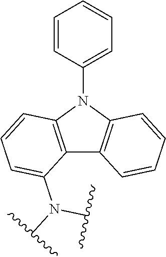 [SMILES: *N(*)c1cccc2c1c1ccccc1n2-c1ccccc1] | 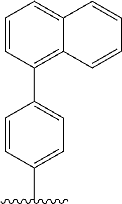 [SMILES: *c1ccc(-c2cccc3ccccc23)cc1] |
| A10 | 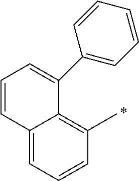 [SMILES: Cc1cccc2cccc(-c3ccccc3)c12] | 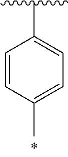 [SMILES: *c1ccc(C)cc1] | 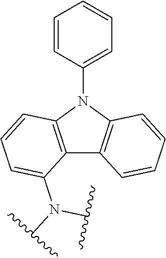 [SMILES: *N(*)c1cccc2c1c1ccccc1n2-c1ccccc1] | 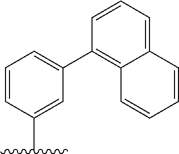 [SMILES: *c1cccc(-c2cccc3ccccc23)c1] |
| A11 | 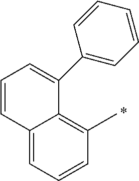 [SMILES: Cc1cccc2cccc(-c3ccccc3)c12] | 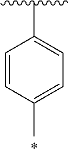 [SMILES: *c1ccc(C)cc1] | 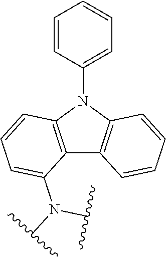 [SMILES: *N(*)c1cccc2c1c1ccccc1n2-c1ccccc1] | 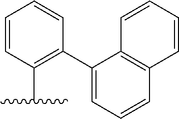 [SMILES: *c1ccccc1-c1cccc2ccccc12] |
| A12 | 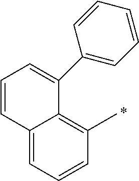 [SMILES: Cc1cccc2cccc(-c3ccccc3)c12] | 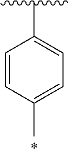 [SMILES: *c1ccc(C)cc1] | 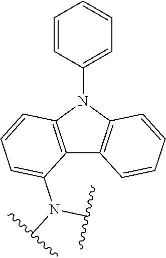 [SMILES: *N(*)c1cccc2c1c1ccccc1n2-c1ccccc1] | 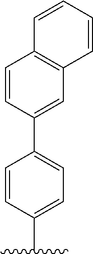 [SMILES: *c1ccc(-c2ccc3ccccc3c2)cc1] |
| A13 | 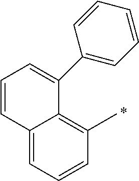 [SMILES: Cc1cccc2cccc(-c3ccccc3)c12] | 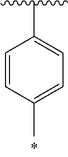 [SMILES: *c1ccc(C)cc1] | 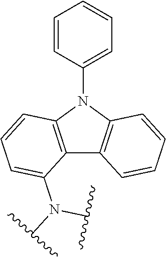 [SMILES: *N(*)c1cccc2c1c1ccccc1n2-c1ccccc1] | 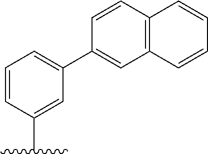 [SMILES: *c1cccc(-c2ccc3ccccc3c2)c1] |
| A14 | 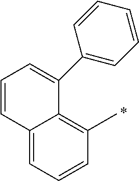 [SMILES: Cc1cccc2cccc(-c3ccccc3)c12] | 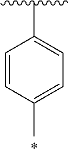 [SMILES: *c1ccc(C)cc1] | 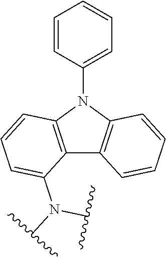 [SMILES: *N(*)c1cccc2c1c1ccccc1n2-c1ccccc1] | 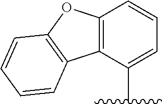 [SMILES: *c1cccc2oc3ccccc3c12] |
| A15 | 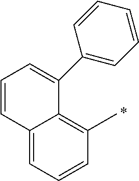 [SMILES: Cc1cccc2cccc(-c3ccccc3)c12] | 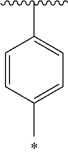 [SMILES: *c1ccc(C)cc1] | 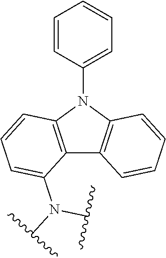 [SMILES: *N(*)c1cccc2c1c1ccccc1n2-c1ccccc1] | 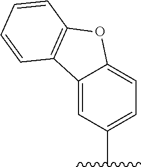 [SMILES: *c1ccc2oc3ccccc3c2c1] |
| A16 | 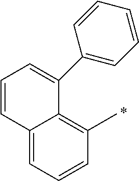 [SMILES: Cc1cccc2cccc(-c3ccccc3)c12] | 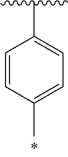 [SMILES: *c1ccc(C)cc1] | 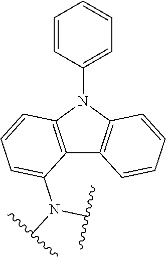 [SMILES: *N(*)c1cccc2c1c1ccccc1n2-c1ccccc1] | 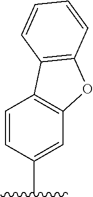 [SMILES: *c1ccc2c(c1)oc1ccccc12] |
| A17 | 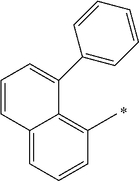 [SMILES: Cc1cccc2cccc(-c3ccccc3)c12] | 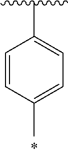 [SMILES: *c1ccc(C)cc1] | 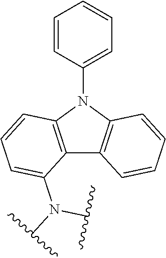 [SMILES: *N(*)c1cccc2c1c1ccccc1n2-c1ccccc1] | 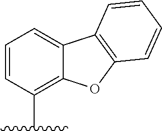 [SMILES: *c1cccc2c1oc1ccccc12] |
| A18 | 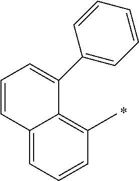 [SMILES: Cc1cccc2cccc(-c3ccccc3)c12] | 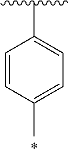 [SMILES: *c1ccc(C)cc1] | 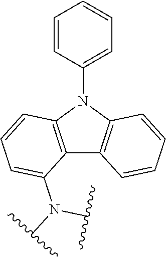 [SMILES: *N(*)c1cccc2c1c1ccccc1n2-c1ccccc1] | 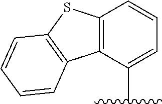 [SMILES: *c1cccc2sc3ccccc3c12] |
| A19 | 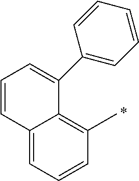 [SMILES: Cc1cccc2cccc(-c3ccccc3)c12] | 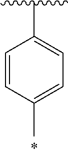 [SMILES: *c1ccc(C)cc1] | 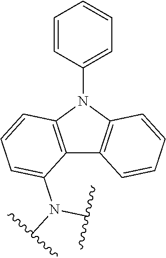 [SMILES: *N(*)c1cccc2c1c1ccccc1n2-c1ccccc1] | 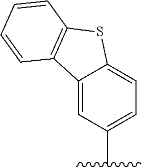 [SMILES: *c1ccc2sc3ccccc3c2c1] |
| A20 | 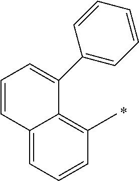 [SMILES: Cc1cccc2cccc(-c3ccccc3)c12] | 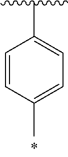 [SMILES: *c1ccc(C)cc1] | 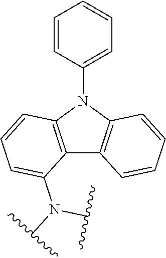 [SMILES: *N(*)c1cccc2c1c1ccccc1n2-c1ccccc1] | 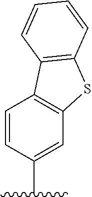 [SMILES: *c1ccc2c(c1)sc1ccccc12] |
| A21 | 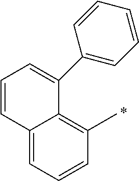 [SMILES: Cc1cccc2cccc(-c3ccccc3)c12] | 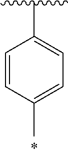 [SMILES: *c1ccc(C)cc1] | 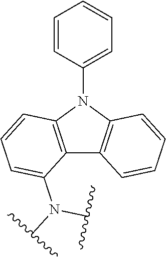 [SMILES: *N(*)c1cccc2c1c1ccccc1n2-c1ccccc1] | 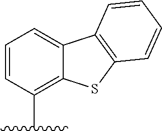 [SMILES: *c1cccc2c1sc1ccccc12] |
| A22 | 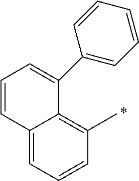 [SMILES: Cc1cccc2cccc(-c3ccccc3)c12] | 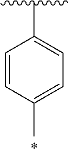 [SMILES: *c1ccc(C)cc1] | 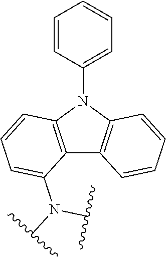 [SMILES: *N(*)c1cccc2c1c1ccccc1n2-c1ccccc1] | 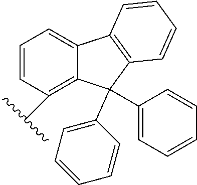 [SMILES: *c1cccc2c1C(c1ccccc1)(c1ccccc1)c1ccccc1-2] |
| A23 | 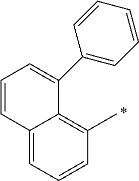 [SMILES: Cc1cccc2cccc(-c3ccccc3)c12] | 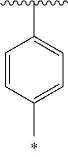 [SMILES: *c1ccc(C)cc1] | 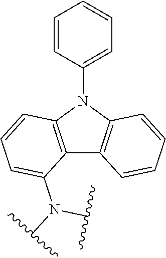 [SMILES: *N(*)c1cccc2c1c1ccccc1n2-c1ccccc1] | 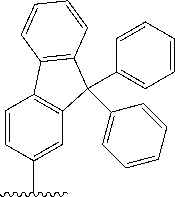 [SMILES: *c1ccc2c(c1)C(c1ccccc1)(c1ccccc1)c1ccccc1-2] |
| A24 | 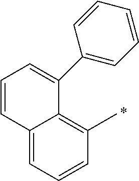 [SMILES: Cc1cccc2cccc(-c3ccccc3)c12] | 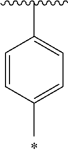 [SMILES: *c1ccc(C)cc1] | 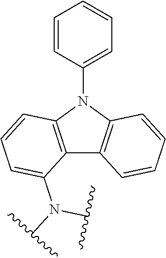 [SMILES: *N(*)c1cccc2c1c1ccccc1n2-c1ccccc1] | 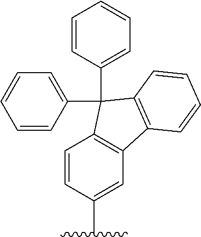 [SMILES: *c1ccc2c(c1)-c1ccccc1C2(c1ccccc1)c1ccccc1] |
| A25 | 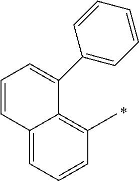 [SMILES: Cc1cccc2cccc(-c3ccccc3)c12] | 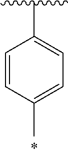 [SMILES: *c1ccc(C)cc1] | 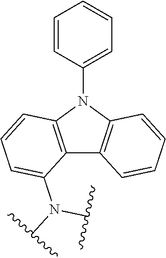 [SMILES: *N(*)c1cccc2c1c1ccccc1n2-c1ccccc1] | 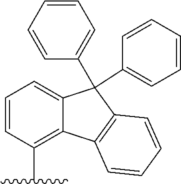 [SMILES: *c1cccc2c1-c1ccccc1C2(c1ccccc1)c1ccccc1] |
| A26 | 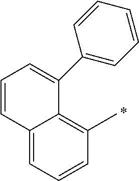 [SMILES: Cc1cccc2cccc(-c3ccccc3)c12] | 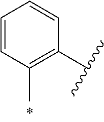 [SMILES: *c1ccccc1C] | 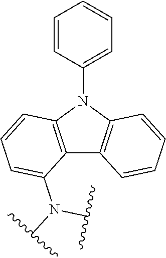 [SMILES: *N(*)c1cccc2c1c1ccccc1n2-c1ccccc1] | 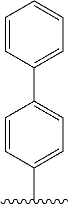 [SMILES: *c1ccc(-c2ccccc2)cc1] |
| A27 | 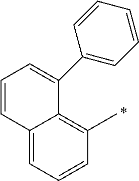 [SMILES: Cc1cccc2cccc(-c3ccccc3)c12] | 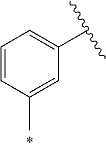 [SMILES: *c1cccc(C)c1] | 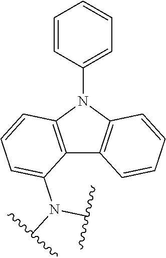 [SMILES: *N(*)c1cccc2c1c1ccccc1n2-c1ccccc1] | 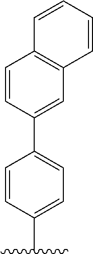 [SMILES: *c1ccc(-c2ccc3ccccc3c2)cc1] |
| A28 | 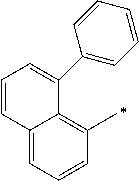 [SMILES: Cc1cccc2cccc(-c3ccccc3)c12] | 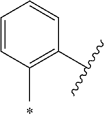 [SMILES: *c1ccccc1C] | 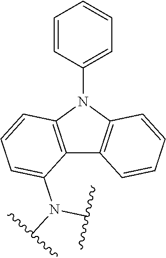 [SMILES: *N(*)c1cccc2c1c1ccccc1n2-c1ccccc1] | 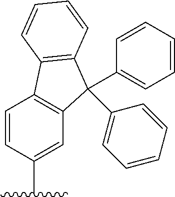 [SMILES: *c1ccc2c(c1)C(c1ccccc1)(c1ccccc1)c1ccccc1-2] |
| A29 | 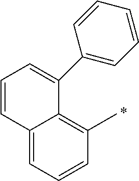 [SMILES: Cc1cccc2cccc(-c3ccccc3)c12] | 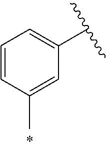 [SMILES: *c1cccc(C)c1] | 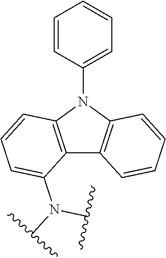 [SMILES: *N(*)c1cccc2c1c1ccccc1n2-c1ccccc1] | 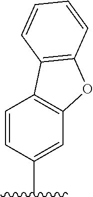 [SMILES: *c1ccc2c(c1)oc1ccccc12] |
| A30 | 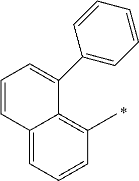 [SMILES: Cc1cccc2cccc(-c3ccccc3)c12] | 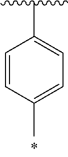 [SMILES: *c1ccc(C)cc1] | 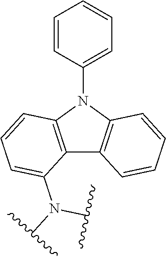 [SMILES: *N(*)c1cccc2c1c1ccccc1n2-c1ccccc1] | 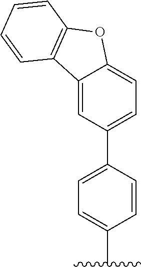 [SMILES: *c1ccc(-c2ccc3oc4ccccc4c3c2)cc1] |
| A31 | 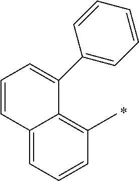 [SMILES: Cc1cccc2cccc(-c3ccccc3)c12] | 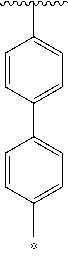 [SMILES: *c1ccc(-c2ccc(C)cc2)cc1] | 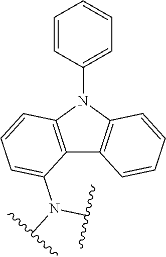 [SMILES: *N(*)c1cccc2c1c1ccccc1n2-c1ccccc1] | 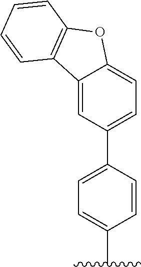 [SMILES: *c1ccc(-c2ccc3oc4ccccc4c3c2)cc1] |
| A32 | 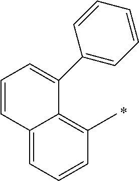 [SMILES: Cc1cccc2cccc(-c3ccccc3)c12] | 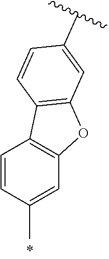 [SMILES: *c1ccc2c(c1)oc1cc(C)ccc12] | 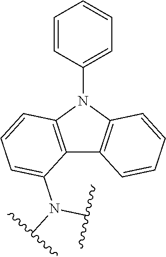 [SMILES: *N(*)c1cccc2c1c1ccccc1n2-c1ccccc1] | 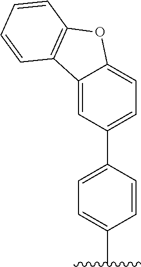 [SMILES: *c1ccc(-c2ccc3oc4ccccc4c3c2)cc1] |
| A33 | 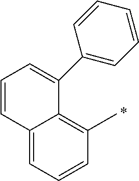 [SMILES: Cc1cccc2cccc(-c3ccccc3)c12] | 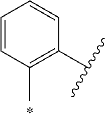 [SMILES: *c1ccccc1C] | 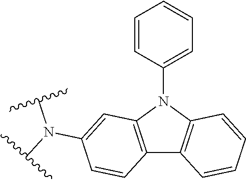 [SMILES: *N(*)c1ccc2c3ccccc3n(-c3ccccc3)c2c1] | 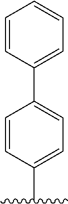 [SMILES: *c1ccc(-c2ccccc2)cc1] |
| A34 | 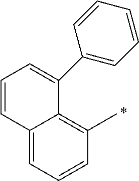 [SMILES: Cc1cccc2cccc(-c3ccccc3)c12] | 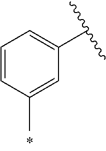 [SMILES: *c1cccc(C)c1] | 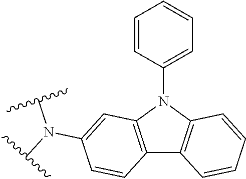 [SMILES: *N(*)c1ccc2c3ccccc3n(-c3ccccc3)c2c1] | 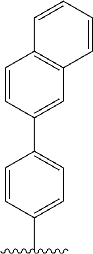 [SMILES: *c1ccc(-c2ccc3ccccc3c2)cc1] |
| A35 | 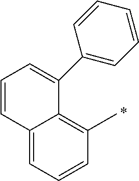 [SMILES: Cc1cccc2cccc(-c3ccccc3)c12] | 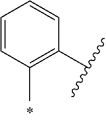 [SMILES: *c1ccccc1C] | 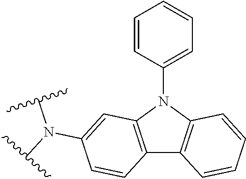 [SMILES: *N(*)c1ccc2c3ccccc3n(-c3ccccc3)c2c1] | 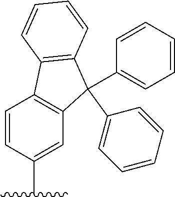 [SMILES: *c1ccc2c(c1)C(c1ccccc1)(c1ccccc1)c1ccccc1-2] |
| A36 | 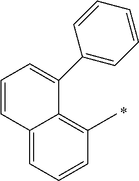 [SMILES: Cc1cccc2cccc(-c3ccccc3)c12] | 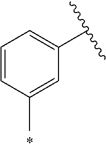 [SMILES: *c1cccc(C)c1] | 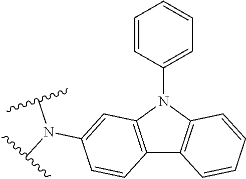 [SMILES: *N(*)c1ccc2c3ccccc3n(-c3ccccc3)c2c1] | 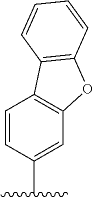 [SMILES: *c1ccc2c(c1)oc1ccccc12] |
| A37 | 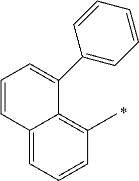 [SMILES: Cc1cccc2cccc(-c3ccccc3)c12] | 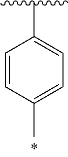 [SMILES: *c1ccc(C)cc1] | 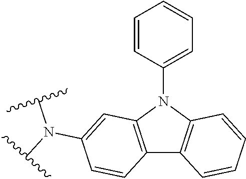 [SMILES: *N(*)c1ccc2c3ccccc3n(-c3ccccc3)c2c1] | 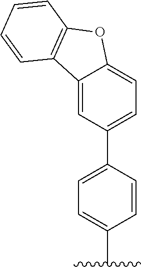 [SMILES: *c1ccc(-c2ccc3oc4ccccc4c3c2)cc1] |
| A38 | 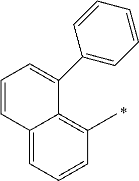 [SMILES: Cc1cccc2cccc(-c3ccccc3)c12] | 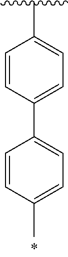 [SMILES: *c1ccc(-c2ccc(C)cc2)cc1] | 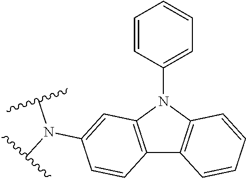 [SMILES: *N(*)c1ccc2c3ccccc3n(-c3ccccc3)c2c1] | 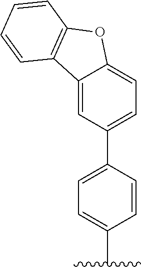 [SMILES: *c1ccc(-c2ccc3oc4ccccc4c3c2)cc1] |
| A39 | 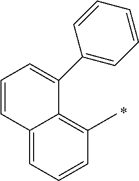 [SMILES: Cc1cccc2cccc(-c3ccccc3)c12] | 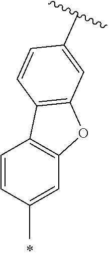 [SMILES: *c1ccc2c(c1)oc1cc(C)ccc12] | 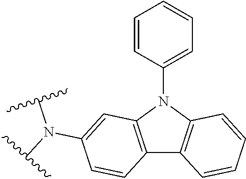 [SMILES: *N(*)c1ccc2c3ccccc3n(-c3ccccc3)c2c1] | 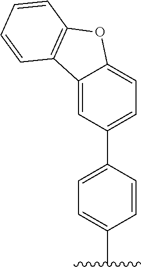 [SMILES: *c1ccc(-c2ccc3oc4ccccc4c3c2)cc1] |
| B1 | 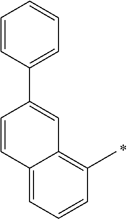 [SMILES: Cc1cccc2ccc(-c3ccccc3)cc12] | 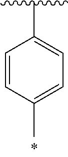 [SMILES: *c1ccc(C)cc1] | 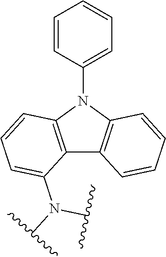 [SMILES: *N(*)c1cccc2c1c1ccccc1n2-c1ccccc1] | 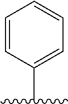 [SMILES: *c1ccccc1] |
| B2 | 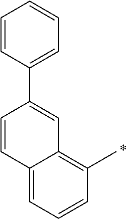 [SMILES: Cc1cccc2ccc(-c3ccccc3)cc12] | 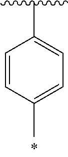 [SMILES: *c1ccc(C)cc1] | 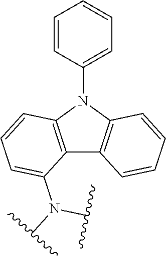 [SMILES: *N(*)c1cccc2c1c1ccccc1n2-c1ccccc1] | 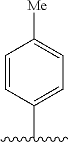 [SMILES: *c1ccc(C)cc1] |
| B3 | 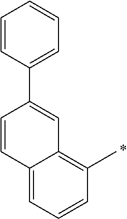 [SMILES: Cc1cccc2ccc(-c3ccccc3)cc12] | 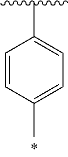 [SMILES: *c1ccc(C)cc1] | 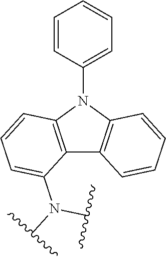 [SMILES: *N(*)c1cccc2c1c1ccccc1n2-c1ccccc1] | 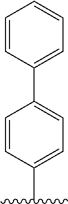 [SMILES: *c1ccc(-c2ccccc2)cc1] |
| B4 | 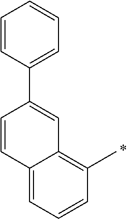 [SMILES: Cc1cccc2ccc(-c3ccccc3)cc12] | 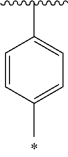 [SMILES: *c1ccc(C)cc1] | 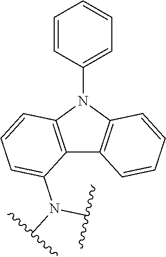 [SMILES: *N(*)c1cccc2c1c1ccccc1n2-c1ccccc1] | 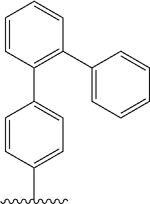 [SMILES: *c1ccc(-c2ccccc2-c2ccccc2)cc1] |
| B5 | 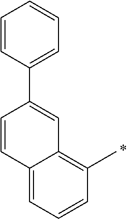 [SMILES: Cc1cccc2ccc(-c3ccccc3)cc12] | 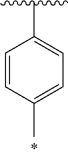 [SMILES: *c1ccc(C)cc1] | 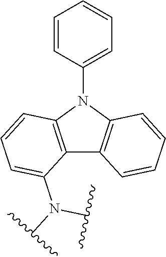 [SMILES: *N(*)c1cccc2c1c1ccccc1n2-c1ccccc1] | 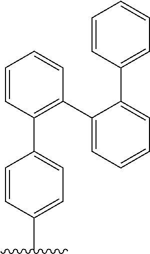 [SMILES: *c1ccc(-c2ccccc2-c2ccccc2-c2ccccc2)cc1] |
| B6 | 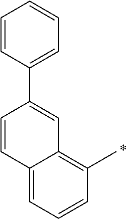 [SMILES: Cc1cccc2ccc(-c3ccccc3)cc12] | 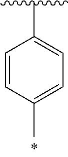 [SMILES: *c1ccc(C)cc1] | 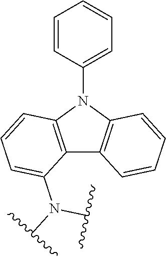 [SMILES: *N(*)c1cccc2c1c1ccccc1n2-c1ccccc1] | 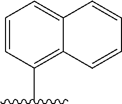 [SMILES: *c1cccc2ccccc12] |
| B7 | 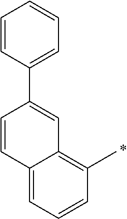 [SMILES: Cc1cccc2ccc(-c3ccccc3)cc12] | 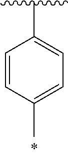 [SMILES: *c1ccc(C)cc1] | 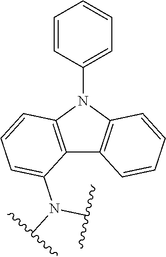 [SMILES: *N(*)c1cccc2c1c1ccccc1n2-c1ccccc1] | 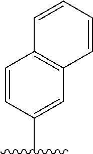 [SMILES: *c1ccc2ccccc2c1] |
| B8 | 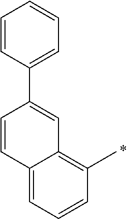 [SMILES: Cc1cccc2ccc(-c3ccccc3)cc12] | 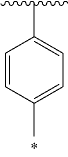 [SMILES: *c1ccc(C)cc1] | 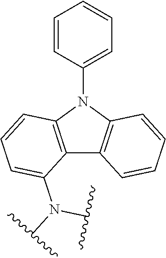 [SMILES: *N(*)c1cccc2c1c1ccccc1n2-c1ccccc1] | 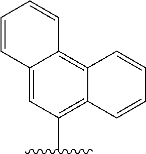 [SMILES: *c1cc2ccccc2c2ccccc12] |
| B9 | 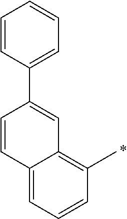 [SMILES: Cc1cccc2ccc(-c3ccccc3)cc12] | 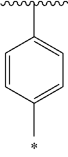 [SMILES: *c1ccc(C)cc1] | 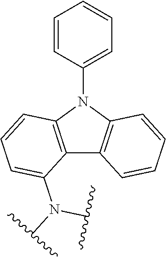 [SMILES: *N(*)c1cccc2c1c1ccccc1n2-c1ccccc1] | 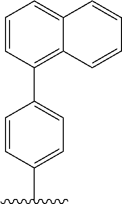 [SMILES: *c1ccc(-c2cccc3ccccc23)cc1] |
| B10 | 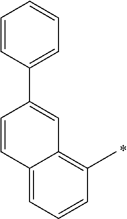 [SMILES: Cc1cccc2ccc(-c3ccccc3)cc12] | 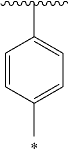 [SMILES: *c1ccc(C)cc1] | 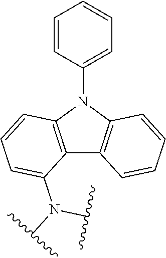 [SMILES: *N(*)c1cccc2c1c1ccccc1n2-c1ccccc1] | 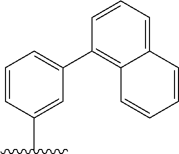 [SMILES: *c1cccc(-c2cccc3ccccc23)c1] |
| B11 | 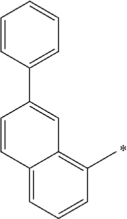 [SMILES: Cc1cccc2ccc(-c3ccccc3)cc12] | 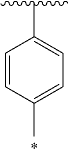 [SMILES: *c1ccc(C)cc1] | 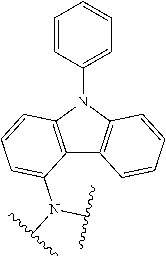 [SMILES: *N(*)c1cccc2c1c1ccccc1n2-c1ccccc1] | 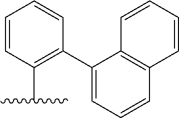 [SMILES: *c1ccccc1-c1cccc2ccccc12] |
| B12 | 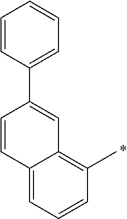 [SMILES: Cc1cccc2ccc(-c3ccccc3)cc12] | 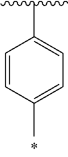 [SMILES: *c1ccc(C)cc1] | 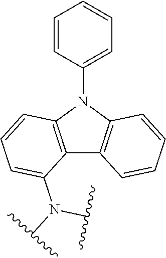 [SMILES: *N(*)c1cccc2c1c1ccccc1n2-c1ccccc1] | 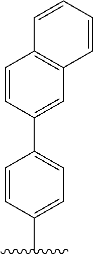 [SMILES: *c1ccc(-c2ccc3ccccc3c2)cc1] |
| B13 | 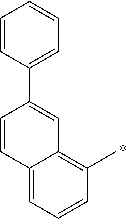 [SMILES: Cc1cccc2ccc(-c3ccccc3)cc12] | 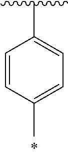 [SMILES: *c1ccc(C)cc1] | 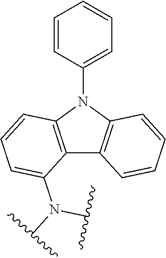 [SMILES: *N(*)c1cccc2c1c1ccccc1n2-c1ccccc1] | 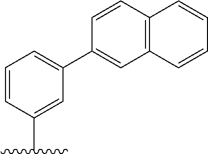 [SMILES: *c1cccc(-c2ccc3ccccc3c2)c1] |
| B14 | 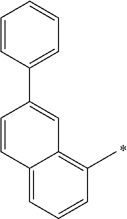 [SMILES: Cc1cccc2ccc(-c3ccccc3)cc12] | 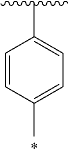 [SMILES: *c1ccc(C)cc1] | 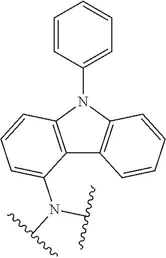 [SMILES: *N(*)c1cccc2c1c1ccccc1n2-c1ccccc1] | 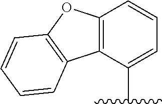 [SMILES: *c1cccc2oc3ccccc3c12] |
| B15 | 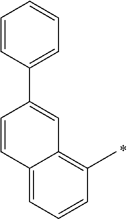 [SMILES: Cc1cccc2ccc(-c3ccccc3)cc12] | 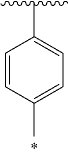 [SMILES: *c1ccc(C)cc1] | 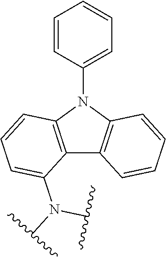 [SMILES: *N(*)c1cccc2c1c1ccccc1n2-c1ccccc1] | 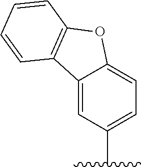 [SMILES: *c1ccc2oc3ccccc3c2c1] |
| B16 | 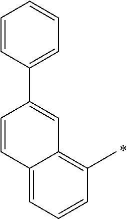 [SMILES: Cc1cccc2ccc(-c3ccccc3)cc12] | 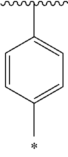 [SMILES: *c1ccc(C)cc1] | 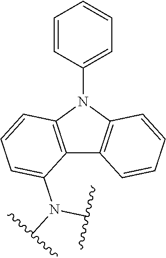 [SMILES: *N(*)c1cccc2c1c1ccccc1n2-c1ccccc1] | 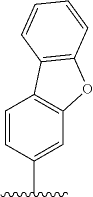 [SMILES: *c1ccc2c(c1)oc1ccccc12] |
| B17 | 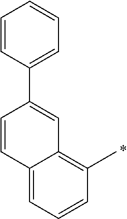 [SMILES: Cc1cccc2ccc(-c3ccccc3)cc12] | 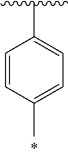 [SMILES: *c1ccc(C)cc1] | 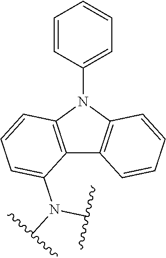 [SMILES: *N(*)c1cccc2c1c1ccccc1n2-c1ccccc1] | 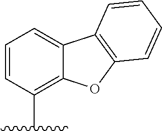 [SMILES: *c1cccc2c1oc1ccccc12] |
| B18 | 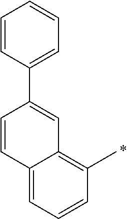 [SMILES: Cc1cccc2ccc(-c3ccccc3)cc12] | 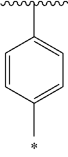 [SMILES: *c1ccc(C)cc1] | 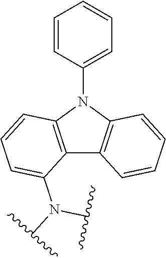 [SMILES: *N(*)c1cccc2c1c1ccccc1n2-c1ccccc1] | 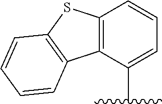 [SMILES: *c1cccc2sc3ccccc3c12] |
| B19 | 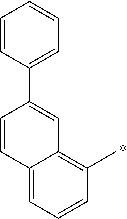 [SMILES: Cc1cccc2ccc(-c3ccccc3)cc12] | 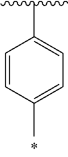 [SMILES: *c1ccc(C)cc1] | 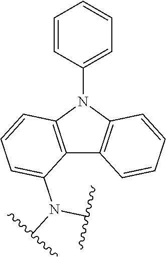 [SMILES: *N(*)c1cccc2c1c1ccccc1n2-c1ccccc1] | 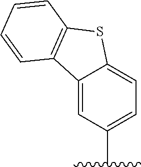 [SMILES: *c1ccc2sc3ccccc3c2c1] |
| B20 | 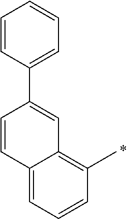 [SMILES: Cc1cccc2ccc(-c3ccccc3)cc12] | 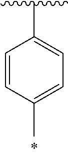 [SMILES: *c1ccc(C)cc1] | 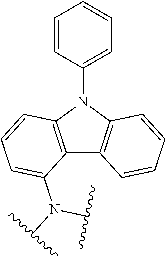 [SMILES: *N(*)c1cccc2c1c1ccccc1n2-c1ccccc1] | 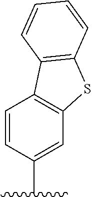 [SMILES: *c1ccc2c(c1)sc1ccccc12] |
| B21 | 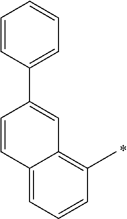 [SMILES: Cc1cccc2ccc(-c3ccccc3)cc12] | 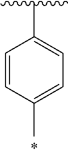 [SMILES: *c1ccc(C)cc1] | 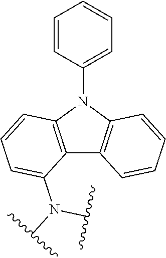 [SMILES: *N(*)c1cccc2c1c1ccccc1n2-c1ccccc1] | 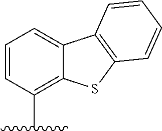 [SMILES: *c1cccc2c1sc1ccccc12] |
| B22 | 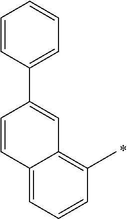 [SMILES: Cc1cccc2ccc(-c3ccccc3)cc12] | 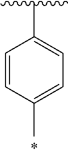 [SMILES: *c1ccc(C)cc1] | 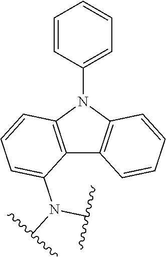 [SMILES: *N(*)c1cccc2c1c1ccccc1n2-c1ccccc1] | 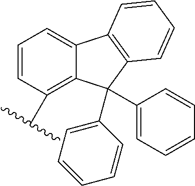 [SMILES: *c1cccc2c1C(c1ccccc1)(c1ccccc1)c1ccccc1-2] |
| B23 | 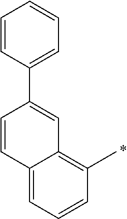 [SMILES: Cc1cccc2ccc(-c3ccccc3)cc12] | 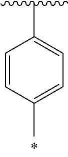 [SMILES: *c1ccc(C)cc1] | 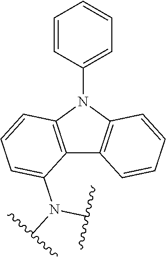 [SMILES: *N(*)c1cccc2c1c1ccccc1n2-c1ccccc1] | 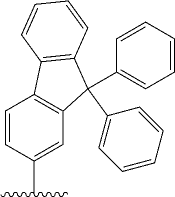 [SMILES: *c1ccc2c(c1)C(c1ccccc1)(c1ccccc1)c1ccccc1-2] |
| B24 | 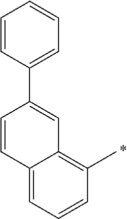 [SMILES: Cc1cccc2ccc(-c3ccccc3)cc12] | 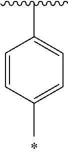 [SMILES: *c1ccc(C)cc1] | 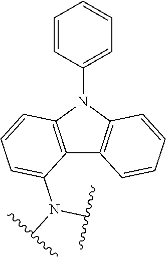 [SMILES: *N(*)c1cccc2c1c1ccccc1n2-c1ccccc1] | 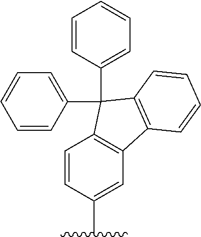 [SMILES: *c1ccc2c(c1)-c1ccccc1C2(c1ccccc1)c1ccccc1] |
| B25 | 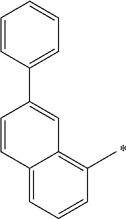 [SMILES: Cc1cccc2ccc(-c3ccccc3)cc12] | 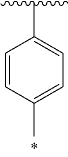 [SMILES: *c1ccc(C)cc1] | 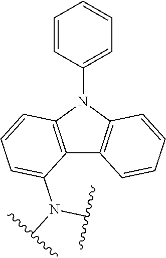 [SMILES: *N(*)c1cccc2c1c1ccccc1n2-c1ccccc1] | 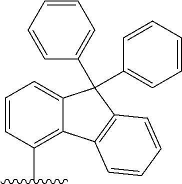 [SMILES: *c1cccc2c1-c1ccccc1C2(c1ccccc1)c1ccccc1] |
| B26 | 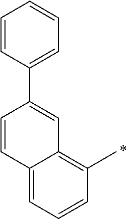 [SMILES: Cc1cccc2ccc(-c3ccccc3)cc12] | 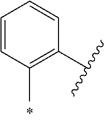 [SMILES: *c1ccccc1C] | 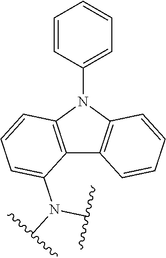 [SMILES: *N(*)c1cccc2c1c1ccccc1n2-c1ccccc1] | 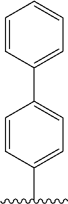 [SMILES: *c1ccc(-c2ccccc2)cc1] |
| B27 | 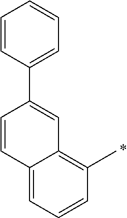 [SMILES: Cc1cccc2ccc(-c3ccccc3)cc12] | 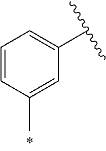 [SMILES: *c1cccc(C)c1] | 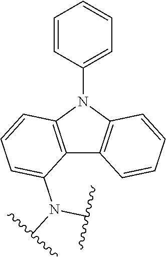 [SMILES: *N(*)c1cccc2c1c1ccccc1n2-c1ccccc1] | 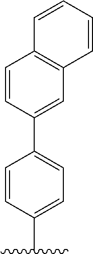 [SMILES: *c1ccc(-c2ccc3ccccc3c2)cc1] |
| B28 | 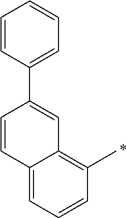 [SMILES: Cc1cccc2ccc(-c3ccccc3)cc12] | 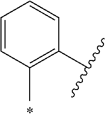 [SMILES: *c1ccccc1C] | 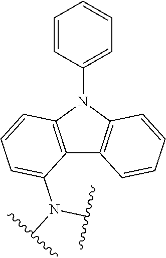 [SMILES: *N(*)c1cccc2c1c1ccccc1n2-c1ccccc1] | 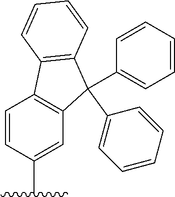 [SMILES: *c1ccc2c(c1)C(c1ccccc1)(c1ccccc1)c1ccccc1-2] |
| B29 | 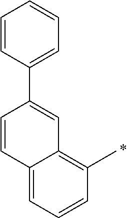 [SMILES: Cc1cccc2ccc(-c3ccccc3)cc12] | 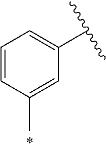 [SMILES: *c1cccc(C)c1] | 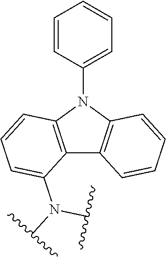 [SMILES: *N(*)c1cccc2c1c1ccccc1n2-c1ccccc1] | 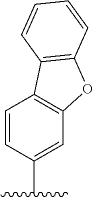 [SMILES: *c1ccc2c(c1)oc1ccccc12] |
| B30 | 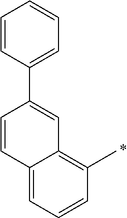 [SMILES: Cc1cccc2ccc(-c3ccccc3)cc12] | 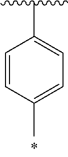 [SMILES: *c1ccc(C)cc1] | 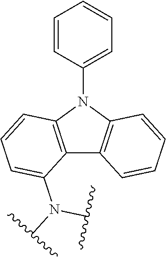 [SMILES: *N(*)c1cccc2c1c1ccccc1n2-c1ccccc1] | 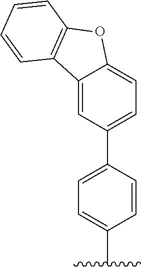 [SMILES: *c1ccc(-c2ccc3oc4ccccc4c3c2)cc1] |
| B31 | 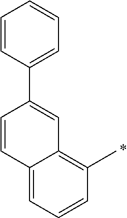 [SMILES: Cc1cccc2ccc(-c3ccccc3)cc12] | 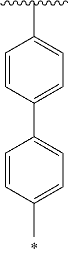 [SMILES: *c1ccc(-c2ccc(C)cc2)cc1] | 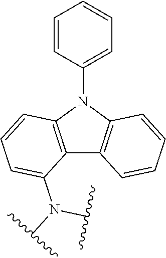 [SMILES: *N(*)c1cccc2c1c1ccccc1n2-c1ccccc1] | 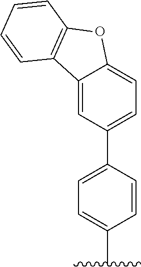 [SMILES: *c1ccc(-c2ccc3oc4ccccc4c3c2)cc1] |
| B32 | 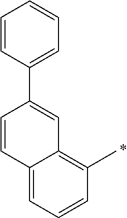 [SMILES: Cc1cccc2ccc(-c3ccccc3)cc12] | 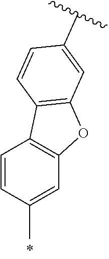 [SMILES: *c1ccc2c(c1)oc1cc(C)ccc12] | 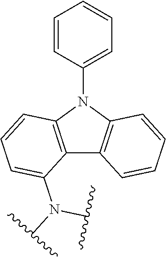 [SMILES: *N(*)c1cccc2c1c1ccccc1n2-c1ccccc1] | 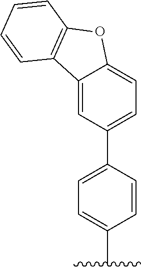 [SMILES: *c1ccc(-c2ccc3oc4ccccc4c3c2)cc1] |
| B33 | 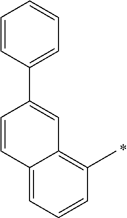 [SMILES: Cc1cccc2ccc(-c3ccccc3)cc12] | 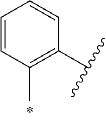 [SMILES: *c1ccccc1C] | 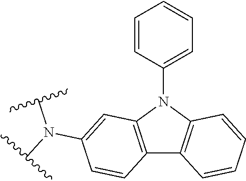 [SMILES: *N(*)c1ccc2c3ccccc3n(-c3ccccc3)c2c1] | 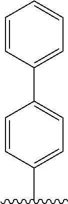 [SMILES: *c1ccc(-c2ccccc2)cc1] |
| B34 | 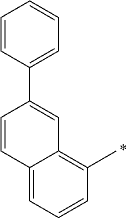 [SMILES: Cc1cccc2ccc(-c3ccccc3)cc12] | 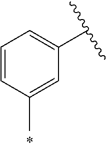 [SMILES: *c1cccc(C)c1] | 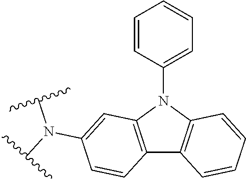 [SMILES: *N(*)c1ccc2c3ccccc3n(-c3ccccc3)c2c1] | 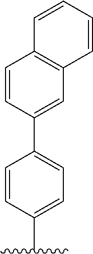 [SMILES: *c1ccc(-c2ccc3ccccc3c2)cc1] |
| B35 | 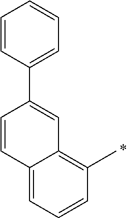 [SMILES: Cc1cccc2ccc(-c3ccccc3)cc12] | 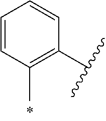 [SMILES: *c1ccccc1C] | 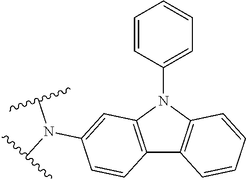 [SMILES: *N(*)c1ccc2c3ccccc3n(-c3ccccc3)c2c1] | 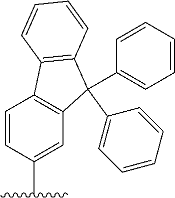 [SMILES: *c1ccc2c(c1)C(c1ccccc1)(c1ccccc1)c1ccccc1-2] |
| B36 | 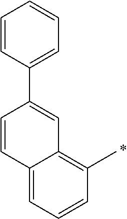 [SMILES: Cc1cccc2ccc(-c3ccccc3)cc12] | 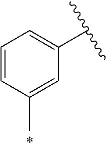 [SMILES: *c1cccc(C)c1] | 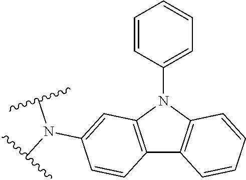 [SMILES: *N(*)c1ccc2c3ccccc3n(-c3ccccc3)c2c1] | 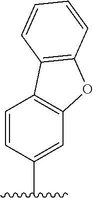 [SMILES: *c1ccc2c(c1)oc1ccccc12] |
| B37 | 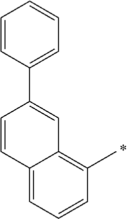 [SMILES: Cc1cccc2ccc(-c3ccccc3)cc12] | 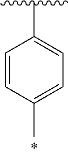 [SMILES: *c1ccc(C)cc1] | 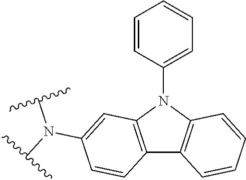 [SMILES: *N(*)c1ccc2c3ccccc3n(-c3ccccc3)c2c1] | 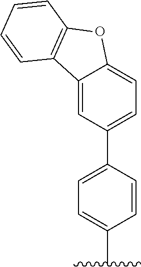 [SMILES: *c1ccc(-c2ccc3oc4ccccc4c3c2)cc1] |
| B38 | 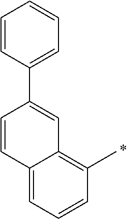 [SMILES: Cc1cccc2ccc(-c3ccccc3)cc12] | 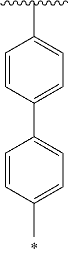 [SMILES: *c1ccc(-c2ccc(C)cc2)cc1] | 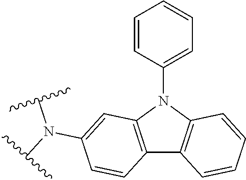 [SMILES: *N(*)c1ccc2c3ccccc3n(-c3ccccc3)c2c1] | 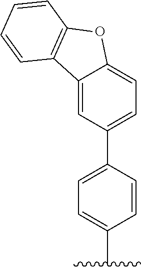 [SMILES: *c1ccc(-c2ccc3oc4ccccc4c3c2)cc1] |
| B39 | 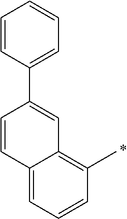 [SMILES: Cc1cccc2ccc(-c3ccccc3)cc12] | 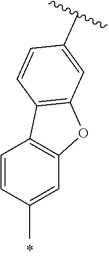 [SMILES: *c1ccc2c(c1)oc1cc(C)ccc12] | 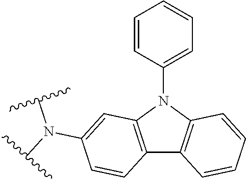 [SMILES: *N(*)c1ccc2c3ccccc3n(-c3ccccc3)c2c1] | 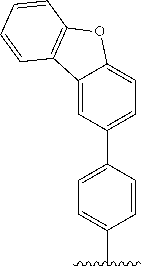 [SMILES: *c1ccc(-c2ccc3oc4ccccc4c3c2)cc1] |
| C1 | 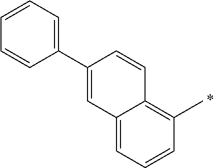 [SMILES: Cc1cccc2cc(-c3ccccc3)ccc12] | 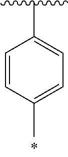 [SMILES: *c1ccc(C)cc1] | 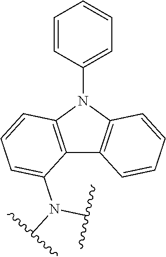 [SMILES: *N(*)c1cccc2c1c1ccccc1n2-c1ccccc1] | 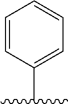 [SMILES: *c1ccccc1] |
| C2 | 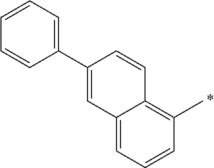 [SMILES: Cc1cccc2cc(-c3ccccc3)ccc12] | 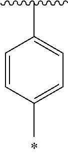 [SMILES: *c1ccc(C)cc1] | 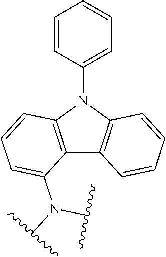 [SMILES: *N(*)c1cccc2c1c1ccccc1n2-c1ccccc1] | 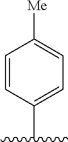 [SMILES: *c1ccc(C)cc1] |
| C3 | 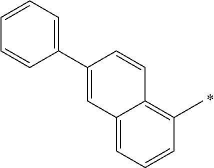 [SMILES: Cc1cccc2cc(-c3ccccc3)ccc12] | 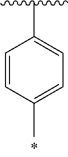 [SMILES: *c1ccc(C)cc1] | 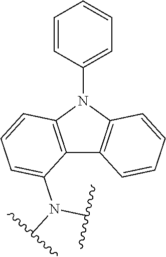 [SMILES: *N(*)c1cccc2c1c1ccccc1n2-c1ccccc1] | 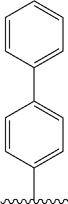 [SMILES: *c1ccc(-c2ccccc2)cc1] |
| C4 | 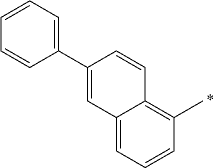 [SMILES: Cc1cccc2cc(-c3ccccc3)ccc12] | 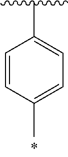 [SMILES: *c1ccc(C)cc1] | 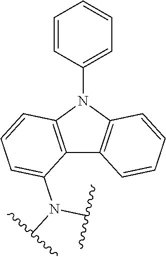 [SMILES: *N(*)c1cccc2c1c1ccccc1n2-c1ccccc1] | 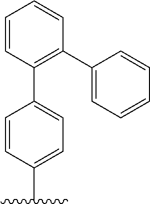 [SMILES: *c1ccc(-c2ccccc2-c2ccccc2)cc1] |
| C5 | 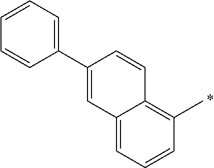 [SMILES: Cc1cccc2cc(-c3ccccc3)ccc12] | 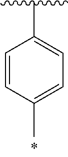 [SMILES: *c1ccc(C)cc1] | 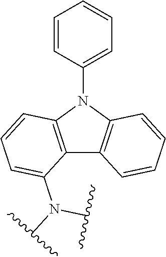 [SMILES: *N(*)c1cccc2c1c1ccccc1n2-c1ccccc1] | 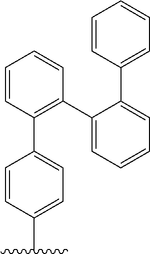 [SMILES: *c1ccc(-c2ccccc2-c2ccccc2-c2ccccc2)cc1] |
| C6 | 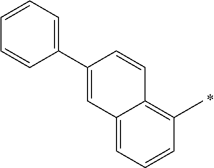 [SMILES: Cc1cccc2cc(-c3ccccc3)ccc12] | 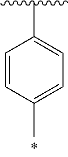 [SMILES: *c1ccc(C)cc1] | 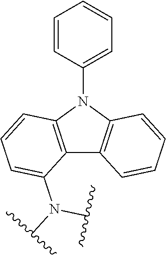 [SMILES: *N(*)c1cccc2c1c1ccccc1n2-c1ccccc1] | 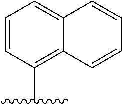 [SMILES: *c1cccc2ccccc12] |
| C7 | 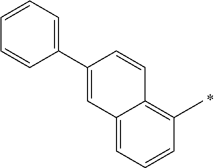 [SMILES: Cc1cccc2cc(-c3ccccc3)ccc12] | 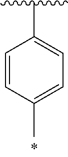 [SMILES: *c1ccc(C)cc1] | 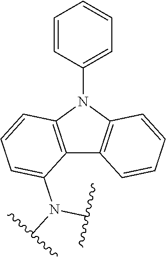 [SMILES: *N(*)c1cccc2c1c1ccccc1n2-c1ccccc1] | 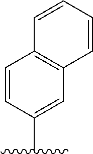 [SMILES: *c1ccc2ccccc2c1] |
| C8 | 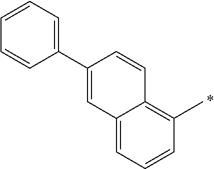 [SMILES: Cc1cccc2cc(-c3ccccc3)ccc12] | 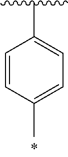 [SMILES: *c1ccc(C)cc1] | 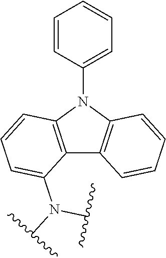 [SMILES: *N(*)c1cccc2c1c1ccccc1n2-c1ccccc1] | 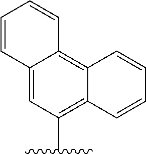 [SMILES: *c1cc2ccccc2c2ccccc12] |
| C9 | 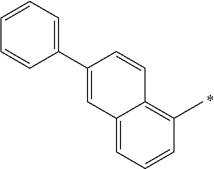 [SMILES: Cc1cccc2cc(-c3ccccc3)ccc12] | 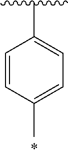 [SMILES: *c1ccc(C)cc1] | 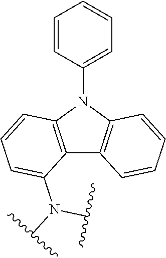 [SMILES: *N(*)c1cccc2c1c1ccccc1n2-c1ccccc1] | 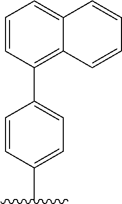 [SMILES: *c1ccc(-c2cccc3ccccc23)cc1] |
| C10 | 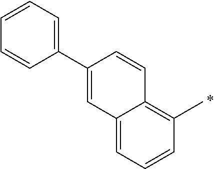 [SMILES: Cc1cccc2cc(-c3ccccc3)ccc12] | 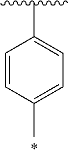 [SMILES: *c1ccc(C)cc1] | 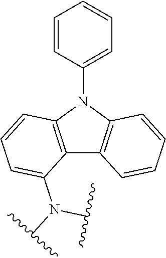 [SMILES: *N(*)c1cccc2c1c1ccccc1n2-c1ccccc1] | 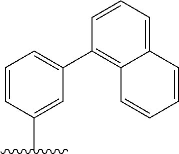 [SMILES: *c1cccc(-c2cccc3ccccc23)c1] |
| C11 | 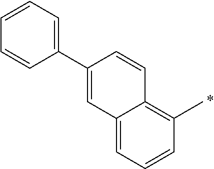 [SMILES: Cc1cccc2cc(-c3ccccc3)ccc12] | 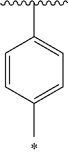 [SMILES: *c1ccc(C)cc1] | 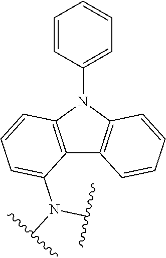 [SMILES: *N(*)c1cccc2c1c1ccccc1n2-c1ccccc1] | 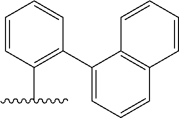 [SMILES: *c1ccccc1-c1cccc2ccccc12] |
| C12 | 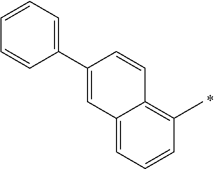 [SMILES: Cc1cccc2cc(-c3ccccc3)ccc12] | 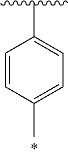 [SMILES: *c1ccc(C)cc1] | 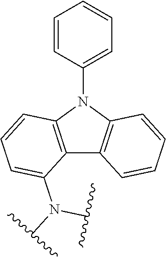 [SMILES: *N(*)c1cccc2c1c1ccccc1n2-c1ccccc1] | 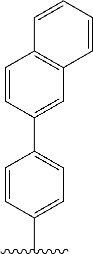 [SMILES: *c1ccc(-c2ccc3ccccc3c2)cc1] |
| C13 | 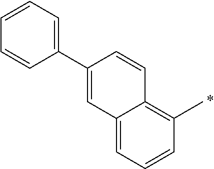 [SMILES: Cc1cccc2cc(-c3ccccc3)ccc12] | 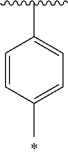 [SMILES: *c1ccc(C)cc1] | 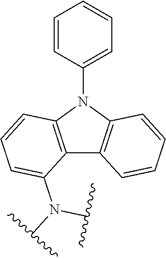 [SMILES: *N(*)c1cccc2c1c1ccccc1n2-c1ccccc1] | 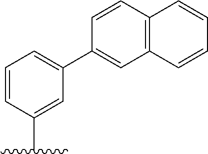 [SMILES: *c1cccc(-c2ccc3ccccc3c2)c1] |
| C14 | 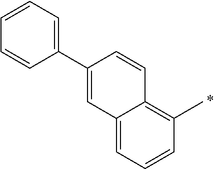 [SMILES: Cc1cccc2cc(-c3ccccc3)ccc12] | 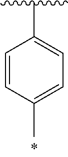 [SMILES: *c1ccc(C)cc1] | 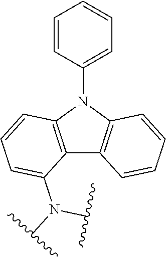 [SMILES: *N(*)c1cccc2c1c1ccccc1n2-c1ccccc1] | 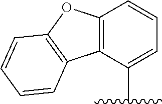 [SMILES: *c1cccc2oc3ccccc3c12] |
| C15 | 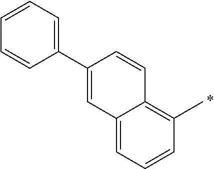 [SMILES: Cc1cccc2cc(-c3ccccc3)ccc12] | 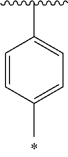 [SMILES: *c1ccc(C)cc1] | 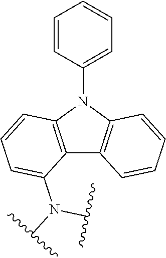 [SMILES: *N(*)c1cccc2c1c1ccccc1n2-c1ccccc1] | 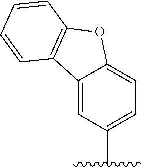 [SMILES: *c1ccc2oc3ccccc3c2c1] |
| C16 | 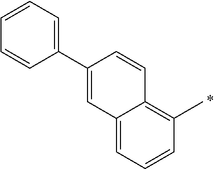 [SMILES: Cc1cccc2cc(-c3ccccc3)ccc12] | 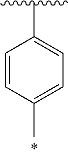 [SMILES: *c1ccc(C)cc1] | 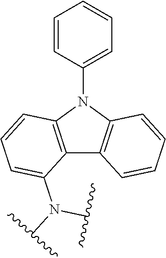 [SMILES: *N(*)c1cccc2c1c1ccccc1n2-c1ccccc1] | 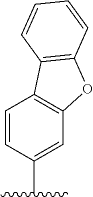 [SMILES: *c1ccc2c(c1)oc1ccccc12] |
| C17 | 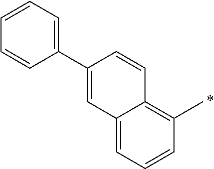 [SMILES: Cc1cccc2cc(-c3ccccc3)ccc12] | 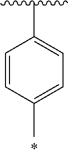 [SMILES: *c1ccc(C)cc1] | 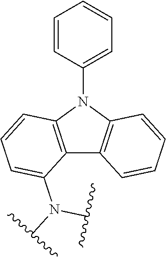 [SMILES: *N(*)c1cccc2c1c1ccccc1n2-c1ccccc1] | 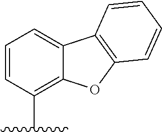 [SMILES: *c1cccc2c1oc1ccccc12] |
| C18 | 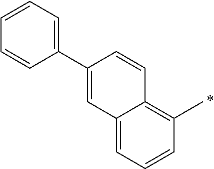 [SMILES: Cc1cccc2cc(-c3ccccc3)ccc12] | 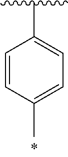 [SMILES: *c1ccc(C)cc1] | 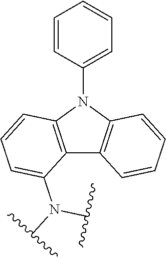 [SMILES: *N(*)c1cccc2c1c1ccccc1n2-c1ccccc1] | 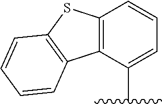 [SMILES: *c1cccc2sc3ccccc3c12] |
| C19 | 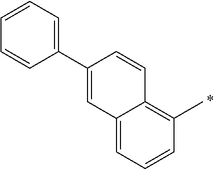 [SMILES: Cc1cccc2cc(-c3ccccc3)ccc12] | 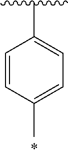 [SMILES: *c1ccc(C)cc1] | 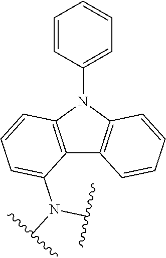 [SMILES: *N(*)c1cccc2c1c1ccccc1n2-c1ccccc1] | 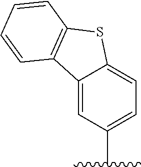 [SMILES: *c1ccc2sc3ccccc3c2c1] |
| C20 | 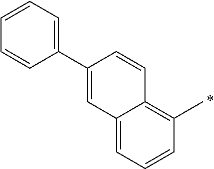 [SMILES: Cc1cccc2cc(-c3ccccc3)ccc12] | 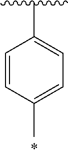 [SMILES: *c1ccc(C)cc1] | 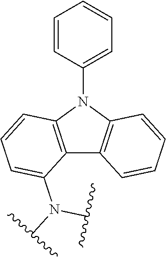 [SMILES: *N(*)c1cccc2c1c1ccccc1n2-c1ccccc1] | 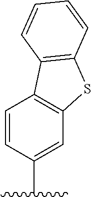 [SMILES: *c1ccc2c(c1)sc1ccccc12] |
| C21 | 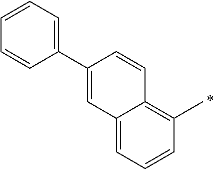 [SMILES: Cc1cccc2cc(-c3ccccc3)ccc12] | 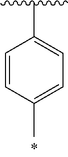 [SMILES: *c1ccc(C)cc1] | 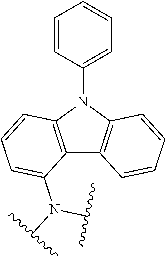 [SMILES: *N(*)c1cccc2c1c1ccccc1n2-c1ccccc1] | 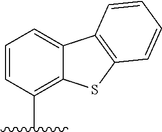 [SMILES: *c1cccc2c1sc1ccccc12] |
| C22 | 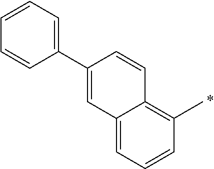 [SMILES: Cc1cccc2cc(-c3ccccc3)ccc12] | 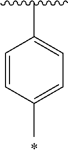 [SMILES: *c1ccc(C)cc1] | 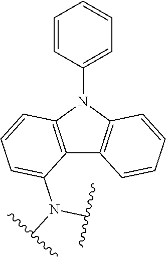 [SMILES: *N(*)c1cccc2c1c1ccccc1n2-c1ccccc1] | 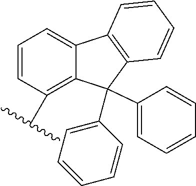 [SMILES: *c1cccc2c1C(c1ccccc1)(c1ccccc1)c1ccccc1-2] |
| C23 | 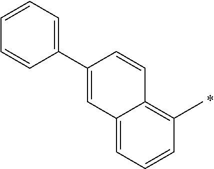 [SMILES: Cc1cccc2cc(-c3ccccc3)ccc12] | 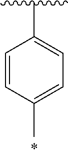 [SMILES: *c1ccc(C)cc1] | 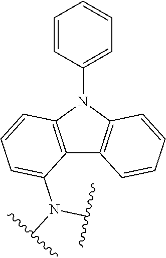 [SMILES: *N(*)c1cccc2c1c1ccccc1n2-c1ccccc1] | 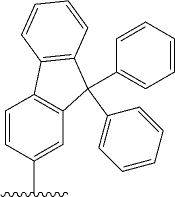 [SMILES: *c1ccc2c(c1)C(c1ccccc1)(c1ccccc1)c1ccccc1-2] |
| C24 | 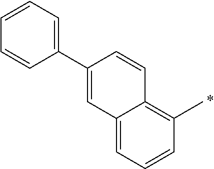 [SMILES: Cc1cccc2cc(-c3ccccc3)ccc12] | 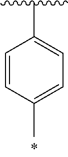 [SMILES: *c1ccc(C)cc1] | 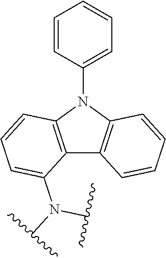 [SMILES: *N(*)c1cccc2c1c1ccccc1n2-c1ccccc1] | 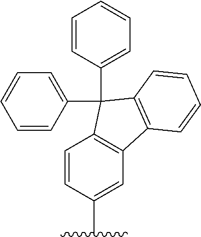 [SMILES: *c1ccc2c(c1)-c1ccccc1C2(c1ccccc1)c1ccccc1] |
| C25 | 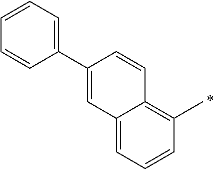 [SMILES: Cc1cccc2cc(-c3ccccc3)ccc12] | 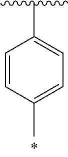 [SMILES: *c1ccc(C)cc1] | 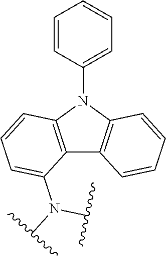 [SMILES: *N(*)c1cccc2c1c1ccccc1n2-c1ccccc1] | 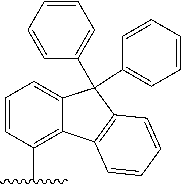 [SMILES: *c1cccc2c1-c1ccccc1C2(c1ccccc1)c1ccccc1] |
| C26 | 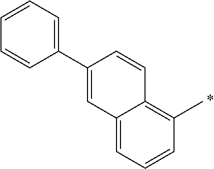 [SMILES: Cc1cccc2cc(-c3ccccc3)ccc12] | 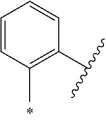 [SMILES: *c1ccccc1C] | 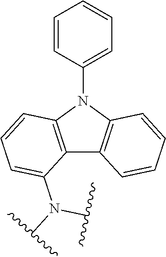 [SMILES: *N(*)c1cccc2c1c1ccccc1n2-c1ccccc1] | 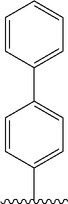 [SMILES: *c1ccc(-c2ccccc2)cc1] |
| C27 | 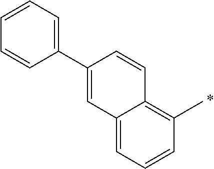 [SMILES: Cc1cccc2cc(-c3ccccc3)ccc12] | 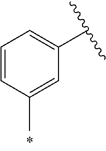 [SMILES: *c1cccc(C)c1] | 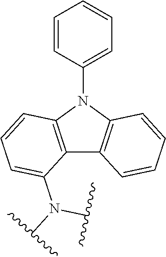 [SMILES: *N(*)c1cccc2c1c1ccccc1n2-c1ccccc1] | 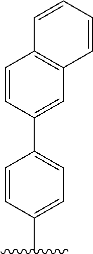 [SMILES: *c1ccc(-c2ccc3ccccc3c2)cc1] |
| C28 | 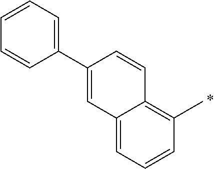 [SMILES: Cc1cccc2cc(-c3ccccc3)ccc12] | 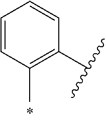 [SMILES: *c1ccccc1C] | 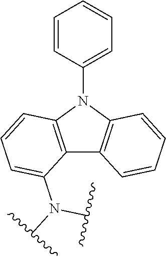 [SMILES: *N(*)c1cccc2c1c1ccccc1n2-c1ccccc1] | 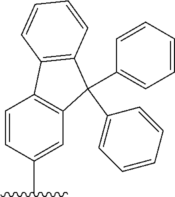 [SMILES: *c1ccc2c(c1)C(c1ccccc1)(c1ccccc1)c1ccccc1-2] |
| C29 | 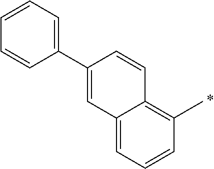 [SMILES: Cc1cccc2cc(-c3ccccc3)ccc12] | 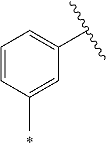 [SMILES: *c1cccc(C)c1] | 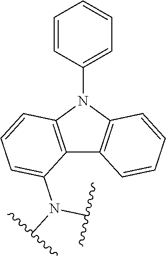 [SMILES: *N(*)c1cccc2c1c1ccccc1n2-c1ccccc1] | 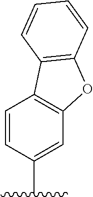 [SMILES: *c1ccc2c(c1)oc1ccccc12] |
| C30 | 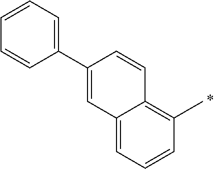 [SMILES: Cc1cccc2cc(-c3ccccc3)ccc12] | 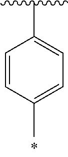 [SMILES: *c1ccc(C)cc1] | 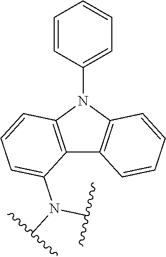 [SMILES: *N(*)c1cccc2c1c1ccccc1n2-c1ccccc1] | 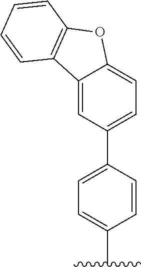 [SMILES: *c1ccc(-c2ccc3oc4ccccc4c3c2)cc1] |
| C31 | 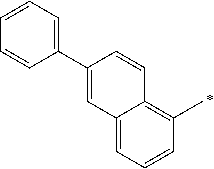 [SMILES: Cc1cccc2cc(-c3ccccc3)ccc12] | 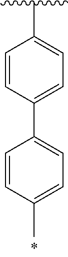 [SMILES: *c1ccc(-c2ccc(C)cc2)cc1] | 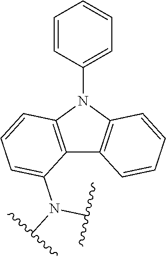 [SMILES: *N(*)c1cccc2c1c1ccccc1n2-c1ccccc1] | 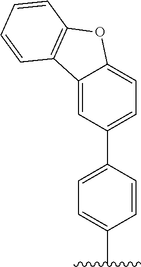 [SMILES: *c1ccc(-c2ccc3oc4ccccc4c3c2)cc1] |
| C32 | 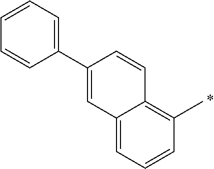 [SMILES: Cc1cccc2cc(-c3ccccc3)ccc12] | 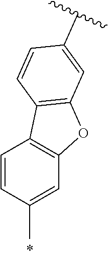 [SMILES: *c1ccc2c(c1)oc1cc(C)ccc12] | 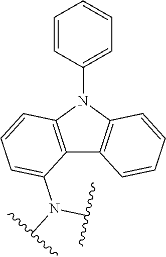 [SMILES: *N(*)c1cccc2c1c1ccccc1n2-c1ccccc1] | 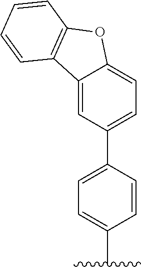 [SMILES: *c1ccc(-c2ccc3oc4ccccc4c3c2)cc1] |
| C33 | 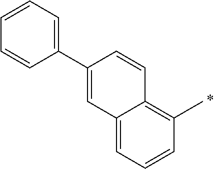 [SMILES: Cc1cccc2cc(-c3ccccc3)ccc12] | 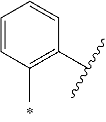 [SMILES: *c1ccccc1C] | 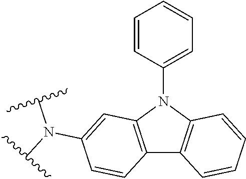 [SMILES: *N(*)c1ccc2c3ccccc3n(-c3ccccc3)c2c1] | 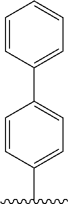 [SMILES: *c1ccc(-c2ccccc2)cc1] |
| C34 | 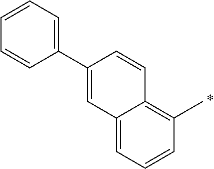 [SMILES: Cc1cccc2cc(-c3ccccc3)ccc12] | 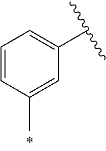 [SMILES: *c1cccc(C)c1] | 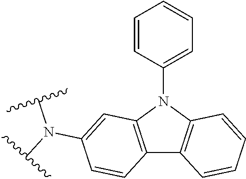 [SMILES: *N(*)c1ccc2c3ccccc3n(-c3ccccc3)c2c1] | 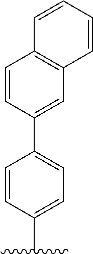 [SMILES: *c1ccc(-c2ccc3ccccc3c2)cc1] |
| C35 | 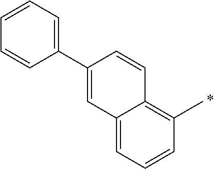 [SMILES: Cc1cccc2cc(-c3ccccc3)ccc12] | 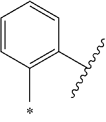 [SMILES: *c1ccccc1C] | 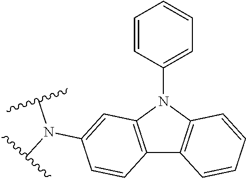 [SMILES: *N(*)c1ccc2c3ccccc3n(-c3ccccc3)c2c1] | 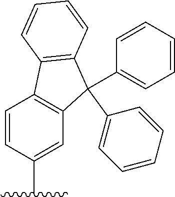 [SMILES: *c1ccc2c(c1)C(c1ccccc1)(c1ccccc1)c1ccccc1-2] |
| C36 | 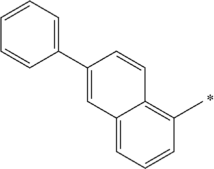 [SMILES: Cc1cccc2cc(-c3ccccc3)ccc12] | 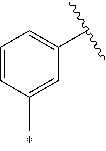 [SMILES: *c1cccc(C)c1] | 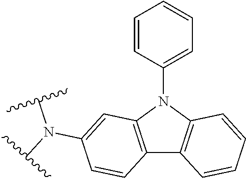 [SMILES: *N(*)c1ccc2c3ccccc3n(-c3ccccc3)c2c1] | 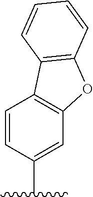 [SMILES: *c1ccc2c(c1)oc1ccccc12] |
| C37 | 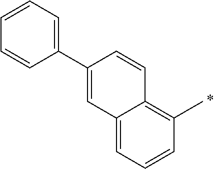 [SMILES: Cc1cccc2cc(-c3ccccc3)ccc12] | 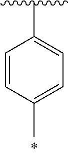 [SMILES: *c1ccc(C)cc1] | 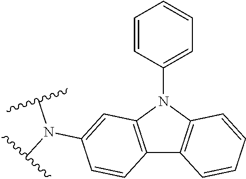 [SMILES: *N(*)c1ccc2c3ccccc3n(-c3ccccc3)c2c1] | 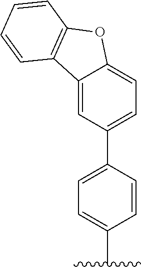 [SMILES: *c1ccc(-c2ccc3oc4ccccc4c3c2)cc1] |
| C38 | 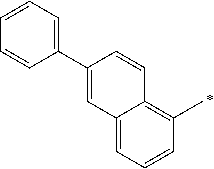 [SMILES: Cc1cccc2cc(-c3ccccc3)ccc12] | 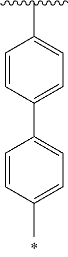 [SMILES: *c1ccc(-c2ccc(C)cc2)cc1] | 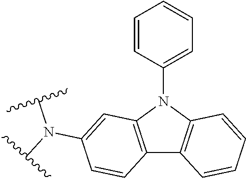 [SMILES: *N(*)c1ccc2c3ccccc3n(-c3ccccc3)c2c1] | 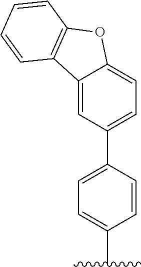 [SMILES: *c1ccc(-c2ccc3oc4ccccc4c3c2)cc1] |
| C39 | 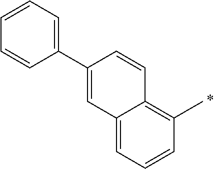 [SMILES: Cc1cccc2cc(-c3ccccc3)ccc12] | 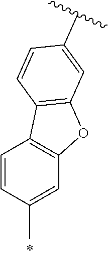 [SMILES: *c1ccc2c(c1)oc1cc(C)ccc12] | 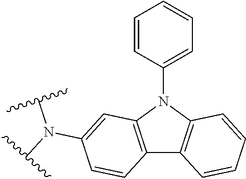 [SMILES: *N(*)c1ccc2c3ccccc3n(-c3ccccc3)c2c1] | 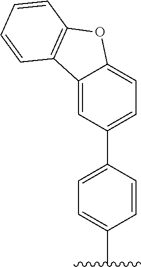 [SMILES: *c1ccc(-c2ccc3oc4ccccc4c3c2)cc1] |
| D1 | 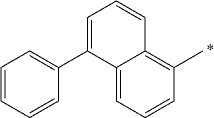 [SMILES: Cc1cccc2c(-c3ccccc3)cccc12] | 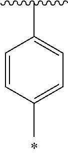 [SMILES: *c1ccc(C)cc1] | 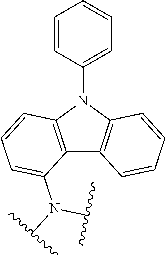 [SMILES: *N(*)c1cccc2c1c1ccccc1n2-c1ccccc1] | 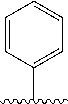 [SMILES: *c1ccccc1] |
| D2 | 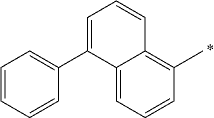 [SMILES: Cc1cccc2c(-c3ccccc3)cccc12] | 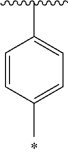 [SMILES: *c1ccc(C)cc1] | 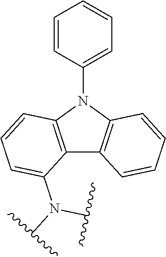 [SMILES: *N(*)c1cccc2c1c1ccccc1n2-c1ccccc1] | 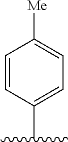 [SMILES: *c1ccc(C)cc1] |
| D3 | 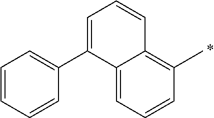 [SMILES: Cc1cccc2c(-c3ccccc3)cccc12] | 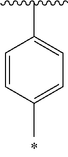 [SMILES: *c1ccc(C)cc1] | 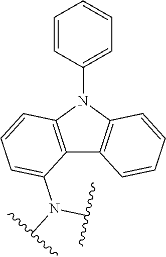 [SMILES: *N(*)c1cccc2c1c1ccccc1n2-c1ccccc1] | 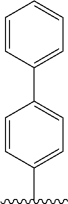 [SMILES: *c1ccc(-c2ccccc2)cc1] |
| D4 | 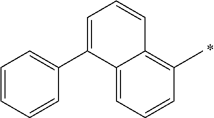 [SMILES: Cc1cccc2c(-c3ccccc3)cccc12] | 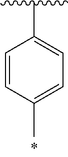 [SMILES: *c1ccc(C)cc1] | 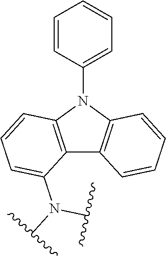 [SMILES: *N(*)c1cccc2c1c1ccccc1n2-c1ccccc1] | 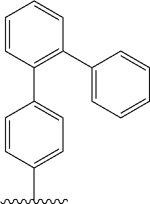 [SMILES: *c1ccc(-c2ccccc2-c2ccccc2)cc1] |
| D5 | 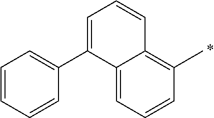 [SMILES: Cc1cccc2c(-c3ccccc3)cccc12] | 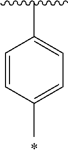 [SMILES: *c1ccc(C)cc1] | 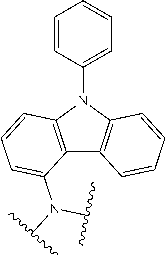 [SMILES: *N(*)c1cccc2c1c1ccccc1n2-c1ccccc1] | 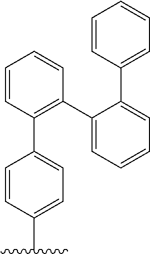 [SMILES: *c1ccc(-c2ccccc2-c2ccccc2-c2ccccc2)cc1] |
| D6 | 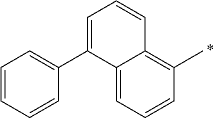 [SMILES: Cc1cccc2c(-c3ccccc3)cccc12] | 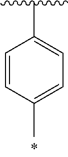 [SMILES: *c1ccc(C)cc1] | 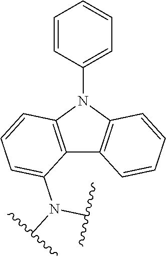 [SMILES: *N(*)c1cccc2c1c1ccccc1n2-c1ccccc1] | 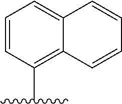 [SMILES: *c1cccc2ccccc12] |
| D7 | 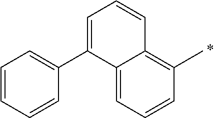 [SMILES: Cc1cccc2c(-c3ccccc3)cccc12] | 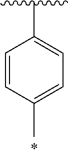 [SMILES: *c1ccc(C)cc1] | 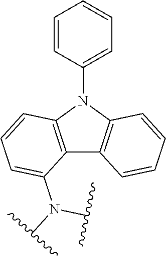 [SMILES: *N(*)c1cccc2c1c1ccccc1n2-c1ccccc1] | 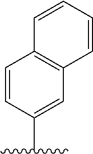 [SMILES: *c1ccc2ccccc2c1] |
| D8 | 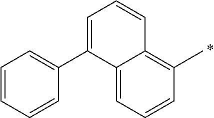 [SMILES: Cc1cccc2c(-c3ccccc3)cccc12] | 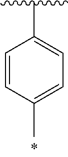 [SMILES: *c1ccc(C)cc1] | 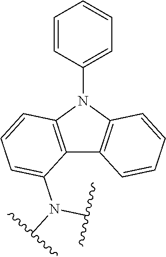 [SMILES: *N(*)c1cccc2c1c1ccccc1n2-c1ccccc1] | 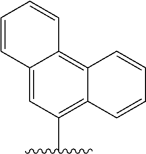 [SMILES: *c1cc2ccccc2c2ccccc12] |
| D9 | 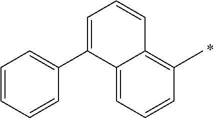 [SMILES: Cc1cccc2c(-c3ccccc3)cccc12] | 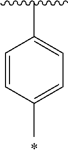 [SMILES: *c1ccc(C)cc1] | 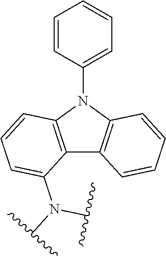 [SMILES: *N(*)c1cccc2c1c1ccccc1n2-c1ccccc1] | 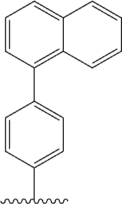 [SMILES: *c1ccc(-c2cccc3ccccc23)cc1] |
| D10 | 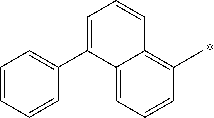 [SMILES: Cc1cccc2c(-c3ccccc3)cccc12] | 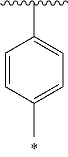 [SMILES: *c1ccc(C)cc1] | 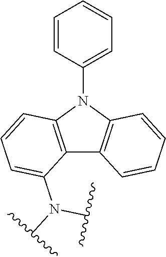 [SMILES: *N(*)c1cccc2c1c1ccccc1n2-c1ccccc1] | 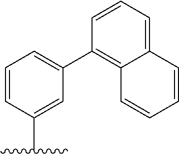 [SMILES: *c1cccc(-c2cccc3ccccc23)c1] |
| D11 | 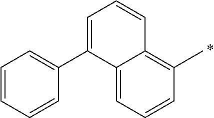 [SMILES: Cc1cccc2c(-c3ccccc3)cccc12] | 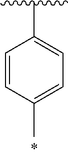 [SMILES: *c1ccc(C)cc1] | 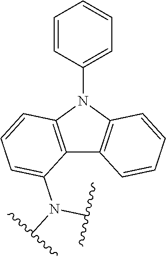 [SMILES: *N(*)c1cccc2c1c1ccccc1n2-c1ccccc1] | 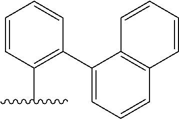 [SMILES: *c1ccccc1-c1cccc2ccccc12] |
| D12 | 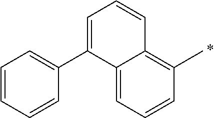 [SMILES: Cc1cccc2c(-c3ccccc3)cccc12] | 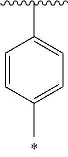 [SMILES: *c1ccc(C)cc1] | 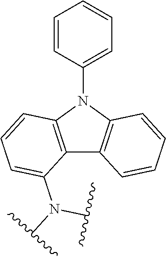 [SMILES: *N(*)c1cccc2c1c1ccccc1n2-c1ccccc1] | 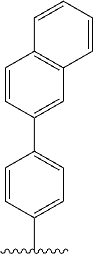 [SMILES: *c1ccc(-c2ccc3ccccc3c2)cc1] |
| D13 | 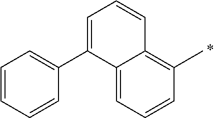 [SMILES: Cc1cccc2c(-c3ccccc3)cccc12] | 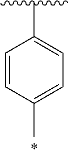 [SMILES: *c1ccc(C)cc1] | 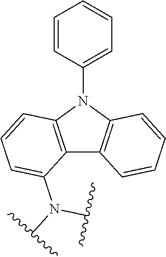 [SMILES: *N(*)c1cccc2c1c1ccccc1n2-c1ccccc1] | 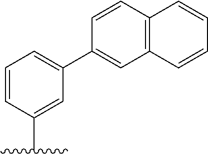 [SMILES: *c1cccc(-c2ccc3ccccc3c2)c1] |
| D14 | 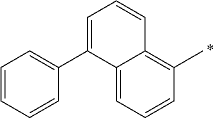 [SMILES: Cc1cccc2c(-c3ccccc3)cccc12] | 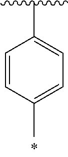 [SMILES: *c1ccc(C)cc1] | 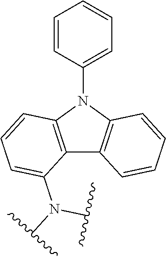 [SMILES: *N(*)c1cccc2c1c1ccccc1n2-c1ccccc1] | 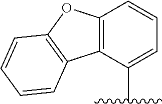 [SMILES: *c1cccc2oc3ccccc3c12] |
| D15 | 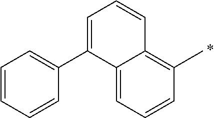 [SMILES: Cc1cccc2c(-c3ccccc3)cccc12] | 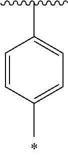 [SMILES: *c1ccc(C)cc1] | 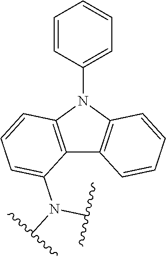 [SMILES: *N(*)c1cccc2c1c1ccccc1n2-c1ccccc1] | 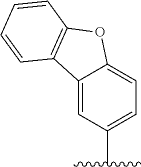 [SMILES: *c1ccc2oc3ccccc3c2c1] |
| D16 | 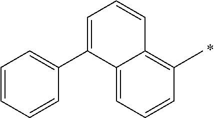 [SMILES: Cc1cccc2c(-c3ccccc3)cccc12] | 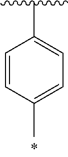 [SMILES: *c1ccc(C)cc1] | 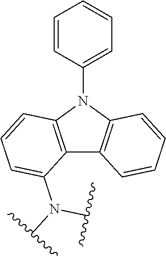 [SMILES: *N(*)c1cccc2c1c1ccccc1n2-c1ccccc1] | 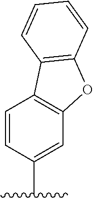 [SMILES: *c1ccc2c(c1)oc1ccccc12] |
| D17 | 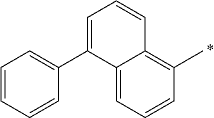 [SMILES: Cc1cccc2c(-c3ccccc3)cccc12] | 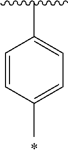 [SMILES: *c1ccc(C)cc1] | 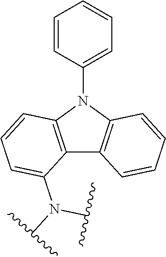 [SMILES: *N(*)c1cccc2c1c1ccccc1n2-c1ccccc1] | 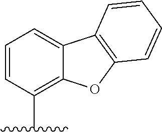 [SMILES: *c1cccc2c1oc1ccccc12] |
| D18 | 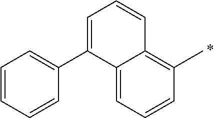 [SMILES: Cc1cccc2c(-c3ccccc3)cccc12] | 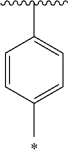 [SMILES: *c1ccc(C)cc1] | 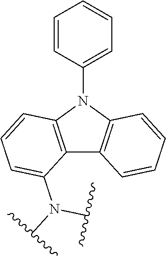 [SMILES: *N(*)c1cccc2c1c1ccccc1n2-c1ccccc1] | 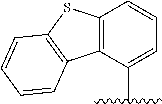 [SMILES: *c1cccc2sc3ccccc3c12] |
| D19 | 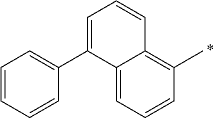 [SMILES: Cc1cccc2c(-c3ccccc3)cccc12] | 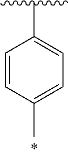 [SMILES: *c1ccc(C)cc1] | 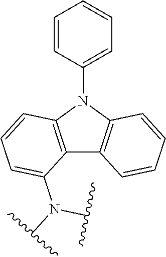 [SMILES: *N(*)c1cccc2c1c1ccccc1n2-c1ccccc1] | 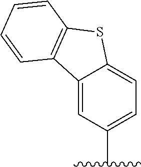 [SMILES: *c1ccc2sc3ccccc3c2c1] |
| D20 | 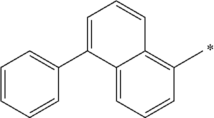 [SMILES: Cc1cccc2c(-c3ccccc3)cccc12] | 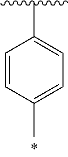 [SMILES: *c1ccc(C)cc1] | 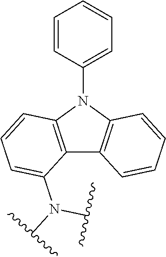 [SMILES: *N(*)c1cccc2c1c1ccccc1n2-c1ccccc1] | 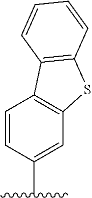 [SMILES: *c1ccc2c(c1)sc1ccccc12] |
| D21 | 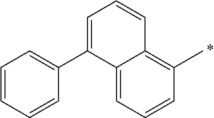 [SMILES: Cc1cccc2c(-c3ccccc3)cccc12] | 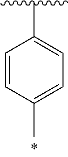 [SMILES: *c1ccc(C)cc1] | 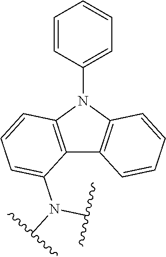 [SMILES: *N(*)c1cccc2c1c1ccccc1n2-c1ccccc1] | 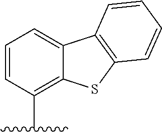 [SMILES: *c1cccc2c1sc1ccccc12] |
| D22 | 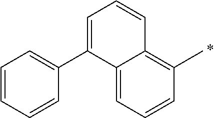 [SMILES: Cc1cccc2c(-c3ccccc3)cccc12] | 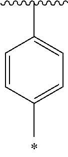 [SMILES: *c1ccc(C)cc1] | 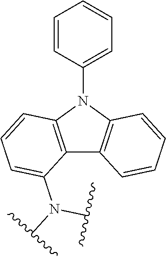 [SMILES: *N(*)c1cccc2c1c1ccccc1n2-c1ccccc1] | 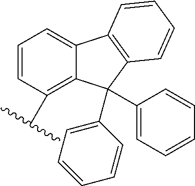 [SMILES: *c1cccc2c1C(c1ccccc1)(c1ccccc1)c1ccccc1-2] |
| D23 | 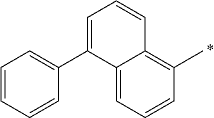 [SMILES: Cc1cccc2c(-c3ccccc3)cccc12] | 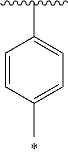 [SMILES: *c1ccc(C)cc1] | 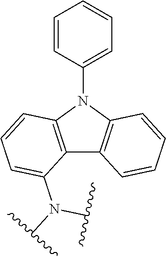 [SMILES: *N(*)c1cccc2c1c1ccccc1n2-c1ccccc1] | 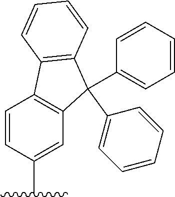 [SMILES: *c1ccc2c(c1)C(c1ccccc1)(c1ccccc1)c1ccccc1-2] |
| D24 | 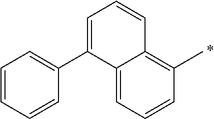 [SMILES: Cc1cccc2c(-c3ccccc3)cccc12] | 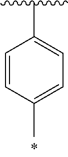 [SMILES: *c1ccc(C)cc1] | 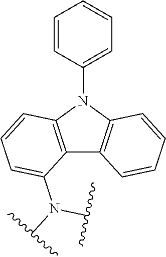 [SMILES: *N(*)c1cccc2c1c1ccccc1n2-c1ccccc1] | 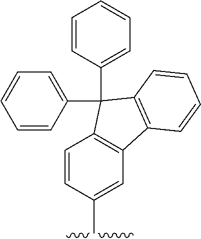 |
| D25 | 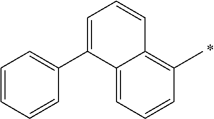 [SMILES: Cc1cccc2c(-c3ccccc3)cccc12] | 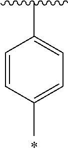 [SMILES: *c1ccc(C)cc1] | 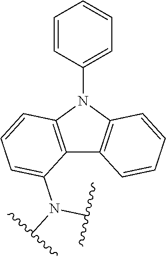 [SMILES: *N(*)c1cccc2c1c1ccccc1n2-c1ccccc1] | 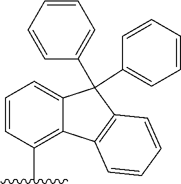 [SMILES: *c1cccc2c1-c1ccccc1C2(c1ccccc1)c1ccccc1] |
| D26 | 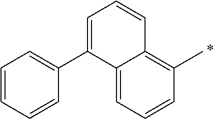 [SMILES: Cc1cccc2c(-c3ccccc3)cccc12] | 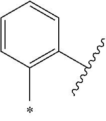 [SMILES: *c1ccccc1C] | 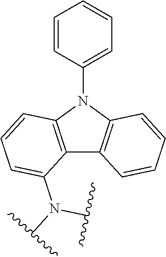 [SMILES: *N(*)c1cccc2c1c1ccccc1n2-c1ccccc1] | 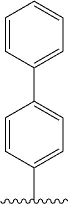 [SMILES: *c1ccc(-c2ccccc2)cc1] |
| D27 | 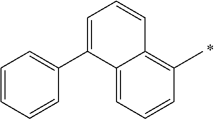 [SMILES: Cc1cccc2c(-c3ccccc3)cccc12] | 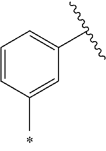 [SMILES: *c1cccc(C)c1] | 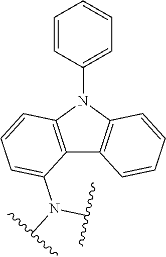 [SMILES: *N(*)c1cccc2c1c1ccccc1n2-c1ccccc1] | 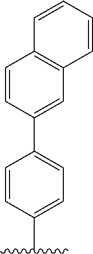 [SMILES: *c1ccc(-c2ccc3ccccc3c2)cc1] |
| D28 | 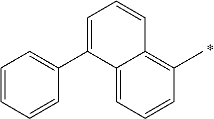 [SMILES: Cc1cccc2c(-c3ccccc3)cccc12] | 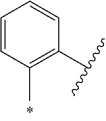 [SMILES: *c1ccccc1C] | 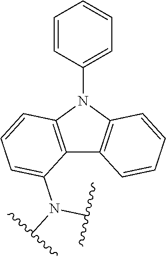 [SMILES: *N(*)c1cccc2c1c1ccccc1n2-c1ccccc1] | 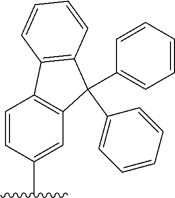 [SMILES: *c1ccc2c(c1)C(c1ccccc1)(c1ccccc1)c1ccccc1-2] |
| D29 | 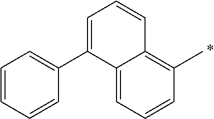 [SMILES: Cc1cccc2c(-c3ccccc3)cccc12] | 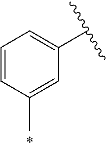 [SMILES: *c1cccc(C)c1] | 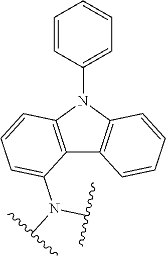 [SMILES: *N(*)c1cccc2c1c1ccccc1n2-c1ccccc1] | 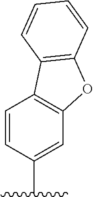 [SMILES: *c1ccc2c(c1)oc1ccccc12] |
| D30 | 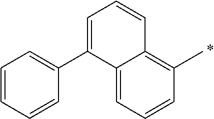 [SMILES: Cc1cccc2c(-c3ccccc3)cccc12] | 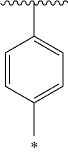 [SMILES: *c1ccc(C)cc1] | 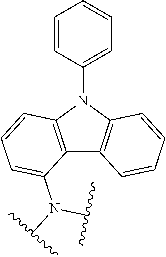 [SMILES: *N(*)c1cccc2c1c1ccccc1n2-c1ccccc1] | 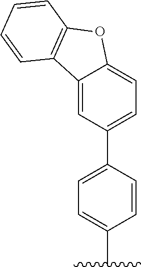 [SMILES: *c1ccc(-c2ccc3oc4ccccc4c3c2)cc1] |
| D31 | 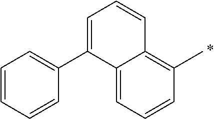 [SMILES: Cc1cccc2c(-c3ccccc3)cccc12] | 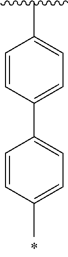 [SMILES: *c1ccc(-c2ccc(C)cc2)cc1] | 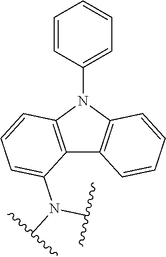 [SMILES: *N(*)c1cccc2c1c1ccccc1n2-c1ccccc1] | 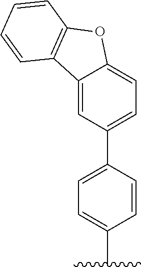 [SMILES: *c1ccc(-c2ccc3oc4ccccc4c3c2)cc1] |
| D32 | 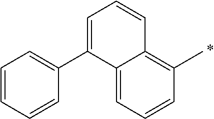 [SMILES: Cc1cccc2c(-c3ccccc3)cccc12] | 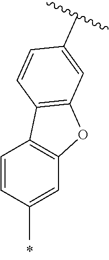 [SMILES: *c1ccc2c(c1)oc1cc(C)ccc12] | 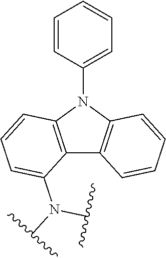 [SMILES: *N(*)c1cccc2c1c1ccccc1n2-c1ccccc1] | 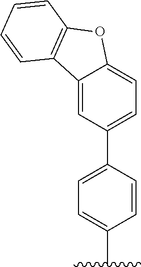 [SMILES: *c1ccc(-c2ccc3oc4ccccc4c3c2)cc1] |
| D33 | 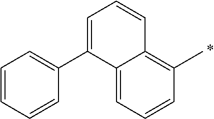 [SMILES: Cc1cccc2c(-c3ccccc3)cccc12] | 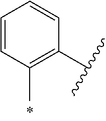 [SMILES: *c1ccccc1C] | 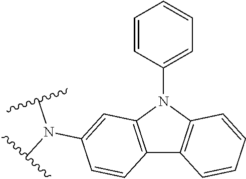 [SMILES: *N(*)c1ccc2c3ccccc3n(-c3ccccc3)c2c1] | 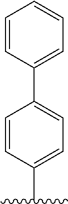 [SMILES: *c1ccc(-c2ccccc2)cc1] |
| D34 | 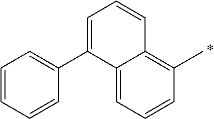 [SMILES: Cc1cccc2c(-c3ccccc3)cccc12] | 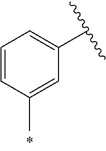 [SMILES: *c1cccc(C)c1] | 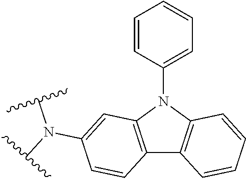 [SMILES: *N(*)c1ccc2c3ccccc3n(-c3ccccc3)c2c1] | 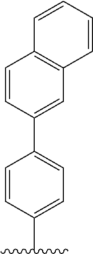 [SMILES: *c1ccc(-c2ccc3ccccc3c2)cc1] |
| D35 | 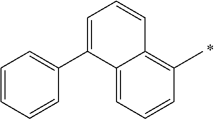 [SMILES: Cc1cccc2c(-c3ccccc3)cccc12] | 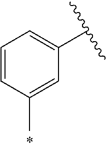 [SMILES: *c1cccc(C)c1] | 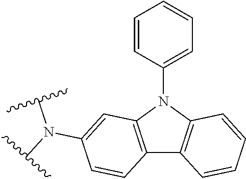 [SMILES: *N(*)c1ccc2c3ccccc3n(-c3ccccc3)c2c1] | 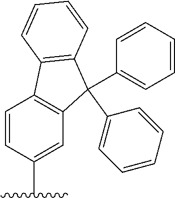 [SMILES: *c1ccc2c(c1)C(c1ccccc1)(c1ccccc1)c1ccccc1-2] |
| D36 | 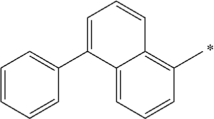 [SMILES: Cc1cccc2c(-c3ccccc3)cccc12] | 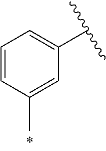 [SMILES: *c1cccc(C)c1] | 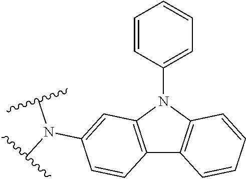 [SMILES: *N(*)c1ccc2c3ccccc3n(-c3ccccc3)c2c1] | 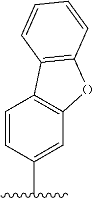 [SMILES: *c1ccc2c(c1)oc1ccccc12] |
| D37 | 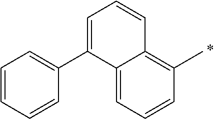 [SMILES: Cc1cccc2c(-c3ccccc3)cccc12] | 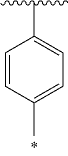 [SMILES: *c1ccc(C)cc1] | 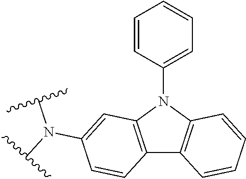 [SMILES: *N(*)c1ccc2c3ccccc3n(-c3ccccc3)c2c1] | 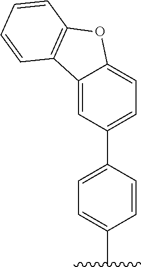 [SMILES: *c1ccc(-c2ccc3oc4ccccc4c3c2)cc1] |
| D38 | 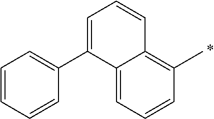 [SMILES: Cc1cccc2c(-c3ccccc3)cccc12] | 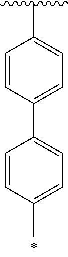 [SMILES: *c1ccc(-c2ccc(C)cc2)cc1] | 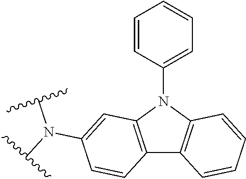 [SMILES: *N(*)c1ccc2c3ccccc3n(-c3ccccc3)c2c1] | 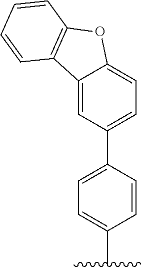 [SMILES: *c1ccc(-c2ccc3oc4ccccc4c3c2)cc1] |
| D39 | 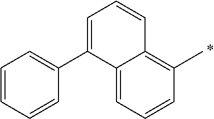 [SMILES: Cc1cccc2c(-c3ccccc3)cccc12] | 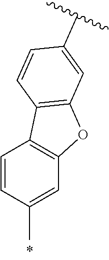 [SMILES: *c1ccc2c(c1)oc1cc(C)ccc12] | 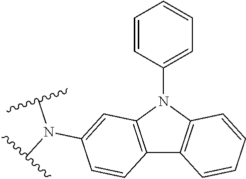 [SMILES: *N(*)c1ccc2c3ccccc3n(-c3ccccc3)c2c1] | 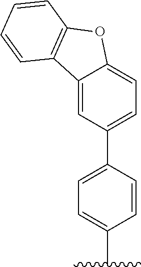 [SMILES: *c1ccc(-c2ccc3oc4ccccc4c3c2)cc1] |
| E1 | 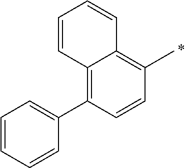 [SMILES: Cc1ccc(-c2ccccc2)c2ccccc12] | 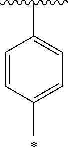 [SMILES: *c1ccc(C)cc1] | 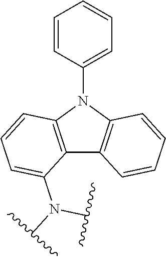 [SMILES: *N(*)c1cccc2c1c1ccccc1n2-c1ccccc1] | 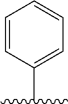 [SMILES: *c1ccccc1] |
| E2 | 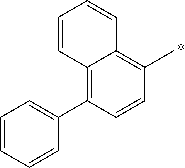 [SMILES: Cc1ccc(-c2ccccc2)c2ccccc12] | 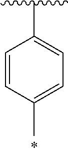 [SMILES: *c1ccc(C)cc1] | 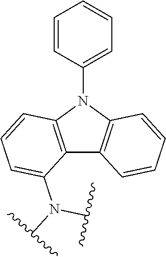 [SMILES: *N(*)c1cccc2c1c1ccccc1n2-c1ccccc1] | 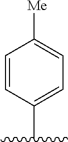 [SMILES: *c1ccc(C)cc1] |
| E3 | 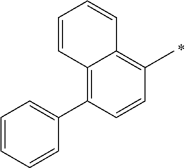 [SMILES: Cc1ccc(-c2ccccc2)c2ccccc12] | 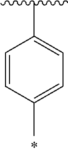 [SMILES: *c1ccc(C)cc1] | 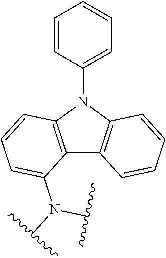 [SMILES: *N(*)c1cccc2c1c1ccccc1n2-c1ccccc1] | 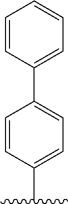 [SMILES: *c1ccc(-c2ccccc2)cc1] |
| E4 | 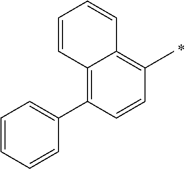 [SMILES: Cc1ccc(-c2ccccc2)c2ccccc12] | 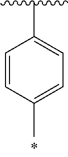 [SMILES: *c1ccc(C)cc1] | 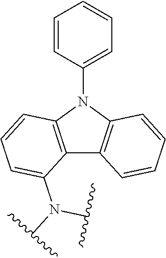 [SMILES: *N(*)c1cccc2c1c1ccccc1n2-c1ccccc1] | 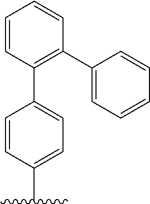 [SMILES: *c1ccc(-c2ccccc2-c2ccccc2)cc1] |
| E5 | 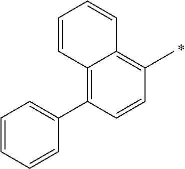 [SMILES: Cc1ccc(-c2ccccc2)c2ccccc12] | 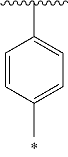 [SMILES: *c1ccc(C)cc1] | 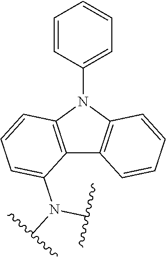 [SMILES: *N(*)c1cccc2c1c1ccccc1n2-c1ccccc1] | 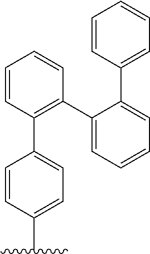 [SMILES: *c1ccc(-c2ccccc2-c2ccccc2-c2ccccc2)cc1] |
| E6 | 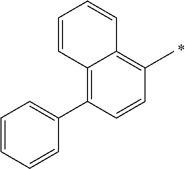 [SMILES: Cc1ccc(-c2ccccc2)c2ccccc12] | 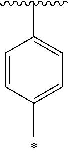 [SMILES: *c1ccc(C)cc1] | 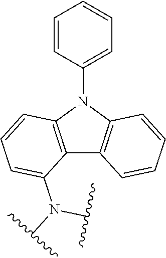 [SMILES: *N(*)c1cccc2c1c1ccccc1n2-c1ccccc1] | 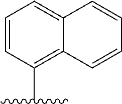 [SMILES: *c1cccc2ccccc12] |
| E7 | 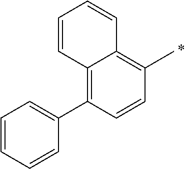 [SMILES: Cc1ccc(-c2ccccc2)c2ccccc12] | 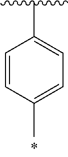 [SMILES: *c1ccc(C)cc1] | 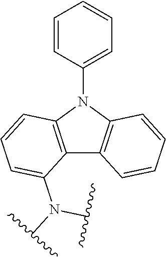 [SMILES: *N(*)c1cccc2c1c1ccccc1n2-c1ccccc1] | 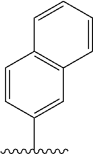 [SMILES: *c1ccc2ccccc2c1] |
| E8 | 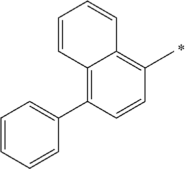 [SMILES: Cc1ccc(-c2ccccc2)c2ccccc12] | 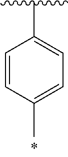 [SMILES: *c1ccc(C)cc1] | 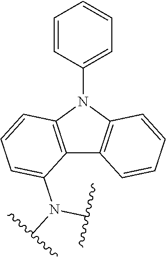 [SMILES: *N(*)c1cccc2c1c1ccccc1n2-c1ccccc1] | 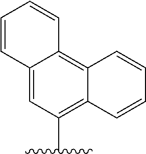 [SMILES: *c1cc2ccccc2c2ccccc12] |
| E9 | 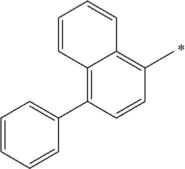 [SMILES: Cc1ccc(-c2ccccc2)c2ccccc12] | 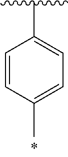 [SMILES: *c1ccc(C)cc1] | 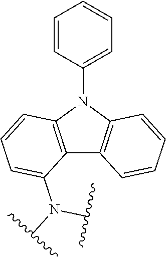 [SMILES: *N(*)c1cccc2c1c1ccccc1n2-c1ccccc1] | 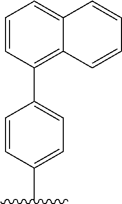 [SMILES: *c1ccc(-c2cccc3ccccc23)cc1] |
| E10 | 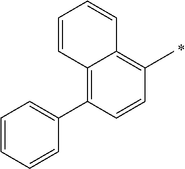 [SMILES: Cc1ccc(-c2ccccc2)c2ccccc12] | 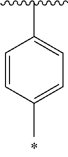 [SMILES: *c1ccc(C)cc1] | 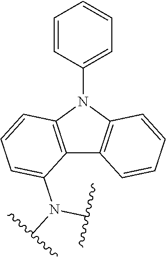 [SMILES: *N(*)c1cccc2c1c1ccccc1n2-c1ccccc1] | 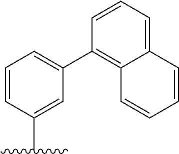 [SMILES: *c1cccc(-c2cccc3ccccc23)c1] |
| E11 | 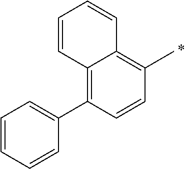 [SMILES: Cc1ccc(-c2ccccc2)c2ccccc12] | 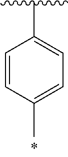 [SMILES: *c1ccc(C)cc1] | 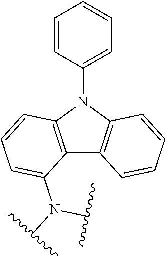 [SMILES: *N(*)c1cccc2c1c1ccccc1n2-c1ccccc1] | 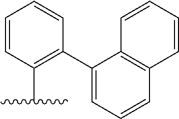 [SMILES: *c1ccccc1-c1cccc2ccccc12] |
| E12 | 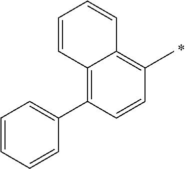 [SMILES: Cc1ccc(-c2ccccc2)c2ccccc12] | 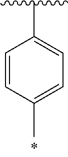 [SMILES: *c1ccc(C)cc1] | 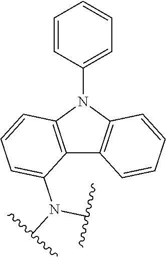 [SMILES: *N(*)c1cccc2c1c1ccccc1n2-c1ccccc1] | 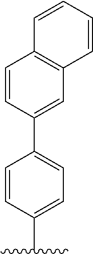 [SMILES: *c1ccc(-c2ccc3ccccc3c2)cc1] |
| E13 | 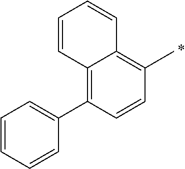 [SMILES: Cc1ccc(-c2ccccc2)c2ccccc12] | 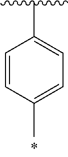 [SMILES: *c1ccc(C)cc1] | 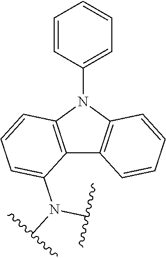 [SMILES: *N(*)c1cccc2c1c1ccccc1n2-c1ccccc1] | 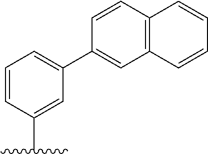 [SMILES: *c1cccc(-c2ccc3ccccc3c2)c1] |
| E14 | 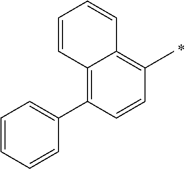 [SMILES: Cc1ccc(-c2ccccc2)c2ccccc12] | 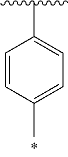 [SMILES: *c1ccc(C)cc1] | 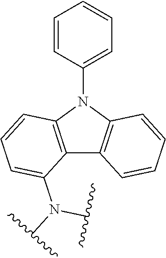 [SMILES: *N(*)c1cccc2c1c1ccccc1n2-c1ccccc1] | 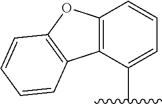 [SMILES: *c1cccc2oc3ccccc3c12] |
| E15 | 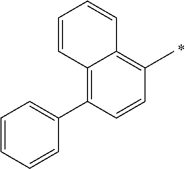 [SMILES: Cc1ccc(-c2ccccc2)c2ccccc12] | 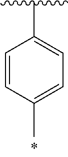 [SMILES: *c1ccc(C)cc1] | 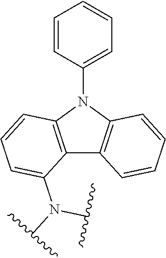 [SMILES: *N(*)c1cccc2c1c1ccccc1n2-c1ccccc1] | 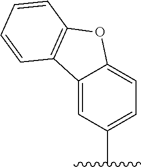 [SMILES: *c1ccc2oc3ccccc3c2c1] |
| E16 | 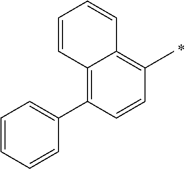 [SMILES: Cc1ccc(-c2ccccc2)c2ccccc12] | 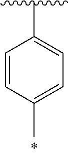 [SMILES: *c1ccc(C)cc1] | 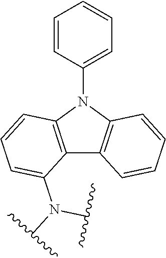 [SMILES: *N(*)c1cccc2c1c1ccccc1n2-c1ccccc1] | 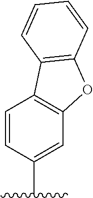 [SMILES: *c1ccc2c(c1)oc1ccccc12] |
| E17 | 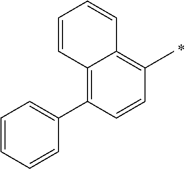 [SMILES: Cc1ccc(-c2ccccc2)c2ccccc12] | 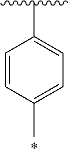 [SMILES: *c1ccc(C)cc1] | 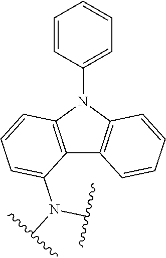 [SMILES: *N(*)c1cccc2c1c1ccccc1n2-c1ccccc1] | 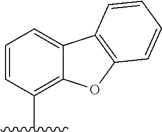 [SMILES: *c1cccc2c1oc1ccccc12] |
| E18 | 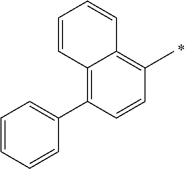 [SMILES: Cc1ccc(-c2ccccc2)c2ccccc12] | 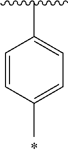 [SMILES: *c1ccc(C)cc1] | 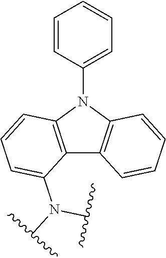 [SMILES: *N(*)c1cccc2c1c1ccccc1n2-c1ccccc1] | 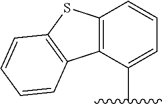 [SMILES: *c1cccc2sc3ccccc3c12] |
| E19 | 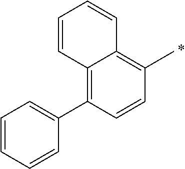 [SMILES: Cc1ccc(-c2ccccc2)c2ccccc12] | 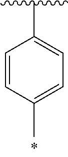 [SMILES: *c1ccc(C)cc1] | 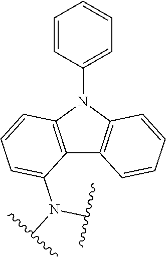 [SMILES: *N(*)c1cccc2c1c1ccccc1n2-c1ccccc1] | 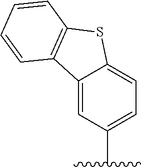 [SMILES: *c1ccc2sc3ccccc3c2c1] |
| E20 | 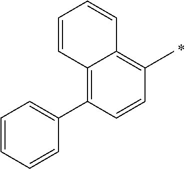 [SMILES: Cc1ccc(-c2ccccc2)c2ccccc12] | 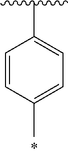 [SMILES: *c1ccc(C)cc1] | 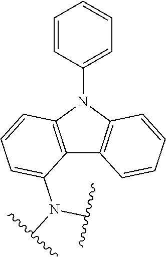 [SMILES: *N(*)c1cccc2c1c1ccccc1n2-c1ccccc1] | 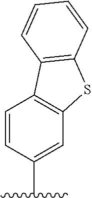 [SMILES: *c1ccc2c(c1)sc1ccccc12] |
| E21 | 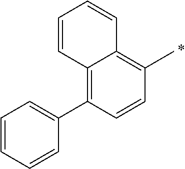 [SMILES: Cc1ccc(-c2ccccc2)c2ccccc12] | 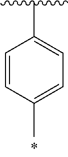 [SMILES: *c1ccc(C)cc1] | 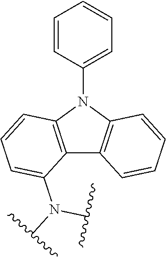 [SMILES: *N(*)c1cccc2c1c1ccccc1n2-c1ccccc1] | 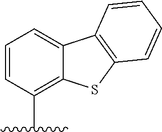 [SMILES: *c1cccc2c1sc1ccccc12] |
| E22 | 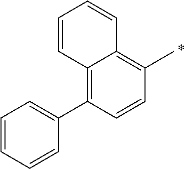 [SMILES: Cc1ccc(-c2ccccc2)c2ccccc12] | 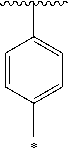 [SMILES: *c1ccc(C)cc1] | 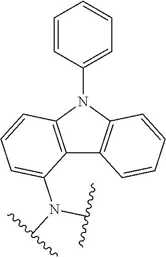 [SMILES: *N(*)c1cccc2c1c1ccccc1n2-c1ccccc1] | 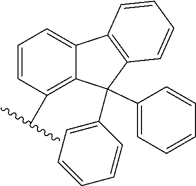 [SMILES: *c1cccc2c1C(c1ccccc1)(c1ccccc1)c1ccccc1-2] |
| E23 | 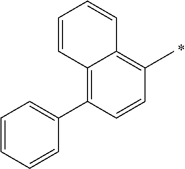 [SMILES: Cc1ccc(-c2ccccc2)c2ccccc12] | 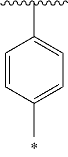 [SMILES: *c1ccc(C)cc1] | 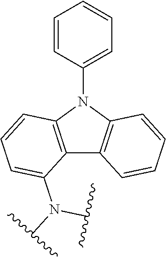 [SMILES: *N(*)c1cccc2c1c1ccccc1n2-c1ccccc1] | 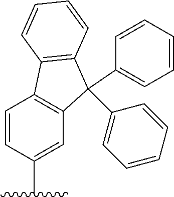 [SMILES: *c1ccc2c(c1)C(c1ccccc1)(c1ccccc1)c1ccccc1-2] |
| E24 | 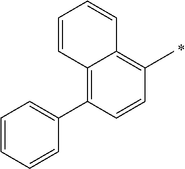 [SMILES: Cc1ccc(-c2ccccc2)c2ccccc12] | 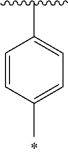 [SMILES: *c1ccc(C)cc1] | 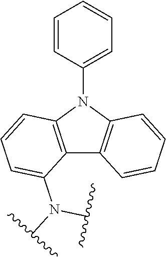 [SMILES: *N(*)c1cccc2c1c1ccccc1n2-c1ccccc1] | 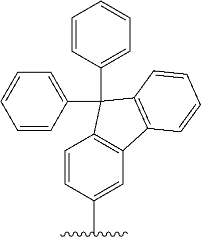 [SMILES: *c1ccc2c(c1)-c1ccccc1C2(c1ccccc1)c1ccccc1] |
| E25 | 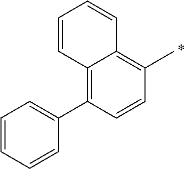 [SMILES: Cc1ccc(-c2ccccc2)c2ccccc12] | 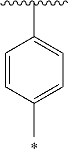 [SMILES: *c1ccc(C)cc1] | 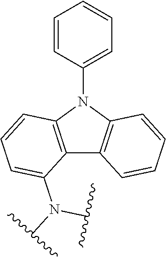 [SMILES: *N(*)c1cccc2c1c1ccccc1n2-c1ccccc1] | 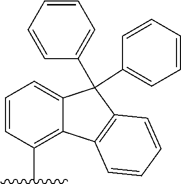 [SMILES: *c1cccc2c1-c1ccccc1C2(c1ccccc1)c1ccccc1] |
| E26 | 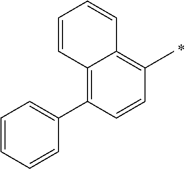 [SMILES: Cc1ccc(-c2ccccc2)c2ccccc12] | 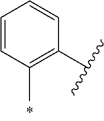 [SMILES: *c1ccccc1C] | 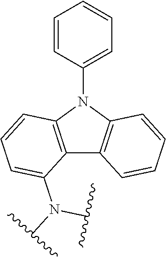 [SMILES: *N(*)c1cccc2c1c1ccccc1n2-c1ccccc1] | 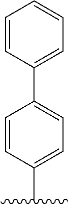 [SMILES: *c1ccc(-c2ccccc2)cc1] |
| E27 | 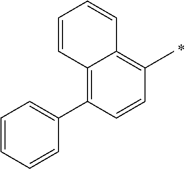 [SMILES: Cc1ccc(-c2ccccc2)c2ccccc12] | 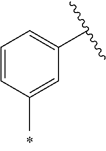 [SMILES: *c1cccc(C)c1] | 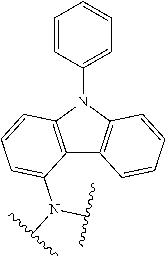 [SMILES: *N(*)c1cccc2c1c1ccccc1n2-c1ccccc1] | 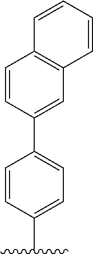 [SMILES: *c1ccc(-c2ccc3ccccc3c2)cc1] |
| E28 | 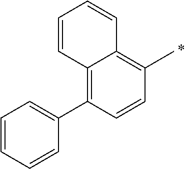 [SMILES: Cc1ccc(-c2ccccc2)c2ccccc12] | 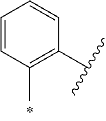 [SMILES: *c1ccccc1C] | 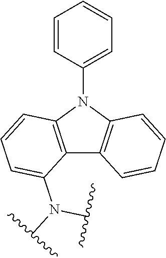 [SMILES: *N(*)c1cccc2c1c1ccccc1n2-c1ccccc1] | 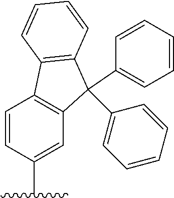 [SMILES: *c1ccc2c(c1)C(c1ccccc1)(c1ccccc1)c1ccccc1-2] |
| E29 | 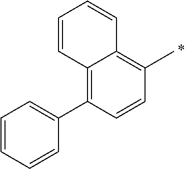 [SMILES: Cc1ccc(-c2ccccc2)c2ccccc12] | 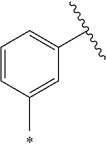 [SMILES: *c1cccc(C)c1] | 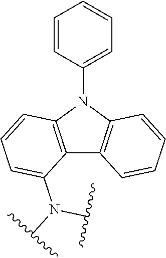 [SMILES: *N(*)c1cccc2c1c1ccccc1n2-c1ccccc1] | 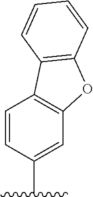 [SMILES: *c1ccc2c(c1)oc1ccccc12] |
| E30 | 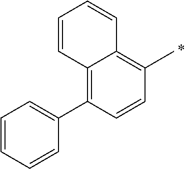 [SMILES: Cc1ccc(-c2ccccc2)c2ccccc12] | 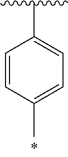 [SMILES: *c1ccc(C)cc1] | 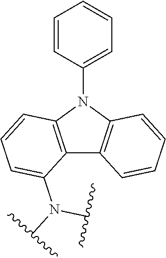 [SMILES: *N(*)c1cccc2c1c1ccccc1n2-c1ccccc1] | 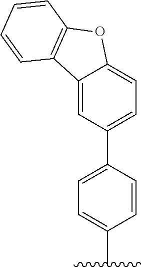 [SMILES: *c1ccc(-c2ccc3oc4ccccc4c3c2)cc1] |
| E31 | 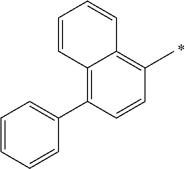 [SMILES: Cc1ccc(-c2ccccc2)c2ccccc12] | 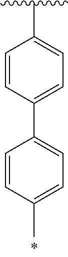 [SMILES: *c1ccc(-c2ccc(C)cc2)cc1] | 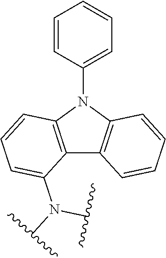 [SMILES: *N(*)c1cccc2c1c1ccccc1n2-c1ccccc1] | 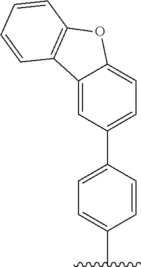 [SMILES: *c1ccc(-c2ccc3oc4ccccc4c3c2)cc1] |
| E32 | 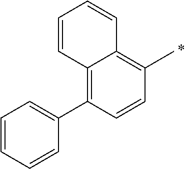 [SMILES: Cc1ccc(-c2ccccc2)c2ccccc12] | 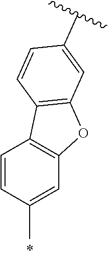 [SMILES: *c1ccc2c(c1)oc1cc(C)ccc12] | 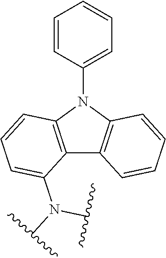 [SMILES: *N(*)c1cccc2c1c1ccccc1n2-c1ccccc1] | 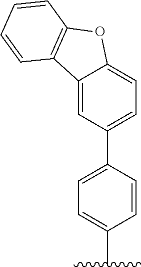 [SMILES: *c1ccc(-c2ccc3oc4ccccc4c3c2)cc1] |
| E33 | 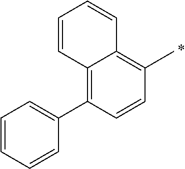 [SMILES: Cc1ccc(-c2ccccc2)c2ccccc12] | 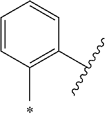 [SMILES: *c1ccccc1C] | 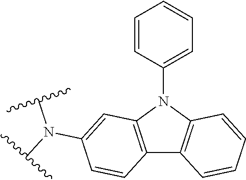 [SMILES: *N(*)c1ccc2c3ccccc3n(-c3ccccc3)c2c1] | 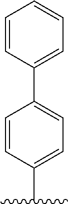 [SMILES: *c1ccc(-c2ccccc2)cc1] |
| E34 | 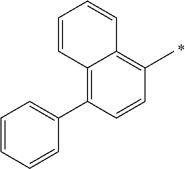 [SMILES: Cc1ccc(-c2ccccc2)c2ccccc12] | 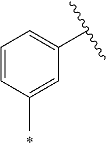 [SMILES: *c1cccc(C)c1] | 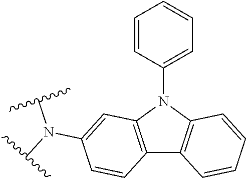 [SMILES: *N(*)c1ccc2c3ccccc3n(-c3ccccc3)c2c1] | 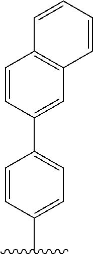 [SMILES: *c1ccc(-c2ccc3ccccc3c2)cc1] |
| E35 | 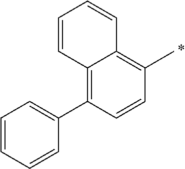 [SMILES: Cc1ccc(-c2ccccc2)c2ccccc12] | 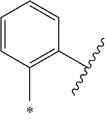 [SMILES: *c1ccccc1C] | 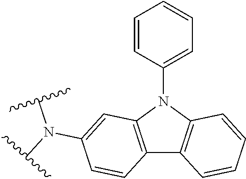 [SMILES: *N(*)c1ccc2c3ccccc3n(-c3ccccc3)c2c1] | 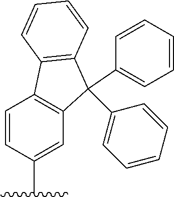 [SMILES: *c1ccc2c(c1)C(c1ccccc1)(c1ccccc1)c1ccccc1-2] |
| E36 | 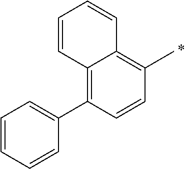 [SMILES: Cc1ccc(-c2ccccc2)c2ccccc12] | 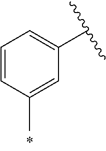 [SMILES: *c1cccc(C)c1] | 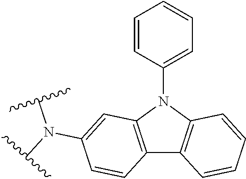 [SMILES: *N(*)c1ccc2c3ccccc3n(-c3ccccc3)c2c1] | 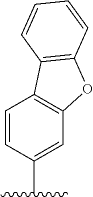 [SMILES: *c1ccc2c(c1)oc1ccccc12] |
| E37 | 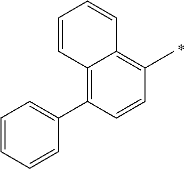 [SMILES: Cc1ccc(-c2ccccc2)c2ccccc12] | 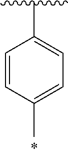 [SMILES: *c1ccc(C)cc1] | 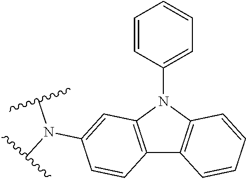 [SMILES: *N(*)c1ccc2c3ccccc3n(-c3ccccc3)c2c1] | 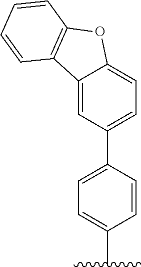 [SMILES: *c1ccc(-c2ccc3oc4ccccc4c3c2)cc1] |
| E38 | 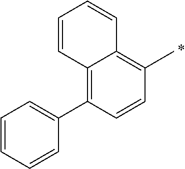 [SMILES: Cc1ccc(-c2ccccc2)c2ccccc12] | 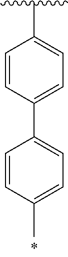 [SMILES: *c1ccc(-c2ccc(C)cc2)cc1] | 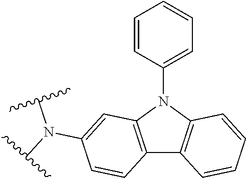 [SMILES: *N(*)c1ccc2c3ccccc3n(-c3ccccc3)c2c1] | 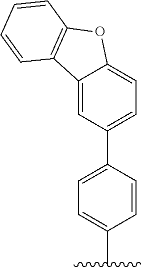 [SMILES: *c1ccc(-c2ccc3oc4ccccc4c3c2)cc1] |
| E39 | 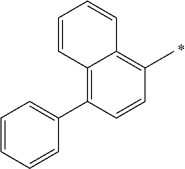 [SMILES: Cc1ccc(-c2ccccc2)c2ccccc12] | 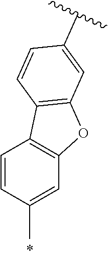 [SMILES: *c1ccc2c(c1)oc1cc(C)ccc12] | 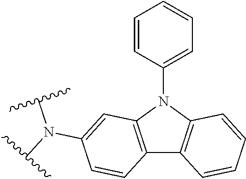 [SMILES: *N(*)c1ccc2c3ccccc3n(-c3ccccc3)c2c1] | 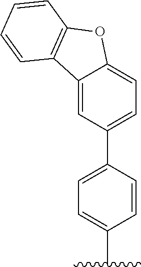 [SMILES: *c1ccc(-c2ccc3oc4ccccc4c3c2)cc1] |
| F1 | 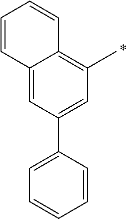 [SMILES: Cc1cc(-c2ccccc2)cc2ccccc12] | 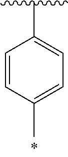 [SMILES: *c1ccc(C)cc1] | 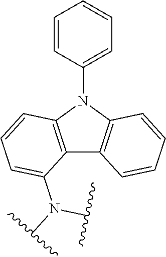 [SMILES: *N(*)c1cccc2c1c1ccccc1n2-c1ccccc1] | 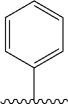 [SMILES: *c1ccccc1] |
| F2 | 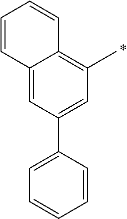 [SMILES: Cc1cc(-c2ccccc2)cc2ccccc12] | 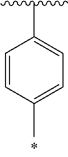 [SMILES: *c1ccc(C)cc1] | 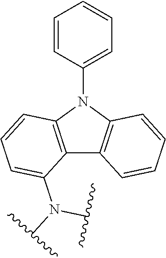 [SMILES: *N(*)c1cccc2c1c1ccccc1n2-c1ccccc1] | 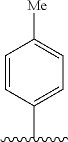 [SMILES: *c1ccc(C)cc1] |
| F3 | 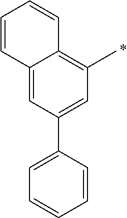 [SMILES: Cc1cc(-c2ccccc2)cc2ccccc12] | 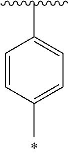 [SMILES: *c1ccc(C)cc1] | 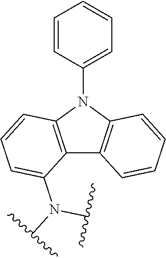 [SMILES: *N(*)c1cccc2c1c1ccccc1n2-c1ccccc1] | 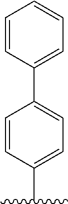 [SMILES: *c1ccc(-c2ccccc2)cc1] |
| F4 | 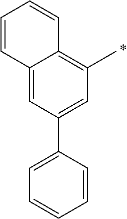 [SMILES: Cc1cc(-c2ccccc2)cc2ccccc12] | 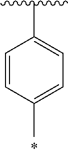 [SMILES: *c1ccc(C)cc1] | 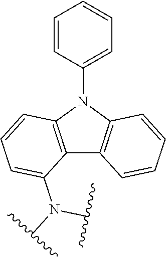 [SMILES: *N(*)c1cccc2c1c1ccccc1n2-c1ccccc1] | 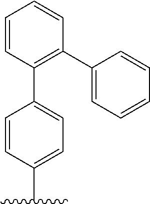 [SMILES: *c1ccc(-c2ccccc2-c2ccccc2)cc1] |
| F5 | 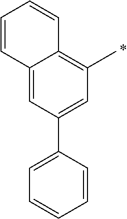 [SMILES: Cc1cc(-c2ccccc2)cc2ccccc12] | 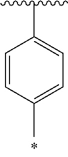 [SMILES: *c1ccc(C)cc1] | 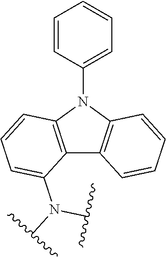 [SMILES: *N(*)c1cccc2c1c1ccccc1n2-c1ccccc1] | 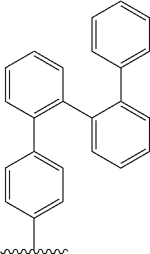 [SMILES: *c1ccc(-c2ccccc2-c2ccccc2-c2ccccc2)cc1] |
| F6 | 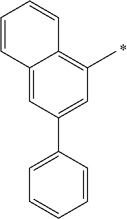 [SMILES: Cc1cc(-c2ccccc2)cc2ccccc12] | 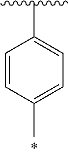 [SMILES: *c1ccc(C)cc1] | 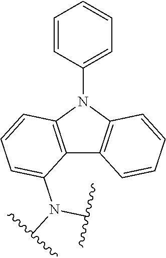 [SMILES: *N(*)c1cccc2c1c1ccccc1n2-c1ccccc1] | 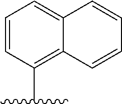 [SMILES: *c1cccc2ccccc12] |
| F7 | 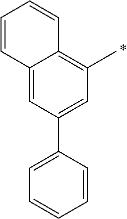 [SMILES: Cc1cc(-c2ccccc2)cc2ccccc12] | 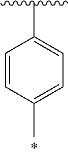 [SMILES: *c1ccc(C)cc1] | 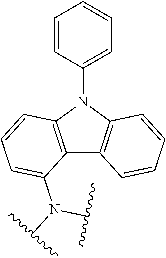 [SMILES: *N(*)c1cccc2c1c1ccccc1n2-c1ccccc1] | 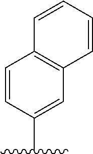 [SMILES: *c1ccc2ccccc2c1] |
| F8 | 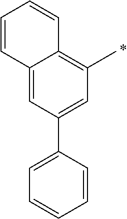 [SMILES: Cc1cc(-c2ccccc2)cc2ccccc12] | 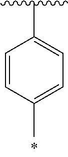 [SMILES: *c1ccc(C)cc1] | 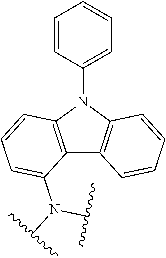 [SMILES: *N(*)c1cccc2c1c1ccccc1n2-c1ccccc1] | 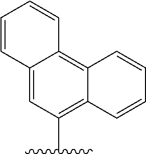 [SMILES: *c1cc2ccccc2c2ccccc12] |
| F9 | 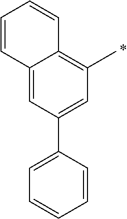 [SMILES: Cc1cc(-c2ccccc2)cc2ccccc12] | 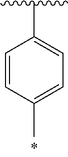 [SMILES: *c1ccc(C)cc1] | 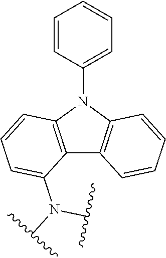 [SMILES: *N(*)c1cccc2c1c1ccccc1n2-c1ccccc1] | 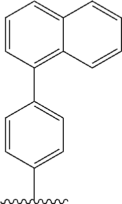 [SMILES: *c1ccc(-c2cccc3ccccc23)cc1] |
| F10 | 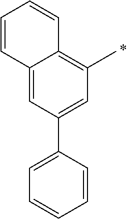 [SMILES: Cc1cc(-c2ccccc2)cc2ccccc12] | 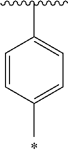 [SMILES: *c1ccc(C)cc1] | 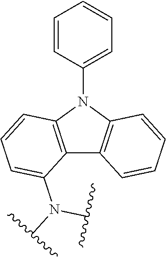 [SMILES: *N(*)c1cccc2c1c1ccccc1n2-c1ccccc1] | 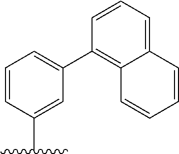 [SMILES: *c1cccc(-c2cccc3ccccc23)c1] |
| F11 | 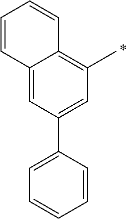 [SMILES: Cc1cc(-c2ccccc2)cc2ccccc12] | 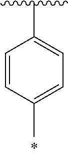 [SMILES: *c1ccc(C)cc1] | 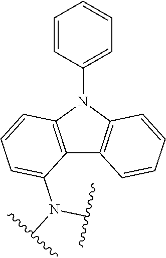 [SMILES: *N(*)c1cccc2c1c1ccccc1n2-c1ccccc1] | 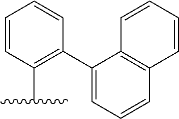 [SMILES: *c1ccccc1-c1cccc2ccccc12] |
| F12 | 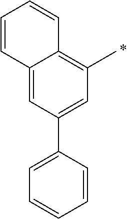 [SMILES: Cc1cc(-c2ccccc2)cc2ccccc12] | 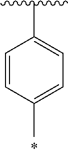 [SMILES: *c1ccc(C)cc1] | 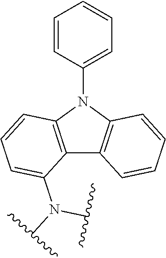 [SMILES: *N(*)c1cccc2c1c1ccccc1n2-c1ccccc1] | 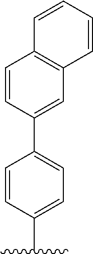 [SMILES: *c1ccc(-c2ccc3ccccc3c2)cc1] |
| F13 | 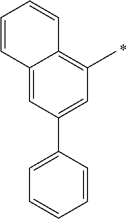 [SMILES: Cc1cc(-c2ccccc2)cc2ccccc12] | 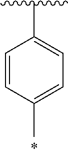 [SMILES: *c1ccc(C)cc1] | 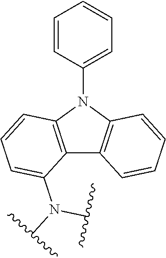 [SMILES: *N(*)c1cccc2c1c1ccccc1n2-c1ccccc1] | 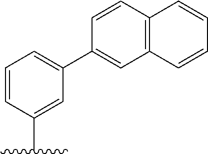 [SMILES: *c1cccc(-c2ccc3ccccc3c2)c1] |
| F14 | 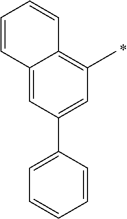 [SMILES: Cc1cc(-c2ccccc2)cc2ccccc12] | 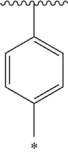 [SMILES: *c1ccc(C)cc1] | 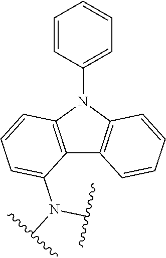 [SMILES: *N(*)c1cccc2c1c1ccccc1n2-c1ccccc1] | 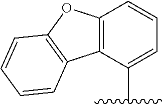 [SMILES: *c1cccc2oc3ccccc3c12] |
| F15 | 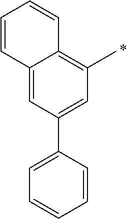 [SMILES: Cc1cc(-c2ccccc2)cc2ccccc12] | 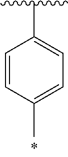 [SMILES: *c1ccc(C)cc1] | 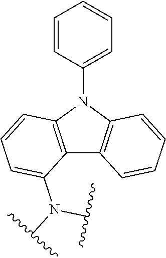 [SMILES: *N(*)c1cccc2c1c1ccccc1n2-c1ccccc1] | 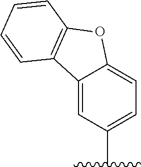 [SMILES: *c1ccc2oc3ccccc3c2c1] |
| F16 | 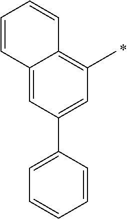 [SMILES: Cc1cc(-c2ccccc2)cc2ccccc12] | 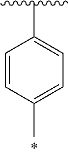 [SMILES: *c1ccc(C)cc1] | 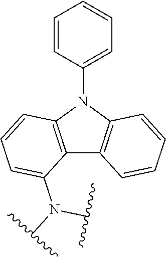 [SMILES: *N(*)c1cccc2c1c1ccccc1n2-c1ccccc1] | 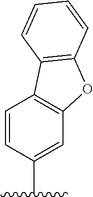 [SMILES: *c1ccc2c(c1)oc1ccccc12] |
| F17 | 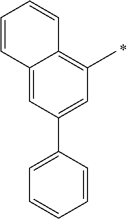 [SMILES: Cc1cc(-c2ccccc2)cc2ccccc12] | 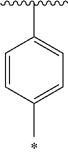 [SMILES: *c1ccc(C)cc1] | 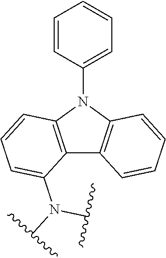 [SMILES: *N(*)c1cccc2c1c1ccccc1n2-c1ccccc1] | 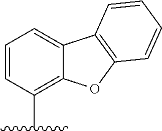 [SMILES: *c1cccc2c1oc1ccccc12] |
| F18 | 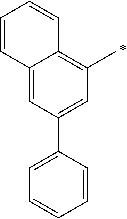 [SMILES: Cc1cc(-c2ccccc2)cc2ccccc12] | 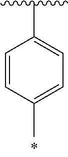 [SMILES: *c1ccc(C)cc1] | 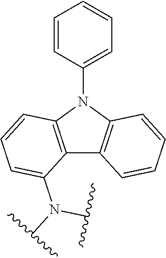 [SMILES: *N(*)c1cccc2c1c1ccccc1n2-c1ccccc1] | 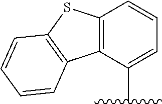 [SMILES: *c1cccc2sc3ccccc3c12] |
| F19 | 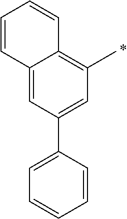 [SMILES: Cc1cc(-c2ccccc2)cc2ccccc12] | 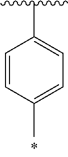 [SMILES: *c1ccc(C)cc1] | 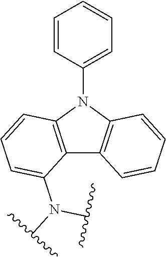 [SMILES: *N(*)c1cccc2c1c1ccccc1n2-c1ccccc1] | 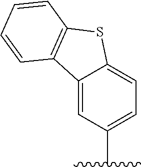 [SMILES: *c1ccc2sc3ccccc3c2c1] |
| F20 | 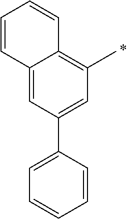 [SMILES: Cc1cc(-c2ccccc2)cc2ccccc12] | 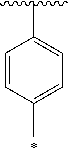 [SMILES: *c1ccc(C)cc1] | 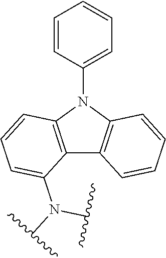 [SMILES: *N(*)c1cccc2c1c1ccccc1n2-c1ccccc1] | 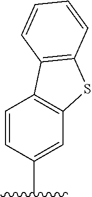 [SMILES: *c1ccc2c(c1)sc1ccccc12] |
| F21 | 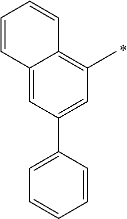 [SMILES: Cc1cc(-c2ccccc2)cc2ccccc12] | 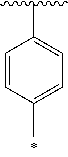 [SMILES: *c1ccc(C)cc1] | 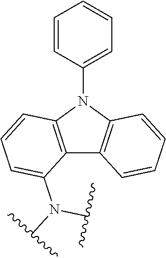 [SMILES: *N(*)c1cccc2c1c1ccccc1n2-c1ccccc1] | 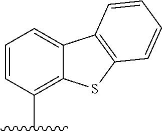 [SMILES: *c1cccc2c1sc1ccccc12] |
| F22 | 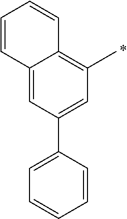 [SMILES: Cc1cc(-c2ccccc2)cc2ccccc12] | 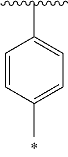 [SMILES: *c1ccc(C)cc1] | 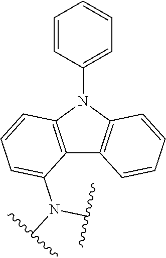 [SMILES: *N(*)c1cccc2c1c1ccccc1n2-c1ccccc1] | 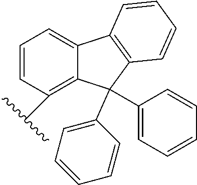 [SMILES: *c1cccc2c1C(c1ccccc1)(c1ccccc1)c1ccccc1-2] |
| F23 | 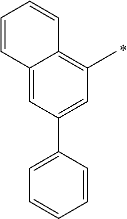 [SMILES: Cc1cc(-c2ccccc2)cc2ccccc12] | 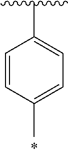 [SMILES: *c1ccc(C)cc1] | 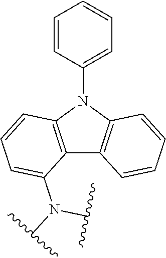 [SMILES: *N(*)c1cccc2c1c1ccccc1n2-c1ccccc1] | 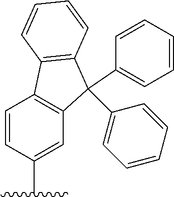 [SMILES: *c1ccc2c(c1)C(c1ccccc1)(c1ccccc1)c1ccccc1-2] |
| F24 | 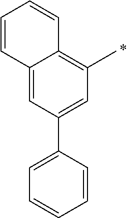 [SMILES: Cc1cc(-c2ccccc2)cc2ccccc12] | 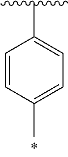 [SMILES: *c1ccc(C)cc1] | 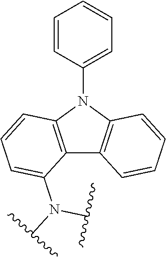 [SMILES: *N(*)c1cccc2c1c1ccccc1n2-c1ccccc1] | 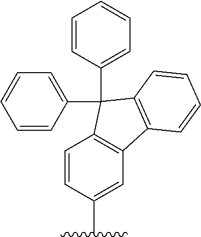 [SMILES: *c1ccc2c(c1)-c1ccccc1C2(c1ccccc1)c1ccccc1] |
| F25 | 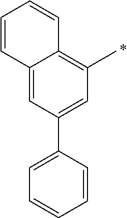 [SMILES: Cc1cc(-c2ccccc2)cc2ccccc12] | 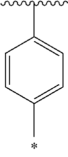 [SMILES: *c1ccc(C)cc1] | 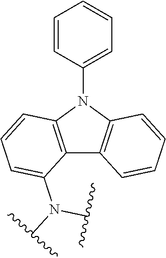 [SMILES: *N(*)c1cccc2c1c1ccccc1n2-c1ccccc1] | 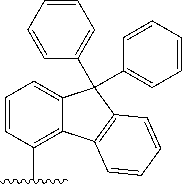 [SMILES: *c1cccc2c1-c1ccccc1C2(c1ccccc1)c1ccccc1] |
| F26 | 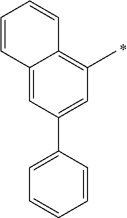 [SMILES: Cc1cc(-c2ccccc2)cc2ccccc12] | 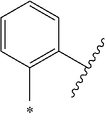 [SMILES: *c1ccccc1C] | 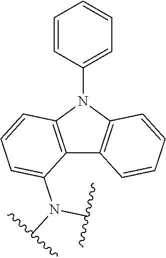 [SMILES: *N(*)c1cccc2c1c1ccccc1n2-c1ccccc1] | 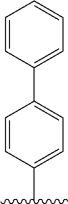 [SMILES: *c1ccc(-c2ccccc2)cc1] |
| F27 | 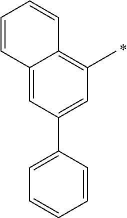 [SMILES: Cc1cc(-c2ccccc2)cc2ccccc12] | 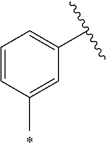 [SMILES: *c1cccc(C)c1] | 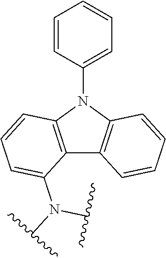 [SMILES: *N(*)c1cccc2c1c1ccccc1n2-c1ccccc1] | 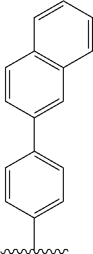 [SMILES: *c1ccc(-c2ccc3ccccc3c2)cc1] |
| F28 | 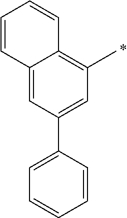 [SMILES: Cc1cc(-c2ccccc2)cc2ccccc12] | 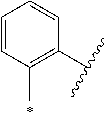 [SMILES: *c1ccccc1C] | 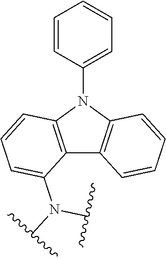 [SMILES: *N(*)c1cccc2c1c1ccccc1n2-c1ccccc1] | 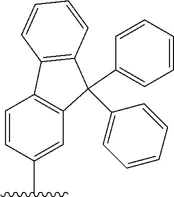 [SMILES: *c1ccc2c(c1)C(c1ccccc1)(c1ccccc1)c1ccccc1-2] |
| F29 | 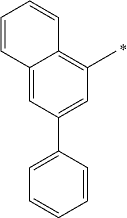 [SMILES: Cc1cc(-c2ccccc2)cc2ccccc12] | 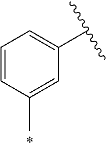 [SMILES: *c1cccc(C)c1] | 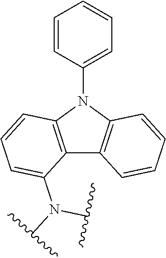 [SMILES: *N(*)c1cccc2c1c1ccccc1n2-c1ccccc1] | 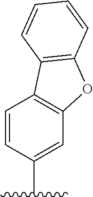 [SMILES: *c1ccc2c(c1)oc1ccccc12] |
| F30 | 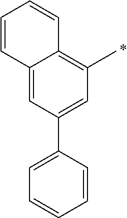 [SMILES: Cc1cc(-c2ccccc2)cc2ccccc12] | 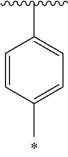 [SMILES: *c1ccc(C)cc1] | 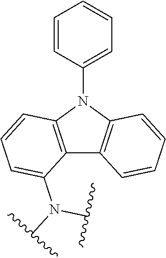 [SMILES: *N(*)c1cccc2c1c1ccccc1n2-c1ccccc1] | 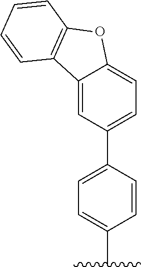 [SMILES: *c1ccc(-c2ccc3oc4ccccc4c3c2)cc1] |
| F31 | 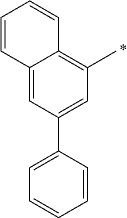 [SMILES: Cc1cc(-c2ccccc2)cc2ccccc12] | 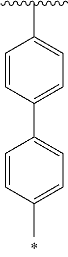 [SMILES: *c1ccc(-c2ccc(C)cc2)cc1] | 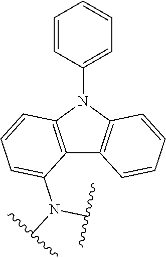 [SMILES: *N(*)c1cccc2c1c1ccccc1n2-c1ccccc1] | 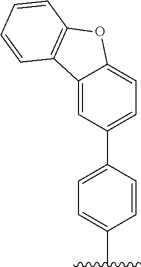 [SMILES: *c1ccc(-c2ccc3oc4ccccc4c3c2)cc1] |
| F32 | 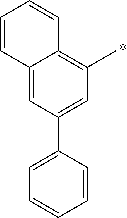 [SMILES: Cc1cc(-c2ccccc2)cc2ccccc12] | 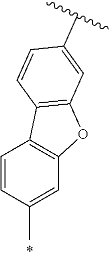 [SMILES: *c1ccc2c(c1)oc1cc(C)ccc12] | 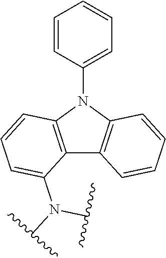 [SMILES: *N(*)c1cccc2c1c1ccccc1n2-c1ccccc1] | 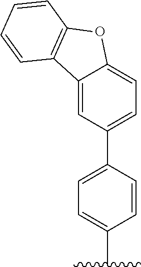 [SMILES: *c1ccc(-c2ccc3oc4ccccc4c3c2)cc1] |
| F33 | 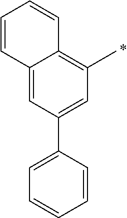 [SMILES: Cc1cc(-c2ccccc2)cc2ccccc12] | 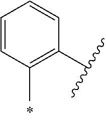 [SMILES: *c1ccccc1C] | 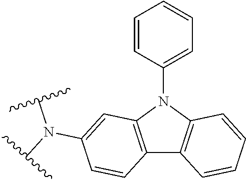 [SMILES: *N(*)c1ccc2c3ccccc3n(-c3ccccc3)c2c1] | 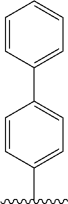 [SMILES: *c1ccc(-c2ccccc2)cc1] |
| F34 | 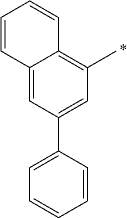 [SMILES: Cc1cc(-c2ccccc2)cc2ccccc12] | 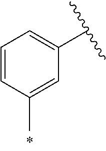 [SMILES: *c1cccc(C)c1] | 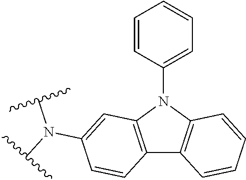 [SMILES: *N(*)c1ccc2c3ccccc3n(-c3ccccc3)c2c1] | 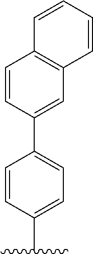 [SMILES: *c1ccc(-c2ccc3ccccc3c2)cc1] |
| F35 | 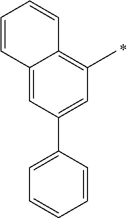 [SMILES: Cc1cc(-c2ccccc2)cc2ccccc12] | 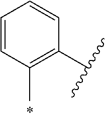 [SMILES: *c1ccccc1C] | 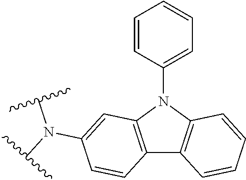 [SMILES: *N(*)c1ccc2c3ccccc3n(-c3ccccc3)c2c1] | 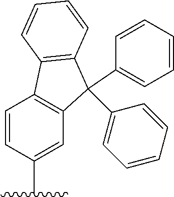 [SMILES: *c1ccc2c(c1)C(c1ccccc1)(c1ccccc1)c1ccccc1-2] |
| F36 | 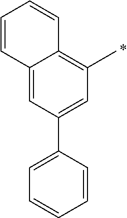 [SMILES: Cc1cc(-c2ccccc2)cc2ccccc12] | 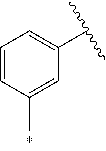 [SMILES: *c1cccc(C)c1] | 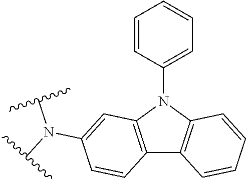 [SMILES: *N(*)c1ccc2c3ccccc3n(-c3ccccc3)c2c1] | 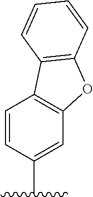 [SMILES: *c1ccc2c(c1)oc1ccccc12] |
| F37 | 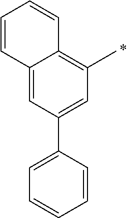 [SMILES: Cc1cc(-c2ccccc2)cc2ccccc12] | 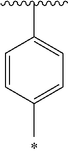 [SMILES: *c1ccc(C)cc1] | 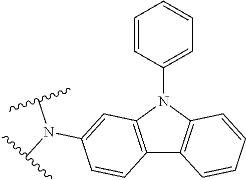 [SMILES: *N(*)c1ccc2c3ccccc3n(-c3ccccc3)c2c1] | 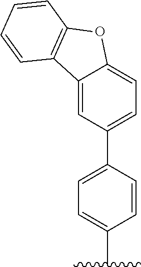 [SMILES: *c1ccc(-c2ccc3oc4ccccc4c3c2)cc1] |
| F38 | 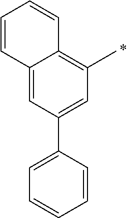 [SMILES: Cc1cc(-c2ccccc2)cc2ccccc12] | 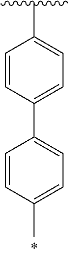 [SMILES: *c1ccc(-c2ccc(C)cc2)cc1] | 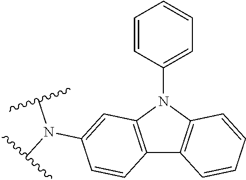 [SMILES: *N(*)c1ccc2c3ccccc3n(-c3ccccc3)c2c1] | 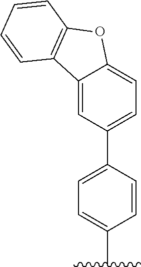 [SMILES: *c1ccc(-c2ccc3oc4ccccc4c3c2)cc1] |
| F39 | 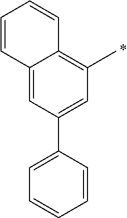 [SMILES: Cc1cc(-c2ccccc2)cc2ccccc12] | 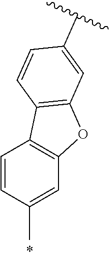 [SMILES: *c1ccc2c(c1)oc1cc(C)ccc12] | 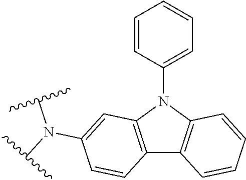 [SMILES: *N(*)c1ccc2c3ccccc3n(-c3ccccc3)c2c1] | 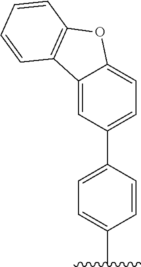 [SMILES: *c1ccc(-c2ccc3oc4ccccc4c3c2)cc1] |
| G1 | 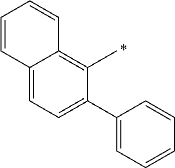 [SMILES: Cc1c(-c2ccccc2)ccc2ccccc12] | 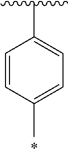 [SMILES: *c1ccc(C)cc1] | 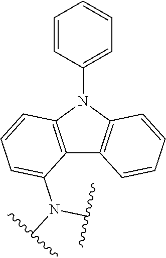 [SMILES: *N(*)c1cccc2c1c1ccccc1n2-c1ccccc1] | 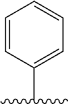 [SMILES: *c1ccccc1] |
| G2 | 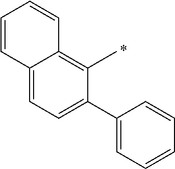 [SMILES: Cc1c(-c2ccccc2)ccc2ccccc12] | 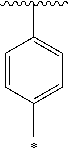 [SMILES: *c1ccc(C)cc1] | 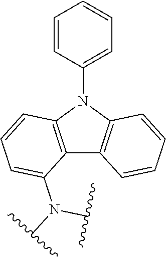 [SMILES: *N(*)c1cccc2c1c1ccccc1n2-c1ccccc1] | 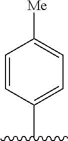 [SMILES: *c1ccc(C)cc1] |
| G3 | 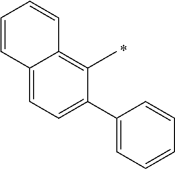 [SMILES: Cc1c(-c2ccccc2)ccc2ccccc12] | 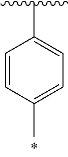 [SMILES: *c1ccc(C)cc1] | 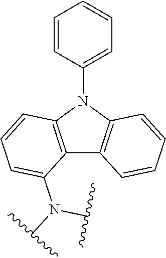 [SMILES: *N(*)c1cccc2c1c1ccccc1n2-c1ccccc1] | 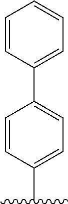 [SMILES: *c1ccc(-c2ccccc2)cc1] |
| G4 | 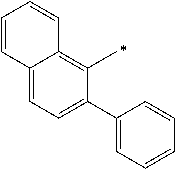 [SMILES: Cc1c(-c2ccccc2)ccc2ccccc12] | 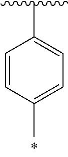 [SMILES: *c1ccc(C)cc1] | 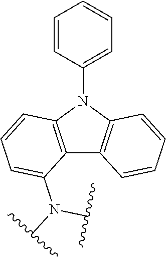 [SMILES: *N(*)c1cccc2c1c1ccccc1n2-c1ccccc1] | 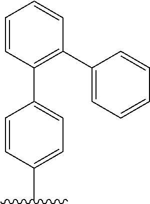 [SMILES: *c1ccc(-c2ccccc2-c2ccccc2)cc1] |
| G5 | 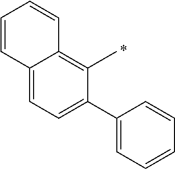 [SMILES: Cc1c(-c2ccccc2)ccc2ccccc12] | 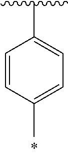 [SMILES: *c1ccc(C)cc1] | 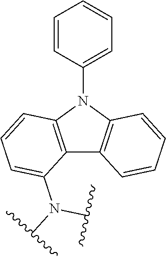 [SMILES: *N(*)c1cccc2c1c1ccccc1n2-c1ccccc1] | 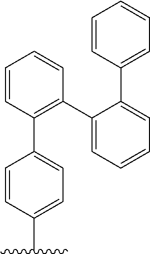 [SMILES: *c1ccc(-c2ccccc2-c2ccccc2-c2ccccc2)cc1] |
| G6 | 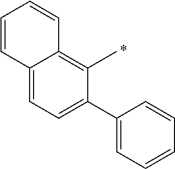 [SMILES: Cc1c(-c2ccccc2)ccc2ccccc12] | 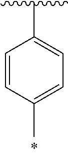 [SMILES: *c1ccc(C)cc1] | 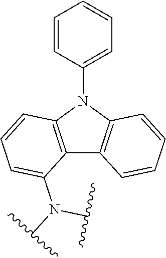 [SMILES: *N(*)c1cccc2c1c1ccccc1n2-c1ccccc1] | 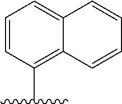 [SMILES: *c1cccc2ccccc12] |
| G7 | 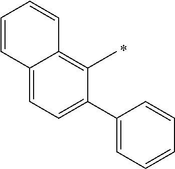 [SMILES: Cc1c(-c2ccccc2)ccc2ccccc12] | 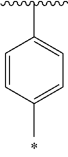 [SMILES: *c1ccc(C)cc1] | 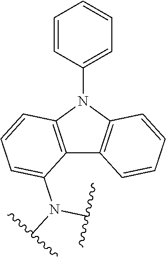 [SMILES: *N(*)c1cccc2c1c1ccccc1n2-c1ccccc1] | 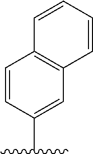 [SMILES: *c1ccc2ccccc2c1] |
| G8 | 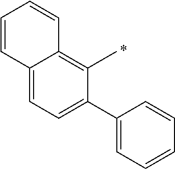 [SMILES: Cc1c(-c2ccccc2)ccc2ccccc12] | 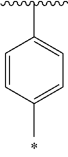 [SMILES: *c1ccc(C)cc1] | 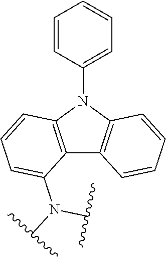 [SMILES: *N(*)c1cccc2c1c1ccccc1n2-c1ccccc1] | 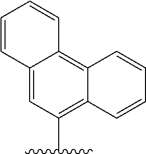 [SMILES: *c1cc2ccccc2c2ccccc12] |
| G9 | 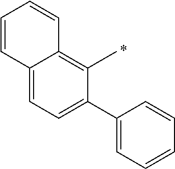 [SMILES: Cc1c(-c2ccccc2)ccc2ccccc12] | 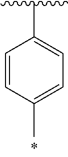 [SMILES: *c1ccc(C)cc1] | 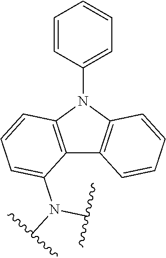 [SMILES: *N(*)c1cccc2c1c1ccccc1n2-c1ccccc1] | 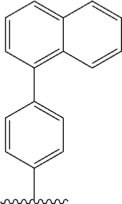 [SMILES: *c1ccc(-c2cccc3ccccc23)cc1] |
| G10 | 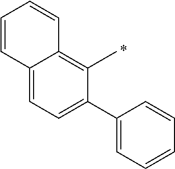 [SMILES: Cc1c(-c2ccccc2)ccc2ccccc12] | 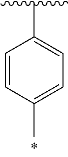 [SMILES: *c1ccc(C)cc1] | 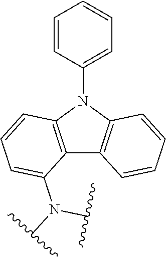 [SMILES: *N(*)c1cccc2c1c1ccccc1n2-c1ccccc1] | 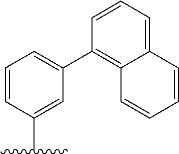 [SMILES: *c1cccc(-c2cccc3ccccc23)c1] |
| G11 | 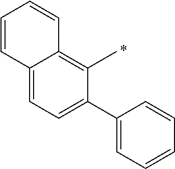 [SMILES: Cc1c(-c2ccccc2)ccc2ccccc12] | 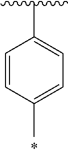 [SMILES: *c1ccc(C)cc1] | 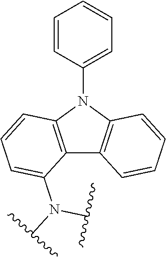 [SMILES: *N(*)c1cccc2c1c1ccccc1n2-c1ccccc1] | 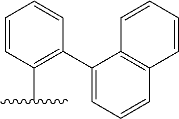 [SMILES: *c1ccccc1-c1cccc2ccccc12] |
| G12 | 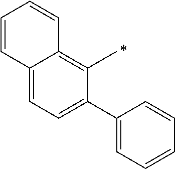 [SMILES: Cc1c(-c2ccccc2)ccc2ccccc12] | 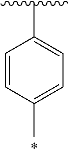 [SMILES: *c1ccc(C)cc1] | 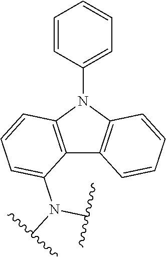 [SMILES: *N(*)c1cccc2c1c1ccccc1n2-c1ccccc1] | 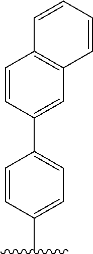 [SMILES: *c1ccc(-c2ccc3ccccc3c2)cc1] |
| G13 | 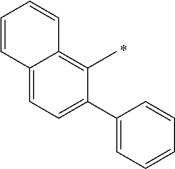 [SMILES: Cc1c(-c2ccccc2)ccc2ccccc12] | 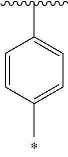 [SMILES: *c1ccc(C)cc1] | 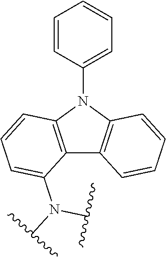 [SMILES: *N(*)c1cccc2c1c1ccccc1n2-c1ccccc1] | 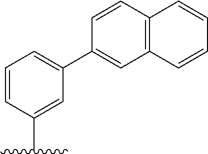 [SMILES: *c1cccc(-c2ccc3ccccc3c2)c1] |
| G14 | 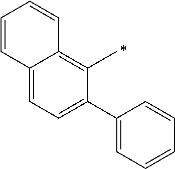 [SMILES: Cc1c(-c2ccccc2)ccc2ccccc12] | 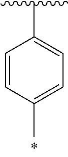 [SMILES: *c1ccc(C)cc1] | 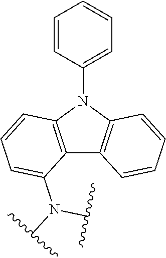 [SMILES: *N(*)c1cccc2c1c1ccccc1n2-c1ccccc1] | 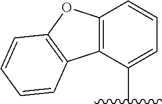 [SMILES: *c1cccc2oc3ccccc3c12] |
| G15 | 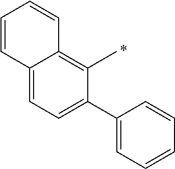 [SMILES: Cc1c(-c2ccccc2)ccc2ccccc12] | 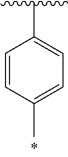 [SMILES: *c1ccc(C)cc1] | 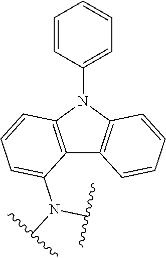 [SMILES: *N(*)c1cccc2c1c1ccccc1n2-c1ccccc1] | 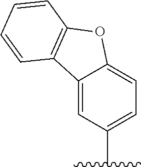 [SMILES: *c1ccc2oc3ccccc3c2c1] |
| G16 | 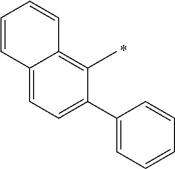 [SMILES: Cc1c(-c2ccccc2)ccc2ccccc12] | 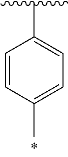 [SMILES: *c1ccc(C)cc1] | 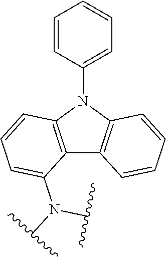 [SMILES: *N(*)c1cccc2c1c1ccccc1n2-c1ccccc1] | 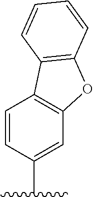 [SMILES: *c1ccc2c(c1)oc1ccccc12] |
| G17 | 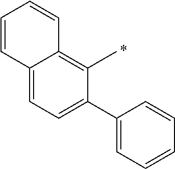 [SMILES: Cc1c(-c2ccccc2)ccc2ccccc12] | 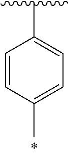 [SMILES: *c1ccc(C)cc1] | 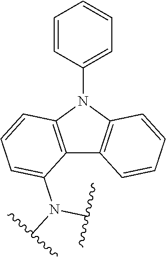 [SMILES: *N(*)c1cccc2c1c1ccccc1n2-c1ccccc1] | 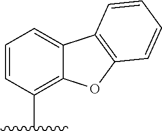 [SMILES: *c1cccc2c1oc1ccccc12] |
| G18 | 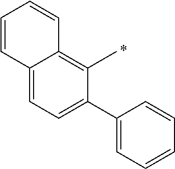 [SMILES: Cc1c(-c2ccccc2)ccc2ccccc12] | 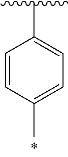 [SMILES: *c1ccc(C)cc1] | 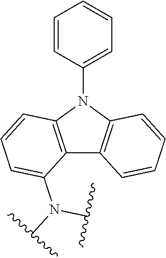 [SMILES: *N(*)c1cccc2c1c1ccccc1n2-c1ccccc1] | 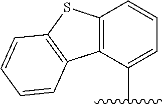 [SMILES: *c1cccc2sc3ccccc3c12] |
| G19 | 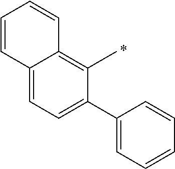 [SMILES: Cc1c(-c2ccccc2)ccc2ccccc12] | 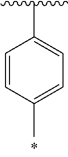 [SMILES: *c1ccc(C)cc1] | 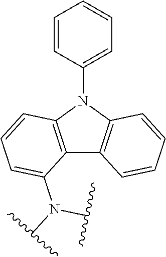 [SMILES: *N(*)c1cccc2c1c1ccccc1n2-c1ccccc1] | 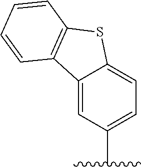 [SMILES: *c1ccc2sc3ccccc3c2c1] |
| G20 | 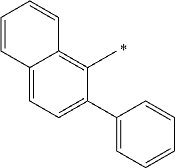 [SMILES: Cc1c(-c2ccccc2)ccc2ccccc12] | 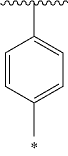 [SMILES: *c1ccc(C)cc1] | 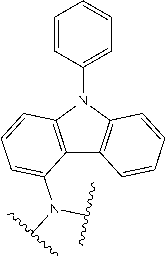 [SMILES: *N(*)c1cccc2c1c1ccccc1n2-c1ccccc1] | 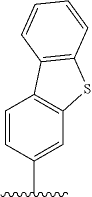 [SMILES: *c1ccc2c(c1)sc1ccccc12] |
| G21 | 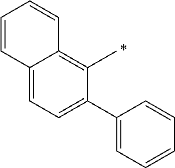 [SMILES: Cc1c(-c2ccccc2)ccc2ccccc12] | 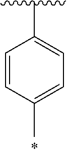 [SMILES: *c1ccc(C)cc1] | 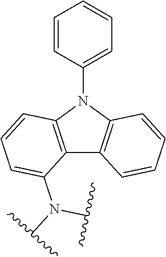 [SMILES: *N(*)c1cccc2c1c1ccccc1n2-c1ccccc1] | 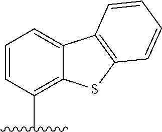 [SMILES: *c1cccc2c1sc1ccccc12] |
| G22 | 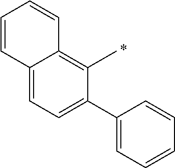 [SMILES: Cc1c(-c2ccccc2)ccc2ccccc12] | 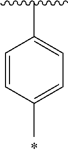 [SMILES: *c1ccc(C)cc1] | 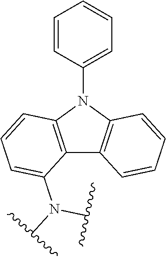 [SMILES: *N(*)c1cccc2c1c1ccccc1n2-c1ccccc1] | 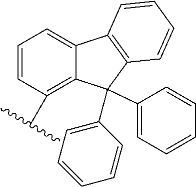 [SMILES: *c1cccc2c1C(c1ccccc1)(c1ccccc1)c1ccccc1-2] |
| G23 | 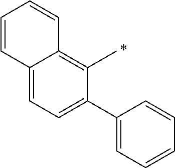 [SMILES: Cc1c(-c2ccccc2)ccc2ccccc12] | 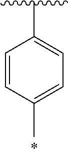 [SMILES: *c1ccc(C)cc1] | 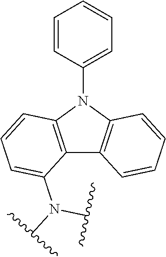 [SMILES: *N(*)c1cccc2c1c1ccccc1n2-c1ccccc1] | 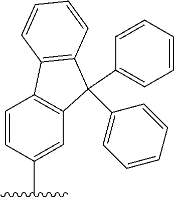 [SMILES: *c1ccc2c(c1)C(c1ccccc1)(c1ccccc1)c1ccccc1-2] |
| G24 | 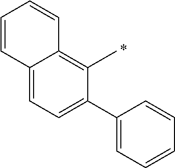 [SMILES: Cc1c(-c2ccccc2)ccc2ccccc12] | 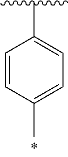 [SMILES: *c1ccc(C)cc1] | 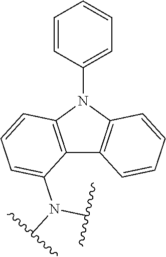 [SMILES: *N(*)c1cccc2c1c1ccccc1n2-c1ccccc1] | 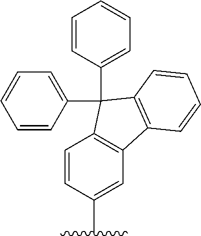 [SMILES: *c1ccc2c(c1)-c1ccccc1C2(c1ccccc1)c1ccccc1] |
| G25 | 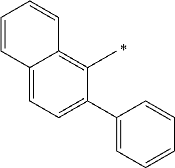 [SMILES: Cc1c(-c2ccccc2)ccc2ccccc12] | 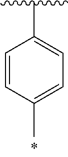 [SMILES: *c1ccc(C)cc1] | 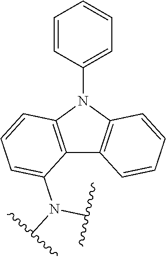 [SMILES: *N(*)c1cccc2c1c1ccccc1n2-c1ccccc1] | 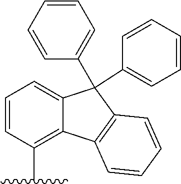 [SMILES: *c1cccc2c1-c1ccccc1C2(c1ccccc1)c1ccccc1] |
| G26 | 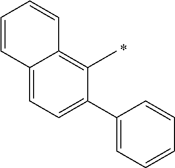 [SMILES: Cc1c(-c2ccccc2)ccc2ccccc12] | 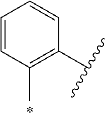 [SMILES: *c1ccccc1C] | 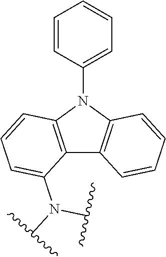 [SMILES: *N(*)c1cccc2c1c1ccccc1n2-c1ccccc1] | 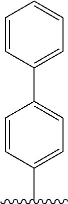 [SMILES: *c1ccc(-c2ccccc2)cc1] |
| G27 | 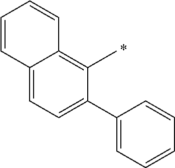 [SMILES: Cc1c(-c2ccccc2)ccc2ccccc12] | 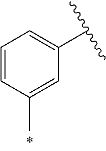 [SMILES: *c1cccc(C)c1] | 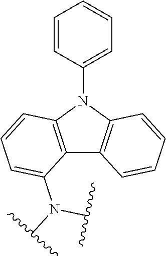 [SMILES: *N(*)c1cccc2c1c1ccccc1n2-c1ccccc1] | 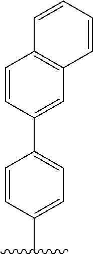 [SMILES: *c1ccc(-c2ccc3ccccc3c2)cc1] |
| G28 | 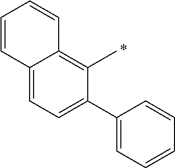 [SMILES: Cc1c(-c2ccccc2)ccc2ccccc12] | 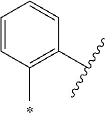 [SMILES: *c1ccccc1C] | 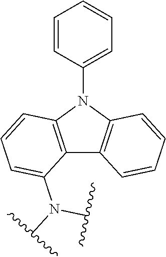 [SMILES: *N(*)c1cccc2c1c1ccccc1n2-c1ccccc1] | 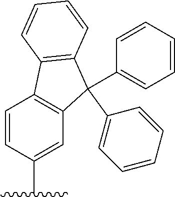 [SMILES: *c1ccc2c(c1)C(c1ccccc1)(c1ccccc1)c1ccccc1-2] |
| G29 | 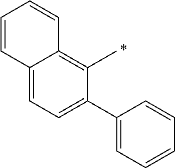 [SMILES: Cc1c(-c2ccccc2)ccc2ccccc12] | 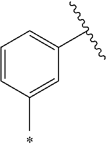 [SMILES: *c1cccc(C)c1] | 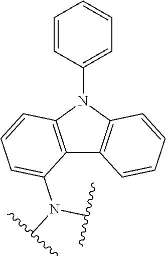 [SMILES: *N(*)c1cccc2c1c1ccccc1n2-c1ccccc1] | 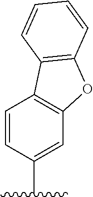 [SMILES: *c1ccc2c(c1)oc1ccccc12] |
| G30 | 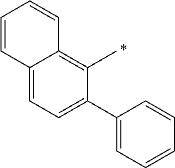 [SMILES: Cc1c(-c2ccccc2)ccc2ccccc12] | 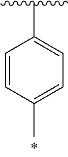 [SMILES: *c1ccc(C)cc1] | 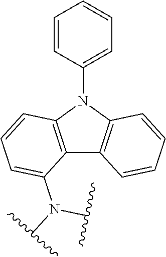 [SMILES: *N(*)c1cccc2c1c1ccccc1n2-c1ccccc1] | 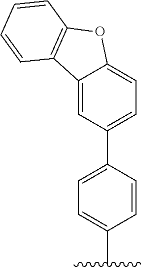 [SMILES: *c1ccc(-c2ccc3oc4ccccc4c3c2)cc1] |
| G31 | 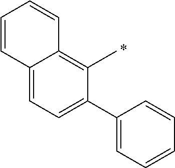 [SMILES: Cc1c(-c2ccccc2)ccc2ccccc12] | 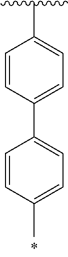 [SMILES: *c1ccc(-c2ccc(C)cc2)cc1] | 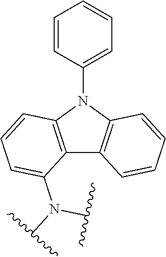 [SMILES: *N(*)c1cccc2c1c1ccccc1n2-c1ccccc1] | 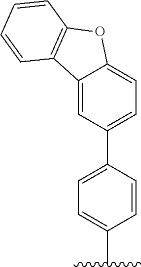 [SMILES: *c1ccc(-c2ccc3oc4ccccc4c3c2)cc1] |
| G32 | 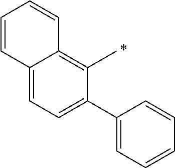 [SMILES: Cc1c(-c2ccccc2)ccc2ccccc12] | 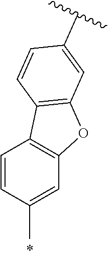 [SMILES: *c1ccc2c(c1)oc1cc(C)ccc12] | 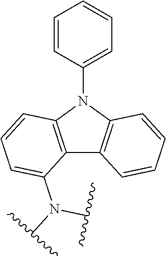 [SMILES: *N(*)c1cccc2c1c1ccccc1n2-c1ccccc1] | 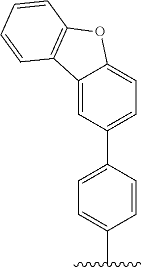 [SMILES: *c1ccc(-c2ccc3oc4ccccc4c3c2)cc1] |
| G33 | 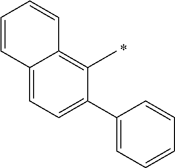 [SMILES: Cc1c(-c2ccccc2)ccc2ccccc12] | 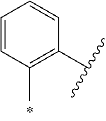 [SMILES: *c1ccccc1C] | 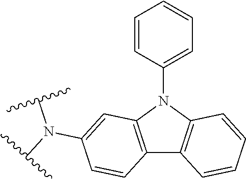 [SMILES: *N(*)c1ccc2c3ccccc3n(-c3ccccc3)c2c1] | 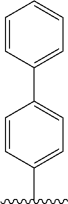 [SMILES: *c1ccc(-c2ccccc2)cc1] |
| G34 | 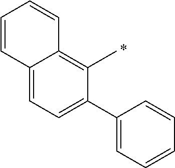 [SMILES: Cc1c(-c2ccccc2)ccc2ccccc12] | 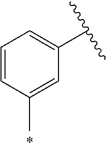 [SMILES: *c1cccc(C)c1] | 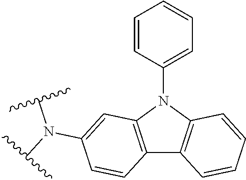 [SMILES: *N(*)c1ccc2c3ccccc3n(-c3ccccc3)c2c1] | 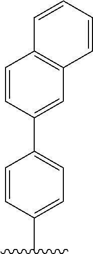 [SMILES: *c1ccc(-c2ccc3ccccc3c2)cc1] |
| G35 | 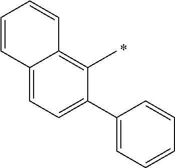 [SMILES: Cc1c(-c2ccccc2)ccc2ccccc12] | 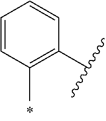 [SMILES: *c1ccccc1C] | 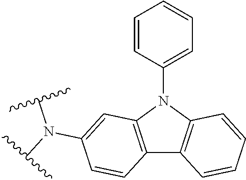 [SMILES: *N(*)c1ccc2c3ccccc3n(-c3ccccc3)c2c1] | 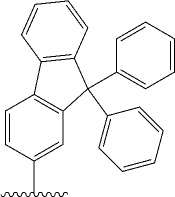 [SMILES: *c1ccc2c(c1)C(c1ccccc1)(c1ccccc1)c1ccccc1-2] |
| G36 | 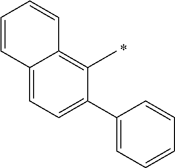 [SMILES: Cc1c(-c2ccccc2)ccc2ccccc12] | 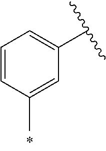 [SMILES: *c1cccc(C)c1] | 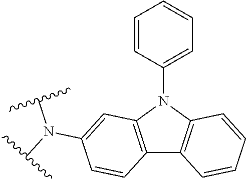 [SMILES: *N(*)c1ccc2c3ccccc3n(-c3ccccc3)c2c1] | 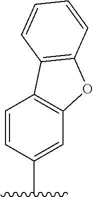 [SMILES: *c1ccc2c(c1)oc1ccccc12] |
| G37 | 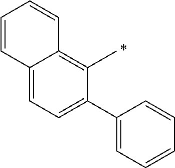 [SMILES: Cc1c(-c2ccccc2)ccc2ccccc12] | 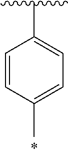 [SMILES: *c1ccc(C)cc1] | 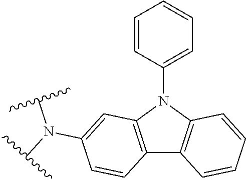 [SMILES: *N(*)c1ccc2c3ccccc3n(-c3ccccc3)c2c1] | 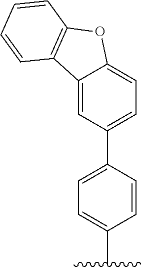 [SMILES: *c1ccc(-c2ccc3oc4ccccc4c3c2)cc1] |
| G38 | 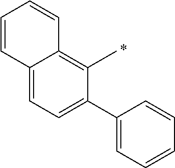 [SMILES: Cc1c(-c2ccccc2)ccc2ccccc12] | 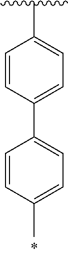 [SMILES: *c1ccc(-c2ccc(C)cc2)cc1] | 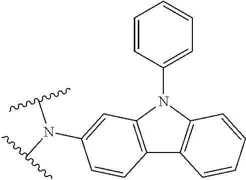 [SMILES: *N(*)c1ccc2c3ccccc3n(-c3ccccc3)c2c1] | 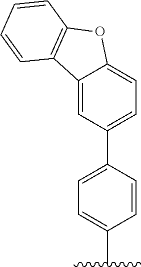 [SMILES: *c1ccc(-c2ccc3oc4ccccc4c3c2)cc1] |
| G39 | 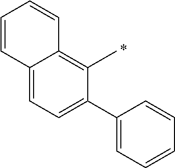 [SMILES: Cc1c(-c2ccccc2)ccc2ccccc12] | 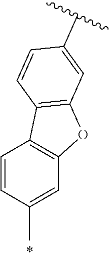 [SMILES: *c1ccc2c(c1)oc1cc(C)ccc12] | 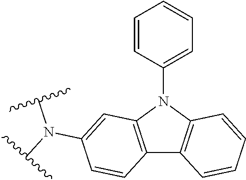 [SMILES: *N(*)c1ccc2c3ccccc3n(-c3ccccc3)c2c1] | 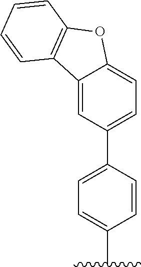 [SMILES: *c1ccc(-c2ccc3oc4ccccc4c3c2)cc1] |
| H1 | 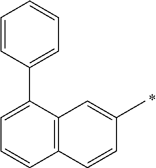 [SMILES: Cc1ccc2cccc(-c3ccccc3)c2c1] | 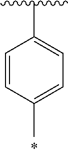 [SMILES: *c1ccc(C)cc1] | 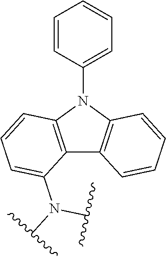 [SMILES: *N(*)c1cccc2c1c1ccccc1n2-c1ccccc1] | 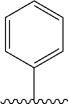 [SMILES: *c1ccccc1] |
| H2 | 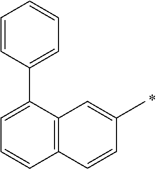 [SMILES: Cc1ccc2cccc(-c3ccccc3)c2c1] | 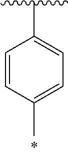 [SMILES: *c1ccc(C)cc1] | 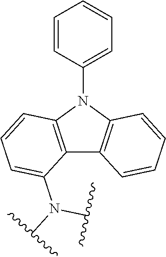 [SMILES: *N(*)c1cccc2c1c1ccccc1n2-c1ccccc1] | 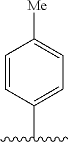 [SMILES: *c1ccc(C)cc1] |
| H3 | 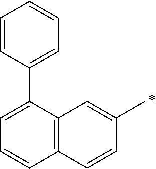 [SMILES: Cc1ccc2cccc(-c3ccccc3)c2c1] | 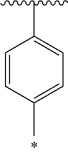 [SMILES: *c1ccc(C)cc1] | 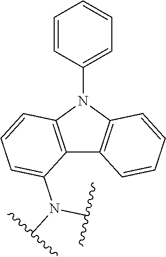 [SMILES: *N(*)c1cccc2c1c1ccccc1n2-c1ccccc1] | 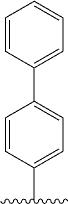 [SMILES: *c1ccc(-c2ccccc2)cc1] |
| H4 | 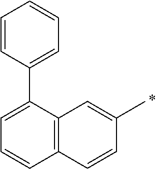 [SMILES: Cc1ccc2cccc(-c3ccccc3)c2c1] | 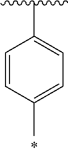 [SMILES: *c1ccc(C)cc1] | 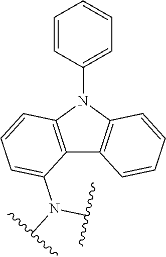 [SMILES: *N(*)c1cccc2c1c1ccccc1n2-c1ccccc1] | 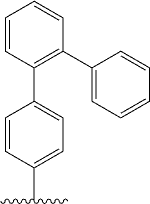 [SMILES: *c1ccc(-c2ccccc2-c2ccccc2)cc1] |
| H5 | 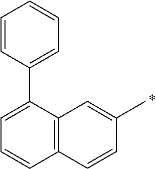 [SMILES: Cc1ccc2cccc(-c3ccccc3)c2c1] | 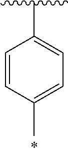 [SMILES: *c1ccc(C)cc1] | 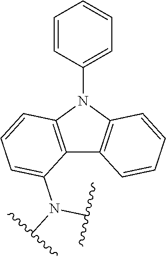 [SMILES: *N(*)c1cccc2c1c1ccccc1n2-c1ccccc1] | 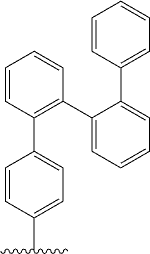 [SMILES: *c1ccc(-c2ccccc2-c2ccccc2-c2ccccc2)cc1] |
| H11 | 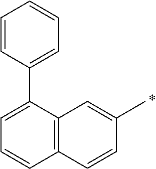 [SMILES: Cc1ccc2cccc(-c3ccccc3)c2c1] | 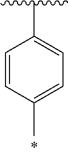 [SMILES: *c1ccc(C)cc1] | 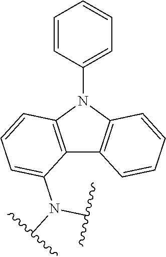 [SMILES: *N(*)c1cccc2c1c1ccccc1n2-c1ccccc1] | 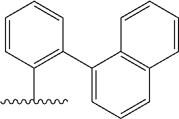 [SMILES: *c1ccccc1-c1cccc2ccccc12] |
| H12 | 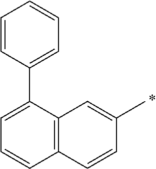 [SMILES: Cc1ccc2cccc(-c3ccccc3)c2c1] | 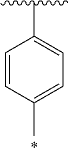 [SMILES: *c1ccc(C)cc1] | 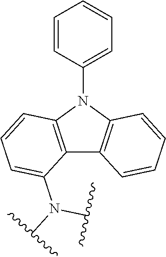 [SMILES: *N(*)c1cccc2c1c1ccccc1n2-c1ccccc1] | 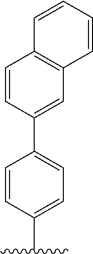 [SMILES: *c1ccc(-c2ccc3ccccc3c2)cc1] |
| H13 | 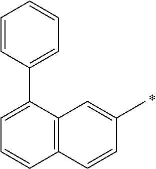 [SMILES: Cc1ccc2cccc(-c3ccccc3)c2c1] | 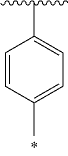 [SMILES: *c1ccc(C)cc1] | 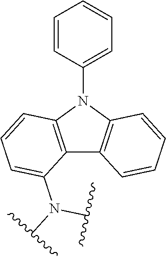 [SMILES: *N(*)c1cccc2c1c1ccccc1n2-c1ccccc1] | 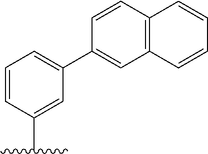 [SMILES: *c1cccc(-c2ccc3ccccc3c2)c1] |
| H14 | 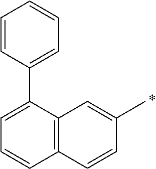 [SMILES: Cc1ccc2cccc(-c3ccccc3)c2c1] | 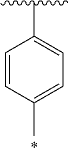 [SMILES: *c1ccc(C)cc1] | 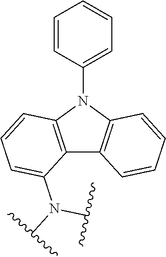 [SMILES: *N(*)c1cccc2c1c1ccccc1n2-c1ccccc1] | 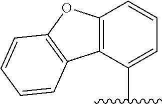 [SMILES: *c1cccc2oc3ccccc3c12] |
| H15 | 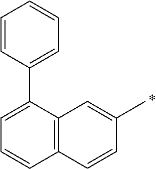 [SMILES: Cc1ccc2cccc(-c3ccccc3)c2c1] | 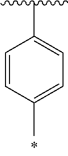 [SMILES: *c1ccc(C)cc1] | 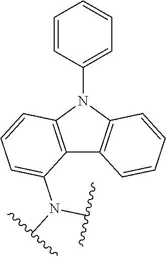 [SMILES: *N(*)c1cccc2c1c1ccccc1n2-c1ccccc1] | 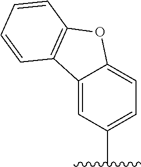 [SMILES: *c1ccc2oc3ccccc3c2c1] |
| H16 | 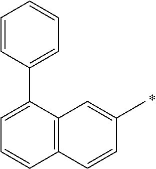 [SMILES: Cc1ccc2cccc(-c3ccccc3)c2c1] | 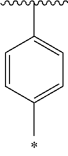 [SMILES: *c1ccc(C)cc1] | 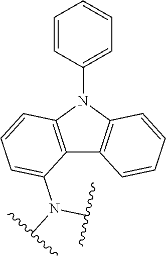 [SMILES: *N(*)c1cccc2c1c1ccccc1n2-c1ccccc1] | 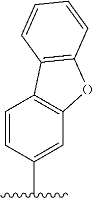 [SMILES: *c1ccc2c(c1)oc1ccccc12] |
| H17 | 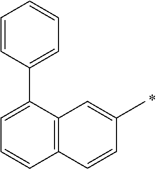 [SMILES: Cc1ccc2cccc(-c3ccccc3)c2c1] | 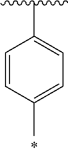 [SMILES: *c1ccc(C)cc1] | 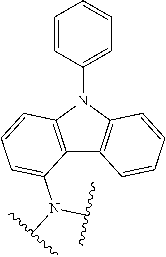 [SMILES: *N(*)c1cccc2c1c1ccccc1n2-c1ccccc1] | 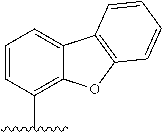 [SMILES: *c1cccc2c1oc1ccccc12] |
| H18 | 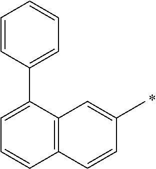 [SMILES: Cc1ccc2cccc(-c3ccccc3)c2c1] | 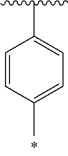 [SMILES: *c1ccc(C)cc1] | 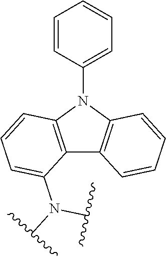 [SMILES: *N(*)c1cccc2c1c1ccccc1n2-c1ccccc1] | 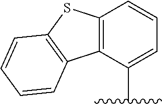 [SMILES: *c1cccc2sc3ccccc3c12] |
| H19 | 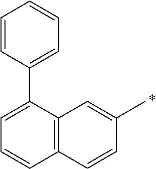 [SMILES: Cc1ccc2cccc(-c3ccccc3)c2c1] | 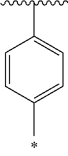 [SMILES: *c1ccc(C)cc1] | 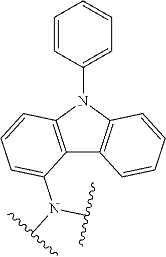 [SMILES: *N(*)c1cccc2c1c1ccccc1n2-c1ccccc1] | 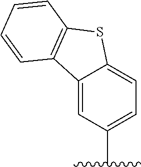 [SMILES: *c1ccc2sc3ccccc3c2c1] |
| H20 | 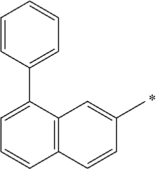 [SMILES: Cc1ccc2cccc(-c3ccccc3)c2c1] | 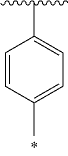 [SMILES: *c1ccc(C)cc1] | 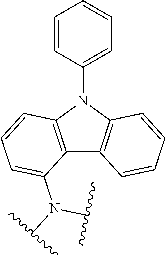 [SMILES: *N(*)c1cccc2c1c1ccccc1n2-c1ccccc1] | 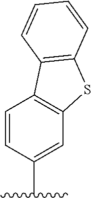 [SMILES: *c1ccc2c(c1)sc1ccccc12] |
| H21 | 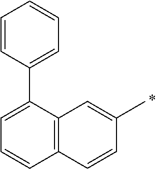 [SMILES: Cc1ccc2cccc(-c3ccccc3)c2c1] | 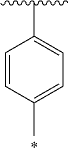 [SMILES: *c1ccc(C)cc1] | 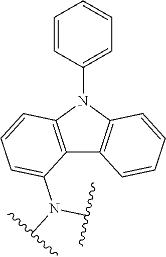 [SMILES: *N(*)c1cccc2c1c1ccccc1n2-c1ccccc1] | 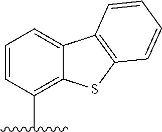 [SMILES: *c1cccc2c1sc1ccccc12] |
| H22 | 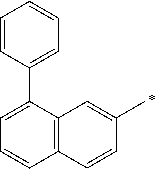 [SMILES: Cc1ccc2cccc(-c3ccccc3)c2c1] | 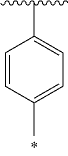 [SMILES: *c1ccc(C)cc1] | 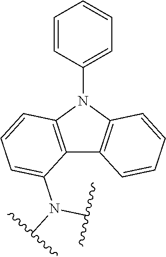 [SMILES: *N(*)c1cccc2c1c1ccccc1n2-c1ccccc1] | 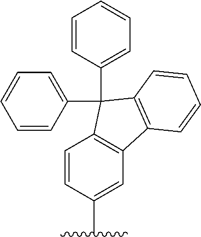 [SMILES: *c1ccc2c(c1)-c1ccccc1C2(c1ccccc1)c1ccccc1] |
| H23 | 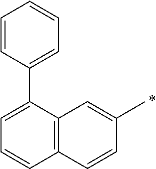 [SMILES: Cc1ccc2cccc(-c3ccccc3)c2c1] | 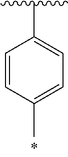 [SMILES: *c1ccc(C)cc1] | 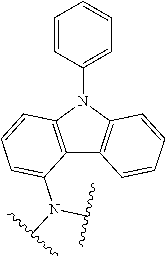 [SMILES: *N(*)c1cccc2c1c1ccccc1n2-c1ccccc1] | 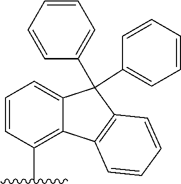 [SMILES: *c1cccc2c1-c1ccccc1C2(c1ccccc1)c1ccccc1] |
| H24 | 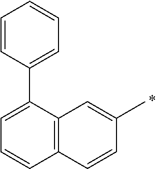 [SMILES: Cc1ccc2cccc(-c3ccccc3)c2c1] | 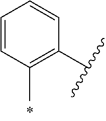 [SMILES: *c1ccccc1C] | 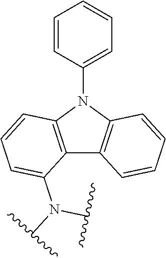 [SMILES: *N(*)c1cccc2c1c1ccccc1n2-c1ccccc1] | 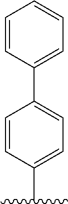 [SMILES: *c1ccc(-c2ccccc2)cc1] |
| H25 | 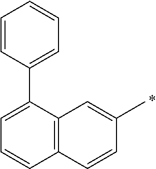 [SMILES: Cc1ccc2cccc(-c3ccccc3)c2c1] | 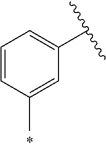 [SMILES: *c1cccc(C)c1] | 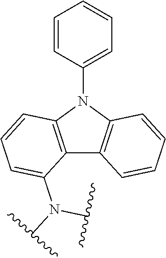 [SMILES: *N(*)c1cccc2c1c1ccccc1n2-c1ccccc1] | 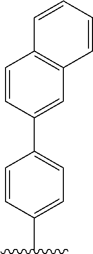 [SMILES: *c1ccc(-c2ccc3ccccc3c2)cc1] |
| H26 | 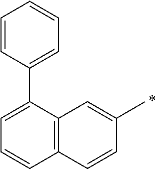 [SMILES: Cc1ccc2cccc(-c3ccccc3)c2c1] | 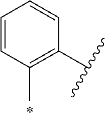 [SMILES: *c1ccccc1C] | 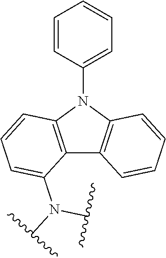 [SMILES: *N(*)c1cccc2c1c1ccccc1n2-c1ccccc1] | 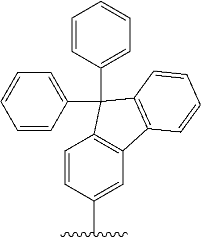 [SMILES: *c1ccc2c(c1)-c1ccccc1C2(c1ccccc1)c1ccccc1] |
| H27 | 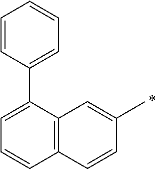 [SMILES: Cc1ccc2cccc(-c3ccccc3)c2c1] | 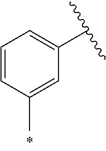 [SMILES: *c1cccc(C)c1] | 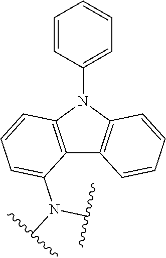 [SMILES: *N(*)c1cccc2c1c1ccccc1n2-c1ccccc1] | 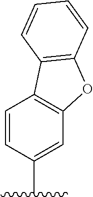 [SMILES: *c1ccc2c(c1)oc1ccccc12] |
| H28 | 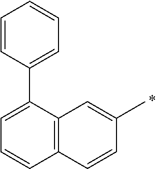 [SMILES: Cc1ccc2cccc(-c3ccccc3)c2c1] | 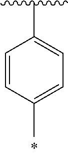 [SMILES: *c1ccc(C)cc1] | 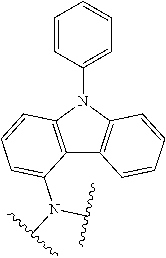 [SMILES: *N(*)c1cccc2c1c1ccccc1n2-c1ccccc1] | 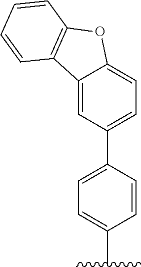 [SMILES: *c1ccc(-c2ccc3oc4ccccc4c3c2)cc1] |
| H29 | 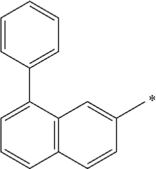 [SMILES: Cc1ccc2cccc(-c3ccccc3)c2c1] | 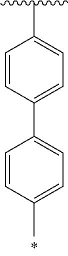 [SMILES: *c1ccc(-c2ccc(C)cc2)cc1] | 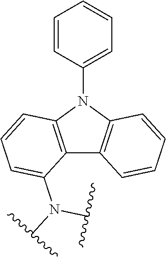 [SMILES: *N(*)c1cccc2c1c1ccccc1n2-c1ccccc1] | 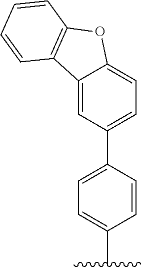 [SMILES: *c1ccc(-c2ccc3oc4ccccc4c3c2)cc1] |
| H30 | 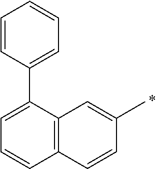 [SMILES: Cc1ccc2cccc(-c3ccccc3)c2c1] | 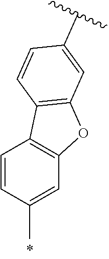 [SMILES: *c1ccc2c(c1)oc1cc(C)ccc12] | 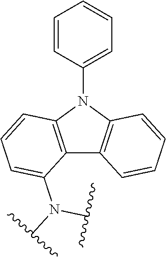 [SMILES: *N(*)c1cccc2c1c1ccccc1n2-c1ccccc1] | 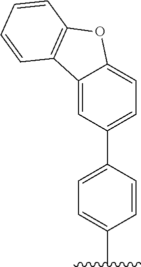 [SMILES: *c1ccc(-c2ccc3oc4ccccc4c3c2)cc1] |
| H31 | 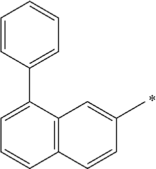 [SMILES: Cc1ccc2cccc(-c3ccccc3)c2c1] | 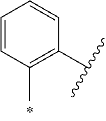 [SMILES: *c1ccccc1C] | 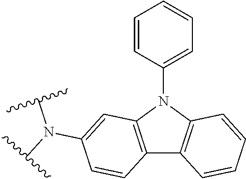 [SMILES: *N(*)c1ccc2c3ccccc3n(-c3ccccc3)c2c1] | 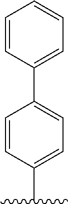 [SMILES: *c1ccc(-c2ccccc2)cc1] |
| H32 | 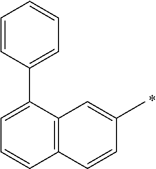 [SMILES: Cc1ccc2cccc(-c3ccccc3)c2c1] | 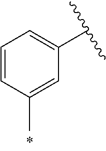 [SMILES: *c1cccc(C)c1] | 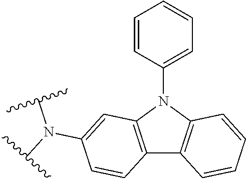 [SMILES: *N(*)c1ccc2c3ccccc3n(-c3ccccc3)c2c1] | 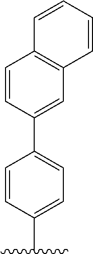 [SMILES: *c1ccc(-c2ccc3ccccc3c2)cc1] |
| H33 | 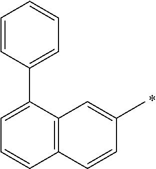 [SMILES: Cc1ccc2cccc(-c3ccccc3)c2c1] | 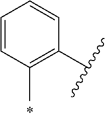 [SMILES: *c1ccccc1C] | 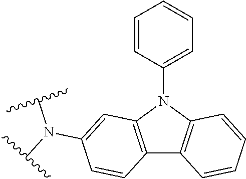 [SMILES: *N(*)c1ccc2c3ccccc3n(-c3ccccc3)c2c1] | 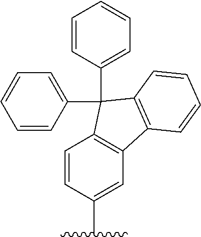 [SMILES: *c1ccc2c(c1)-c1ccccc1C2(c1ccccc1)c1ccccc1] |
| H34 | 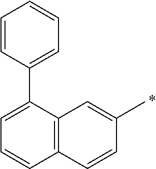 [SMILES: Cc1ccc2cccc(-c3ccccc3)c2c1] | 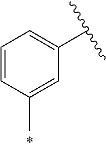 [SMILES: *c1cccc(C)c1] | 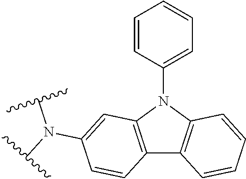 [SMILES: *N(*)c1ccc2c3ccccc3n(-c3ccccc3)c2c1] | 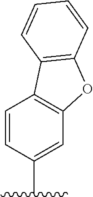 [SMILES: *c1ccc2c(c1)oc1ccccc12] |
| H35 | 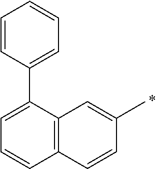 [SMILES: Cc1ccc2cccc(-c3ccccc3)c2c1] | 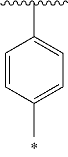 [SMILES: *c1ccc(C)cc1] | 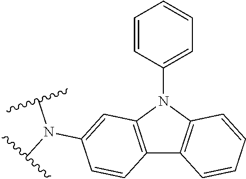 [SMILES: *N(*)c1ccc2c3ccccc3n(-c3ccccc3)c2c1] | 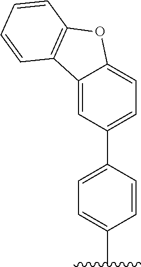 [SMILES: *c1ccc(-c2ccc3oc4ccccc4c3c2)cc1] |
| H36 | 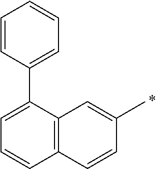 [SMILES: Cc1ccc2cccc(-c3ccccc3)c2c1] | 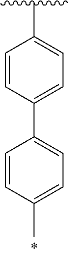 [SMILES: *c1ccc(-c2ccc(C)cc2)cc1] | 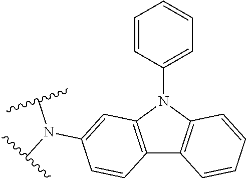 [SMILES: *N(*)c1ccc2c3ccccc3n(-c3ccccc3)c2c1] | 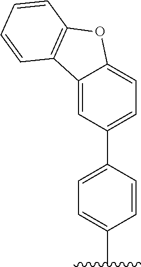 [SMILES: *c1ccc(-c2ccc3oc4ccccc4c3c2)cc1] |
| H37 | 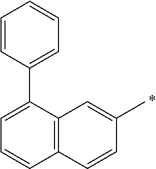 [SMILES: Cc1ccc2cccc(-c3ccccc3)c2c1] | 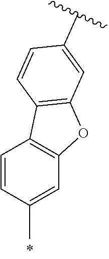 [SMILES: *c1ccc2c(c1)oc1cc(C)ccc12] | 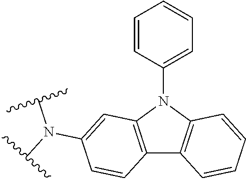 [SMILES: *N(*)c1ccc2c3ccccc3n(-c3ccccc3)c2c1] | 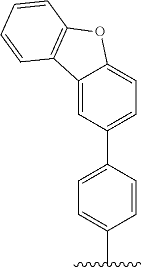 [SMILES: *c1ccc(-c2ccc3oc4ccccc4c3c2)cc1] |
| I1 | 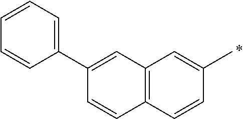 [SMILES: Cc1ccc2ccc(-c3ccccc3)cc2c1] | 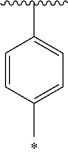 [SMILES: *c1ccc(C)cc1] | 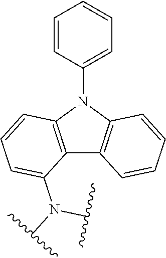 [SMILES: *N(*)c1cccc2c1c1ccccc1n2-c1ccccc1] | 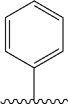 [SMILES: *c1ccccc1] |
| I2 | 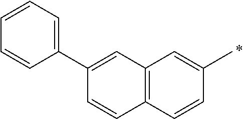 [SMILES: Cc1ccc2ccc(-c3ccccc3)cc2c1] | 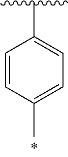 [SMILES: *c1ccc(C)cc1] | 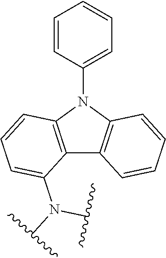 [SMILES: *N(*)c1cccc2c1c1ccccc1n2-c1ccccc1] | 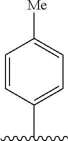 [SMILES: *c1ccc(C)cc1] |
| I3 | 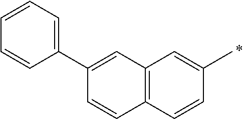 [SMILES: Cc1ccc2ccc(-c3ccccc3)cc2c1] | 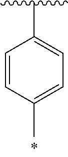 [SMILES: *c1ccc(C)cc1] | 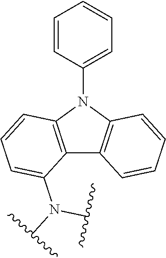 [SMILES: *N(*)c1cccc2c1c1ccccc1n2-c1ccccc1] | 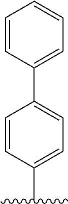 [SMILES: *c1ccc(-c2ccccc2)cc1] |
| I4 | 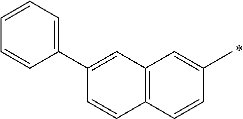 [SMILES: Cc1ccc2ccc(-c3ccccc3)cc2c1] | 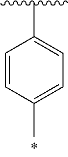 [SMILES: *c1ccc(C)cc1] | 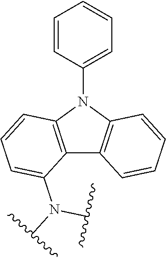 [SMILES: *N(*)c1cccc2c1c1ccccc1n2-c1ccccc1] | 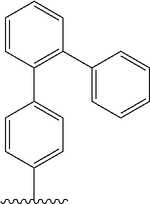 [SMILES: *c1ccc(-c2ccccc2-c2ccccc2)cc1] |
| I5 | 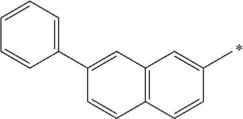 [SMILES: Cc1ccc2ccc(-c3ccccc3)cc2c1] | 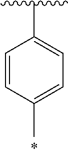 [SMILES: *c1ccc(C)cc1] | 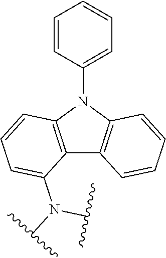 [SMILES: *N(*)c1cccc2c1c1ccccc1n2-c1ccccc1] | 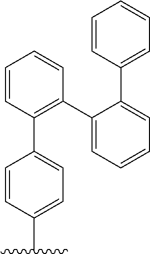 [SMILES: *c1ccc(-c2ccccc2-c2ccccc2-c2ccccc2)cc1] |
| I6 | 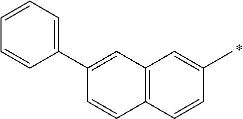 [SMILES: Cc1ccc2ccc(-c3ccccc3)cc2c1] | 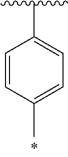 [SMILES: *c1ccc(C)cc1] | 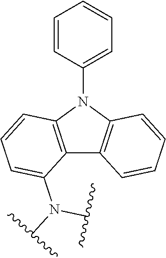 [SMILES: *N(*)c1cccc2c1c1ccccc1n2-c1ccccc1] | 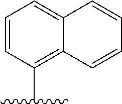 [SMILES: *c1cccc2ccccc12] |
| I7 | 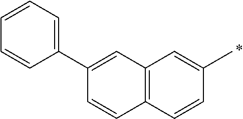 [SMILES: Cc1ccc2ccc(-c3ccccc3)cc2c1] | 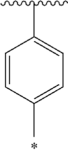 [SMILES: *c1ccc(C)cc1] | 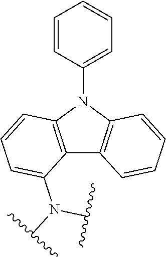 [SMILES: *N(*)c1cccc2c1c1ccccc1n2-c1ccccc1] | 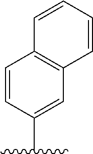 [SMILES: *c1ccc2ccccc2c1] |
| I8 | 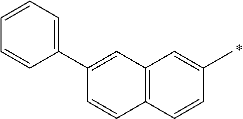 [SMILES: Cc1ccc2ccc(-c3ccccc3)cc2c1] | 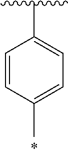 [SMILES: *c1ccc(C)cc1] | 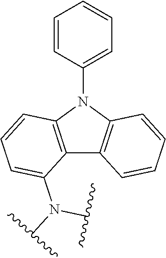 [SMILES: *N(*)c1cccc2c1c1ccccc1n2-c1ccccc1] | 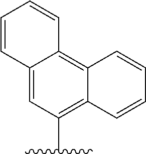 [SMILES: *c1cc2ccccc2c2ccccc12] |
| I9 | 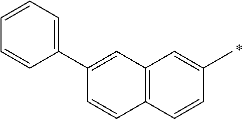 [SMILES: Cc1ccc2ccc(-c3ccccc3)cc2c1] | 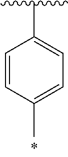 [SMILES: *c1ccc(C)cc1] | 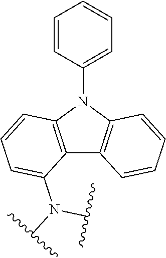 [SMILES: *N(*)c1cccc2c1c1ccccc1n2-c1ccccc1] | 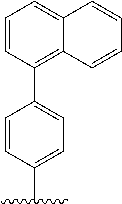 [SMILES: *c1ccc(-c2cccc3ccccc23)cc1] |
| I10 | 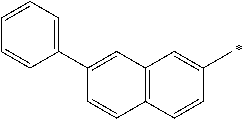 [SMILES: Cc1ccc2ccc(-c3ccccc3)cc2c1] | 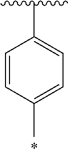 [SMILES: *c1ccc(C)cc1] | 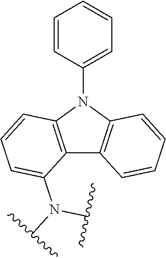 [SMILES: *N(*)c1cccc2c1c1ccccc1n2-c1ccccc1] | 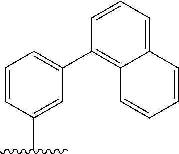 [SMILES: *c1cccc(-c2cccc3ccccc23)c1] |
| I11 | 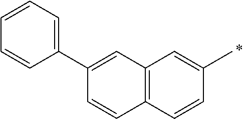 [SMILES: Cc1ccc2ccc(-c3ccccc3)cc2c1] | 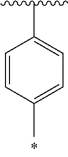 [SMILES: *c1ccc(C)cc1] | 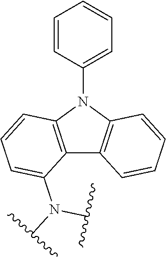 [SMILES: *N(*)c1cccc2c1c1ccccc1n2-c1ccccc1] | 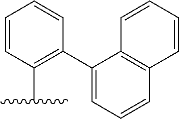 [SMILES: *c1ccccc1-c1cccc2ccccc12] |
| I12 | 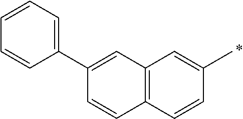 [SMILES: Cc1ccc2ccc(-c3ccccc3)cc2c1] | 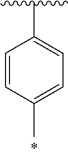 [SMILES: *c1ccc(C)cc1] | 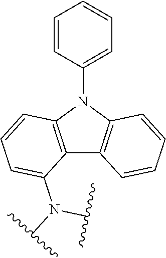 [SMILES: *N(*)c1cccc2c1c1ccccc1n2-c1ccccc1] | 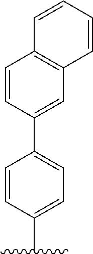 [SMILES: *c1ccc(-c2ccc3ccccc3c2)cc1] |
| I13 | 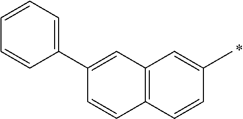 [SMILES: Cc1ccc2ccc(-c3ccccc3)cc2c1] | 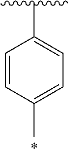 [SMILES: *c1ccc(C)cc1] | 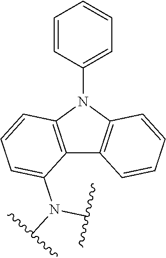 [SMILES: *N(*)c1cccc2c1c1ccccc1n2-c1ccccc1] | 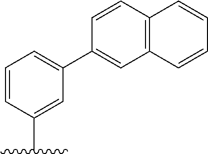 [SMILES: *c1cccc(-c2ccc3ccccc3c2)c1] |
| I14 | 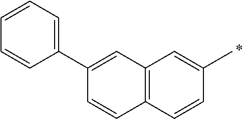 [SMILES: Cc1ccc2ccc(-c3ccccc3)cc2c1] | 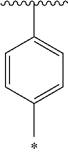 [SMILES: *c1ccc(C)cc1] | 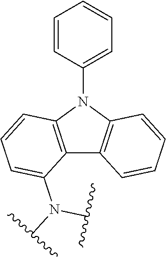 [SMILES: *N(*)c1cccc2c1c1ccccc1n2-c1ccccc1] | 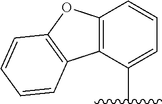 [SMILES: *c1cccc2oc3ccccc3c12] |
| I15 | 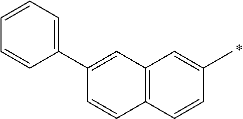 [SMILES: Cc1ccc2ccc(-c3ccccc3)cc2c1] | 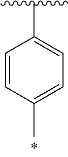 [SMILES: *c1ccc(C)cc1] | 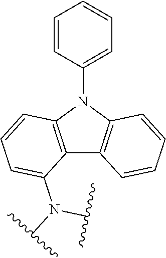 [SMILES: *N(*)c1cccc2c1c1ccccc1n2-c1ccccc1] | 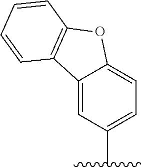 [SMILES: *c1ccc2oc3ccccc3c2c1] |
| I16 | 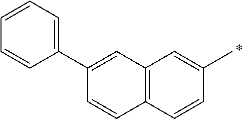 [SMILES: Cc1ccc2ccc(-c3ccccc3)cc2c1] | 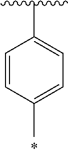 [SMILES: *c1ccc(C)cc1] | 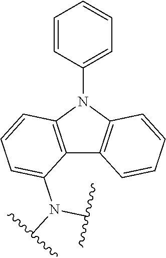 [SMILES: *N(*)c1cccc2c1c1ccccc1n2-c1ccccc1] | 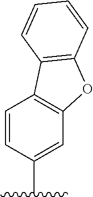 [SMILES: *c1ccc2c(c1)oc1ccccc12] |
| I17 | 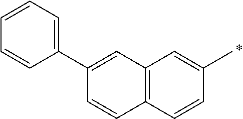 [SMILES: Cc1ccc2ccc(-c3ccccc3)cc2c1] | 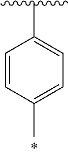 [SMILES: *c1ccc(C)cc1] | 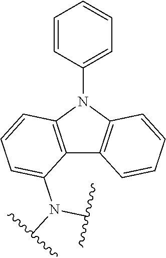 [SMILES: *N(*)c1cccc2c1c1ccccc1n2-c1ccccc1] | 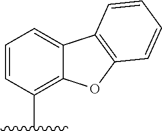 [SMILES: *c1cccc2c1oc1ccccc12] |
| I18 | 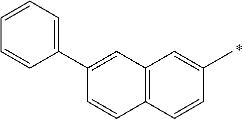 [SMILES: Cc1ccc2ccc(-c3ccccc3)cc2c1] | 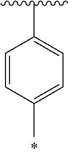 [SMILES: *c1ccc(C)cc1] | 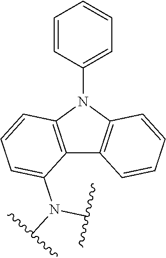 [SMILES: *N(*)c1cccc2c1c1ccccc1n2-c1ccccc1] | 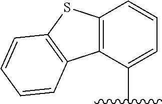 [SMILES: *c1cccc2sc3ccccc3c12] |
| I19 | 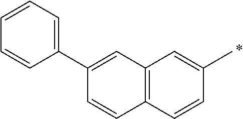 [SMILES: Cc1ccc2ccc(-c3ccccc3)cc2c1] | 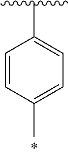 [SMILES: *c1ccc(C)cc1] | 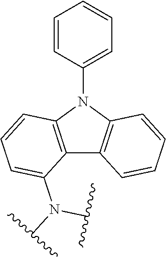 [SMILES: *N(*)c1cccc2c1c1ccccc1n2-c1ccccc1] | 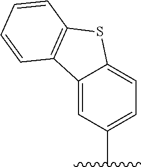 [SMILES: *c1ccc2sc3ccccc3c2c1] |
| I20 | 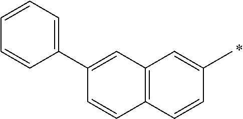 [SMILES: Cc1ccc2ccc(-c3ccccc3)cc2c1] | 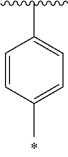 [SMILES: *c1ccc(C)cc1] | 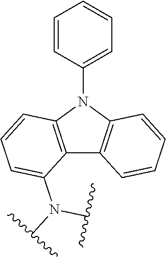 [SMILES: *N(*)c1cccc2c1c1ccccc1n2-c1ccccc1] | 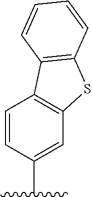 [SMILES: *c1ccc2c(c1)sc1ccccc12] |
| I21 | 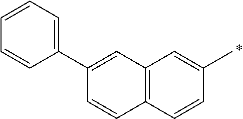 [SMILES: Cc1ccc2ccc(-c3ccccc3)cc2c1] | 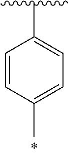 [SMILES: *c1ccc(C)cc1] | 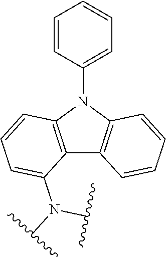 [SMILES: *N(*)c1cccc2c1c1ccccc1n2-c1ccccc1] | 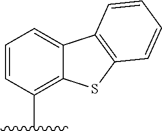 [SMILES: *c1cccc2c1sc1ccccc12] |
| I22 | 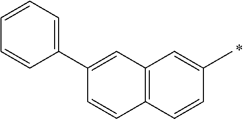 [SMILES: Cc1ccc2ccc(-c3ccccc3)cc2c1] | 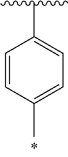 [SMILES: *c1ccc(C)cc1] | 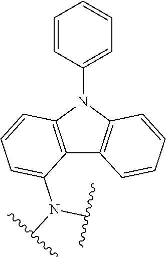 [SMILES: *N(*)c1cccc2c1c1ccccc1n2-c1ccccc1] | 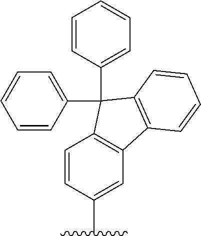 [SMILES: *c1ccc2c(c1)-c1ccccc1C2(c1ccccc1)c1ccccc1] |
| I23 | 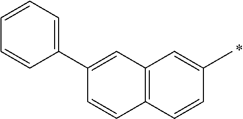 [SMILES: Cc1ccc2ccc(-c3ccccc3)cc2c1] | 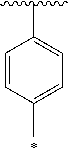 [SMILES: *c1ccc(C)cc1] | 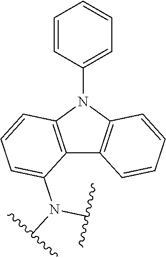 [SMILES: *N(*)c1cccc2c1c1ccccc1n2-c1ccccc1] | 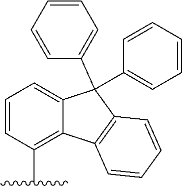 [SMILES: *c1cccc2c1-c1ccccc1C2(c1ccccc1)c1ccccc1] |
| I24 | 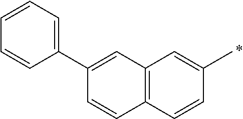 [SMILES: Cc1ccc2ccc(-c3ccccc3)cc2c1] | 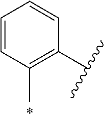 [SMILES: *c1ccccc1C] | 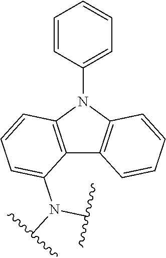 [SMILES: *N(*)c1cccc2c1c1ccccc1n2-c1ccccc1] | 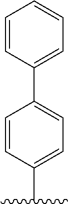 [SMILES: *c1ccc(-c2ccccc2)cc1] |
| I25 | 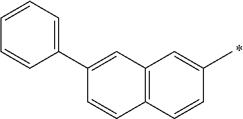 [SMILES: Cc1ccc2ccc(-c3ccccc3)cc2c1] | 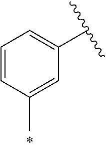 [SMILES: *c1cccc(C)c1] | 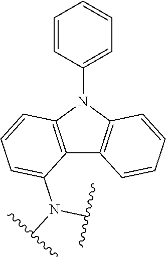 [SMILES: *N(*)c1cccc2c1c1ccccc1n2-c1ccccc1] | 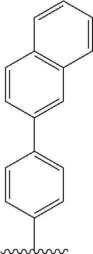 [SMILES: *c1ccc(-c2ccc3ccccc3c2)cc1] |
| I26 | 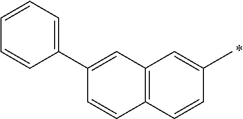 [SMILES: Cc1ccc2ccc(-c3ccccc3)cc2c1] | 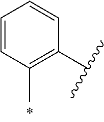 [SMILES: *c1ccccc1C] | 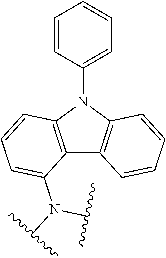 [SMILES: *N(*)c1cccc2c1c1ccccc1n2-c1ccccc1] | 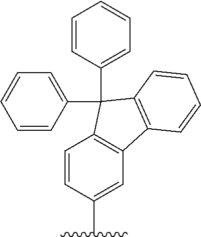 [SMILES: *c1ccc2c(c1)-c1ccccc1C2(c1ccccc1)c1ccccc1] |
| I27 | 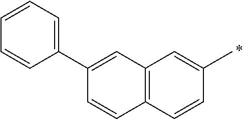 [SMILES: Cc1ccc2ccc(-c3ccccc3)cc2c1] | 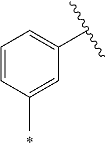 [SMILES: *c1cccc(C)c1] | 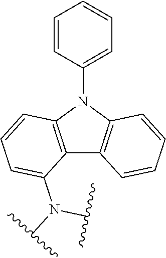 [SMILES: *N(*)c1cccc2c1c1ccccc1n2-c1ccccc1] | 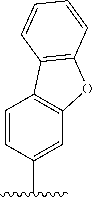 [SMILES: *c1ccc2c(c1)oc1ccccc12] |
| I28 | 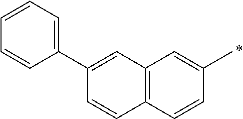 [SMILES: Cc1ccc2ccc(-c3ccccc3)cc2c1] | 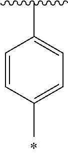 [SMILES: *c1ccc(C)cc1] | 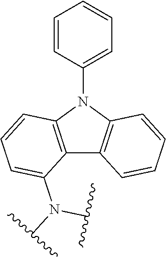 [SMILES: *N(*)c1cccc2c1c1ccccc1n2-c1ccccc1] | 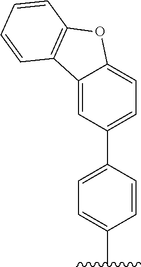 [SMILES: *c1ccc(-c2ccc3oc4ccccc4c3c2)cc1] |
| I29 | 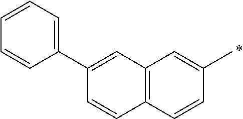 [SMILES: Cc1ccc2ccc(-c3ccccc3)cc2c1] | 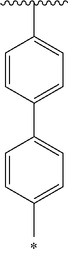 [SMILES: *c1ccc(-c2ccc(C)cc2)cc1] | 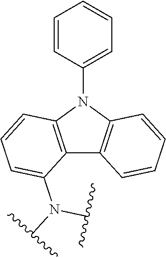 [SMILES: *N(*)c1cccc2c1c1ccccc1n2-c1ccccc1] | 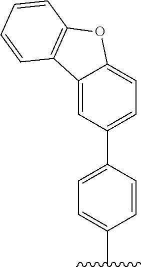 [SMILES: *c1ccc(-c2ccc3oc4ccccc4c3c2)cc1] |
| I30 | 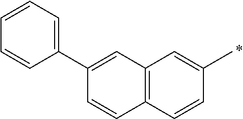 [SMILES: Cc1ccc2ccc(-c3ccccc3)cc2c1] | 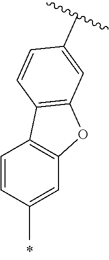 [SMILES: *c1ccc2c(c1)oc1cc(C)ccc12] | 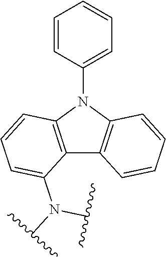 [SMILES: *N(*)c1cccc2c1c1ccccc1n2-c1ccccc1] | 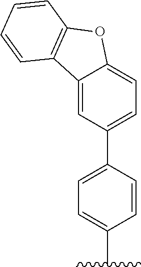 [SMILES: *c1ccc(-c2ccc3oc4ccccc4c3c2)cc1] |
| I31 | 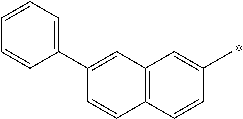 [SMILES: Cc1ccc2ccc(-c3ccccc3)cc2c1] | 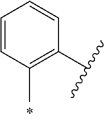 [SMILES: *c1ccccc1C] | 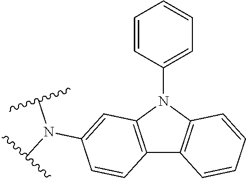 [SMILES: *N(*)c1ccc2c3ccccc3n(-c3ccccc3)c2c1] | 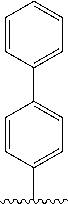 [SMILES: *c1ccc(-c2ccccc2)cc1] |
| I32 | 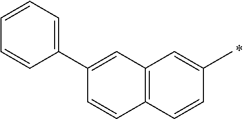 [SMILES: Cc1ccc2ccc(-c3ccccc3)cc2c1] | 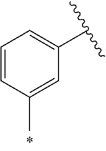 [SMILES: *c1cccc(C)c1] | 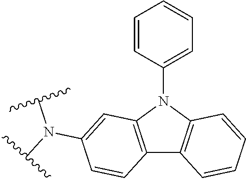 [SMILES: *N(*)c1ccc2c3ccccc3n(-c3ccccc3)c2c1] | 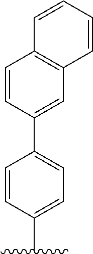 [SMILES: *c1ccc(-c2ccc3ccccc3c2)cc1] |
| I33 | 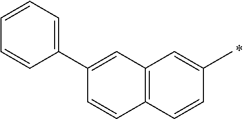 [SMILES: Cc1ccc2ccc(-c3ccccc3)cc2c1] | 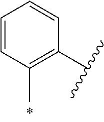 [SMILES: *c1ccccc1C] | 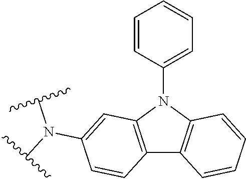 [SMILES: *N(*)c1ccc2c3ccccc3n(-c3ccccc3)c2c1] | 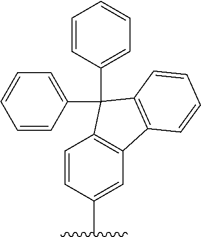 [SMILES: *c1ccc2c(c1)-c1ccccc1C2(c1ccccc1)c1ccccc1] |
| I34 | 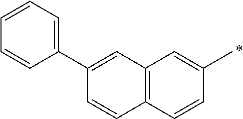 [SMILES: Cc1ccc2ccc(-c3ccccc3)cc2c1] | 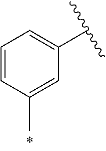 [SMILES: *c1cccc(C)c1] | 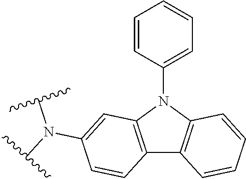 [SMILES: *N(*)c1ccc2c3ccccc3n(-c3ccccc3)c2c1] | 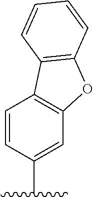 [SMILES: *c1ccc2c(c1)oc1ccccc12] |
| I35 | 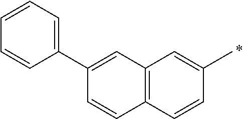 [SMILES: Cc1ccc2ccc(-c3ccccc3)cc2c1] | 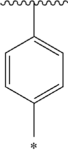 [SMILES: *c1ccc(C)cc1] | 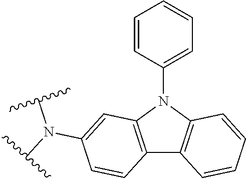 [SMILES: *N(*)c1ccc2c3ccccc3n(-c3ccccc3)c2c1] | 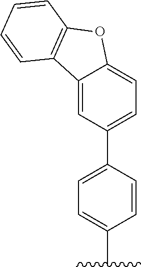 [SMILES: *c1ccc(-c2ccc3oc4ccccc4c3c2)cc1] |
| I36 | 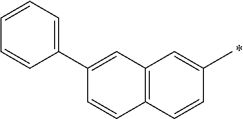 [SMILES: Cc1ccc2ccc(-c3ccccc3)cc2c1] | 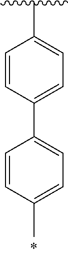 [SMILES: *c1ccc(-c2ccc(C)cc2)cc1] | 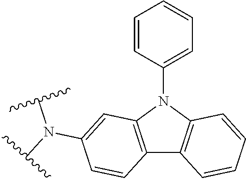 [SMILES: *N(*)c1ccc2c3ccccc3n(-c3ccccc3)c2c1] | 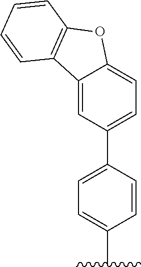 [SMILES: *c1ccc(-c2ccc3oc4ccccc4c3c2)cc1] |
| I37 | 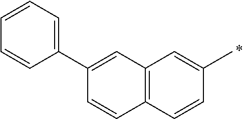 [SMILES: Cc1ccc2ccc(-c3ccccc3)cc2c1] | 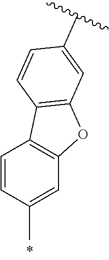 [SMILES: *c1ccc2c(c1)oc1cc(C)ccc12] | 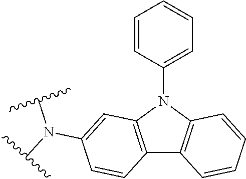 [SMILES: *N(*)c1ccc2c3ccccc3n(-c3ccccc3)c2c1] | 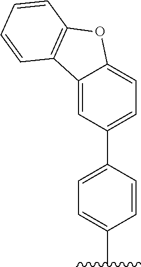 [SMILES: *c1ccc(-c2ccc3oc4ccccc4c3c2)cc1] |
| J1 | 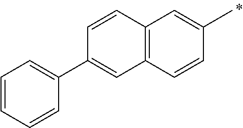 [SMILES: Cc1ccc2cc(-c3ccccc3)ccc2c1] | 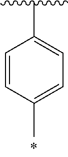 [SMILES: *c1ccc(C)cc1] | 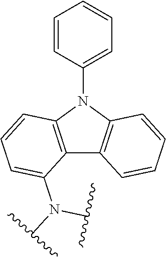 [SMILES: *N(*)c1cccc2c1c1ccccc1n2-c1ccccc1] | 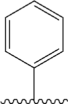 [SMILES: *c1ccccc1] |
| J2 | 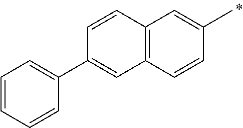 [SMILES: Cc1ccc2cc(-c3ccccc3)ccc2c1] | 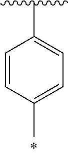 [SMILES: *c1ccc(C)cc1] | 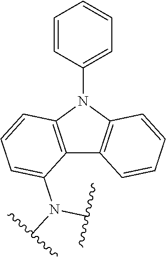 [SMILES: *N(*)c1cccc2c1c1ccccc1n2-c1ccccc1] | 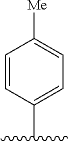 [SMILES: *c1ccc(C)cc1] |
| J3 | 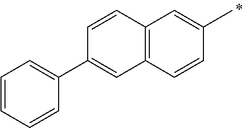 [SMILES: Cc1ccc2cc(-c3ccccc3)ccc2c1] | 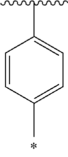 [SMILES: *c1ccc(C)cc1] | 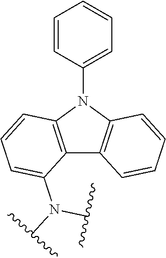 [SMILES: *N(*)c1cccc2c1c1ccccc1n2-c1ccccc1] | 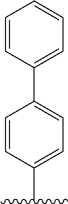 [SMILES: *c1ccc(-c2ccccc2)cc1] |
| J4 | 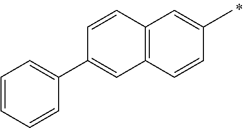 [SMILES: Cc1ccc2cc(-c3ccccc3)ccc2c1] | 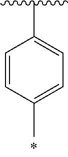 [SMILES: *c1ccc(C)cc1] | 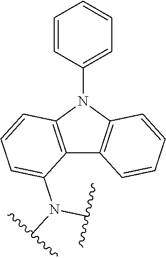 [SMILES: *N(*)c1cccc2c1c1ccccc1n2-c1ccccc1] | 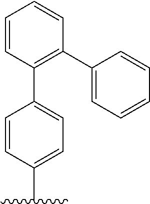 [SMILES: *c1ccc(-c2ccccc2-c2ccccc2)cc1] |
| J5 | 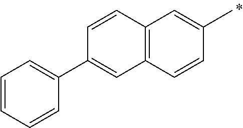 [SMILES: Cc1ccc2cc(-c3ccccc3)ccc2c1] | 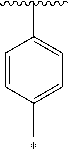 [SMILES: *c1ccc(C)cc1] | 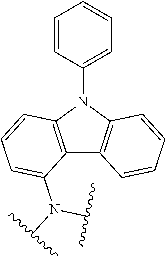 [SMILES: *N(*)c1cccc2c1c1ccccc1n2-c1ccccc1] | 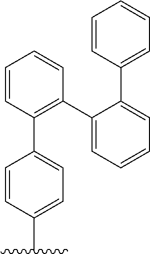 [SMILES: *c1ccc(-c2ccccc2-c2ccccc2-c2ccccc2)cc1] |
| J6 | 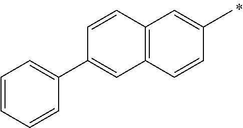 [SMILES: Cc1ccc2cc(-c3ccccc3)ccc2c1] | 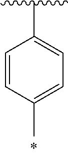 [SMILES: *c1ccc(C)cc1] | 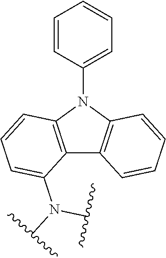 [SMILES: *N(*)c1cccc2c1c1ccccc1n2-c1ccccc1] | 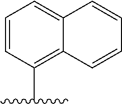 [SMILES: *c1cccc2ccccc12] |
| J7 | 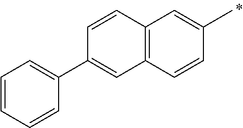 [SMILES: Cc1ccc2cc(-c3ccccc3)ccc2c1] | 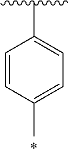 [SMILES: *c1ccc(C)cc1] | 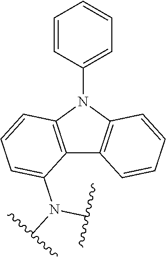 [SMILES: *N(*)c1cccc2c1c1ccccc1n2-c1ccccc1] | 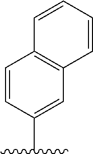 [SMILES: *c1ccc2ccccc2c1] |
| J8 | 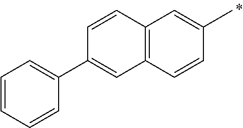 [SMILES: Cc1ccc2cc(-c3ccccc3)ccc2c1] | 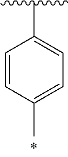 [SMILES: *c1ccc(C)cc1] | 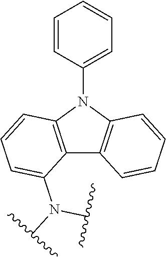 [SMILES: *N(*)c1cccc2c1c1ccccc1n2-c1ccccc1] | 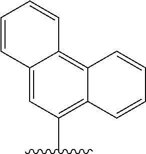 [SMILES: *c1cc2ccccc2c2ccccc12] |
| J9 | 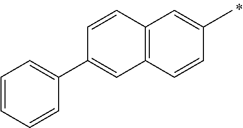 [SMILES: Cc1ccc2cc(-c3ccccc3)ccc2c1] | 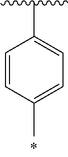 [SMILES: *c1ccc(C)cc1] | 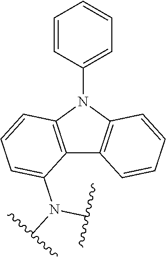 [SMILES: *N(*)c1cccc2c1c1ccccc1n2-c1ccccc1] | 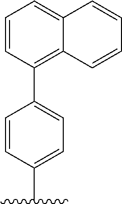 [SMILES: *c1ccc(-c2cccc3ccccc23)cc1] |
| J10 | 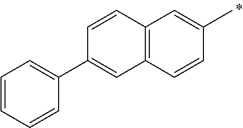 [SMILES: Cc1ccc2cc(-c3ccccc3)ccc2c1] | 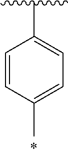 [SMILES: *c1ccc(C)cc1] | 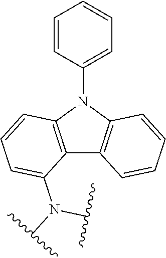 [SMILES: *N(*)c1cccc2c1c1ccccc1n2-c1ccccc1] | 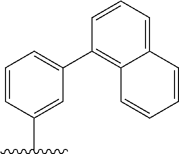 [SMILES: *c1cccc(-c2cccc3ccccc23)c1] |
| J11 | 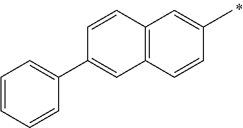 [SMILES: Cc1ccc2cc(-c3ccccc3)ccc2c1] | 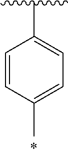 [SMILES: *c1ccc(C)cc1] | 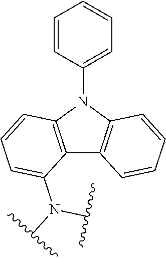 [SMILES: *N(*)c1cccc2c1c1ccccc1n2-c1ccccc1] | 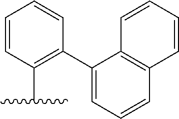 [SMILES: *c1ccccc1-c1cccc2ccccc12] |
| J12 | 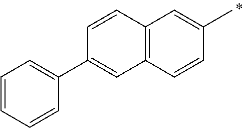 [SMILES: Cc1ccc2cc(-c3ccccc3)ccc2c1] | 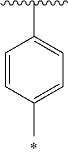 [SMILES: *c1ccc(C)cc1] | 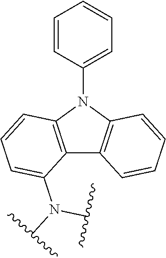 [SMILES: *N(*)c1cccc2c1c1ccccc1n2-c1ccccc1] | 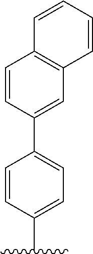 [SMILES: *c1ccc(-c2ccc3ccccc3c2)cc1] |
| J13 | 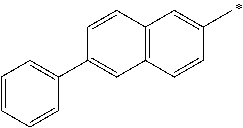 [SMILES: Cc1ccc2cc(-c3ccccc3)ccc2c1] | 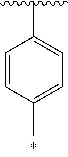 [SMILES: *c1ccc(C)cc1] | 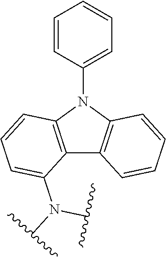 [SMILES: *N(*)c1cccc2c1c1ccccc1n2-c1ccccc1] | 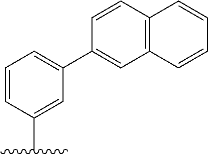 [SMILES: *c1cccc(-c2ccc3ccccc3c2)c1] |
| J14 | 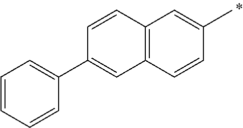 [SMILES: Cc1ccc2cc(-c3ccccc3)ccc2c1] | 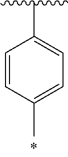 [SMILES: *c1ccc(C)cc1] | 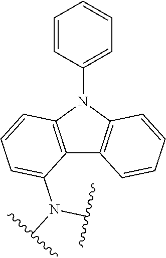 [SMILES: *N(*)c1cccc2c1c1ccccc1n2-c1ccccc1] | 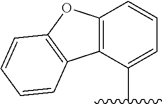 [SMILES: *c1cccc2oc3ccccc3c12] |
| J15 | 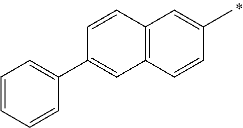 [SMILES: Cc1ccc2cc(-c3ccccc3)ccc2c1] | 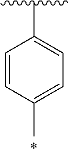 [SMILES: *c1ccc(C)cc1] | 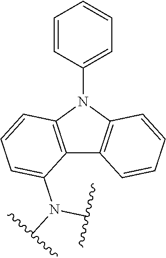 [SMILES: *N(*)c1cccc2c1c1ccccc1n2-c1ccccc1] | 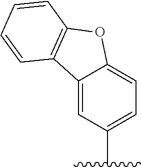 [SMILES: *c1ccc2oc3ccccc3c2c1] |
| J16 | 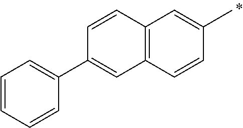 [SMILES: Cc1ccc2cc(-c3ccccc3)ccc2c1] | 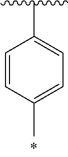 [SMILES: *c1ccc(C)cc1] | 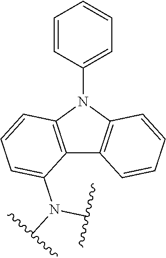 [SMILES: *N(*)c1cccc2c1c1ccccc1n2-c1ccccc1] | 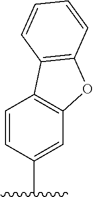 [SMILES: *c1ccc2c(c1)oc1ccccc12] |
| J17 | 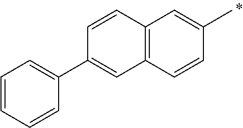 [SMILES: Cc1ccc2cc(-c3ccccc3)ccc2c1] | 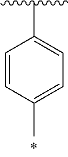 [SMILES: *c1ccc(C)cc1] | 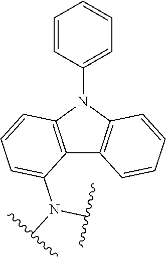 [SMILES: *N(*)c1cccc2c1c1ccccc1n2-c1ccccc1] | 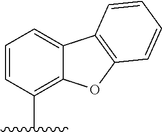 [SMILES: *c1cccc2c1oc1ccccc12] |
| J18 | 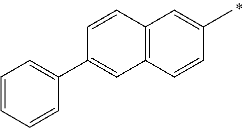 [SMILES: Cc1ccc2cc(-c3ccccc3)ccc2c1] | 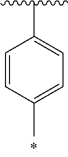 [SMILES: *c1ccc(C)cc1] | 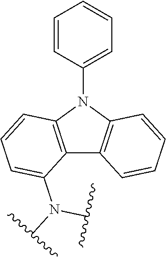 [SMILES: *N(*)c1cccc2c1c1ccccc1n2-c1ccccc1] | 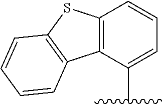 [SMILES: *c1cccc2sc3ccccc3c12] |
| J19 | 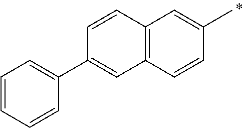 [SMILES: Cc1ccc2cc(-c3ccccc3)ccc2c1] | 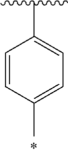 [SMILES: *c1ccc(C)cc1] | 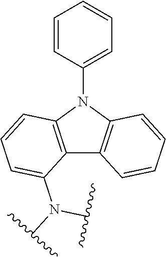 [SMILES: *N(*)c1cccc2c1c1ccccc1n2-c1ccccc1] | 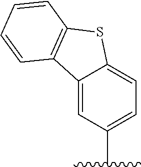 [SMILES: *c1ccc2sc3ccccc3c2c1] |
| J20 | 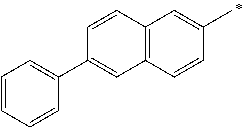 [SMILES: Cc1ccc2cc(-c3ccccc3)ccc2c1] | 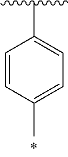 [SMILES: *c1ccc(C)cc1] | 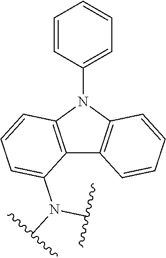 [SMILES: *N(*)c1cccc2c1c1ccccc1n2-c1ccccc1] | 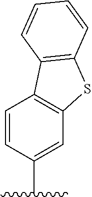 [SMILES: *c1ccc2c(c1)sc1ccccc12] |
| J21 | 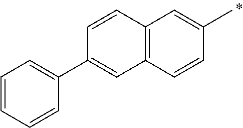 [SMILES: Cc1ccc2cc(-c3ccccc3)ccc2c1] | 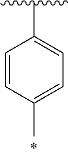 [SMILES: *c1ccc(C)cc1] | 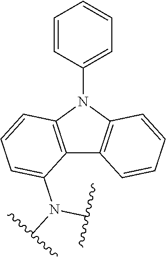 [SMILES: *N(*)c1cccc2c1c1ccccc1n2-c1ccccc1] | 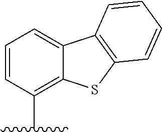 [SMILES: *c1cccc2c1sc1ccccc12] |
| J22 | 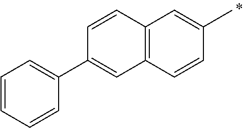 [SMILES: Cc1ccc2cc(-c3ccccc3)ccc2c1] | 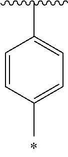 [SMILES: *c1ccc(C)cc1] | 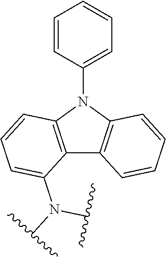 [SMILES: *N(*)c1cccc2c1c1ccccc1n2-c1ccccc1] | 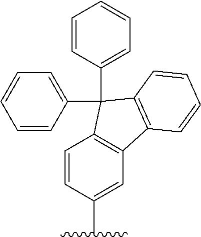 [SMILES: *c1ccc2c(c1)-c1ccccc1C2(c1ccccc1)c1ccccc1] |
| J23 | 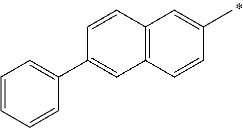 [SMILES: Cc1ccc2cc(-c3ccccc3)ccc2c1] | 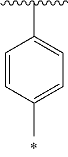 [SMILES: *c1ccc(C)cc1] | 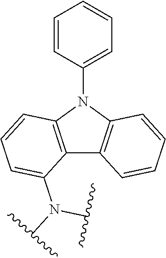 [SMILES: *N(*)c1cccc2c1c1ccccc1n2-c1ccccc1] | 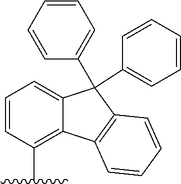 [SMILES: *c1cccc2c1-c1ccccc1C2(c1ccccc1)c1ccccc1] |
| J24 | 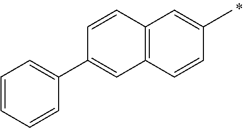 [SMILES: Cc1ccc2cc(-c3ccccc3)ccc2c1] | 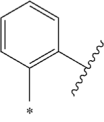 [SMILES: *c1ccccc1C] | 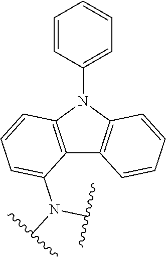 [SMILES: *N(*)c1cccc2c1c1ccccc1n2-c1ccccc1] | 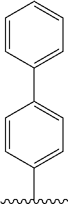 [SMILES: *c1ccc(-c2ccccc2)cc1] |
| J25 | 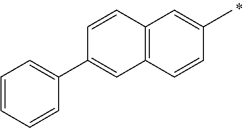 [SMILES: Cc1ccc2cc(-c3ccccc3)ccc2c1] | 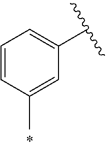 [SMILES: *c1cccc(C)c1] | 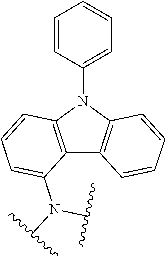 [SMILES: *N(*)c1cccc2c1c1ccccc1n2-c1ccccc1] | 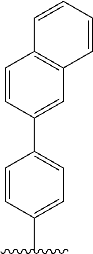 [SMILES: *c1ccc(-c2ccc3ccccc3c2)cc1] |
| J26 | 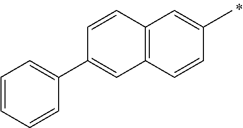 [SMILES: Cc1ccc2cc(-c3ccccc3)ccc2c1] | 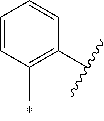 [SMILES: *c1ccccc1C] | 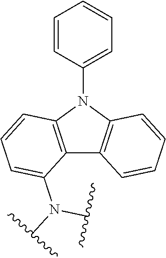 [SMILES: *N(*)c1cccc2c1c1ccccc1n2-c1ccccc1] | 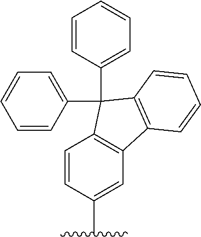 [SMILES: *c1ccc2c(c1)-c1ccccc1C2(c1ccccc1)c1ccccc1] |
| J27 | 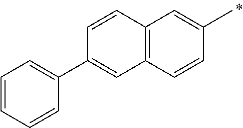 [SMILES: Cc1ccc2cc(-c3ccccc3)ccc2c1] | 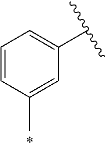 [SMILES: *c1cccc(C)c1] | 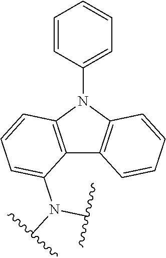 [SMILES: *N(*)c1cccc2c1c1ccccc1n2-c1ccccc1] | 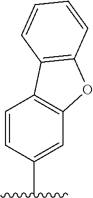 [SMILES: *c1ccc2c(c1)oc1ccccc12] |
| J28 | 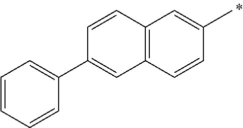 [SMILES: Cc1ccc2cc(-c3ccccc3)ccc2c1] | 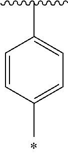 [SMILES: *c1ccc(C)cc1] | 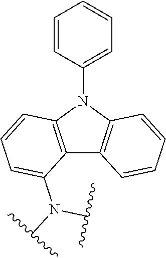 [SMILES: *N(*)c1cccc2c1c1ccccc1n2-c1ccccc1] | 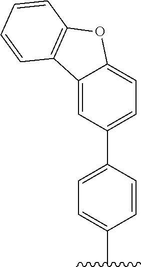 [SMILES: *c1ccc(-c2ccc3oc4ccccc4c3c2)cc1] |
| J29 | 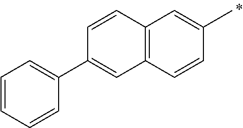 [SMILES: Cc1ccc2cc(-c3ccccc3)ccc2c1] | 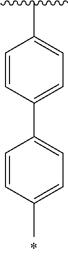 [SMILES: *c1ccc(-c2ccc(C)cc2)cc1] | 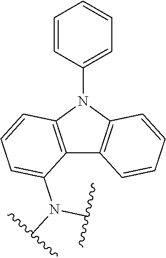 [SMILES: *N(*)c1cccc2c1c1ccccc1n2-c1ccccc1] | 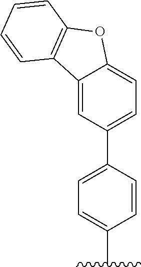 [SMILES: *c1ccc(-c2ccc3oc4ccccc4c3c2)cc1] |
| J30 | 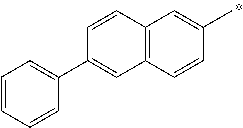 [SMILES: Cc1ccc2cc(-c3ccccc3)ccc2c1] | 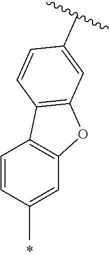 [SMILES: *c1ccc2c(c1)oc1cc(C)ccc12] | 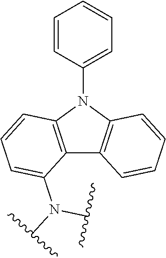 [SMILES: *N(*)c1cccc2c1c1ccccc1n2-c1ccccc1] | 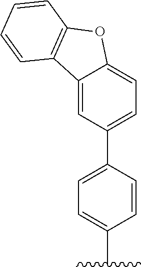 [SMILES: *c1ccc(-c2ccc3oc4ccccc4c3c2)cc1] |
| J31 | 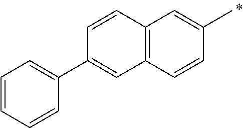 [SMILES: Cc1ccc2cc(-c3ccccc3)ccc2c1] | 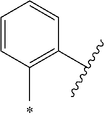 [SMILES: *c1ccccc1C] | 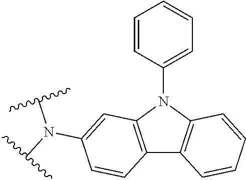 [SMILES: *N(*)c1ccc2c3ccccc3n(-c3ccccc3)c2c1] | 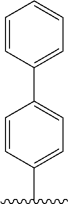 [SMILES: *c1ccc(-c2ccccc2)cc1] |
| J32 | 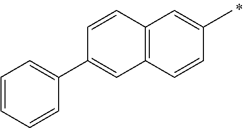 [SMILES: Cc1ccc2cc(-c3ccccc3)ccc2c1] | 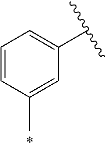 [SMILES: *c1cccc(C)c1] | 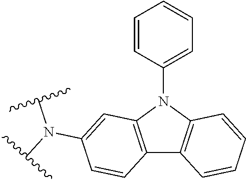 [SMILES: *N(*)c1ccc2c3ccccc3n(-c3ccccc3)c2c1] | 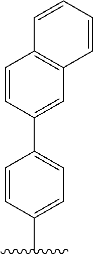 [SMILES: *c1ccc(-c2ccc3ccccc3c2)cc1] |
| J33 | 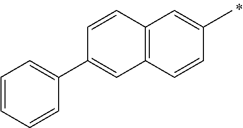 [SMILES: Cc1ccc2cc(-c3ccccc3)ccc2c1] | 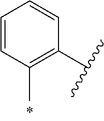 [SMILES: *c1ccccc1C] | 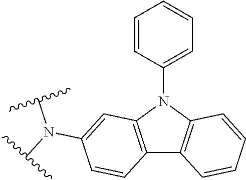 [SMILES: *N(*)c1ccc2c3ccccc3n(-c3ccccc3)c2c1] | 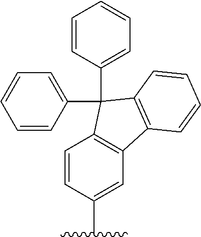 [SMILES: *c1ccc2c(c1)-c1ccccc1C2(c1ccccc1)c1ccccc1] |
| J34 | 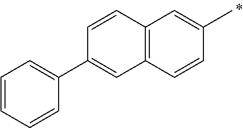 [SMILES: Cc1ccc2cc(-c3ccccc3)ccc2c1] | 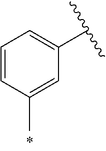 [SMILES: *c1cccc(C)c1] | 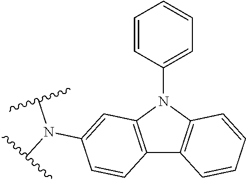 [SMILES: *N(*)c1ccc2c3ccccc3n(-c3ccccc3)c2c1] | 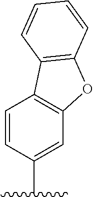 [SMILES: *c1ccc2c(c1)oc1ccccc12] |
| J35 | 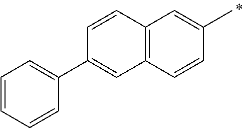 [SMILES: Cc1ccc2cc(-c3ccccc3)ccc2c1] | 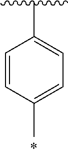 [SMILES: *c1ccc(C)cc1] | 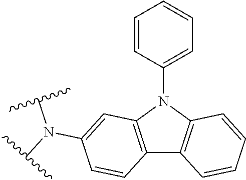 [SMILES: *N(*)c1ccc2c3ccccc3n(-c3ccccc3)c2c1] | 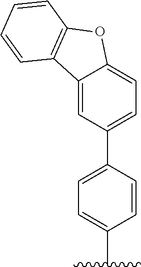 [SMILES: *c1ccc(-c2ccc3oc4ccccc4c3c2)cc1] |
| J36 | 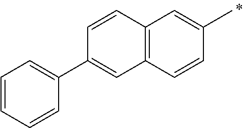 [SMILES: Cc1ccc2cc(-c3ccccc3)ccc2c1] | 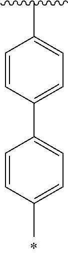 [SMILES: *c1ccc(-c2ccc(C)cc2)cc1] | 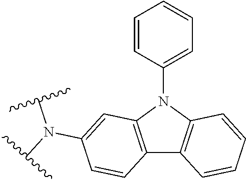 [SMILES: *N(*)c1ccc2c3ccccc3n(-c3ccccc3)c2c1] | 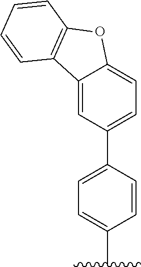 [SMILES: *c1ccc(-c2ccc3oc4ccccc4c3c2)cc1] |
| J37 | 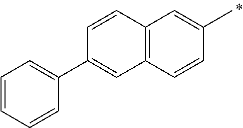 [SMILES: Cc1ccc2cc(-c3ccccc3)ccc2c1] | 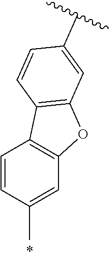 [SMILES: *c1ccc2c(c1)oc1cc(C)ccc12] | 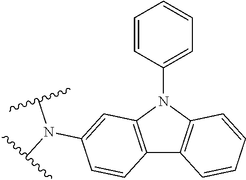 [SMILES: *N(*)c1ccc2c3ccccc3n(-c3ccccc3)c2c1] | 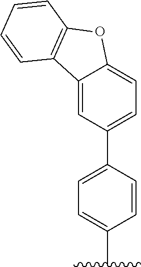 [SMILES: *c1ccc(-c2ccc3oc4ccccc4c3c2)cc1] |
| K1 | 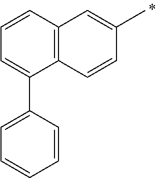 [SMILES: Cc1ccc2c(-c3ccccc3)cccc2c1] | 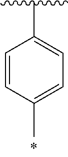 [SMILES: *c1ccc(C)cc1] | 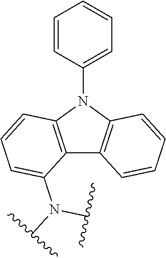 [SMILES: *N(*)c1cccc2c1c1ccccc1n2-c1ccccc1] | 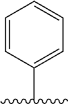 [SMILES: *c1ccccc1] |
| K2 | 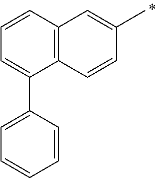 [SMILES: Cc1ccc2c(-c3ccccc3)cccc2c1] | 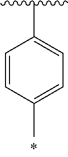 [SMILES: *c1ccc(C)cc1] | 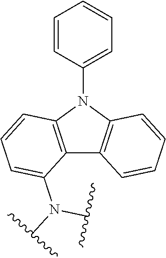 [SMILES: *N(*)c1cccc2c1c1ccccc1n2-c1ccccc1] | 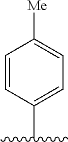 [SMILES: *c1ccc(C)cc1] |
| K3 | 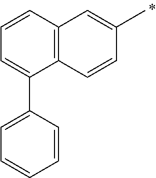 [SMILES: Cc1ccc2c(-c3ccccc3)cccc2c1] | 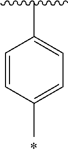 [SMILES: *c1ccc(C)cc1] | 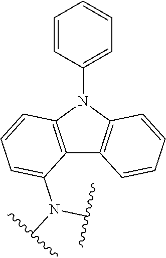 [SMILES: *N(*)c1cccc2c1c1ccccc1n2-c1ccccc1] | 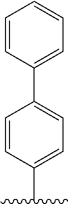 [SMILES: *c1ccc(-c2ccccc2)cc1] |
| K4 | 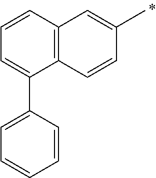 [SMILES: Cc1ccc2c(-c3ccccc3)cccc2c1] | 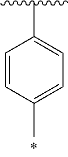 [SMILES: *c1ccc(C)cc1] | 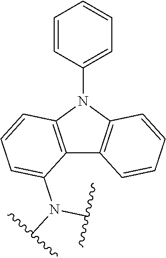 [SMILES: *N(*)c1cccc2c1c1ccccc1n2-c1ccccc1] | 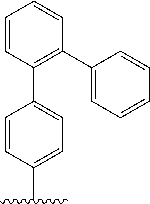 [SMILES: *c1ccc(-c2ccccc2-c2ccccc2)cc1] |
| K5 | 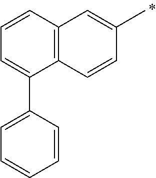 [SMILES: Cc1ccc2c(-c3ccccc3)cccc2c1] | 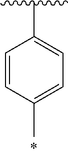 [SMILES: *c1ccc(C)cc1] | 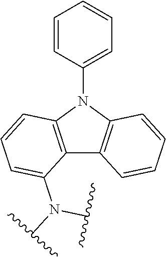 [SMILES: *N(*)c1cccc2c1c1ccccc1n2-c1ccccc1] | 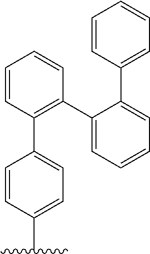 [SMILES: *c1ccc(-c2ccccc2-c2ccccc2-c2ccccc2)cc1] |
| K6 | 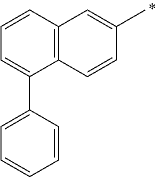 [SMILES: Cc1ccc2c(-c3ccccc3)cccc2c1] | 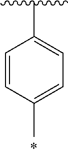 [SMILES: *c1ccc(C)cc1] | 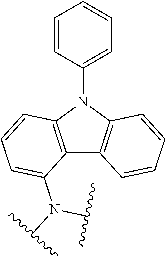 [SMILES: *N(*)c1cccc2c1c1ccccc1n2-c1ccccc1] | 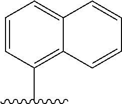 [SMILES: *c1cccc2ccccc12] |
| K7 | 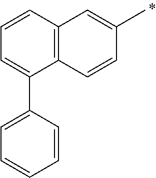 [SMILES: Cc1ccc2c(-c3ccccc3)cccc2c1] | 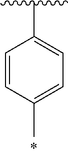 [SMILES: *c1ccc(C)cc1] | 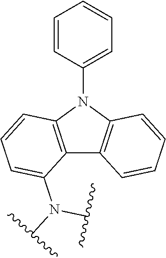 [SMILES: *N(*)c1cccc2c1c1ccccc1n2-c1ccccc1] | 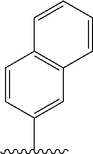 [SMILES: *c1ccc2ccccc2c1] |
| K8 | 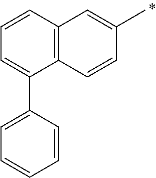 [SMILES: Cc1ccc2c(-c3ccccc3)cccc2c1] | 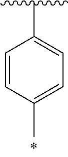 [SMILES: *c1ccc(C)cc1] | 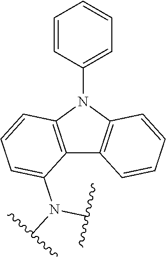 [SMILES: *N(*)c1cccc2c1c1ccccc1n2-c1ccccc1] | 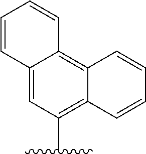 [SMILES: *c1cc2ccccc2c2ccccc12] |
| K9 | 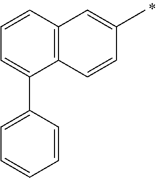 [SMILES: Cc1ccc2c(-c3ccccc3)cccc2c1] | 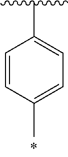 [SMILES: *c1ccc(C)cc1] | 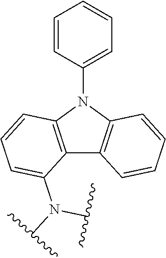 [SMILES: *N(*)c1cccc2c1c1ccccc1n2-c1ccccc1] | 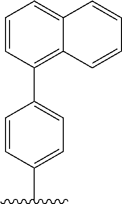 [SMILES: *c1ccc(-c2cccc3ccccc23)cc1] |
| K10 | 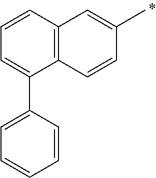 [SMILES: Cc1ccc2c(-c3ccccc3)cccc2c1] | 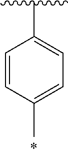 [SMILES: *c1ccc(C)cc1] | 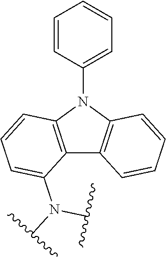 [SMILES: *N(*)c1cccc2c1c1ccccc1n2-c1ccccc1] | 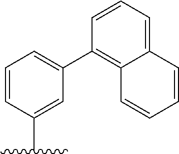 [SMILES: *c1cccc(-c2cccc3ccccc23)c1] |
| K11 | 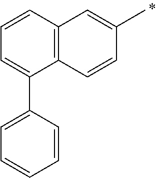 [SMILES: Cc1ccc2c(-c3ccccc3)cccc2c1] | 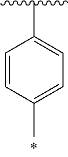 [SMILES: *c1ccc(C)cc1] | 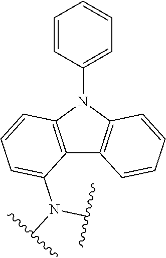 [SMILES: *N(*)c1cccc2c1c1ccccc1n2-c1ccccc1] | 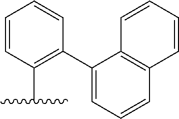 [SMILES: *c1ccccc1-c1cccc2ccccc12] |
| K12 | 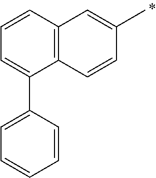 [SMILES: Cc1ccc2c(-c3ccccc3)cccc2c1] | 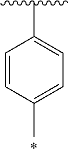 [SMILES: *c1ccc(C)cc1] | 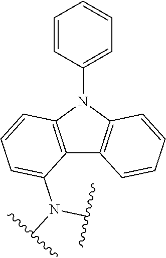 [SMILES: *N(*)c1cccc2c1c1ccccc1n2-c1ccccc1] | 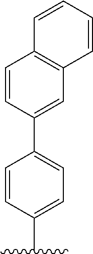 [SMILES: *c1ccc(-c2ccc3ccccc3c2)cc1] |
| K13 | 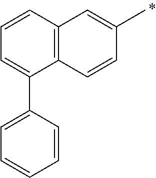 [SMILES: Cc1ccc2c(-c3ccccc3)cccc2c1] | 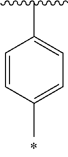 [SMILES: *c1ccc(C)cc1] | 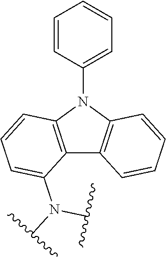 [SMILES: *N(*)c1cccc2c1c1ccccc1n2-c1ccccc1] | 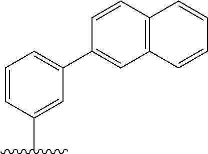 [SMILES: *c1cccc(-c2ccc3ccccc3c2)c1] |
| K14 | 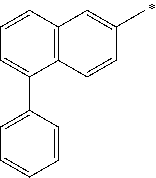 [SMILES: Cc1ccc2c(-c3ccccc3)cccc2c1] | 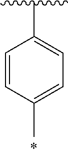 [SMILES: *c1ccc(C)cc1] | 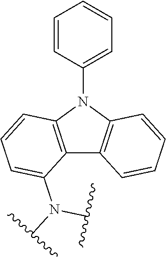 [SMILES: *N(*)c1cccc2c1c1ccccc1n2-c1ccccc1] | 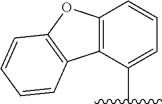 [SMILES: *c1cccc2oc3ccccc3c12] |
| K15 | 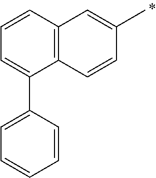 [SMILES: Cc1ccc2c(-c3ccccc3)cccc2c1] | 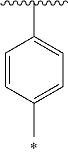 [SMILES: *c1ccc(C)cc1] | 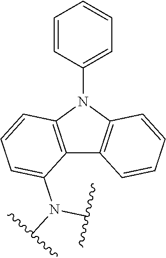 [SMILES: *N(*)c1cccc2c1c1ccccc1n2-c1ccccc1] | 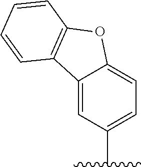 [SMILES: *c1ccc2oc3ccccc3c2c1] |
| K16 | 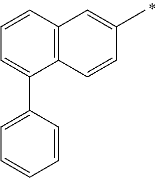 [SMILES: Cc1ccc2c(-c3ccccc3)cccc2c1] | 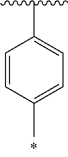 [SMILES: *c1ccc(C)cc1] | 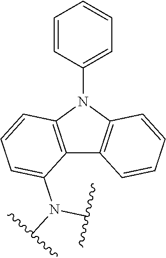 [SMILES: *N(*)c1cccc2c1c1ccccc1n2-c1ccccc1] | 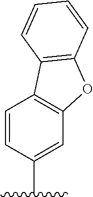 [SMILES: *c1ccc2c(c1)oc1ccccc12] |
| K17 | 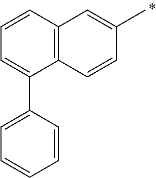 [SMILES: Cc1ccc2c(-c3ccccc3)cccc2c1] | 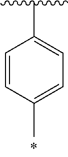 [SMILES: *c1ccc(C)cc1] | 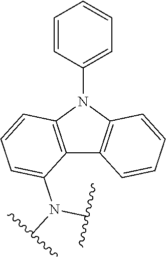 [SMILES: *N(*)c1cccc2c1c1ccccc1n2-c1ccccc1] | 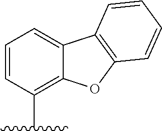 [SMILES: *c1cccc2c1oc1ccccc12] |
| K18 | 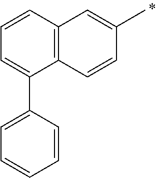 [SMILES: Cc1ccc2c(-c3ccccc3)cccc2c1] | 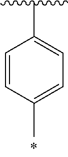 [SMILES: *c1ccc(C)cc1] | 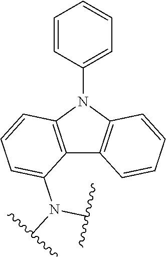 [SMILES: *N(*)c1cccc2c1c1ccccc1n2-c1ccccc1] | 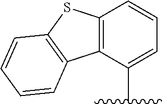 [SMILES: *c1cccc2sc3ccccc3c12] |
| K19 | 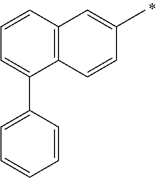 [SMILES: Cc1ccc2c(-c3ccccc3)cccc2c1] | 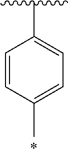 [SMILES: *c1ccc(C)cc1] | 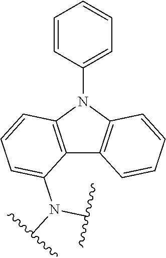 [SMILES: *N(*)c1cccc2c1c1ccccc1n2-c1ccccc1] | 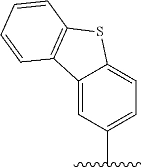 [SMILES: *c1ccc2sc3ccccc3c2c1] |
| K20 | 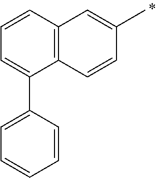 [SMILES: Cc1ccc2c(-c3ccccc3)cccc2c1] | 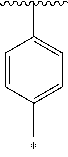 [SMILES: *c1ccc(C)cc1] | 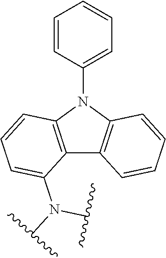 [SMILES: *N(*)c1cccc2c1c1ccccc1n2-c1ccccc1] | 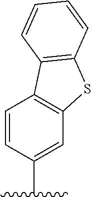 [SMILES: *c1ccc2c(c1)sc1ccccc12] |
| K21 | 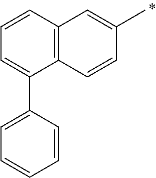 [SMILES: Cc1ccc2c(-c3ccccc3)cccc2c1] | 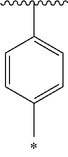 [SMILES: *c1ccc(C)cc1] | 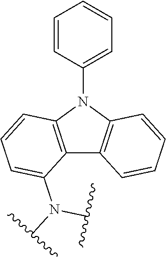 [SMILES: *N(*)c1cccc2c1c1ccccc1n2-c1ccccc1] | 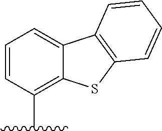 [SMILES: *c1cccc2c1sc1ccccc12] |
| K22 | 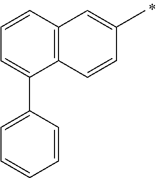 [SMILES: Cc1ccc2c(-c3ccccc3)cccc2c1] | 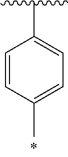 [SMILES: *c1ccc(C)cc1] | 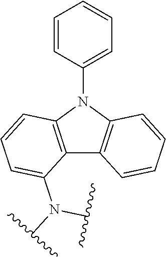 [SMILES: *N(*)c1cccc2c1c1ccccc1n2-c1ccccc1] | 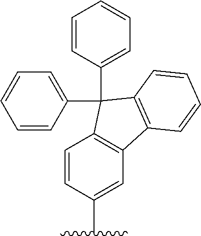 [SMILES: *c1ccc2c(c1)-c1ccccc1C2(c1ccccc1)c1ccccc1] |
| K23 | 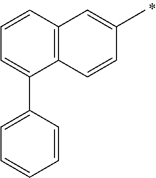 [SMILES: Cc1ccc2c(-c3ccccc3)cccc2c1] | 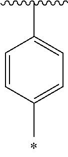 [SMILES: *c1ccc(C)cc1] | 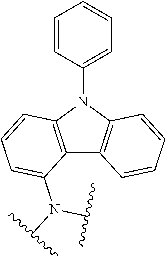 [SMILES: *N(*)c1cccc2c1c1ccccc1n2-c1ccccc1] | 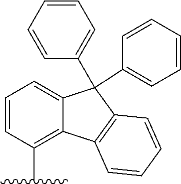 [SMILES: *c1cccc2c1-c1ccccc1C2(c1ccccc1)c1ccccc1] |
| K24 | 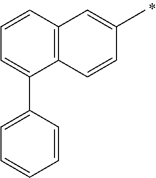 [SMILES: Cc1ccc2c(-c3ccccc3)cccc2c1] | 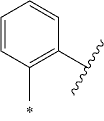 [SMILES: *c1ccccc1C] | 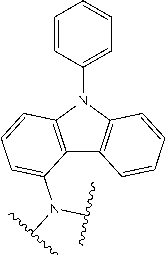 [SMILES: *N(*)c1cccc2c1c1ccccc1n2-c1ccccc1] | 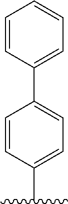 [SMILES: *c1ccc(-c2ccccc2)cc1] |
| K25 | 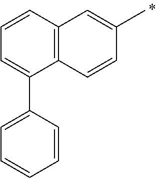 [SMILES: Cc1ccc2c(-c3ccccc3)cccc2c1] | 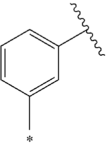 [SMILES: *c1cccc(C)c1] | 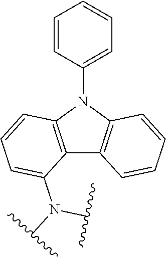 [SMILES: *N(*)c1cccc2c1c1ccccc1n2-c1ccccc1] | 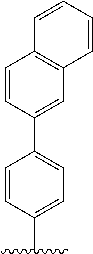 [SMILES: *c1ccc(-c2ccc3ccccc3c2)cc1] |
| K26 | 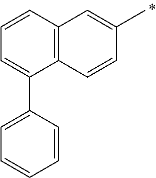 [SMILES: Cc1ccc2c(-c3ccccc3)cccc2c1] | 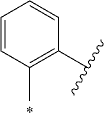 [SMILES: *c1ccccc1C] | 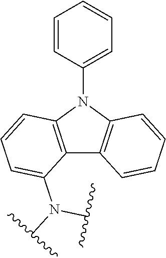 [SMILES: *N(*)c1cccc2c1c1ccccc1n2-c1ccccc1] | 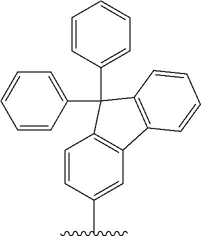 [SMILES: *c1ccc2c(c1)-c1ccccc1C2(c1ccccc1)c1ccccc1] |
| K27 | 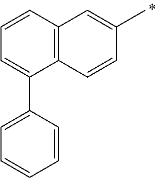 [SMILES: Cc1ccc2c(-c3ccccc3)cccc2c1] | 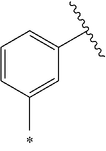 [SMILES: *c1cccc(C)c1] | 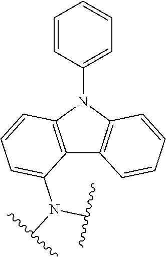 [SMILES: *N(*)c1cccc2c1c1ccccc1n2-c1ccccc1] | 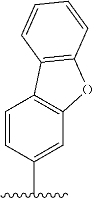 [SMILES: *c1ccc2c(c1)oc1ccccc12] |
| K28 | 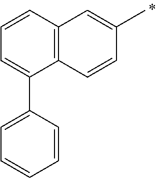 [SMILES: Cc1ccc2c(-c3ccccc3)cccc2c1] | 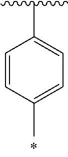 [SMILES: *c1ccc(C)cc1] | 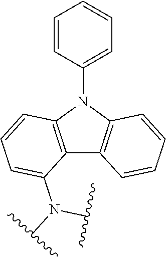 [SMILES: *N(*)c1cccc2c1c1ccccc1n2-c1ccccc1] | 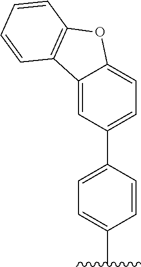 [SMILES: *c1ccc(-c2ccc3oc4ccccc4c3c2)cc1] |
| K29 | 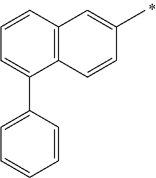 [SMILES: Cc1ccc2c(-c3ccccc3)cccc2c1] | 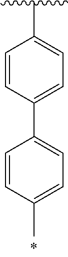 [SMILES: *c1ccc(-c2ccc(C)cc2)cc1] | 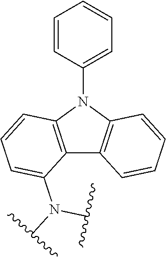 [SMILES: *N(*)c1cccc2c1c1ccccc1n2-c1ccccc1] | 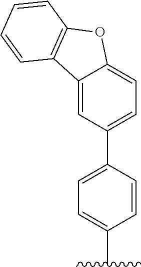 [SMILES: *c1ccc(-c2ccc3oc4ccccc4c3c2)cc1] |
| K30 | 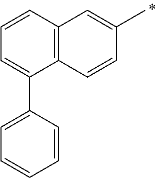 [SMILES: Cc1ccc2c(-c3ccccc3)cccc2c1] | 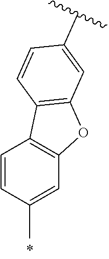 [SMILES: *c1ccc2c(c1)oc1cc(C)ccc12] | 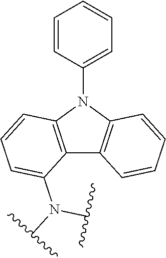 [SMILES: *N(*)c1cccc2c1c1ccccc1n2-c1ccccc1] | 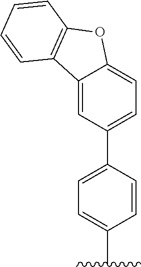 [SMILES: *c1ccc(-c2ccc3oc4ccccc4c3c2)cc1] |
| K31 | 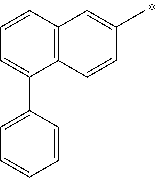 [SMILES: Cc1ccc2c(-c3ccccc3)cccc2c1] | 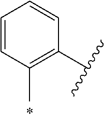 [SMILES: *c1ccccc1C] | 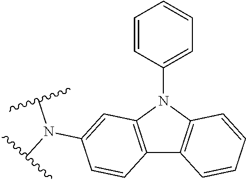 [SMILES: *N(*)c1ccc2c3ccccc3n(-c3ccccc3)c2c1] | 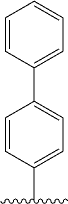 [SMILES: *c1ccc(-c2ccccc2)cc1] |
| K32 | 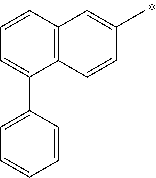 [SMILES: Cc1ccc2c(-c3ccccc3)cccc2c1] | 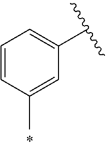 [SMILES: *c1cccc(C)c1] | 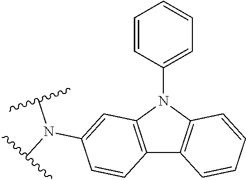 [SMILES: *N(*)c1ccc2c3ccccc3n(-c3ccccc3)c2c1] | 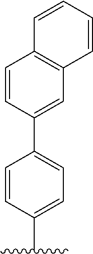 [SMILES: *c1ccc(-c2ccc3ccccc3c2)cc1] |
| K33 | 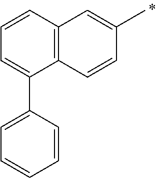 [SMILES: Cc1ccc2c(-c3ccccc3)cccc2c1] | 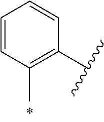 [SMILES: *c1ccccc1C] | 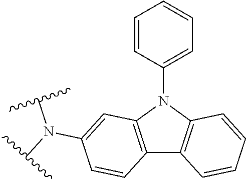 [SMILES: *N(*)c1ccc2c3ccccc3n(-c3ccccc3)c2c1] | 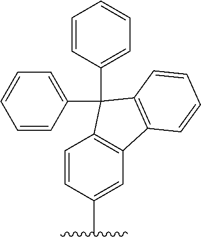 [SMILES: *c1ccc2c(c1)-c1ccccc1C2(c1ccccc1)c1ccccc1] |
| K34 | 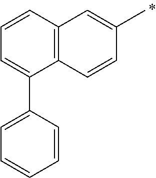 [SMILES: Cc1ccc2c(-c3ccccc3)cccc2c1] | 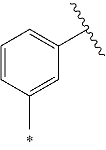 [SMILES: *c1cccc(C)c1] | 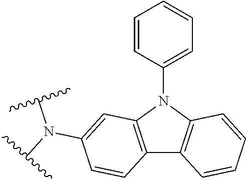 [SMILES: *N(*)c1ccc2c3ccccc3n(-c3ccccc3)c2c1] | 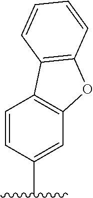 [SMILES: *c1ccc2c(c1)oc1ccccc12] |
| K35 | 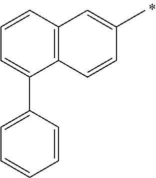 [SMILES: Cc1ccc2c(-c3ccccc3)cccc2c1] | 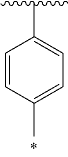 [SMILES: *c1ccc(C)cc1] | 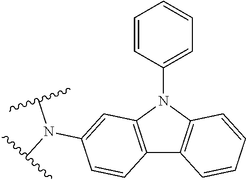 [SMILES: *N(*)c1ccc2c3ccccc3n(-c3ccccc3)c2c1] | 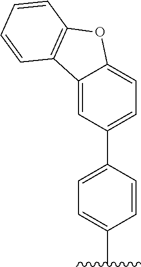 [SMILES: *c1ccc(-c2ccc3oc4ccccc4c3c2)cc1] |
| K36 | 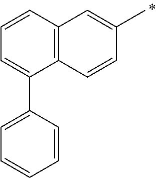 [SMILES: Cc1ccc2c(-c3ccccc3)cccc2c1] | 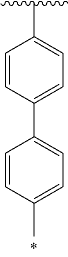 [SMILES: *c1ccc(-c2ccc(C)cc2)cc1] | 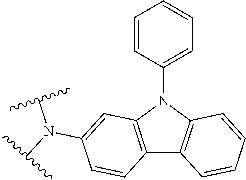 [SMILES: *N(*)c1ccc2c3ccccc3n(-c3ccccc3)c2c1] | 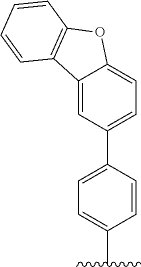 [SMILES: *c1ccc(-c2ccc3oc4ccccc4c3c2)cc1] |
| K37 | 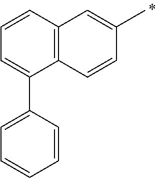 [SMILES: Cc1ccc2c(-c3ccccc3)cccc2c1] | 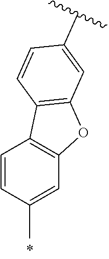 [SMILES: *c1ccc2c(c1)oc1cc(C)ccc12] | 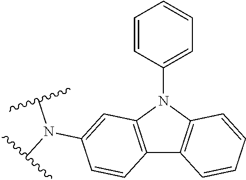 [SMILES: *N(*)c1ccc2c3ccccc3n(-c3ccccc3)c2c1] | 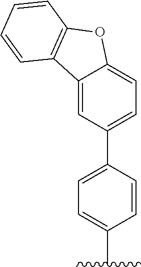 [SMILES: *c1ccc(-c2ccc3oc4ccccc4c3c2)cc1] |
| M1 | 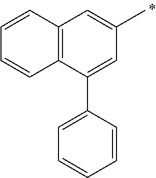 [SMILES: Cc1cc(-c2ccccc2)c2ccccc2c1] | 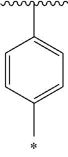 [SMILES: *c1ccc(C)cc1] | 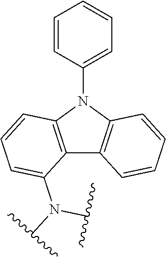 [SMILES: *N(*)c1cccc2c1c1ccccc1n2-c1ccccc1] | 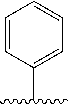 [SMILES: *c1ccccc1] |
| M2 | 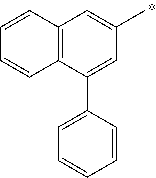 [SMILES: Cc1cc(-c2ccccc2)c2ccccc2c1] | 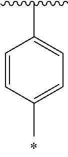 [SMILES: *c1ccc(C)cc1] | 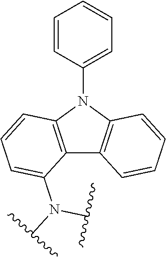 [SMILES: *N(*)c1cccc2c1c1ccccc1n2-c1ccccc1] | 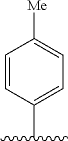 [SMILES: *c1ccc(C)cc1] |
| M3 | 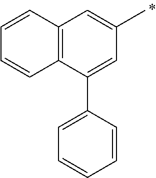 [SMILES: Cc1cc(-c2ccccc2)c2ccccc2c1] | 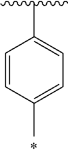 [SMILES: *c1ccc(C)cc1] | 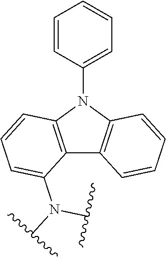 [SMILES: *N(*)c1cccc2c1c1ccccc1n2-c1ccccc1] | 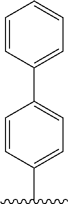 [SMILES: *c1ccc(-c2ccccc2)cc1] |
| M4 | 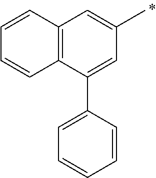 [SMILES: Cc1cc(-c2ccccc2)c2ccccc2c1] | 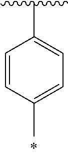 [SMILES: *c1ccc(C)cc1] | 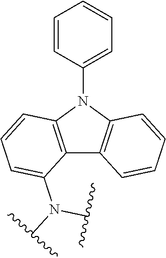 [SMILES: *N(*)c1cccc2c1c1ccccc1n2-c1ccccc1] | 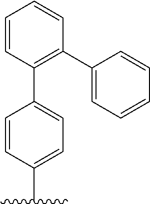 [SMILES: *c1ccc(-c2ccccc2-c2ccccc2)cc1] |
| M5 | 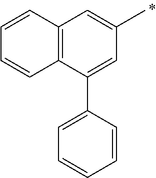 [SMILES: Cc1cc(-c2ccccc2)c2ccccc2c1] | 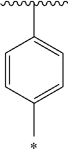 [SMILES: *c1ccc(C)cc1] | 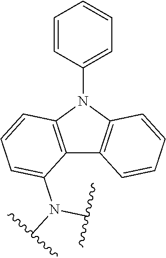 [SMILES: *N(*)c1cccc2c1c1ccccc1n2-c1ccccc1] | 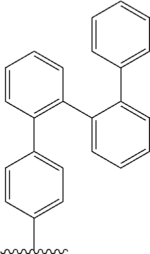 [SMILES: *c1ccc(-c2ccccc2-c2ccccc2-c2ccccc2)cc1] |
| M6 | 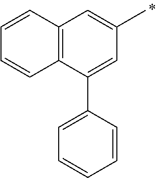 [SMILES: Cc1cc(-c2ccccc2)c2ccccc2c1] | 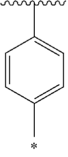 [SMILES: *c1ccc(C)cc1] | 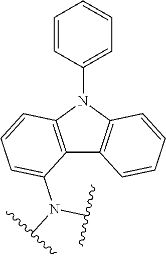 [SMILES: *N(*)c1cccc2c1c1ccccc1n2-c1ccccc1] | 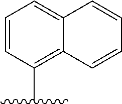 [SMILES: *c1cccc2ccccc12] |
| M7 | 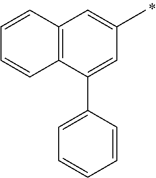 [SMILES: Cc1cc(-c2ccccc2)c2ccccc2c1] | 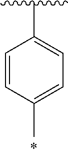 [SMILES: *c1ccc(C)cc1] | 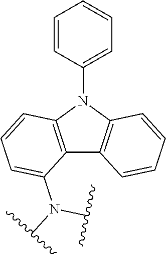 [SMILES: *N(*)c1cccc2c1c1ccccc1n2-c1ccccc1] | 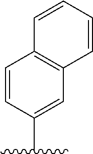 [SMILES: *c1ccc2ccccc2c1] |
| M8 | 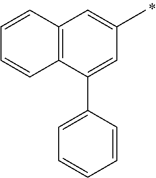 [SMILES: Cc1cc(-c2ccccc2)c2ccccc2c1] | 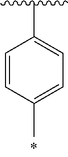 [SMILES: *c1ccc(C)cc1] | 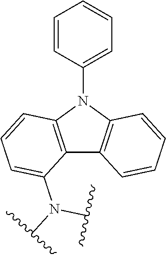 [SMILES: *N(*)c1cccc2c1c1ccccc1n2-c1ccccc1] | 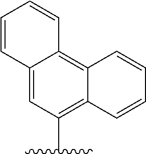 [SMILES: *c1cc2ccccc2c2ccccc12] |
| M9 | 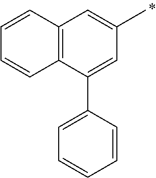 [SMILES: Cc1cc(-c2ccccc2)c2ccccc2c1] | 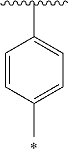 [SMILES: *c1ccc(C)cc1] | 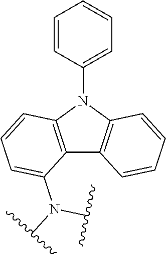 [SMILES: *N(*)c1cccc2c1c1ccccc1n2-c1ccccc1] | 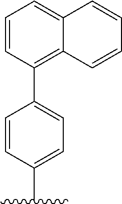 [SMILES: *c1ccc(-c2cccc3ccccc23)cc1] |
| M10 | 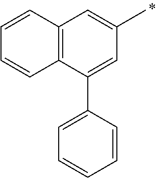 [SMILES: Cc1cc(-c2ccccc2)c2ccccc2c1] | 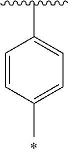 [SMILES: *c1ccc(C)cc1] | 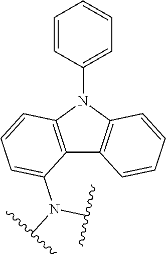 [SMILES: *N(*)c1cccc2c1c1ccccc1n2-c1ccccc1] | 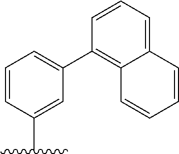 [SMILES: *c1cccc(-c2cccc3ccccc23)c1] |
| M11 | 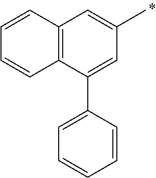 [SMILES: Cc1cc(-c2ccccc2)c2ccccc2c1] | 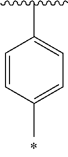 [SMILES: *c1ccc(C)cc1] | 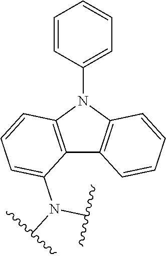 [SMILES: *N(*)c1cccc2c1c1ccccc1n2-c1ccccc1] | 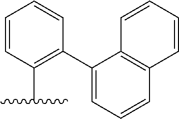 [SMILES: *c1ccccc1-c1cccc2ccccc12] |
| M12 | 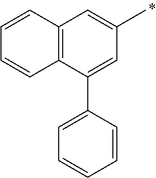 [SMILES: Cc1cc(-c2ccccc2)c2ccccc2c1] | 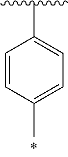 [SMILES: *c1ccc(C)cc1] | 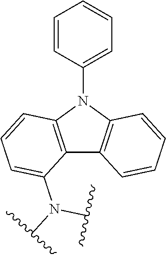 [SMILES: *N(*)c1cccc2c1c1ccccc1n2-c1ccccc1] | 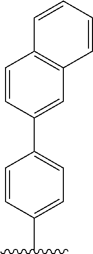 [SMILES: *c1ccc(-c2ccc3ccccc3c2)cc1] |
| M13 | 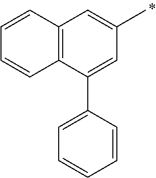 [SMILES: Cc1cc(-c2ccccc2)c2ccccc2c1] | 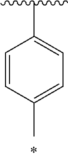 [SMILES: *c1ccc(C)cc1] | 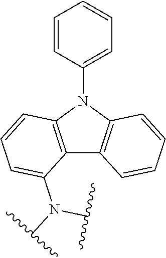 [SMILES: *N(*)c1cccc2c1c1ccccc1n2-c1ccccc1] | 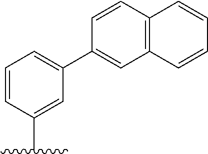 [SMILES: *c1cccc(-c2ccc3ccccc3c2)c1] |
| M14 | 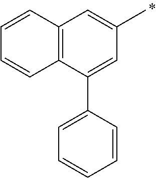 [SMILES: Cc1cc(-c2ccccc2)c2ccccc2c1] | 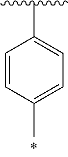 [SMILES: *c1ccc(C)cc1] | 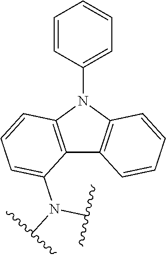 [SMILES: *N(*)c1cccc2c1c1ccccc1n2-c1ccccc1] | 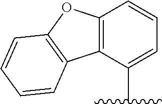 [SMILES: *c1cccc2oc3ccccc3c12] |
| M15 | 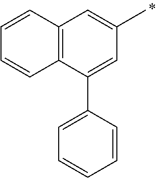 [SMILES: Cc1cc(-c2ccccc2)c2ccccc2c1] | 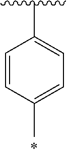 [SMILES: *c1ccc(C)cc1] | 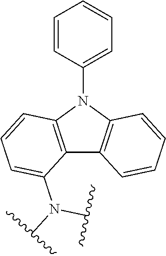 [SMILES: *N(*)c1cccc2c1c1ccccc1n2-c1ccccc1] | 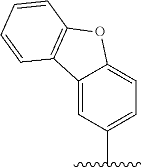 [SMILES: *c1ccc2oc3ccccc3c2c1] |
| M16 | 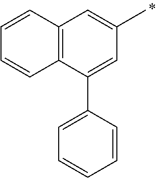 [SMILES: Cc1cc(-c2ccccc2)c2ccccc2c1] | 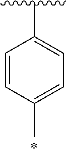 [SMILES: *c1ccc(C)cc1] | 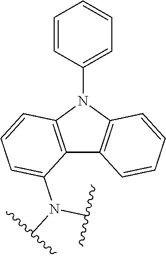 [SMILES: *N(*)c1cccc2c1c1ccccc1n2-c1ccccc1] | 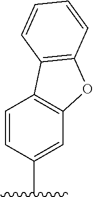 [SMILES: *c1ccc2c(c1)oc1ccccc12] |
| M17 | 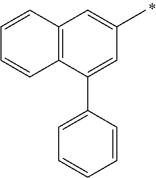 [SMILES: Cc1cc(-c2ccccc2)c2ccccc2c1] | 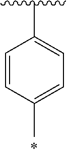 [SMILES: *c1ccc(C)cc1] | 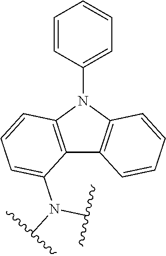 [SMILES: *N(*)c1cccc2c1c1ccccc1n2-c1ccccc1] | 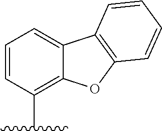 [SMILES: *c1cccc2c1oc1ccccc12] |
| M18 | 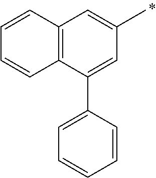 [SMILES: Cc1cc(-c2ccccc2)c2ccccc2c1] | 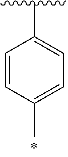 [SMILES: *c1ccc(C)cc1] | 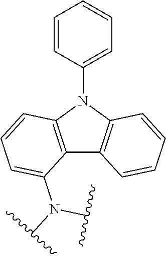 [SMILES: *N(*)c1cccc2c1c1ccccc1n2-c1ccccc1] | 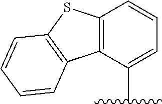 [SMILES: *c1cccc2sc3ccccc3c12] |
| M19 | 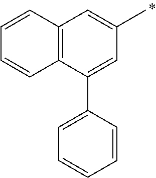 [SMILES: Cc1cc(-c2ccccc2)c2ccccc2c1] | 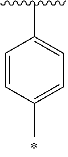 [SMILES: *c1ccc(C)cc1] | 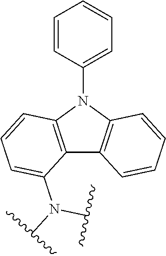 [SMILES: *N(*)c1cccc2c1c1ccccc1n2-c1ccccc1] | 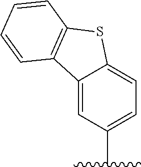 [SMILES: *c1ccc2sc3ccccc3c2c1] |
| M20 | 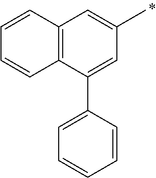 [SMILES: Cc1cc(-c2ccccc2)c2ccccc2c1] | 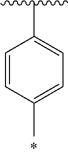 [SMILES: *c1ccc(C)cc1] | 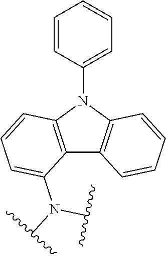 [SMILES: *N(*)c1cccc2c1c1ccccc1n2-c1ccccc1] | 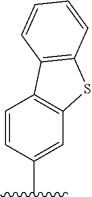 [SMILES: *c1ccc2c(c1)sc1ccccc12] |
| M21 | 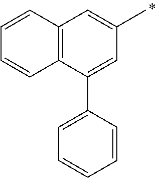 [SMILES: Cc1cc(-c2ccccc2)c2ccccc2c1] | 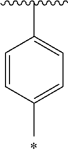 [SMILES: *c1ccc(C)cc1] | 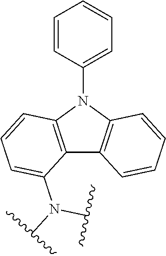 [SMILES: *N(*)c1cccc2c1c1ccccc1n2-c1ccccc1] | 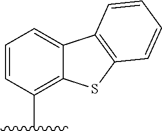 [SMILES: *c1cccc2c1sc1ccccc12] |
| M22 | 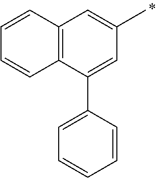 [SMILES: Cc1cc(-c2ccccc2)c2ccccc2c1] | 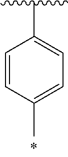 [SMILES: *c1ccc(C)cc1] | 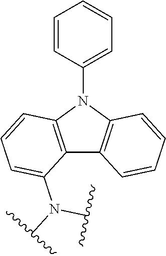 [SMILES: *N(*)c1cccc2c1c1ccccc1n2-c1ccccc1] | 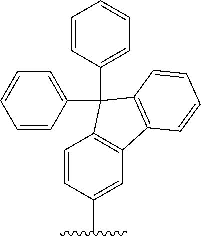 [SMILES: *c1ccc2c(c1)-c1ccccc1C2(c1ccccc1)c1ccccc1] |
| M23 | 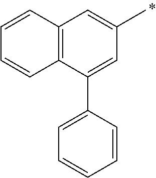 [SMILES: Cc1cc(-c2ccccc2)c2ccccc2c1] | 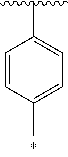 [SMILES: *c1ccc(C)cc1] | 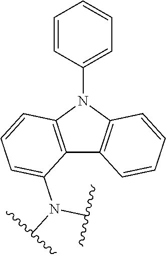 [SMILES: *N(*)c1cccc2c1c1ccccc1n2-c1ccccc1] | 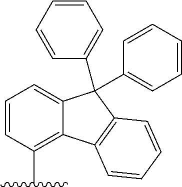 [SMILES: *c1cccc2c1-c1ccccc1C2(c1ccccc1)c1ccccc1] |
| M24 | 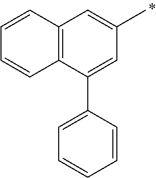 [SMILES: Cc1cc(-c2ccccc2)c2ccccc2c1] | 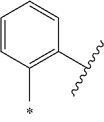 [SMILES: *c1ccccc1C] | 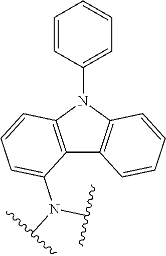 [SMILES: *N(*)c1cccc2c1c1ccccc1n2-c1ccccc1] | 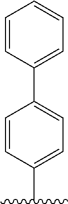 [SMILES: *c1ccc(-c2ccccc2)cc1] |
| M25 | 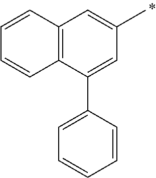 [SMILES: Cc1cc(-c2ccccc2)c2ccccc2c1] | 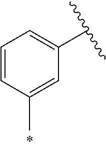 [SMILES: *c1cccc(C)c1] | 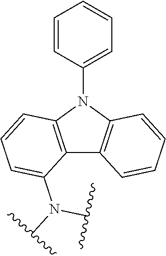 [SMILES: *N(*)c1cccc2c1c1ccccc1n2-c1ccccc1] | 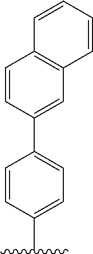 [SMILES: *c1ccc(-c2ccc3ccccc3c2)cc1] |
| M26 | 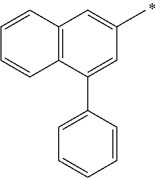 [SMILES: Cc1cc(-c2ccccc2)c2ccccc2c1] | 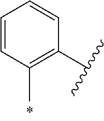 [SMILES: *c1ccccc1C] | 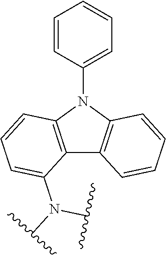 [SMILES: *N(*)c1cccc2c1c1ccccc1n2-c1ccccc1] | 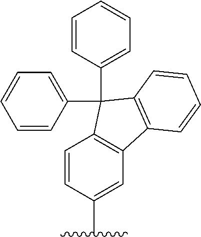 [SMILES: *c1ccc2c(c1)-c1ccccc1C2(c1ccccc1)c1ccccc1] |
| M27 | 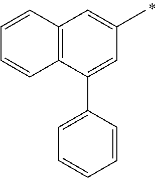 [SMILES: Cc1cc(-c2ccccc2)c2ccccc2c1] | 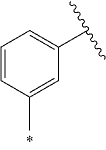 [SMILES: *c1cccc(C)c1] | 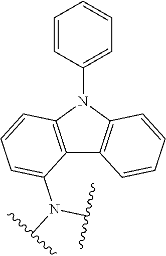 [SMILES: *N(*)c1cccc2c1c1ccccc1n2-c1ccccc1] | 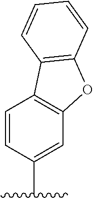 [SMILES: *c1ccc2c(c1)oc1ccccc12] |
| M28 | 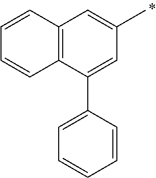 [SMILES: Cc1cc(-c2ccccc2)c2ccccc2c1] | 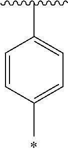 [SMILES: *c1ccc(C)cc1] | 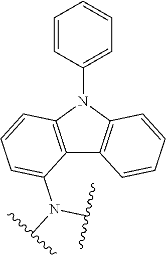 [SMILES: *N(*)c1cccc2c1c1ccccc1n2-c1ccccc1] | 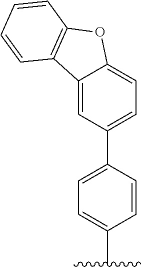 [SMILES: *c1ccc(-c2ccc3oc4ccccc4c3c2)cc1] |
| M29 | 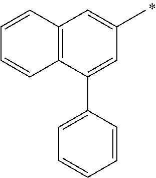 [SMILES: Cc1cc(-c2ccccc2)c2ccccc2c1] | 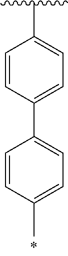 [SMILES: *c1ccc(-c2ccc(C)cc2)cc1] | 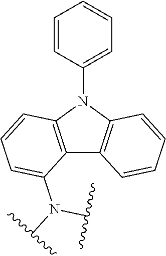 [SMILES: *N(*)c1cccc2c1c1ccccc1n2-c1ccccc1] | 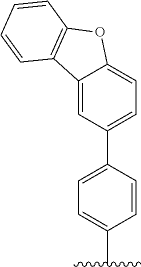 [SMILES: *c1ccc(-c2ccc3oc4ccccc4c3c2)cc1] |
| M30 | 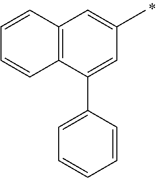 [SMILES: Cc1cc(-c2ccccc2)c2ccccc2c1] | 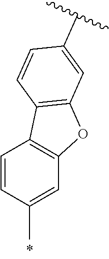 [SMILES: *c1ccc2c(c1)oc1cc(C)ccc12] | 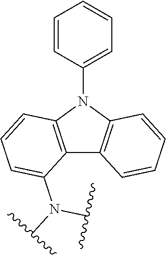 [SMILES: *N(*)c1cccc2c1c1ccccc1n2-c1ccccc1] | 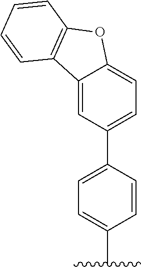 [SMILES: *c1ccc(-c2ccc3oc4ccccc4c3c2)cc1] |
| M31 | 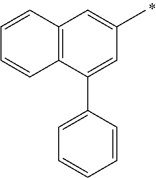 [SMILES: Cc1cc(-c2ccccc2)c2ccccc2c1] | 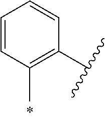 [SMILES: *c1ccccc1C] | 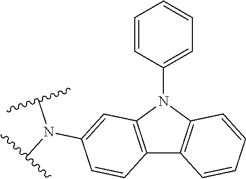 [SMILES: *N(*)c1ccc2c3ccccc3n(-c3ccccc3)c2c1] | 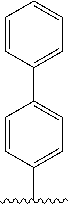 [SMILES: *c1ccc(-c2ccccc2)cc1] |
| M32 | 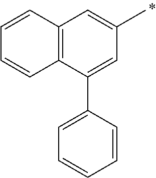 [SMILES: Cc1cc(-c2ccccc2)c2ccccc2c1] | 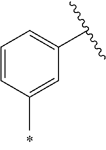 [SMILES: *c1cccc(C)c1] | 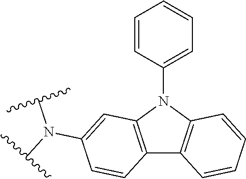 [SMILES: *N(*)c1ccc2c3ccccc3n(-c3ccccc3)c2c1] | 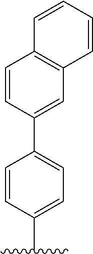 [SMILES: *c1ccc(-c2ccc3ccccc3c2)cc1] |
| M33 | 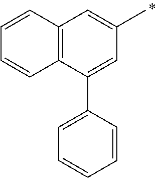 [SMILES: Cc1cc(-c2ccccc2)c2ccccc2c1] | 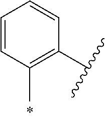 [SMILES: *c1ccccc1C] | 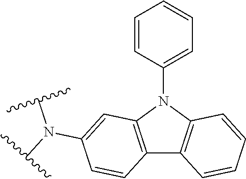 [SMILES: *N(*)c1ccc2c3ccccc3n(-c3ccccc3)c2c1] | 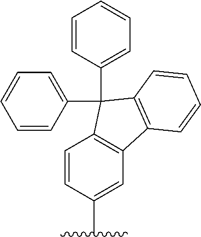 [SMILES: *c1ccc2c(c1)-c1ccccc1C2(c1ccccc1)c1ccccc1] |
| M34 | 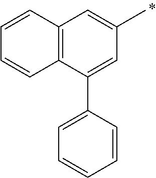 [SMILES: Cc1cc(-c2ccccc2)c2ccccc2c1] | 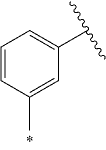 [SMILES: *c1cccc(C)c1] | 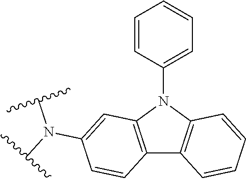 [SMILES: *N(*)c1ccc2c3ccccc3n(-c3ccccc3)c2c1] | 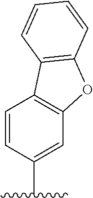 [SMILES: *c1ccc2c(c1)oc1ccccc12] |
| M35 | 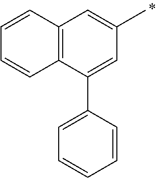 [SMILES: Cc1cc(-c2ccccc2)c2ccccc2c1] | 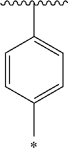 [SMILES: *c1ccc(C)cc1] | 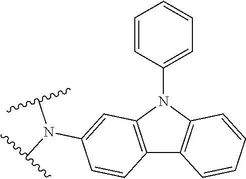 [SMILES: *N(*)c1ccc2c3ccccc3n(-c3ccccc3)c2c1] | 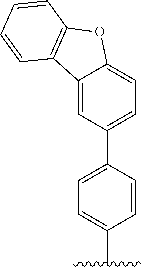 [SMILES: *c1ccc(-c2ccc3oc4ccccc4c3c2)cc1] |
| M36 | 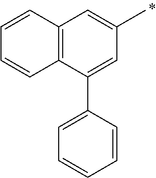 [SMILES: Cc1cc(-c2ccccc2)c2ccccc2c1] | 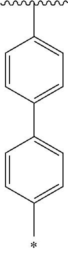 [SMILES: *c1ccc(-c2ccc(C)cc2)cc1] | 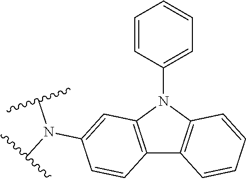 [SMILES: *N(*)c1ccc2c3ccccc3n(-c3ccccc3)c2c1] | 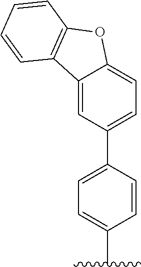 [SMILES: *c1ccc(-c2ccc3oc4ccccc4c3c2)cc1] |
| M37 | 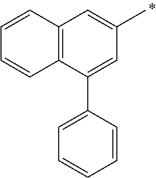 [SMILES: Cc1cc(-c2ccccc2)c2ccccc2c1] | 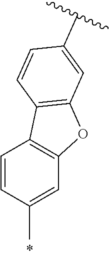 [SMILES: *c1ccc2c(c1)oc1cc(C)ccc12] | 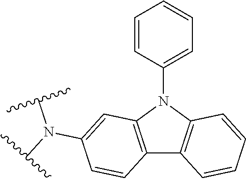 [SMILES: *N(*)c1ccc2c3ccccc3n(-c3ccccc3)c2c1] | 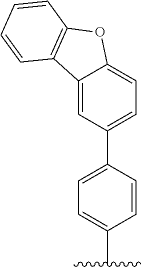 [SMILES: *c1ccc(-c2ccc3oc4ccccc4c3c2)cc1] |
| N1 | 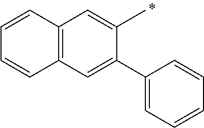 [SMILES: Cc1cc2ccccc2cc1-c1ccccc1] | 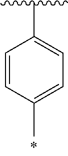 [SMILES: *c1ccc(C)cc1] | 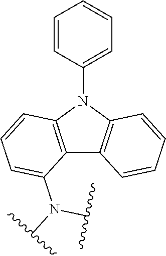 [SMILES: *N(*)c1cccc2c1c1ccccc1n2-c1ccccc1] | 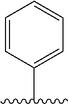 [SMILES: *c1ccccc1] |
| N2 | 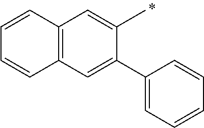 [SMILES: Cc1cc2ccccc2cc1-c1ccccc1] | 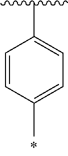 [SMILES: *c1ccc(C)cc1] | 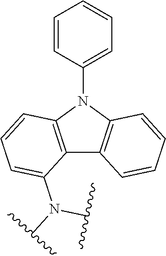 [SMILES: *N(*)c1cccc2c1c1ccccc1n2-c1ccccc1] | 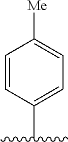 [SMILES: *c1ccc(C)cc1] |
| N3 | 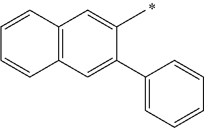 [SMILES: Cc1cc2ccccc2cc1-c1ccccc1] | 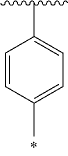 [SMILES: *c1ccc(C)cc1] | 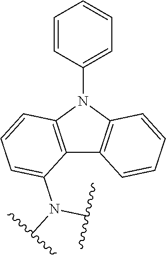 [SMILES: *N(*)c1cccc2c1c1ccccc1n2-c1ccccc1] | 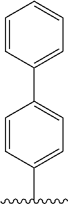 [SMILES: *c1ccc(-c2ccccc2)cc1] |
| N4 | 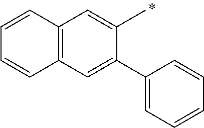 [SMILES: Cc1cc2ccccc2cc1-c1ccccc1] | 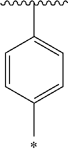 [SMILES: *c1ccc(C)cc1] | 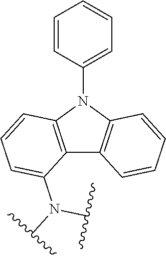 [SMILES: *N(*)c1cccc2c1c1ccccc1n2-c1ccccc1] | 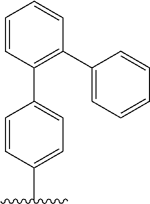 [SMILES: *c1ccc(-c2ccccc2-c2ccccc2)cc1] |
| N5 | 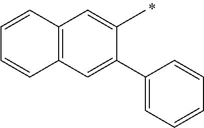 [SMILES: Cc1cc2ccccc2cc1-c1ccccc1] | 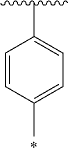 [SMILES: *c1ccc(C)cc1] | 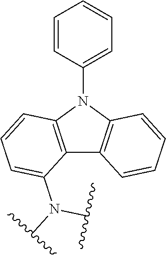 [SMILES: *N(*)c1cccc2c1c1ccccc1n2-c1ccccc1] | 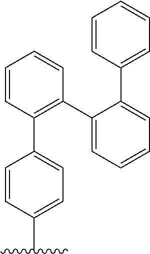 [SMILES: *c1ccc(-c2ccccc2-c2ccccc2-c2ccccc2)cc1] |
| N6 | 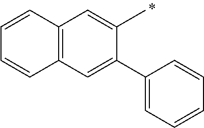 [SMILES: Cc1cc2ccccc2cc1-c1ccccc1] | 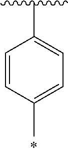 [SMILES: *c1ccc(C)cc1] | 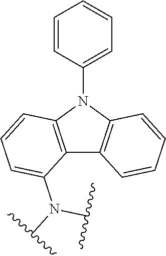 [SMILES: *N(*)c1cccc2c1c1ccccc1n2-c1ccccc1] | 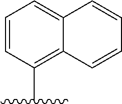 [SMILES: *c1cccc2ccccc12] |
| N7 | 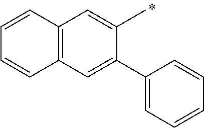 [SMILES: Cc1cc2ccccc2cc1-c1ccccc1] | 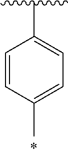 [SMILES: *c1ccc(C)cc1] | 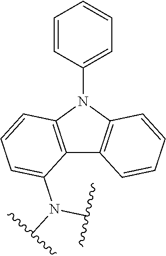 [SMILES: *N(*)c1cccc2c1c1ccccc1n2-c1ccccc1] | 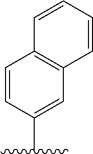 [SMILES: *c1ccc2ccccc2c1] |
| N8 | 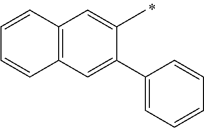 [SMILES: Cc1cc2ccccc2cc1-c1ccccc1] | 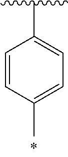 [SMILES: *c1ccc(C)cc1] | 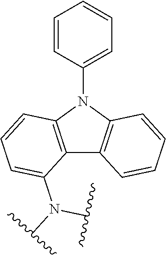 [SMILES: *N(*)c1cccc2c1c1ccccc1n2-c1ccccc1] | 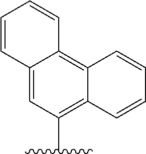 [SMILES: *c1cc2ccccc2c2ccccc12] |
| N9 | 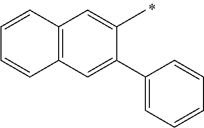 [SMILES: Cc1cc2ccccc2cc1-c1ccccc1] | 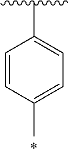 [SMILES: *c1ccc(C)cc1] | 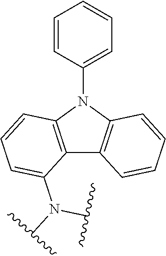 [SMILES: *N(*)c1cccc2c1c1ccccc1n2-c1ccccc1] | 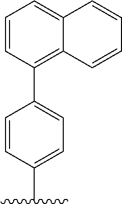 [SMILES: *c1ccc(-c2cccc3ccccc23)cc1] |
| N10 | 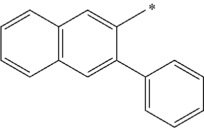 [SMILES: Cc1cc2ccccc2cc1-c1ccccc1] | 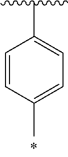 [SMILES: *c1ccc(C)cc1] | 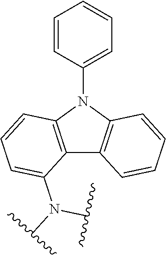 [SMILES: *N(*)c1cccc2c1c1ccccc1n2-c1ccccc1] | 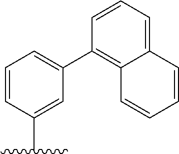 [SMILES: *c1cccc(-c2cccc3ccccc23)c1] |
| N11 | 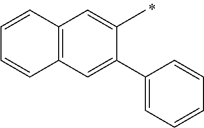 [SMILES: Cc1cc2ccccc2cc1-c1ccccc1] | 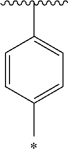 [SMILES: *c1ccc(C)cc1] | 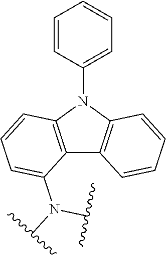 [SMILES: *N(*)c1cccc2c1c1ccccc1n2-c1ccccc1] | 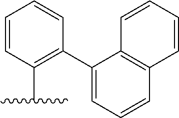 [SMILES: *c1ccccc1-c1cccc2ccccc12] |
| N12 | 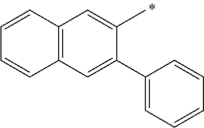 [SMILES: Cc1cc2ccccc2cc1-c1ccccc1] | 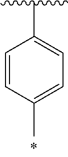 [SMILES: *c1ccc(C)cc1] | 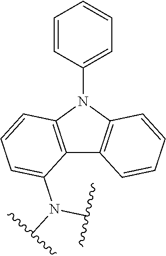 [SMILES: *N(*)c1cccc2c1c1ccccc1n2-c1ccccc1] | 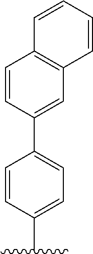 [SMILES: *c1ccc(-c2ccc3ccccc3c2)cc1] |
| N13 | 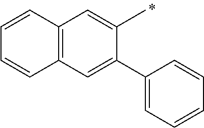 [SMILES: Cc1cc2ccccc2cc1-c1ccccc1] | 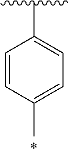 [SMILES: *c1ccc(C)cc1] | 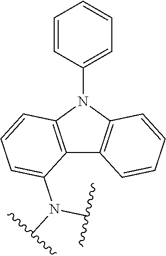 [SMILES: *N(*)c1cccc2c1c1ccccc1n2-c1ccccc1] | 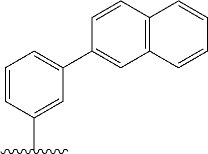 [SMILES: *c1cccc(-c2ccc3ccccc3c2)c1] |
| N14 | 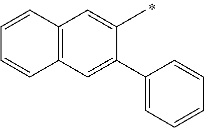 [SMILES: Cc1cc2ccccc2cc1-c1ccccc1] | 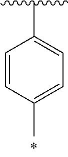 [SMILES: *c1ccc(C)cc1] | 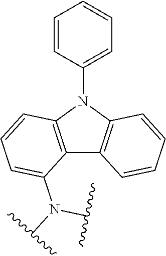 [SMILES: *N(*)c1cccc2c1c1ccccc1n2-c1ccccc1] | 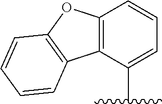 [SMILES: *c1cccc2oc3ccccc3c12] |
| N15 | 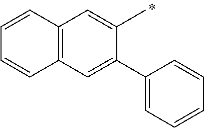 [SMILES: Cc1cc2ccccc2cc1-c1ccccc1] | 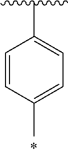 [SMILES: *c1ccc(C)cc1] | 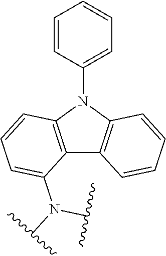 [SMILES: *N(*)c1cccc2c1c1ccccc1n2-c1ccccc1] | 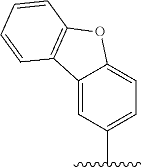 [SMILES: *c1ccc2oc3ccccc3c2c1] |
| N16 | 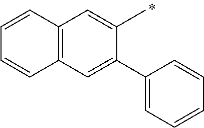 [SMILES: Cc1cc2ccccc2cc1-c1ccccc1] | 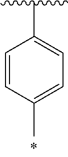 [SMILES: *c1ccc(C)cc1] | 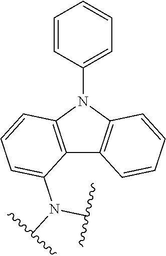 [SMILES: *N(*)c1cccc2c1c1ccccc1n2-c1ccccc1] | 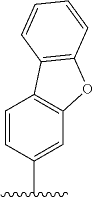 [SMILES: *c1ccc2c(c1)oc1ccccc12] |
| N17 | 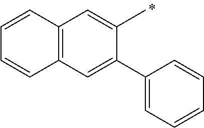 [SMILES: Cc1cc2ccccc2cc1-c1ccccc1] | 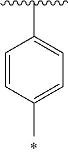 [SMILES: *c1ccc(C)cc1] | 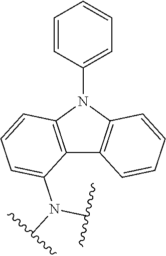 [SMILES: *N(*)c1cccc2c1c1ccccc1n2-c1ccccc1] | 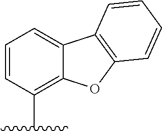 [SMILES: *c1cccc2c1oc1ccccc12] |
| N18 | 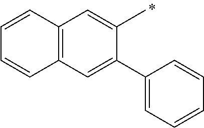 [SMILES: Cc1cc2ccccc2cc1-c1ccccc1] | 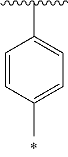 [SMILES: *c1ccc(C)cc1] | 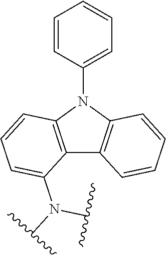 [SMILES: *N(*)c1cccc2c1c1ccccc1n2-c1ccccc1] | 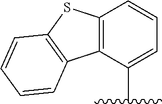 [SMILES: *c1cccc2sc3ccccc3c12] |
| N19 | 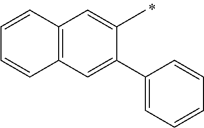 [SMILES: Cc1cc2ccccc2cc1-c1ccccc1] | 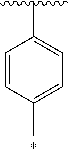 [SMILES: *c1ccc(C)cc1] | 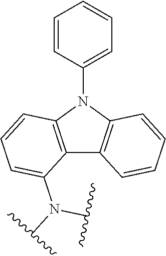 [SMILES: *N(*)c1cccc2c1c1ccccc1n2-c1ccccc1] | 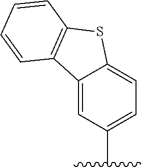 [SMILES: *c1ccc2sc3ccccc3c2c1] |
| N20 | 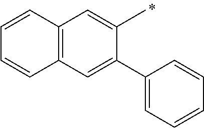 [SMILES: Cc1cc2ccccc2cc1-c1ccccc1] | 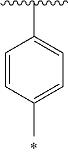 [SMILES: *c1ccc(C)cc1] | 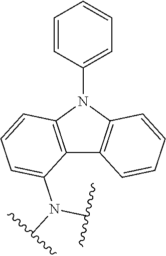 [SMILES: *N(*)c1cccc2c1c1ccccc1n2-c1ccccc1] | 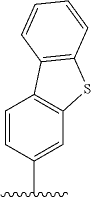 [SMILES: *c1ccc2c(c1)sc1ccccc12] |
| N21 | 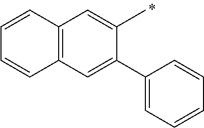 [SMILES: Cc1cc2ccccc2cc1-c1ccccc1] | 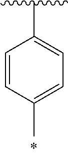 [SMILES: *c1ccc(C)cc1] | 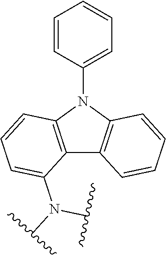 [SMILES: *N(*)c1cccc2c1c1ccccc1n2-c1ccccc1] | 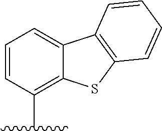 [SMILES: *c1cccc2c1sc1ccccc12] |
| N22 | 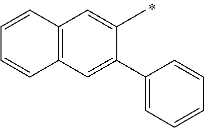 [SMILES: Cc1cc2ccccc2cc1-c1ccccc1] | 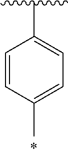 [SMILES: *c1ccc(C)cc1] | 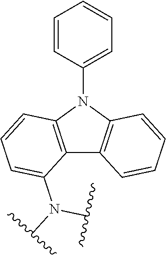 [SMILES: *N(*)c1cccc2c1c1ccccc1n2-c1ccccc1] | 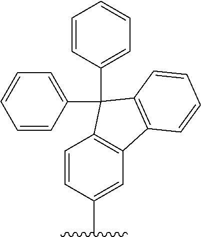 [SMILES: *c1ccc2c(c1)-c1ccccc1C2(c1ccccc1)c1ccccc1] |
| N23 | 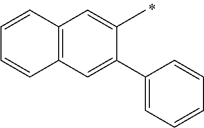 [SMILES: Cc1cc2ccccc2cc1-c1ccccc1] | 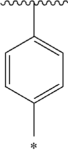 [SMILES: *c1ccc(C)cc1] | 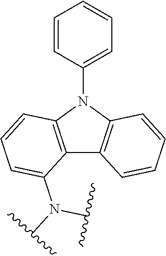 [SMILES: *N(*)c1cccc2c1c1ccccc1n2-c1ccccc1] | 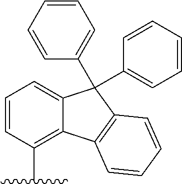 [SMILES: *c1cccc2c1-c1ccccc1C2(c1ccccc1)c1ccccc1] |
| N24 | 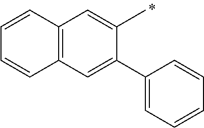 [SMILES: Cc1cc2ccccc2cc1-c1ccccc1] | 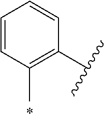 [SMILES: *c1ccccc1C] | 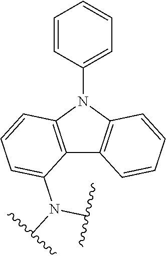 [SMILES: *N(*)c1cccc2c1c1ccccc1n2-c1ccccc1] | 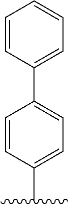 [SMILES: *c1ccc(-c2ccccc2)cc1] |
| N25 | 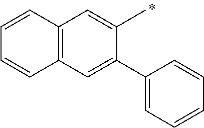 [SMILES: Cc1cc2ccccc2cc1-c1ccccc1] | 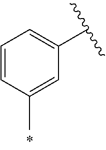 [SMILES: *c1cccc(C)c1] | 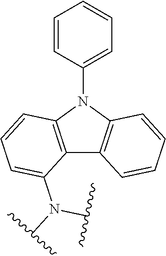 [SMILES: *N(*)c1cccc2c1c1ccccc1n2-c1ccccc1] | 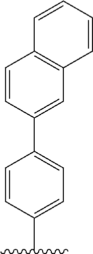 [SMILES: *c1ccc(-c2ccc3ccccc3c2)cc1] |
| N26 | 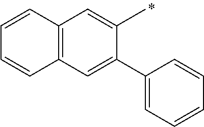 [SMILES: Cc1cc2ccccc2cc1-c1ccccc1] | 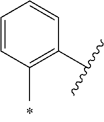 [SMILES: *c1ccccc1C] | 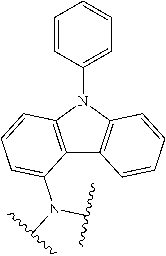 [SMILES: *N(*)c1cccc2c1c1ccccc1n2-c1ccccc1] | 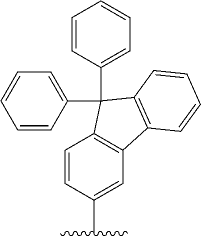 [SMILES: *c1ccc2c(c1)-c1ccccc1C2(c1ccccc1)c1ccccc1] |
| N27 | 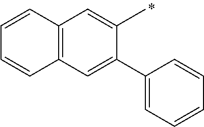 [SMILES: Cc1cc2ccccc2cc1-c1ccccc1] | 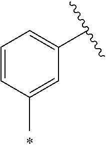 [SMILES: *c1cccc(C)c1] | 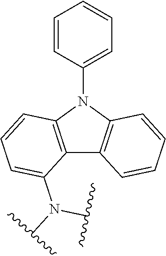 [SMILES: *N(*)c1cccc2c1c1ccccc1n2-c1ccccc1] | 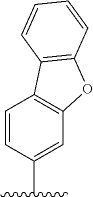 [SMILES: *c1ccc2c(c1)oc1ccccc12] |
| N28 | 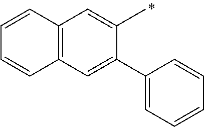 [SMILES: Cc1cc2ccccc2cc1-c1ccccc1] | 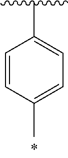 [SMILES: *c1ccc(C)cc1] | 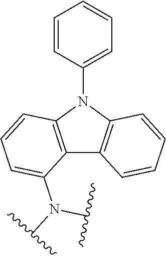 [SMILES: *N(*)c1cccc2c1c1ccccc1n2-c1ccccc1] | 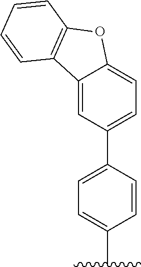 [SMILES: *c1ccc(-c2ccc3oc4ccccc4c3c2)cc1] |
| N29 | 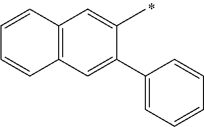 [SMILES: Cc1cc2ccccc2cc1-c1ccccc1] | 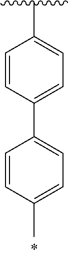 [SMILES: *c1ccc(-c2ccc(C)cc2)cc1] | 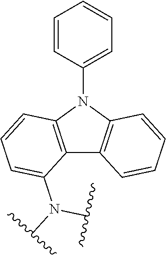 [SMILES: *N(*)c1cccc2c1c1ccccc1n2-c1ccccc1] | 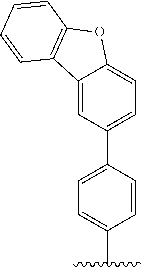 [SMILES: *c1ccc(-c2ccc3oc4ccccc4c3c2)cc1] |
| N30 | 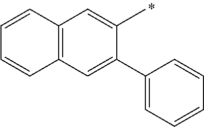 [SMILES: Cc1cc2ccccc2cc1-c1ccccc1] | 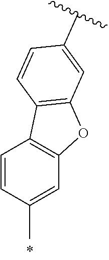 [SMILES: *c1ccc2c(c1)oc1cc(C)ccc12] | 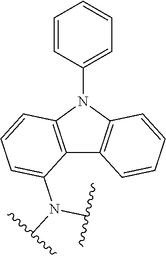 [SMILES: *N(*)c1cccc2c1c1ccccc1n2-c1ccccc1] | 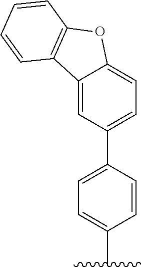 [SMILES: *c1ccc(-c2ccc3oc4ccccc4c3c2)cc1] |
| N31 | 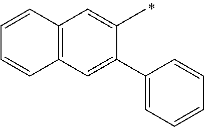 [SMILES: Cc1cc2ccccc2cc1-c1ccccc1] | 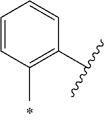 [SMILES: *c1ccccc1C] | 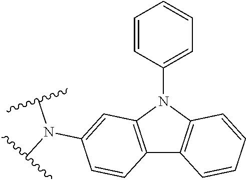 [SMILES: *N(*)c1ccc2c3ccccc3n(-c3ccccc3)c2c1] | 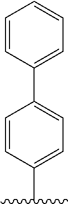 [SMILES: *c1ccc(-c2ccccc2)cc1] |
| N32 | 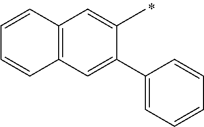 [SMILES: Cc1cc2ccccc2cc1-c1ccccc1] | 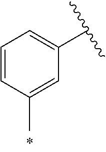 [SMILES: *c1cccc(C)c1] | 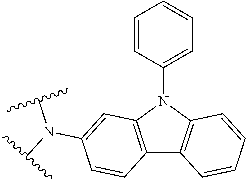 [SMILES: *N(*)c1ccc2c3ccccc3n(-c3ccccc3)c2c1] | 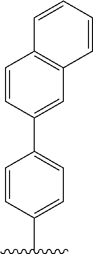 [SMILES: *c1ccc(-c2ccc3ccccc3c2)cc1] |
| N33 | 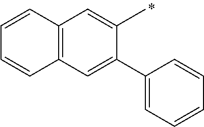 [SMILES: Cc1cc2ccccc2cc1-c1ccccc1] | 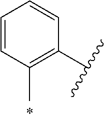 [SMILES: *c1ccccc1C] | 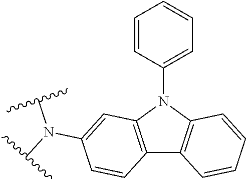 [SMILES: *N(*)c1ccc2c3ccccc3n(-c3ccccc3)c2c1] | 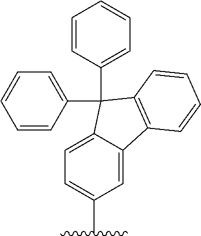 [SMILES: *c1ccc2c(c1)-c1ccccc1C2(c1ccccc1)c1ccccc1] |
| N34 | 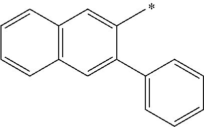 [SMILES: Cc1cc2ccccc2cc1-c1ccccc1] | 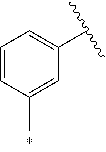 [SMILES: *c1cccc(C)c1] | 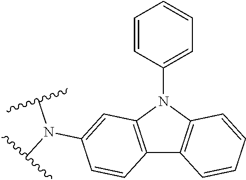 [SMILES: *N(*)c1ccc2c3ccccc3n(-c3ccccc3)c2c1] | 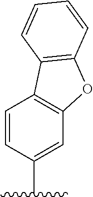 [SMILES: *c1ccc2c(c1)oc1ccccc12] |
| N35 | 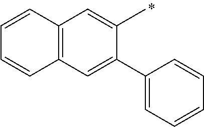 [SMILES: Cc1cc2ccccc2cc1-c1ccccc1] | 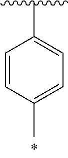 [SMILES: *c1ccc(C)cc1] | 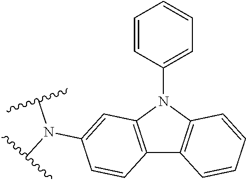 [SMILES: *N(*)c1ccc2c3ccccc3n(-c3ccccc3)c2c1] | 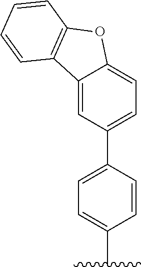 [SMILES: *c1ccc(-c2ccc3oc4ccccc4c3c2)cc1] |
| N36 | 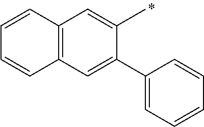 [SMILES: Cc1cc2ccccc2cc1-c1ccccc1] | 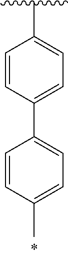 [SMILES: *c1ccc(-c2ccc(C)cc2)cc1] | 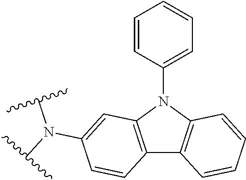 [SMILES: *N(*)c1ccc2c3ccccc3n(-c3ccccc3)c2c1] | 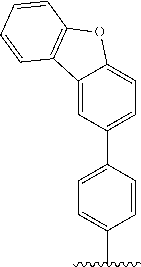 [SMILES: *c1ccc(-c2ccc3oc4ccccc4c3c2)cc1] |
| N37 | 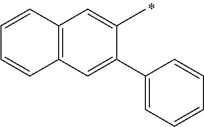 [SMILES: Cc1cc2ccccc2cc1-c1ccccc1] | 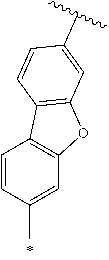 [SMILES: *c1ccc2c(c1)oc1cc(C)ccc12] | 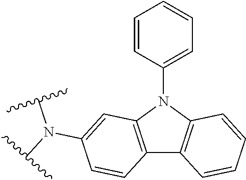 [SMILES: *N(*)c1ccc2c3ccccc3n(-c3ccccc3)c2c1] | 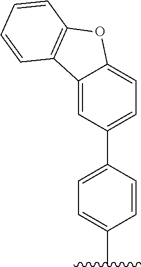 [SMILES: *c1ccc(-c2ccc3oc4ccccc4c3c2)cc1] |
| O1 | 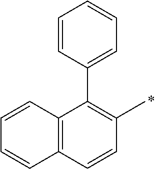 [SMILES: Cc1ccc2ccccc2c1-c1ccccc1] | 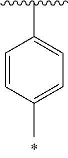 [SMILES: *c1ccc(C)cc1] | 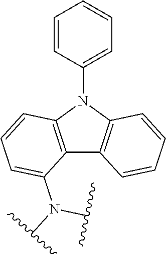 [SMILES: *N(*)c1cccc2c1c1ccccc1n2-c1ccccc1] | 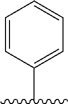 [SMILES: *c1ccccc1] |
| O2 | 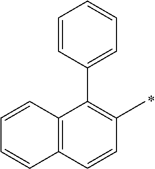 [SMILES: Cc1ccc2ccccc2c1-c1ccccc1] | 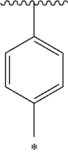 [SMILES: *c1ccc(C)cc1] | 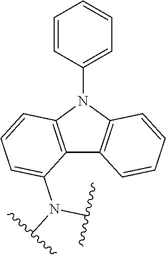 [SMILES: *N(*)c1cccc2c1c1ccccc1n2-c1ccccc1] | 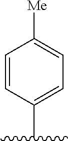 [SMILES: *c1ccc(C)cc1] |
| O3 | 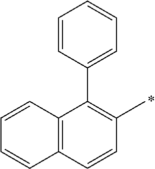 [SMILES: Cc1ccc2ccccc2c1-c1ccccc1] | 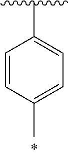 [SMILES: *c1ccc(C)cc1] | 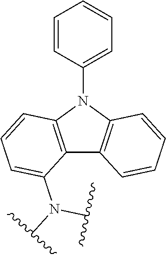 [SMILES: *N(*)c1cccc2c1c1ccccc1n2-c1ccccc1] | 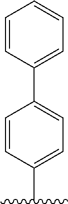 [SMILES: *c1ccc(-c2ccccc2)cc1] |
| O4 | 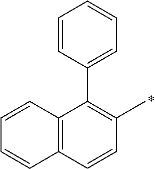 [SMILES: Cc1ccc2ccccc2c1-c1ccccc1] | 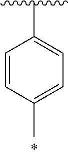 [SMILES: *c1ccc(C)cc1] | 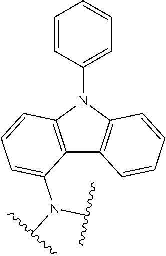 [SMILES: *N(*)c1cccc2c1c1ccccc1n2-c1ccccc1] | 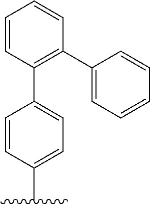 [SMILES: *c1ccc(-c2ccccc2-c2ccccc2)cc1] |
| O5 | 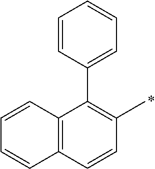 [SMILES: Cc1ccc2ccccc2c1-c1ccccc1] | 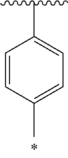 [SMILES: *c1ccc(C)cc1] | 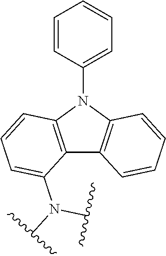 [SMILES: *N(*)c1cccc2c1c1ccccc1n2-c1ccccc1] | 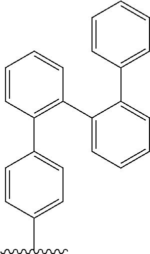 [SMILES: *c1ccc(-c2ccccc2-c2ccccc2-c2ccccc2)cc1] |
| O6 | 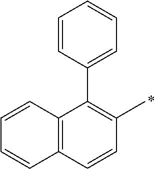 [SMILES: Cc1ccc2ccccc2c1-c1ccccc1] | 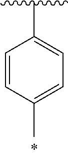 [SMILES: *c1ccc(C)cc1] | 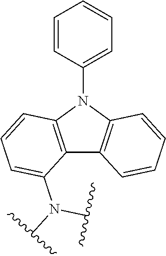 [SMILES: *N(*)c1cccc2c1c1ccccc1n2-c1ccccc1] | 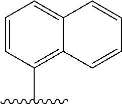 [SMILES: *c1cccc2ccccc12] |
| O7 | 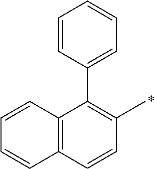 [SMILES: Cc1ccc2ccccc2c1-c1ccccc1] | 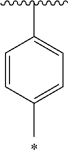 [SMILES: *c1ccc(C)cc1] | 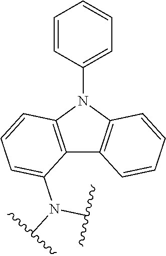 [SMILES: *N(*)c1cccc2c1c1ccccc1n2-c1ccccc1] | 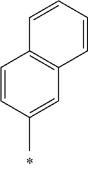 |
| O8 | 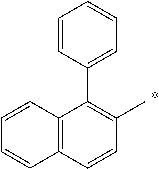 [SMILES: Cc1ccc2ccccc2c1-c1ccccc1] | 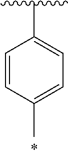 [SMILES: *c1ccc(C)cc1] | 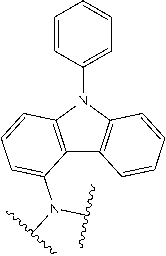 [SMILES: *N(*)c1cccc2c1c1ccccc1n2-c1ccccc1] | 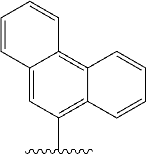 [SMILES: *c1cc2ccccc2c2ccccc12] |
| O9 | 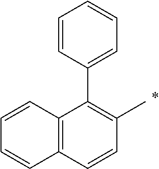 [SMILES: Cc1ccc2ccccc2c1-c1ccccc1] | 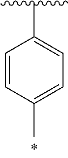 [SMILES: *c1ccc(C)cc1] | 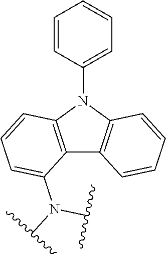 [SMILES: *N(*)c1cccc2c1c1ccccc1n2-c1ccccc1] | 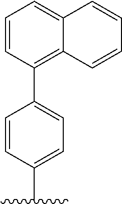 [SMILES: *c1ccc(-c2cccc3ccccc23)cc1] |
| O10 | 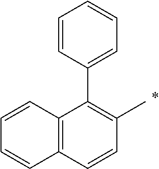 [SMILES: Cc1ccc2ccccc2c1-c1ccccc1] | 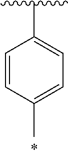 [SMILES: *c1ccc(C)cc1] | 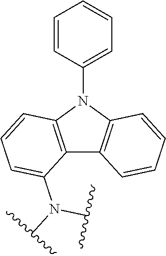 [SMILES: *N(*)c1cccc2c1c1ccccc1n2-c1ccccc1] | 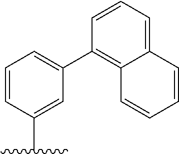 [SMILES: *c1cccc(-c2cccc3ccccc23)c1] |
| O11 | 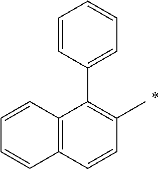 [SMILES: Cc1ccc2ccccc2c1-c1ccccc1] | 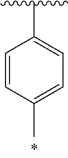 [SMILES: *c1ccc(C)cc1] | 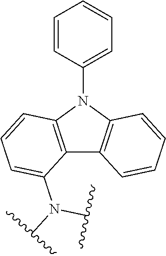 [SMILES: *N(*)c1cccc2c1c1ccccc1n2-c1ccccc1] | 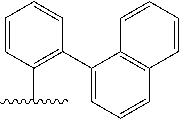 [SMILES: *c1ccccc1-c1cccc2ccccc12] |
| O12 | 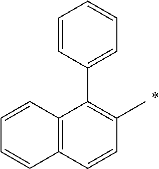 [SMILES: Cc1ccc2ccccc2c1-c1ccccc1] | 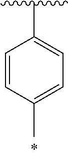 [SMILES: *c1ccc(C)cc1] | 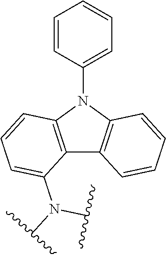 [SMILES: *N(*)c1cccc2c1c1ccccc1n2-c1ccccc1] | 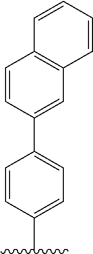 [SMILES: *c1ccc(-c2ccc3ccccc3c2)cc1] |
| O13 | 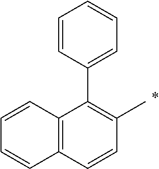 [SMILES: Cc1ccc2ccccc2c1-c1ccccc1] | 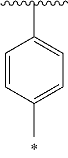 [SMILES: *c1ccc(C)cc1] | 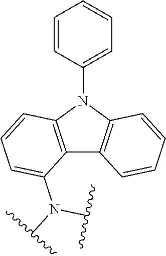 [SMILES: *N(*)c1cccc2c1c1ccccc1n2-c1ccccc1] | 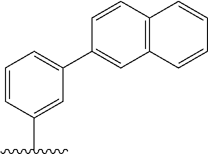 [SMILES: *c1cccc(-c2ccc3ccccc3c2)c1] |
| O14 | 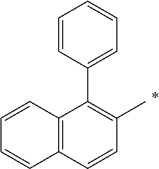 [SMILES: Cc1ccc2ccccc2c1-c1ccccc1] | 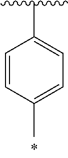 [SMILES: *c1ccc(C)cc1] | 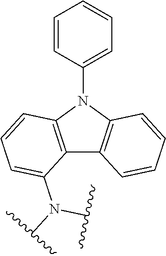 [SMILES: *N(*)c1cccc2c1c1ccccc1n2-c1ccccc1] | 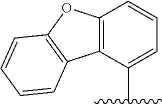 [SMILES: *c1cccc2oc3ccccc3c12] |
| O15 | 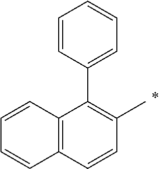 [SMILES: Cc1ccc2ccccc2c1-c1ccccc1] | 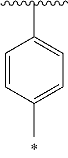 [SMILES: *c1ccc(C)cc1] | 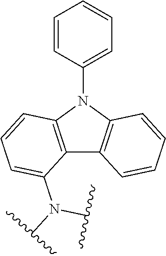 [SMILES: *N(*)c1cccc2c1c1ccccc1n2-c1ccccc1] | 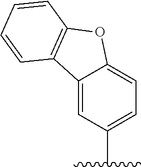 [SMILES: *c1ccc2oc3ccccc3c2c1] |
| O16 | 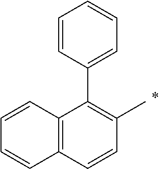 [SMILES: Cc1ccc2ccccc2c1-c1ccccc1] | 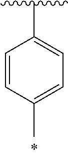 [SMILES: *c1ccc(C)cc1] | 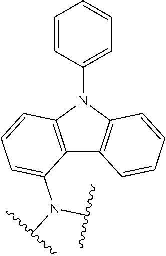 [SMILES: *N(*)c1cccc2c1c1ccccc1n2-c1ccccc1] | 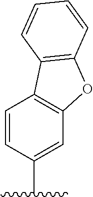 [SMILES: *c1ccc2c(c1)oc1ccccc12] |
| O17 | 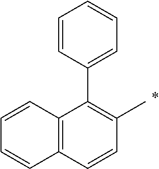 [SMILES: Cc1ccc2ccccc2c1-c1ccccc1] | 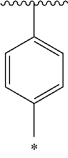 [SMILES: *c1ccc(C)cc1] | 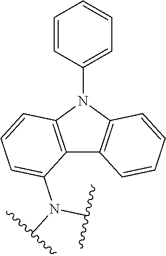 [SMILES: *N(*)c1cccc2c1c1ccccc1n2-c1ccccc1] | 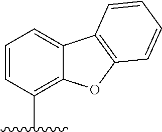 [SMILES: *c1cccc2c1oc1ccccc12] |
| O18 | 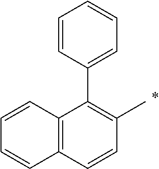 [SMILES: Cc1ccc2ccccc2c1-c1ccccc1] | 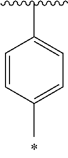 [SMILES: *c1ccc(C)cc1] | 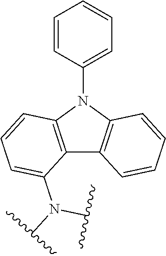 [SMILES: *N(*)c1cccc2c1c1ccccc1n2-c1ccccc1] | 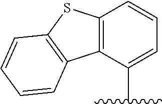 [SMILES: *c1cccc2sc3ccccc3c12] |
| O19 | 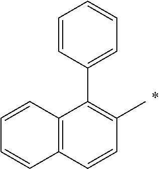 [SMILES: Cc1ccc2ccccc2c1-c1ccccc1] | 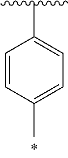 [SMILES: *c1ccc(C)cc1] | 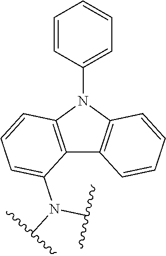 [SMILES: *N(*)c1cccc2c1c1ccccc1n2-c1ccccc1] | 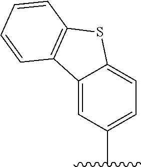 [SMILES: *c1ccc2sc3ccccc3c2c1] |
| O20 | 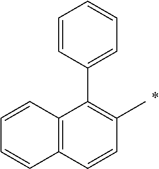 [SMILES: Cc1ccc2ccccc2c1-c1ccccc1] | 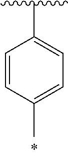 [SMILES: *c1ccc(C)cc1] | 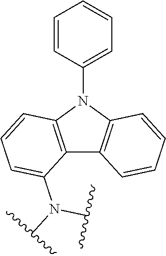 [SMILES: *N(*)c1cccc2c1c1ccccc1n2-c1ccccc1] | 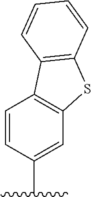 [SMILES: *c1ccc2c(c1)sc1ccccc12] |
| O21 | 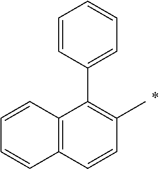 [SMILES: Cc1ccc2ccccc2c1-c1ccccc1] | 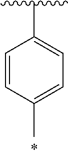 [SMILES: *c1ccc(C)cc1] | 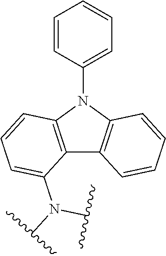 [SMILES: *N(*)c1cccc2c1c1ccccc1n2-c1ccccc1] | 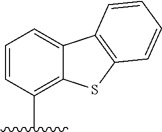 [SMILES: *c1cccc2c1sc1ccccc12] |
| O22 | 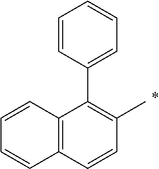 [SMILES: Cc1ccc2ccccc2c1-c1ccccc1] | 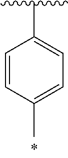 [SMILES: *c1ccc(C)cc1] | 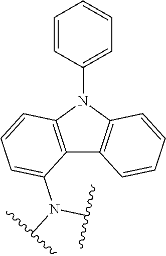 [SMILES: *N(*)c1cccc2c1c1ccccc1n2-c1ccccc1] | 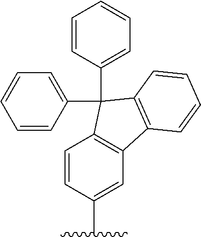 [SMILES: *c1ccc2c(c1)-c1ccccc1C2(c1ccccc1)c1ccccc1] |
| O23 | 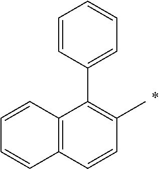 [SMILES: Cc1ccc2ccccc2c1-c1ccccc1] | 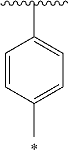 [SMILES: *c1ccc(C)cc1] | 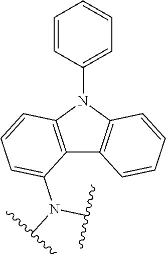 [SMILES: *N(*)c1cccc2c1c1ccccc1n2-c1ccccc1] | 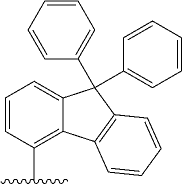 [SMILES: *c1cccc2c1-c1ccccc1C2(c1ccccc1)c1ccccc1] |
| O24 | 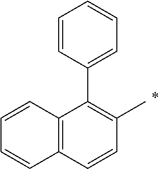 [SMILES: Cc1ccc2ccccc2c1-c1ccccc1] | 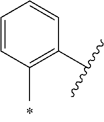 [SMILES: *c1ccccc1C] | 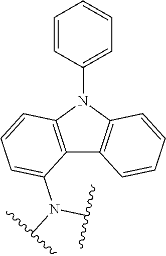 [SMILES: *N(*)c1cccc2c1c1ccccc1n2-c1ccccc1] | 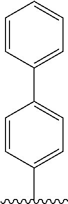 [SMILES: *c1ccc(-c2ccccc2)cc1] |
| O25 | 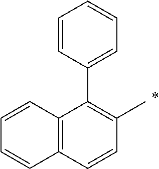 [SMILES: Cc1ccc2ccccc2c1-c1ccccc1] | 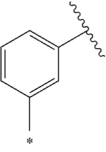 [SMILES: *c1cccc(C)c1] | 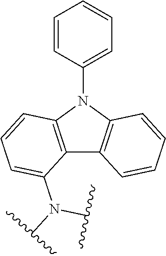 [SMILES: *N(*)c1cccc2c1c1ccccc1n2-c1ccccc1] | 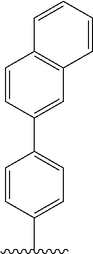 [SMILES: *c1ccc(-c2ccc3ccccc3c2)cc1] |
| O26 | 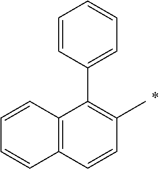 [SMILES: Cc1ccc2ccccc2c1-c1ccccc1] | 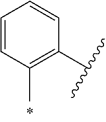 [SMILES: *c1ccccc1C] | 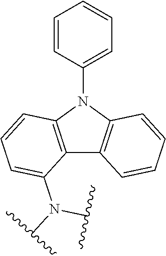 [SMILES: *N(*)c1cccc2c1c1ccccc1n2-c1ccccc1] | 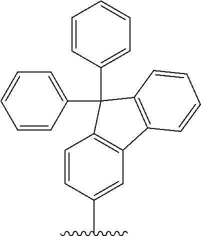 [SMILES: *c1ccc2c(c1)-c1ccccc1C2(c1ccccc1)c1ccccc1] |
| O27 | 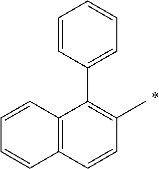 [SMILES: Cc1ccc2ccccc2c1-c1ccccc1] | 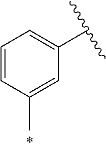 [SMILES: *c1cccc(C)c1] | 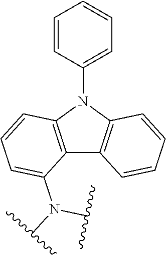 [SMILES: *N(*)c1cccc2c1c1ccccc1n2-c1ccccc1] | 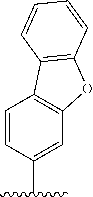 [SMILES: *c1ccc2c(c1)oc1ccccc12] |
| O28 | 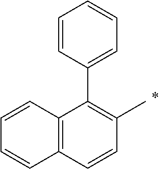 [SMILES: Cc1ccc2ccccc2c1-c1ccccc1] | 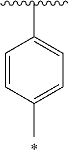 [SMILES: *c1ccc(C)cc1] | 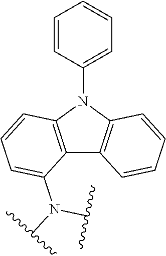 [SMILES: *N(*)c1cccc2c1c1ccccc1n2-c1ccccc1] | 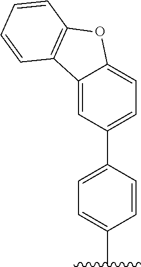 [SMILES: *c1ccc(-c2ccc3oc4ccccc4c3c2)cc1] |
| O29 | 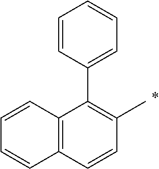 [SMILES: Cc1ccc2ccccc2c1-c1ccccc1] | 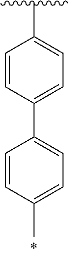 [SMILES: *c1ccc(-c2ccc(C)cc2)cc1] | 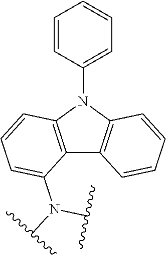 [SMILES: *N(*)c1cccc2c1c1ccccc1n2-c1ccccc1] | 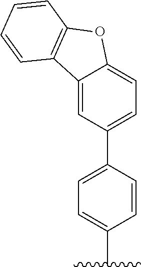 [SMILES: *c1ccc(-c2ccc3oc4ccccc4c3c2)cc1] |
| O30 | 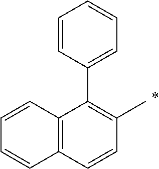 [SMILES: Cc1ccc2ccccc2c1-c1ccccc1] | 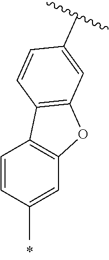 [SMILES: *c1ccc2c(c1)oc1cc(C)ccc12] | 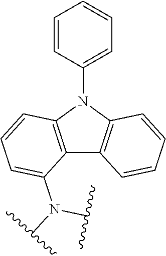 [SMILES: *N(*)c1cccc2c1c1ccccc1n2-c1ccccc1] | 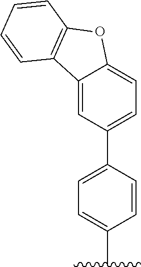 [SMILES: *c1ccc(-c2ccc3oc4ccccc4c3c2)cc1] |
| O31 | 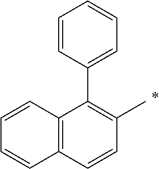 [SMILES: Cc1ccc2ccccc2c1-c1ccccc1] | 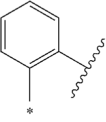 [SMILES: *c1ccccc1C] | 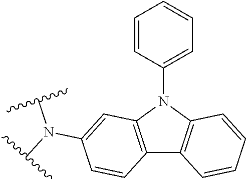 [SMILES: *N(*)c1ccc2c3ccccc3n(-c3ccccc3)c2c1] | 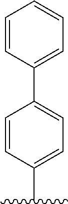 [SMILES: *c1ccc(-c2ccccc2)cc1] |
| O32 | 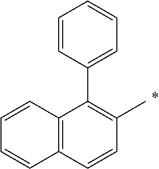 [SMILES: Cc1ccc2ccccc2c1-c1ccccc1] | 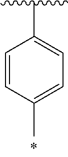 [SMILES: *c1ccc(C)cc1] | 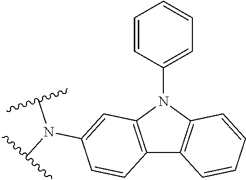 [SMILES: *N(*)c1ccc2c3ccccc3n(-c3ccccc3)c2c1] | 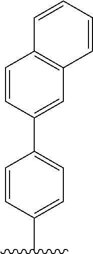 [SMILES: *c1ccc(-c2ccc3ccccc3c2)cc1] |
| O33 | 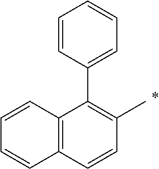 [SMILES: Cc1ccc2ccccc2c1-c1ccccc1] | 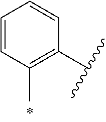 [SMILES: *c1ccccc1C] | 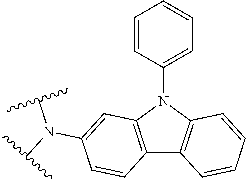 [SMILES: *N(*)c1ccc2c3ccccc3n(-c3ccccc3)c2c1] | 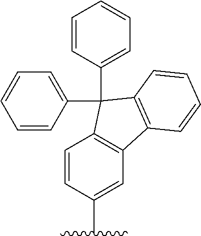 [SMILES: *c1ccc2c(c1)-c1ccccc1C2(c1ccccc1)c1ccccc1] |
| O34 | 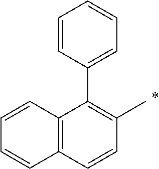 [SMILES: Cc1ccc2ccccc2c1-c1ccccc1] | 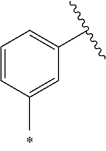 [SMILES: *c1cccc(C)c1] | 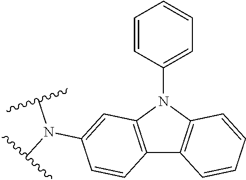 [SMILES: *N(*)c1ccc2c3ccccc3n(-c3ccccc3)c2c1] | 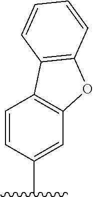 [SMILES: *c1ccc2c(c1)oc1ccccc12] |
| O35 | 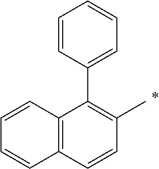 [SMILES: Cc1ccc2ccccc2c1-c1ccccc1] | 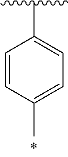 [SMILES: *c1ccc(C)cc1] | 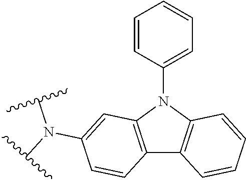 [SMILES: *N(*)c1ccc2c3ccccc3n(-c3ccccc3)c2c1] | 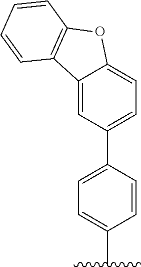 [SMILES: *c1ccc(-c2ccc3oc4ccccc4c3c2)cc1] |
| O36 | 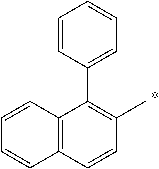 [SMILES: Cc1ccc2ccccc2c1-c1ccccc1] | 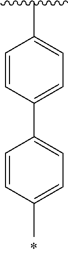 [SMILES: *c1ccc(-c2ccc(C)cc2)cc1] | 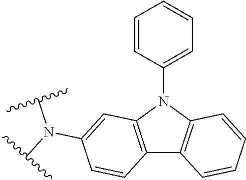 [SMILES: *N(*)c1ccc2c3ccccc3n(-c3ccccc3)c2c1] | 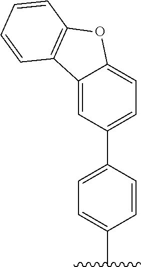 [SMILES: *c1ccc(-c2ccc3oc4ccccc4c3c2)cc1] |
| O37 | 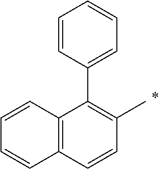 [SMILES: Cc1ccc2ccccc2c1-c1ccccc1] | 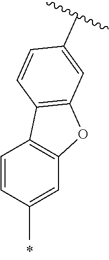 [SMILES: *c1ccc2c(c1)oc1cc(C)ccc12] | 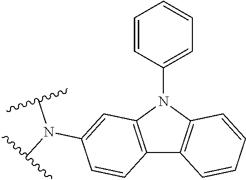 [SMILES: *N(*)c1ccc2c3ccccc3n(-c3ccccc3)c2c1] | 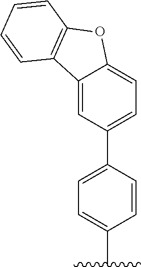 [SMILES: *c1ccc(-c2ccc3oc4ccccc4c3c2)cc1] |
| P1 | 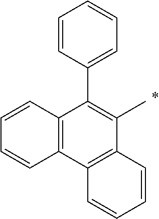 [SMILES: Cc1c(-c2ccccc2)c2ccccc2c2ccccc12] | 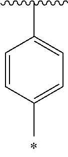 [SMILES: *c1ccc(C)cc1] | 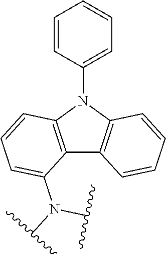 [SMILES: *N(*)c1cccc2c1c1ccccc1n2-c1ccccc1] | 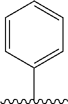 [SMILES: *c1ccccc1] |
| P2 | 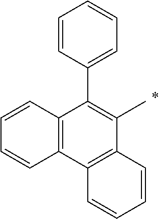 [SMILES: Cc1c(-c2ccccc2)c2ccccc2c2ccccc12] | 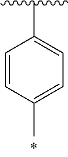 [SMILES: *c1ccc(C)cc1] | 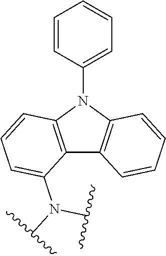 [SMILES: *N(*)c1cccc2c1c1ccccc1n2-c1ccccc1] | 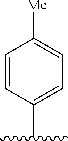 [SMILES: *c1ccc(C)cc1] |
| P3 | 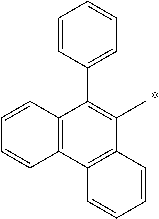 [SMILES: Cc1c(-c2ccccc2)c2ccccc2c2ccccc12] | 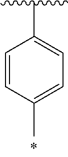 [SMILES: *c1ccc(C)cc1] | 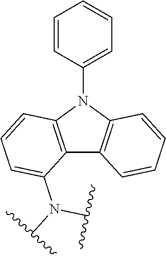 [SMILES: *N(*)c1cccc2c1c1ccccc1n2-c1ccccc1] | 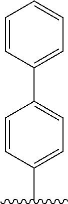 [SMILES: *c1ccc(-c2ccccc2)cc1] |
| P4 | 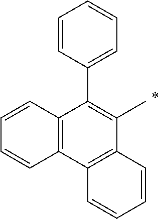 [SMILES: Cc1c(-c2ccccc2)c2ccccc2c2ccccc12] | 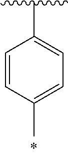 [SMILES: *c1ccc(C)cc1] | 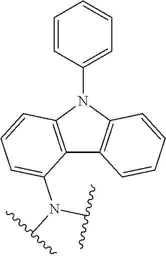 [SMILES: *N(*)c1cccc2c1c1ccccc1n2-c1ccccc1] | 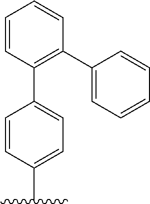 [SMILES: *c1ccc(-c2ccccc2-c2ccccc2)cc1] |
| P5 | 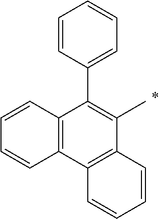 [SMILES: Cc1c(-c2ccccc2)c2ccccc2c2ccccc12] | 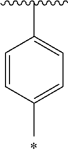 [SMILES: *c1ccc(C)cc1] | 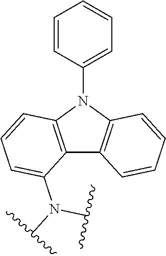 [SMILES: *N(*)c1cccc2c1c1ccccc1n2-c1ccccc1] | 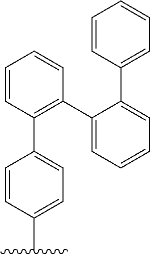 [SMILES: *c1ccc(-c2ccccc2-c2ccccc2-c2ccccc2)cc1] |
| P6 | 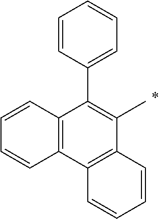 [SMILES: Cc1c(-c2ccccc2)c2ccccc2c2ccccc12] | 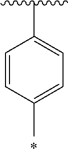 [SMILES: *c1ccc(C)cc1] | 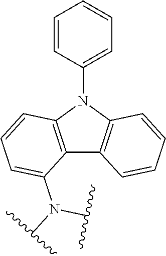 [SMILES: *N(*)c1cccc2c1c1ccccc1n2-c1ccccc1] | 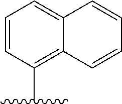 [SMILES: *c1cccc2ccccc12] |
| P7 | 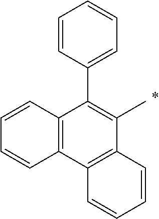 [SMILES: Cc1c(-c2ccccc2)c2ccccc2c2ccccc12] | 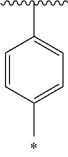 [SMILES: *c1ccc(C)cc1] | 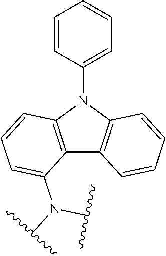 [SMILES: *N(*)c1cccc2c1c1ccccc1n2-c1ccccc1] | 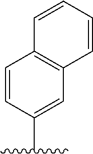 [SMILES: *c1ccc2ccccc2c1] |
| P8 | 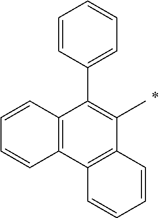 [SMILES: Cc1c(-c2ccccc2)c2ccccc2c2ccccc12] | 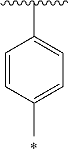 [SMILES: *c1ccc(C)cc1] | 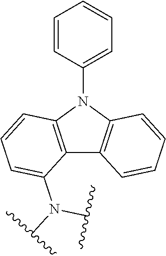 [SMILES: *N(*)c1cccc2c1c1ccccc1n2-c1ccccc1] | 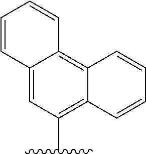 [SMILES: *c1cc2ccccc2c2ccccc12] |
| P9 | 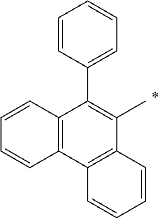 [SMILES: Cc1c(-c2ccccc2)c2ccccc2c2ccccc12] | 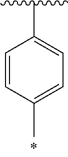 [SMILES: *c1ccc(C)cc1] | 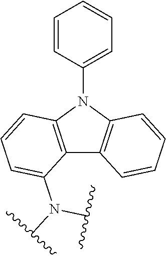 [SMILES: *N(*)c1cccc2c1c1ccccc1n2-c1ccccc1] | 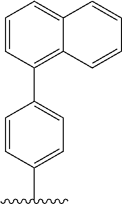 [SMILES: *c1ccc(-c2cccc3ccccc23)cc1] |
| P10 | 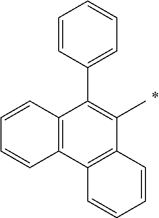 [SMILES: Cc1c(-c2ccccc2)c2ccccc2c2ccccc12] | 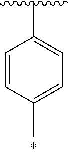 [SMILES: *c1ccc(C)cc1] | 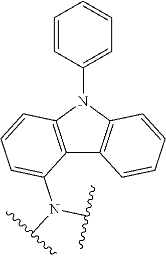 [SMILES: *N(*)c1cccc2c1c1ccccc1n2-c1ccccc1] | 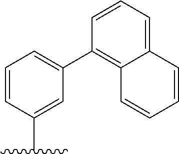 [SMILES: *c1cccc(-c2cccc3ccccc23)c1] |
| P11 | 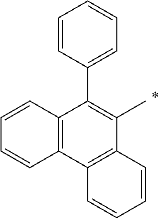 [SMILES: Cc1c(-c2ccccc2)c2ccccc2c2ccccc12] | 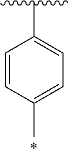 [SMILES: *c1ccc(C)cc1] | 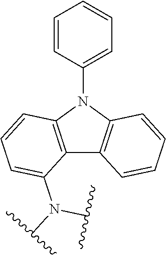 [SMILES: *N(*)c1cccc2c1c1ccccc1n2-c1ccccc1] | 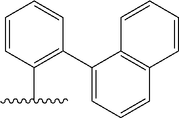 [SMILES: *c1ccccc1-c1cccc2ccccc12] |
| P12 | 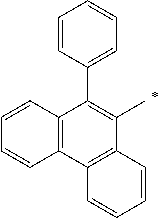 [SMILES: Cc1c(-c2ccccc2)c2ccccc2c2ccccc12] | 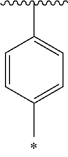 [SMILES: *c1ccc(C)cc1] | 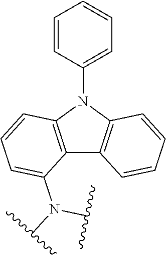 [SMILES: *N(*)c1cccc2c1c1ccccc1n2-c1ccccc1] | 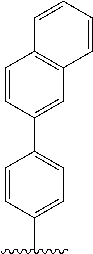 [SMILES: *c1ccc(-c2ccc3ccccc3c2)cc1] |
| P13 | 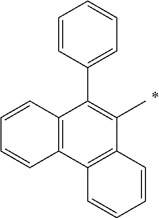 [SMILES: Cc1c(-c2ccccc2)c2ccccc2c2ccccc12] | 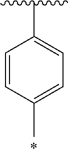 [SMILES: *c1ccc(C)cc1] | 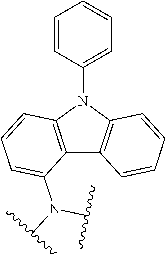 [SMILES: *N(*)c1cccc2c1c1ccccc1n2-c1ccccc1] | 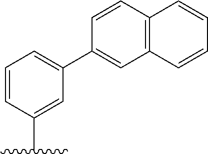 [SMILES: *c1cccc(-c2ccc3ccccc3c2)c1] |
| P14 | 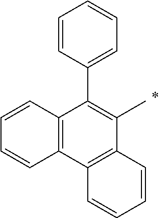 [SMILES: Cc1c(-c2ccccc2)c2ccccc2c2ccccc12] | 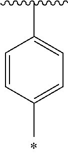 [SMILES: *c1ccc(C)cc1] | 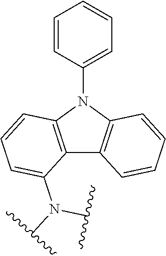 [SMILES: *N(*)c1cccc2c1c1ccccc1n2-c1ccccc1] | 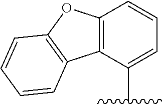 [SMILES: *c1cccc2oc3ccccc3c12] |
| P15 | 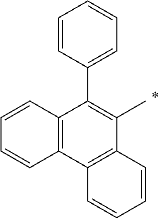 [SMILES: Cc1c(-c2ccccc2)c2ccccc2c2ccccc12] | 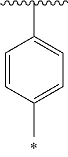 [SMILES: *c1ccc(C)cc1] | 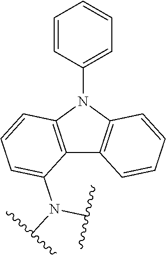 [SMILES: *N(*)c1cccc2c1c1ccccc1n2-c1ccccc1] | 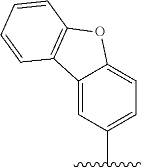 [SMILES: *c1ccc2oc3ccccc3c2c1] |
| P16 | 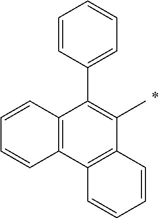 [SMILES: Cc1c(-c2ccccc2)c2ccccc2c2ccccc12] | 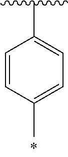 [SMILES: *c1ccc(C)cc1] | 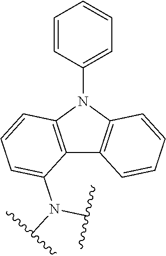 [SMILES: *N(*)c1cccc2c1c1ccccc1n2-c1ccccc1] | 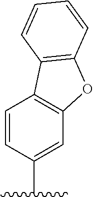 [SMILES: *c1ccc2c(c1)oc1ccccc12] |
| P17 | 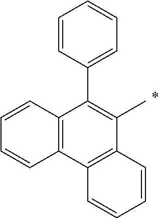 [SMILES: Cc1c(-c2ccccc2)c2ccccc2c2ccccc12] | 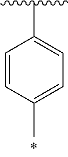 [SMILES: *c1ccc(C)cc1] | 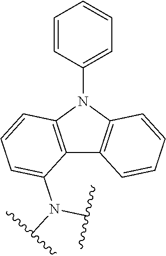 [SMILES: *N(*)c1cccc2c1c1ccccc1n2-c1ccccc1] | 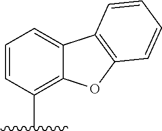 [SMILES: *c1cccc2c1oc1ccccc12] |
| P18 | 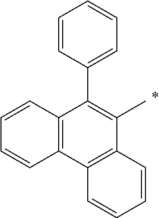 [SMILES: Cc1c(-c2ccccc2)c2ccccc2c2ccccc12] | 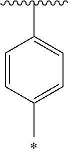 [SMILES: *c1ccc(C)cc1] | 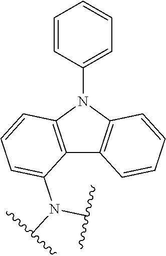 [SMILES: *N(*)c1cccc2c1c1ccccc1n2-c1ccccc1] | 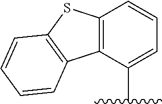 [SMILES: *c1cccc2sc3ccccc3c12] |
| P19 | 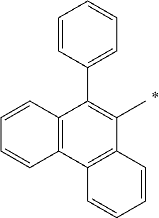 [SMILES: Cc1c(-c2ccccc2)c2ccccc2c2ccccc12] | 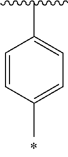 [SMILES: *c1ccc(C)cc1] | 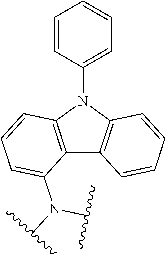 [SMILES: *N(*)c1cccc2c1c1ccccc1n2-c1ccccc1] | 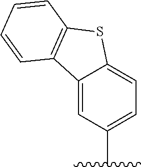 [SMILES: *c1ccc2sc3ccccc3c2c1] |
| P20 | 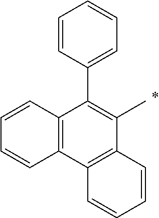 [SMILES: Cc1c(-c2ccccc2)c2ccccc2c2ccccc12] | 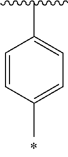 [SMILES: *c1ccc(C)cc1] | 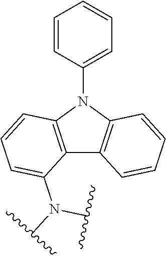 [SMILES: *N(*)c1cccc2c1c1ccccc1n2-c1ccccc1] | 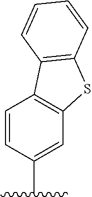 [SMILES: *c1ccc2c(c1)sc1ccccc12] |
| P21 | 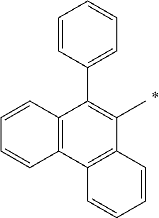 [SMILES: Cc1c(-c2ccccc2)c2ccccc2c2ccccc12] | 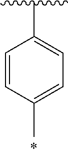 [SMILES: *c1ccc(C)cc1] | 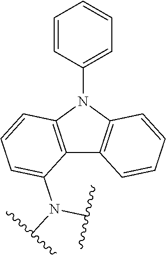 [SMILES: *N(*)c1cccc2c1c1ccccc1n2-c1ccccc1] | 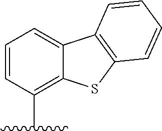 [SMILES: *c1cccc2c1sc1ccccc12] |
| P22 | 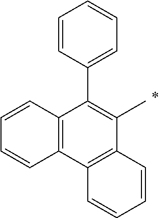 [SMILES: Cc1c(-c2ccccc2)c2ccccc2c2ccccc12] | 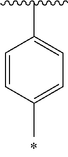 [SMILES: *c1ccc(C)cc1] | 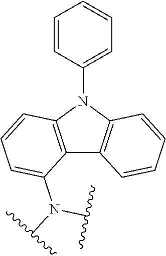 [SMILES: *N(*)c1cccc2c1c1ccccc1n2-c1ccccc1] | 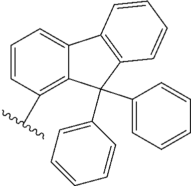 [SMILES: *c1cccc2c1C(c1ccccc1)(c1ccccc1)c1ccccc1-2] |
| P23 | 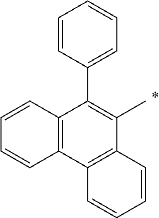 [SMILES: Cc1c(-c2ccccc2)c2ccccc2c2ccccc12] | 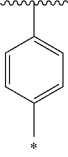 [SMILES: *c1ccc(C)cc1] | 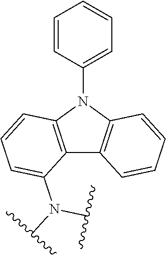 [SMILES: *N(*)c1cccc2c1c1ccccc1n2-c1ccccc1] | 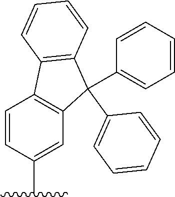 [SMILES: *c1ccc2c(c1)C(c1ccccc1)(c1ccccc1)c1ccccc1-2] |
| P24 | 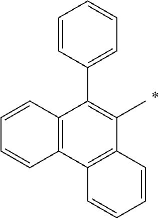 [SMILES: Cc1c(-c2ccccc2)c2ccccc2c2ccccc12] | 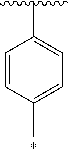 [SMILES: *c1ccc(C)cc1] | 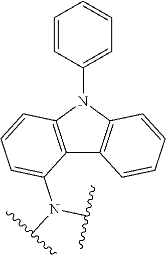 [SMILES: *N(*)c1cccc2c1c1ccccc1n2-c1ccccc1] | 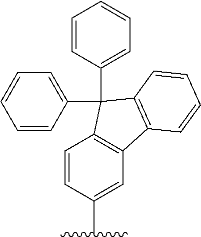 [SMILES: *c1ccc2c(c1)-c1ccccc1C2(c1ccccc1)c1ccccc1] |
| P25 | 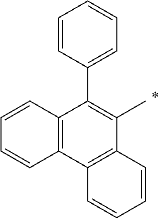 [SMILES: Cc1c(-c2ccccc2)c2ccccc2c2ccccc12] | 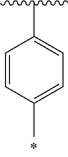 [SMILES: *c1ccc(C)cc1] | 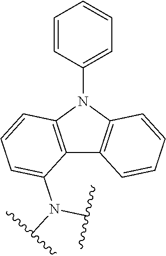 [SMILES: *N(*)c1cccc2c1c1ccccc1n2-c1ccccc1] | 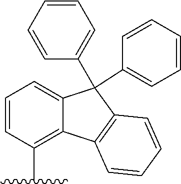 [SMILES: *c1cccc2c1-c1ccccc1C2(c1ccccc1)c1ccccc1] |
| P26 | 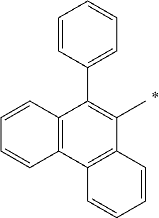 [SMILES: Cc1c(-c2ccccc2)c2ccccc2c2ccccc12] | 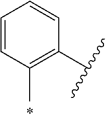 [SMILES: *c1ccccc1C] | 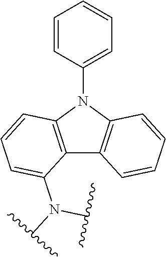 [SMILES: *N(*)c1cccc2c1c1ccccc1n2-c1ccccc1] | 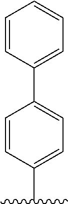 [SMILES: *c1ccc(-c2ccccc2)cc1] |
| P27 | 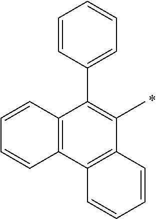 [SMILES: Cc1c(-c2ccccc2)c2ccccc2c2ccccc12] | 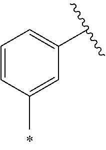 [SMILES: *c1cccc(C)c1] | 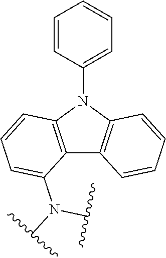 [SMILES: *N(*)c1cccc2c1c1ccccc1n2-c1ccccc1] | 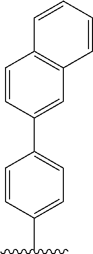 [SMILES: *c1ccc(-c2ccc3ccccc3c2)cc1] |
| P28 | 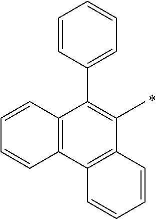 [SMILES: Cc1c(-c2ccccc2)c2ccccc2c2ccccc12] | 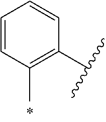 [SMILES: *c1ccccc1C] | 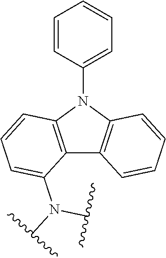 [SMILES: *N(*)c1cccc2c1c1ccccc1n2-c1ccccc1] | 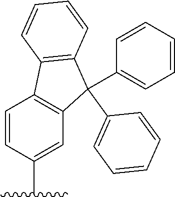 [SMILES: *c1ccc2c(c1)C(c1ccccc1)(c1ccccc1)c1ccccc1-2] |
| P29 | 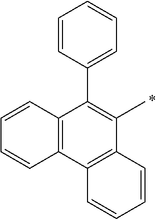 [SMILES: Cc1c(-c2ccccc2)c2ccccc2c2ccccc12] | 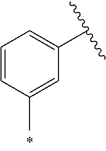 [SMILES: *c1cccc(C)c1] | 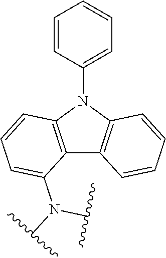 [SMILES: *N(*)c1cccc2c1c1ccccc1n2-c1ccccc1] | 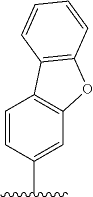 [SMILES: *c1ccc2c(c1)oc1ccccc12] |
| P30 | 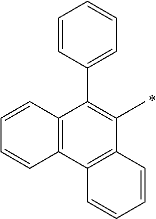 [SMILES: Cc1c(-c2ccccc2)c2ccccc2c2ccccc12] | 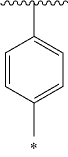 [SMILES: *c1ccc(C)cc1] | 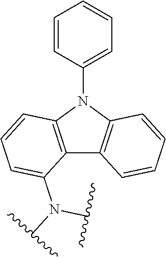 [SMILES: *N(*)c1cccc2c1c1ccccc1n2-c1ccccc1] | 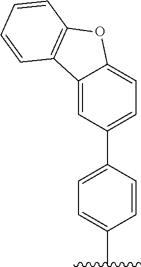 [SMILES: *c1ccc(-c2ccc3oc4ccccc4c3c2)cc1] |
| P31 | 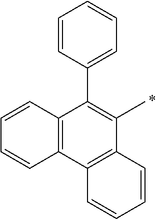 [SMILES: Cc1c(-c2ccccc2)c2ccccc2c2ccccc12] | 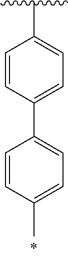 [SMILES: *c1ccc(-c2ccc(C)cc2)cc1] | 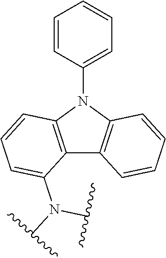 [SMILES: *N(*)c1cccc2c1c1ccccc1n2-c1ccccc1] | 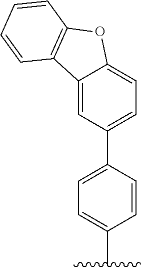 [SMILES: *c1ccc(-c2ccc3oc4ccccc4c3c2)cc1] |
| P32 | 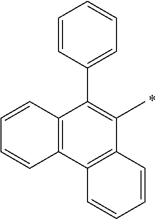 [SMILES: Cc1c(-c2ccccc2)c2ccccc2c2ccccc12] | 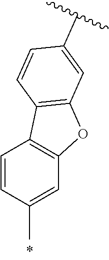 [SMILES: *c1ccc2c(c1)oc1cc(C)ccc12] | 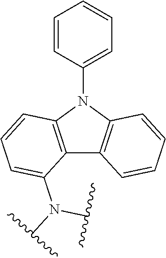 [SMILES: *N(*)c1cccc2c1c1ccccc1n2-c1ccccc1] | 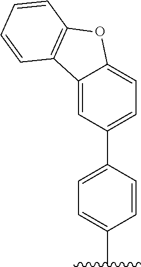 [SMILES: *c1ccc(-c2ccc3oc4ccccc4c3c2)cc1] |
| P33 | 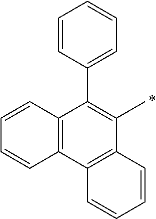 [SMILES: Cc1c(-c2ccccc2)c2ccccc2c2ccccc12] | 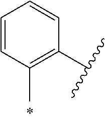 [SMILES: *c1ccccc1C] | 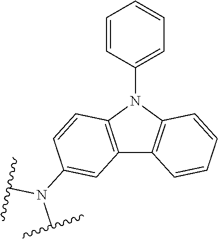 [SMILES: *N(*)c1ccc2c(c1)c1ccccc1n2-c1ccccc1] | 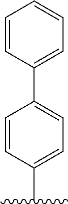 [SMILES: *c1ccc(-c2ccccc2)cc1] |
| P34 | 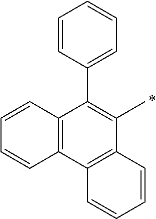 [SMILES: Cc1c(-c2ccccc2)c2ccccc2c2ccccc12] | 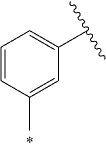 [SMILES: *c1cccc(C)c1] | 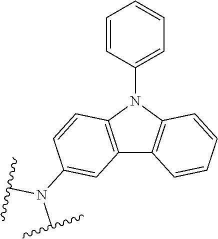 [SMILES: *N(*)c1ccc2c(c1)c1ccccc1n2-c1ccccc1] | 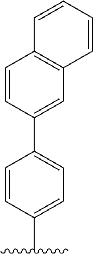 [SMILES: *c1ccc(-c2ccc3ccccc3c2)cc1] |
| P35 | 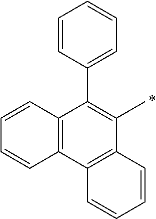 [SMILES: Cc1c(-c2ccccc2)c2ccccc2c2ccccc12] | 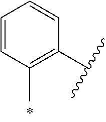 [SMILES: *c1ccccc1C] | 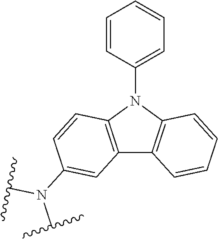 [SMILES: *N(*)c1ccc2c(c1)c1ccccc1n2-c1ccccc1] | 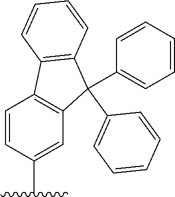 [SMILES: *c1ccc2c(c1)C(c1ccccc1)(c1ccccc1)c1ccccc1-2] |
| P36 | 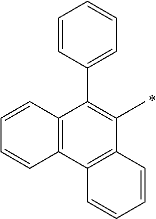 [SMILES: Cc1c(-c2ccccc2)c2ccccc2c2ccccc12] | 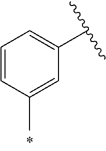 [SMILES: *c1cccc(C)c1] | 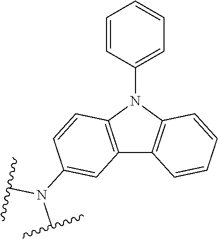 [SMILES: *N(*)c1ccc2c(c1)c1ccccc1n2-c1ccccc1] | 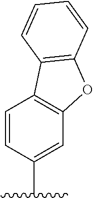 [SMILES: *c1ccc2c(c1)oc1ccccc12] |
| P37 | 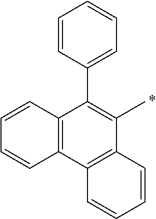 [SMILES: Cc1c(-c2ccccc2)c2ccccc2c2ccccc12] | 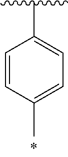 [SMILES: *c1ccc(C)cc1] | 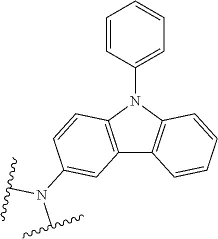 [SMILES: *N(*)c1ccc2c(c1)c1ccccc1n2-c1ccccc1] | 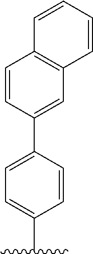 [SMILES: *c1ccc(-c2ccc3ccccc3c2)cc1] |
| P38 | 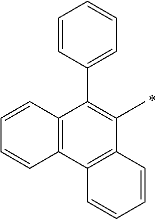 [SMILES: Cc1c(-c2ccccc2)c2ccccc2c2ccccc12] | 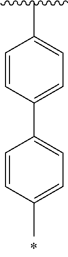 [SMILES: *c1ccc(-c2ccc(C)cc2)cc1] | 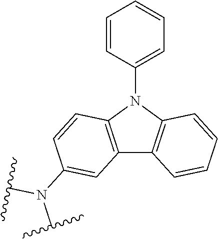 [SMILES: *N(*)c1ccc2c(c1)c1ccccc1n2-c1ccccc1] | 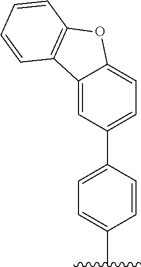 [SMILES: *c1ccc(-c2ccc3oc4ccccc4c3c2)cc1] |
| P39 | 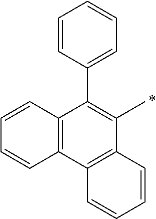 [SMILES: Cc1c(-c2ccccc2)c2ccccc2c2ccccc12] | 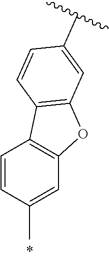 [SMILES: *c1ccc2c(c1)oc1cc(C)ccc12] | 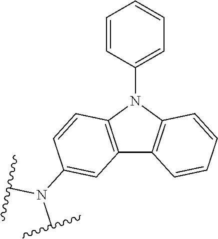 [SMILES: *N(*)c1ccc2c(c1)c1ccccc1n2-c1ccccc1] | 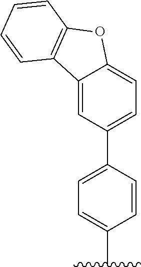 [SMILES: *c1ccc(-c2ccc3oc4ccccc4c3c2)cc1] |
| P40 | 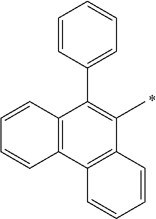 [SMILES: Cc1c(-c2ccccc2)c2ccccc2c2ccccc12] | 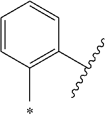 [SMILES: *c1ccccc1C] | 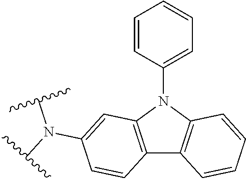 [SMILES: *N(*)c1ccc2c3ccccc3n(-c3ccccc3)c2c1] | 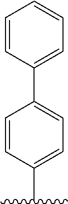 [SMILES: *c1ccc(-c2ccccc2)cc1] |
| P41 | 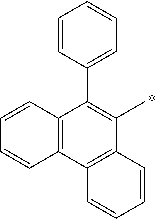 [SMILES: Cc1c(-c2ccccc2)c2ccccc2c2ccccc12] | 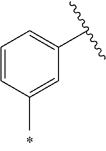 [SMILES: *c1cccc(C)c1] | 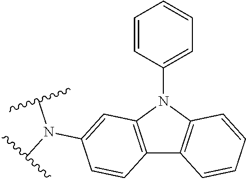 [SMILES: *N(*)c1ccc2c3ccccc3n(-c3ccccc3)c2c1] | 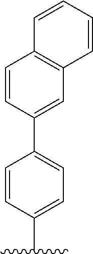 [SMILES: *c1ccc(-c2ccc3ccccc3c2)cc1] |
| P42 | 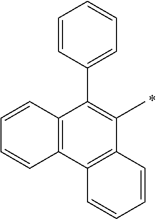 [SMILES: Cc1c(-c2ccccc2)c2ccccc2c2ccccc12] | 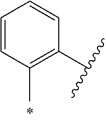 [SMILES: *c1ccccc1C] | 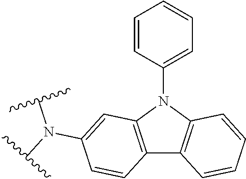 [SMILES: *N(*)c1ccc2c3ccccc3n(-c3ccccc3)c2c1] | 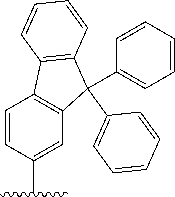 [SMILES: *c1ccc2c(c1)C(c1ccccc1)(c1ccccc1)c1ccccc1-2] |
| P43 | 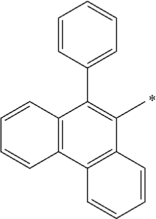 [SMILES: Cc1c(-c2ccccc2)c2ccccc2c2ccccc12] | 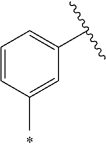 [SMILES: *c1cccc(C)c1] | 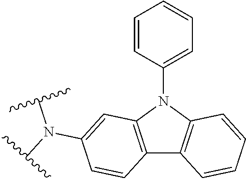 [SMILES: *N(*)c1ccc2c3ccccc3n(-c3ccccc3)c2c1] | 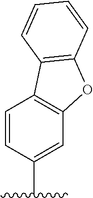 [SMILES: *c1ccc2c(c1)oc1ccccc12] |
| P44 | 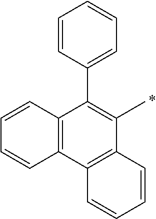 [SMILES: Cc1c(-c2ccccc2)c2ccccc2c2ccccc12] | 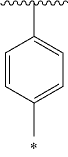 [SMILES: *c1ccc(C)cc1] | 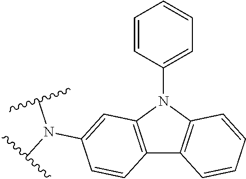 [SMILES: *N(*)c1ccc2c3ccccc3n(-c3ccccc3)c2c1] | 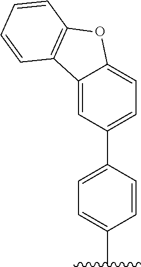 [SMILES: *c1ccc(-c2ccc3oc4ccccc4c3c2)cc1] |
| P45 | 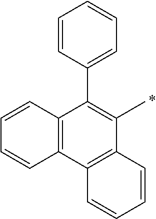 [SMILES: Cc1c(-c2ccccc2)c2ccccc2c2ccccc12] | 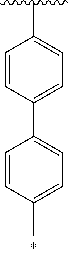 [SMILES: *c1ccc(-c2ccc(C)cc2)cc1] | 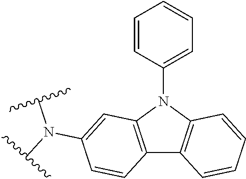 [SMILES: *N(*)c1ccc2c3ccccc3n(-c3ccccc3)c2c1] | 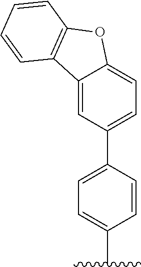 [SMILES: *c1ccc(-c2ccc3oc4ccccc4c3c2)cc1] |
| P46 | 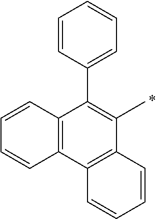 [SMILES: Cc1c(-c2ccccc2)c2ccccc2c2ccccc12] | 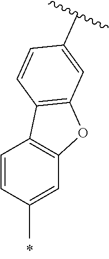 [SMILES: *c1ccc2c(c1)oc1cc(C)ccc12] | 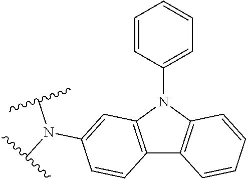 [SMILES: *N(*)c1ccc2c3ccccc3n(-c3ccccc3)c2c1] | 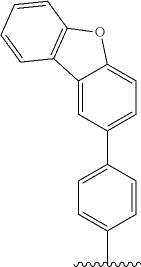 [SMILES: *c1ccc(-c2ccc3oc4ccccc4c3c2)cc1] |
| Q1 | 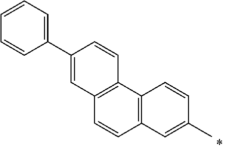 [SMILES: Cc1ccc2c(ccc3cc(-c4ccccc4)ccc32)c1] | 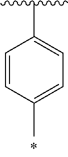 [SMILES: *c1ccc(C)cc1] | 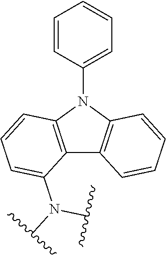 [SMILES: *N(*)c1cccc2c1c1ccccc1n2-c1ccccc1] | 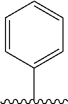 [SMILES: *c1ccccc1] |
| Q2 | 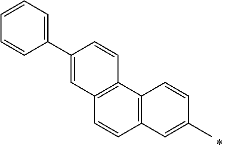 [SMILES: Cc1ccc2c(ccc3cc(-c4ccccc4)ccc32)c1] | 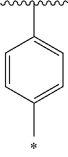 [SMILES: *c1ccc(C)cc1] | 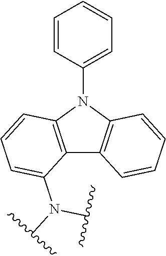 [SMILES: *N(*)c1cccc2c1c1ccccc1n2-c1ccccc1] | 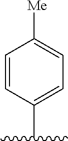 [SMILES: *c1ccc(C)cc1] |
| Q3 | 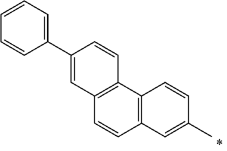 [SMILES: Cc1ccc2c(ccc3cc(-c4ccccc4)ccc32)c1] | 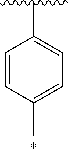 [SMILES: *c1ccc(C)cc1] | 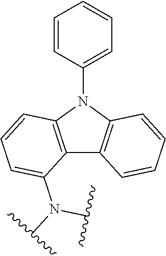 [SMILES: *N(*)c1cccc2c1c1ccccc1n2-c1ccccc1] | 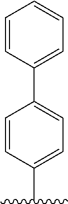 [SMILES: *c1ccc(-c2ccccc2)cc1] |
| Q4 | 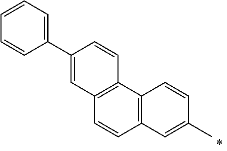 [SMILES: Cc1ccc2c(ccc3cc(-c4ccccc4)ccc32)c1] | 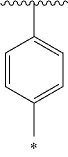 [SMILES: *c1ccc(C)cc1] | 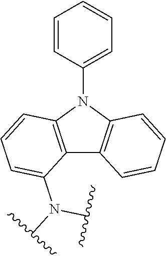 [SMILES: *N(*)c1cccc2c1c1ccccc1n2-c1ccccc1] | 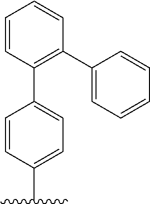 [SMILES: *c1ccc(-c2ccccc2-c2ccccc2)cc1] |
| Q5 | 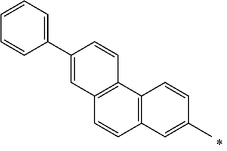 [SMILES: Cc1ccc2c(ccc3cc(-c4ccccc4)ccc32)c1] | 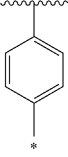 [SMILES: *c1ccc(C)cc1] | 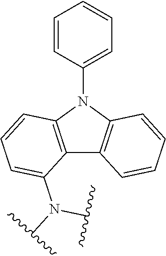 [SMILES: *N(*)c1cccc2c1c1ccccc1n2-c1ccccc1] | 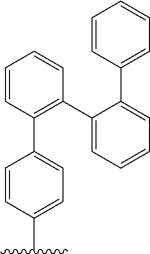 [SMILES: *c1ccc(-c2ccccc2-c2ccccc2-c2ccccc2)cc1] |
| Q6 | 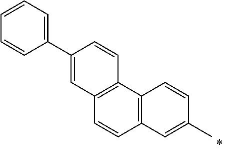 [SMILES: Cc1ccc2c(ccc3cc(-c4ccccc4)ccc32)c1] | 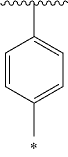 [SMILES: *c1ccc(C)cc1] | 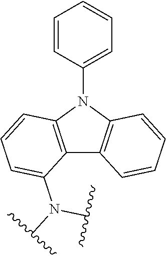 [SMILES: *N(*)c1cccc2c1c1ccccc1n2-c1ccccc1] | 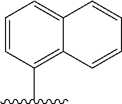 [SMILES: *c1cccc2ccccc12] |
| Q7 | 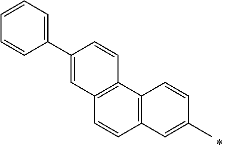 [SMILES: Cc1ccc2c(ccc3cc(-c4ccccc4)ccc32)c1] | 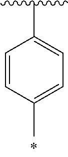 [SMILES: *c1ccc(C)cc1] | 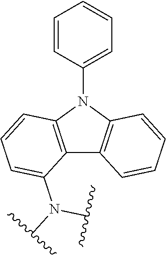 [SMILES: *N(*)c1cccc2c1c1ccccc1n2-c1ccccc1] | 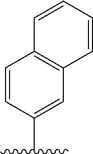 [SMILES: *c1ccc2ccccc2c1] |
| Q8 | 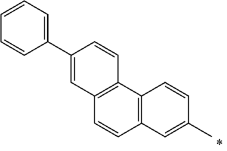 [SMILES: Cc1ccc2c(ccc3cc(-c4ccccc4)ccc32)c1] | 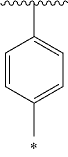 [SMILES: *c1ccc(C)cc1] | 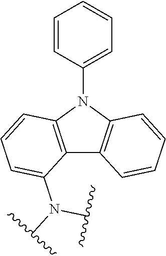 [SMILES: *N(*)c1cccc2c1c1ccccc1n2-c1ccccc1] | 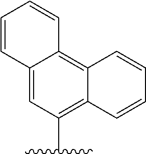 [SMILES: *c1cc2ccccc2c2ccccc12] |
| Q9 | 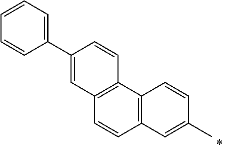 [SMILES: Cc1ccc2c(ccc3cc(-c4ccccc4)ccc32)c1] | 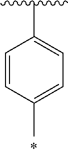 [SMILES: *c1ccc(C)cc1] | 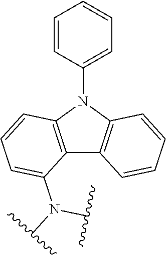 [SMILES: *N(*)c1cccc2c1c1ccccc1n2-c1ccccc1] | 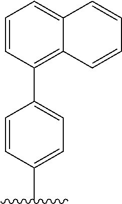 [SMILES: *c1ccc(-c2cccc3ccccc23)cc1] |
| Q10 | 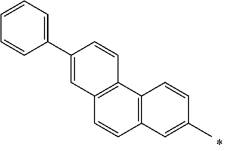 [SMILES: Cc1ccc2c(ccc3cc(-c4ccccc4)ccc32)c1] | 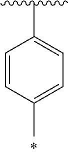 [SMILES: *c1ccc(C)cc1] | 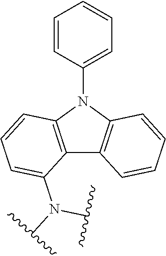 [SMILES: *N(*)c1cccc2c1c1ccccc1n2-c1ccccc1] | 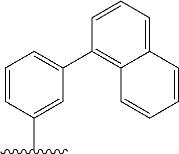 [SMILES: *c1cccc(-c2cccc3ccccc23)c1] |
| Q11 | 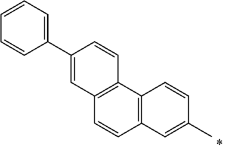 [SMILES: Cc1ccc2c(ccc3cc(-c4ccccc4)ccc32)c1] | 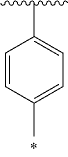 [SMILES: *c1ccc(C)cc1] | 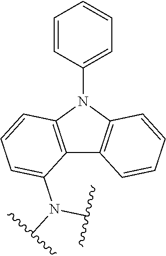 [SMILES: *N(*)c1cccc2c1c1ccccc1n2-c1ccccc1] | 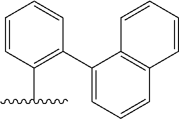 [SMILES: *c1ccccc1-c1cccc2ccccc12] |
| Q12 | 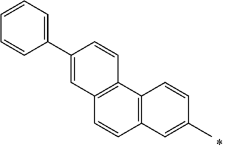 [SMILES: Cc1ccc2c(ccc3cc(-c4ccccc4)ccc32)c1] | 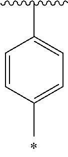 [SMILES: *c1ccc(C)cc1] | 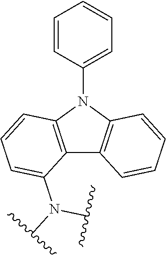 [SMILES: *N(*)c1cccc2c1c1ccccc1n2-c1ccccc1] | 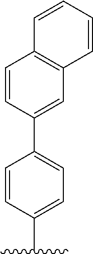 [SMILES: *c1ccc(-c2ccc3ccccc3c2)cc1] |
| Q13 | 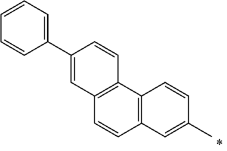 [SMILES: Cc1ccc2c(ccc3cc(-c4ccccc4)ccc32)c1] | 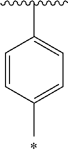 [SMILES: *c1ccc(C)cc1] | 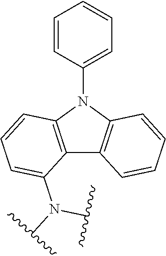 [SMILES: *N(*)c1cccc2c1c1ccccc1n2-c1ccccc1] | 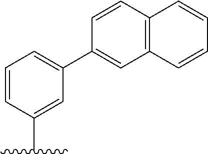 [SMILES: *c1cccc(-c2ccc3ccccc3c2)c1] |
| Q14 | 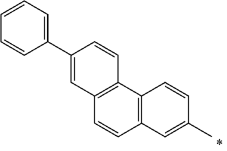 [SMILES: Cc1ccc2c(ccc3cc(-c4ccccc4)ccc32)c1] | 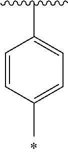 [SMILES: *c1ccc(C)cc1] | 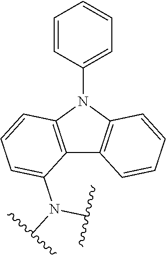 [SMILES: *N(*)c1cccc2c1c1ccccc1n2-c1ccccc1] | 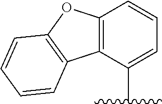 [SMILES: *c1cccc2oc3ccccc3c12] |
| Q15 | 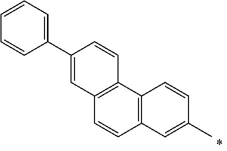 [SMILES: Cc1ccc2c(ccc3cc(-c4ccccc4)ccc32)c1] | 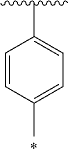 [SMILES: *c1ccc(C)cc1] | 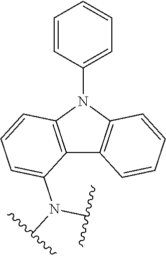 [SMILES: *N(*)c1cccc2c1c1ccccc1n2-c1ccccc1] | 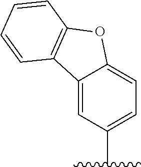 [SMILES: *c1ccc2oc3ccccc3c2c1] |
| Q16 | 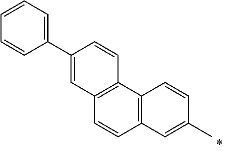 [SMILES: Cc1ccc2c(ccc3cc(-c4ccccc4)ccc32)c1] | 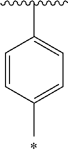 [SMILES: *c1ccc(C)cc1] | 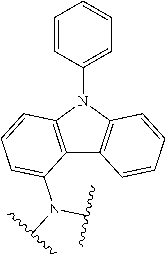 [SMILES: *N(*)c1cccc2c1c1ccccc1n2-c1ccccc1] | 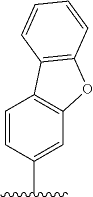 [SMILES: *c1ccc2c(c1)oc1ccccc12] |
| Q17 | 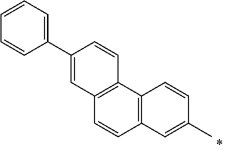 [SMILES: Cc1ccc2c(ccc3cc(-c4ccccc4)ccc32)c1] | 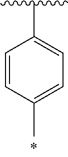 [SMILES: *c1ccc(C)cc1] | 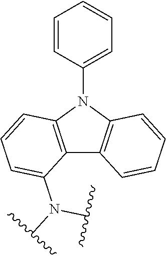 [SMILES: *N(*)c1cccc2c1c1ccccc1n2-c1ccccc1] | 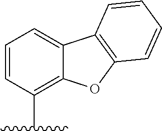 [SMILES: *c1cccc2c1oc1ccccc12] |
| Q18 | 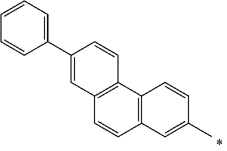 [SMILES: Cc1ccc2c(ccc3cc(-c4ccccc4)ccc32)c1] | 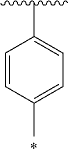 [SMILES: *c1ccc(C)cc1] | 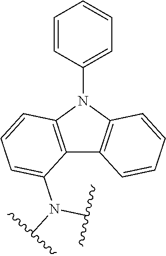 [SMILES: *N(*)c1cccc2c1c1ccccc1n2-c1ccccc1] | 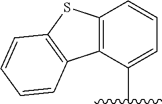 [SMILES: *c1cccc2sc3ccccc3c12] |
| Q19 | 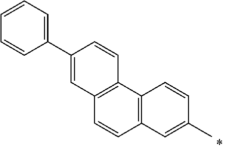 [SMILES: Cc1ccc2c(ccc3cc(-c4ccccc4)ccc32)c1] | 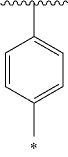 [SMILES: *c1ccc(C)cc1] | 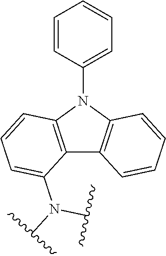 [SMILES: *N(*)c1cccc2c1c1ccccc1n2-c1ccccc1] | 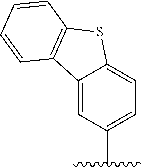 [SMILES: *c1ccc2sc3ccccc3c2c1] |
| Q20 | 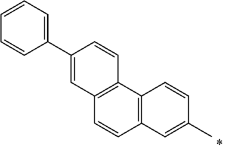 [SMILES: Cc1ccc2c(ccc3cc(-c4ccccc4)ccc32)c1] | 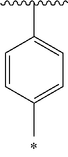 [SMILES: *c1ccc(C)cc1] | 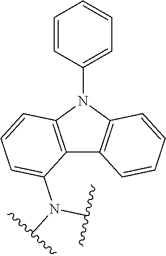 [SMILES: *N(*)c1cccc2c1c1ccccc1n2-c1ccccc1] | 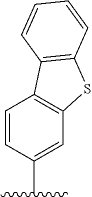 [SMILES: *c1ccc2c(c1)sc1ccccc12] |
| Q21 | 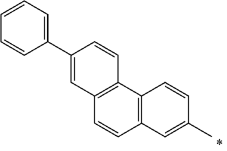 [SMILES: Cc1ccc2c(ccc3cc(-c4ccccc4)ccc32)c1] | 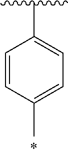 [SMILES: *c1ccc(C)cc1] | 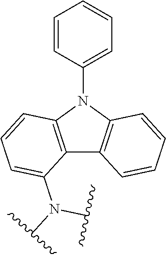 [SMILES: *N(*)c1cccc2c1c1ccccc1n2-c1ccccc1] | 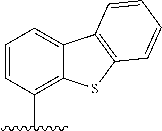 [SMILES: *c1cccc2c1sc1ccccc12] |
| Q22 | 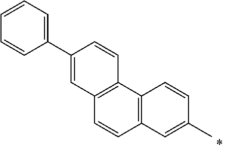 [SMILES: Cc1ccc2c(ccc3cc(-c4ccccc4)ccc32)c1] | 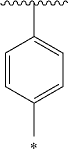 [SMILES: *c1ccc(C)cc1] | 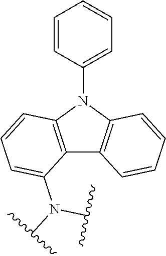 [SMILES: *N(*)c1cccc2c1c1ccccc1n2-c1ccccc1] | 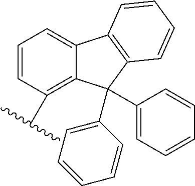 [SMILES: *c1cccc2c1C(c1ccccc1)(c1ccccc1)c1ccccc1-2] |
| Q23 | 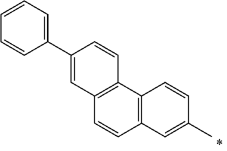 [SMILES: Cc1ccc2c(ccc3cc(-c4ccccc4)ccc32)c1] | 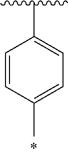 [SMILES: *c1ccc(C)cc1] | 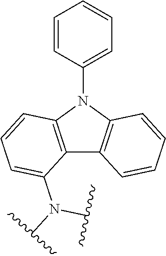 [SMILES: *N(*)c1cccc2c1c1ccccc1n2-c1ccccc1] | 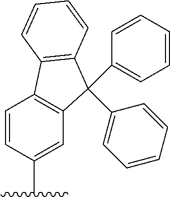 [SMILES: *c1ccc2c(c1)C(c1ccccc1)(c1ccccc1)c1ccccc1-2] |
| Q24 | 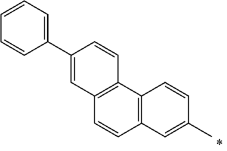 [SMILES: Cc1ccc2c(ccc3cc(-c4ccccc4)ccc32)c1] | 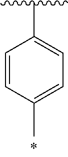 [SMILES: *c1ccc(C)cc1] | 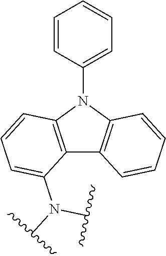 [SMILES: *N(*)c1cccc2c1c1ccccc1n2-c1ccccc1] | 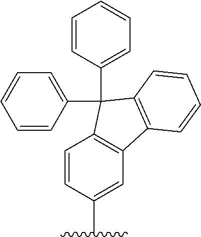 [SMILES: *c1ccc2c(c1)-c1ccccc1C2(c1ccccc1)c1ccccc1] |
| Q25 | 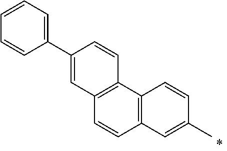 [SMILES: Cc1ccc2c(ccc3cc(-c4ccccc4)ccc32)c1] | 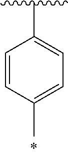 [SMILES: *c1ccc(C)cc1] | 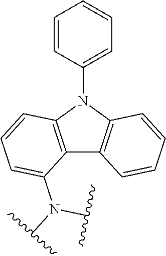 [SMILES: *N(*)c1cccc2c1c1ccccc1n2-c1ccccc1] | 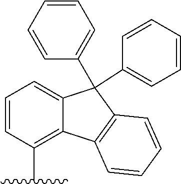 [SMILES: *c1cccc2c1-c1ccccc1C2(c1ccccc1)c1ccccc1] |
| Q26 | 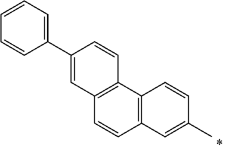 [SMILES: Cc1ccc2c(ccc3cc(-c4ccccc4)ccc32)c1] | 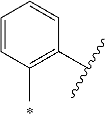 [SMILES: *c1ccccc1C] | 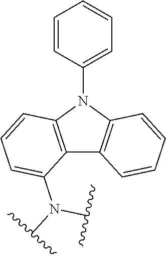 [SMILES: *N(*)c1cccc2c1c1ccccc1n2-c1ccccc1] | 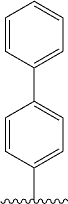 [SMILES: *c1ccc(-c2ccccc2)cc1] |
| Q27 | 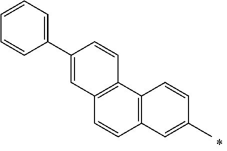 [SMILES: Cc1ccc2c(ccc3cc(-c4ccccc4)ccc32)c1] | 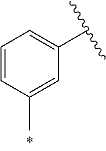 [SMILES: *c1cccc(C)c1] | 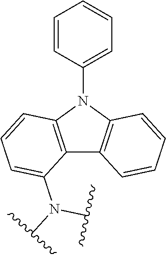 [SMILES: *N(*)c1cccc2c1c1ccccc1n2-c1ccccc1] | 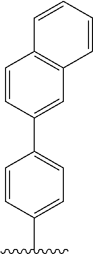 [SMILES: *c1ccc(-c2ccc3ccccc3c2)cc1] |
| Q28 | 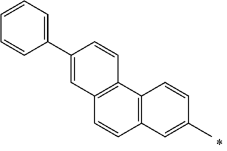 [SMILES: Cc1ccc2c(ccc3cc(-c4ccccc4)ccc32)c1] | 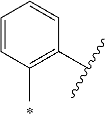 [SMILES: *c1ccccc1C] | 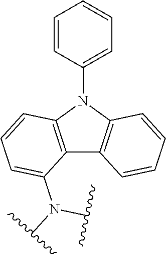 [SMILES: *N(*)c1cccc2c1c1ccccc1n2-c1ccccc1] | 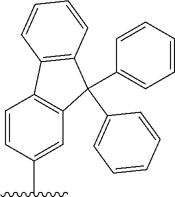 [SMILES: *c1ccc2c(c1)C(c1ccccc1)(c1ccccc1)c1ccccc1-2] |
| Q29 | 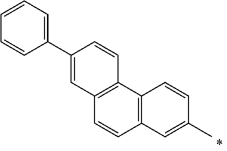 [SMILES: Cc1ccc2c(ccc3cc(-c4ccccc4)ccc32)c1] | 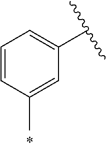 [SMILES: *c1cccc(C)c1] | 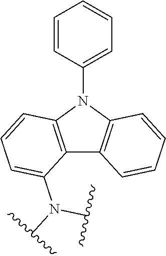 [SMILES: *N(*)c1cccc2c1c1ccccc1n2-c1ccccc1] | 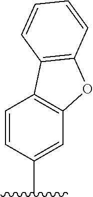 [SMILES: *c1ccc2c(c1)oc1ccccc12] |
| Q30 | 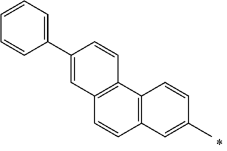 [SMILES: Cc1ccc2c(ccc3cc(-c4ccccc4)ccc32)c1] | 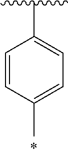 [SMILES: *c1ccc(C)cc1] | 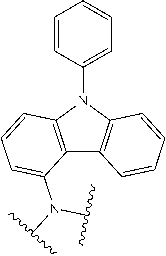 [SMILES: *N(*)c1cccc2c1c1ccccc1n2-c1ccccc1] | 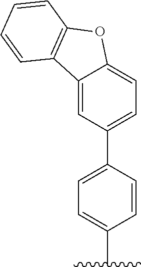 [SMILES: *c1ccc(-c2ccc3oc4ccccc4c3c2)cc1] |
| Q31 | 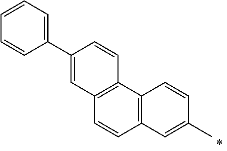 [SMILES: Cc1ccc2c(ccc3cc(-c4ccccc4)ccc32)c1] | 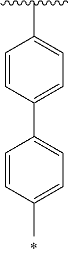 [SMILES: *c1ccc(-c2ccc(C)cc2)cc1] | 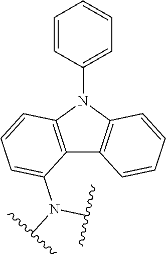 [SMILES: *N(*)c1cccc2c1c1ccccc1n2-c1ccccc1] | 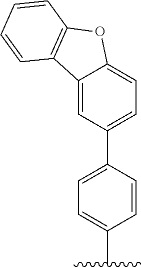 [SMILES: *c1ccc(-c2ccc3oc4ccccc4c3c2)cc1] |
| Q32 | 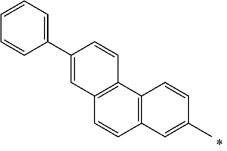 [SMILES: Cc1ccc2c(ccc3cc(-c4ccccc4)ccc32)c1] | 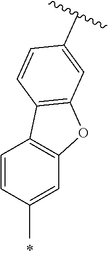 [SMILES: *c1ccc2c(c1)oc1cc(C)ccc12] | 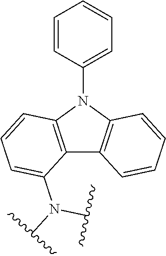 [SMILES: *N(*)c1cccc2c1c1ccccc1n2-c1ccccc1] | 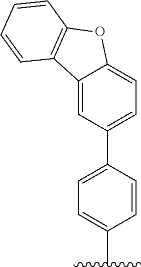 [SMILES: *c1ccc(-c2ccc3oc4ccccc4c3c2)cc1] |
| Q33 | 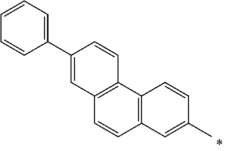 [SMILES: Cc1ccc2c(ccc3cc(-c4ccccc4)ccc32)c1] | 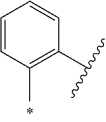 [SMILES: *c1ccccc1C] | 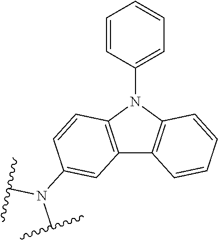 [SMILES: *N(*)c1ccc2c(c1)c1ccccc1n2-c1ccccc1] | 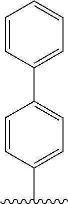 [SMILES: *c1ccc(-c2ccccc2)cc1] |
| Q34 | 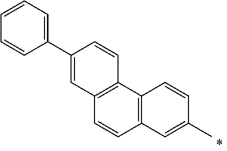 [SMILES: Cc1ccc2c(ccc3cc(-c4ccccc4)ccc32)c1] | 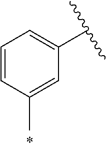 [SMILES: *c1cccc(C)c1] | 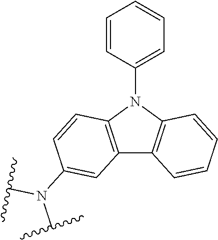 [SMILES: *N(*)c1ccc2c(c1)c1ccccc1n2-c1ccccc1] | 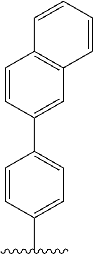 [SMILES: *c1ccc(-c2ccc3ccccc3c2)cc1] |
| Q35 | 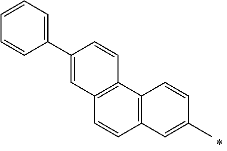 [SMILES: Cc1ccc2c(ccc3cc(-c4ccccc4)ccc32)c1] | 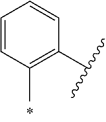 [SMILES: *c1ccccc1C] | 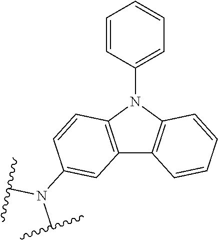 [SMILES: *N(*)c1ccc2c(c1)c1ccccc1n2-c1ccccc1] | 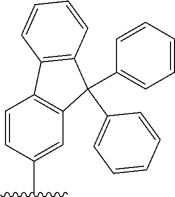 [SMILES: *c1ccc2c(c1)C(c1ccccc1)(c1ccccc1)c1ccccc1-2] |
| Q36 | 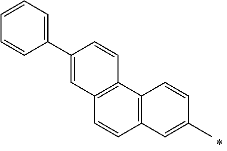 [SMILES: Cc1ccc2c(ccc3cc(-c4ccccc4)ccc32)c1] | 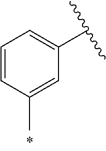 [SMILES: *c1cccc(C)c1] | 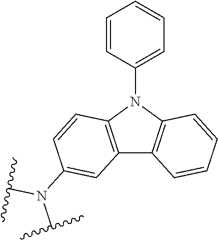 [SMILES: *N(*)c1ccc2c(c1)c1ccccc1n2-c1ccccc1] | 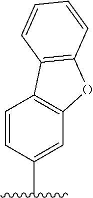 [SMILES: *c1ccc2c(c1)oc1ccccc12] |
| Q37 | 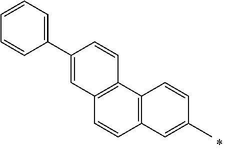 [SMILES: Cc1ccc2c(ccc3cc(-c4ccccc4)ccc32)c1] | 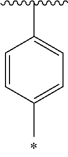 [SMILES: *c1ccc(C)cc1] | 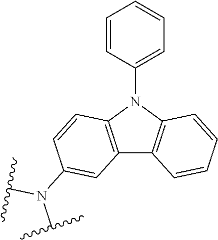 [SMILES: *N(*)c1ccc2c(c1)c1ccccc1n2-c1ccccc1] | 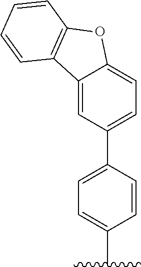 [SMILES: *c1ccc(-c2ccc3oc4ccccc4c3c2)cc1] |
| Q38 | 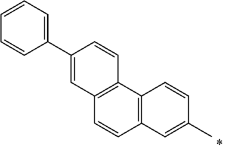 [SMILES: Cc1ccc2c(ccc3cc(-c4ccccc4)ccc32)c1] | 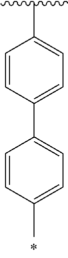 [SMILES: *c1ccc(-c2ccc(C)cc2)cc1] | 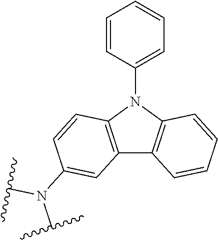 [SMILES: *N(*)c1ccc2c(c1)c1ccccc1n2-c1ccccc1] | 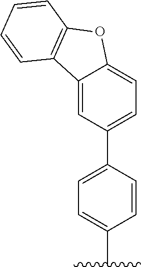 [SMILES: *c1ccc(-c2ccc3oc4ccccc4c3c2)cc1] |
| Q39 | 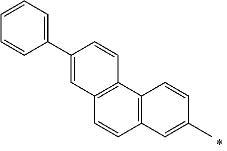 [SMILES: Cc1ccc2c(ccc3cc(-c4ccccc4)ccc32)c1] | 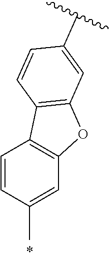 [SMILES: *c1ccc2c(c1)oc1cc(C)ccc12] | 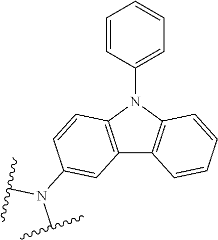 [SMILES: *N(*)c1ccc2c(c1)c1ccccc1n2-c1ccccc1] | 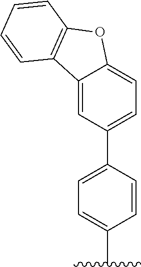 [SMILES: *c1ccc(-c2ccc3oc4ccccc4c3c2)cc1] |
| Q40 | 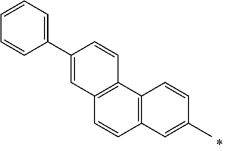 [SMILES: Cc1ccc2c(ccc3cc(-c4ccccc4)ccc32)c1] | 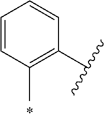 [SMILES: *c1ccccc1C] | 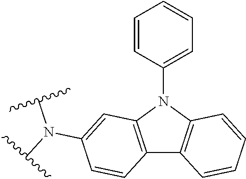 [SMILES: *N(*)c1ccc2c3ccccc3n(-c3ccccc3)c2c1] | 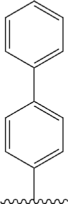 [SMILES: *c1ccc(-c2ccccc2)cc1] |
| Q41 | 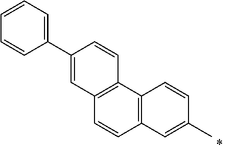 [SMILES: Cc1ccc2c(ccc3cc(-c4ccccc4)ccc32)c1] | 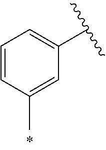 [SMILES: *c1cccc(C)c1] | 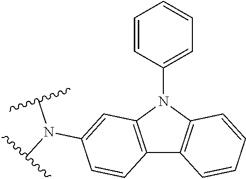 [SMILES: *N(*)c1ccc2c3ccccc3n(-c3ccccc3)c2c1] | 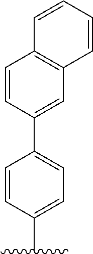 [SMILES: *c1ccc(-c2ccc3ccccc3c2)cc1] |
| Q42 | 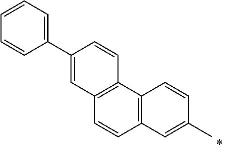 [SMILES: Cc1ccc2c(ccc3cc(-c4ccccc4)ccc32)c1] | 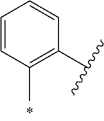 [SMILES: *c1ccccc1C] | 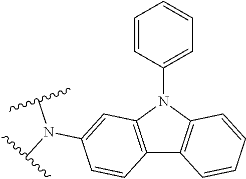 [SMILES: *N(*)c1ccc2c3ccccc3n(-c3ccccc3)c2c1] | 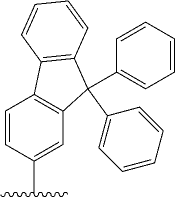 [SMILES: *c1ccc2c(c1)C(c1ccccc1)(c1ccccc1)c1ccccc1-2] |
| Q43 | 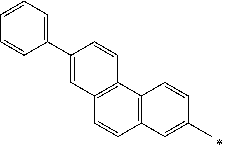 [SMILES: Cc1ccc2c(ccc3cc(-c4ccccc4)ccc32)c1] | 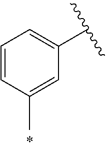 [SMILES: *c1cccc(C)c1] | 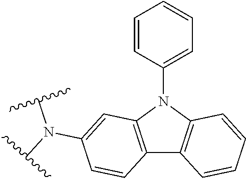 [SMILES: *N(*)c1ccc2c3ccccc3n(-c3ccccc3)c2c1] | 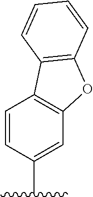 [SMILES: *c1ccc2c(c1)oc1ccccc12] |
| Q44 | 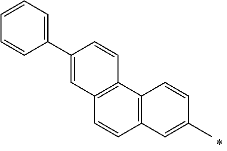 [SMILES: Cc1ccc2c(ccc3cc(-c4ccccc4)ccc32)c1] | 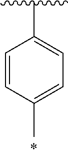 [SMILES: *c1ccc(C)cc1] | 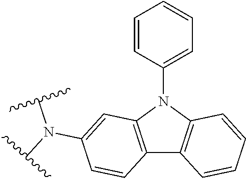 [SMILES: *N(*)c1ccc2c3ccccc3n(-c3ccccc3)c2c1] | 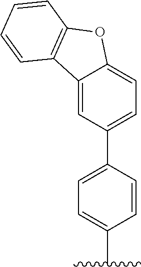 [SMILES: *c1ccc(-c2ccc3oc4ccccc4c3c2)cc1] |
| Q45 | 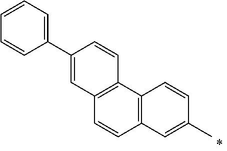 [SMILES: Cc1ccc2c(ccc3cc(-c4ccccc4)ccc32)c1] | 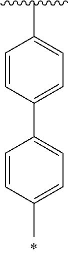 [SMILES: *c1ccc(-c2ccc(C)cc2)cc1] | 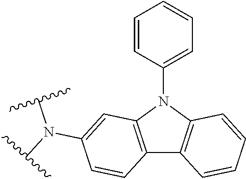 [SMILES: *N(*)c1ccc2c3ccccc3n(-c3ccccc3)c2c1] | 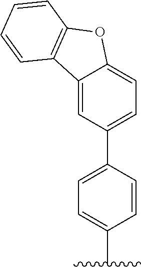 [SMILES: *c1ccc(-c2ccc3oc4ccccc4c3c2)cc1] |
| Q46 | 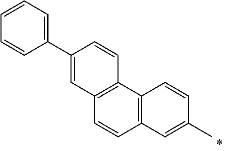 [SMILES: Cc1ccc2c(ccc3cc(-c4ccccc4)ccc32)c1] | 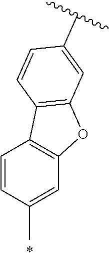 [SMILES: *c1ccc2c(c1)oc1cc(C)ccc12] | 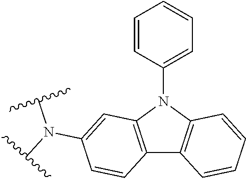 [SMILES: *N(*)c1ccc2c3ccccc3n(-c3ccccc3)c2c1] | 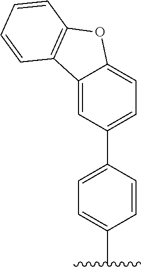 [SMILES: *c1ccc(-c2ccc3oc4ccccc4c3c2)cc1] |
| S1 | 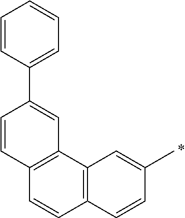 [SMILES: Cc1ccc2ccc3ccc(-c4ccccc4)cc3c2c1] | 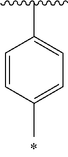 [SMILES: *c1ccc(C)cc1] | 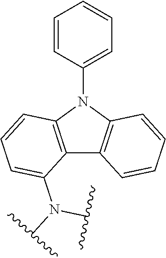 [SMILES: *N(*)c1cccc2c1c1ccccc1n2-c1ccccc1] | 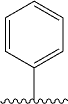 [SMILES: *c1ccccc1] |
| S2 | 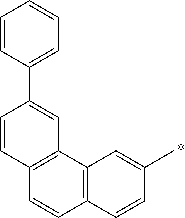 [SMILES: Cc1ccc2ccc3ccc(-c4ccccc4)cc3c2c1] | 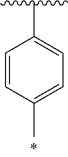 [SMILES: *c1ccc(C)cc1] | 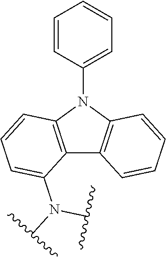 [SMILES: *N(*)c1cccc2c1c1ccccc1n2-c1ccccc1] | 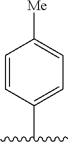 [SMILES: *c1ccc(C)cc1] |
| S3 | 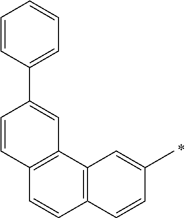 [SMILES: Cc1ccc2ccc3ccc(-c4ccccc4)cc3c2c1] | 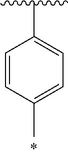 [SMILES: *c1ccc(C)cc1] | 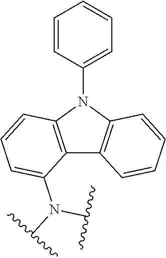 [SMILES: *N(*)c1cccc2c1c1ccccc1n2-c1ccccc1] | 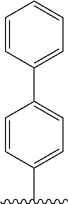 [SMILES: *c1ccc(-c2ccccc2)cc1] |
| S4 | 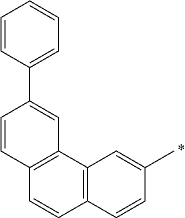 [SMILES: Cc1ccc2ccc3ccc(-c4ccccc4)cc3c2c1] | 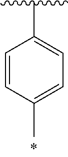 [SMILES: *c1ccc(C)cc1] | 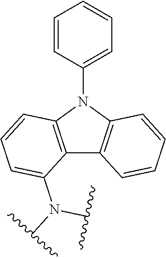 [SMILES: *N(*)c1cccc2c1c1ccccc1n2-c1ccccc1] | 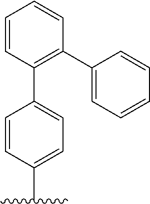 [SMILES: *c1ccc(-c2ccccc2-c2ccccc2)cc1] |
| S5 | 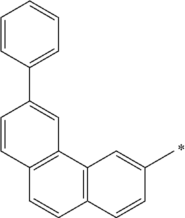 [SMILES: Cc1ccc2ccc3ccc(-c4ccccc4)cc3c2c1] | 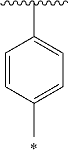 [SMILES: *c1ccc(C)cc1] | 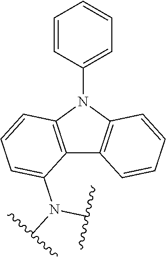 [SMILES: *N(*)c1cccc2c1c1ccccc1n2-c1ccccc1] | 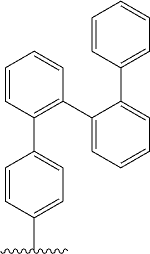 [SMILES: *c1ccc(-c2ccccc2-c2ccccc2-c2ccccc2)cc1] |
| S6 | 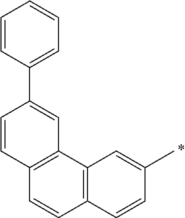 [SMILES: Cc1ccc2ccc3ccc(-c4ccccc4)cc3c2c1] | 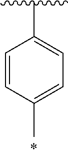 [SMILES: *c1ccc(C)cc1] | 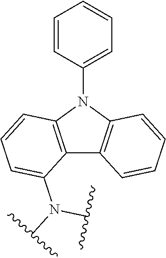 [SMILES: *N(*)c1cccc2c1c1ccccc1n2-c1ccccc1] | 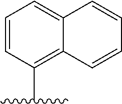 [SMILES: *c1cccc2ccccc12] |
| S7 | 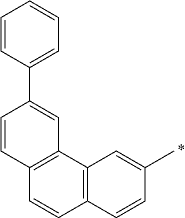 [SMILES: Cc1ccc2ccc3ccc(-c4ccccc4)cc3c2c1] | 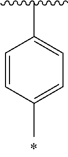 [SMILES: *c1ccc(C)cc1] | 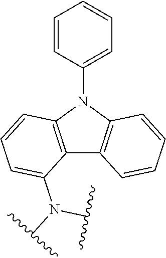 [SMILES: *N(*)c1cccc2c1c1ccccc1n2-c1ccccc1] | 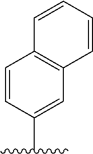 [SMILES: *c1ccc2ccccc2c1] |
| S8 | 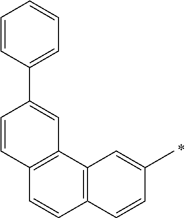 [SMILES: Cc1ccc2ccc3ccc(-c4ccccc4)cc3c2c1] | 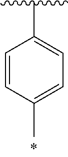 [SMILES: *c1ccc(C)cc1] | 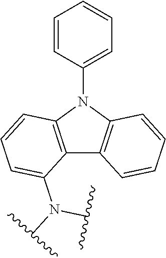 [SMILES: *N(*)c1cccc2c1c1ccccc1n2-c1ccccc1] | 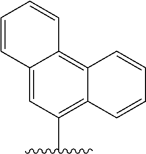 [SMILES: *c1cc2ccccc2c2ccccc12] |
| S9 | 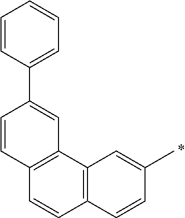 [SMILES: Cc1ccc2ccc3ccc(-c4ccccc4)cc3c2c1] | 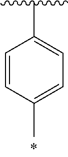 [SMILES: *c1ccc(C)cc1] | 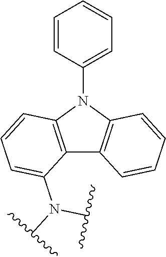 [SMILES: *N(*)c1cccc2c1c1ccccc1n2-c1ccccc1] | 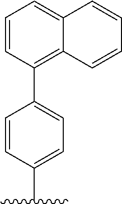 [SMILES: *c1ccc(-c2cccc3ccccc23)cc1] |
| S10 | 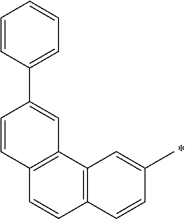 [SMILES: Cc1ccc2ccc3ccc(-c4ccccc4)cc3c2c1] | 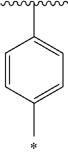 [SMILES: *c1ccc(C)cc1] | 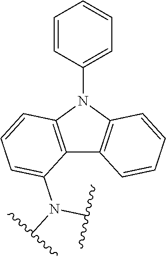 [SMILES: *N(*)c1cccc2c1c1ccccc1n2-c1ccccc1] | 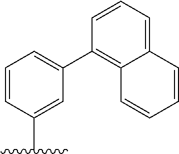 [SMILES: *c1cccc(-c2cccc3ccccc23)c1] |
| S11 | 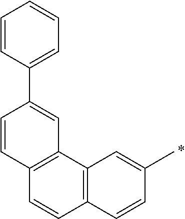 [SMILES: Cc1ccc2ccc3ccc(-c4ccccc4)cc3c2c1] | 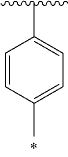 [SMILES: *c1ccc(C)cc1] | 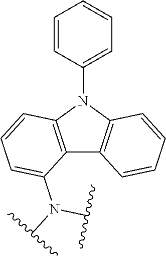 [SMILES: *N(*)c1cccc2c1c1ccccc1n2-c1ccccc1] | 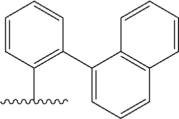 [SMILES: *c1ccccc1-c1cccc2ccccc12] |
| S12 | 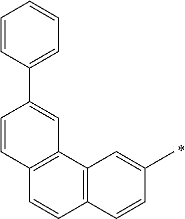 [SMILES: Cc1ccc2ccc3ccc(-c4ccccc4)cc3c2c1] | 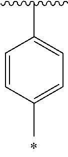 [SMILES: *c1ccc(C)cc1] | 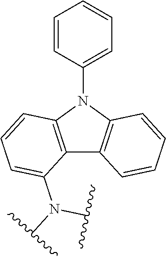 [SMILES: *N(*)c1cccc2c1c1ccccc1n2-c1ccccc1] | 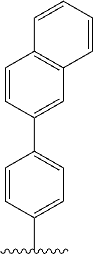 [SMILES: *c1ccc(-c2ccc3ccccc3c2)cc1] |
| S13 | 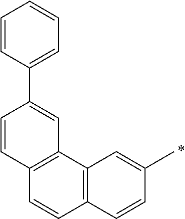 [SMILES: Cc1ccc2ccc3ccc(-c4ccccc4)cc3c2c1] | 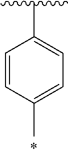 [SMILES: *c1ccc(C)cc1] | 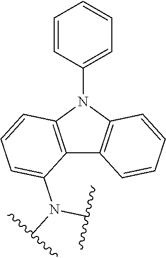 [SMILES: *N(*)c1cccc2c1c1ccccc1n2-c1ccccc1] | 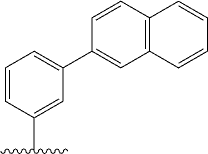 [SMILES: *c1cccc(-c2ccc3ccccc3c2)c1] |
| S14 | 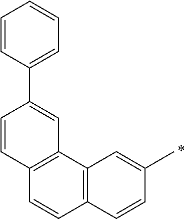 [SMILES: Cc1ccc2ccc3ccc(-c4ccccc4)cc3c2c1] | 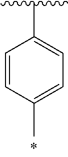 [SMILES: *c1ccc(C)cc1] | 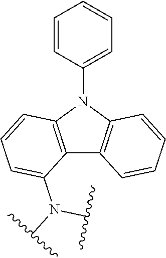 [SMILES: *N(*)c1cccc2c1c1ccccc1n2-c1ccccc1] | 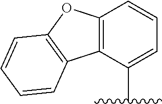 [SMILES: *c1cccc2oc3ccccc3c12] |
| S15 | 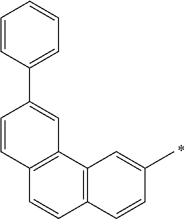 [SMILES: Cc1ccc2ccc3ccc(-c4ccccc4)cc3c2c1] | 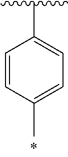 [SMILES: *c1ccc(C)cc1] | 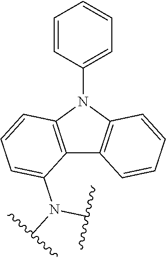 [SMILES: *N(*)c1cccc2c1c1ccccc1n2-c1ccccc1] | 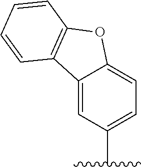 [SMILES: *c1ccc2oc3ccccc3c2c1] |
| S16 | 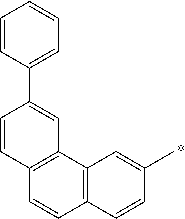 [SMILES: Cc1ccc2ccc3ccc(-c4ccccc4)cc3c2c1] | 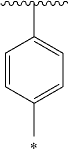 [SMILES: *c1ccc(C)cc1] | 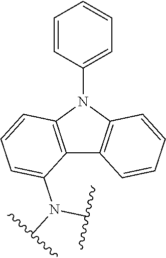 [SMILES: *N(*)c1cccc2c1c1ccccc1n2-c1ccccc1] | 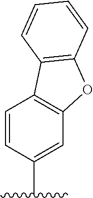 [SMILES: *c1ccc2c(c1)oc1ccccc12] |
| S17 | 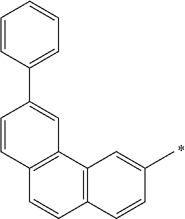 [SMILES: Cc1ccc2ccc3ccc(-c4ccccc4)cc3c2c1] | 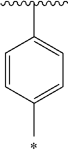 [SMILES: *c1ccc(C)cc1] | 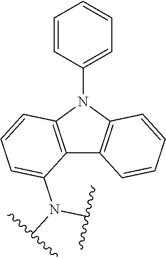 [SMILES: *N(*)c1cccc2c1c1ccccc1n2-c1ccccc1] | 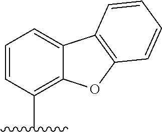 [SMILES: *c1cccc2c1oc1ccccc12] |
| S18 | 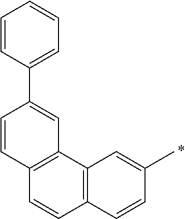 [SMILES: Cc1ccc2ccc3ccc(-c4ccccc4)cc3c2c1] | 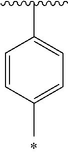 [SMILES: *c1ccc(C)cc1] | 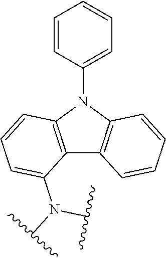 [SMILES: *N(*)c1cccc2c1c1ccccc1n2-c1ccccc1] | 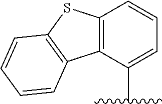 [SMILES: *c1cccc2sc3ccccc3c12] |
| S19 | 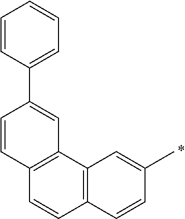 [SMILES: Cc1ccc2ccc3ccc(-c4ccccc4)cc3c2c1] | 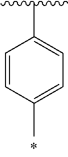 [SMILES: *c1ccc(C)cc1] | 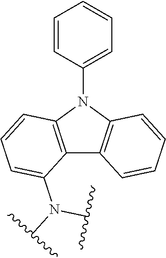 [SMILES: *N(*)c1cccc2c1c1ccccc1n2-c1ccccc1] | 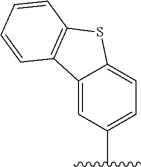 [SMILES: *c1ccc2sc3ccccc3c2c1] |
| S20 | 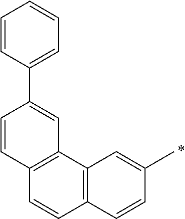 [SMILES: Cc1ccc2ccc3ccc(-c4ccccc4)cc3c2c1] | 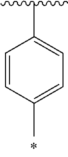 [SMILES: *c1ccc(C)cc1] | 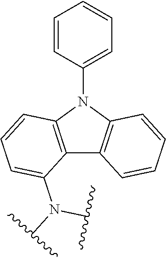 [SMILES: *N(*)c1cccc2c1c1ccccc1n2-c1ccccc1] | 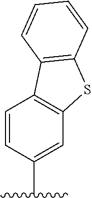 [SMILES: *c1ccc2c(c1)sc1ccccc12] |
| S21 | 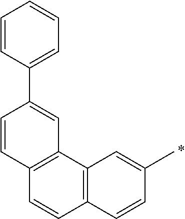 [SMILES: Cc1ccc2ccc3ccc(-c4ccccc4)cc3c2c1] | 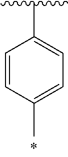 [SMILES: *c1ccc(C)cc1] | 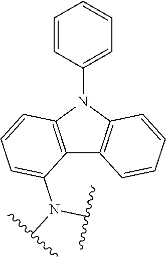 [SMILES: *N(*)c1cccc2c1c1ccccc1n2-c1ccccc1] | 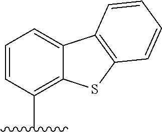 [SMILES: *c1cccc2c1sc1ccccc12] |
| S22 | 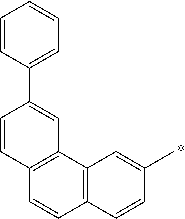 [SMILES: Cc1ccc2ccc3ccc(-c4ccccc4)cc3c2c1] | 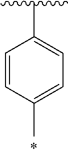 [SMILES: *c1ccc(C)cc1] | 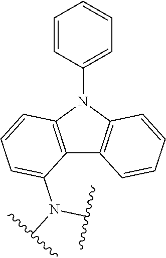 [SMILES: *N(*)c1cccc2c1c1ccccc1n2-c1ccccc1] | 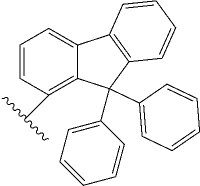 [SMILES: *c1cccc2c1C(c1ccccc1)(c1ccccc1)c1ccccc1-2] |
| S23 | 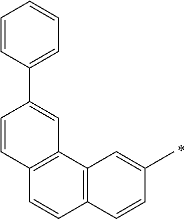 [SMILES: Cc1ccc2ccc3ccc(-c4ccccc4)cc3c2c1] | 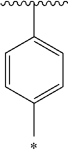 [SMILES: *c1ccc(C)cc1] | 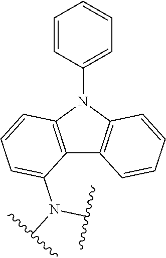 [SMILES: *N(*)c1cccc2c1c1ccccc1n2-c1ccccc1] | 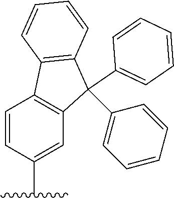 [SMILES: *c1ccc2c(c1)C(c1ccccc1)(c1ccccc1)c1ccccc1-2] |
| S24 | 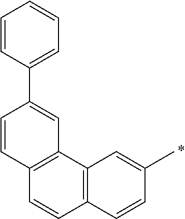 [SMILES: Cc1ccc2ccc3ccc(-c4ccccc4)cc3c2c1] | 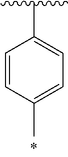 [SMILES: *c1ccc(C)cc1] | 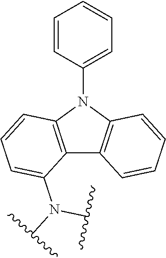 [SMILES: *N(*)c1cccc2c1c1ccccc1n2-c1ccccc1] | 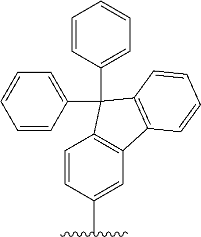 [SMILES: *c1ccc2c(c1)-c1ccccc1C2(c1ccccc1)c1ccccc1] |
| S25 | 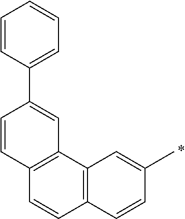 [SMILES: Cc1ccc2ccc3ccc(-c4ccccc4)cc3c2c1] | 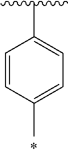 [SMILES: *c1ccc(C)cc1] | 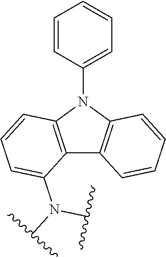 [SMILES: *N(*)c1cccc2c1c1ccccc1n2-c1ccccc1] | 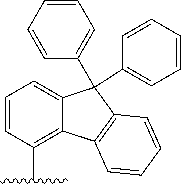 [SMILES: *c1cccc2c1-c1ccccc1C2(c1ccccc1)c1ccccc1] |
| S26 | 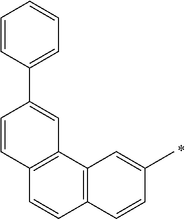 [SMILES: Cc1ccc2ccc3ccc(-c4ccccc4)cc3c2c1] | 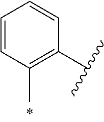 [SMILES: *c1ccccc1C] | 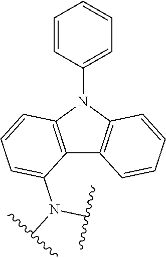 [SMILES: *N(*)c1cccc2c1c1ccccc1n2-c1ccccc1] | 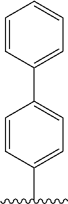 [SMILES: *c1ccc(-c2ccccc2)cc1] |
| S27 | 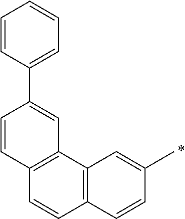 [SMILES: Cc1ccc2ccc3ccc(-c4ccccc4)cc3c2c1] | 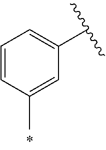 [SMILES: *c1cccc(C)c1] | 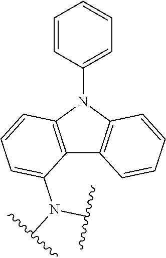 [SMILES: *N(*)c1cccc2c1c1ccccc1n2-c1ccccc1] | 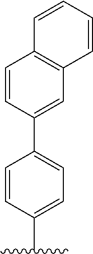 [SMILES: *c1ccc(-c2ccc3ccccc3c2)cc1] |
| S28 | 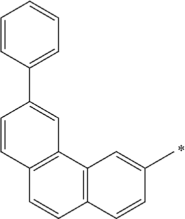 [SMILES: Cc1ccc2ccc3ccc(-c4ccccc4)cc3c2c1] | 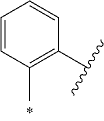 [SMILES: *c1ccccc1C] | 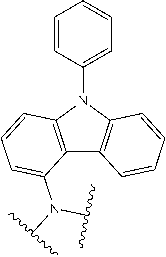 [SMILES: *N(*)c1cccc2c1c1ccccc1n2-c1ccccc1] | 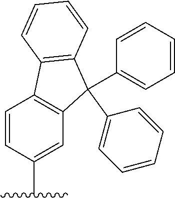 [SMILES: *c1ccc2c(c1)C(c1ccccc1)(c1ccccc1)c1ccccc1-2] |
| S29 | 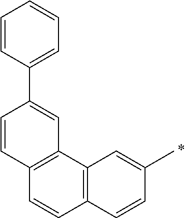 [SMILES: Cc1ccc2ccc3ccc(-c4ccccc4)cc3c2c1] | 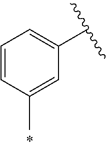 [SMILES: *c1cccc(C)c1] | 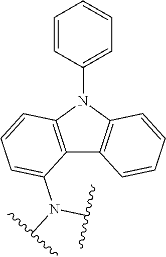 [SMILES: *N(*)c1cccc2c1c1ccccc1n2-c1ccccc1] | 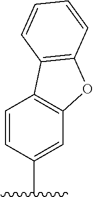 [SMILES: *c1ccc2c(c1)oc1ccccc12] |
| S30 | 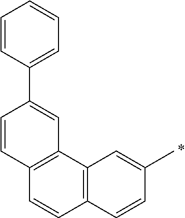 [SMILES: Cc1ccc2ccc3ccc(-c4ccccc4)cc3c2c1] | 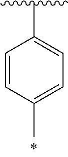 [SMILES: *c1ccc(C)cc1] | 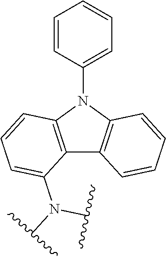 [SMILES: *N(*)c1cccc2c1c1ccccc1n2-c1ccccc1] | 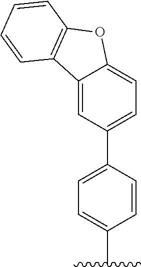 [SMILES: *c1ccc(-c2ccc3oc4ccccc4c3c2)cc1] |
| S31 | 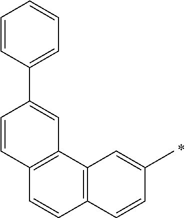 [SMILES: Cc1ccc2ccc3ccc(-c4ccccc4)cc3c2c1] | 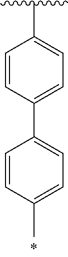 [SMILES: *c1ccc(-c2ccc(C)cc2)cc1] | 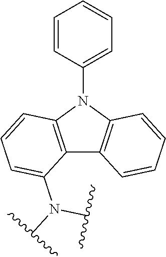 [SMILES: *N(*)c1cccc2c1c1ccccc1n2-c1ccccc1] | 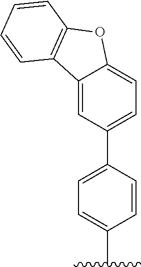 [SMILES: *c1ccc(-c2ccc3oc4ccccc4c3c2)cc1] |
| S32 | 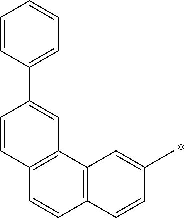 [SMILES: Cc1ccc2ccc3ccc(-c4ccccc4)cc3c2c1] | 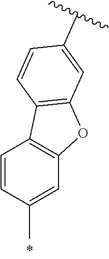 [SMILES: *c1ccc2c(c1)oc1cc(C)ccc12] | 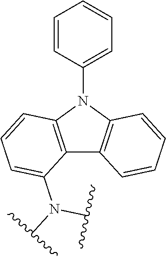 [SMILES: *N(*)c1cccc2c1c1ccccc1n2-c1ccccc1] | 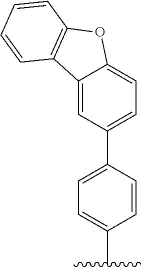 [SMILES: *c1ccc(-c2ccc3oc4ccccc4c3c2)cc1] |
| S33 | 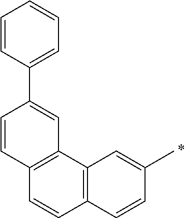 [SMILES: Cc1ccc2ccc3ccc(-c4ccccc4)cc3c2c1] | 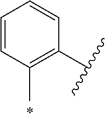 [SMILES: *c1ccccc1C] | 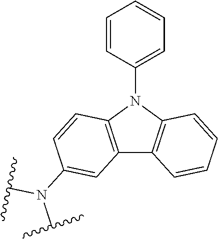 [SMILES: *N(*)c1ccc2c(c1)c1ccccc1n2-c1ccccc1] | 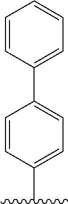 [SMILES: *c1ccc(-c2ccccc2)cc1] |
| S34 | 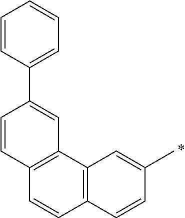 [SMILES: Cc1ccc2ccc3ccc(-c4ccccc4)cc3c2c1] | 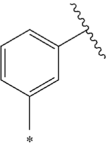 [SMILES: *c1cccc(C)c1] | 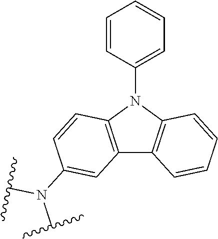 [SMILES: *N(*)c1ccc2c(c1)c1ccccc1n2-c1ccccc1] | 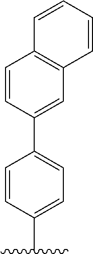 [SMILES: *c1ccc(-c2ccc3ccccc3c2)cc1] |
| S35 | 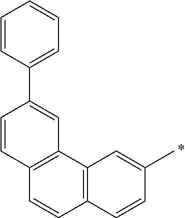 [SMILES: Cc1ccc2ccc3ccc(-c4ccccc4)cc3c2c1] | 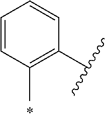 [SMILES: *c1ccccc1C] | 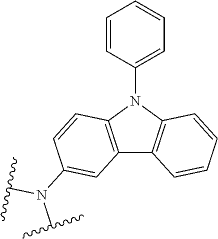 [SMILES: *N(*)c1ccc2c(c1)c1ccccc1n2-c1ccccc1] | 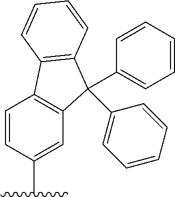 [SMILES: *c1ccc2c(c1)C(c1ccccc1)(c1ccccc1)c1ccccc1-2] |
| S36 | 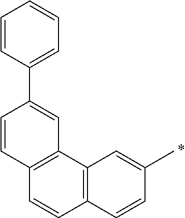 [SMILES: Cc1ccc2ccc3ccc(-c4ccccc4)cc3c2c1] | 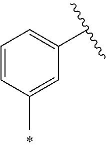 [SMILES: *c1cccc(C)c1] | 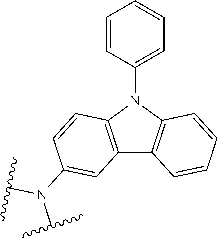 [SMILES: *N(*)c1ccc2c(c1)c1ccccc1n2-c1ccccc1] | 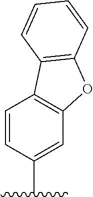 [SMILES: *c1ccc2c(c1)oc1ccccc12] |
| S37 | 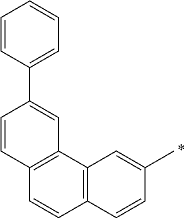 [SMILES: Cc1ccc2ccc3ccc(-c4ccccc4)cc3c2c1] | 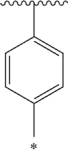 [SMILES: *c1ccc(C)cc1] | 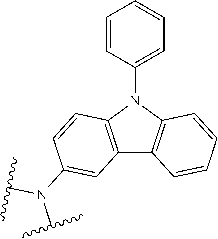 [SMILES: *N(*)c1ccc2c(c1)c1ccccc1n2-c1ccccc1] | 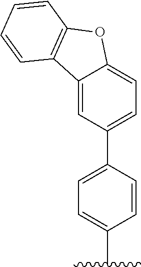 [SMILES: *c1ccc(-c2ccc3oc4ccccc4c3c2)cc1] |
| S38 | 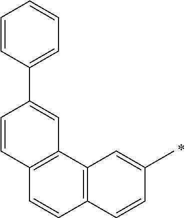 [SMILES: Cc1ccc2ccc3ccc(-c4ccccc4)cc3c2c1] | 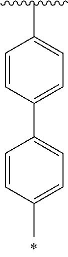 [SMILES: *c1ccc(-c2ccc(C)cc2)cc1] | 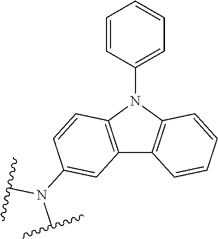 [SMILES: *N(*)c1ccc2c(c1)c1ccccc1n2-c1ccccc1] | 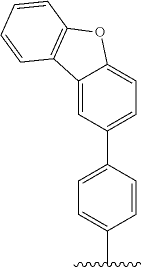 [SMILES: *c1ccc(-c2ccc3oc4ccccc4c3c2)cc1] |
| S39 | 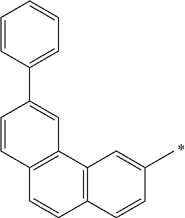 [SMILES: Cc1ccc2ccc3ccc(-c4ccccc4)cc3c2c1] | 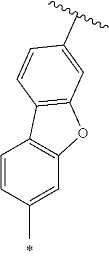 [SMILES: *c1ccc2c(c1)oc1cc(C)ccc12] | 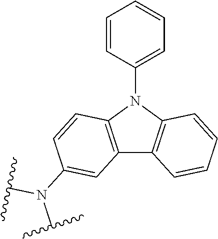 [SMILES: *N(*)c1ccc2c(c1)c1ccccc1n2-c1ccccc1] | 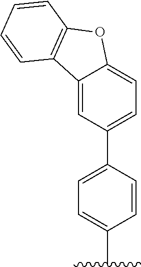 [SMILES: *c1ccc(-c2ccc3oc4ccccc4c3c2)cc1] |
| S40 | 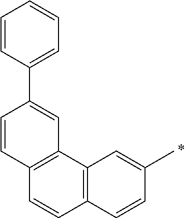 [SMILES: Cc1ccc2ccc3ccc(-c4ccccc4)cc3c2c1] | 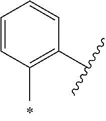 [SMILES: *c1ccccc1C] | 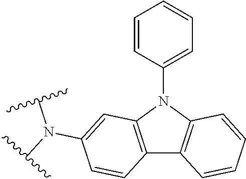 [SMILES: *N(*)c1ccc2c3ccccc3n(-c3ccccc3)c2c1] | 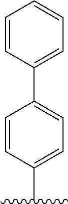 [SMILES: *c1ccc(-c2ccccc2)cc1] |
| S41 | 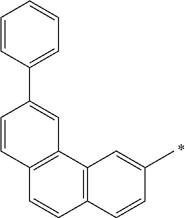 [SMILES: Cc1ccc2ccc3ccc(-c4ccccc4)cc3c2c1] | 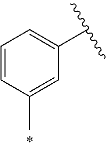 [SMILES: *c1cccc(C)c1] | 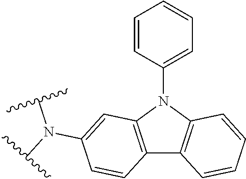 [SMILES: *N(*)c1ccc2c3ccccc3n(-c3ccccc3)c2c1] | 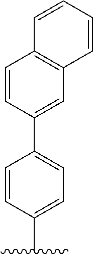 [SMILES: *c1ccc(-c2ccc3ccccc3c2)cc1] |
| S42 | 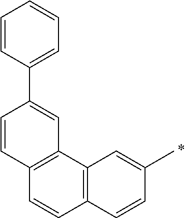 [SMILES: Cc1ccc2ccc3ccc(-c4ccccc4)cc3c2c1] | 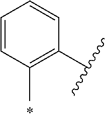 [SMILES: *c1ccccc1C] | 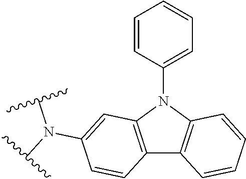 [SMILES: *N(*)c1ccc2c3ccccc3n(-c3ccccc3)c2c1] | 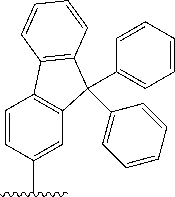 [SMILES: *c1ccc2c(c1)C(c1ccccc1)(c1ccccc1)c1ccccc1-2] |
| S43 | 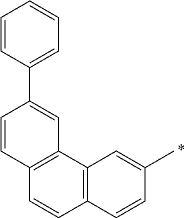 [SMILES: Cc1ccc2ccc3ccc(-c4ccccc4)cc3c2c1] | 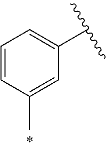 [SMILES: *c1cccc(C)c1] | 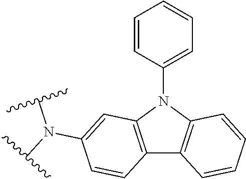 [SMILES: *N(*)c1ccc2c3ccccc3n(-c3ccccc3)c2c1] | 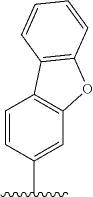 [SMILES: *c1ccc2c(c1)oc1ccccc12] |
| S44 | 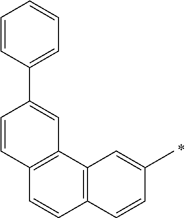 [SMILES: Cc1ccc2ccc3ccc(-c4ccccc4)cc3c2c1] | 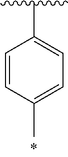 [SMILES: *c1ccc(C)cc1] | 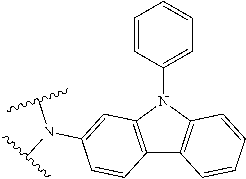 [SMILES: *N(*)c1ccc2c3ccccc3n(-c3ccccc3)c2c1] | 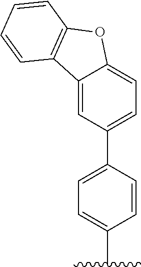 [SMILES: *c1ccc(-c2ccc3oc4ccccc4c3c2)cc1] |
| S45 | 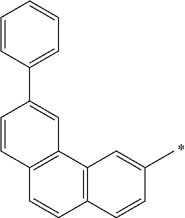 [SMILES: Cc1ccc2ccc3ccc(-c4ccccc4)cc3c2c1] | 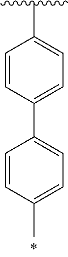 [SMILES: *c1ccc(-c2ccc(C)cc2)cc1] | 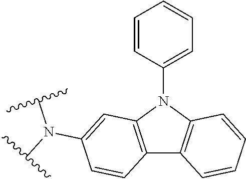 [SMILES: *N(*)c1ccc2c3ccccc3n(-c3ccccc3)c2c1] | 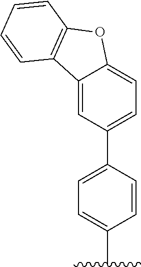 [SMILES: *c1ccc(-c2ccc3oc4ccccc4c3c2)cc1] |
| S46 | 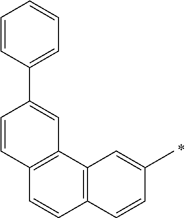 [SMILES: Cc1ccc2ccc3ccc(-c4ccccc4)cc3c2c1] | 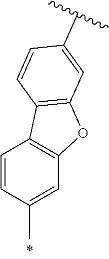 [SMILES: *c1ccc2c(c1)oc1cc(C)ccc12] | 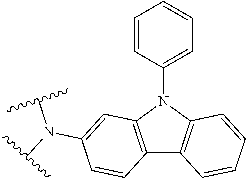 [SMILES: *N(*)c1ccc2c3ccccc3n(-c3ccccc3)c2c1] | 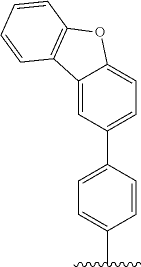 [SMILES: *c1ccc(-c2ccc3oc4ccccc4c3c2)cc1] |

13. An amine compound represented by Formula 1: 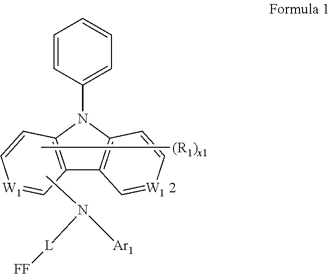
wherein, in Formula 1,
Ar₁ is a substituted or unsubstituted aryl group having 6 to 50 ring-forming carbon atoms, or a substituted or unsubstituted heteroaryl group having 2 to 50 ring-forming carbon atoms, and Ar₁ is not a substituted or unsubstituted carbazole group,
L is a substituted or unsubstituted arylene group having 6 to 50 ring-forming carbon atoms, or a substituted or unsubstituted heteroarylene group having 2 to 50 ring-forming carbon atoms,
R₁ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring,
x1 is an integer from 0 to 5,
W₁ and W₂ are each independently CRₐ or a carbon atom bonded to the nitrogen atom in Formula 1,
Rₐ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring,
FF is represented by Formula 2, and
in Formula 2, —* is a position linked to L, and 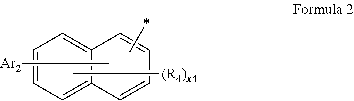
wherein, in Formula 2,
Ar₂ is a substituted or unsubstituted aryl group having 6 to 50 ring-forming carbon atoms, and Ar₂ is not an aryl group substituted with an amine group,
R₄ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, and
x4 is an integer from 0 to 6,
wherein, when R₄ does not form a ring with an adjacent group, each of W₁ and W₂ in Formula 1 is CRₐ, and
wherein, when Formula 2 is represented by 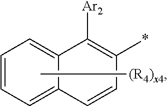
R₄ does not form a ring with an adjacent group, and Ar₁ is a substituted or unsubstituted fluorene group, and the nitrogen atom in Formula 1 is not linked to the second position of the substituted or unsubstituted fluorene group, Ar₁.

14. The amine compound of claim 13, wherein FF is represented by Formula 2a: 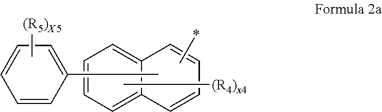
wherein, in Formula 2a,
R₅ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring,
x5 is an integer from 0 to 5, and
R₄ and x4 are the same as defined in Formula 2.

15. The amine compound of claim 13, wherein FF is represented by any one selected from among Formula 2-1 to Formula 2-5: 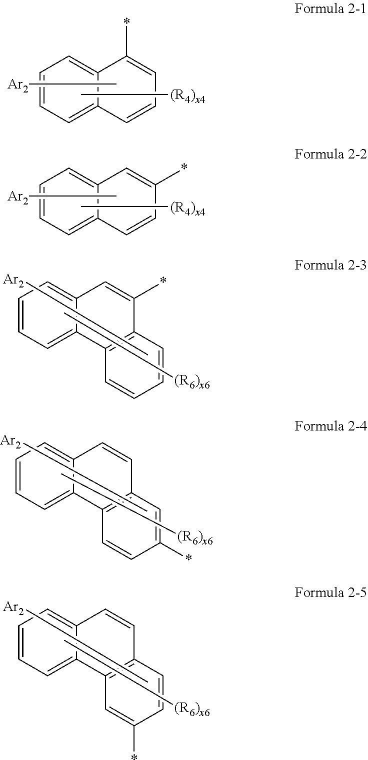
wherein, in Formula 2-3 to Formula 2-5,
R₆ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, and
x6 is an integer from 0 to 8, and
in Formula 2-1 to Formula 2-5,
Ar₂, R₄, and x4 are the same as defined in Formula 2.

16. The amine compound of claim 13, wherein the amine compound represented by Formula 1 is represented by any one selected from among Formula 3-1 to Formula 3-3: 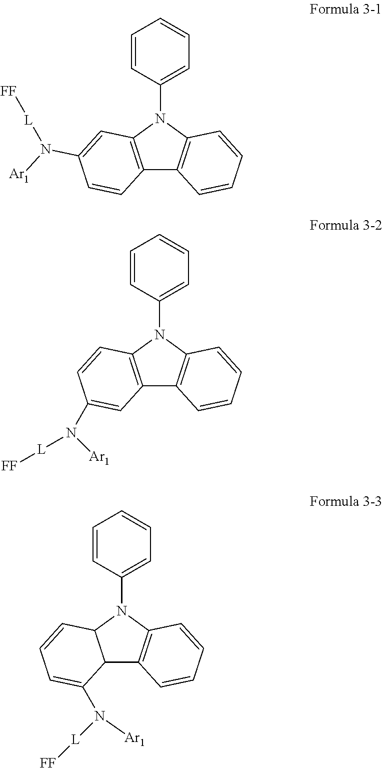
wherein, in Formula 3-1 to Formula 3-3, L, Ar₁, and FF are the same as defined in Formula 1.

17. The amine compound of claim 16, wherein L is a substituted or unsubstituted phenylene group, a substituted or unsubstituted biphenylene group, or a substituted or unsubstituted divalent dibenzofuran group.

18. A display device comprising a plurality of light emitting elements, wherein each of the light emitting elements comprises:
a first electrode;
a second electrode on the first electrode; and
an emission layer between the first electrode and the second electrode; and
a hole transport region which is between the first electrode and the emission layer and comprises an amine compound represented by Formula 1: 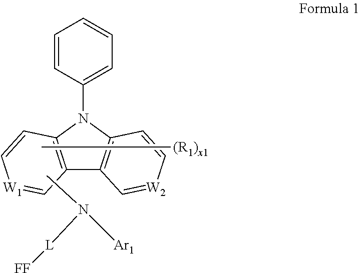
wherein, in Formula 1,
Ar₁ is a substituted or unsubstituted aryl group having 6 to 50 ring-forming carbon atoms, or a substituted or unsubstituted heteroaryl group having 2 to 50 ring-forming carbon atoms, and Ar₁ is not a substituted or unsubstituted carbazole group,
L is a substituted or unsubstituted arylene group having 6 to 50 ring-forming carbon atoms, or a substituted or unsubstituted heteroarylene group having 2 to 50 ring-forming carbon atoms,
R₁ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring,
x1 is an integer from 0 to 5,
W₁ and W₂ are each independently CRₐ or a carbon atom bonded to the nitrogen atom in Formula 1,
Rₐ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring,
FF is represented by Formula 2, and
in Formula 2, —* is a position linked to L, and 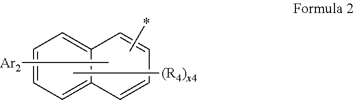
wherein, in Formula 2,
Ar₂ is a substituted or unsubstituted aryl group having 6 to 50 ring-forming carbon atoms, and Ar₂ is not an aryl group substituted with an amine group,
R₄ is a hydrogen atom, a deuterium atom, a halogen atom, a cyano group, a substituted or unsubstituted silyl group, a substituted or unsubstituted thio group, a substituted or unsubstituted oxy group, a substituted or unsubstituted alkyl group having 1 to 20 carbon atoms, a substituted or unsubstituted aryl group having 6 to 50 ring-forming carbon atoms, or a substituted or unsubstituted heteroaryl group having 2 to 50 ring-forming carbon atoms, or is bonded to an adjacent group to form a ring, and
x4 is an integer from 0 to 6,
wherein, when R₄ does not form a ring with an adjacent group, each of W₁ and W₂ in Formula 1 is CRₐ, and
wherein, when Formula 2 is represented by 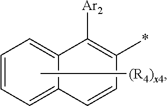
R₄ does not form a ring with an adjacent group, and Ar₁ is a substituted or unsubstituted fluorene group, and the nitrogen atom in Formula 1 is not linked to the second position of the substituted or unsubstituted fluorene group, Ar₁.

19. The display device of claim 18, wherein the plurality of light emitting elements comprise:
a first light emitting element comprising a first emission layer configured to emit light having a first wavelength;
a second light emitting element comprising a second emission layer configured to emit light having a second wavelength different from the first wavelength and is spaced apart from the first emission layer on a plane; and
a third light emitting element configured to emit light having a third wavelength different from the first wavelength and the second wavelength and is spaced apart from the first emission layer and the second emission layer on a plane.

20. The display device of claim 19, wherein the first wavelength is longer than the second wavelength, and the second wavelength is longer than the third wavelength.
